(19) United States
(12) Patent Application Publication
(10) Pub. No.: US 2021/0214440 A1
(43) Pub. Date: Jul. 15, 2021

(54) MATERIALS AND METHODS FOR IN VIVO BIOLOGICAL TARGETING

(71) Applicant: Janssen Biotech, Inc., Horsham, PA (US)

(72) Inventors: Rajkumar Ganesan, Blue Bell, PA (US); Sanjaya Singh, Blue Bell, PA (US); Iqbal S. Grewal, Newtown, PA (US); Michael Riis Hansen, Broomall, PA (US)

(73) Assignee: Janssen Biotech, Inc., Horsham, PA (US)

(21) Appl. No.: 17/125,162

(22) Filed: Dec. 17, 2020

Related U.S. Application Data
(60) Provisional application No. 63/091,100, filed on Oct. 13, 2020.
(60) Provisional application No. 62/949,526, filed on Dec. 18, 2019.
(60) Provisional application No. 62/949,519, filed on Dec. 18, 2019.
(60) Provisional application No. 62/949,513, filed on Dec. 18, 2019.
(60) Provisional application No. 62/949,507, filed on Dec. 18, 2019.
(60) Provisional application No. 62/949,502, filed on Dec. 18, 2019.
(60) Provisional application No. 62/949,499, filed on Dec. 18, 2019.
(60) Provisional application No. 62/949,492, filed on Dec. 18, 2019.
(60) Provisional application No. 62/949,486, filed on Dec. 18, 2019.

(51) Int. Cl. C07K 16/28; B01D 15/00

(52) U.S. Cl. CPC C07K 16/2815; A61K 2039/507; B01D 15/00; C07K 16/2809

(57) ABSTRACT

An isolated molecule, comprising: a first antigen binding domain and a second antigen binding domain, wherein the first antigen binding domain specifically binds CD8 and the second antigen binding domain specifically binds a T cell receptor (TCR) complex.

### CROSS-REFERENCE TO RELATED APPLICATIONS

This application claims benefit of priority of U.S. Ser. No. 62/949,486 filed on Dec. 18, 2019, U.S. Ser. No. 62/949,492 filed on Dec. 18, 2019, U.S. Ser. No. 62/949,499 filed on Dec. 18, 2019, U.S. Ser. No. 62/949,502 filed on Dec. 18, 2019, U.S. Ser. No. 62/949,507 filed on Dec. 18, 2019, U.S. Ser. No. 62/949,513 filed on Dec. 18, 2019, U.S. Ser. No. 62/949,519 filed on Dec. 18, 2019, U.S. Ser. No. 62/949,526 filed on Dec. 18, 2019, and U.S. Ser. No. 63/091,100 filed on Oct. 13, 2020, the contents of each of which is incorporated herein by reference in its entirety.

### SEQUENCE LISTING

This application incorporates by reference a Sequence Listing submitted with this application as a text format, entitled “14620-329-999_SL.txt,” created on Dec. 15, 2020 and having a size of 1,037,532 bytes.

### TECHNICAL FIELD

Provided herein are molecules comprising multiple binding domains, compositions comprising same, and methods for uses thereof, e.g., for treating a disease or disorder such as cancer.

### BACKGROUND

T cell redirection has become an alternative to cancer therapies with the approval of BENLYSTA® (blinatumomab). T cell redirection utilizing CD3 binding domains however poses challenges as the approach results in unselective recruitment of pan-T cells, including exhausted T cells, helper and regulatory cells such as CD4⁺, Th1, Th2, Th9, Th17, Th22, Tfh, Tregs, Tr1 and non-CTL CD8⁺ cells, i.e., cells that are incapable of mediating tumor cell lysis. Only fraction of the cells recruited by engaging CD3 are cytotoxic T lymphocytes (CTLs). Further, even low doses of T cell redirection molecules based on CD3 may result in cytokine release syndrome. Therefore, there is a need to develop additional strategies to redirect subsets of T cells to enhance selectivity and safety profile of T cell redirecting molecules for improved treatment of cancers and other diseases in which depletion or partial depletion of cells contributing to disease pathogenesis is beneficial.

### SUMMARY

In one aspect, the disclosure provides an isolated molecule, comprising: a first antigen binding domain and a second antigen binding domain, wherein the first antigen binding domain specifically binds CD8 and the second antigen binding domain specifically binds a T cell receptor (TCR) complex.

In another aspect, the disclosure provides an isolated molecule, comprising: a first antigen binding domain, a second antigen binding domain and a third antigen binding domain, wherein the first antigen binding domain specifically binds CD8, the second antigen binding domain specifically binds a TCR complex, and the third antigen binding domain binds a third antigen.

In another aspect, the disclosure provides an isolated molecule, comprising: a first antigen binding domain, a second antigen binding domain and a third antigen binding domain, wherein the first antigen binding domain specifically binds CD8, the second antigen binding domain specifically binds a TCR complex, and the third antigen binding domain binds an antigen expressed by an undesired cell.

In some embodiments, the molecule further comprises a third antigen binding domain that specifically binds an third antigen. In some embodiments, the third antigen comprises an antigen expressed by undesired cells.

In some embodiments, the isolated molecule activates or recruits CD8+ CTLs upon co-engagement of the TCR complex and CD8. In some embodiments, the isolated molecule is unable to activate or recruit CD8+ CTLs in the absence of co-engagement of the TCR complex and CD8. In some embodiments, the first antigen binding domain specifically binds CD8 and the second antigen binding domain specifically binds the TCR complex with affinities that result in activation or recruitment of CD8+ CTLs only upon co-engagement of the TCR complex and CD8.

In some embodiments, the first antigen binding domain, the second antigen binding domain or the third antigen binding domain comprises a scFv, a Fab, a Fab′, a F(ab′)2, a Fd, a Fv, a domain antibody (dAb), a VHH, a heavy chain variable domain (VH), a light chain variable domain (VL), a non-antibody scaffold, or fragments thereof. In some embodiments, the first antigen binding domain comprises the Fab. In some embodiments, the second antigen binding domain comprises the scFv. In some embodiments, the third antigen binding domain comprises the scFv.

In some embodiments, the first antigen binding domain comprising the Fab, the second antigen binding domain comprising the scFv or the third antigen binding domain comprising the scFv is conjugated to the Fc or the fragment of the Fc, to the VH that is capable of specifically biding CD8, to the CL domain or to the CH3 domain via a linker. In some embodiments, the linker comprises a polypeptide of SEQ ID NOs: 2183-2290. In some embodiments, the fragment of the Fc comprises a CH2 domain and a CH3 domain. In some embodiments, the CH3 domain comprises one or more substitutions when compared to a wild-type CH3 domain. In some embodiments, the one or more substitutions comprise T350V, L351Y, F405A, Y407V, T366Y, T366W, F405W, T394W, T394S, Y407T, Y407A, T3665/L368A/Y407V, L351Y/F405A/Y407V, T366I/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V or T350V/T366L/K392L/T394W, wherein residue numbering is according to the EU index.

In yet another aspect, the disclosure also provides an isolated molecule, comprising: a first polypeptide, a second polypeptide and a third polypeptide, wherein the first polypeptide comprises, from N- to C-terminus, a second antigen binding domain comprising a scFv that specifically binds a TCR complex, a VH that is capable of specifically binding CD8, a CH1 domain, a hinge, a CH2 domain and a CH3 domain; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc.

In yet another aspect, the disclosure also provides an isolated molecule, comprising: a first polypeptide, a second polypeptide and a third polypeptide, wherein the first polypeptide comprises, from N- to C-terminus, a VH that is capable of specifically binding CD8, a CH1 domain, a hinge, a CH2 domain and a CH3 domain; the second polypeptide comprises, from N- to C-terminus, a VL that is capable of specifically binding CD8, a CL domain and a second antigen binding domain comprising a scFv that specifically binds a TCR complex; and the third polypeptide comprises, from N- to C-terminus, a third antigen binding domain comprising a scFv that specifically binds an antigen expressed by an undesired cell and a Fc or a fragment of the Fc.

In yet another aspect, the disclosure also provides an isolated molecule, comprising: a first polypeptide, a second polypeptide and a third polypeptide, wherein the first polypeptide comprises, from N- to C-terminus, a VH that is capable of specifically CD8, a CH1 domain, a hinge, a CH2 domain, a CH3 domain and a second antigen binding domain comprising a scFv that specifically binds a TCR complex; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc.

In some embodiments, the first polypeptide comprises a CH3 domain comprising one or more substitutions when compared to a wild-type CH3 domain which promote heterodimerization of the first polypeptide with the third polypeptide; the third polypeptide comprises a CH3 domain comprising one or more substitutions when compared to the wild-type CH3 domain which promote heterodimerization of the third polypeptide with the first polypeptide; or the first polypeptide comprises the CH3 domain comprising one or more substitutions when compared to the wild-type CH3 which promote heterodimerization of the first polypeptide with the third polypeptide and the third polypeptide comprises the CH3 domain comprising one or more substitutions when compared to the wild-type CH3 which promote heterodimerization of the third polypeptide with the first polypeptide.

In some embodiments, the one or more substitutions comprise T350V, L351Y, F405A, Y407V, T366Y, T366W, F405W, T394W, T394S, Y407T, Y407A, T366S/L368A/Y407V, L351Y/F405A/Y407V, T366I/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V or T350V/T366L/K392L/T394W, wherein residue numbering is according to the EU index.

In some embodiments, the Fc, the CH2 domain or the CH3 domain is an IgG1, IgG2, IgG3 or IgG4 isotype. In some embodiments, the second antigen binding domain specifically binds CD3, TCRα chain, TCRβ chain, TCRγ chain or TCRδ chain, or any combination thereof. In some embodiments, the TCRβ chain comprises TCRVB17. In some embodiments, CD3 comprises CD3ε, CD3γ, CD3δ or CD3ζ. In some embodiments, the second antigen binding domain that specifically binds CD3 comprises a heavy chain complementarity determining region 1 (HCDR1 of SEQ ID NO: 2291, a HCDR2 of SEQ ID NO: 2292, a HCDR3 of SEQ ID NO: 2293, a LCDR1 of SEQ ID NO: 2294, a LCDR2 of SEQ ID NO: 2295 and a LCDR3 of SEQ ID NO: 2296. In some embodiments, the second antigen binding domain that specifically binds CD3 comprises the VH of SEQ ID NO: 2297 and the VL of SEQ ID NO: 2298. In some embodiments, the first antigen binding domain comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312. In some embodiments, the first antigen binding domain comprises the VH of SEQ ID NO: 2313 and the VL of SEQ ID NO: 2314.

In some embodiments, the undesired cell is a pathogenic cell. In some embodiments, the undesired cell is a cancer cell, an infected cell, a virus infected cell, a bacterial infected cell, an immune cell, an inflamed cell, a damaged cells, a foreign cell, an apoptotic cell, a dysplastic cell, an immunogenic cell, a metaplastic cell or a mutant cell, or any combination thereof. In some embodiments, the isolated molecule is an antibody or a non-antibody molecule. In some embodiments, the antibody comprises a first half molecule and a second half molecule, wherein the first half molecule comprises the first antigen binding domain and the second antigen binding domain and the second half molecule comprises the third antigen binding domain.

In some embodiments, the antigen expressed by the undesired cell comprises mesothelin, alpha-fetoprotein (ALP), BAGE, BCR-ABL, beta-catenin, beta-HCG, BrE3-antigen, BCA225, BCMA, BTAA, CA125, CA195, CA242, CA-50, CAM43, CAMEL, CAP-1, carbonic anhydrase IX, CA19-9, CA72-4, CAM 17.1, CASP-8, CCCL19, CCCL21, CD1, CD 1a, CD2, CD4, CD5, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD47, CD52, CD54, CD55, CD59, CD64, CD66a-e, CD67, CD68, CD70, CD70L, CD74, CD79a, CD79b, CD80, CD83, CD95, CD123, CD126, CD132, CD133, CD138, CD147, CD154, CDC27, CDK4, CDK4m, CDKN2A, CO-029, CTLA4, CXCR4, CXCR7, CXCL12, HIF-1a, colon-specific antigen-p (CSAp), CEACAM5) CEACAM6, c-Met, DAM, E2A-PRL, EGFR, EGFRvIII, EGP-1, EGP-2, ELF2-M, Ep-CAM, FGF, FGF-5, Flt-1, Flt-3, folate receptor, G250 antigen, Ga733VEpCAM, GAGE, gplOO, GRO-b, H4-RET, HLA-DR, HM1.24, human chorionic gonadotropin (HCG) HER2, HER3, HMGB-1, HIF-1, HSP70-2M, HST-2, HTgp-175, 1a, IGF-1R, IFN-g, IFN-α, IFN-b, IFN-1, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KLK2, KSA, KS-1-antigen, KS1-4, LAGE-1a, Le-Y, LDR/FUT, M344, MA-50, macrophage migration inhibitory factor (MIF), MAGE, MAGE-1, MAGE-3, MAGE-4, MAGE-5, MAGE-6, MART-1, MART-2, TRAG-3, MCP-1, MIP-1A, MIP-1B, MIF, MG7-Ag, MOV18, MUC1, MUC2, MUC3, MUC4, MUC5ac, MUC13, MUC16, MUM-1/2, MUM-3, MYL-RAR, NB/70K, Nm23H1, NuMA, NCA66, NCA95, NCA90, NY-ESO-1, p15, p16, p185erbB2, p180erbB3, PAM4 antigen, pancreatic cancer mucin, PD-1, PD-L1, PD-L2, PI5, placental growth factor, p53, PLAGL2, Pmel17 prostatic acid phosphatase, PSA, PRAME, PSMA, PlGF, ILGF, ILGF-1R, IL-6, IL-25, RCAS1, RS5, RAGE, RANTES, Ras, T101, SAGE, S100, SLAMF7, survivin, survivin-2B, SDDCAG16, TA-90\Mac2 binding protein, TAAL6, TAC, TAG-72, TLP, tenascin, TMEFF2, TRAIL receptors, TRP-1, TRP-2, TSP-180, VEGFR, ED-B fibronectin, WT-1, 17-1A-antigen, C3, C3a, C3b, C5a, C5, bcl-2, K-ras, tumor neoantigen, a viral antigen associated with cancer, FcγRIIB, IL-12β2R, CD28, CD56, CD11c, CD66b, CD41, CD61, CD62, CD235a, CD146, CD326, or CD203c.

In yet another aspect, provided herein is a kit, comprising the isolated molecule provided herein. In some embodiments, the kit further comprises means for diluting or administering the isolated molecule provided herein. In yet another aspect, provided herein is a pharmaceutical composition, comprising the isolated molecule provided herein and a pharmaceutically acceptable excipient.

In yet another aspect, the disclosure provides a method of selectively activating or recruiting CD8+ CTLs towards an undesired cell, comprising: contacting a population of lymphocytes with an isolated molecule comprising: a first antigen binding domain, a second antigen binding domain and a third antigen binding domain, wherein the first antigen binding domain specifically binds CD8, the second antigen binding domain specifically binds a TCR complex and the third antigen binding domain specifically binds an antigen expressed by an undesired cell, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure also provides a method of selectively activating or recruiting CD8⁺ CTLs towards an undesired cell, comprising: contacting a population of lymphocytes with an isolated molecule comprising a first polypeptide, a second polypeptide and a third polypeptide, wherein the first polypeptide comprises, from N- to C-terminus, a second antigen binding domain comprising a scFv that specifically binds a TCR complex, a VH that is capable of specifically binding CD8, a CH1 domain, a hinge, a CH2 domain and a CH3 domain; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure also provides a method of selectively activating or recruiting CD8⁺ CTLs towards an undesired cell, comprising: contacting a population of lymphocytes with an isolated molecule comprising a first polypeptide, a second polypeptide and a third polypeptide, wherein the first polypeptide comprises, from N- to C-terminus, a VH that is capable of specifically binding CD8, a CH1 domain, a hinge, a CH2 domain and a CH3 domain; the second polypeptide comprises, from N- to C-terminus, a VL that is capable of specifically binding CD8, a CL domain and a second antigen binding domain comprising a scFv that specifically binds a TCR complex; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure also provides a method of selectively activating or recruiting CD8⁺ CTLs towards an undesired cell, comprising: contacting a population of lymphocytes with an isolated molecule comprising a first polypeptide, a second polypeptide and a third polypeptide, wherein the first polypeptide comprises, from N- to C-terminus, a VH that is capable of specifically CD8, a CH1 domain, a hinge, a CH2 domain, a CH3 domain and a second antigen binding domain comprising a scFv that specifically binds a TCR complex; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure also provides a method of selectively activating or recruiting CD8⁺ CTLs towards an undesired cell in a subject, comprising: administering to the subject an isolated molecule comprising a first antigen binding domain, a second antigen binding domain and a third antigen binding domain, wherein the first antigen binding domain specifically binds CD8, the second antigen binding domain specifically binds a TCR complex and the third antigen binding domain specifically binds an antigen expressed by an undesired cell, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure provides a method of providing an improved T cell redirection therapy for a subject in need thereof, comprising: administering to the subject an isolated molecule comprising a first antigen binding domain, a second antigen binding domain and a third antigen binding domain, wherein the first antigen binding domain specifically binds CD8, the second antigen binding domain specifically binds a TCR complex and the third antigen binding domain specifically binds an antigen expressed by an undesired cell, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure also provides a method of providing an improved T cell redirection therapy to a subject in need thereof, comprising: administering to the subject an isolated molecule comprising a first polypeptide, a second polypeptide and a third polypeptide, wherein the first polypeptide comprises, from N- to C-terminus, a second antigen binding domain comprising a scFv that specifically binds a TCR complex, a VH that is capable of specifically binding CD8, a CH1 domain, a hinge, a CH2 domain and a CH3 domain; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure also provides a method of providing an improved T cell redirection therapy to a subject in need thereof, comprising: administering to the subject an isolated molecule comprising a first polypeptide, a second polypeptide and a third polypeptide, wherein the first polypeptide comprises, from N- to C-terminus, a VH that is capable of specifically binding CD8, a CH1 domain, a hinge, a CH2 domain and a CH3 domain; the second polypeptide comprises, from N- to C-terminus, a VL that is capable of specifically binding CD8, a CL domain and a second antigen binding domain comprising a scFv that specifically binds a TCR complex; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure also provides a method of providing an improved T cell redirection therapy to a subject in need thereof, comprising: administering to the subject an isolated molecule comprising a first polypeptide, a second polypeptide and a third polypeptide, wherein the first polypeptide comprises, from N- to C-terminus, a VH that is capable of specifically CD8, a CH1 domain, a hinge, a CH2 domain, a CH3 domain and a second antigen binding domain comprising a scFv that specifically binds a TCR complex; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure provides a method of targeting CD8⁺ CTLs to an undesired cell in a subject, comprising administering to the subject an isolated molecule comprising a first antigen binding domain, a second antigen binding domain and a third antigen binding domain, wherein the first antigen binding domain specifically binds CD8, the second antigen binding domain specifically binds a TCR complex and the third antigen binding domain specifically binds an antigen expressed by the undesired cell, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure also provides a method of targeting CD8⁺ CTLs to an undesired cell in a subject, comprising administering to the subject an isolated molecule comprising a first polypeptide, a second polypeptide and a third polypeptide, wherein the first polypeptide comprises, from N- to C-terminus, a second antigen binding domain comprising a scFv that specifically binds a TCR complex, a VH that is capable of specifically binding CD8, a CH1 domain, a hinge, a CH2 domain and a CH3 domain; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by the undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure also provides a method of targeting CD8⁺ CTLs to an undesired cell in a subject, comprising administering to the subject an isolated molecule comprising a first polypeptide, a second polypeptide and a third polypeptide, wherein the first polypeptide comprises, from N- to C-terminus, a VH that is capable of specifically binding CD8, a CH1 domain, a hinge, a CH2 domain and a CH3 domain; the second polypeptide comprises, from N- to C-terminus, a VL that is capable of specifically binding CD8, a CL domain and a second antigen binding domain comprising a scFv that specifically binds a TCR complex; and the third polypeptide comprises, from N- to C-terminus, a third antigen binding domain comprising a scFv that specifically binds an antigen expressed by the undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8

In yet another aspect, the disclosure also provides a method of targeting CD8⁺ CTLs to an undesired cell in a subject, comprising administering to the subject an isolated molecule comprising a first polypeptide, a second polypeptide and a third polypeptide, wherein the first polypeptide comprises, from N- to C-terminus, a VH that is capable of specifically CD8, a CH1 domain, a hinge, a CH2 domain, a CH3 domain and a second antigen binding domain comprising a scFv that specifically binds a TCR complex; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by the undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure provides a method of treating a cancer in a subject, comprising: administering to the subject an isolated molecule comprising a first antigen binding domain, a second antigen binding domain and a third antigen binding domain, wherein the first antigen binding domain specifically binds CD8, the second antigen binding domain specifically binds a TCR complex and the third antigen binding domain specifically binds an antigen expressed by an undesired cell, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure also provides a method of treating a cancer in a subject, comprising administering to the subject an isolated molecule comprising a first polypeptide, a second polypeptide and a third polypeptide, wherein the first polypeptide comprises, from N- to C-terminus, a second antigen binding domain comprising a scFv that specifically binds a TCR complex, a VH that is capable of specifically binding CD8, a CH1 domain, a hinge, a CH2 domain and a CH3 domain; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure also provides a method of treating a cancer in a subject, comprising administering to the subject an isolated molecule comprising a first polypeptide, a second polypeptide and a third polypeptide, wherein the first polypeptide comprises, from N- to C-terminus, a VH that is capable of specifically binding CD8, a CH1 domain, a hinge, a CH2 domain and a CH3 domain; the second polypeptide comprises, from N- to C-terminus, a VL that is capable of specifically binding CD8, a CL domain and a second antigen binding domain comprising a scFv that specifically binds a TCR complex; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure also provides a method of treating a cancer in a subject, comprising administering to the subject an isolated molecule comprising a first polypeptide, a second polypeptide and a third polypeptide, wherein the first polypeptide comprises, from N- to C-terminus, a VH that is capable of specifically CD8, a CH1 domain, a hinge, a CH2 domain, a CH3 domain and a second antigen binding domain comprising a scFv that specifically binds a TCR complex; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure provides a method of enhancing a CD8⁺ CTL response against an undesired cell in a subject, comprising: administering to the subject an isolated molecule comprising a first antigen binding domain, a second antigen binding domain and a third antigen binding domain, wherein the first antigen binding domain specifically binds CD8, the second antigen binding domain specifically binds a TCR complex and the third antigen binding domain specifically binds an antigen expressed by an undesired cell, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure also provides a method of enhancing a CD8⁺ CTL response against an undesired cell in a subject, comprising administering to the subject an isolated molecule comprising a first polypeptide, a second polypeptide and a third polypeptide, wherein the first polypeptide comprises, from N- to C-terminus, a second antigen binding domain comprising a scFv that specifically binds a TCR complex, a VH that is capable of specifically binding CD8, a CH1 domain, a hinge, a CH2 domain and a CH3 domain; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure also provides a method of enhancing a CD8⁺ CTL response against an undesired cell in a subject, comprising administering to the subject an isolated molecule comprising a first polypeptide, a second polypeptide and a third polypeptide, wherein the first polypeptide comprises, from N- to C-terminus, a VH that is capable of specifically binding CD8, a CH1 domain, a hinge, a CH2 domain and a CH3 domain; the second polypeptide comprises, from N- to C-terminus, a VL that is capable of specifically binding CD8, a CL domain and a second antigen binding domain comprising a scFv that specifically binds a TCR complex; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In yet another aspect, the disclosure also provides a method of enhancing a CD8⁺ CTL response against an undesired cell in a subject, comprising administering to the subject an isolated molecule comprising a first polypeptide, a second polypeptide and a third polypeptide, wherein the first polypeptide comprises, from N- to C-terminus, a VH that is capable of specifically CD9, a CH1 domain, a hinge, a CH2 domain, a CH3 domain and a second antigen binding domain comprising a scFv that specifically binds a TCR complex; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In some embodiments, the subject has a cancer, an infection, or an immune-mediated disease. In some embodiments, the cancer is a hematological malignancy or a solid tumor. In some embodiments, the hematological malignancy comprises acute lymphoblastic leukemia, acute myeloid leukemia, anaplastic large-cell lymphoma, Burkitt's lymphoma, chronic lymphocytic leukemia, chronic myeloid leukemia, diffuse large B-cell lymphoma, dendritic cell neoplasm, follicular lymphoma, hairy cell leukemia, Hodgkin's lymphoma, leukemia, B cell leukemia, T cell leukemia, light chain amyloidosis, lymphoma, B cell lymphoma, NK cell lymphoma, T cell lymphoma, mantle-cell lymphoma, marginal zone B-cell lymphoma, monoclonal gammopathy of undetermined significance, mucosa-associated lymphatic tissue lymphoma, multiple myeloma, myelodysplastic syndrome, non-Hodgkin's lymphoma, plasma cell leukemia, precursor B-cell lymphoblastic leukemia, smoldering multiple myeloma, Waldenstrom's macroglobulinemia, B cell malignancy, T cell malignancy, NK cell malignancy, or any combination thereof.

In some embodiments, the solid tumor comprises adenocarcinoma, anal cancer, basal cell carcinoma, biliary tract cancer, bladder cancer, bone cancer, breast cancer, cancer associated with infection, cancer of the adrenal gland, cancer of the endocrine system, cancer of the head or neck, cancer of the parathyroid gland, cancer of the penis, cancer of the thyroid gland, cancer of the urethra, cervical cancer, carcinoma of the breast, carcinoma of the fallopian tubes, carcinoma of the liver, carcinoma of the lung, carcinoma of the prostate, carcinoma of the renal pelvis, carcinoma of the vagina, carcinoma of the vulva, choriocarcinoma, clear cell carcinoma, colon cancer, colon carcinoma, colorectal cancer, connective tissue cancer, cutaneous or intraocular malignant melanoma, environmentally induced cancer, gastric cancer, gastrointestinal cancer, glioma, glioblastoma, endometrial cancer, epithelial cancer, esophageal cancer, eye cancer, larynx cancer, liver cancer, hepatocellular carcinoma, hormone refractory prostate adenocarcinoma, Kaposi's sarcoma, kidney cancer, lung cancer gastro-esophageal cancer, melanoma, mesothelioma, Merkel cell cancer, neuroblastoma, non-small cell lung cancer (NSCLC), osteosarcoma, ovarian cancer, pancreatic cancer, prostate cancer, rectal cancer, renal cell carcinoma, retinoblastoma rhabdomyosarcoma, squamous cell cancer, soft tissue sarcoma, solid tumors of childhood, spinal axis tumor, stomach cancer, testicular cancer, thyroid cancer, uterine cancer, urothelial carcinoma or sarcomas, or any combination thereof.

In some embodiments, the infection comprises infection with adenovirus, arboviral encephalitis virus, coronavirus, coxsackie virus, cytomegalovirus (CMV), dengue virus, echovirus, Epstein Barr virus, flaviviruses, human immunodeficiency virus (HIV), hepatitis A virus, hepatitis B virus, hepatitis C virus, herpes virus, HTLV virus, influenza virus, JC virus, measles virus, molluscum virus, mumps virus, papillomavirus, parvovirus, poliovirus, rabies virus, respiratory syncytial virus, rhinovirus, rotavirus, rubella virus or vaccinia virus, bacteria, virus, fungi, protozoa, parasite or prion, or any combination thereof.

In some embodiments, the immune-mediated disease comprises systemic lupus erythematosus (SLE), ankylosing spondylitis, Chagas disease, chronic obstructive pulmonary disease, Crohn's Disease, dermatomyositis, diabetes mellitus type 1, endometriosis, Goodpasture's syndrome, Graves' disease, Guillain-Barre syndrome (GBS), Hashimoto's disease, hidradenitis suppurativa, Kawasaki disease, IgA nephropathy, idiopathic thrombocytopenic purpura, interstitial cystitis, mixed connective tissue disease, morphea, multiple sclerosis, myasthenia gravis, narcolepsy, neuromyotonia, pemphigus vulgaris, pernicious anaemia, psoriasis, psoriatic arthritis, polymyositis, primary biliary cirrhosis, relapsing polychondritis, rheumatoid arthritis (RA), sarcoidosis, schizophrenia, scleroderma, Sjogren's syndrome, temporal arteritis, ulcerative colitis, vasculitis, vitiligo, Wegener's granulomatosis, IgG4-related disease, anti-synthetase syndrome, and autoimmunity associated with immunodeficiency including chronic variable immunodeficiency, Wiskott-Aldrich syndrome, Good syndrome, IgA deficiency, Hyper IgM syndrome, complement disorders, seropositive RA, SLE, postmyocardial infarction syndrome, subacute bacterial endocarditis, anti-glomerular basement membrane nephritis, autoimmune hepatitis, primary biliary cirrhosis, alopecia areata, bullous pemphigoid, cicatricial pemphigoid, dermatitis herpetiformis, gestational pemphigoid, pemphigus vulgaris, systemic scleroderma, Addison's disease, autoimmune polyendocrine syndrome type 2, autoimmune pancreatitis, diabetes mellitus type 1, autoimmune thyroiditis, Graves' disease, Sjogren's syndrome, celiac disease, antiphospholipid syndrome, autoimmune thrombocytopenic purpura, cold agglutinin disease, pernicious anemia, thrombocytopenia, adult onset Still's disease, CREST syndrome, drug-induced lupus, enthesitis-related arthritis, juvenile arthritis, mixed connective tissue disease, palindromic rheumatism, Parry Romberg syndrome, rheumatic fever, undifferentiated connective tissue disease, dermatomysitis, myasthenia gravis, neuromyotonia, paraneoplastic cerebellar degeneration, polymyositis, Bickerstaffs encephalitis, chronic inflammatory demyelinating polyneuropathy, Guillain-Barre syndrome, Hashimoto's encephalopathy, Lambert-Eaton myasthenic syndrome, multiple sclerosis, progressive inflammatory neuropathy, Stiff person syndrome, autoimmune uveitis, neuromyelitis optica, symphathetic ophthalmia, Meniere's disease, anti-neutrophil cytoplasmic antibody-associated vasculitis, Churg-Strauss syndrome, Henoch-Schonlein purpura, microscopic polyangiitis, urticarial vasculitis, and vasculitis. Examples of autoantibody-associated autoimmune conditions include gastritis and POEMS syndrome. Examples of autoantibody-associated (non-autoimmune) diseases include agammaglobulinemia, amyotrophic lateral sclerosis, Castleman's disease, cutaneous leukocytoclastic angiitis, eczema, eosinophilic gastroenteritis, erythroblastosis fetalis, fibrodysplasia ossificans progressive, hypogammaglobulinemia, idiopathic pulmonary fibrosis, IgA nephropathy, Majeed syndrome, narcolepsy, Rasmussen's encephalitis, spondyloarthropathy or Sweet's syndrome, or any combination thereof.

In yet another aspect, the disclosure provides a system comprising a means for selective activation or recruitment of CD8⁺ CTLs.

In yet another aspect, the disclosure also provides a composition comprising an antibody comprising a first antigen binding domain and a second antigen binding domain, and means for selective activation or recruitment of CD8⁺ CTLs.

In yet another aspect, the disclosure also provides a composition for enhancing an immune response against an antigen expressed by an undesired cell, comprising means for selective activation or recruitment of CD8⁺ CTLs.

In yet another aspect, the disclosure also provides a composition for treating a cancer in subject, comprising means for selective activation or recruitment of CD8⁺ CTLs.

In yet another aspect, the disclosure also provides a system comprising a means for providing an improved T cell redirecting therapeutic treatment to a subject.

In yet another aspect, the disclosure also provides a T cell redirecting therapeutic comprising a means for improving safety of the T cell redirecting therapeutic.

In yet another aspect, the disclosure also provides a process for generating an improved T cell redirecting therapeutic, comprising: a step for performing a function of designing the T cell redirecting therapeutic comprising the means of the disclosure; and a step for performing a function of producing the T cell redirecting therapeutic comprising the means of the disclosure.

In yet another aspect, the disclosure provides a method of isolating, separating, purifying, sorting, selecting or capturing a CD8+ CTL comprising: providing a sample comprising the CD8+ CTL; contacting the sample with an isolated molecule comprising a first antigen binding domain and a second antigen binding domain, wherein the first antigen binding domain specifically binds CD8 and the second antigen binding domain specifically binds a TCR complex; and isolating, separating, purifying, sorting, selecting or capturing the CD8⁺ CTL bound to the isolated molecule.

In yet another aspect, the disclosure also provides a method of isolating, separating, purifying, sorting, selecting or capturing a CD8+ CTL, comprising contacting the CD8+ CTL with an isolated molecule comprising a first antigen binding domain and a second antigen binding domain, wherein the first antigen binding domain specifically binds CD8 and the second antigen binding domain specifically binds a TCR complex; and isolating, separating, purifying, sorting, selecting or capturing the CD8+ CTL based on binding of the CD8+ CTL to the isolated molecule.

### BRIEF DESCRIPTION OF THE DRAWINGS

FIG. 1 shows the design of the Protein Format 1. In the Protein Format 1, the tumor associated antigen (TAA) binding arm was incorporated as a scFv coupled to a Fc (HC1_scFv), the CD8 binding arm was incorporated as a HC/LC chain (HC2 N-term and LC2 2nd N-term), and the CD3 binding arm was incorporated as a scFv attached to the N-terminus of the CD8 binding HC (LC2 1st N-term).

FIG. 2 shows the design of the Protein Format 2. In the Protein Format 2, the TAA binding arm was incorporated as a scFv coupled to the Fc (HC1_scFv), the CD8 binding arm was incorporated as a HC/LC chain (HC2 N-term and LC2 1st N-term), and the CD3 binding arm was incorporated as a scFv attached to the C-terminus of the CD8 binding LC (LC2 C-term).

FIG. 3 shows the design of the Protein Format 3. In the Protein Format 3, the TAA binding arm was incorporated as a scFv coupled to the Fc (HC1_scFv), the CD8 binding arm was incorporated as a HC/LC chain (HC2 N-term and LC1 1st N-term), and the CD3 binding arm was incorporated as a scFv attached to the C-terminus of the CD8 binding HC (HC2 C-term).

FIG. 4A-4B show low affinity CD3 multispecifics paired with CD8 binders show selective binding to CD8 T cells. FIG. 4A shows that the trispecific binds to and specifically recruits CD8 T cells. FIG. 4B shows that Pan T cells were isolated from the PBMCs of healthy volunteers and stained with the test multispecifics at room temperature for 30 min followed by detection using an anti-human IgG antibody and staining with anti-human CD3, CD4 and CD8 antibodies. % binding was determined using the secondary antibody-stained samples as negative controls.

FIG. 5A shows in the top panel cytotoxicity assay on C4-2B (target) and PBMCs (effector) at 3 different E:T ratios incubated for 72 h in the presence of CD8×CD3×PSMA trispecific Ab (black circle), CD8×PSMA bispecific Ab (black square) and CD3×PSMA bispecific Ab (grey triangle). EC50 values listed in the table are for the CD8×CD3×PSMA trispecific Ab (CD8B573.001). The low panel in FIG. 5A shows cytotoxicity assay on C4-2B (target) and PBMCs (effector) with E:T ratio of 3:1 and incubated for 72 h (left) and 48 h (right) in the presence of indicated Ab. Table list EC50 values for CD3×CD8×PSMA (low affinity CD3), CD3×PSMA (CD8B52, CD3B376) [medium affinity CD3], CD3×PSMA (CD3B220, HA) [high affinity CD3].

FIG. 5B shows the IncuCyte cytotoxicity assay on target cell line C4-2B and PBMCs (2 donors: 19054280 and 19053791) in the presence of indicated Ab ranging from 0 (NBS) to 60 nM.

FIG. 6 shows low affinity CD3 multispecifics paired with CD8 binders show potent cytotoxicity against target cell lines in a CD8 T cell dependent manner. PBMCs of healthy volunteers were either depleted of CD8 T cells or used as such. CD8 depleted and non depleted PBMCs were cocultured with C4-2B target cells as a 1:1 effector to target ratio (CD3 to target cells) for 72 hrs in the presence of the test multispecifics. Cytotoxicity was monitored using the Incucyte automated live cell analysis system and EC50 values were calculated after normalizing to no multispecific containing wells.

FIG. 7 shows low affinity CD3 multispecifics paired with CD8 binders specifically and potently activate only CD8 T cells. PBMCs were cocultured with C4-2B target cells as a 1:1 effector to target ratio (CD3 to target cells) for the indicated time points in the presence of the test multispecifics. At each time point, cells were harvested and CD3, CD4 and CD8 T cells were analyzed for the presence of the indicated activation and exhaustion markers.

FIG. 8 shows low affinity CD3 multispecifics paired with CD8 binders show reduced anti-inflammatory cytokine release. PBMCs were cocultured with C4-2B target cells as a 1:1 effector to target ratio (CD3 to target cells) for the indicated time points in the presence of the test multispecifics. At each time point, supernatants were harvested and analyzed for the indicated cytokines using a multiplex Luminex analysis system.

### DETAILED DESCRIPTION

The disclosed methods may be understood more readily by reference to the following detailed description taken in connection with the accompanying Figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed compositions or methods.

All patents, published patent applications and publications cited herein are incorporated by reference as if set forth fully herein.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The transitional terms “comprising,” “consisting essentially of,” and “consisting of” are intended to connote their generally accepted meaning, that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Activate” or “activation” or “activated” refers to induction of a change in the biologic state of a cell resulting in expression of activation markers, cytokine production, proliferation or mediating cytotoxicity of target cells. Cells may be activated by primary stimulatory signals. Co-stimulatory signals may amplify the magnitude of the primary signals and suppress cell death following initial stimulation resulting in a more durable activation state and thus a higher cytotoxic capacity. An exemplary activated cell is an activated CD8⁺ CTL that expresses CD25 and/or produces cytokines such as IFNγ.

“Affinity” or “binding affinity” or “binds with affinity” refers to the strength of the sum total of noncovalent interactions between a single binding site of a molecule (such as molecules and multispecific antibodies described herein) and its binding partner (i.e., an antigen). Unless indicated otherwise, “affinity” refers to intrinsic binding affinity which reflects a 1:1 interaction between members of a binding pair. The affinity can generally be represented by the dissociation constant (K_{D}). Affinity can be measured by known methods, such as using biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kon” and an “of-rate” or “rate of dissociation” or “dissociation rate” or “koff” may also be determined with the same methods. “High affinity” within the context of this disclosure refers to molecules which demonstrate stronger binding to an antigen (e.g., lower K_{D}). “Low affinity” within the context of this disclosure refers to molecules which demonstrate weaker binding to an antigen (e.g., higher K_{D}).

“Non-antibody scaffold” refers to a single chain protein framework that contains a structured core associated with variable domains of high conformational tolerance. The variable domains tolerate variation to be introduced without compromising scaffold integrity, and hence the variable domains can be engineered and selected for binding to a specific antigen.

“Antigen” refers to any molecule (e.g., protein, peptide, polysaccharide, glycoprotein, glycolipid, nucleic acid, portions thereof, or combinations thereof) that is capable of mediating an immune response either alone or in complex in MHC. Exemplary immune responses include antibody production and activation of immune cells, such as T cells, B cells or NK cells. Antigens may be expressed by genes, synthetized, or purified from biological samples such as a tissue sample, a tumor sample, a cell or a fluid with other biological components, organisms, subunits of proteins/antigens, killed or inactivated whole cells or lysates.

“Antigen binding domain” or “antigen binding fragment” or “domain that binds an antigen” refers to a portion of a molecule that specifically binds an antigen. Antigen binding domain may include portions of an immunoglobulin that bind an antigen, such as a VH, a VL, the VH and the VL, Fab, Fab′, F(ab′)2, Fd and Fv fragments, domain antibodies (dAb) consisting of one VH or one VL, shark variable IgNAR domains, camelized VH domains, VHH, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3 and non-antibody scaffolds that bind an antigen.

“Antibodies” is meant in a broad sense and includes immunoglobulin molecules including monoclonal antibodies including murine, human, humanized and chimeric monoclonal antibodies, antigen binding domains, multispecific antibodies, such as bispecific, trispecific, tetraspecific, dimeric, trimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Bispecific” refers to a molecule that specifically binds two distinct antigens or two distinct epitopes within the same antigen. The bispecific molecule may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

“Cancer cell” or “tumor cell” refers to a cancerous, pre-cancerous or transformed cell, either in vivo, ex vivo, or in tissue culture, that has spontaneous or induced phenotypic changes. Cancer cells may exhibit characteristics such as morphological changes, immortalization, aberrant growth, foci formation, proliferation, malignancy, modulation of tumor specific marker levels or invasiveness.

“CH2 domain” or “CH2 region” refers to the CH2 region of an immunoglobulin. The CH2 region of a human IgG1 antibody corresponds to amino acid residues 231-340 (EU numbering) of IgG1 constant domain. The amino acid sequence of a wild-type IGG1 CH2 domain is shown in SEQ ID NO: 2318.

(IgG1 CH2)
SEQ ID NO: 2318
APELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNK
ALPAPIEKTISKA

“CH3 domain” or “CH3 region” refers to the CH3 region of an immunoglobulin. The CH3 region of human IgG1 antibody corresponds to amino acid residues 341-446 (EU numbering) of IgG1 constant domain. The amino acid sequence of a wild-type IgG1 CH3 domain is shown in SEQ ID NO: 2319.

SEQ ID NO: 2319
GQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHY
TQKSLSLSPGK

“CD3ε” refers to CD3ε from any species, such as from primate or rodent, such as human, monkey, rat or mouse. Human CD3ε comprises the amino acid sequence of SEQ ID NO: 2180.

(CD3ϵ)
SEQ ID NO: 2180
DGNEEMGGITQTPYKVSISGTTVILTCPQYPGSEILWQHNDKNIGGDE
DDKNIGSDEDHLSLKEFSELEQSGYYVCYPRGSKPEDANFYLYLRARV
CENCMEMDVMSVATIVIVDICITGGLLLLVYYWSKNRKAKAKPVTRGA
GAGGRQRGQNKERPPPVPNPDYEPIRKGQRDLYSGLNQRRI

“CD8” refers to CD8 from any species, such as from primate or rodent, such as human, monkey, rat or mouse. Human CD8 is a homodimer of alpha chains (CD8a) or a heterodimer of CD8α (SEQ ID NO: 2181) and CD8β (SEQ ID NO: 2182) chains.

(CD8α chain)
SEQ ID NO: 2181
SQFRVSPLDRTWNLGETVELKCQVLLSNPTSGCSWLFQPRGAAASPTF
LLYLSQNKPKAAEGLDTQRFSGKRLGDTFVLTLSDFRRENEGYYFCSA
LSNSIMYFSHFVPVFLPAKPTTTPAPRPPTPAPTIASQPLSLRPEACR
PAAGGAVHTRGLDFACDIYIWAPLAGTCGVLLLSLVITLYCNHRNRRR
VCKCPRPVVKSGDKPSLSARYV
(CD8β chain)
SEQ ID NO: 2182
LQQTPAYIKVQTNKMVMLSCEAKISLSNMRIYWLRQRQAPSSDSHHEF
LALWDSAKGTIHGEEVEQEKIAVFRDASRFILNLTSVKPEDSGIYFCM
IVGSPELTFGKGTQLSVVDFLPTTAQPTKKSTLKKRVCRLPRPETQKG
PLCSPITLGLLVAGVLVLLVSLGVAIHLCCRRRRARLRFMKQFYK

“Complementarity determining regions” (CDR) are regions of an antibody that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al. (1970) J Exp Med 132: 211-50; Kabat et al., Sequences of Proteins of Immunological Interest, 5th Ed. Public Health Service, National Institutes of Health, Bethesda, Md., 1991; Kabat et al., J. Biol. Chem. 252:6609-6616 (1977); Kabat, Adv. Prot. Chem. 32:1-75 (1978)), Chothia (Chothia et al. (1987) J Mol Biol 196: 901-17), IMGT (Lefranc et al. (2003) Dev Comp Immunol 27: 55-77). Both terminologies are well recognized in the art. CDR region sequences have also been defined by AbM, AbM (Martin and Thornton J Bmol Biol 263: 800-15, 1996), Contact and IMGT. The correspondence between the various delineations and variable region numbering is described (see e.g., Lefranc et al. (2003) Dev Comp Immunol 27: 55-77; Honegger and Pluckthun, J Mol Biol (2001) 309:657-70; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT, AbM or Contact, unless otherwise explicitly stated in the specification.

The light chain variable region CDR1 domain is interchangeably referred to herein as LCDR1 or VL CDR1. The light chain variable region CDR2 domain is interchangeably referred to herein as LCDR2 or VL CDR2. The light chain variable region CDR3 domain is interchangeably referred to herein as LCDR3 or VL CDR3. The heavy chain variable region CDR1 domain is interchangeably referred to herein as HCDR1 or VH CDR1. The heavy chain variable region CDR2 domain is interchangeably referred to herein as HCDR2 or VH CDR2. The heavy chain variable region CDR1 domain is interchangeably referred to herein as HCDR3 or VH CDR3.

Exemplary CDR region sequences are illustrated herein, for example, in the tables provided in the Examples below. The positions of CDRs within a canonical antibody variable region have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra (1997)). Such nomenclature is similarly well known to those skilled in the art.

The term “hypervariable region”, such as a VH or VL, when used herein refers to the regions of an antibody variable region that are hypervariable in sequence and/or form structurally defined loops. Generally, antibodies comprise six hypervariable regions; three in the VH (HCDR1, HCDR2, HCDR3), and three in the VL (LCDR1, LCDR2, LCDR3). A number of hypervariable region delineations are in use and are encompassed herein. The “Kabat” CDRs are based on sequence variability and are the most commonly used (see, e.g., Kabat et al., Sequences of Proteins of Immunological Interest, 5th Ed. Public Health Service, National Institutes of Health, Bethesda, Md. (1991)). “Chothia” refers instead to the location of the structural loops (see, e.g., Chothia and Lesk, J. Mol. Biol. 196:901-917 (1987)). The end of the Chothia CDR-HCDR1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The “AbM” hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). “Contact” hypervariable regions are based on an analysis of the available complex crystal structures.

Recently, a universal numbering system has been developed and widely adopted, ImMunoGeneTics (IMGT) Information System® (Lafranc et al., Dev. Comp. Immunol. 27(1):55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). An Exemplary system, shown herein, combines Kabat and Chothia.

| | Exemplary | IMGT | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions may comprise “extended hypervariable regions” as follows: 24-36 or 24-34 (LCDR1), 46-56 or 50-56 (LCDR2) and 89-97 or 89-96 (LCDR3) in the VL and 26-35 or 26-35A (HCDR1), 50-65 or 49-65 (HCDR2) and 93-102, 94-102, or 95-102 (HCDR3) in the VH. CDR sequences, reflecting each of the above numbering schemes, are provided herein, including in the tables provided in the Examples below.

“Reduce” or “reduced” refers to a decrease in a measured response mediated by a test molecule in any system in vitro or in vivo when compared to a control. Measured response may be an Fc-mediated effector function such as ADCC, CDC and/or ADCP, cellular proliferation or activation, or cell killing. “Reduced” may be a reduction of about 10%, 20%, 30%, 40%, 50%, 60%, 70%, 80%, 90%, 100% or more, or a statistically significant reduction when compared to a control. Suitable controls depend on the assay or response and are known.

“Enhance” or “enhanced” refers to an increase in a measured response mediated by a test molecule in any system in vitro or in vivo when compared to a control. Measured response may be an Fc-mediated effector function such as ADCC, CDC and/or ADCP, cellular proliferation or activation, or cell killing. “Enhanced” may be an increase of about 10%, 20%, 30%, 40%, 50%, 60%, 70%, 80%, 90%, 100% or more, or a statistically significant increase when compared to a control. Suitable controls depend on the assay or response and are known.

“Domain antibody” or “dAb” refers to an antibody fragment composed of a VH domain.

“Fab” or “Fab fragment” refers to an antibody fragment composed of VH, CH1, VL and CL domains.

“F(ab′)₂” or “F(ab′)₂ fragment” refers to an antibody fragment containing two Fab fragments connected by a disulfide bridge in the hinge region.

“Fc” or “Fc region” or “Fc domain” refers to an antibody region comprising at least a portion of a hinge region, a CH2 domain and a CH3 domain. The Fc may be generated by digestion of an antibody with papain, or pepsin where the Fc is the fragment obtained thereby, which includes one or both CH2-CH3 domains of and a portion of the hinge region.

“Fd” or “Fd fragment” refers to an antibody fragment composed of VH and CH1 domains.

“Fv” or “Fv fragment” refers to an antibody fragment composed of the VH and the VL domains from a single arm of the antibody.

“Full length antibody” is comprised of two heavy chains (HC) and two light chains (LC) inter-connected by disulfide bonds as well as multimers thereof (e.g., IgM). Each heavy chain is comprised of a VH and a heavy chain constant domain, the heavy chain constant domain comprised of subdomains CH1, hinge, CH2 and CH3. Each light chain is comprised of a VL and a light chain constant domain (CL). The VH and the VL may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Half molecule”, in the context of an antibody that comprises two heavy chains of fragments thereof (such as two Fc regions), refers to one heavy chain or a fragment thereof and any additional polypeptides that associate with the one heavy chain or fragment thereof or are conjugated to the one heavy chain or fragment thereof. An exemplary half molecule is a molecule comprising a scFv conjugated to Fc. Another exemplary half molecule is a molecule comprising a HC conjugated to scFv.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice, rats or chicken carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some instances, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

The terms “identical” or percent “identity,” in the context of two or more nucleic acids or polypeptide sequences (e.g., CD8 antibody and polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

“Isolated” refers to a homogenous population of molecules (such as synthetic polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, trispecific, monovalent, bivalent, trivalent or multivalent.

“Multispecific” refers to a molecule that specifically binds two or more distinct antigens or two or more distinct epitopes within the same antigen. Multispecific molecule may have cross-reactivity to other related antigens, for example to the same antigen from other species (homologs), such as human or monkey, for example Macaca fascicularis (cynomolgus, cyno) or Pan troglodytes, or may bind an epitope that is shared between two or more distinct antigens.

“Molecule” refers to a protein that may be monomeric, multimeric, homodimeric or heterodimeric protein. Multimeric protein may be composed of two or more identical or distinct subunits. Trimeric protein is composed of three subunits which may be identical or distinct, or alternatively, two subunits may be identical and the third subunit distinct.

“Pharmaceutical composition” refers to a composition that results from combining an active ingredient and one or more pharmaceutically acceptable carriers.

“Pharmaceutically acceptable carrier” or “excipient” refers to an ingredient in a pharmaceutical composition, other than the active ingredient, which is nontoxic to a subject. Exemplary pharmaceutically acceptable carriers are a buffer, stabilizer or preservative.

“Prevent,” “preventing,” or “prophylaxis” of a disease or disorder means preventing that a disorder occurs in a subject.

“Protein” or “polypeptide” are used interchangeably herein are refers to a molecule that comprises one or more polypeptides each comprised of at least two amino acid residues linked by a peptide bond. Protein may be a monomer, or may be protein complex of two or more subunits, the subunits being identical or distinct. Small polypeptides of less than 50 amino acids may be referred to as “peptides”. Protein may be a heterologous fusion protein, a glycoprotein, or a protein modified by post-translational modifications such as phosphorylation, acetylation, myristoylation, palmitoylation, glycosylation, oxidation, formylation, amidation, citrullination, polyglutamylation, ADP-ribosylation, pegylation or biotinylation. Protein may be recombinantly expressed.

“Recombinant” refers to polynucleotides, polypeptides, vectors, viruses and other macromolecules that are prepared, expressed, created or isolated by recombinant means.

“Sample” refers to a collection of similar fluids, cells, or tissues isolated from a subject, as well as fluids, cells, or tissues present within a subject. Exemplary samples are biological fluids such as blood, serum and serosal fluids, plasma, lymph, urine, saliva, cystic fluid, tear drops, feces, sputum, mucosal secretions of the secretory tissues and organs, vaginal secretions, ascites fluids such as those associated with non-solid tumors, fluids of the pleural, pericardial, peritoneal, abdominal and other body cavities, fluids collected by bronchial lavage, liquid solutions contacted with a subject or biological source, for example, cell and organ culture medium including cell or organ conditioned medium, lavage fluids and the like, tissue biopsies, fine needle aspirations or surgically resected tumor tissue.

“Single chain Fv” or “scFv” refers to a fusion protein comprising a VH and a VL, which are optionally linked via a polypeptide linker. scFv may have the VL and VH variable regions in either order, e.g., with respect to the N-terminal and C-terminal ends of the polypeptide, the scFv may comprise VL-linker-VH or may comprise VH-linker-VL. scFv may comprise one or more disulfide bonds to stabilize the scFv.

“Specifically binds,” “specific binding,” “specifically binding” or “binds” refer to a molecule comprising an antigen binding domain that binds the antigen with greater affinity than other antigens. Typically, the molecule binds the antigen with a dissociation constant (K_{D}) of about 1×10⁻⁷ M or less, for example about 5×10⁻⁸ M or less, about 1×10⁻⁸M or less, about 1×10⁻⁹M or less, about 1×10⁻¹⁰ M or less, about 1×10⁻¹¹ M or less, or about 1×10⁻¹²M or less, typically with the K_{D} that is at least one hundred fold less than its K_{D} for binding to a non-specific antigen (e.g., BSA, casein).

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

“T cell receptor complex” (TCR complex) refers to a known TCR complex comprising of a TCRα and TCRβ chains, CD3ε, CD3γ, CD3δ and CD3ζ molecules. In some instances, TCRα and TCRβ chains are replaced by TCRγ and TCRζ chains. The amino acid sequences of the various proteins forming the TCR complex are well-known.

“Therapeutically effective amount” or “effective amount” used interchangeably herein, refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Example indicators of an effective therapeutic or combination of therapeutics that include, for example, improved wellbeing of the patient, reduction of a tumor burden, arrested or slowed growth of a tumor, and/or absence of metastasis of cancer cells to other locations in the body.

“Treat,” “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Trispecific” refers to a molecule that specifically binds three distinct antigens or three distinct epitopes within the same antigen. Trispecific molecule may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Unable to activate” in the context of CD8⁺ CTL activation refers to a molecule that exhibits no measurable activation of CD8⁺ CTLs in a system, such as in an in vitro assay. CD8⁺ CTL activation may be measured using known methods, such as assessing increased CD25 expression or by production IFNγ by the CD8⁺ CTL.

“Undesired cell” refers to a cell that is desired or intended to be removed from a system, such as an in vitro system an ex vivo system, a tissue, blood, sample, or from a subject.

“Expressed by an undesired cell” refers to a measurable intracellular or surface expression of an antigen by the undesired cell.

“VHH” refers to a single chain antigen binding domain derived from camelid antibodies which are devoid of light chains.

“BCMA” refers to B cell maturation antigen (TNFRSF17, CD269), a transmembrane protein belonging to the tumor necrosis family receptor (TNFR) superfamily that is primarily expressed on terminally differentiated B cells. BCMA expression is restricted to the B cell lineage and mainly present on plasma cells and plasmablasts and to some extent on memory B cells, but virtually absent on peripheral and naive B cells. BCMA is also expressed on multiple myeloma (MM) cells, on leukemia cells and lymphoma cells. The amino acid sequence of human BCMA is shown in SEQ ID NO: 2320. The extracellular domain spans residues 1-54, the transmembrane domain spans residues 55-77 and the cytoplasmic domain spans residues 78-184 of SEQ ID NO: 2320.

(BCMA)
SEQ ID NO: 2320
MLQMAGQCSQNEYFDSLLHACIPCQLRCSSNTPPLTCQRYCNASVTNS
VKGTNAILWTCLGLSLIISLAVFVLMFLLRKINSEPLKDEFKNTGSGL
LGMANIDLEKSRTGDEIILPRGLEYTVEECTCEDCIKSKPKVDSDHCF
PLPAMEEGATILVTTKTNDYCKSLPAALSATEIEKSISAR

“PSMA” refers to Prostate Specific Membrane Antigen. The amino acid sequence of the human PSMA is shown in SEQ ID NO: 2321. The extracellular domain spans residues 44-750, the transmembrane domain spans residues 20-43 and the cytoplasmic domain spans residues 1-19 of SEQ ID NO: 2321.

(PSMA)
SEQ ID NO: 2321
MWNLLHETDSAVATARRPRWLCAGALVLAGGFFLLGFLFGWFIKSSNE
ATNITPKHNMKAFLDELKAENIKKFLYNFTQIPHLAGTEQNFQLAKQI
QSQWKEFGLDSVELAHYDVLLSYPNKTHPNYISIINEDGNEIFNTSLF
EPPPPGYENVSDIVPPFSAFSPQGMPEGDLVYVNYARTEDFFKLERDM
KINCSGKIVIARYGKVFRGNKVKNAQLAGAKGVILYSDPADYFAPGVK
SYPDGWNLPGGGVQRGNILNLNGAGDPLTPGYPANEYAYRRGIAEAVG
LPSIPVHPIGYYDAQKLLEKMGGSAPPDSSWRGSLKVPYNVGPGFTGN
FSTQKVKMHIHSTNEVTRIYNVIGTLRGAVEPDRYVILGGHRDSWVFG
GIDPQSGAAVVHEIVRSFGTLKKEGWRPRRTILFASWDAEEFGLLGST
EWAEENSRLLQERGVAYINADSSIEGNYTLRVDCTPLMYSLVHNLTKE
LKSPDEGFEGKSLYESWTKKSPSPEFSGMPRISKLGSGNDFEVFFQRL
GIASGRARYTKNWETNKFSGYPLYHSVYETYELVEKFYDPMFKYHLTV
AQVRGGMVFELANSIVLPFDCRDYAVVLRKYADKIYSISMKHPQEMKT
YSVSFDSLFSAVKNFTEIASKFSERLQDFDKSNPIVLRMMNDQLMFLE
RAFIDPLGLPDRPFYRHVIYAPSSHNKYAGESFPGIYDALFDIESKVD
PSKAWGEVKRQIYVAAFTVQAAAETLSEVA

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, Md. (1991), unless otherwise explicitly stated. Various antibody numbering schemes are available at ImMunoGeneTics (IMGT) website via IMGT scientific charts.

Mutations in the Ig constant regions are referred to as follows: L351Y_F405A_Y407V refers to L351Y, F405A and Y407V mutations in an immunoglobulin chain. L351Y_F405A_Y407V/T394W refers to L351Y, F405A and Y407V mutations in a first immunoglobulin chain and T394W mutation in the second immunoglobulin chain in a heterodimeric molecule comprising both the first and the second immunoglobulin chains.

Compositions of Matter

The disclosure provides molecules having improved characteristics and functionality. The molecules of the disclosure selectively activate or recruit CD8⁺ CTLs without activating or recruiting non-CTL CD8 expressing cells. Without wishing to be bound by any particular theory, it is expected that the molecules of the disclosure provide a benefit in terms of therapeutic treatment when compared to other T cell redirecting molecules, mediating more efficient killing or undesired cells and exhibiting reduced side effect profile, particularly cytokine release syndrome observed with CD3 binding T cell redirecting molecules. The molecules of the disclosure may be utilized broadly to deplete or partially deplete any undesired cell, such as cancer cell, a virus infected cell, an immune cell, an inflamed cell, a damaged cell, a dysplastic cell, an immunogenic cell, a metaplastic cell or a mutant cell, or any combination thereof. The molecules of the disclosure therefore have utility across a spectrum of disease indications including cancer, infectious disease and immune-mediated diseases. The molecules of the disclosure have been designed in a manner that co-engagement of CD8 and CD3 is needed for activation and/or recruitment of the CD8⁺ CTLs. The molecules of the disclosure may be used to treat any mammalian or non-mammalian subject. The molecules of the disclosure may also be used to isolate, separate, purify, sort, select or capture CD8⁺ CTLs.

The disclosure provides an isolated molecule, comprising: a first antigen binding domain and a second antigen binding domain, wherein the first antigen binding domain specifically binds CD8 and the second antigen binding domain specifically binds a TCR complex.

The disclosure also provides an isolated molecule, comprising: a first antigen binding domain, a second antigen binding domain and a third antigen binding domain, wherein the first antigen binding domain specifically binds CD8, the second antigen binding domain specifically binds a TCR complex, and the third antigen binding domain specifically binds a third antigen.

In some embodiments, the third antigen comprises an antigen expressed by an undesired cell.

The disclosure also provides an isolated molecule, comprising: a first antigen binding domain, a second antigen binding domain and a third antigen binding domain, wherein the first antigen binding domain specifically binds CD8, the second antigen binding domain specifically binds a TCR complex, and the third antigen binding domain specifically binds an antigen expressed by an undesired cell.

The disclosure also provides an isolated molecule, comprising: a first antigen binding domain, a second antigen binding domain and a third antigen binding domain, wherein the first antigen binding domain specifically binds CD8, the second antigen binding domain specifically binds a TCR complex, and the third antigen binding domain specifically binds an antigen expressed by an undesired cell, wherein the isolated molecule activates or recruits CD8⁺ CTLs upon co-engagement of the TCR complex and CD8.

The disclosure also provides an isolated molecule, comprising: a first antigen binding domain, a second antigen binding domain and a third antigen binding domain, wherein the first antigen binding domain specifically binds CD8, the second antigen binding domain specifically binds a TCR complex, and the third antigen binding domain specifically binds an antigen expressed by an undesired cell, wherein the isolated molecule activates or recruits CD8⁺ CTLs upon co-engagement of the TCR complex and CD8 and is unable to activate or recruit CD8⁺ CTLs in the absence of co-engagement of the TCR complex and CD8.

The disclosure also provides an isolated molecule, comprising: a first antigen binding domain, a second antigen binding domain and a third antigen binding domain, wherein the first antigen binding domain specifically binds CD8, the second antigen binding domain specifically binds a TCR complex, and the third antigen binding domain specifically binds an antigen expressed by an undesired cell, wherein the first antigen binding domain specifically binds CD8 and the second antigen binding domain specifically binds the TCR with affinities that result in activation or recruitment of CD8⁺ CTLs only upon co-engagement of the TCR complex and CD8.

In some embodiments, the isolated molecule is an isolated antibody.

In some embodiments, the isolated molecule is based on one or more non-antibody scaffolds.

The disclosure also provides an isolated multispecific antibody, comprising: a first antigen binding domain and a second antigen binding domain, wherein the first antigen binding domain specifically binds CD8 and the second antigen binding domain specifically binds a TCR complex.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds a TCR complex, and the third antigen binding domain specifically binds a third antigen.

In some embodiments, the third antigen comprises an antigen expressed by an undesired cell.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds a TCR complex, and the third antigen binding domain specifically binds an antigen expressed by an undesired cell.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds a TCR complex, and the third antigen binding domain specifically binds an antigen expressed by an undesired cell, wherein the isolated multispecific antibody activates or recruits CD8⁺ CTLs upon co-engagement of the TCR complex and CD8.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds a TCR complex, and the third antigen binding domain specifically binds an antigen expressed by an undesired cell, wherein the isolated multispecific antibody activates or recruits CD8⁺ CTLs upon co-engagement of the TCR complex and CD8 and wherein the isolated multispecific antibody is unable to activate or recruit CD8⁺ CTLs in the absence of co-engagement of the TCR complex and CD8.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds a TCR complex, and the third antigen binding domain specifically binds an antigen expressed by an undesired cell, wherein the first antigen binding domain specifically binds CD8 and the second antigen binding domain specifically binds the TCR complex with affinities that result in activation or recruitment of CD8⁺ CTLs only upon co-engagement of the TCR complex and CD8.

The affinities (e.g., binding affinities) with which the isolated molecules or isolated multispecific antibodies of the disclosure bind to the various antigens are expressed as dissociation constants (K_{D}).

In some embodiments, the first antigen binding domain specifically binds CD8 with the K_{D} of about 0.1×10⁻⁹ M or higher, such as about 0.2×10⁻⁹ M or higher, about 0.3×10⁻⁹ M or higher, about 0.4×10⁻⁹ M or higher, about 0.5×10⁻⁹ M or higher, about 0.6×10⁻⁹ M or higher, about 0.7×10⁻⁹ M or higher, about 0.8×10⁻⁹ M or higher, about 0.9×10⁻⁹ M or higher, 1×10⁻⁹ M or higher, about 2×10⁻⁹ M or higher, about 3×10⁻⁹ M or higher, about 4×10⁻⁹ M or higher, about 5×10⁻⁹ M or higher, about 6×10⁻⁹ M or higher, about 7×10⁻⁹ M or higher, about 8×10⁻⁹ M or higher, about 9×10⁻⁹ M or higher, about 10×10⁻⁹ M or higher, about 15×10⁻⁹ M or higher, about 20×10⁻⁹ M or higher, about 25×10⁻⁹ M or higher, about 30×10⁻⁹ M or higher, about 35×10⁻⁹ M or higher, about 40×10⁻⁹ M or higher, about 45×10⁻⁹ M or higher, 50×10⁻⁹ M or higher, about 55×10⁻⁹ M or higher, about 60×10⁻⁹ M or higher, about 65×10⁻⁹ M or higher, about 70×10⁻⁹ M or higher, about 75×10⁻⁹ M or higher, about 80×10⁻⁹ M or higher, about 85×10⁻⁹ M or higher, about 90×10⁻⁹ M or higher, about 95×10⁻⁹ M or higher, about 100×10⁻⁹ M or higher, about 110×10⁻⁹ M or higher, about 120×10⁻⁹ M or higher, about 130×10⁻⁹ M or higher, about 140×10⁻⁹ M or higher, about 150×10⁻⁹ M or higher, about 160×10⁻⁹ M or higher, about 170×10⁻⁹ M or higher, about 180×10⁻⁹ M or higher, about 190×10⁻⁹ M or higher, about 200×10⁻⁹ M or higher, about 210×10⁻⁹ M or higher, about 220×10⁻⁹ M or higher, about 230×10⁻⁹ M or higher, about 240×10⁻⁹ M or higher, about 250×10⁻⁹ M or higher, about 260×10⁻⁹ M or higher, about 270×10⁻⁹ M or higher, about 280×10⁻⁹ M or higher, about 290×10⁻⁹ M or higher, about 300×10⁻⁹ M or higher, about 310×10⁻⁹ M or higher, about 320×10⁻⁹ M or higher, about 330×10⁻⁹ M or higher, about 340×10⁻⁹ M or higher, about 350×10⁻⁹ M or higher, about 360×10⁻⁹ M or higher, about 370×10⁻⁹ M or higher, about 380×10⁻⁹ M or higher, about 390×10⁻⁹ M or higher, about 400×10⁻⁹ M or higher, about 410×10⁻⁹ M or higher, about 420×10⁻⁹ M or higher, about 430×10⁻⁹ M or higher, about 440×10⁻⁹ M or higher, about 450×10⁻⁹ M or higher, about 460×10⁻⁹ M or higher, about 470×10⁻⁹ M or higher, about 480×10⁻⁹ M or higher, about 490×10⁻⁹ M or higher, about 400×10⁻⁹ M or higher, about 510×10⁻⁹ M or higher, about 520×10⁻⁹ M or higher, about 530×10⁻⁹ M or higher, about 540×10⁻⁹ M or higher, about 550×10⁻⁹ M or higher, about 560×10⁻⁹ M or higher, about 570×10⁻⁹ M or higher, about 580×10⁻⁹ M or higher, about 590×10⁻⁹ M or higher, about 600×10⁻⁹ M or higher, about 610×10⁻⁹ M or higher, about 620×10⁻⁹ M or higher, about 630×10⁻⁹ M or higher, about 640×10⁻⁹ M or higher, about 650×10⁻⁹ M or higher, about 660×10⁻⁹ M or higher, about 670×10⁻⁹ M or higher, about 680×10⁻⁹ M or higher, about 690×10⁻⁹ M or higher, about 700×10⁻⁹ M or higher, about 710×10⁻⁹ M or higher, about 720×10⁻⁹ M or higher, about 730×10⁻⁹ M or higher, about 740×10⁻⁹ M or higher, about 750×10⁻⁹ M or higher, about 760×10⁻⁹ M or higher, about 770×10⁻⁹ M or higher, about 780×10⁻⁹ M or higher, about 790×10⁻⁹ M or higher, about 800×10⁻⁹ M or higher, about 810×10⁻⁹ M or higher, about 820×10⁻⁹ M or higher, about 830×10⁻⁹ M or higher, about 840×10⁻⁹ M or higher, about 850×10⁻⁹ M or higher, about 860×10⁻⁹ M or higher, about 870×10⁻⁹ M or higher, about 880×10⁻⁹ M or higher, about 890×10⁻⁹ M or higher, about 900×10⁻⁹ M or higher, about 910×10⁻⁹ M or higher, about 920×10⁻⁹ M or higher, about 930×10⁻⁹ M or higher, about 940×10⁻⁹ M or higher, about 950×10⁻⁹ M or higher, about 960×10⁻⁹ M or higher, about 970×10⁻⁹ M or higher, about 980×10⁻⁹ M or higher, about 990×10⁻⁹ M or higher or about 1,000×10⁻⁹ M or higher.

In some embodiments, the first antigen binding domain specifically binds CD8 with the K_{D} of from about 0.1×10⁻⁹ M to about 1,000×10⁻⁹ M. In some embodiments, the first antigen binding domain specifically binds CD8 with the K_{D} of from about 0.5×10⁻⁹ M to about 700×10⁻⁹ M. In some embodiments, the first antigen binding domain specifically binds CD8 with the K_{D} of from about 0.5×10⁻⁹ M to about 500×10⁻⁹ M. In some embodiments, the first antigen binding domain specifically binds CD8 with the K_{D} of from about 0.5×10⁻⁹ M to about 400×10⁻⁹ M. In some embodiments, the first antigen binding domain specifically binds CD8 with the K_{D} of from about 1×10⁻⁹ M to about 400×10⁻⁹ M. In some embodiments, the first antigen binding domain specifically binds CD8 with the K_{D} of from about 0.5×10⁻⁹ M to about 300×10⁻⁹ M. In some embodiments, the first antigen binding domain specifically binds CD8 with the K_{D} of from about 1×10⁻⁹ M to about 300×10⁻⁹ M.

In some embodiments, the first antigen binding domain specifically binds CD8 with the K_{D} of about 0.1×10⁻⁹ M, such as about 0.2×10⁻⁹ M, about 0.3×10⁻⁹ M, about 0.4×10⁻⁹ M, about 0.5×10⁻⁹ M, about 0.6×10⁻⁹ M, about 0.7×10⁻⁹ M, about 0.8×10⁻⁹ M, about 0.9×10⁻⁹ M, about 50×10⁻⁹ M, about 55×10⁻⁹ M, about 60×10⁻⁹ M, about 65×10⁻⁹ M, about 70×10⁻⁹ M, about 75×10⁻⁹ M, about 80×10⁻⁹ M, about 85×10⁻⁹ M, about 90×10⁻⁹ M, about 95×10⁻⁹ M, about 100×10⁻⁹ M, about 110×10⁻⁹ M, about 120×10⁻⁹ M, about 130×10⁻⁹ M, about 140×10⁻⁹ M, about 150×10⁻⁹ M, about 160×10⁻⁹ M, about 170×10⁻⁹ M, about 180×10⁻⁹ M, about 190×10⁻⁹ M, about 200×10⁻⁹ M, about 210×10⁻⁹ M, about 220×10⁻⁹ M, about 230×10⁻⁹ M, about 240×10⁻⁹ M, about 250×10⁻⁹ M, about 260×10⁻⁹ M, about 270×10⁻⁹ M, about 280×10⁻⁹ M, about 290×10⁻⁹ M, about 300×10⁻⁹ M, about 310×10⁻⁹ M, about 320×10⁻⁹ M, about 330×10⁻⁹ M, about 340×10⁻⁹ M, about 350×10⁻⁹ M, about 360×10⁻⁹ M, about 370×10⁻⁹ M, about 380×10⁻⁹ M, about 390×10⁻⁹ M, about 400×10⁻⁹ M, about 410×10⁻⁹ M, about 420×10⁻⁹ M, about 430×10⁻⁹ M, about 440×10⁻⁹ M, about 450×10⁻⁹ M, about 460×10⁻⁹ M, about 470×10⁻⁹ M, about 480×10⁻⁹ M, about 490×10⁻⁹ M, about 400×10⁻⁹ M, about 510×10⁻⁹ M, about 520×10⁻⁹ M, about 530×10⁻⁹ M, about 540×10⁻⁹ M, about 550×10⁻⁹ M, about 560×10⁻⁹ M, about 570×10⁻⁹ M, about 580×10⁻⁹ M, about 590×10⁻⁹ M, about 600×10⁻⁹ M, about 610×10⁻⁹ M, about 620×10⁻⁹ M, about 630×10⁻⁹ M, about 640×10⁻⁹ M, about 650×10⁻⁹ M, about 660×10⁻⁹ M, about 670×10⁻⁹ M, about 680×10⁻⁹ M, about 690×10⁻⁹ M, about 700×10⁻⁹ M, about 710×10⁻⁹ M, about 720×10⁻⁹ M, about 730×10⁻⁹ M, about 740×10⁻⁹ M, about 750×10⁻⁹ M, about 760×10⁻⁹ M, about 770×10⁻⁹ M, about 780×10⁻⁹ M, about 790×10⁻⁹ M, about 800×10⁻⁹ M, about 810×10⁻⁹ M, about 820×10⁻⁹ M, about 830×10⁻⁹ M, about 840×10⁻⁹ M, about 850×10⁻⁹ M, about 860×10⁻⁹ M, about 870×10⁻⁹ M, about 880×10⁻⁹ M, about 890×10⁻⁹ M, about 900×10⁻⁹ M, about 910×10⁻⁹ M, about 920×10⁻⁹ M, about 930×10⁻⁹ M, about 940×10⁻⁹ M, about 950×10⁻⁹ M, about 960×10⁻⁹ M, about 970×10⁻⁹ M, about 980×10⁻⁹ M, about 990×10⁻⁹ M, or about 1,000×10⁻⁹ M.

In some embodiments, the second antigen binding domain specifically binds the TCR complex with the K_{D} of about 10×10⁻⁹ M or higher, such as about 20×10⁻⁹ M or higher, about 30×10⁻⁹ M or higher, about 40×10⁻⁹ M or higher, about 50×10⁻⁹ M or higher, such as about 55×10⁻⁹ M or higher, about 60×10⁻⁹ M or higher, about 65×10⁻⁹ M or higher, about 70×10⁻⁹ M or higher, about 75×10⁻⁹ M or higher, about 80×10⁻⁹ M or higher, about 85×10⁻⁹ M or higher, about 90×10⁻⁹ M or higher, about 95×10⁻⁹ M or higher, about 100×10⁻⁹ M or higher, about 110×10⁻⁹ M or higher, about 120×10⁻⁹ M or higher, about 130×10⁻⁹ M or higher, about 140×10⁻⁹ M or higher, about 150×10⁻⁹ M or higher, about 160×10⁻⁹ M or higher, about 170×10⁻⁹ M or higher, about 180×10⁻⁹ M or higher, about 190×10⁻⁹ M or higher, about 200×10⁻⁹ M or higher, about 210×10⁻⁹ M or higher, about 220×10⁻⁹ M or higher, about 230×10⁻⁹ M or higher, about 240×10⁻⁹ M or higher, about 250×10⁻⁹ M or higher, about 260×10⁻⁹ M or higher, about 270×10⁻⁹ M or higher, about 280×10⁻⁹ M or higher, about 290×10⁻⁹ M or higher, about 300×10⁻⁹ M or higher, about 310×10⁻⁹ M or higher, about 320×10⁻⁹ M or higher, about 330×10⁻⁹ M or higher, about 340×10⁻⁹ M or higher, about 350×10⁻⁹ M or higher, about 360×10⁻⁹ M or higher, about 370×10⁻⁹ M or higher, about 380×10⁻⁹ M or higher, about 390×10⁻⁹ M or higher, about 400×10⁻⁹ M or higher, about 410×10⁻⁹ M or higher, about 420×10⁻⁹ M or higher, about 430×10⁻⁹ M or higher, about 440×10⁻⁹ M or higher, about 450×10⁻⁹ M or higher, about 460×10⁻⁹ M or higher, about 470×10⁻⁹ M or higher, about 480×10⁻⁹ M or higher, about 490×10⁻⁹ M or higher, about 400×10⁻⁹ M or higher, about 510×10⁻⁹ M or higher, about 520×10⁻⁹ M or higher, about 530×10⁻⁹ M or higher, about 540×10⁻⁹ M or higher, about 550×10⁻⁹ M or higher, about 560×10⁻⁹ M or higher, about 570×10⁻⁹ M or higher, about 580×10⁻⁹ M or higher, about 590×10⁻⁹ M or higher, about 600×10⁻⁹ M or higher, about 610×10⁻⁹ M or higher, about 620×10⁻⁹ M or higher, about 630×10⁻⁹ M or higher, about 640×10⁻⁹ M or higher, about 650×10⁻⁹ M or higher, about 660×10⁻⁹ M or higher, about 670×10⁻⁹ M or higher, about 680×10⁻⁹ M or higher, about 690×10⁻⁹ M or higher, about 700×10⁻⁹ M or higher, about 710×10⁻⁹ M or higher, about 720×10⁻⁹ M or higher, about 730×10⁻⁹ M or higher, about 740×10⁻⁹ M or higher, about 750×10⁻⁹ M or higher, about 760×10⁻⁹ M or higher, about 770×10⁻⁹ M or higher, about 780×10⁻⁹ M or higher, about 790×10⁻⁹ M or higher, about 800×10⁻⁹ M or higher, about 810×10⁻⁹ M or higher, about 820×10⁻⁹ M or higher, about 830×10⁻⁹ M or higher, about 840×10⁻⁹ M or higher, about 850×10⁻⁹ M or higher, about 860×10⁻⁹ M or higher, about 870×10⁻⁹ M or higher, about 880×10⁻⁹ M or higher, about 890×10⁻⁹ M or higher, about 900×10⁻⁹ M or higher, about 910×10⁻⁹ M or higher, about 920×10⁻⁹ M or higher, about 930×10⁻⁹ M or higher, about 940×10⁻⁹ M or higher, about 950×10⁻⁹ M or higher, about 960×10⁻⁹ M or higher, about 970×10⁻⁹ M or higher, about 980×10⁻⁹ M or higher, about 990×10⁻⁹ M or higher or about 1,000×10⁻⁹ M or higher.

In some embodiments, the second antigen binding domain specifically binds the TCR complex with the K_{D} of from about 50×10⁻⁹ M to about 1,000×10⁻⁹ M. In some embodiments, the second antigen binding domain specifically binds the TCR complex with the K_{D} of from about 50×10⁻⁹ M to about 700×10⁻⁹ M. In some embodiments, the second antigen binding domain specifically binds the TCR complex with the K_{D} of from about 50×10⁻⁹ M to about 500×10⁻⁹ M. In some embodiments, the second antigen binding domain specifically binds the TCR complex with the K_{D} of from about 50×10⁻⁹ M to about 400×10⁻⁹ M. In some embodiments, the second antigen binding domain specifically binds the TCR complex with the K_{D} of from about 100×10⁻⁹ M to about 400×10⁻⁹ M. In some embodiments, the second antigen binding domain specifically binds the TCR complex with the K_{D} of from about 50×10⁻⁹ M to about 300×10⁻⁹ M. In some embodiments, the second antigen binding domain specifically binds the TCR complex with the K_{D} of from about 100×10⁻⁹ M to about 300×10⁻⁹ M.

In some embodiments, the second antigen binding domain specifically binds the TCR complex with the K_{D} of about 50×10⁻⁹ M, about 55×10⁻⁹ M, about 60×10⁻⁹ M, about 65×10⁻⁹ M, about 70×10⁻⁹ M, about 75×10⁻⁹ M, about 80×10⁻⁹ M, about 85×10⁻⁹ M, about 90×10⁻⁹ M, about 95×10⁻⁹ M, about 100×10⁻⁹ M, about 110×10⁻⁹ M, about 120×10⁻⁹ M, about 130×10⁻⁹ M, about 140×10⁻⁹ M, about 150×10⁻⁹ M, about 160×10⁻⁹ M, about 170×10⁻⁹ M, about 180×10⁻⁹ M, about 190×10⁻⁹ M, about 200×10⁻⁹ M, about 210×10⁻⁹ M, about 220×10⁻⁹ M, about 230×10⁻⁹ M, about 240×10⁻⁹ M, about 250×10⁻⁹ M, about 260×10⁻⁹ M, about 270×10⁻⁹ M, about 280×10⁻⁹ M, about 290×10⁻⁹ M, about 300×10⁻⁹ M, about 310×10⁻⁹ M, about 320×10⁻⁹ M, about 330×10⁻⁹ M, about 340×10⁻⁹ M, about 350×10⁻⁹ M, about 360×10⁻⁹ M, about 370×10⁻⁹ M, about 380×10⁻⁹ M, about 390×10⁻⁹ M, about 400×10⁻⁹ M, about 410×10⁻⁹ M, about 420×10⁻⁹ M, about 430×10⁻⁹ M, about 440×10⁻⁹ M, about 450×10⁻⁹ M, about 460×10⁻⁹ M, about 470×10⁻⁹ M, about 480×10⁻⁹ M, about 490×10⁻⁹ M, about 400×10⁻⁹ M, about 510×10⁻⁹ M, about 520×10⁻⁹ M, about 530×10⁻⁹ M, about 540×10⁻⁹ M, about 550×10⁻⁹ M, about 560×10⁻⁹ M, about 570×10⁻⁹ M, about 580×10⁻⁹ M, about 590×10⁻⁹ M, about 600×10⁻⁹ M, about 610×10⁻⁹ M, about 620×10⁻⁹ M, about 630×10⁻⁹ M, about 640×10⁻⁹ M, about 650×10⁻⁹ M, about 660×10⁻⁹ M, about 670×10⁻⁹ M, about 680×10⁻⁹ M, about 690×10⁻⁹ M, about 700×10⁻⁹ M, about 710×10⁻⁹ M, about 720×10⁻⁹ M, about 730×10⁻⁹ M, about 740×10⁻⁹ M, about 750×10⁻⁹ M, about 760×10⁻⁹ M, about 770×10⁻⁹ M, about 780×10⁻⁹ M, about 790×10⁻⁹ M, about 800×10⁻⁹ M, about 810×10⁻⁹ M, about 820×10⁻⁹ M, about 830×10⁻⁹ M, about 840×10⁻⁹ M, about 850×10⁻⁹ M, about 860×10⁻⁹ M, about 870×10⁻⁹ M, about 880×10⁻⁹ M, about 890×10⁻⁹ M, about 900×10⁻⁹ M, about 910×10⁻⁹ M, about 920×10⁻⁹ M, about 930×10⁻⁹ M, about 940×10⁻⁹ M, about 950×10⁻⁹ M, about 960×10⁻⁹ M, about 970×10⁻⁹ M, about 980×10⁻⁹ M, about 990×10⁻⁹ M, or about 1,000×10⁻⁹ M.

In some embodiments, the third antigen binding domain specifically binds the antigen expressed by the undesired cell with the K_{D} of about 5×10⁻⁸ M or less, such as about 1×10⁻⁸ M or less, about 5×10⁻⁹ M or less, about 1×10⁻⁹ M or less, about 5×10⁻¹° M or less, about 1×10⁻¹° M or less, about 5×10⁻¹¹ M or less, about 1×10⁻¹¹ M or less, about 5×10⁻¹² M or less, about 1×10⁻¹² M or less, about 5×10⁻¹³ M or less, about 1×10⁻¹³ M or less, about 5×10⁻¹⁴ M or less, about 1×10⁻¹⁴ M or less, about 5×10⁻¹⁵ M or less or about 1×10⁻¹⁵ M or less.

In some embodiments, the third antigen binding domain specifically binds the antigen expressed by the undesired cell with the K_{D} of from about 5×10⁻⁸ M to about 1×10⁻¹⁵ M. In some embodiments, the third antigen binding domain specifically binds the antigen expressed by the undesired cell with the K_{D} of from about 1×10⁻⁹ M to about 1×10¹⁵ M. In some embodiments, the third antigen binding domain specifically binds the antigen expressed by the undesired cell with the K_{D} of from about 5×10⁻¹° M to about 1×10⁻¹⁵ M. In some embodiments, the third antigen binding domain specifically binds the antigen expressed by the undesired cell with the K_{D} of from about 1×10⁻¹° M to about 1×10¹⁵ M. In some embodiments, the third antigen binding domain specifically binds the antigen expressed by the undesired cell with the K_{D} of from about 5×10⁻¹¹ M to about 1×10¹⁵ M. In some embodiments, the third antigen binding domain specifically binds the antigen expressed by the undesired cell with the K_{D} of from about 1×10⁻¹¹ M to about 1×10⁻¹⁵ M.

In some embodiments, the third antigen binding domain specifically binds the antigen expressed by the undesired cell with the K_{D} of about 5×10⁻⁸ M, such as about 1×10⁻⁸ M, about 5×10⁻⁹ M, about 1×10⁻⁹ M, about 5×10⁻¹° M, about 1×10⁻¹° M, about 5×10⁻¹¹ M, about 1×10⁻¹¹ M, about 5×10⁻¹² M, about 1×10⁻¹² M, about 5×10⁻¹³ M, about 1×10⁻¹³ M, about 5×10⁻¹⁴ M, about 1×10⁻¹⁴ M, about 5×10⁻¹⁵ M, or about 1×10⁻¹⁵ M.

In some embodiments, the first antigen binding domain specifically binds CD8 with the K_{D} of from about 0.1×10⁻⁹ M to about 1,000×10⁻⁹ M and the second antigen binding domain specifically binds the TCR complex with the K_{D} of from about 50×10⁻⁹ M to about 1,000×10⁻⁹ M. In some embodiments, the first antigen binding domain specifically binds CD8 with the K_{D} of from about 0.5×10⁻⁹ M to about 500×10⁻⁹ M and the second antigen binding domain specifically binds the TCR complex with the K_{D} of from about 50×10⁻⁹ M to about 500×10⁻⁹ M. In some embodiments, the first antigen binding domain specifically binds CD8 with the K_{D} of from about 1×10⁻⁹ M to about 500×10⁻⁹ M and the second antigen binding domain specifically binds the TCR complex with the K_{D} of from about 100×10⁻⁹ M to about 500×10⁻⁹ M.

In some embodiments, the first antigen binding domain specifically binds CD8 with the K_{D} about 0.5×10⁻⁹ M or higher and the second antigen binding domain specifically binds the TCR complex with the K_{D} of about 50×10⁻⁹ M or higher. In some embodiments, the first antigen binding domain specifically binds CD8 with the K_{D} about 1×10⁻⁹ M or higher and the second antigen binding domain specifically binds the TCR complex with the K_{D} of about 100×10⁻⁹ M or higher.

In some embodiments, the first antigen binding domain specifically binds CD8 with the K_{D} of from about 0.1×10⁻⁹ M to about 1,000×10⁻⁹ M, the second antigen binding domain specifically binds the TCR complex with the K_{D} of from about 50×10⁻⁹ M to about 1,000×10⁻⁹ M, and the third antigen binding domain specifically binds the antigen expressed by the undesired cell with the K_{D} of from about 5×10⁻⁸ M to about 1×10⁻¹⁵ M. In some embodiments, the first antigen binding domain specifically binds CD8 with the K_{D} of from about 0.5×10⁻⁹ M to about 500×10⁻⁹ M, the second antigen binding domain specifically binds the TCR complex with the K_{D} of from about 50×10⁻⁹ M to about 500×10⁻⁹ M, and the third antigen binding domain specifically binds the antigen expressed by the undesired cell with the K_{D} of from about 1×10⁻⁹ M to about 1×10¹⁵ M. In some embodiments, the first antigen binding domain specifically binds CD8 with the K_{D} of from about 1×10⁻⁹ M to about 500×10⁻⁹ M, the second antigen binding domain specifically binds the TCR complex with the K_{D} of from about 100×10⁻⁹ M to about 500×10⁻⁹ M, and the third antigen binding domain specifically binds the antigen expressed by the undesired cell with the K_{D} of from about 1×10¹⁰ M to about 1×10⁻¹⁵ M.

In some embodiments, the first antigen binding domain specifically binds CD8 with the K_{D} about 0.5×10⁻⁹ M or higher, the second antigen binding domain specifically binds the TCR complex with the K_{D} of about 50×10⁻⁹ M or higher, and the third antigen binding domain specifically binds the antigen expressed by the undesired cell with the K_{D} of about 1×10⁻⁸ M or less. In some embodiments, the first antigen binding domain specifically binds CD8 with the K_{D} about 1×10⁻⁹ M or higher, the second antigen binding domain specifically binds the TCR complex with the K_{D} of about 100×10⁻⁹ M or higher, and the third antigen binding domain specifically binds the antigen expressed by the undesired cell with the K_{D} of about 1×10⁻⁹ M or less.

In some embodiments, the first antigen binding domain comprises a scFv, a Fab, a Fab′, a F(ab′)₂, a Fd, a Fv, a domain antibody (dAb), a VHH domain, a VH, a VL, a non-antibody scaffold, or fragments thereof. In some embodiments, the second antigen binding domain comprises a scFv, a Fab, a Fab′, a F(ab′)₂, a Fd, a Fv, a dAb, a VHH domain, a VH, a VL, a non-antibody scaffold, or fragments thereof. In some embodiments, the third antigen binding domain comprises a scFv, a Fab, a Fab′, a F(ab′)₂, a Fd, a Fv, a dAb, a VHH domain, a VH, a VL, a non-antibody scaffold, or fragments thereof.

In some embodiments, the first antigen binding domain comprises a scFv. In some embodiments, the first antigen binding domain comprises a Fab. In some embodiments, the first antigen binding domain comprises a Fab′. In some embodiments, the first antigen binding domain comprises a F(ab′)₂. In some embodiments, the first antigen binding domain comprises a Fd. In some embodiments, the first antigen binding domain comprises a Fv. In some embodiments, the first antigen binding domain comprises a dAb. In some embodiments, the first antigen binding domain comprises a VHH. In some embodiments, the first antigen binding domain comprises a VH. In some embodiments, the first antigen binding domain comprises a VL. In some embodiments, the first antigen binding domain comprises a non-antibody scaffold. In some embodiments, the second antigen binding domain comprises a scFv. In some embodiments, the second antigen binding domain comprises a Fab. In some embodiments, the second antigen binding domain comprises a Fab′. In some embodiments, the second antigen binding domain comprises a F(ab′)₂. In some embodiments, the second antigen binding domain comprises a Fd. In some embodiments, the second antigen binding domain comprises a Fv. In some embodiments, the second antigen binding domain comprises a dAb. In some embodiments, the second antigen binding domain comprises a VHH. In some embodiments, the second antigen binding domain comprises a VH. In some embodiments, the second antigen binding domain comprises a VL. In some embodiments, the second antigen binding domain comprises a non-antibody scaffold. In some embodiments, the third antigen binding domain comprises a scFv. In some embodiments, the third antigen binding domain comprises a Fab. In some embodiments, the third antigen binding domain comprises a Fab′. In some embodiments, the third antigen binding domain comprises a F(ab′)₂. In some embodiments, the third antigen binding domain comprises a Fd. In some embodiments, the third antigen binding domain comprises a Fv. In some embodiments, the third antigen binding domain comprises a dAb. In some embodiments, the third antigen binding domain comprises a VHH. In some embodiments, the third antigen binding domain comprises a VH. In some embodiments, the third antigen binding domain comprises a VL. In some embodiments, the third antigen binding domain comprises a non-antibody scaffold. In some embodiments, the first antigen binding domain comprises a scFv, the second antigen binding domain comprises a scFv and the third antigen binding domain comprises a Fab.

The disclosure also provides an isolated molecule, comprising: a first polypeptide, a second polypeptide and a third polypeptide, wherein the first polypeptide comprises, from N- to C-terminus, a second antigen binding domain comprising a scFv that specifically binds a TCR complex, a VH that is capable of specifically binding CD8, a CH1 domain, a hinge, a CH2 domain and a CH3 domain; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc.

The disclosure also provides an isolated molecule, comprising a first polypeptide, a second polypeptide and a third polypeptide, wherein the first polypeptide comprises, from N- to C-terminus, a VH that is capable of specifically binding CD8, a CH1 domain, a hinge, a CH2 domain and a CH3 domain; the second polypeptide comprises, from N- to C-terminus, a VL that is capable of specifically binding CD8, a CL domain and a second antigen binding domain comprising a scFv that specifically binds a TCR complex; and the third polypeptide comprises, from N- to C-terminus, a third antigen binding domain comprising a scFv that specifically binds an antigen expressed by an undesired cell and a Fc or a fragment of the Fc.

The disclosure also provides an isolated molecule, comprising: a first polypeptide, a second polypeptide and a third polypeptide, wherein the first polypeptide comprises, from N- to C-terminus, a VH that is capable of specifically CD8, a CH1 domain, a hinge, a CH2 domain, a CH3 domain and a second antigen binding domain comprising a scFv that specifically binds a TCR complex; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc.

“Capable of specifically binding” in the context of CD8 refers to VH and VL which specifically bind CD8 when they associate to form an antigen binding domain. The VH that is capable of specifically binding CD8 may specifically bind CD8 in the absence of the VL in instances when most paratope residues reside in the VH.

In some embodiments, first antigen binding domain comprising the Fab, the second antigen binding domain comprising the scFv or the third antigen binding domain comprising the scFv is conjugated to the Fc or the fragment of the Fc, to the VH that is capable of specifically biding CD8, to the CL domain or to the CH3 domain via a linker.

In some embodiments, the linker comprises a polypeptide having an amino acid sequence of any one of SEQ ID NOs: 2183-2290.

In some embodiments, the fragment of the Fc comprises a CH2 domain and a CH3 domain.

In some embodiments, the CH3 domain comprises one or more substitutions when compared to a wild-type CH3 domain. An exemplary wild-type CH3 domain is an IgG1 CH3 domain having the amino acid sequence of SEQ ID NO: 2319.

In some embodiments, the one or more substitutions comprise T350V, L351Y, F405A, Y407V, T366Y, T366W, F405W, T394W, T394S, Y407T, Y407A, T366S/L368A/Y407V, L351Y/F405A/Y407V, T366A/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V or T350V/T366L/K392L/T394W, wherein residue numbering is according to the EU index.

In some embodiments, the Fc, the CH2 domain or the CH3 domain is an IgG1 isotype. In some embodiments, the Fc, the CH2 domain or the CH3 domain is an IgG2 isotype. In some embodiments, the Fc, the CH2 domain or the CH3 domain is an IgG3 isotype. In some embodiments, the Fc, the CH2 domain or the CH3 domain is an IgG4 isotype.

In some embodiments, the second antigen binding domain specifically binds CD3, TCRα chain, TCRβ chain, TCRγ chain or TCRδ chain, or any combination thereof. In some embodiments, the second antigen binding domain specifically binds CD3. In some embodiments, the second antigen binding domain specifically binds CD3ε. In some embodiments, the second antigen binding domain specifically binds TCRα chain. In some embodiments, the second antigen binding domain specifically binds TCRβ chain. In some embodiments, the second antigen binding domain specifically binds TCRγ chain. In some embodiments, the second antigen binding domain specifically binds TCRδ chain.

In some embodiments, the TCRβ chain comprises TCRVB17.

In some embodiments, CD3 comprises CD3ε, CD3γ, CD3δ or CD3ζ. In some embodiments, CD3 comprises CD3ε. In some embodiments, CD3 comprises CD3γ. In some embodiments, CD3 comprises CD3δ. In some embodiments, CD3 comprises CD3ζ.

In some embodiments, the TCR complex and the CD8 are from a mammal. In some embodiments, the TCR complex and the CD8 are from a rodent. In some embodiments, the TCR complex and the CD8 are from a human. In some embodiments, the TCR complex and the CD8 are from a monkey. In some embodiments, the TCR complex and the CD8 are from a dog. In some embodiments, the TCR complex and the CD8 are from a rat. In some embodiments, the TCR complex and the CD8 are from a mouse.

In some embodiments, the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

In some embodiments, the first antigen binding domain that specifically binds CD8 comprises the VH of SEQ ID NO: 2313 and the VL of SEQ ID NO: 2314.

In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:32. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:133; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:168. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:201; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:202. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:269; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:304. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:337; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:338. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:372. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:440. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:542. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:576. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:609; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:610. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:644. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:678. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:712. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:746. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:780. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:814. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:847; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:848. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:881; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:882. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:915; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:916. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:949; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:950. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:983; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:984. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1017; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1018. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1051; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1052. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1085; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1086. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1119; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1120. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1153; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1154. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1187; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1188. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1221; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1222. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1255; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1256. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1289; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1290. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1323; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1324. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1357; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1358. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1391; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1392. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1425; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1426. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1459; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1460. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1493; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1494. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1527; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1528. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1561; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1562. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1595; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1596. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1629; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1630. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1663; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1664. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1697; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1698. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1731; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1732. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1765; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1766. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1799; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1800. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1833; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1834. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1867; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1868. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1901; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1902. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1935; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1936. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1969; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1970. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2003; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2004. In another aspect, provided herein is an antibody that binds CD8. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2037; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2038. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2071; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2072. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2105; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2106. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2139; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2140. In some embodiments, the first antigen binding domain that specifically binds CD8 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2173; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2174. In some embodiments, the VH CDR1, VH CDR2, VH CDR3, VL CDR1, VL CDR2, and VL CDR3 amino acid sequences of the first antigen binding domain that specifically binds CD8 are according to the Kabat numbering system. In some embodiments, the VH CDR1, VH CDR2, VH CDR3, VL CDR1, VL CDR2, and VL CDR3 amino acid sequences of the first antigen binding domain that specifically binds CD8 are according to the Chothia numbering system. In some embodiments, the VH CDR1, VH CDR2, VH CDR3, VL CDR1, VL CDR2, and VL CDR3 amino acid sequences of the first antigen binding domain that specifically binds CD8 are according to the AbM numbering system. In some embodiments, the VH CDR1, VH CDR2, VH CDR3, VL CDR1, VL CDR2, and VL CDR3 amino acid sequences of the first antigen binding domain that specifically binds CD8 are according to the Contact numbering system. In some embodiments, the VH CDR1, VH CDR2, VH CDR3, VL CDR1, VL CDR2, and VL CDR3 amino acid sequences of the first antigen binding domain that specifically binds CD8 are according to the IMGT numbering system.

In some embodiments, the first antigen binding domain that specifically binds CD8 binds a CD8 antigen. In some embodiments, the first antigen binding domain that specifically binds CD8 binds a CD8 epitope. In some embodiments, the VH CDR1, VH CDR2, VH CDR3, VL CDR1, VL CDR2 and VL CDR3 form a binding site for an antigen of the CD8. In some embodiments, the VH CDR1, VH CDR2, VH CDR3, VL CDR1, VL CDR2 and VL CDR3 form a binding site for an epitope of the CD8. In some embodiments, the CD8 is present on the surface of a T cell.

In some embodiments, the first antigen binding domain that specifically binds CD8 binds to CD8α. In some embodiments, the first antigen binding domain that specifically binds CD8 binds a CD8α antigen. In some embodiments, the first antigen binding domain that specifically binds CD8 binds a CD8α epitope. In some embodiments, the VH CDR1, VH CDR2, VH CDR3, VL CDR1, VL CDR2 and VL CDR3 form a binding site for an antigen of the CD8α. In some embodiments, the VH CDR1, VH CDR2, VH CDR3, VL CDR1, VL CDR2 and VL CDR3 form a binding site for an epitope of the CD8α. In some embodiments, the CD8α is present on the surface of a T cell.

In some embodiments, the first antigen binding domain that specifically binds CD8 binds to CD8β. In some embodiments, the first antigen binding domain that specifically binds CD8 binds a CD8β antigen. In some embodiments, the first antigen binding domain that specifically binds CD8 binds a CD8β epitope. In some embodiments, the VH CDR1, VH CDR2, VH CDR3, VL CDR1, VL CDR2 and VL CDR3 form a binding site for an antigen of the CD8β. In some embodiments, the VH CDR1, VH CDR2, VH CDR3, VL CDR1, VL CDR2 and VL CDR3 form a binding site for an epitope of the CD8β. In some embodiments, the CD8β is present on the surface of a T cell.

In some embodiments, the first antigen binding domain that specifically binds CD8 binds at the interface of CD8α and CD8β. In some embodiments, the first antigen binding domain that specifically binds CD8 binds an antigen at the interface of CD8α and CD8β. In some embodiments, the first antigen binding domain that specifically binds CD8 binds an epitope at the interface of CD8α and CD8β. In some embodiments, the VH CDR1, VH CDR2, VH CDR3, VL CDR1, VL CDR2 and VL CDR3 form a binding site for an antigen at the interface of CD8α and CD8β. In some embodiments, the VH CDR1, VH CDR2, VH CDR3, VL CDR1, VL CDR2 and VL CDR3 form a binding site for an epitope at the interface of CD8α and CD8β. In some embodiments, the interface of CD8α and CD8β is present on the surface of a T cell.

In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system. Exemplary sets of 6 CDRs (VH CDR1-3 and VL CDR1-3) of certain antibody embodiments are provided herein. Other sets of CDRs are contemplated and within the scope of the antibody embodiments provided herein.

In one embodiment, the first antigen binding domain that specifically binds CD8, comprises: (i) a VH comprising a VH CDR1, a VH CDR2, and a VH CDR3 having an amino acid sequence of SEQ ID NOs:1, 2, and 3, respectively, and (ii) a VL comprising a VL CDR1, VL CDR2, and VL CDR3 having an amino acid sequence of SEQ ID NOs:4, 5, and 6, respectively. In one embodiment, the first antigen binding domain that specifically binds CD8, comprises: (i) a VH comprising a VH CDR1, a VH CDR2, and a VH CDR3 having an amino acid sequence of SEQ ID NOs:7, 8, and 9, respectively, and (ii) a VL comprising a VL CDR1, VL CDR2, and VL CDR3 having an amino acid sequence of SEQ ID NOs:10, 11, and 12, respectively. In one embodiment, the first antigen binding domain that specifically binds CD8, comprises: (i) a VH comprising a VH CDR1, a VH CDR2, and a VH CDR3 having an amino acid sequence of SEQ ID NOs:13, 14, and 15, respectively, and (ii) a VL comprising a VL CDR1, VL CDR2, and VL CDR3 having an amino acid sequence of SEQ ID NOs:16, 17, and 18, respectively. In one embodiment, the first antigen binding domain that specifically binds CD8, comprises: (i) a VH comprising a VH CDR1, a VH CDR2, and a VH CDR3 having an amino acid sequence of SEQ ID NOs:19, 20, and 21, respectively, and (ii) a VL comprising a VL CDR1, VL CDR2, and VL CDR3 having an amino acid sequence of SEQ ID NOs:22, 23, and 24, respectively. In one embodiment, the first antigen binding domain that specifically binds CD8, comprises: (i) a VH comprising a VH CDR1, a VH CDR2, and a VH CDR3 having an amino acid sequence of SEQ ID NOs:25, 26, and 27, respectively, and (ii) a VL comprising a VL CDR1, VL CDR2, and VL CDR3 having an amino acid sequence of SEQ ID NOs:28, 29, and 30, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:31; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:32. In one aspect, provided herein is an antibody that binds CD8, comprising a VH having an amino acid sequence of SEQ ID NO:31. In one aspect, provided herein is an antibody that binds CD8, comprising a VL having an amino acid sequence of SEQ ID NO:32. In one aspect, provided herein is an antibody that binds CD8, comprising a VH having an amino acid sequence of SEQ ID NO:31, and a VL having an amino acid sequence of SEQ ID NO:32. In one aspect, provided herein is an antibody that binds CD8, comprising a heavy chain having an amino acid sequence of SEQ ID NO:33. In one aspect, provided herein is an antibody that binds CD8, comprising a light chain having an amino acid sequence of SEQ ID NO:34. In one aspect, provided herein is an antibody that binds CD8, comprising a heavy chain having an amino acid sequence of SEQ ID NO:33, and a light chain having an amino acid sequence of SEQ ID NO:34. In one aspect, provided herein is an antibody that binds CD8, comprising a VH having an amino acid sequence having at least 95% identity to an amino acid sequence of SEQ ID NO:31. In one aspect, provided herein is an antibody that binds CD8, comprising a VL having an amino acid sequence having at least 95% identity to an amino acid sequence of SEQ ID NO:32. In one aspect, provided herein is an antibody that binds CD8, comprising a VH having an amino acid sequence having at least 95% identity to an amino acid sequence of SEQ ID NO:31, and a VL having an amino acid sequence having at least 95% identity to an amino acid sequence of SEQ ID NO:32. In one aspect, provided herein is an antibody that binds CD8, comprising a heavy chain having an amino acid sequence having at least 95% identity to an amino acid sequence of SEQ ID NO:33. In one aspect, provided herein is an antibody that binds CD8, comprising a light chain having an amino acid sequence having at least 95% identity to an amino acid sequence of SEQ ID NO:34. In one aspect, provided herein is an antibody that binds CD8, comprising a heavy chain having an amino acid sequence having at least 95% identity to an amino acid sequence of SEQ ID NO:33, and a light chain having an amino acid sequence having at least 95% identity to an amino acid sequence of SEQ ID NO:34.

In one embodiment, the first antigen binding domain that specifically binds CD8, comprises: (i) a VH comprising a VH CDR1, a VH CDR2, and a VH CDR3 having an amino acid sequence of SEQ ID NOs:35, 36, and 37, respectively, and (ii) a VL comprising a VL CDR1, VL CDR2, and VL CDR3 having an amino acid sequence of SEQ ID NOs:38, 39, and 40, respectively. In one embodiment, the first antigen binding domain that specifically binds CD8, comprises: (i) a VH comprising a VH CDR1, a VH CDR2, and a VH CDR3 having an amino acid sequence of SEQ ID NOs:41, 42, and 43, respectively, and (ii) a VL comprising a VL CDR1, VL CDR2, and VL CDR3 having an amino acid sequence of SEQ ID NOs:44, 45, and 46, respectively. In one embodiment, the first antigen binding domain that specifically binds CD8, comprises: (i) a VH comprising a VH CDR1, a VH CDR2, and a VH CDR3 having an amino acid sequence of SEQ ID NOs:47, 48, and 49, respectively, and (ii) a VL comprising a VL CDR1, VL CDR2, and VL CDR3 having an amino acid sequence of SEQ ID NOs:50, 51, and 52, respectively. In one embodiment, the first antigen binding domain that specifically binds CD8, comprises: (i) a VH comprising a VH CDR1, a VH CDR2, and a VH CDR3 having an amino acid sequence of SEQ ID NOs:53, 54, and 55, respectively, and (ii) a VL comprising a VL CDR1, VL CDR2, and VL CDR3 having an amino acid sequence of SEQ ID NOs:56, 57, and 58, respectively. In one embodiment, the first antigen binding domain that specifically binds CD8, comprises: (i) a VH comprising a VH CDR1, a VH CDR2, and a VH CDR3 having an amino acid sequence of SEQ ID NOs:59, 60, and 61, respectively, and (ii) a VL comprising a VL CDR1, VL CDR2, and VL CDR3 having an amino acid sequence of SEQ ID NOs:62, 63, and 64, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:66. In one aspect, provided herein is an antibody that binds CD8, comprising a VH having an amino acid sequence of SEQ ID NO:65. In one aspect, provided herein is an antibody that binds CD8, comprising a VL having an amino acid sequence of SEQ ID NO:66. In one aspect, provided herein is an antibody that binds CD8, comprising a VH having an amino acid sequence of SEQ ID NO:65, and a VL having an amino acid sequence of SEQ ID NO:66. In one aspect, provided herein is an antibody that binds CD8, comprising a heavy chain having an amino acid sequence of SEQ ID NO:67. In one aspect, provided herein is an antibody that binds CD8, comprising a light chain having an amino acid sequence of SEQ ID NO:68. In one aspect, provided herein is an antibody that binds CD8, comprising a heavy chain having an amino acid sequence of SEQ ID NO:67, and a light chain having an amino acid sequence of SEQ ID NO:68. In one aspect, provided herein is an antibody that binds CD8, comprising a VH having an amino acid sequence having at least 95% identity to an amino acid sequence of SEQ ID NO:65. In one aspect, provided herein is an antibody that binds CD8, comprising a VL having an amino acid sequence having at least 95% identity to an amino acid sequence of SEQ ID NO:66. In one aspect, provided herein is an antibody that binds CD8, comprising a VH having an amino acid sequence having at least 95% identity to an amino acid sequence of SEQ ID NO:65, and a VL having an amino acid sequence having at least 95% identity to an amino acid sequence of SEQ ID NO:66. In one aspect, provided herein is an antibody that binds CD8, comprising a heavy chain having an amino acid sequence having at least 95% identity to an amino acid sequence of SEQ ID NO:67. In one aspect, provided herein is an antibody that binds CD8, comprising a light chain having an amino acid sequence having at least 95% identity to an amino acid sequence of SEQ ID NO:68. In one aspect, provided herein is an antibody that binds CD8, comprising a heavy chain having an amino acid sequence having at least 95% identity to an amino acid sequence of SEQ ID NO:67, and a light chain having an amino acid sequence having at least 95% identity to an amino acid sequence of SEQ ID NO:68.

In one embodiment, the first antigen binding domain that specifically binds CD8, comprises: (i) a VH comprising a VH CDR1, a VH CDR2, and a VH CDR3 having an amino acid sequence of SEQ ID NOs:69, 70, and 71, respectively, and (ii) a VL comprising a VL CDR1, VL CDR2, and VL CDR3 having an amino acid sequence of SEQ ID NOs:72, 73, and 74, respectively. In one embodiment, the first antigen binding domain that specifically binds CD8, comprises: (i) a VH comprising a VH CDR1, a VH CDR2, and a VH CDR3 having an amino acid sequence of SEQ ID NOs:75, 76, and 77, respectively, and (ii) a VL comprising a VL CDR1, VL CDR2, and VL CDR3 having an amino acid sequence of SEQ ID NOs:78, 79, and 80, respectively. In one embodiment, the first antigen binding domain that specifically binds CD8, comprises: (i) a VH comprising a VH CDR1, a VH CDR2, and a VH CDR3 having an amino acid sequence of SEQ ID NOs:81, 82, and 83, respectively, and (ii) a VL comprising a VL CDR1, VL CDR2, and VL CDR3 having an amino acid sequence of SEQ ID NOs:84, 85, and 86, respectively. In one embodiment, the first antigen binding domain that specifically binds CD8, comprises: (i) a VH comprising a VH CDR1, a VH CDR2, and a VH CDR3 having an amino acid sequence of SEQ ID NOs:87, 88, and 89, respectively, and (ii) a VL comprising a VL CDR1, VL CDR2, and VL CDR3 having an amino acid sequence of SEQ ID NOs:90, 91, and 92, respectively. In one embodiment, the first antigen binding domain that specifically binds CD8, comprises: (i) a VH comprising a VH CDR1, a VH CDR2, and a VH CDR3 having an amino acid sequence of SEQ ID NOs:93, 94, and 95, respectively, and (ii) a VL comprising a VL CDR1, VL CDR2, and VL CDR3 having an amino acid sequence of SEQ ID NOs:96, 97, and 98, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:99; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:100. In one aspect, provided herein is an antibody that binds CD8, comprising a VH having an amino acid sequence of SEQ ID NO:99. In one aspect, provided herein is an antibody that binds CD8, comprising a VL having an amino acid sequence of SEQ ID NO:100. In one aspect, provided herein is an antibody that binds CD8, comprising a VH having an amino acid sequence of SEQ ID NO:99, and a VL having an amino acid sequence of SEQ ID NO:100. In one aspect, provided herein is an antibody that binds CD8, comprising a heavy chain having an amino acid sequence of SEQ ID NO:101. In one aspect, provided herein is an antibody that binds CD8, comprising a light chain having an amino acid sequence of SEQ ID NO:102. In one aspect, provided herein is an antibody that binds CD8, comprising a heavy chain having an amino acid sequence of SEQ ID NO:101, and a light chain having an amino acid sequence of SEQ ID NO:102. In one aspect, provided herein is an antibody that binds CD8, comprising a VH having an amino acid sequence having at least 95% identity to an amino acid sequence of SEQ ID NO:99. In one aspect, provided herein is an antibody that binds CD8, comprising a VL having an amino acid sequence having at least 95% identity to an amino acid sequence of SEQ ID NO:100. In one aspect, provided herein is an antibody that binds CD8, comprising a VH having an amino acid sequence having at least 95% identity to an amino acid sequence of SEQ ID NO:99, and a VL having an amino acid sequence having at least 95% identity to an amino acid sequence of SEQ ID NO:100. In one aspect, provided herein is an antibody that binds CD8, comprising a heavy chain having an amino acid sequence having at least 95% identity to an amino acid sequence of SEQ ID NO:101. In one aspect, provided herein is an antibody that binds CD8, comprising a light chain having an amino acid sequence having at least 95% identity to an amino acid sequence of SEQ ID NO:102. In one aspect, provided herein is an antibody that binds CD8, comprising a heavy chain having an amino acid sequence having at least 95% identity to an amino acid sequence of SEQ ID NO:101, and a light chain having an amino acid sequence having at least 95% identity to an amino acid sequence of SEQ ID NO:102.

In one embodiment, the first antigen binding domain that specifically binds CD8, comprises: (i) a VH comprising a VH CDR1, a VH CDR2, and a VH CDR3 having an amino acid sequence of SEQ ID NOs:103, 104, and 105, respectively, and (ii) a VL comprising a VL CDR1, VL CDR2, and VL CDR3 having an amino acid sequence of SEQ ID NOs:106, 107, and 108, respectively. In one embodiment, the first antigen binding domain that specifically binds CD8, comprises: (i) a VH comprising a VH CDR1, a VH CDR2, and a VH CDR3 having an amino acid sequence of SEQ ID NOs:109, 110, and 111, respectively, and (ii) a VL comprising a VL CDR1, VL CDR2, and VL CDR3 having an amino acid sequence of SEQ ID NOs:112, 113, and 114, respectively. In one embodiment, the first antigen binding domain that specifically binds CD8, comprises: (i) a VH comprising a VH CDR1, a VH CDR2, and a VH CDR3 having an amino acid sequence of SEQ ID NOs:115, 116, and 117, respectively, and (ii) a VL comprising a VL CDR1, VL CDR2, and VL CDR3 having an amino acid sequence of SEQ ID NOs:118, 119, and 120, respectively. In one embodiment, the first antigen binding domain that specifically binds CD8, comprises: (i) a VH comprising a VH CDR1, a VH CDR2, and a VH CDR3 having an amino acid sequence of SEQ ID NOs:121, 122, and 123, respectively, and (ii) a VL comprising a VL CDR1, VL CDR2, and VL CDR3 having an amino acid sequence of SEQ ID NOs:124, 125, and 126, respectively. In one embodiment, the first antigen binding domain that specifically binds CD8, comprises: (i) a VH comprising a VH CDR1, a VH CDR2, and a VH CDR3 having an amino acid sequence of SEQ ID NOs:127, 128, and 129, respectively, and (ii) a VL comprising a VL CDR1, VL CDR2, and VL CDR3 having an amino acid sequence of SEQ ID NOs:130, 131, and 132, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:133; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:134. In one aspect, provided herein is an antibody that binds CD8, comprising a VH having an amino acid sequence of SEQ ID NO:133. In one aspect, provided herein is an antibody that binds CD8, comprising a VL having an amino acid sequence of SEQ ID NO:134. In one aspect, provided herein is an antibody that binds CD8, comprising a VH having an amino acid sequence of SEQ ID NO:133, and a VL having an amino acid sequence of SEQ ID NO:134. In one aspect, provided herein is an antibody that binds CD8, comprising a heavy chain having an amino acid sequence of SEQ ID NO:135. In one aspect, provided herein is an antibody that binds CD8, comprising a light chain having an amino acid sequence of SEQ ID NO:136. In one aspect, provided herein is an antibody that binds CD8, comprising a heavy chain having an amino acid sequence of SEQ ID NO:135, and a light chain having an amino acid sequence of SEQ ID NO:136. In one aspect, provided herein is an antibody that binds CD8, comprising a VH having an amino acid sequence having at least 95% identity to an amino acid sequence of SEQ ID NO:133. In one aspect, provided herein is an antibody that binds CD8, comprising a VL having an amino acid sequence having at least 95% identity to an amino acid sequence of SEQ ID NO:134. In one aspect, provided herein is an antibody that binds CD8, comprising a VH having an amino acid sequence having at least 95% identity to an amino acid sequence of SEQ ID NO:133, and a VL having an amino acid sequence having at least 95% identity to an amino acid sequence of SEQ ID NO:134. In one aspect, provided herein is an antibody that binds CD8, comprising a heavy chain having an amino acid sequence having at least 95% identity to an amino acid sequence of SEQ ID NO:135. In one aspect, provided herein is an antibody that binds CD8, comprising a light chain having an amino acid sequence having at least 95% identity to an amino acid sequence of SEQ ID NO:136. In one aspect, provided herein is an antibody that binds CD8, comprising a heavy chain having an amino acid sequence having at least 95% identity to an amino acid sequence of SEQ ID NO:135, and a light chain having an amino acid sequence having at least 95% identity to an amino acid sequence of SEQ ID NO:136.

In one embodiment, the first antigen binding domain that specifically binds CD8, comprises: (i) a VH comprising a VH CDR1, a VH CDR2, and a VH CDR3 having an amino acid sequence of SEQ ID NOs:137, 138, and 139, respectively, and (ii) a VL comprising a VL CDR1, VL CDR2, and VL CDR3 having an amino acid sequence of SEQ ID NOs:140, 141, and 142, respectively. In one embodiment, the first antigen binding domain that specifically binds CD8, comprises: (i) a VH comprising a VH CDR1, a VH CDR2, and a VH CDR3 having an amino acid sequence of SEQ ID NOs:143, 144, and 145, respectively, and (ii) a VL comprising a VL CDR1, VL CDR2, and VL CDR3 having an amino acid sequence of SEQ ID NOs:146, 147, and 148, respectively. In one embodiment, the first antigen binding domain that specifically binds CD8, comprises: (i) a VH comprising a VH CDR1, a VH CDR2, and a VH CDR3 having an amino acid sequence of SEQ ID NOs:149, 150, and 151, respectively, and (ii) a VL comprising a VL CDR1, VL CDR2, and VL CDR3 having an amino acid sequence of SEQ ID NOs:152, 153, and 154, respectively. In one embodiment, the first antigen binding domain that specifically binds CD8, comprises: (i) a VH comprising a VH CDR1, a VH CDR2, and a VH CDR3 having an amino acid sequence of SEQ ID NOs:155, 156, and 157, respectively, and (ii) a VL comprising a VL CDR1, VL CDR2, and VL CDR3 having an amino acid sequence of SEQ ID NOs:158, 159, and 160, respectively. In one embodiment, the first antigen binding domain that specifically binds CD8, comprises: (i) a VH comprising a VH CDR1, a VH CDR2, and a VH CDR3 having an amino acid sequence of SEQ ID NOs:161, 162, and 163, respectively, and (ii) a VL comprising a VL CDR1, VL CDR2, and VL CDR3 having an amino acid sequence of SEQ ID NOs:164, 165, and 166, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:167; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:168. In one aspect, provided herein is an antibody that binds CD8, comprising a VH having an amino acid sequence of SEQ ID NO:167. In one aspect, provided herein is an antibody that binds CD8, comprising a VL having an amino acid sequence of SEQ ID NO:168. In one aspect, provided herein is an antibody that binds CD8, comprising a VH having an amino acid sequence of SEQ ID NO:167, and a VL having an amino acid sequence of SEQ ID NO:168. In one aspect, provided herein is an antibody that binds CD8, comprising a heavy chain having an amino acid sequence of SEQ ID NO:169. In one aspect, provided herein is an antibody that binds CD8, comprising a light chain having an amino acid sequence of SEQ ID NO:170. In one aspect, provided herein is an antibody that binds CD8, comprising a heavy chain having an amino acid sequence of SEQ ID NO:169, and a light chain having an amino acid sequence of SEQ ID NO:170. In one aspect, provided herein is an antibody that binds CD8, comprising a VH having an amino acid sequence having at least 95% identity to an amino acid sequence of SEQ ID NO:167. In one aspect, provided herein is an antibody that binds CD8, comprising a VL having an amino acid sequence having at least 95% identity to an amino acid sequence of SEQ ID NO:168. In one aspect, provided herein is an antibody that binds CD8, comprising a VH having an amino acid sequence having at least 95% identity to an amino acid sequence of SEQ ID NO:167, and a VL having an amino acid sequence having at least 95% identity to an amino acid sequence of SEQ ID NO:168. In one aspect, provided herein is an antibody that binds CD8, comprising a heavy chain having an amino acid sequence having at least 95% identity to an amino acid sequence of SEQ ID NO:169. In one aspect, provided herein is an antibody that binds CD8, comprising a light chain having an amino acid sequence having at least 95% identity to an amino acid sequence of SEQ ID NO:170. In one aspect, provided herein is an antibody that binds CD8, comprising a heavy chain having an amino acid sequence having at least 95% identity to an amino acid sequence of SEQ ID NO:169, and a light chain having an amino acid sequence having at least 95% identity to an amino acid sequence of SEQ ID NO:170.

In one embodiment, the first antigen binding domain that specifically binds CD8, comprises: (i) a VH comprising a VH CDR1, a VH CDR2, and a VH CDR3 having an amino acid sequence of SEQ ID NOs:171, 172, and 173, respectively, and (ii) a VL comprising a VL CDR1, VL CDR2, and VL CDR3 having an amino acid sequence of SEQ ID NOs:174, 175, and 176, respectively. In one embodiment, the first antigen binding domain that specifically binds CD8, comprises: (i) a VH comprising a VH CDR1, a VH CDR2, and a VH CDR3 having an amino acid sequence of SEQ ID NOs:177, 178, and 179, respectively, and (ii) a VL comprising a VL CDR1, VL CDR2, and VL CDR3 having an amino acid sequence of SEQ ID NOs:180, 181, and 182, respectively. In one embodiment, the first antigen binding domain that specifically binds CD8, comprises: (i) a VH comprising a VH CDR1, a VH CDR2, and a VH CDR3 having an amino acid sequence of SEQ ID NOs:183, 184, and 185, respectively, and (ii) a VL comprising a VL CDR1, VL CDR2, and VL CDR3 having an amino acid sequence of SEQ ID NOs:186, 187, and 188, respectively. In one embodiment, the first antigen binding domain that specifically binds CD8, comprises: (i) a VH comprising a VH CDR1, a VH CDR2, and a VH CDR3 having an amino acid sequence of SEQ ID NOs:189, 190, and 191, respectively, and (ii) a VL comprising a VL CDR1, VL CDR2, and VL CDR3 having an amino acid sequence of SEQ ID NOs:192, 193, and 194, respectively. In one embodiment, the first antigen binding domain that specifically binds CD8, comprises: (i) a VH comprising a VH CDR1, a VH CDR2, and a VH CDR3 having an amino acid sequence of SEQ ID NOs:195, 196, and 197, respectively, and (ii) a VL comprising a VL CDR1, VL CDR2, and VL CDR3 having an amino acid sequence of SEQ ID NOs:198, 199, and 200, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:201; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:202. In one aspect, provided herein is an antibody that binds CD8, comprising a VH having an amino acid sequence of SEQ ID NO:201. In one aspect, provided herein is an antibody that binds CD8, comprising a VL having an amino acid sequence of SEQ ID NO:202. In one aspect, provided herein is an antibody that binds CD8, comprising a VH having an amino acid sequence of SEQ ID NO:201, and a VL having an amino acid sequence of SEQ ID NO:202. In one aspect, provided herein is an antibody that binds CD8, comprising a heavy chain having an amino acid sequence of SEQ ID NO:203. In one aspect, provided herein is an antibody that binds CD8, comprising a light chain having an amino acid sequence of SEQ ID NO:204. In one aspect, provided herein is an antibody that binds CD8, comprising a heavy chain having an amino acid sequence of SEQ ID NO:203, and a light chain having an amino acid sequence of SEQ ID NO:204. In one aspect, provided herein is an antibody that binds CD8, comprising a VH having an amino acid sequence having at least 95% identity to an amino acid sequence of SEQ ID NO:201. In one aspect, provided herein is an antibody that binds CD8, comprising a VL having an amino acid sequence having at least 95% identity to an amino acid sequence of SEQ ID NO:202. In one aspect, provided herein is an antibody that binds CD8, comprising a VH having an amino acid sequence having at least 95% identity to an amino acid sequence of SEQ ID NO:201, and a VL having an amino acid sequence having at least 95% identity to an amino acid sequence of SEQ ID NO:202. In one aspect, provided herein is an antibody that binds CD8, comprising a heavy chain having an amino acid sequence having at least 95% identity to an amino acid sequence of SEQ ID NO:203. In one aspect, provided herein is an antibody that binds CD8, comprising a light chain having an amino acid sequence having at least 95% identity to an amino acid sequence of SEQ ID NO:204. In one aspect, provided herein is an antibody that binds CD8, comprising a heavy chain having an amino acid sequence having at least 95% identity to an amino acid sequence of SEQ ID NO:203, and a light chain having an amino acid sequence having at least 95% identity to an amino acid sequence of SEQ ID NO:204.

In one embodiment, the first antigen binding domain that specifically binds CD8, comprises: (i) a VH comprising a VH CDR1, a VH CDR2, and a VH CDR3 having an amino acid sequence of SEQ ID NOs:205, 206, and 207, respectively, and (ii) a VL comprising a VL CDR1, VL CDR2, and VL CDR3 having an amino acid sequence of SEQ ID NOs:208, 209, and 210, respectively. In one embodiment, the first antigen binding domain that specifically binds CD8, comprises: (i) a VH comprising a VH CDR1, a VH CDR2, and a VH CDR3 having an amino acid sequence of SEQ ID NOs:211, 212, and 213, respectively, and (ii) a VL comprising a VL CDR1, VL CDR2, and VL CDR3 having an amino acid sequence of SEQ ID NOs:214, 215, and 216, respectively. In one embodiment, the first antigen binding domain that specifically binds CD8, comprises: (i) a VH comprising a VH CDR1, a VH CDR2, and a VH CDR3 having an amino acid sequence of SEQ ID NOs:217, 218, and 219, respectively, and (ii) a VL comprising a VL CDR1, VL CDR2, and VL CDR3 having an amino acid sequence of SEQ ID NOs:220, 221, and 222, respectively. In one embodiment, the first antigen binding domain that specifically binds CD8, comprises: (i) a VH comprising a VH CDR1, a VH CDR2, and a VH CDR3 having an amino acid sequence of SEQ ID NOs:223, 224, and 225, respectively, and (ii) a VL comprising a VL CDR1, VL CDR2, and VL CDR3 having an amino acid sequence of SEQ ID NOs:226, 227, and 228, respectively. In one embodiment, the first antigen binding domain that specifically binds CD8, comprises: (i) a VH comprising a VH CDR1, a VH CDR2, and a VH CDR3 having an amino acid sequence of SEQ ID NOs:229, 230, and 231, respectively, and (ii) a VL comprising a VL CDR1, VL CDR2, and VL CDR3 having an amino acid sequence of SEQ ID NOs:232, 233, and 234, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:235; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:236. In one aspect, provided herein is an antibody that binds CD8, comprising a VH having an amino acid sequence of SEQ ID NO:235. In one aspect, provided herein is an antibody that binds CD8, comprising a VL having an amino acid sequence of SEQ ID NO:236. In one aspect, provided herein is an antibody that binds CD8, comprising a VH having an amino acid sequence of SEQ ID NO:235, and a VL having an amino acid sequence of SEQ ID NO:236. In one aspect, provided herein is an antibody that binds CD8, comprising a heavy chain having an amino acid sequence of SEQ ID NO:237. In one aspect, provided herein is an antibody that binds CD8, comprising a light chain having an amino acid sequence of SEQ ID NO:238. In one aspect, provided herein is an antibody that binds CD8, comprising a heavy chain having an amino acid sequence of SEQ ID NO:237, and a light chain having an amino acid sequence of SEQ ID NO:238. In one aspect, provided herein is an antibody that binds CD8, comprising a VH having an amino acid sequence having at least 95% identity to an amino acid sequence of SEQ ID NO:235. In one aspect, provided herein is an antibody that binds CD8, comprising a VL having an amino acid sequence having at least 95% identity to an amino acid sequence of SEQ ID NO:236. In one aspect, provided herein is an antibody that binds CD8, comprising a VH having an amino acid sequence having at least 95% identity to an amino acid sequence of SEQ ID NO:235, and a VL having an amino acid sequence having at least 95% identity to an amino acid sequence of SEQ ID NO:236. In one aspect, provided herein is an antibody that binds CD8, comprising a heavy chain having an amino acid sequence having at least 95% identity to an amino acid sequence of SEQ ID NO:237. In one aspect, provided herein is an antibody that binds CD8, comprising a light chain having an amino acid sequence having at least 95% identity to an amino acid sequence of SEQ ID NO:238. In one aspect, provided herein is an antibody that binds CD8, comprising a heavy chain having an amino acid sequence having at least 95% identity to an amino acid sequence of SEQ ID NO:237, and a light chain having an amino acid sequence having at least 95% identity to an amino acid sequence of SEQ ID NO:238.

In one embodiment, the first antigen binding domain that specifically binds CD8, comprises: (i) a VH comprising a VH CDR1, a VH CDR2, and a VH CDR3 having an amino acid sequence of SEQ ID NOs:239, 240, and 241, respectively, and (ii) a VL comprising a VL CDR1, VL CDR2, and VL CDR3 having an amino acid sequence of SEQ ID NOs:242, 243, and 244, respectively. In one embodiment, the first antigen binding domain that specifically binds CD8, comprises: (i) a VH comprising a VH CDR1, a VH CDR2, and a VH CDR3 having an amino acid sequence of SEQ ID NOs:245, 246, and 247, respectively, and (ii) a VL comprising a VL CDR1, VL CDR2, and VL CDR3 having an amino acid sequence of SEQ ID NOs:248, 249, and 250, respectively. In one embodiment, the first antigen binding domain that specifically binds CD8, comprises: (i) a VH comprising a VH CDR1, a VH CDR2, and a VH CDR3 having an amino acid sequence of SEQ ID NOs:251, 252, and 253, respectively, and (ii) a VL comprising a VL CDR1, VL CDR2, and VL CDR3 having an amino acid sequence of SEQ ID NOs:254, 255, and 256, respectively. In one embodiment, the first antigen binding domain that specifically binds CD8, comprises: (i) a VH comprising a VH CDR1, a VH CDR2, and a VH CDR3 having an amino acid sequence of SEQ ID NOs:257, 258, and 259, respectively, and (ii) a VL comprising a VL CDR1, VL CDR2, and VL CDR3 having an amino acid sequence of SEQ ID NOs:260, 261, and 262, respectively. In one embodiment, the first antigen binding domain that specifically binds CD8, comprises: (i) a VH comprising a VH CDR1, a VH CDR2, and a VH CDR3 having an amino acid sequence of SEQ ID NOs:263, 264, and 265, respectively, and (ii) a VL comprising a VL CDR1, VL CDR2, and VL CDR3 having an amino acid sequence of SEQ ID NOs:266, 267, and 268, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:269; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:270. In one aspect, provided herein is an antibody that binds CD8, comprising a VH having an amino acid sequence of SEQ ID NO:269. In one aspect, provided herein is an antibody that binds CD8, comprising a VL having an amino acid sequence of SEQ ID NO:270. In one aspect, provided herein is an antibody that binds CD8, comprising a VH having an amino acid sequence of SEQ ID NO:269, and a VL having an amino acid sequence of SEQ ID NO:270. In one aspect, provided herein is an antibody that binds CD8, comprising a heavy chain having an amino acid sequence of SEQ ID NO:271. In one aspect, provided herein is an antibody that binds CD8, comprising a light chain having an amino acid sequence of SEQ ID NO:272. In one aspect, provided herein is an antibody that binds CD8, comprising a heavy chain having an amino acid sequence of SEQ ID NO:271, and a light chain having an amino acid sequence of SEQ ID NO:272. In one aspect, provided herein is an antibody that binds CD8, comprising a VH having an amino acid sequence having at least 95% identity to an amino acid sequence of SEQ ID NO:269. In one aspect, provided herein is an antibody that binds CD8, comprising a VL having an amino acid sequence having at least 95% identity to an amino acid sequence of SEQ ID NO:270. In one aspect, provided herein is an antibody that binds CD8, comprising a VH having an amino acid sequence having at least 95% identity to an amino acid sequence of SEQ ID NO:269, and a VL having an amino acid sequence having at least 95% identity to an amino acid sequence of SEQ ID NO:270. In one aspect, provided herein is an antibody that binds CD8, comprising a heavy chain having an amino acid sequence having at least 95% identity to an amino acid sequence of SEQ ID NO:271. In one aspect, provided herein is an antibody that binds CD8, comprising a light chain having an amino acid sequence having at least 95% identity to an amino acid sequence of SEQ ID NO:272. In one aspect, provided herein is an antibody that binds CD8, comprising a heavy chain having an amino acid sequence having at least 95% identity to an amino acid sequence of SEQ ID NO:271, and a light chain having an amino acid sequence having at least 95% identity to an amino acid sequence of SEQ ID NO:272.

In one embodiment, the first antigen binding domain that specifically binds CD8, comprises: (i) a VH comprising a VH CDR1, a VH CDR2, and a VH CDR3 having an amino acid sequence of SEQ ID NOs:273, 274, and 275, respectively, and (ii) a VL comprising a VL CDR1, VL CDR2, and VL CDR3 having an amino acid sequence of SEQ ID NOs:276, 277, and 278, respectively. In one embodiment, the first antigen binding domain that specifically binds CD8, comprises: (i) a VH comprising a VH CDR1, a VH CDR2, and a VH CDR3 having an amino acid sequence of SEQ ID NOs:279, 280, and 281, respectively, and (ii) a VL comprising a VL CDR1, VL CDR2, and VL CDR3 having an amino acid sequence of SEQ ID NOs:282, 283, and 284, respectively. In one embodiment, the first antigen binding domain that specifically binds CD8, comprises: (i) a VH comprising a VH CDR1, a VH CDR2, and a VH CDR3 having an amino acid sequence of SEQ ID NOs:285, 286, and 287, respectively, and (ii) a VL comprising a VL CDR1, VL CDR2, and VL CDR3 having an amino acid sequence of SEQ ID NOs:288, 289, and 290, respectively. In one embodiment, the first antigen binding domain that specifically binds CD8, comprises: (i) a VH comprising a VH CDR1, a VH CDR2, and a VH CDR3 having an amino acid sequence of SEQ ID NOs:291, 292, and 293, respectively, and (ii) a VL comprising a VL CDR1, VL CDR2, and VL CDR3 having an amino acid sequence of SEQ ID NOs:294, 295, and 296, respectively. In one embodiment, the first antigen binding domain that specifically binds CD8, comprises: (i) a VH comprising a VH CDR1, a VH CDR2, and a VH CDR3 having an amino acid sequence of SEQ ID NOs:297, 298, and 299, respectively, and (ii) a VL comprising a VL CDR1, VL CDR2, and VL CDR3 having an amino acid sequence of SEQ ID NOs:300, 301, and 302, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:303; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:304. In one aspect, provided herein is an antibody that binds CD8, comprising a VH having an amino acid sequence of SEQ ID NO:303. In one aspect, provided herein is an antibody that binds CD8, comprising a VL having an amino acid sequence of SEQ ID NO:304. In one aspect, provided herein is an antibody that binds CD8, comprising a VH having an amino acid sequence of SEQ ID NO:303, and a VL having an amino acid sequence of SEQ ID NO:304. In one aspect, provided herein is an antibody that binds CD8, comprising a heavy chain having an amino acid sequence of SEQ ID NO:305. In one aspect, provided herein is an antibody that binds CD8, comprising a light chain having an amino acid sequence of SEQ ID NO:306. In one aspect, provided herein is an antibody that binds CD8, comprising a heavy chain having an amino acid sequence of SEQ ID NO:305, and a light chain having an amino acid sequence of SEQ ID NO:306. In one aspect, provided herein is an antibody that binds CD8, comprising a VH having an amino acid sequence having at least 95% identity to an amino acid sequence of SEQ ID NO:303. In one aspect, provided herein is an antibody that binds CD8, comprising a VL having an amino acid sequence having at least 95% identity to an amino acid sequence of SEQ ID NO:304. In one aspect, provided herein is an antibody that binds CD8, comprising a VH having an amino acid sequence having at least 95% identity to an amino acid sequence of SEQ ID NO:303, and a VL having an amino acid sequence having at least 95% identity to an amino acid sequence of SEQ ID NO:304. In one aspect, provided herein is an antibody that binds CD8, comprising a heavy chain having an amino acid sequence having at least 95% identity to an amino acid sequence of SEQ ID NO:305. In one aspect, provided herein is an antibody that binds CD8, comprising a light chain having an amino acid sequence having at least 95% identity to an amino acid sequence of SEQ ID NO:306. In one aspect, provided herein is an antibody that binds CD8, comprising a heavy chain having an amino acid sequence having at least 95% identity to an amino acid sequence of SEQ ID NO:305, and a light chain having an amino acid sequence having at least 95% identity to an amino acid sequence of SEQ ID NO:306.

In one embodiment, the first antigen binding domain that specifically binds CD8, comprises: (i) a VH comprising a VH CDR1, a VH CDR2, and a VH CDR3 having an amino acid sequence of SEQ ID NOs:307, 308, and 309, respectively, and (ii) a VL comprising a VL CDR1, VL CDR2, and VL CDR3 having an amino acid sequence of SEQ ID NOs:310, 311, and 312, respectively. In one embodiment, the first antigen binding domain that specifically binds CD8, comprises: (i) a VH comprising a VH CDR1, a VH CDR2, and a VH CDR3 having an amino acid sequence of SEQ ID NOs:313, 314, and 315, respectively, and (ii) a VL comprising a VL CDR1, VL CDR2, and VL CDR3 having an amino acid sequence of SEQ ID NOs:316, 317, and 318, respectively. In one embodiment, the first antigen binding domain that specifically binds CD8, comprises: (i) a VH comprising a VH CDR1, a VH CDR2, and a VH CDR3 having an amino acid sequence of SEQ ID NOs:319, 320, and 321, respectively, and (ii) a VL comprising a VL CDR1, VL CDR2, and VL CDR3 having an amino acid sequence of SEQ ID NOs:322, 323, and 324, respectively. In one embodiment, the first antigen binding domain that specifically binds CD8, comprises: (i) a VH comprising a VH CDR1, a VH CDR2, and a VH CDR3 having an amino acid sequence of SEQ ID NOs:325, 326, and 327, respectively, and (ii) a VL comprising a VL CDR1, VL CDR2, and VL CDR3 having an amino acid sequence of SEQ ID NOs:328, 329, and 330, respectively. In one embodiment, the first antigen binding domain that specifically binds CD8, comprises: (i) a VH comprising a VH CDR1, a VH CDR2, and a VH CDR3 having an amino acid sequence of SEQ ID NOs:331, 332, and 333, respectively, and (ii) a VL comprising a VL CDR1, VL CDR2, and VL CDR3 having an amino acid sequence of SEQ ID NOs:334, 335, and 336, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:337; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:338. In one aspect, provided herein is an antibody that binds CD8, comprising a VH having an amino acid sequence of SEQ ID NO:337. In one aspect, provided herein is an antibody that binds CD8, comprising a VL having an amino acid sequence of SEQ ID NO:338. In one aspect, provided herein is an antibody that binds CD8, comprising a VH having an amino acid sequence of SEQ ID NO:337, and a VL having an amino acid sequence of SEQ ID NO:338. In one aspect, provided herein is an antibody that binds CD8, comprising a heavy chain having an amino acid sequence of SEQ ID NO:339. In one aspect, provided herein is an antibody that binds CD8, comprising a light chain having an amino acid sequence of SEQ ID NO:340. In one aspect, provided herein is an antibody that binds CD8, comprising a heavy chain having an amino acid sequence of SEQ ID NO:339, and a light chain having an amino acid sequence of SEQ ID NO:340. In one aspect, provided herein is an antibody that binds CD8, comprising a VH having an amino acid sequence having at least 95% identity to an amino acid sequence of SEQ ID NO:337. In one aspect, provided herein is an antibody that binds CD8, comprising a VL having an amino acid sequence having at least 95% identity to an amino acid sequence of SEQ ID NO:338. In one aspect, provided herein is an antibody that binds CD8, comprising a VH having an amino acid sequence having at least 95% identity to an amino acid sequence of SEQ ID NO:337, and a VL having an amino acid sequence having at least 95% identity to an amino acid sequence of SEQ ID NO:338. In one aspect, provided herein is an antibody that binds CD8, comprising a heavy chain having an amino acid sequence having at least 95% identity to an amino acid sequence of SEQ ID NO:339. In one aspect, provided herein is an antibody that binds CD8, comprising a light chain having an amino acid sequence having at least 95% identity to an amino acid sequence of SEQ ID NO:340. In one aspect, provided herein is an antibody that binds CD8, comprising a heavy chain having an amino acid sequence having at least 95% identity to an amino acid sequence of SEQ ID NO:339, and a light chain having an amino acid sequence having at least 95% identity to an amino acid sequence of SEQ ID NO:340.

In one embodiment, the first antigen binding domain that specifically binds CD8, comprises: (i) a VH comprising a VH CDR1, a VH CDR2, and a VH CDR3 having an amino acid sequence of SEQ ID NOs:341, 342, and 343, respectively, and (ii) a VL comprising a VL CDR1, VL CDR2, and VL CDR3 having an amino acid sequence of SEQ ID NOs:344, 345, and 346, respectively. In one embodiment, the first antigen binding domain that specifically binds CD8, comprises: (i) a VH comprising a VH CDR1, a VH CDR2, and a VH CDR3 having an amino acid sequence of SEQ ID NOs:347, 348, and 349, respectively, and (ii) a VL comprising a VL CDR1, VL CDR2, and VL CDR3 having an amino acid sequence of SEQ ID NOs:350, 351, and 352, respectively. In one embodiment, the first antigen binding domain that specifically binds CD8, comprises: (i) a VH comprising a VH CDR1, a VH CDR2, and a VH CDR3 having an amino acid sequence of SEQ ID NOs:353, 354, and 355, respectively, and (ii) a VL comprising a VL CDR1, VL CDR2, and VL CDR3 having an amino acid sequence of SEQ ID NOs:356, 357, and 358, respectively. In one embodiment, the first antigen binding domain that specifically binds CD8, comprises: (i) a VH comprising a VH CDR1, a VH CDR2, and a VH CDR3 having an amino acid sequence of SEQ ID NOs:359, 360, and 361, respectively, and (ii) a VL comprising a VL CDR1, VL CDR2, and VL CDR3 having an amino acid sequence of SEQ ID NOs:362, 363, and 364, respectively. In one embodiment, the first antigen binding domain that specifically binds CD8, comprises: (i) a VH comprising a VH CDR1, a VH CDR2, and a VH CDR3 having an amino acid sequence of SEQ ID NOs:365, 366, and 367, respectively, and (ii) a VL comprising a VL CDR1, VL CDR2, and VL CDR3 having an amino acid sequence of SEQ ID NOs:368, 369, and 370, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:371; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:372. In one aspect, provided herein is an antibody that binds CD8, comprising a VH having an amino acid sequence of SEQ ID NO:371. In one aspect, provided herein is an antibody that binds CD8, comprising a VL having an amino acid sequence of SEQ ID NO:372. In one aspect, provided herein is an antibody that binds CD8, comprising a VH having an amino acid sequence of SEQ ID NO:371, and a VL having an amino acid sequence of SEQ ID NO:372. In one aspect, provided herein is an antibody that binds CD8, comprising a heavy chain having an amino acid sequence of SEQ ID NO:373. In one aspect, provided herein is an antibody that binds CD8, comprising a light chain having an amino acid sequence of SEQ ID NO:374. In one aspect, provided herein is an antibody that binds CD8, comprising a heavy chain having an amino acid sequence of SEQ ID NO:373, and a light chain having an amino acid sequence of SEQ ID NO:374. In one aspect, provided herein is an antibody that binds CD8, comprising a VH having an amino acid sequence having at least 95% identity to an amino acid sequence of SEQ ID NO:371. In one aspect, provided herein is an antibody that binds CD8, comprising a VL having an amino acid sequence having at least 95% identity to an amino acid sequence of SEQ ID NO:372. In one aspect, provided herein is an antibody that binds CD8, comprising a VH having an amino acid sequence having at least 95% identity to an amino acid sequence of SEQ ID NO:371, and a VL having an amino acid sequence having at least 95% identity to an amino acid sequence of SEQ ID NO:372. In one aspect, provided herein is an antibody that binds CD8, comprising a heavy chain having an amino acid sequence having at least 95% identity to an amino acid sequence of SEQ ID NO:373. In one aspect, provided herein is an antibody that binds CD8, comprising a light chain having an amino acid sequence having at least 95% identity to an amino acid sequence of SEQ ID NO:374. In one aspect, provided herein is an antibody that binds CD8, comprising a heavy chain having an amino acid sequence having at least 95% identity to an amino acid sequence of SEQ ID NO:373, and a light chain having an amino acid sequence having at least 95% identity to an amino acid sequence of SEQ ID NO:374.

In one embodiment, the first antigen binding domain that specifically binds CD8, comprises: (i) a VH comprising a VH CDR1, a VH CDR2, and a VH CDR3 having an amino acid sequence of SEQ ID NOs:375, 376, and 377, respectively, and (ii) a VL comprising a VL CDR1, VL CDR2, and VL CDR3 having an amino acid sequence of SEQ ID NOs:378, 379, and 380, respectively. In one embodiment, the first antigen binding domain that specifically binds CD8, comprises: (i) a VH comprising a VH CDR1, a VH CDR2, and a VH CDR3 having an amino acid sequence of SEQ ID NOs:381, 382, and 383, respectively, and (ii) a VL comprising a VL CDR1, VL CDR2, and VL CDR3 having an amino acid sequence of SEQ ID NOs:384, 385, and 386, respectively. In one embodiment, the first antigen binding domain that specifically binds CD8, comprises: (i) a VH comprising a VH CDR1, a VH CDR2, and a VH CDR3 having an amino acid sequence of SEQ ID NOs:387, 388, and 389, respectively, and (ii) a VL comprising a VL CDR1, VL CDR2, and VL CDR3 having an amino acid sequence of SEQ ID NOs:390, 391, and 392, respectively. In one embodiment, the first antigen binding domain that specifically binds CD8, comprises: (i) a VH comprising a VH CDR1, a VH CDR2, and a VH CDR3 having an amino acid sequence of SEQ ID NOs:393, 394, and 395, respectively, and (ii) a VL comprising a VL CDR1, VL CDR2, and VL CDR3 having an amino acid sequence of SEQ ID NOs:396, 397, and 398, respectively. In one embodiment, the first antigen binding domain that specifically binds CD8, comprises: (i) a VH comprising a VH CDR1, a VH CDR2, and a VH CDR3 having an amino acid sequence of SEQ ID NOs:399, 400, and 401, respectively, and (ii) a VL comprising a VL CDR1, VL CDR2, and VL CDR3 having an amino acid sequence of SEQ ID NOs:402, 403, and 404, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:405; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:406. In one aspect, provided herein is an antibody that binds CD8, comprising a VH having an amino acid sequence of SEQ ID NO:405. In one embodiment, the first antigen binding domain that specifically binds CD8, comprises a VL having an amino acid sequence of SEQ ID NO:406. In one embodiment, the first antigen binding domain that specifically binds CD8, comprises a VH having an amino acid sequence of SEQ ID NO:405, and a VL having an amino acid sequence of SEQ ID NO:406. In one embodiment, the first antigen binding domain that specifically binds CD8, comprises a heavy chain having an amino acid sequence of SEQ ID NO:407. In one embodiment, the first antigen binding domain that specifically binds CD8, comprises a light chain having an amino acid sequence of SEQ ID NO:408. In one embodiment, the first antigen binding domain that specifically binds CD8, comprises a heavy chain having an amino acid sequence of SEQ ID NO:407, and a light chain having an amino acid sequence of SEQ ID NO:408. In one embodiment, the first antigen binding domain that specifically binds CD8, comprises a VH having an amino acid sequence having at least 95% identity to an amino acid sequence of SEQ ID NO:405. In one embodiment, the first antigen binding domain that specifically binds CD8, comprises a VL having an amino acid sequence having at least 95% identity to an amino acid sequence of SEQ ID NO:406. In one embodiment, the first antigen binding domain that specifically binds CD8, comprises a VH having an amino acid sequence having at least 95% identity to an amino acid sequence of SEQ ID NO:405, and a VL having an amino acid sequence having at least 95% identity to an amino acid sequence of SEQ ID NO:406. In one embodiment, the first antigen binding domain that specifically binds CD8, comprises a heavy chain having an amino acid sequence having at least 95% identity to an amino acid sequence of SEQ ID NO:407. In one embodiment, the first antigen binding domain that specifically binds CD8, comprises a light chain having an amino acid sequence having at least 95% identity to an amino acid sequence of SEQ ID NO:408. In one embodiment, the first antigen binding domain that specifically binds CD8, comprises a heavy chain having an amino acid sequence having at least 95% identity to an amino acid sequence of SEQ ID NO:407, and a light chain having an amino acid sequence having at least 95% identity to an amino acid sequence of SEQ ID NO:408.

In one embodiment, the first antigen binding domain that specifically binds CD8, comprises: (i) a VH comprising a VH CDR1, a VH CDR2, and a VH CDR3 having an amino acid sequence of SEQ ID NOs:409, 410, and 411, respectively, and (ii) a VL comprising a VL CDR1, VL CDR2, and VL CDR3 having an amino acid sequence of SEQ ID NOs:412, 413, and 414, respectively. In one embodiment, the first antigen binding domain that specifically binds CD8, comprises: (i) a VH comprising a VH CDR1, a VH CDR2, and a VH CDR3 having an amino acid sequence of SEQ ID NOs:415, 416, and 417, respectively, and (ii) a VL comprising a VL CDR1, VL CDR2, and VL CDR3 having an amino acid sequence of SEQ ID NOs:418, 419, and 420, respectively. In one embodiment, the first antigen binding domain that specifically binds CD8, comprises: (i) a VH comprising a VH CDR1, a VH CDR2, and a VH CDR3 having an amino acid sequence of SEQ ID NOs:421, 422, and 423, respectively, and (ii) a VL comprising a VL CDR1, VL CDR2, and VL CDR3 having an amino acid sequence of SEQ ID NOs:424, 425, and 426, respectively. In one embodiment, the first antigen binding domain that specifically binds CD8, comprises: (i) a VH comprising a VH CDR1, a VH CDR2, and a VH CDR3 having an amino acid sequence of SEQ ID NOs:427, 428, and 429, respectively, and (ii) a VL comprising a VL CDR1, VL CDR2, and VL CDR3 having an amino acid sequence of SEQ ID NOs:430, 431, and 432, respectively. In one embodiment, the first antigen binding domain that specifically binds CD8, comprises: (i) a VH comprising a VH CDR1, a VH CDR2, and a VH CDR3 having an amino acid sequence of SEQ ID NOs:433, 434, and 435, respectively, and (ii) a VL comprising a VL CDR1, VL CDR2, and VL CDR3 having an amino acid sequence of SEQ ID NOs:436, 437, and 438, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:439; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:440. In one embodiment, the first antigen binding domain that specifically binds CD8, comprises a VH having an amino acid sequence of SEQ ID NO:439. In one embodiment, the first antigen binding domain that specifically binds CD8, comprises a VL having an amino acid sequence of SEQ ID NO:440. In one embodiment, the first antigen binding domain that specifically binds CD8, comprises a VH having an amino acid sequence of SEQ ID NO:439, and a VL having an amino acid sequence of SEQ ID NO:440. In one embodiment, the first antigen binding domain that specifically binds CD8, comprises a heavy chain having an amino acid sequence of SEQ ID NO:441. In one embodiment, the first antigen binding domain that specifically binds CD8, comprises a light chain having an amino acid sequence of SEQ ID NO:442. In one embodiment, the first antigen binding domain that specifically binds CD8, comprises a heavy chain having an amino acid sequence of SEQ ID NO:441, and a light chain having an amino acid sequence of SEQ ID NO:442. In one embodiment, the first antigen binding domain that specifically binds CD8, comprises a VH having an amino acid sequence having at least 95% identity to an amino acid sequence of SEQ ID NO:439. In one embodiment, the first antigen binding domain that specifically binds CD8, comprises a VL having an amino acid sequence having at least 95% identity to an amino acid sequence of SEQ ID NO:440. In one embodiment, the first antigen binding domain that specifically binds CD8, comprises a VH having an amino acid sequence having at least 95% identity to an amino acid sequence of SEQ ID NO:439, and a VL having an amino acid sequence having at least 95% identity to an amino acid sequence of SEQ ID NO:440. In one embodiment, the first antigen binding domain that specifically binds CD8, comprises a heavy chain having an amino acid sequence having at least 95% identity to an amino acid sequence of SEQ ID NO:441. In one embodiment, the first antigen binding domain that specifically binds CD8, comprises a light chain having an amino acid sequence having at least 95% identity to an amino acid sequence of SEQ ID NO:442. In one embodiment, the first antigen binding domain that specifically binds CD8, comprises a heavy chain having an amino acid sequence having at least 95% identity to an amino acid sequence of SEQ ID NO:441, and a light chain having an amino acid sequence having at least 95% identity to an amino acid sequence of SEQ ID NO:442.

In one embodiment, the first antigen binding domain that specifically binds CD8, comprises: (i) a VH comprising a VH CDR1, a VH CDR2, and a VH CDR3 having an amino acid sequence of SEQ ID NOs:443, 444, and 445, respectively, and (ii) a VL comprising a VL CDR1, VL CDR2, and VL CDR3 having an amino acid sequence of SEQ ID NOs:446, 447, and 448, respectively. In one embodiment, the first antigen binding domain that specifically binds CD8, comprises: (i) a VH comprising a VH CDR1, a VH CDR2, and a VH CDR3 having an amino acid sequence of SEQ ID NOs:449, 450, and 451, respectively, and (ii) a VL comprising a VL CDR1, VL CDR2, and VL CDR3 having an amino acid sequence of SEQ ID NOs:452, 453, and 454, respectively. In one embodiment, the first antigen binding domain that specifically binds CD8, comprises: (i) a VH comprising a VH CDR1, a VH CDR2, and a VH CDR3 having an amino acid sequence of SEQ ID NOs:455, 456, and 457, respectively, and (ii) a VL comprising a VL CDR1, VL CDR2, and VL CDR3 having an amino acid sequence of SEQ ID NOs:458, 459, and 460, respectively. In one embodiment, the first antigen binding domain that specifically binds CD8, comprises: (i) a VH comprising a VH CDR1, a VH CDR2, and a VH CDR3 having an amino acid sequence of SEQ ID NOs:461, 462, and 463, respectively, and (ii) a VL comprising a VL CDR1, VL CDR2, and VL CDR3 having an amino acid sequence of SEQ ID NOs:464, 465, and 466, respectively. In one embodiment, the first antigen binding domain that specifically binds CD8, comprises: (i) a VH comprising a VH CDR1, a VH CDR2, and a VH CDR3 having an amino acid sequence of SEQ ID NOs:467, 468, and 469, respectively, and (ii) a VL comprising a VL CDR1, VL CDR2, and VL CDR3 having an amino acid sequence of SEQ ID NOs:470, 471, and 472, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:473; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:474. In one embodiment, the first antigen binding domain that specifically binds CD8, comprises a VH having an amino acid sequence of SEQ ID NO:473. In one embodiment, the first antigen binding domain that specifically binds CD8, comprises a VL having an amino acid sequence of SEQ ID NO:474. In one embodiment, the first antigen binding domain that specifically binds CD8, comprises a VH having an amino acid sequence of SEQ ID NO:473, and a VL having an amino acid sequence of SEQ ID NO:474. In one embodiment, the first antigen binding domain that specifically binds CD8, comprises a heavy chain having an amino acid sequence of SEQ ID NO:475. In one embodiment, the first antigen binding domain that specifically binds CD8, comprises a light chain having an amino acid sequence of SEQ ID NO:476. In one embodiment, the first antigen binding domain that specifically binds CD8, comprises a heavy chain having an amino acid sequence of SEQ ID NO:475, and a light chain having an amino acid sequence of SEQ ID NO:476. In one embodiment, the first antigen binding domain that specifically binds CD8, comprises a VH having an amino acid sequence having at least 95% identity to an amino acid sequence of SEQ ID NO:473. In one embodiment, the first antigen binding domain that specifically binds CD8, comprises a VL having an amino acid sequence having at least 95% identity to an amino acid sequence of SEQ ID NO:474. In one embodiment, the first antigen binding domain that specifically binds CD8, comprises a VH having an amino acid sequence having at least 95% identity to an amino acid sequence of SEQ ID NO:473, and a VL having an amino acid sequence having at least 95% identity to an amino acid sequence of SEQ ID NO:474. In one embodiment, the first antigen binding domain that specifically binds CD8, comprises a heavy chain having an amino acid sequence having at least 95% identity to an amino acid sequence of SEQ ID NO:475. In one embodiment, the first antigen binding domain that specifically binds CD8, comprises a light chain having an amino acid sequence having at least 95% identity to an amino acid sequence of SEQ ID NO:476. In one embodiment, the first antigen binding domain that specifically binds CD8, comprises a heavy chain having an amino acid sequence having at least 95% identity to an amino acid sequence of SEQ ID NO:475, and a light chain having an amino acid sequence having at least 95% identity to an amino acid sequence of SEQ ID NO:476.

In one embodiment, the first antigen binding domain that specifically binds CD8, comprises: (i) a VH comprising a VH CDR1, a VH CDR2, and a VH CDR3 having an amino acid sequence of SEQ ID NOs:477, 478, and 479, respectively, and (ii) a VL comprising a VL CDR1, VL CDR2, and VL CDR3 having an amino acid sequence of SEQ ID NOs:480, 481, and 482, respectively. In one embodiment, the first antigen binding domain that specifically binds CD8, comprises: (i) a VH comprising a VH CDR1, a VH CDR2, and a VH CDR3 having an amino acid sequence of SEQ ID NOs:483, 484, and 485, respectively, and (ii) a VL comprising a VL CDR1, VL CDR2, and VL CDR3 having an amino acid sequence of SEQ ID NOs:486, 487, and 488, respectively. In one embodiment, the first antigen binding domain that specifically binds CD8, comprises: (i) a VH comprising a VH CDR1, a VH CDR2, and a VH CDR3 having an amino acid sequence of SEQ ID NOs:489, 490, and 491, respectively, and (ii) a VL comprising a VL CDR1, VL CDR2, and VL CDR3 having an amino acid sequence of SEQ ID NOs:492, 493, and 494, respectively. In one embodiment, the first antigen binding domain that specifically binds CD8, comprises: (i) a VH comprising a VH CDR1, a VH CDR2, and a VH CDR3 having an amino acid sequence of SEQ ID NOs:495, 496, and 497, respectively, and (ii) a VL comprising a VL CDR1, VL CDR2, and VL CDR3 having an amino acid sequence of SEQ ID NOs:498, 499, and 500, respectively. In one embodiment, the first antigen binding domain that specifically binds CD8, comprises: (i) a VH comprising a VH CDR1, a VH CDR2, and a VH CDR3 having an amino acid sequence of SEQ ID NOs:501, 502, and 503, respectively, and (ii) a VL comprising a VL CDR1, VL CDR2, and VL CDR3 having an amino acid sequence of SEQ ID NOs:504, 505, and 506, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:507; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:508. In one embodiment, the first antigen binding domain that specifically binds CD8, comprises a VH having an amino acid sequence of SEQ ID NO:507. In one embodiment, the first antigen binding domain that specifically binds CD8, comprises a VL having an amino acid sequence of SEQ ID NO:508. In one embodiment, the first antigen binding domain that specifically binds CD8, comprises a VH having an amino acid sequence of SEQ ID NO:507, and a VL having an amino acid sequence of SEQ ID NO:508. In one embodiment, the first antigen binding domain that specifically binds CD8, comprises a heavy chain having an amino acid sequence of SEQ ID NO:509. In one embodiment, the first antigen binding domain that specifically binds CD8, comprises a light chain having an amino acid sequence of SEQ ID NO:510. In one embodiment, the first antigen binding domain that specifically binds CD8, comprises a heavy chain having an amino acid sequence of SEQ ID NO:509, and a light chain having an amino acid sequence of SEQ ID NO:510. In one embodiment, the first antigen binding domain that specifically binds CD8, comprises a VH having an amino acid sequence having at least 95% identity to an amino acid sequence of SEQ ID NO:507. In one embodiment, the first antigen binding domain that specifically binds CD8, comprises a VL having an amino acid sequence having at least 95% identity to an amino acid sequence of SEQ ID NO:508. In one embodiment, the first antigen binding domain that specifically binds CD8, comprises a VH having an amino acid sequence having at least 95% identity to an amino acid sequence of SEQ ID NO:507, and a VL having an amino acid sequence having at least 95% identity to an amino acid sequence of SEQ ID NO:508. In one embodiment, the first antigen binding domain that specifically binds CD8, comprises a heavy chain having an amino acid sequence having at least 95% identity to an amino acid sequence of SEQ ID NO:509. In one embodiment, the first antigen binding domain that specifically binds CD8, comprises a light chain having an amino acid sequence having at least 95% identity to an amino acid sequence of SEQ ID NO:510. In one embodiment, the first antigen binding domain that specifically binds CD8, comprises a heavy chain having an amino acid sequence having at least 95% identity to an amino acid sequence of SEQ ID NO:509, and a light chain having an amino acid sequence having at least 95% identity to an amino acid sequence of SEQ ID NO:510.

In one embodiment, the first antigen binding domain that specifically binds CD8, comprises: (i) a VH comprising a VH CDR1, a VH CDR2, and a VH CDR3 having an amino acid sequence of SEQ ID NOs:511, 512, and 513, respectively, and (ii) a VL comprising a VL CDR1, VL CDR2, and VL CDR3 having an amino acid sequence of SEQ ID NOs:514, 515, and 516, respectively. In one embodiment, the first antigen binding domain that specifically binds CD8, comprises: (i) a VH comprising a VH CDR1, a VH CDR2, and a VH CDR3 having an amino acid sequence of SEQ ID NOs:517, 518, and 519, respectively, and (ii) a VL comprising a VL CDR1, VL CDR2, and VL CDR3 having an amino acid sequence of SEQ ID NOs:520, 521, and 522, respectively. In one embodiment, the first antigen binding domain that specifically binds CD8, comprises: (i) a VH comprising a VH CDR1, a VH CDR2, and a VH CDR3 having an amino acid sequence of SEQ ID NOs:523, 524, and 525, respectively, and (ii) a VL comprising a VL CDR1, VL CDR2, and VL CDR3 having an amino acid sequence of SEQ ID NOs:526, 527, and 528, respectively. In one embodiment, the first antigen binding domain that specifically binds CD8, comprises: (i) a VH comprising a VH CDR1, a VH CDR2, and a VH CDR3 having an amino acid sequence of SEQ ID NOs:529, 530, and 531, respectively, and (ii) a VL comprising a VL CDR1, VL CDR2, and VL CDR3 having an amino acid sequence of SEQ ID NOs:532, 533, and 534, respectively. In one embodiment, the first antigen binding domain that specifically binds CD8, comprises: (i) a VH comprising a VH CDR1, a VH CDR2, and a VH CDR3 having an amino acid sequence of SEQ ID NOs:535, 536, and 537, respectively, and (ii) a VL comprising a VL CDR1, VL CDR2, and VL CDR3 having an amino acid sequence of SEQ ID NOs:538, 539, and 540, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:541; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:542. In one embodiment, the first antigen binding domain that specifically binds CD8, comprises a VH having an amino acid sequence of SEQ ID NO:541. In one embodiment, the first antigen binding domain that specifically binds CD8, comprises a VL having an amino acid sequence of SEQ ID NO:542. In one embodiment, the first antigen binding domain that specifically binds CD8, comprises a VH having an amino acid sequence of SEQ ID NO:541, and a VL having an amino acid sequence of SEQ ID NO:542. In one embodiment, the first antigen binding domain that specifically binds CD8, comprises a heavy chain having an amino acid sequence of SEQ ID NO:543. In one embodiment, the first antigen binding domain that specifically binds CD8, comprises a light chain having an amino acid sequence of SEQ ID NO:544. In one embodiment, the first antigen binding domain that specifically binds CD8, comprises a heavy chain having an amino acid sequence of SEQ ID NO:543, and a light chain having an amino acid sequence of SEQ ID NO:544. In one embodiment, the first antigen binding domain that specifically binds CD8, comprises a VH having an amino acid sequence having at least 95% identity to an amino acid sequence of SEQ ID NO:541. In one embodiment, the first antigen binding domain that specifically binds CD8, comprises a VL having an amino acid sequence having at least 95% identity to an amino acid sequence of SEQ ID NO:542. In one embodiment, the first antigen binding domain that specifically binds CD8, comprises a VH having an amino acid sequence having at least 95% identity to an amino acid sequence of SEQ ID NO:541, and a VL having an amino acid sequence having at least 95% identity to an amino acid sequence of SEQ ID NO:542. In one embodiment, the first antigen binding domain that specifically binds CD8, comprises a heavy chain having an amino acid sequence having at least 95% identity to an amino acid sequence of SEQ ID NO:543. In one embodiment, the first antigen binding domain that specifically binds CD8, comprises a light chain having an amino acid sequence having at least 95% identity to an amino acid sequence of SEQ ID NO:544. In one embodiment, the first antigen binding domain that specifically binds CD8, comprises a heavy chain having an amino acid sequence having at least 95% identity to an amino acid sequence of SEQ ID NO:543, and a light chain having an amino acid sequence having at least 95% identity to an amino acid sequence of SEQ ID NO:544.

In one embodiment, the first antigen binding domain that specifically binds CD8, comprises: (i) a VH comprising a VH CDR1, a VH CDR2, and a VH CDR3 having an amino acid sequence of SEQ ID NOs:545, 546, and 547, respectively, and (ii) a VL comprising a VL CDR1, VL CDR2, and VL CDR3 having an amino acid sequence of SEQ ID NOs:548, 549, and 550, respectively. In one embodiment, the first antigen binding domain that specifically binds CD8, comprises: (i) a VH comprising a VH CDR1, a VH CDR2, and a VH CDR3 having an amino acid sequence of SEQ ID NOs:551, 552, and 553, respectively, and (ii) a VL comprising a VL CDR1, VL CDR2, and VL CDR3 having an amino acid sequence of SEQ ID NOs:554, 555, and 556, respectively. In one embodiment, the first antigen binding domain that specifically binds CD8, comprises: (i) a VH comprising a VH CDR1, a VH CDR2, and a VH CDR3 having an amino acid sequence of SEQ ID NOs:557, 558, and 559, respectively, and (ii) a VL comprising a VL CDR1, VL CDR2, and VL CDR3 having an amino acid sequence of SEQ ID NOs:560, 561, and 562, respectively. In one embodiment, the first antigen binding domain that specifically binds CD8, comprises: (i) a VH comprising a VH CDR1, a VH CDR2, and a VH CDR3 having an amino acid sequence of SEQ ID NOs:563, 564, and 565, respectively, and (ii) a VL comprising a VL CDR1, VL CDR2, and VL CDR3 having an amino acid sequence of SEQ ID NOs:566, 567, and 568, respectively. In one embodiment, the first antigen binding domain that specifically binds CD8, comprises: (i) a VH comprising a VH CDR1, a VH CDR2, and a VH CDR3 having an amino acid sequence of SEQ ID NOs:569, 570, and 571, respectively, and (ii) a VL comprising a VL CDR1, VL CDR2, and VL CDR3 having an amino acid sequence of SEQ ID NOs:572, 573, and 574, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:575; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:576. In one embodiment, the first antigen binding domain that specifically binds CD8, comprises a VH having an amino acid sequence of SEQ ID NO:575. In one embodiment, the first antigen binding domain that specifically binds CD8, comprises a VL having an amino acid sequence of SEQ ID NO:576. In one embodiment, the first antigen binding domain that specifically binds CD8, comprises a VH having an amino acid sequence of SEQ ID NO:575, and a VL having an amino acid sequence of SEQ ID NO:576. In one embodiment, the first antigen binding domain that specifically binds CD8, comprises a heavy chain having an amino acid sequence of SEQ ID NO:577. In one embodiment, the first antigen binding domain that specifically binds CD8, comprises a light chain having an amino acid sequence of SEQ ID NO:578. In one embodiment, the first antigen binding domain that specifically binds CD8, comprises a heavy chain having an amino acid sequence of SEQ ID NO:577, and a light chain having an amino acid sequence of SEQ ID NO:578. In one embodiment, the first antigen binding domain that specifically binds CD8, comprises a VH having an amino acid sequence having at least 95% identity to an amino acid sequence of SEQ ID NO:575. In one embodiment, the first antigen binding domain that specifically binds CD8, comprises a VL having an amino acid sequence having at least 95% identity to an amino acid sequence of SEQ ID NO:576. In one embodiment, the first antigen binding domain that specifically binds CD8, comprises a VH having an amino acid sequence having at least 95% identity to an amino acid sequence of SEQ ID NO:575, and a VL having an amino acid sequence having at least 95% identity to an amino acid sequence of SEQ ID NO:576. In one embodiment, the first antigen binding domain that specifically binds CD8, comprises a heavy chain having an amino acid sequence having at least 95% identity to an amino acid sequence of SEQ ID NO:577. In one embodiment, the first antigen binding domain that specifically binds CD8, comprises a light chain having an amino acid sequence having at least 95% identity to an amino acid sequence of SEQ ID NO:578. In one embodiment, the first antigen binding domain that specifically binds CD8, comprises a heavy chain having an amino acid sequence having at least 95% identity to an amino acid sequence of SEQ ID NO:577, and a light chain having an amino acid sequence having at least 95% identity to an amino acid sequence of SEQ ID NO:578.

In one embodiment, the first antigen binding domain that specifically binds CD8, comprises: (i) a VH comprising a VH CDR1, a VH CDR2, and a VH CDR3 having an amino acid sequence of SEQ ID NOs:579, 580, and 581, respectively, and (ii) a VL comprising a VL CDR1, VL CDR2, and VL CDR3 having an amino acid sequence of SEQ ID NOs:582, 583, and 584, respectively. In one embodiment, the first antigen binding domain that specifically binds CD8, comprises: (i) a VH comprising a VH CDR1, a VH CDR2, and a VH CDR3 having an amino acid sequence of SEQ ID NOs:585, 586, and 587, respectively, and (ii) a VL comprising a VL CDR1, VL CDR2, and VL CDR3 having an amino acid sequence of SEQ ID NOs:588, 589, and 590, respectively. In one embodiment, the first antigen binding domain that specifically binds CD8, comprises: (i) a VH comprising a VH CDR1, a VH CDR2, and a VH CDR3 having an amino acid sequence of SEQ ID NOs:591, 592, and 593, respectively, and (ii) a VL comprising a VL CDR1, VL CDR2, and VL CDR3 having an amino acid sequence of SEQ ID NOs:594, 595, and 596, respectively. In one embodiment, the first antigen binding domain that specifically binds CD8, comprises: (i) a VH comprising a VH CDR1, a VH CDR2, and a VH CDR3 having an amino acid sequence of SEQ ID NOs:597, 598, and 599, respectively, and (ii) a VL comprising a VL CDR1, VL CDR2, and VL CDR3 having an amino acid sequence of SEQ ID NOs:600, 601, and 602, respectively. In one embodiment, the first antigen binding domain that specifically binds CD8, comprises: (i) a VH comprising a VH CDR1, a VH CDR2, and a VH CDR3 having an amino acid sequence of SEQ ID NOs:603, 604, and 605, respectively, and (ii) a VL comprising a VL CDR1, VL CDR2, and VL CDR3 having an amino acid sequence of SEQ ID NOs:606, 607, and 608, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:609; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:610. In one embodiment, the first antigen binding domain that specifically binds CD8, comprises a VH having an amino acid sequence of SEQ ID NO:609. In one embodiment, the first antigen binding domain that specifically binds CD8, comprises a VL having an amino acid sequence of SEQ ID NO:610. In one embodiment, the first antigen binding domain that specifically binds CD8, comprises a VH having an amino acid sequence of SEQ ID NO:609, and a VL having an amino acid sequence of SEQ ID NO:610. In one embodiment, the first antigen binding domain that specifically binds CD8, comprises a heavy chain having an amino acid sequence of SEQ ID NO:611. In one embodiment, the first antigen binding domain that specifically binds CD8, comprises a light chain having an amino acid sequence of SEQ ID NO:612. In one embodiment, the first antigen binding domain that specifically binds CD8, comprises a heavy chain having an amino acid sequence of SEQ ID NO:611, and a light chain having an amino acid sequence of SEQ ID NO:612. In one embodiment, the first antigen binding domain that specifically binds CD8, comprises a VH having an amino acid sequence having at least 95% identity to an amino acid sequence of SEQ ID NO:609. In one embodiment, the first antigen binding domain that specifically binds CD8, comprises a VL having an amino acid sequence having at least 95% identity to an amino acid sequence of SEQ ID NO:610. In one embodiment, the first antigen binding domain that specifically binds CD8, comprises a VH having an amino acid sequence having at least 95% identity to an amino acid sequence of SEQ ID NO:609, and a VL having an amino acid sequence having at least 95% identity to an amino acid sequence of SEQ ID NO:610. In one embodiment, the first antigen binding domain that specifically binds CD8, comprises a heavy chain having an amino acid sequence having at least 95% identity to an amino acid sequence of SEQ ID NO:611. In one embodiment, the first antigen binding domain that specifically binds CD8, comprises a light chain having an amino acid sequence having at least 95% identity to an amino acid sequence of SEQ ID NO:612. In one embodiment, the first antigen binding domain that specifically binds CD8, comprises a heavy chain having an amino acid sequence having at least 95% identity to an amino acid sequence of SEQ ID NO:611, and a light chain having an amino acid sequence having at least 95% identity to an amino acid sequence of SEQ ID NO:612.

In one embodiment, the first antigen binding domain that specifically binds CD8, comprises: (i) a VH comprising a VH CDR1, a VH CDR2, and a VH CDR3 having an amino acid sequence of SEQ ID NOs:613, 614, and 615, respectively, and (ii) a VL comprising a VL CDR1, VL CDR2, and VL CDR3 having an amino acid sequence of SEQ ID NOs:616, 617, and 618, respectively. In one embodiment, the first antigen binding domain that specifically binds CD8, comprises: (i) a VH comprising a VH CDR1, a VH CDR2, and a VH CDR3 having an amino acid sequence of SEQ ID NOs:619, 620, and 621, respectively, and (ii) a VL comprising a VL CDR1, VL CDR2, and VL CDR3 having an amino acid sequence of SEQ ID NOs:622, 523, and 624, respectively. In one embodiment, the first antigen binding domain that specifically binds CD8, comprises: (i) a VH comprising a VH CDR1, a VH CDR2, and a VH CDR3 having an amino acid sequence of SEQ ID NOs:625, 626, and 627, respectively, and (ii) a VL comprising a VL CDR1, VL CDR2, and VL CDR3 having an amino acid sequence of SEQ ID NOs:628, 629, and 630, respectively. In one embodiment, the first antigen binding domain that specifically binds CD8, comprises: (i) a VH comprising a VH CDR1, a VH CDR2, and a VH CDR3 having an amino acid sequence of SEQ ID NOs:631, 632, and 633, respectively, and (ii) a VL comprising a VL CDR1, VL CDR2, and VL CDR3 having an amino acid sequence of SEQ ID NOs:634, 635, and 636, respectively. In one embodiment, the first antigen binding domain that specifically binds CD8, comprises: (i) a VH comprising a VH CDR1, a VH CDR2, and a VH CDR3 having an amino acid sequence of SEQ ID NOs:637, 638, and 639, respectively, and (ii) a VL comprising a VL CDR1, VL CDR2, and VL CDR3 having an amino acid sequence of SEQ ID NOs:640, 641, and 642, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:643; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:644. In one embodiment, the first antigen binding domain that specifically binds CD8, comprises a VH having an amino acid sequence of SEQ ID NO:643. In one embodiment, the first antigen binding domain that specifically binds CD8, comprises a VL having an amino acid sequence of SEQ ID NO:644. In one embodiment, the first antigen binding domain that specifically binds CD8, comprises a VH having an amino acid sequence of SEQ ID NO:643, and a VL having an amino acid sequence of SEQ ID NO:644. In one embodiment, the first antigen binding domain that specifically binds CD8, comprises a heavy chain having an amino acid sequence of SEQ ID NO:645. In one embodiment, the first antigen binding domain that specifically binds CD8, comprises a light chain having an amino acid sequence of SEQ ID NO:646. In one embodiment, the first antigen binding domain that specifically binds CD8, comprises a heavy chain having an amino acid sequence of SEQ ID NO:645, and a light chain having an amino acid sequence of SEQ ID NO:646. In one embodiment, the first antigen binding domain that specifically binds CD8, comprises a VH having an amino acid sequence having at least 95% identity to an amino acid sequence of SEQ ID NO:643. In one embodiment, the first antigen binding domain that specifically binds CD8, comprises a VL having an amino acid sequence having at least 95% identity to an amino acid sequence of SEQ ID NO:644. In one embodiment, the first antigen binding domain that specifically binds CD8, comprises a VH having an amino acid sequence having at least 95% identity to an amino acid sequence of SEQ ID NO:643, and a VL having an amino acid sequence having at least 95% identity to an amino acid sequence of SEQ ID NO:644. In one embodiment, the first antigen binding domain that specifically binds CD8, comprises a heavy chain having an amino acid sequence having at least 95% identity to an amino acid sequence of SEQ ID NO:645. In one embodiment, the first antigen binding domain that specifically binds CD8, comprises a light chain having an amino acid sequence having at least 95% identity to an amino acid sequence of SEQ ID NO:646. In one embodiment, the first antigen binding domain that specifically binds CD8, comprises a heavy chain having an amino acid sequence having at least 95% identity to an amino acid sequence of SEQ ID NO:645, and a light chain having an amino acid sequence having at least 95% identity to an amino acid sequence of SEQ ID NO:646.

In one embodiment, the first antigen binding domain that specifically binds CD8, comprises: (i) a VH comprising a VH CDR1, a VH CDR2, and a VH CDR3 having an amino acid sequence of SEQ ID NOs:647, 648, and 649, respectively, and (ii) a VL comprising a VL CDR1, VL CDR2, and VL CDR3 having an amino acid sequence of SEQ ID NOs:650, 651, and 652, respectively. In one embodiment, the first antigen binding domain that specifically binds CD8, comprises: (i) a VH comprising a VH CDR1, a VH CDR2, and a VH CDR3 having an amino acid sequence of SEQ ID NOs:653, 654, and 655, respectively, and (ii) a VL comprising a VL CDR1, VL CDR2, and VL CDR3 having an amino acid sequence of SEQ ID NOs:656, 657, and 658, respectively. In one embodiment, the first antigen binding domain that specifically binds CD8, comprises: (i) a VH comprising a VH CDR1, a VH CDR2, and a VH CDR3 having an amino acid sequence of SEQ ID NOs:659, 660, and 661, respectively, and (ii) a VL comprising a VL CDR1, VL CDR2, and VL CDR3 having an amino acid sequence of SEQ ID NOs:662, 663, and 664, respectively. In one embodiment, the first antigen binding domain that specifically binds CD8, comprises: (i) a VH comprising a VH CDR1, a VH CDR2, and a VH CDR3 having an amino acid sequence of SEQ ID NOs:665, 666, and 667, respectively, and (ii) a VL comprising a VL CDR1, VL CDR2, and VL CDR3 having an amino acid sequence of SEQ ID NOs:668, 669, and 670, respectively. In one embodiment, the first antigen binding domain that specifically binds CD8, comprises: (i) a VH comprising a VH CDR1, a VH CDR2, and a VH CDR3 having an amino acid sequence of SEQ ID NOs:671, 672, and 673, respectively, and (ii) a VL comprising a VL CDR1, VL CDR2, and VL CDR3 having an amino acid sequence of SEQ ID NOs:674, 675, and 676, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:677; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:678. In one embodiment, the first antigen binding domain that specifically binds CD8, comprises a VH having an amino acid sequence of SEQ ID NO:677. In one embodiment, the first antigen binding domain that specifically binds CD8, comprises a VL having an amino acid sequence of SEQ ID NO:678. In one embodiment, the first antigen binding domain that specifically binds CD8, comprises a VH having an amino acid sequence of SEQ ID NO:677, and a VL having an amino acid sequence of SEQ ID NO:678. In one embodiment, the first antigen binding domain that specifically binds CD8, comprises a heavy chain having an amino acid sequence of SEQ ID NO:679. In one embodiment, the first antigen binding domain that specifically binds CD8, comprises a light chain having an amino acid sequence of SEQ ID NO:680. In one embodiment, the first antigen binding domain that specifically binds CD8, comprises a heavy chain having an amino acid sequence of SEQ ID NO:679, and a light chain having an amino acid sequence of SEQ ID NO:680. In one embodiment, the first antigen binding domain that specifically binds CD8, comprises a VH having an amino acid sequence having at least 95% identity to an amino acid sequence of SEQ ID NO:677. In one embodiment, the first antigen binding domain that specifically binds CD8, comprises a VL having an amino acid sequence having at least 95% identity to an amino acid sequence of SEQ ID NO:678. In one embodiment, the first antigen binding domain that specifically binds CD8, comprises a VH having an amino acid sequence having at least 95% identity to an amino acid sequence of SEQ ID NO:677, and a VL having an amino acid sequence having at least 95% identity to an amino acid sequence of SEQ ID NO:678. In one embodiment, the first antigen binding domain that specifically binds CD8, comprises a heavy chain having an amino acid sequence having at least 95% identity to an amino acid sequence of SEQ ID NO:679. In one embodiment, the first antigen binding domain that specifically binds CD8, comprises a light chain having an amino acid sequence having at least 95% identity to an amino acid sequence of SEQ ID NO:680. In one embodiment, the first antigen binding domain that specifically binds CD8, comprises a heavy chain having an amino acid sequence having at least 95% identity to an amino acid sequence of SEQ ID NO:679, and a light chain having an amino acid sequence having at least 95% identity to an amino acid sequence of SEQ ID NO:680.

In one embodiment, the first antigen binding domain that specifically binds CD8, comprises: (i) a VH comprising a VH CDR1, a VH CDR2, and a VH CDR3 having an amino acid sequence of SEQ ID NOs:681, 682, and 683, respectively, and (ii) a VL comprising a VL CDR1, VL CDR2, and VL CDR3 having an amino acid sequence of SEQ ID NOs:684, 685, and 686, respectively. In one embodiment, the first antigen binding domain that specifically binds CD8, comprises: (i) a VH comprising a VH CDR1, a VH CDR2, and a VH CDR3 having an amino acid sequence of SEQ ID NOs:687, 688, and 689, respectively, and (ii) a VL comprising a VL CDR1, VL CDR2, and VL CDR3 having an amino acid sequence of SEQ ID NOs:690, 691, and 692, respectively. In one embodiment, the first antigen binding domain that specifically binds CD8, comprises: (i) a VH comprising a VH CDR1, a VH CDR2, and a VH CDR3 having an amino acid sequence of SEQ ID NOs:693, 694, and 695, respectively, and (ii) a VL comprising a VL CDR1, VL CDR2, and VL CDR3 having an amino acid sequence of SEQ ID NOs:696, 697, and 698, respectively. In one embodiment, the first antigen binding domain that specifically binds CD8, comprises: (i) a VH comprising a VH CDR1, a VH CDR2, and a VH CDR3 having an amino acid sequence of SEQ ID NOs:699, 700, and 701, respectively, and (ii) a VL comprising a VL CDR1, VL CDR2, and VL CDR3 having an amino acid sequence of SEQ ID NOs:702, 703, and 704, respectively. In one embodiment, the first antigen binding domain that specifically binds CD8, comprises: (i) a VH comprising a VH CDR1, a VH CDR2, and a VH CDR3 having an amino acid sequence of SEQ ID NOs:705, 706, and 707, respectively, and (ii) a VL comprising a VL CDR1, VL CDR2, and VL CDR3 having an amino acid sequence of SEQ ID NOs:708, 709, and 710, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:711; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:712. In one embodiment, the first antigen binding domain that specifically binds CD8, comprises a VH having an amino acid sequence of SEQ ID NO:711. In one embodiment, the first antigen binding domain that specifically binds CD8, comprises a VL having an amino acid sequence of SEQ ID NO:712. In one embodiment, the first antigen binding domain that specifically binds CD8, comprises a VH having an amino acid sequence of SEQ ID NO:711, and a VL having an amino acid sequence of SEQ ID NO:712. In one embodiment, the first antigen binding domain that specifically binds CD8, comprises a heavy chain having an amino acid sequence of SEQ ID NO:713. In one embodiment, the first antigen binding domain that specifically binds CD8, comprises a light chain having an amino acid sequence of SEQ ID NO:714. In one embodiment, the first antigen binding domain that specifically binds CD8, comprises a heavy chain having an amino acid sequence of SEQ ID NO:713, and a light chain having an amino acid sequence of SEQ ID NO:714. In one embodiment, the first antigen binding domain that specifically binds CD8, comprises a VH having an amino acid sequence having at least 95% identity to an amino acid sequence of SEQ ID NO:711. In one embodiment, the first antigen binding domain that specifically binds CD8, comprises a VL having an amino acid sequence having at least 95% identity to an amino acid sequence of SEQ ID NO:712. In one embodiment, the first antigen binding domain that specifically binds CD8, comprises a VH having an amino acid sequence having at least 95% identity to an amino acid sequence of SEQ ID NO:711, and a VL having an amino acid sequence having at least 95% identity to an amino acid sequence of SEQ ID NO:712. In one embodiment, the first antigen binding domain that specifically binds CD8, comprises a heavy chain having an amino acid sequence having at least 95% identity to an amino acid sequence of SEQ ID NO:713. In one embodiment, the first antigen binding domain that specifically binds CD8, comprises a light chain having an amino acid sequence having at least 95% identity to an amino acid sequence of SEQ ID NO:714. In one embodiment, the first antigen binding domain that specifically binds CD8, comprises a heavy chain having an amino acid sequence having at least 95% identity to an amino acid sequence of SEQ ID NO:713, and a light chain having an amino acid sequence having at least 95% identity to an amino acid sequence of SEQ ID NO:714.

In one embodiment, the first antigen binding domain that specifically binds CD8, comprises: (i) a VH comprising a VH CDR1, a VH CDR2, and a VH CDR3 having an amino acid sequence of SEQ ID NOs:715, 716, and 717, respectively, and (ii) a VL comprising a VL CDR1, VL CDR2, and VL CDR3 having an amino acid sequence of SEQ ID NOs:718, 719, and 720, respectively. In one embodiment, the first antigen binding domain that specifically binds CD8, comprises: (i) a VH comprising a VH CDR1, a VH CDR2, and a VH CDR3 having an amino acid sequence of SEQ ID NOs:721, 722, and 723, respectively, and (ii) a VL comprising a VL CDR1, VL CDR2, and VL CDR3 having an amino acid sequence of SEQ ID NOs:724, 725, and 726, respectively. In one embodiment, the first antigen binding domain that specifically binds CD8, comprises: (i) a VH comprising a VH CDR1, a VH CDR2, and a VH CDR3 having an amino acid sequence of SEQ ID NOs:727, 728, and 729, respectively, and (ii) a VL comprising a VL CDR1, VL CDR2, and VL CDR3 having an amino acid sequence of SEQ ID NOs:730, 731, and 732, respectively. In one embodiment, the first antigen binding domain that specifically binds CD8, comprises: (i) a VH comprising a VH CDR1, a VH CDR2, and a VH CDR3 having an amino acid sequence of SEQ ID NOs:733, 734, and 735, respectively, and (ii) a VL comprising a VL CDR1, VL CDR2, and VL CDR3 having an amino acid sequence of SEQ ID NOs:736, 737, and 738, respectively. In one embodiment, the first antigen binding domain that specifically binds CD8, comprises: (i) a VH comprising a VH CDR1, a VH CDR2, and a VH CDR3 having an amino acid sequence of SEQ ID NOs:739, 740, and 741, respectively, and (ii) a VL comprising a VL CDR1, VL CDR2, and VL CDR3 having an amino acid sequence of SEQ ID NOs:742, 743, and 744, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:745; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:746. In one embodiment, the first antigen binding domain that specifically binds CD8, comprises a VH having an amino acid sequence of SEQ ID NO:745. In one embodiment, the first antigen binding domain that specifically binds CD8, comprises a VL having an amino acid sequence of SEQ ID NO:746. In one embodiment, the first antigen binding domain that specifically binds CD8, comprises a VH having an amino acid sequence of SEQ ID NO:745, and a VL having an amino acid sequence of SEQ ID NO:746. In one embodiment, the first antigen binding domain that specifically binds CD8, comprises a heavy chain having an amino acid sequence of SEQ ID NO:747. In one embodiment, the first antigen binding domain that specifically binds CD8, comprises a light chain having an amino acid sequence of SEQ ID NO:748. In one embodiment, the first antigen binding domain that specifically binds CD8, comprises a heavy chain having an amino acid sequence of SEQ ID NO:747, and a light chain having an amino acid sequence of SEQ ID NO:748. In one embodiment, the first antigen binding domain that specifically binds CD8, comprises a VH having an amino acid sequence having at least 95% identity to an amino acid sequence of SEQ ID NO:745. In one embodiment, the first antigen binding domain that specifically binds CD8, comprises a VL having an amino acid sequence having at least 95% identity to an amino acid sequence of SEQ ID NO:746. In one embodiment, the first antigen binding domain that specifically binds CD8, comprises a VH having an amino acid sequence having at least 95% identity to an amino acid sequence of SEQ ID NO:745, and a VL having an amino acid sequence having at least 95% identity to an amino acid sequence of SEQ ID NO:746. In one embodiment, the first antigen binding domain that specifically binds CD8, comprises a heavy chain having an amino acid sequence having at least 95% identity to an amino acid sequence of SEQ ID NO:747. In one embodiment, the first antigen binding domain that specifically binds CD8, comprises a light chain having an amino acid sequence having at least 95% identity to an amino acid sequence of SEQ ID NO:748. In one embodiment, the first antigen binding domain that specifically binds CD8, comprises a heavy chain having an amino acid sequence having at least 95% identity to an amino acid sequence of SEQ ID NO:747, and a light chain having an amino acid sequence having at least 95% identity to an amino acid sequence of SEQ ID NO:748.

In one embodiment, the first antigen binding domain that specifically binds CD8, comprises: (i) a VH comprising a VH CDR1, a VH CDR2, and a VH CDR3 having an amino acid sequence of SEQ ID NOs:749, 750, and 751, respectively, and (ii) a VL comprising a VL CDR1, VL CDR2, and VL CDR3 having an amino acid sequence of SEQ ID NOs:752, 753, and 754, respectively. In one embodiment, the first antigen binding domain that specifically binds CD8, comprises: (i) a VH comprising a VH CDR1, a VH CDR2, and a VH CDR3 having an amino acid sequence of SEQ ID NOs:755, 756, and 757, respectively, and (ii) a VL comprising a VL CDR1, VL CDR2, and VL CDR3 having an amino acid sequence of SEQ ID NOs:758, 759, and 760, respectively. In one embodiment, the first antigen binding domain that specifically binds CD8, comprises: (i) a VH comprising a VH CDR1, a VH CDR2, and a VH CDR3 having an amino acid sequence of SEQ ID NOs:761, 762, and 763, respectively, and (ii) a VL comprising a VL CDR1, VL CDR2, and VL CDR3 having an amino acid sequence of SEQ ID NOs:764, 765, and 766, respectively. In one embodiment, the first antigen binding domain that specifically binds CD8, comprises: (i) a VH comprising a VH CDR1, a VH CDR2, and a VH CDR3 having an amino acid sequence of SEQ ID NOs:767, 768, and 769, respectively, and (ii) a VL comprising a VL CDR1, VL CDR2, and VL CDR3 having an amino acid sequence of SEQ ID NOs:770, 771, and 772, respectively. In one embodiment, the first antigen binding domain that specifically binds CD8, comprises: (i) a VH comprising a VH CDR1, a VH CDR2, and a VH CDR3 having an amino acid sequence of SEQ ID NOs:773, 774, and 775, respectively, and (ii) a VL comprising a VL CDR1, VL CDR2, and VL CDR3 having an amino acid sequence of SEQ ID NOs:776, 777, and 778, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:779; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:780. In one embodiment, the first antigen binding domain that specifically binds CD8, comprises a VH having an amino acid sequence of SEQ ID NO:779. In one embodiment, the first antigen binding domain that specifically binds CD8, comprises a VL having an amino acid sequence of SEQ ID NO:780. In one embodiment, the first antigen binding domain that specifically binds CD8, comprises a VH having an amino acid sequence of SEQ ID NO:779, and a VL having an amino acid sequence of SEQ ID NO:780. In one embodiment, the first antigen binding domain that specifically binds CD8, comprises a heavy chain having an amino acid sequence of SEQ ID NO:781. In one embodiment, the first antigen binding domain that specifically binds CD8, comprises a light chain having an amino acid sequence of SEQ ID NO:782. In one embodiment, the first antigen binding domain that specifically binds CD8, comprises a heavy chain having an amino acid sequence of SEQ ID NO:781, and a light chain having an amino acid sequence of SEQ ID NO:782. In one embodiment, the first antigen binding domain that specifically binds CD8, comprises a VH having an amino acid sequence having at least 95% identity to an amino acid sequence of SEQ ID NO:779. In one embodiment, the first antigen binding domain that specifically binds CD8, comprises a VL having an amino acid sequence having at least 95% identity to an amino acid sequence of SEQ ID NO:780. In one embodiment, the first antigen binding domain that specifically binds CD8, comprises a VH having an amino acid sequence having at least 95% identity to an amino acid sequence of SEQ ID NO:779, and a VL having an amino acid sequence having at least 95% identity to an amino acid sequence of SEQ ID NO:780. In one embodiment, the first antigen binding domain that specifically binds CD8, comprises a heavy chain having an amino acid sequence having at least 95% identity to an amino acid sequence of SEQ ID NO:781. In one embodiment, the first antigen binding domain that specifically binds CD8, comprises a light chain having an amino acid sequence having at least 95% identity to an amino acid sequence of SEQ ID NO:782. In one embodiment, the first antigen binding domain that specifically binds CD8, comprises a heavy chain having an amino acid sequence having at least 95% identity to an amino acid sequence of SEQ ID NO:781, and a light chain having an amino acid sequence having at least 95% identity to an amino acid sequence of SEQ ID NO:782.

In one embodiment, the first antigen binding domain that specifically binds CD8, comprises: (i) a VH comprising a VH CDR1, a VH CDR2, and a VH CDR3 having an amino acid sequence of SEQ ID NOs:783, 784, and 785, respectively, and (ii) a VL comprising a VL CDR1, VL CDR2, and VL CDR3 having an amino acid sequence of SEQ ID NOs:786, 787, and 788, respectively. In one embodiment, the first antigen binding domain that specifically binds CD8, comprises: (i) a VH comprising a VH CDR1, a VH CDR2, and a VH CDR3 having an amino acid sequence of SEQ ID NOs:789, 790, and 791, respectively, and (ii) a VL comprising a VL CDR1, VL CDR2, and VL CDR3 having an amino acid sequence of SEQ ID NOs:792, 793, and 794, respectively. In one embodiment, the first antigen binding domain that specifically binds CD8, comprises: (i) a VH comprising a VH CDR1, a VH CDR2, and a VH CDR3 having an amino acid sequence of SEQ ID NOs:795, 796, and 797, respectively, and (ii) a VL comprising a VL CDR1, VL CDR2, and VL CDR3 having an amino acid sequence of SEQ ID NOs:798, 799, and 800, respectively. In one embodiment, the first antigen binding domain that specifically binds CD8, comprises: (i) a VH comprising a VH CDR1, a VH CDR2, and a VH CDR3 having an amino acid sequence of SEQ ID NOs:801, 802, and 803, respectively, and (ii) a VL comprising a VL CDR1, VL CDR2, and VL CDR3 having an amino acid sequence of SEQ ID NOs:804, 805, and 806, respectively. In one embodiment, the first antigen binding domain that specifically binds CD8, comprises: (i) a VH comprising a VH CDR1, a VH CDR2, and a VH CDR3 having an amino acid sequence of SEQ ID NOs:807, 808, and 809, respectively, and (ii) a VL comprising a VL CDR1, VL CDR2, and VL CDR3 having an amino acid sequence of SEQ ID NOs:810, 811, and 812, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:813; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:814. In one embodiment, the first antigen binding domain that specifically binds CD8, comprises a VH having an amino acid sequence of SEQ ID NO:813. In one embodiment, the first antigen binding domain that specifically binds CD8, comprises a VL having an amino acid sequence of SEQ ID NO:814. In one embodiment, the first antigen binding domain that specifically binds CD8, comprises a VH having an amino acid sequence of SEQ ID NO:813, and a VL having an amino acid sequence of SEQ ID NO:814. In one embodiment, the first antigen binding domain that specifically binds CD8, comprises a heavy chain having an amino acid sequence of SEQ ID NO:815. In one embodiment, the first antigen binding domain that specifically binds CD8, comprises a light chain having an amino acid sequence of SEQ ID NO:816. In one embodiment, the first antigen binding domain that specifically binds CD8, comprises a heavy chain having an amino acid sequence of SEQ ID NO:815, and a light chain having an amino acid sequence of SEQ ID NO:816. In one embodiment, the first antigen binding domain that specifically binds CD8, comprises a VH having an amino acid sequence having at least 95% identity to an amino acid sequence of SEQ ID NO:813. In one embodiment, the first antigen binding domain that specifically binds CD8, comprises a VL having an amino acid sequence having at least 95% identity to an amino acid sequence of SEQ ID NO:814. In one embodiment, the first antigen binding domain that specifically binds CD8, comprises a VH having an amino acid sequence having at least 95% identity to an amino acid sequence of SEQ ID NO:813, and a VL having an amino acid sequence having at least 95% identity to an amino acid sequence of SEQ ID NO:814. In one embodiment, the first antigen binding domain that specifically binds CD8, comprises a heavy chain having an amino acid sequence having at least 95% identity to an amino acid sequence of SEQ ID NO:815. In one embodiment, the first antigen binding domain that specifically binds CD8, comprises a light chain having an amino acid sequence having at least 95% identity to an amino acid sequence of SEQ ID NO:816. In one embodiment, the first antigen binding domain that specifically binds CD8, comprises a heavy chain having an amino acid sequence having at least 95% identity to an amino acid sequence of SEQ ID NO:815, and a light chain having an amino acid sequence having at least 95% identity to an amino acid sequence of SEQ ID NO:816.

In one embodiment, the first antigen binding domain that specifically binds CD8, comprises: (i) a VH comprising a VH CDR1, a VH CDR2, and a VH CDR3 having an amino acid sequence of SEQ ID NOs:817, 818, and 819, respectively, and (ii) a VL comprising a VL CDR1, VL CDR2, and VL CDR3 having an amino acid sequence of SEQ ID NOs:820, 821, and 822, respectively. In one embodiment, the first antigen binding domain that specifically binds CD8, comprises: (i) a VH comprising a VH CDR1, a VH CDR2, and a VH CDR3 having an amino acid sequence of SEQ ID NOs:823, 824, and 825, respectively, and (ii) a VL comprising a VL CDR1, VL CDR2, and VL CDR3 having an amino acid sequence of SEQ ID NOs:826, 827, and 828, respectively. In one embodiment, the first antigen binding domain that specifically binds CD8, comprises: (i) a VH comprising a VH CDR1, a VH CDR2, and a VH CDR3 having an amino acid sequence of SEQ ID NOs:829, 830, and 831, respectively, and (ii) a VL comprising a VL CDR1, VL CDR2, and VL CDR3 having an amino acid sequence of SEQ ID NOs:832, 833, and 834, respectively. In one embodiment, the first antigen binding domain that specifically binds CD8, comprises: (i) a VH comprising a VH CDR1, a VH CDR2, and a VH CDR3 having an amino acid sequence of SEQ ID NOs:835, 836, and 837, respectively, and (ii) a VL comprising a VL CDR1, VL CDR2, and VL CDR3 having an amino acid sequence of SEQ ID NOs:838, 839, and 840, respectively. In one embodiment, the first antigen binding domain that specifically binds CD8, comprises: (i) a VH comprising a VH CDR1, a VH CDR2, and a VH CDR3 having an amino acid sequence of SEQ ID NOs:841, 842, and 843, respectively, and (ii) a VL comprising a VL CDR1, VL CDR2, and VL CDR3 having an amino acid sequence of SEQ ID NOs:844, 845, and 846, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:847; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:848. In one embodiment, the first antigen binding domain that specifically binds CD8, comprises a VH having an amino acid sequence of SEQ ID NO:847. In one embodiment, the first antigen binding domain that specifically binds CD8, comprises a VL having an amino acid sequence of SEQ ID NO:848. In one embodiment, the first antigen binding domain that specifically binds CD8, comprises a VH having an amino acid sequence of SEQ ID NO:847, and a VL having an amino acid sequence of SEQ ID NO:848. In one embodiment, the first antigen binding domain that specifically binds CD8, comprises a heavy chain having an amino acid sequence of SEQ ID NO:849. In one embodiment, the first antigen binding domain that specifically binds CD8, comprises a light chain having an amino acid sequence of SEQ ID NO:850. In one embodiment, the first antigen binding domain that specifically binds CD8, comprises a heavy chain having an amino acid sequence of SEQ ID NO:849, and a light chain having an amino acid sequence of SEQ ID NO:850. In one embodiment, the first antigen binding domain that specifically binds CD8, comprises a VH having an amino acid sequence having at least 95% identity to an amino acid sequence of SEQ ID NO:847. In one embodiment, the first antigen binding domain that specifically binds CD8, comprises a VL having an amino acid sequence having at least 95% identity to an amino acid sequence of SEQ ID NO:848. In one embodiment, the first antigen binding domain that specifically binds CD8, comprises a VH having an amino acid sequence having at least 95% identity to an amino acid sequence of SEQ ID NO:847, and a VL having an amino acid sequence having at least 95% identity to an amino acid sequence of SEQ ID NO:848. In one embodiment, the first antigen binding domain that specifically binds CD8, comprises a heavy chain having an amino acid sequence having at least 95% identity to an amino acid sequence of SEQ ID NO:849. In one embodiment, the first antigen binding domain that specifically binds CD8, comprises a light chain having an amino acid sequence having at least 95% identity to an amino acid sequence of SEQ ID NO:850. In one embodiment, the first antigen binding domain that specifically binds CD8, comprises a heavy chain having an amino acid sequence having at least 95% identity to an amino acid sequence of SEQ ID NO:849, and a light chain having an amino acid sequence having at least 95% identity to an amino acid sequence of SEQ ID NO:850.

In one embodiment, the first antigen binding domain that specifically binds CD8, comprises: (i) a VH comprising a VH CDR1, a VH CDR2, and a VH CDR3 having an amino acid sequence of SEQ ID NOs:851, 852, and 853, respectively, and (ii) a VL comprising a VL CDR1, VL CDR2, and VL CDR3 having an amino acid sequence of SEQ ID NOs:854, 855, and 856, respectively. In one embodiment, the first antigen binding domain that specifically binds CD8, comprises: (i) a VH comprising a VH CDR1, a VH CDR2, and a VH CDR3 having an amino acid sequence of SEQ ID NOs:857, 858, and 859, respectively, and (ii) a VL comprising a VL CDR1, VL CDR2, and VL CDR3 having an amino acid sequence of SEQ ID NOs:860, 861, and 862, respectively. In one embodiment, the first antigen binding domain that specifically binds CD8, comprises: (i) a VH comprising a VH CDR1, a VH CDR2, and a VH CDR3 having an amino acid sequence of SEQ ID NOs:863, 864, and 865, respectively, and (ii) a VL comprising a VL CDR1, VL CDR2, and VL CDR3 having an amino acid sequence of SEQ ID NOs:866, 867, and 868, respectively. In one embodiment, the first antigen binding domain that specifically binds CD8, comprises: (i) a VH comprising a VH CDR1, a VH CDR2, and a VH CDR3 having an amino acid sequence of SEQ ID NOs:869, 870, and 871, respectively, and (ii) a VL comprising a VL CDR1, VL CDR2, and VL CDR3 having an amino acid sequence of SEQ ID NOs:872, 873, and 874, respectively. In one embodiment, the first antigen binding domain that specifically binds CD8, comprises: (i) a VH comprising a VH CDR1, a VH CDR2, and a VH CDR3 having an amino acid sequence of SEQ ID NOs:875, 876, and 877, respectively, and (ii) a VL comprising a VL CDR1, VL CDR2, and VL CDR3 having an amino acid sequence of SEQ ID NOs:878, 879, and 880, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:881; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:882. In one embodiment, the first antigen binding domain that specifically binds CD8, comprises a VH having an amino acid sequence of SEQ ID NO:881. In one embodiment, the first antigen binding domain that specifically binds CD8, comprises a VL having an amino acid sequence of SEQ ID NO:882. In one embodiment, the first antigen binding domain that specifically binds CD8, comprises a VH having an amino acid sequence of SEQ ID NO:881, and a VL having an amino acid sequence of SEQ ID NO:882. In one embodiment, the first antigen binding domain that specifically binds CD8, comprises a heavy chain having an amino acid sequence of SEQ ID NO:883. In one embodiment, the first antigen binding domain that specifically binds CD8, comprises a light chain having an amino acid sequence of SEQ ID NO:884. In one embodiment, the first antigen binding domain that specifically binds CD8, comprises a heavy chain having an amino acid sequence of SEQ ID NO:883, and a light chain having an amino acid sequence of SEQ ID NO:884. In one embodiment, the first antigen binding domain that specifically binds CD8, comprises a VH having an amino acid sequence having at least 95% identity to an amino acid sequence of SEQ ID NO:881. In one embodiment, the first antigen binding domain that specifically binds CD8, comprises a VL having an amino acid sequence having at least 95% identity to an amino acid sequence of SEQ ID NO:882. In one embodiment, the first antigen binding domain that specifically binds CD8, comprises a VH having an amino acid sequence having at least 95% identity to an amino acid sequence of SEQ ID NO:881, and a VL having an amino acid sequence having at least 95% identity to an amino acid sequence of SEQ ID NO:882. In one embodiment, the first antigen binding domain that specifically binds CD8, comprises a heavy chain having an amino acid sequence having at least 95% identity to an amino acid sequence of SEQ ID NO:883. In one embodiment, the first antigen binding domain that specifically binds CD8, comprises a light chain having an amino acid sequence having at least 95% identity to an amino acid sequence of SEQ ID NO:884. In one embodiment, the first antigen binding domain that specifically binds CD8, comprises a heavy chain having an amino acid sequence having at least 95% identity to an amino acid sequence of SEQ ID NO:883, and a light chain having an amino acid sequence having at least 95% identity to an amino acid sequence of SEQ ID NO:884.

In one embodiment, the first antigen binding domain that specifically binds CD8, comprises: (i) a VH comprising a VH CDR1, a VH CDR2, and a VH CDR3 having an amino acid sequence of SEQ ID NOs:885, 886, and 887, respectively, and (ii) a VL comprising a VL CDR1, VL CDR2, and VL CDR3 having an amino acid sequence of SEQ ID NOs:888, 889, and 890, respectively. In one embodiment, the first antigen binding domain that specifically binds CD8, comprises: (i) a VH comprising a VH CDR1, a VH CDR2, and a VH CDR3 having an amino acid sequence of SEQ ID NOs:891, 892, and 893, respectively, and (ii) a VL comprising a VL CDR1, VL CDR2, and VL CDR3 having an amino acid sequence of SEQ ID NOs:894, 895, and 896, respectively. In one embodiment, the first antigen binding domain that specifically binds CD8, comprises: (i) a VH comprising a VH CDR1, a VH CDR2, and a VH CDR3 having an amino acid sequence of SEQ ID NOs:897, 898, and 899, respectively, and (ii) a VL comprising a VL CDR1, VL CDR2, and VL CDR3 having an amino acid sequence of SEQ ID NOs:900, 901, and 902, respectively. In one embodiment, the first antigen binding domain that specifically binds CD8, comprises: (i) a VH comprising a VH CDR1, a VH CDR2, and a VH CDR3 having an amino acid sequence of SEQ ID NOs:903, 904, and 905, respectively, and (ii) a VL comprising a VL CDR1, VL CDR2, and VL CDR3 having an amino acid sequence of SEQ ID NOs:906, 907, and 908, respectively. In one embodiment, the first antigen binding domain that specifically binds CD8, comprises: (i) a VH comprising a VH CDR1, a VH CDR2, and a VH CDR3 having an amino acid sequence of SEQ ID NOs:909, 910, and 911, respectively, and (ii) a VL comprising a VL CDR1, VL CDR2, and VL CDR3 having an amino acid sequence of SEQ ID NOs:912, 913, and 914, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:915; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:916. In one embodiment, the first antigen binding domain that specifically binds CD8, comprises a VH having an amino acid sequence of SEQ ID NO:915. In one embodiment, the first antigen binding domain that specifically binds CD8, comprises a VL having an amino acid sequence of SEQ ID NO:916. In one embodiment, the first antigen binding domain that specifically binds CD8, comprises a VH having an amino acid sequence of SEQ ID NO:915, and a VL having an amino acid sequence of SEQ ID NO:916. In one embodiment, the first antigen binding domain that specifically binds CD8, comprises a heavy chain having an amino acid sequence of SEQ ID NO:917. In one embodiment, the first antigen binding domain that specifically binds CD8, comprises a light chain having an amino acid sequence of SEQ ID NO:918. In one embodiment, the first antigen binding domain that specifically binds CD8, comprises a heavy chain having an amino acid sequence of SEQ ID NO:917, and a light chain having an amino acid sequence of SEQ ID NO:918. In one embodiment, the first antigen binding domain that specifically binds CD8, comprises a VH having an amino acid sequence having at least 95% identity to an amino acid sequence of SEQ ID NO:915. In one embodiment, the first antigen binding domain that specifically binds CD8, comprises a VL having an amino acid sequence having at least 95% identity to an amino acid sequence of SEQ ID NO:916. In one embodiment, the first antigen binding domain that specifically binds CD8, comprises a VH having an amino acid sequence having at least 95% identity to an amino acid sequence of SEQ ID NO:915, and a VL having an amino acid sequence having at least 95% identity to an amino acid sequence of SEQ ID NO:916. In one embodiment, the first antigen binding domain that specifically binds CD8, comprises a heavy chain having an amino acid sequence having at least 95% identity to an amino acid sequence of SEQ ID NO:917. In one embodiment, the first antigen binding domain that specifically binds CD8, comprises a light chain having an amino acid sequence having at least 95% identity to an amino acid sequence of SEQ ID NO:918. In one embodiment, the first antigen binding domain that specifically binds CD8, comprises a heavy chain having an amino acid sequence having at least 95% identity to an amino acid sequence of SEQ ID NO:917, and a light chain having an amino acid sequence having at least 95% identity to an amino acid sequence of SEQ ID NO:918.

In one embodiment, the first antigen binding domain that specifically binds CD8, comprises: (i) a VH comprising a VH CDR1, a VH CDR2, and a VH CDR3 having an amino acid sequence of SEQ ID NOs:919, 920, and 921, respectively, and (ii) a VL comprising a VL CDR1, VL CDR2, and VL CDR3 having an amino acid sequence of SEQ ID NOs:922, 923, and 924, respectively. In one embodiment, the first antigen binding domain that specifically binds CD8, comprises: (i) a VH comprising a VH CDR1, a VH CDR2, and a VH CDR3 having an amino acid sequence of SEQ ID NOs:925, 926, and 927, respectively, and (ii) a VL comprising a VL CDR1, VL CDR2, and VL CDR3 having an amino acid sequence of SEQ ID NOs:928, 929, and 930, respectively. In one embodiment, the first antigen binding domain that specifically binds CD8, comprises: (i) a VH comprising a VH CDR1, a VH CDR2, and a VH CDR3 having an amino acid sequence of SEQ ID NOs:931, 932, and 933, respectively, and (ii) a VL comprising a VL CDR1, VL CDR2, and VL CDR3 having an amino acid sequence of SEQ ID NOs:934, 935, and 936, respectively. In one embodiment, the first antigen binding domain that specifically binds CD8, comprises: (i) a VH comprising a VH CDR1, a VH CDR2, and a VH CDR3 having an amino acid sequence of SEQ ID NOs:937, 938, and 939, respectively, and (ii) a VL comprising a VL CDR1, VL CDR2, and VL CDR3 having an amino acid sequence of SEQ ID NOs:940, 941, and 942, respectively. In one embodiment, the first antigen binding domain that specifically binds CD8, comprises: (i) a VH comprising a VH CDR1, a VH CDR2, and a VH CDR3 having an amino acid sequence of SEQ ID NOs:943, 944, and 945, respectively, and (ii) a VL comprising a VL CDR1, VL CDR2, and VL CDR3 having an amino acid sequence of SEQ ID NOs:946, 947, and 948, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:949; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:950. In one embodiment, the first antigen binding domain that specifically binds CD8, comprises a VH having an amino acid sequence of SEQ ID NO:949. In one embodiment, the first antigen binding domain that specifically binds CD8, comprises a VL having an amino acid sequence of SEQ ID NO:950. In one embodiment, the first antigen binding domain that specifically binds CD8, comprises a VH having an amino acid sequence of SEQ ID NO:949, and a VL having an amino acid sequence of SEQ ID NO:950. In one embodiment, the first antigen binding domain that specifically binds CD8, comprises a heavy chain having an amino acid sequence of SEQ ID NO:951. In one embodiment, the first antigen binding domain that specifically binds CD8, comprises a light chain having an amino acid sequence of SEQ ID NO:952. In one embodiment, the first antigen binding domain that specifically binds CD8, comprises a heavy chain having an amino acid sequence of SEQ ID NO:951, and a light chain having an amino acid sequence of SEQ ID NO:952. In one embodiment, the first antigen binding domain that specifically binds CD8, comprises a VH having an amino acid sequence having at least 95% identity to an amino acid sequence of SEQ ID NO:949. In one embodiment, the first antigen binding domain that specifically binds CD8, comprises a VL having an amino acid sequence having at least 95% identity to an amino acid sequence of SEQ ID NO:950. In one embodiment, the first antigen binding domain that specifically binds CD8, comprises a VH having an amino acid sequence having at least 95% identity to an amino acid sequence of SEQ ID NO:949, and a VL having an amino acid sequence having at least 95% identity to an amino acid sequence of SEQ ID NO:950. In one embodiment, the first antigen binding domain that specifically binds CD8, comprises a heavy chain having an amino acid sequence having at least 95% identity to an amino acid sequence of SEQ ID NO:951. In one embodiment, the first antigen binding domain that specifically binds CD8, comprises a light chain having an amino acid sequence having at least 95% identity to an amino acid sequence of SEQ ID NO:952. In one embodiment, the first antigen binding domain that specifically binds CD8, comprises a heavy chain having an amino acid sequence having at least 95% identity to an amino acid sequence of SEQ ID NO:951, and a light chain having an amino acid sequence having at least 95% identity to an amino acid sequence of SEQ ID NO:952.

In one embodiment, the first antigen binding domain that specifically binds CD8, comprises: (i) a VH comprising a VH CDR1, a VH CDR2, and a VH CDR3 having an amino acid sequence of SEQ ID NOs:953, 954, and 955, respectively, and (ii) a VL comprising a VL CDR1, VL CDR2, and VL CDR3 having an amino acid sequence of SEQ ID NOs:956, 957, and 958, respectively. In one embodiment, the first antigen binding domain that specifically binds CD8, comprises: (i) a VH comprising a VH CDR1, a VH CDR2, and a VH CDR3 having an amino acid sequence of SEQ ID NOs:959, 960, and 961, respectively, and (ii) a VL comprising a VL CDR1, VL CDR2, and VL CDR3 having an amino acid sequence of SEQ ID NOs:962, 963, and 964, respectively. In one embodiment, the first antigen binding domain that specifically binds CD8, comprises: (i) a VH comprising a VH CDR1, a VH CDR2, and a VH CDR3 having an amino acid sequence of SEQ ID NOs:965, 966, and 967, respectively, and (ii) a VL comprising a VL CDR1, VL CDR2, and VL CDR3 having an amino acid sequence of SEQ ID NOs:968, 969, and 970, respectively. In one embodiment, the first antigen binding domain that specifically binds CD8, comprises: (i) a VH comprising a VH CDR1, a VH CDR2, and a VH CDR3 having an amino acid sequence of SEQ ID NOs:971, 972, and 973, respectively, and (ii) a VL comprising a VL CDR1, VL CDR2, and VL CDR3 having an amino acid sequence of SEQ ID NOs:974, 975, and 976, respectively. In one embodiment, the first antigen binding domain that specifically binds CD8, comprises: (i) a VH comprising a VH CDR1, a VH CDR2, and a VH CDR3 having an amino acid sequence of SEQ ID NOs:977, 978, and 979, respectively, and (ii) a VL comprising a VL CDR1, VL CDR2, and VL CDR3 having an amino acid sequence of SEQ ID NOs:980, 981, and 982, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:983; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:984. In one embodiment, the first antigen binding domain that specifically binds CD8, comprises a VH having an amino acid sequence of SEQ ID NO:983. In one embodiment, the first antigen binding domain that specifically binds CD8, comprises a VL having an amino acid sequence of SEQ ID NO:984. In one embodiment, the first antigen binding domain that specifically binds CD8, comprises a VH having an amino acid sequence of SEQ ID NO:983, and a VL having an amino acid sequence of SEQ ID NO:984. In one embodiment, the first antigen binding domain that specifically binds CD8, comprises a heavy chain having an amino acid sequence of SEQ ID NO:985. In one embodiment, the first antigen binding domain that specifically binds CD8, comprises a light chain having an amino acid sequence of SEQ ID NO:986. In one embodiment, the first antigen binding domain that specifically binds CD8, comprises a heavy chain having an amino acid sequence of SEQ ID NO:985, and a light chain having an amino acid sequence of SEQ ID NO:986. In one embodiment, the first antigen binding domain that specifically binds CD8, comprises a VH having an amino acid sequence having at least 95% identity to an amino acid sequence of SEQ ID NO:983. In one embodiment, the first antigen binding domain that specifically binds CD8, comprises a VL having an amino acid sequence having at least 95% identity to an amino acid sequence of SEQ ID NO:984. In one embodiment, the first antigen binding domain that specifically binds CD8, comprises a VH having an amino acid sequence having at least 95% identity to an amino acid sequence of SEQ ID NO:983, and a VL having an amino acid sequence having at least 95% identity to an amino acid sequence of SEQ ID NO:984. In one embodiment, the first antigen binding domain that specifically binds CD8, comprises a heavy chain having an amino acid sequence having at least 95% identity to an amino acid sequence of SEQ ID NO:985. In one embodiment, the first antigen binding domain that specifically binds CD8, comprises a light chain having an amino acid sequence having at least 95% identity to an amino acid sequence of SEQ ID NO:986. In one embodiment, the first antigen binding domain that specifically binds CD8, comprises a heavy chain having an amino acid sequence having at least 95% identity to an amino acid sequence of SEQ ID NO:985, and a light chain having an amino acid sequence having at least 95% identity to an amino acid sequence of SEQ ID NO:986.

In one embodiment, the first antigen binding domain that specifically binds CD8, comprises: (i) a VH comprising a VH CDR1, a VH CDR2, and a VH CDR3 having an amino acid sequence of SEQ ID NOs:987, 988, and 989, respectively, and (ii) a VL comprising a VL CDR1, VL CDR2, and VL CDR3 having an amino acid sequence of SEQ ID NOs:990, 991, and 992, respectively. In one embodiment, the first antigen binding domain that specifically binds CD8, comprises: (i) a VH comprising a VH CDR1, a VH CDR2, and a VH CDR3 having an amino acid sequence of SEQ ID NOs:993, 994, and 995, respectively, and (ii) a VL comprising a VL CDR1, VL CDR2, and VL CDR3 having an amino acid sequence of SEQ ID NOs:996, 997, and 998, respectively. In one embodiment, the first antigen binding domain that specifically binds CD8, comprises: (i) a VH comprising a VH CDR1, a VH CDR2, and a VH CDR3 having an amino acid sequence of SEQ ID NOs:999, 1000, and 1001, respectively, and (ii) a VL comprising a VL CDR1, VL CDR2, and VL CDR3 having an amino acid sequence of SEQ ID NOs:1002, 1003, and 1004, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1005, 1006, and 1007, respectively, and (ii) a VL comprising a VL CDR1, VL CDR2, and VL CDR3 having an amino acid sequence of SEQ ID NOs:1008, 1009, and 1010, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1011, 1012, and 1013, respectively, and (ii) a VL comprising a VL CDR1, VL CDR2, and VL CDR3 having an amino acid sequence of SEQ ID NOs:1014, 1015, and 1016, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1017; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1018. In one embodiment, the first antigen binding domain that specifically binds CD8, comprises a VH having an amino acid sequence of SEQ ID NO:1017. In one embodiment, the first antigen binding domain that specifically binds CD8, comprises a VL having an amino acid sequence of SEQ ID NO:1018. In one embodiment, the first antigen binding domain that specifically binds CD8, comprises a VH having an amino acid sequence of SEQ ID NO:1017, and a VL having an amino acid sequence of SEQ ID NO:1018. In one embodiment, the first antigen binding domain that specifically binds CD8, comprises a heavy chain having an amino acid sequence of SEQ ID NO:1019. In one embodiment, the first antigen binding domain that specifically binds CD8, comprises a light chain having an amino acid sequence of SEQ ID NO:1020. In one embodiment, the first antigen binding domain that specifically binds CD8, comprises a heavy chain having an amino acid sequence of SEQ ID NO:1019, and a light chain having an amino acid sequence of SEQ ID NO:1020. In one embodiment, the first antigen binding domain that specifically binds CD8, comprises a VH having an amino acid sequence having at least 95% identity to an amino acid sequence of SEQ ID NO:1017. In one embodiment, the first antigen binding domain that specifically binds CD8, comprises a VL having an amino acid sequence having at least 95% identity to an amino acid sequence of SEQ ID NO:1018. In one embodiment, the first antigen binding domain that specifically binds CD8, comprises a VH having an amino acid sequence having at least 95% identity to an amino acid sequence of SEQ ID NO:1017, and a VL having an amino acid sequence having at least 95% identity to an amino acid sequence of SEQ ID NO:1018. In one embodiment, the first antigen binding domain that specifically binds CD8, comprises a heavy chain having an amino acid sequence having at least 95% identity to an amino acid sequence of SEQ ID NO:1019. In one embodiment, the first antigen binding domain that specifically binds CD8, comprises a light chain having an amino acid sequence having at least 95% identity to an amino acid sequence of SEQ ID NO:1020. In one embodiment, the first antigen binding domain that specifically binds CD8, comprises a heavy chain having an amino acid sequence having at least 95% identity to an amino acid sequence of SEQ ID NO:1019, and a light chain having an amino acid sequence having at least 95% identity to an amino acid sequence of SEQ ID NO:1020.

In one embodiment, the first antigen binding domain that specifically binds CD8, comprises: (i) a VH comprising a VH CDR1, a VH CDR2, and a VH CDR3 having an amino acid sequence of SEQ ID NOs:1021, 1022, and 1023, respectively, and (ii) a VL comprising a VL CDR1, VL CDR2, and VL CDR3 having an amino acid sequence of SEQ ID NOs:1024, 1025, and 1026, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1027, 1028, and 1029, respectively, and (ii) a VL comprising a VL CDR1, VL CDR2, and VL CDR3 having an amino acid sequence of SEQ ID NOs:1030, 1031, and 1032, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1033, 1034, and 1035, respectively, and (ii) a VL comprising a VL CDR1, VL CDR2, and VL CDR3 having an amino acid sequence of SEQ ID NOs:1036, 1037, and 1038, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1039, 1040, and 1041, respectively, and (ii) a VL comprising a VL CDR1, VL CDR2, and VL CDR3 having an amino acid sequence of SEQ ID NOs:1042, 1043, and 1044, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1045, 1046, and 1047, respectively, and (ii) a VL comprising a VL CDR1, VL CDR2, and VL CDR3 having an amino acid sequence of SEQ ID NOs:1048, 1049, and 1050, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1051; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1052. In one embodiment, the first antigen binding domain that specifically binds CD8, comprises a VH having an amino acid sequence of SEQ ID NO:1051. In one embodiment, the first antigen binding domain that specifically binds CD8, comprises a VL having an amino acid sequence of SEQ ID NO:1052. In one embodiment, the first antigen binding domain that specifically binds CD8, comprises a VH having an amino acid sequence of SEQ ID NO:1051, and a VL having an amino acid sequence of SEQ ID NO:1052. In one embodiment, the first antigen binding domain that specifically binds CD8, comprises a heavy chain having an amino acid sequence of SEQ ID NO:1053. In one embodiment, the first antigen binding domain that specifically binds CD8, comprises a light chain having an amino acid sequence of SEQ ID NO:1054. In one embodiment, the first antigen binding domain that specifically binds CD8, comprises a heavy chain having an amino acid sequence of SEQ ID NO:1053, and a light chain having an amino acid sequence of SEQ ID NO:1054. In one embodiment, the first antigen binding domain that specifically binds CD8, comprises a VH having an amino acid sequence having at least 95% identity to an amino acid sequence of SEQ ID NO:1051. In one embodiment, the first antigen binding domain that specifically binds CD8, comprises a VL having an amino acid sequence having at least 95% identity to an amino acid sequence of SEQ ID NO:1052. In one embodiment, the first antigen binding domain that specifically binds CD8, comprises a VH having an amino acid sequence having at least 95% identity to an amino acid sequence of SEQ ID NO:1051, and a VL having an amino acid sequence having at least 95% identity to an amino acid sequence of SEQ ID NO:1052. In one embodiment, the first antigen binding domain that specifically binds CD8, comprises a heavy chain having an amino acid sequence having at least 95% identity to an amino acid sequence of SEQ ID NO:1053. In one embodiment, the first antigen binding domain that specifically binds CD8, comprises a light chain having an amino acid sequence having at least 95% identity to an amino acid sequence of SEQ ID NO:1054. In one embodiment, the first antigen binding domain that specifically binds CD8, comprises a heavy chain having an amino acid sequence having at least 95% identity to an amino acid sequence of SEQ ID NO:1053, and a light chain having an amino acid sequence having at least 95% identity to an amino acid sequence of SEQ ID NO:1054.

In one embodiment, the first antigen binding domain that specifically binds CD8, comprises: (i) a VH comprising a VH CDR1, a VH CDR2, and a VH CDR3 having an amino acid sequence of SEQ ID NOs:1055, 1056, and 1057, respectively, and (ii) a VL comprising a VL CDR1, VL CDR2, and VL CDR3 having an amino acid sequence of SEQ ID NOs:1058, 1059, and 1060, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1061, 1062, and 1063, respectively, and (ii) a VL comprising a VL CDR1, VL CDR2, and VL CDR3 having an amino acid sequence of SEQ ID NOs:1064, 1065, and 1066, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1067, 1068, and 1069, respectively, and (ii) a VL comprising a VL CDR1, VL CDR2, and VL CDR3 having an amino acid sequence of SEQ ID NOs:1070, 1071, and 1072, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1073, 1074, and 1075, respectively, and (ii) a VL comprising a VL CDR1, VL CDR2, and VL CDR3 having an amino acid sequence of SEQ ID NOs:1076, 1077, and 1078, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1079, 1080, and 1081, respectively, and (ii) a VL comprising a VL CDR1, VL CDR2, and VL CDR3 having an amino acid sequence of SEQ ID NOs:1082, 1083, and 1084, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1085; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1086. In one embodiment, the first antigen binding domain that specifically binds CD8, comprises a VH having an amino acid sequence of SEQ ID NO:1085. In one embodiment, the first antigen binding domain that specifically binds CD8, comprises a VL having an amino acid sequence of SEQ ID NO:1086. In one embodiment, the first antigen binding domain that specifically binds CD8, comprises a VH having an amino acid sequence of SEQ ID NO:1085, and a VL having an amino acid sequence of SEQ ID NO:1086. In one embodiment, the first antigen binding domain that specifically binds CD8, comprises a heavy chain having an amino acid sequence of SEQ ID NO:1087. In one embodiment, the first antigen binding domain that specifically binds CD8, comprises a light chain having an amino acid sequence of SEQ ID NO:1088. In one embodiment, the first antigen binding domain that specifically binds CD8, comprises a heavy chain having an amino acid sequence of SEQ ID NO:1087, and a light chain having an amino acid sequence of SEQ ID NO:1088. In one embodiment, the first antigen binding domain that specifically binds CD8, comprises a VH having an amino acid sequence having at least 95% identity to an amino acid sequence of SEQ ID NO:1085. In one embodiment, the first antigen binding domain that specifically binds CD8, comprises a VL having an amino acid sequence having at least 95% identity to an amino acid sequence of SEQ ID NO:1086. In one embodiment, the first antigen binding domain that specifically binds CD8, comprises a VH having an amino acid sequence having at least 95% identity to an amino acid sequence of SEQ ID NO:1085, and a VL having an amino acid sequence having at least 95% identity to an amino acid sequence of SEQ ID NO:1086. In one embodiment, the first antigen binding domain that specifically binds CD8, comprises a heavy chain having an amino acid sequence having at least 95% identity to an amino acid sequence of SEQ ID NO:1087. In one embodiment, the first antigen binding domain that specifically binds CD8, comprises a light chain having an amino acid sequence having at least 95% identity to an amino acid sequence of SEQ ID NO:1088. In one embodiment, the first antigen binding domain that specifically binds CD8, comprises a heavy chain having an amino acid sequence having at least 95% identity to an amino acid sequence of SEQ ID NO:1087, and a light chain having an amino acid sequence having at least 95% identity to an amino acid sequence of SEQ ID NO:1088.

In one embodiment, the first antigen binding domain that specifically binds CD8, comprises: (i) a VH comprising a VH CDR1, a VH CDR2, and a VH CDR3 having an amino acid sequence of SEQ ID NOs:1089, 1090, and 1091, respectively, and (ii) a VL comprising a VL CDR1, VL CDR2, and VL CDR3 having an amino acid sequence of SEQ ID NOs:1092, 1093, and 1094, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1095, 1096, and 1097, respectively, and (ii) a VL comprising a VL CDR1, VL CDR2, and VL CDR3 having an amino acid sequence of SEQ ID NOs:1098, 1099, and 1100, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1101, 1102, and 1103, respectively, and (ii) a VL comprising a VL CDR1, VL CDR2, and VL CDR3 having an amino acid sequence of SEQ ID NOs:1104, 1105, and 1106, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1107, 1108, and 1109, respectively, and (ii) a VL comprising a VL CDR1, VL CDR2, and VL CDR3 having an amino acid sequence of SEQ ID NOs:1110, 1111, and 1112, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1113, 1114, and 1115, respectively, and (ii) a VL comprising a VL CDR1, VL CDR2, and VL CDR3 having an amino acid sequence of SEQ ID NOs:1116, 1117, and 1118, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1119; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1120. In one embodiment, the first antigen binding domain that specifically binds CD8, comprises a VH having an amino acid sequence of SEQ ID NO:1119. In one embodiment, the first antigen binding domain that specifically binds CD8, comprises a VL having an amino acid sequence of SEQ ID NO:1120. In one embodiment, the first antigen binding domain that specifically binds CD8, comprises a VH having an amino acid sequence of SEQ ID NO:1119, and a VL having an amino acid sequence of SEQ ID NO:1120. In one embodiment, the first antigen binding domain that specifically binds CD8, comprises a heavy chain having an amino acid sequence of SEQ ID NO:1121. In one embodiment, the first antigen binding domain that specifically binds CD8, comprises a light chain having an amino acid sequence of SEQ ID NO:1122. In one embodiment, the first antigen binding domain that specifically binds CD8, comprises a heavy chain having an amino acid sequence of SEQ ID NO:1121, and a light chain having an amino acid sequence of SEQ ID NO:1122. In one embodiment, the first antigen binding domain that specifically binds CD8, comprises a VH having an amino acid sequence having at least 95% identity to an amino acid sequence of SEQ ID NO:1119. In one embodiment, the first antigen binding domain that specifically binds CD8, comprises a VL having an amino acid sequence having at least 95% identity to an amino acid sequence of SEQ ID NO:1120. In one embodiment, the first antigen binding domain that specifically binds CD8, comprises a VH having an amino acid sequence having at least 95% identity to an amino acid sequence of SEQ ID NO:1119, and a VL having an amino acid sequence having at least 95% identity to an amino acid sequence of SEQ ID NO:1120. In one embodiment, the first antigen binding domain that specifically binds CD8, comprises a heavy chain having an amino acid sequence having at least 95% identity to an amino acid sequence of SEQ ID NO:1121. In one embodiment, the first antigen binding domain that specifically binds CD8, comprises a light chain having an amino acid sequence having at least 95% identity to an amino acid sequence of SEQ ID NO:1122. In one embodiment, the first antigen binding domain that specifically binds CD8, comprises a heavy chain having an amino acid sequence having at least 95% identity to an amino acid sequence of SEQ ID NO:1121, and a light chain having an amino acid sequence having at least 95% identity to an amino acid sequence of SEQ ID NO:1122.

In one embodiment, the first antigen binding domain that specifically binds CD8, comprises: (i) a VH comprising a VH CDR1, a VH CDR2, and a VH CDR3 having an amino acid sequence of SEQ ID NOs:1123, 1124, and 1125, respectively, and (ii) a VL comprising a VL CDR1, VL CDR2, and VL CDR3 having an amino acid sequence of SEQ ID NOs:1126, 1127, and 1128, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1129, 1130, and 1131, respectively, and (ii) a VL comprising a VL CDR1, VL CDR2, and VL CDR3 having an amino acid sequence of SEQ ID NOs:1132, 1133, and 1134, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1135, 1136, and 1137, respectively, and (ii) a VL comprising a VL CDR1, VL CDR2, and VL CDR3 having an amino acid sequence of SEQ ID NOs:1138, 1139, and 1140, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1141, 1142, and 1143, respectively, and (ii) a VL comprising a VL CDR1, VL CDR2, and VL CDR3 having an amino acid sequence of SEQ ID NOs:1144, 1145, and 1146, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1147, 1148, and 1149, respectively, and (ii) a VL comprising a VL CDR1, VL CDR2, and VL CDR3 having an amino acid sequence of SEQ ID NOs:1150, 1151, and 1152, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1153; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1154. In one embodiment, the first antigen binding domain that specifically binds CD8, comprises a VH having an amino acid sequence of SEQ ID NO:1153. In one embodiment, the first antigen binding domain that specifically binds CD8, comprises a VL having an amino acid sequence of SEQ ID NO:1154. In one embodiment, the first antigen binding domain that specifically binds CD8, comprises a VH having an amino acid sequence of SEQ ID NO:1153, and a VL having an amino acid sequence of SEQ ID NO:1154. In one embodiment, the first antigen binding domain that specifically binds CD8, comprises a heavy chain having an amino acid sequence of SEQ ID NO:1155. In one embodiment, the first antigen binding domain that specifically binds CD8, comprises a light chain having an amino acid sequence of SEQ ID NO:1156. In one embodiment, the first antigen binding domain that specifically binds CD8, comprises a heavy chain having an amino acid sequence of SEQ ID NO:1155, and a light chain having an amino acid sequence of SEQ ID NO:1156. In one embodiment, the first antigen binding domain that specifically binds CD8, comprises a VH having an amino acid sequence having at least 95% identity to an amino acid sequence of SEQ ID NO:1153. In one embodiment, the first antigen binding domain that specifically binds CD8, comprises a VL having an amino acid sequence having at least 95% identity to an amino acid sequence of SEQ ID NO:1154. In one embodiment, the first antigen binding domain that specifically binds CD8, comprises a VH having an amino acid sequence having at least 95% identity to an amino acid sequence of SEQ ID NO:1153, and a VL having an amino acid sequence having at least 95% identity to an amino acid sequence of SEQ ID NO:1154. In one embodiment, the first antigen binding domain that specifically binds CD8, comprises a heavy chain having an amino acid sequence having at least 95% identity to an amino acid sequence of SEQ ID NO:1155. In one embodiment, the first antigen binding domain that specifically binds CD8, comprises a light chain having an amino acid sequence having at least 95% identity to an amino acid sequence of SEQ ID NO:1156. In one embodiment, the first antigen binding domain that specifically binds CD8, comprises a heavy chain having an amino acid sequence having at least 95% identity to an amino acid sequence of SEQ ID NO:1155, and a light chain having an amino acid sequence having at least 95% identity to an amino acid sequence of SEQ ID NO:1156.

In one embodiment, the first antigen binding domain that specifically binds CD8, comprises: (i) a VH comprising a VH CDR1, a VH CDR2, and a VH CDR3 having an amino acid sequence of SEQ ID NOs:1157, 1158, and 1159, respectively, and (ii) a VL comprising a VL CDR1, VL CDR2, and VL CDR3 having an amino acid sequence of SEQ ID NOs:1160, 1161, and 1162, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1163, 1164, and 1165, respectively, and (ii) a VL comprising a VL CDR1, VL CDR2, and VL CDR3 having an amino acid sequence of SEQ ID NOs:1166, 1167, and 1168, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1169, 1170, and 1171, respectively, and (ii) a VL comprising a VL CDR1, VL CDR2, and VL CDR3 having an amino acid sequence of SEQ ID NOs:1172, 1173, and 1174, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1175, 1176, and 1177, respectively, and (ii) a VL comprising a VL CDR1, VL CDR2, and VL CDR3 having an amino acid sequence of SEQ ID NOs:1178, 1179, and 1180, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1181, 1182, and 1183, respectively, and (ii) a VL comprising a VL CDR1, VL CDR2, and VL CDR3 having an amino acid sequence of SEQ ID NOs:1184, 1185, and 1186, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1187; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1188. In one embodiment, the first antigen binding domain that specifically binds CD8, comprises a VH having an amino acid sequence of SEQ ID NO:1187. In one embodiment, the first antigen binding domain that specifically binds CD8, comprises a VL having an amino acid sequence of SEQ ID NO:1188. In one embodiment, the first antigen binding domain that specifically binds CD8, comprises a VH having an amino acid sequence of SEQ ID NO:1187, and a VL having an amino acid sequence of SEQ ID NO:1188. In one embodiment, the first antigen binding domain that specifically binds CD8, comprises a heavy chain having an amino acid sequence of SEQ ID NO:1189. In one embodiment, the first antigen binding domain that specifically binds CD8, comprises a light chain having an amino acid sequence of SEQ ID NO:1190. In one embodiment, the first antigen binding domain that specifically binds CD8, comprises a heavy chain having an amino acid sequence of SEQ ID NO:1189, and a light chain having an amino acid sequence of SEQ ID NO:1190. In one embodiment, the first antigen binding domain that specifically binds CD8, comprises a VH having an amino acid sequence having at least 95% identity to an amino acid sequence of SEQ ID NO:1187. In one embodiment, the first antigen binding domain that specifically binds CD8, comprises a VL having an amino acid sequence having at least 95% identity to an amino acid sequence of SEQ ID NO:1188. In one embodiment, the first antigen binding domain that specifically binds CD8, comprises a VH having an amino acid sequence having at least 95% identity to an amino acid sequence of SEQ ID NO:1187, and a VL having an amino acid sequence having at least 95% identity to an amino acid sequence of SEQ ID NO:1188. In one embodiment, the first antigen binding domain that specifically binds CD8, comprises a heavy chain having an amino acid sequence having at least 95% identity to an amino acid sequence of SEQ ID NO:1189. In one embodiment, the first antigen binding domain that specifically binds CD8, comprises a light chain having an amino acid sequence having at least 95% identity to an amino acid sequence of SEQ ID NO:1190. In one embodiment, the first antigen binding domain that specifically binds CD8, comprises a heavy chain having an amino acid sequence having at least 95% identity to an amino acid sequence of SEQ ID NO:1189, and a light chain having an amino acid sequence having at least 95% identity to an amino acid sequence of SEQ ID NO:1190.

In one embodiment, the first antigen binding domain that specifically binds CD8, comprises: (i) a VH comprising a VH CDR1, a VH CDR2, and a VH CDR3 having an amino acid sequence of SEQ ID NOs:1191, 1192, and 1193, respectively, and (ii) a VL comprising a VL CDR1, VL CDR2, and VL CDR3 having an amino acid sequence of SEQ ID NOs:1194, 1195, and 1196, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1197, 1198, and 1199, respectively, and (ii) a VL comprising a VL CDR1, VL CDR2, and VL CDR3 having an amino acid sequence of SEQ ID NOs:1200, 1201, and 1202, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1203, 1204, and 1205, respectively, and (ii) a VL comprising a VL CDR1, VL CDR2, and VL CDR3 having an amino acid sequence of SEQ ID NOs:1206, 1207, and 1208, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1209, 1210, and 1211, respectively, and (ii) a VL comprising a VL CDR1, VL CDR2, and VL CDR3 having an amino acid sequence of SEQ ID NOs:1212, 1213, and 1214, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1215, 1216, and 1217, respectively, and (ii) a VL comprising a VL CDR1, VL CDR2, and VL CDR3 having an amino acid sequence of SEQ ID NOs:1218, 1219, and 1220, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1221; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1222. In one embodiment, the first antigen binding domain that specifically binds CD8, comprises a VH having an amino acid sequence of SEQ ID NO:1221. In one embodiment, the first antigen binding domain that specifically binds CD8, comprises a VL having an amino acid sequence of SEQ ID NO:1222. In one embodiment, the first antigen binding domain that specifically binds CD8, comprises a VH having an amino acid sequence of SEQ ID NO:1221, and a VL having an amino acid sequence of SEQ ID NO:1222. In one embodiment, the first antigen binding domain that specifically binds CD8, comprises a heavy chain having an amino acid sequence of SEQ ID NO:1223. In one embodiment, the first antigen binding domain that specifically binds CD8, comprises a light chain having an amino acid sequence of SEQ ID NO:1224. In one embodiment, the first antigen binding domain that specifically binds CD8, comprises a heavy chain having an amino acid sequence of SEQ ID NO:1223, and a light chain having an amino acid sequence of SEQ ID NO:1224. In one embodiment, the first antigen binding domain that specifically binds CD8, comprises a VH having an amino acid sequence having at least 95% identity to an amino acid sequence of SEQ ID NO:1221. In one embodiment, the first antigen binding domain that specifically binds CD8, comprises a VL having an amino acid sequence having at least 95% identity to an amino acid sequence of SEQ ID NO:1222. In one embodiment, the first antigen binding domain that specifically binds CD8, comprises a VH having an amino acid sequence having at least 95% identity to an amino acid sequence of SEQ ID NO:1221, and a VL having an amino acid sequence having at least 95% identity to an amino acid sequence of SEQ ID NO:1222. In one embodiment, the first antigen binding domain that specifically binds CD8, comprises a heavy chain having an amino acid sequence having at least 95% identity to an amino acid sequence of SEQ ID NO:1223. In one embodiment, the first antigen binding domain that specifically binds CD8, comprises a light chain having an amino acid sequence having at least 95% identity to an amino acid sequence of SEQ ID NO:1224. In one embodiment, the first antigen binding domain that specifically binds CD8, comprises a heavy chain having an amino acid sequence having at least 95% identity to an amino acid sequence of SEQ ID NO:1223, and a light chain having an amino acid sequence having at least 95% identity to an amino acid sequence of SEQ ID NO:1224.

In one embodiment, the first antigen binding domain that specifically binds CD8, comprises: (i) a VH comprising a VH CDR1, a VH CDR2, and a VH CDR3 having an amino acid sequence of SEQ ID NOs:1225, 1226, and 1227, respectively, and (ii) a VL comprising a VL CDR1, VL CDR2, and VL CDR3 having an amino acid sequence of SEQ ID NOs:1228, 1229, and 1230, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1231, 1232, and 1233, respectively, and (ii) a VL comprising a VL CDR1, VL CDR2, and VL CDR3 having an amino acid sequence of SEQ ID NOs:1234, 1235, and 1236, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1237, 1238, and 1239, respectively, and (ii) a VL comprising a VL CDR1, VL CDR2, and VL CDR3 having an amino acid sequence of SEQ ID NOs:1240, 1241, and 1242, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1243, 1244, and 1245, respectively, and (ii) a VL comprising a VL CDR1, VL CDR2, and VL CDR3 having an amino acid sequence of SEQ ID NOs:1246, 1247, and 1248, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1249, 1250, and 1251, respectively, and (ii) a VL comprising a VL CDR1, VL CDR2, and VL CDR3 having an amino acid sequence of SEQ ID NOs:1252, 1253, and 1254, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1255; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1256. In one embodiment, the first antigen binding domain that specifically binds CD8, comprises a VH having an amino acid sequence of SEQ ID NO:1255. In one embodiment, the first antigen binding domain that specifically binds CD8, comprises a VL having an amino acid sequence of SEQ ID NO:1256. In one embodiment, the first antigen binding domain that specifically binds CD8, comprises a VH having an amino acid sequence of SEQ ID NO:1255, and a VL having an amino acid sequence of SEQ ID NO:1256. In one embodiment, the first antigen binding domain that specifically binds CD8, comprises a heavy chain having an amino acid sequence of SEQ ID NO:1257. In one embodiment, the first antigen binding domain that specifically binds CD8, comprises a light chain having an amino acid sequence of SEQ ID NO:1258. In one embodiment, the first antigen binding domain that specifically binds CD8, comprises a heavy chain having an amino acid sequence of SEQ ID NO:1257, and a light chain having an amino acid sequence of SEQ ID NO:1258. In one embodiment, the first antigen binding domain that specifically binds CD8, comprises a VH having an amino acid sequence having at least 95% identity to an amino acid sequence of SEQ ID NO:1255. In one embodiment, the first antigen binding domain that specifically binds CD8, comprises a VL having an amino acid sequence having at least 95% identity to an amino acid sequence of SEQ ID NO:1256. In one embodiment, the first antigen binding domain that specifically binds CD8, comprises a VH having an amino acid sequence having at least 95% identity to an amino acid sequence of SEQ ID NO:1255, and a VL having an amino acid sequence having at least 95% identity to an amino acid sequence of SEQ ID NO:1256. In one embodiment, the first antigen binding domain that specifically binds CD8, comprises a heavy chain having an amino acid sequence having at least 95% identity to an amino acid sequence of SEQ ID NO:1257. In one embodiment, the first antigen binding domain that specifically binds CD8, comprises a light chain having an amino acid sequence having at least 95% identity to an amino acid sequence of SEQ ID NO:1258. In one embodiment, the first antigen binding domain that specifically binds CD8, comprises a heavy chain having an amino acid sequence having at least 95% identity to an amino acid sequence of SEQ ID NO:1257, and a light chain having an amino acid sequence having at least 95% identity to an amino acid sequence of SEQ ID NO:1258.

In one embodiment, the first antigen binding domain that specifically binds CD8, comprises: (i) a VH comprising a VH CDR1, a VH CDR2, and a VH CDR3 having an amino acid sequence of SEQ ID NOs:1259, 1260, and 1261, respectively, and (ii) a VL comprising a VL CDR1, VL CDR2, and VL CDR3 having an amino acid sequence of SEQ ID NOs:1262, 1263, and 1264, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1265, 1266, and 1267, respectively, and (ii) a VL comprising a VL CDR1, VL CDR2, and VL CDR3 having an amino acid sequence of SEQ ID NOs:1268, 1269, and 1270, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1271, 1272, and 1273, respectively, and (ii) a VL comprising a VL CDR1, VL CDR2, and VL CDR3 having an amino acid sequence of SEQ ID NOs:1274, 1275, and 1276, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1277, 1278, and 1279, respectively, and (ii) a VL comprising a VL CDR1, VL CDR2, and VL CDR3 having an amino acid sequence of SEQ ID NOs:1280, 1281, and 1282, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1283, 1284, and 1285, respectively, and (ii) a VL comprising a VL CDR1, VL CDR2, and VL CDR3 having an amino acid sequence of SEQ ID NOs:1286, 1287, and 1288, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1289; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1290. In one embodiment, the first antigen binding domain that specifically binds CD8, comprises a VH having an amino acid sequence of SEQ ID NO:1289. In one embodiment, the first antigen binding domain that specifically binds CD8, comprises a VL having an amino acid sequence of SEQ ID NO:1290. In one embodiment, the first antigen binding domain that specifically binds CD8, comprises a VH having an amino acid sequence of SEQ ID NO:1289, and a VL having an amino acid sequence of SEQ ID NO:1290. In one embodiment, the first antigen binding domain that specifically binds CD8, comprises a heavy chain having an amino acid sequence of SEQ ID NO:1291. In one embodiment, the first antigen binding domain that specifically binds CD8, comprises a light chain having an amino acid sequence of SEQ ID NO:1292. In one embodiment, the first antigen binding domain that specifically binds CD8, comprises a heavy chain having an amino acid sequence of SEQ ID NO:1291, and a light chain having an amino acid sequence of SEQ ID NO:1292. In one embodiment, the first antigen binding domain that specifically binds CD8, comprises a VH having an amino acid sequence having at least 95% identity to an amino acid sequence of SEQ ID NO:1289. In one embodiment, the first antigen binding domain that specifically binds CD8, comprises a VL having an amino acid sequence having at least 95% identity to an amino acid sequence of SEQ ID NO:1290. In one embodiment, the first antigen binding domain that specifically binds CD8, comprises a VH having an amino acid sequence having at least 95% identity to an amino acid sequence of SEQ ID NO:1289, and a VL having an amino acid sequence having at least 95% identity to an amino acid sequence of SEQ ID NO:1290. In one embodiment, the first antigen binding domain that specifically binds CD8, comprises a heavy chain having an amino acid sequence having at least 95% identity to an amino acid sequence of SEQ ID NO:1291. In one embodiment, the first antigen binding domain that specifically binds CD8, comprises a light chain having an amino acid sequence having at least 95% identity to an amino acid sequence of SEQ ID NO:1292. In one embodiment, the first antigen binding domain that specifically binds CD8, comprises a heavy chain having an amino acid sequence having at least 95% identity to an amino acid sequence of SEQ ID NO:1291, and a light chain having an amino acid sequence having at least 95% identity to an amino acid sequence of SEQ ID NO:1292.

In one embodiment, the first antigen binding domain that specifically binds CD8, comprises: (i) a VH comprising a VH CDR1, a VH CDR2, and a VH CDR3 having an amino acid sequence of SEQ ID NOs:1293, 1294, and 1295, respectively, and (ii) a VL comprising a VL CDR1, VL CDR2, and VL CDR3 having an amino acid sequence of SEQ ID NOs:1296, 1297, and 1298, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1299, 1300, and 1301, respectively, and (ii) a VL comprising a VL CDR1, VL CDR2, and VL CDR3 having an amino acid sequence of SEQ ID NOs:1302, 1303, and 1304, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1305, 1306, and 1307, respectively, and (ii) a VL comprising a VL CDR1, VL CDR2, and VL CDR3 having an amino acid sequence of SEQ ID NOs:1308, 1309, and 1310, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1311, 1312, and 1313, respectively, and (ii) a VL comprising a VL CDR1, VL CDR2, and VL CDR3 having an amino acid sequence of SEQ ID NOs:1314, 1315, and 1316, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1317, 1318, and 1319, respectively, and (ii) a VL comprising a VL CDR1, VL CDR2, and VL CDR3 having an amino acid sequence of SEQ ID NOs:1320, 1321, and 1322, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1323; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1324. In one embodiment, the first antigen binding domain that specifically binds CD8, comprises a VH having an amino acid sequence of SEQ ID NO:1323. In one embodiment, the first antigen binding domain that specifically binds CD8, comprises a VL having an amino acid sequence of SEQ ID NO:1324. In one embodiment, the first antigen binding domain that specifically binds CD8, comprises a VH having an amino acid sequence of SEQ ID NO:1323, and a VL having an amino acid sequence of SEQ ID NO:1324. In one embodiment, the first antigen binding domain that specifically binds CD8, comprises a heavy chain having an amino acid sequence of SEQ ID NO:1325. In one embodiment, the first antigen binding domain that specifically binds CD8, comprises a light chain having an amino acid sequence of SEQ ID NO:1326. In one embodiment, the first antigen binding domain that specifically binds CD8, comprises a heavy chain having an amino acid sequence of SEQ ID NO:1325, and a light chain having an amino acid sequence of SEQ ID NO:1326. In one embodiment, the first antigen binding domain that specifically binds CD8, comprises a VH having an amino acid sequence having at least 95% identity to an amino acid sequence of SEQ ID NO:1323. In one embodiment, the first antigen binding domain that specifically binds CD8, comprises a VL having an amino acid sequence having at least 95% identity to an amino acid sequence of SEQ ID NO:1324. In one embodiment, the first antigen binding domain that specifically binds CD8, comprises a VH having an amino acid sequence having at least 95% identity to an amino acid sequence of SEQ ID NO:1323, and a VL having an amino acid sequence having at least 95% identity to an amino acid sequence of SEQ ID NO:1324. In one embodiment, the first antigen binding domain that specifically binds CD8, comprises a heavy chain having an amino acid sequence having at least 95% identity to an amino acid sequence of SEQ ID NO:1325. In one embodiment, the first antigen binding domain that specifically binds CD8, comprises a light chain having an amino acid sequence having at least 95% identity to an amino acid sequence of SEQ ID NO:1326. In one embodiment, the first antigen binding domain that specifically binds CD8, comprises a heavy chain having an amino acid sequence having at least 95% identity to an amino acid sequence of SEQ ID NO:1325, and a light chain having an amino acid sequence having at least 95% identity to an amino acid sequence of SEQ ID NO:1326.

In one embodiment, the first antigen binding domain that specifically binds CD8, comprises: (i) a VH comprising a VH CDR1, a VH CDR2, and a VH CDR3 having an amino acid sequence of SEQ ID NOs:1327, 1328, and 1329, respectively, and (ii) a VL comprising a VL CDR1, VL CDR2, and VL CDR3 having an amino acid sequence of SEQ ID NOs:1330, 1331, and 1332, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1333, 1334, and 1335, respectively, and (ii) a VL comprising a VL CDR1, VL CDR2, and VL CDR3 having an amino acid sequence of SEQ ID NOs:1336, 1337, and 1338, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1339, 1340, and 1341, respectively, and (ii) a VL comprising a VL CDR1, VL CDR2, and VL CDR3 having an amino acid sequence of SEQ ID NOs:1342, 1343, and 1344, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1345, 1346, and 1347, respectively, and (ii) a VL comprising a VL CDR1, VL CDR2, and VL CDR3 having an amino acid sequence of SEQ ID NOs:1348, 1349, and 1350, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1351, 1352, and 1353, respectively, and (ii) a VL comprising a VL CDR1, VL CDR2, and VL CDR3 having an amino acid sequence of SEQ ID NOs:1354, 1355, and 1356, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1357; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1358. In one embodiment, the first antigen binding domain that specifically binds CD8, comprises a VH having an amino acid sequence of SEQ ID NO:1357. In one embodiment, the first antigen binding domain that specifically binds CD8, comprises a VL having an amino acid sequence of SEQ ID NO:1358. In one embodiment, the first antigen binding domain that specifically binds CD8, comprises a VH having an amino acid sequence of SEQ ID NO:1357, and a VL having an amino acid sequence of SEQ ID NO:1358. In one embodiment, the first antigen binding domain that specifically binds CD8, comprises a heavy chain having an amino acid sequence of SEQ ID NO:1359. In one embodiment, the first antigen binding domain that specifically binds CD8, comprises a light chain having an amino acid sequence of SEQ ID NO:1360. In one embodiment, the first antigen binding domain that specifically binds CD8, comprises a heavy chain having an amino acid sequence of SEQ ID NO:1359, and a light chain having an amino acid sequence of SEQ ID NO:1360. In one embodiment, the first antigen binding domain that specifically binds CD8, comprises a VH having an amino acid sequence having at least 95% identity to an amino acid sequence of SEQ ID NO:1357. In one embodiment, the first antigen binding domain that specifically binds CD8, comprises a VL having an amino acid sequence having at least 95% identity to an amino acid sequence of SEQ ID NO:1358. In one embodiment, the first antigen binding domain that specifically binds CD8, comprises a VH having an amino acid sequence having at least 95% identity to an amino acid sequence of SEQ ID NO:1357, and a VL having an amino acid sequence having at least 95% identity to an amino acid sequence of SEQ ID NO:1358. In one embodiment, the first antigen binding domain that specifically binds CD8, comprises a heavy chain having an amino acid sequence having at least 95% identity to an amino acid sequence of SEQ ID NO:1359. In one embodiment, the first antigen binding domain that specifically binds CD8, comprises a light chain having an amino acid sequence having at least 95% identity to an amino acid sequence of SEQ ID NO:1360. In one embodiment, the first antigen binding domain that specifically binds CD8, comprises a heavy chain having an amino acid sequence having at least 95% identity to an amino acid sequence of SEQ ID NO:1359, and a light chain having an amino acid sequence having at least 95% identity to an amino acid sequence of SEQ ID NO:1360.

In one embodiment, the first antigen binding domain that specifically binds CD8, comprises: (i) a VH comprising a VH CDR1, a VH CDR2, and a VH CDR3 having an amino acid sequence of SEQ ID NOs:1361, 1362, and 1363, respectively, and (ii) a VL comprising a VL CDR1, VL CDR2, and VL CDR3 having an amino acid sequence of SEQ ID NOs:1364, 1365, and 1366, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1367, 1368, and 1369, respectively, and (ii) a VL comprising a VL CDR1, VL CDR2, and VL CDR3 having an amino acid sequence of SEQ ID NOs:1370, 1371, and 1372, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1373, 1374, and 1375, respectively, and (ii) a VL comprising a VL CDR1, VL CDR2, and VL CDR3 having an amino acid sequence of SEQ ID NOs:1376, 1377, and 1378, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1379, 1380, and 1381, respectively, and (ii) a VL comprising a VL CDR1, VL CDR2, and VL CDR3 having an amino acid sequence of SEQ ID NOs:1382, 1383, and 1384, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1385, 1386, and 1387, respectively, and (ii) a VL comprising a VL CDR1, VL CDR2, and VL CDR3 having an amino acid sequence of SEQ ID NOs:1388, 1389, and 1390, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1391; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1392. In one embodiment, the first antigen binding domain that specifically binds CD8, comprises a VH having an amino acid sequence of SEQ ID NO:1391. In one embodiment, the first antigen binding domain that specifically binds CD8, comprises a VL having an amino acid sequence of SEQ ID NO:1392. In one embodiment, the first antigen binding domain that specifically binds CD8, comprises a VH having an amino acid sequence of SEQ ID NO:1391, and a VL having an amino acid sequence of SEQ ID NO:1392. In one embodiment, the first antigen binding domain that specifically binds CD8, comprises a heavy chain having an amino acid sequence of SEQ ID NO:1393. In one embodiment, the first antigen binding domain that specifically binds CD8, comprises a light chain having an amino acid sequence of SEQ ID NO:1394. In one embodiment, the first antigen binding domain that specifically binds CD8, comprises a heavy chain having an amino acid sequence of SEQ ID NO:1393, and a light chain having an amino acid sequence of SEQ ID NO:1394. In one embodiment, the first antigen binding domain that specifically binds CD8, comprises a VH having an amino acid sequence having at least 95% identity to an amino acid sequence of SEQ ID NO:1391. In one embodiment, the first antigen binding domain that specifically binds CD8, comprises a VL having an amino acid sequence having at least 95% identity to an amino acid sequence of SEQ ID NO:1392. In one embodiment, the first antigen binding domain that specifically binds CD8, comprises a VH having an amino acid sequence having at least 95% identity to an amino acid sequence of SEQ ID NO:1391, and a VL having an amino acid sequence having at least 95% identity to an amino acid sequence of SEQ ID NO:1392. In one embodiment, the first antigen binding domain that specifically binds CD8, comprises a heavy chain having an amino acid sequence having at least 95% identity to an amino acid sequence of SEQ ID NO:1393. In one embodiment, the first antigen binding domain that specifically binds CD8, comprises a light chain having an amino acid sequence having at least 95% identity to an amino acid sequence of SEQ ID NO:1394. In one embodiment, the first antigen binding domain that specifically binds CD8, comprises a heavy chain having an amino acid sequence having at least 95% identity to an amino acid sequence of SEQ ID NO:1393, and a light chain having an amino acid sequence having at least 95% identity to an amino acid sequence of SEQ ID NO:1394.

In one embodiment, the first antigen binding domain that specifically binds CD8, comprises: (i) a VH comprising a VH CDR1, a VH CDR2, and a VH CDR3 having an amino acid sequence of SEQ ID NOs:1395, 1396, and 1397, respectively, and (ii) a VL comprising a VL CDR1, VL CDR2, and VL CDR3 having an amino acid sequence of SEQ ID NOs:1398, 1399, and 1400, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1401, 1402, and 1403, respectively, and (ii) a VL comprising a VL CDR1, VL CDR2, and VL CDR3 having an amino acid sequence of SEQ ID NOs:1404, 1405, and 1406, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1407, 1408, and 1409, respectively, and (ii) a VL comprising a VL CDR1, VL CDR2, and VL CDR3 having an amino acid sequence of SEQ ID NOs:1410, 1411, and 1412, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1413, 1414, and 1415, respectively, and (ii) a VL comprising a VL CDR1, VL CDR2, and VL CDR3 having an amino acid sequence of SEQ ID NOs:1416, 1417, and 1418, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1419, 1420, and 1421, respectively, and (ii) a VL comprising a VL CDR1, VL CDR2, and VL CDR3 having an amino acid sequence of SEQ ID NOs:1422, 1423, and 1424, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1425; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1426. In one embodiment, the first antigen binding domain that specifically binds CD8, comprises a VH having an amino acid sequence of SEQ ID NO:1425. In one embodiment, the first antigen binding domain that specifically binds CD8, comprises a VL having an amino acid sequence of SEQ ID NO:1426. In one embodiment, the first antigen binding domain that specifically binds CD8, comprises a VH having an amino acid sequence of SEQ ID NO:1425, and a VL having an amino acid sequence of SEQ ID NO:1426. In one embodiment, the first antigen binding domain that specifically binds CD8, comprises a heavy chain having an amino acid sequence of SEQ ID NO:1427. In one embodiment, the first antigen binding domain that specifically binds CD8, comprises a light chain having an amino acid sequence of SEQ ID NO:1428. In one embodiment, the first antigen binding domain that specifically binds CD8, comprises a heavy chain having an amino acid sequence of SEQ ID NO:1427, and a light chain having an amino acid sequence of SEQ ID NO:1428. In one embodiment, the first antigen binding domain that specifically binds CD8, comprises a VH having an amino acid sequence having at least 95% identity to an amino acid sequence of SEQ ID NO:1425. In one embodiment, the first antigen binding domain that specifically binds CD8, comprises a VL having an amino acid sequence having at least 95% identity to an amino acid sequence of SEQ ID NO:1426. In one embodiment, the first antigen binding domain that specifically binds CD8, comprises a VH having an amino acid sequence having at least 95% identity to an amino acid sequence of SEQ ID NO:1425, and a VL having an amino acid sequence having at least 95% identity to an amino acid sequence of SEQ ID NO:1426. In one embodiment, the first antigen binding domain that specifically binds CD8, comprises a heavy chain having an amino acid sequence having at least 95% identity to an amino acid sequence of SEQ ID NO:1427. In one embodiment, the first antigen binding domain that specifically binds CD8, comprises a light chain having an amino acid sequence having at least 95% identity to an amino acid sequence of SEQ ID NO:1428. In one embodiment, the first antigen binding domain that specifically binds CD8, comprises a heavy chain having an amino acid sequence having at least 95% identity to an amino acid sequence of SEQ ID NO:1427, and a light chain having an amino acid sequence having at least 95% identity to an amino acid sequence of SEQ ID NO:1428.

In one embodiment, the first antigen binding domain that specifically binds CD8, comprises: (i) a VH comprising a VH CDR1, a VH CDR2, and a VH CDR3 having an amino acid sequence of SEQ ID NOs:1429, 1430, and 1431, respectively, and (ii) a VL comprising a VL CDR1, VL CDR2, and VL CDR3 having an amino acid sequence of SEQ ID NOs:1432, 1433, and 1434, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1435, 1436, and 1437, respectively, and (ii) a VL comprising a VL CDR1, VL CDR2, and VL CDR3 having an amino acid sequence of SEQ ID NOs:1438, 1439, and 1440, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1441, 1442, and 1443, respectively, and (ii) a VL comprising a VL CDR1, VL CDR2, and VL CDR3 having an amino acid sequence of SEQ ID NOs:1444, 1445, and 1446, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1447, 1448, and 1449, respectively, and (ii) a VL comprising a VL CDR1, VL CDR2, and VL CDR3 having an amino acid sequence of SEQ ID NOs:1450, 1451, and 1452, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1453, 1454, and 1455, respectively, and (ii) a VL comprising a VL CDR1, VL CDR2, and VL CDR3 having an amino acid sequence of SEQ ID NOs:1456, 1457, and 1458, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1459; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1460. In one embodiment, the first antigen binding domain that specifically binds CD8, comprises a VH having an amino acid sequence of SEQ ID NO:1459. In one embodiment, the first antigen binding domain that specifically binds CD8, comprises a VL having an amino acid sequence of SEQ ID NO:1460. In one embodiment, the first antigen binding domain that specifically binds CD8, comprises a VH having an amino acid sequence of SEQ ID NO:1459, and a VL having an amino acid sequence of SEQ ID NO:1460. In one embodiment, the first antigen binding domain that specifically binds CD8, comprises a heavy chain having an amino acid sequence of SEQ ID NO:1461. In one embodiment, the first antigen binding domain that specifically binds CD8, comprises a light chain having an amino acid sequence of SEQ ID NO:1462. In one embodiment, the first antigen binding domain that specifically binds CD8, comprises a heavy chain having an amino acid sequence of SEQ ID NO:1461, and a light chain having an amino acid sequence of SEQ ID NO:1462. In one embodiment, the first antigen binding domain that specifically binds CD8, comprises a VH having an amino acid sequence having at least 95% identity to an amino acid sequence of SEQ ID NO:1459. In one embodiment, the first antigen binding domain that specifically binds CD8, comprises a VL having an amino acid sequence having at least 95% identity to an amino acid sequence of SEQ ID NO:1460. In one embodiment, the first antigen binding domain that specifically binds CD8, comprises a VH having an amino acid sequence having at least 95% identity to an amino acid sequence of SEQ ID NO:1459, and a VL having an amino acid sequence having at least 95% identity to an amino acid sequence of SEQ ID NO:1460. In one embodiment, the first antigen binding domain that specifically binds CD8, comprises a heavy chain having an amino acid sequence having at least 95% identity to an amino acid sequence of SEQ ID NO:1461. In one embodiment, the first antigen binding domain that specifically binds CD8, comprises a light chain having an amino acid sequence having at least 95% identity to an amino acid sequence of SEQ ID NO:1462. In one embodiment, the first antigen binding domain that specifically binds CD8, comprises a heavy chain having an amino acid sequence having at least 95% identity to an amino acid sequence of SEQ ID NO:1461, and a light chain having an amino acid sequence having at least 95% identity to an amino acid sequence of SEQ ID NO:1462.

In one embodiment, the first antigen binding domain that specifically binds CD8, comprises: (i) a VH comprising a VH CDR1, a VH CDR2, and a VH CDR3 having an amino acid sequence of SEQ ID NOs:1463, 1464, and 1465, respectively, and (ii) a VL comprising a VL CDR1, VL CDR2, and VL CDR3 having an amino acid sequence of SEQ ID NOs:1466, 1467, and 1468, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1469, 1470, and 1471, respectively, and (ii) a VL comprising a VL CDR1, VL CDR2, and VL CDR3 having an amino acid sequence of SEQ ID NOs:1472, 1473, and 1474, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1475, 1476, and 1477, respectively, and (ii) a VL comprising a VL CDR1, VL CDR2, and VL CDR3 having an amino acid sequence of SEQ ID NOs:1478, 1479, and 1480, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1481, 1482, and 1483, respectively, and (ii) a VL comprising a VL CDR1, VL CDR2, and VL CDR3 having an amino acid sequence of SEQ ID NOs:1484, 1485, and 1486, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1487, 1488, and 1489, respectively, and (ii) a VL comprising a VL CDR1, VL CDR2, and VL CDR3 having an amino acid sequence of SEQ ID NOs:1490, 1491, and 1492, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1493; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1494. In one embodiment, the first antigen binding domain that specifically binds CD8, comprises a VH having an amino acid sequence of SEQ ID NO:1493. In one embodiment, the first antigen binding domain that specifically binds CD8, comprises a VL having an amino acid sequence of SEQ ID NO:1494. In one embodiment, the first antigen binding domain that specifically binds CD8, comprises a VH having an amino acid sequence of SEQ ID NO:1493, and a VL having an amino acid sequence of SEQ ID NO:1494. In one embodiment, the first antigen binding domain that specifically binds CD8, comprises a heavy chain having an amino acid sequence of SEQ ID NO:1495. In one embodiment, the first antigen binding domain that specifically binds CD8, comprises a light chain having an amino acid sequence of SEQ ID NO:1496. In one embodiment, the first antigen binding domain that specifically binds CD8, comprises a heavy chain having an amino acid sequence of SEQ ID NO:1495, and a light chain having an amino acid sequence of SEQ ID NO:1496. In one embodiment, the first antigen binding domain that specifically binds CD8, comprises a VH having an amino acid sequence having at least 95% identity to an amino acid sequence of SEQ ID NO:1493. In one embodiment, the first antigen binding domain that specifically binds CD8, comprises a VL having an amino acid sequence having at least 95% identity to an amino acid sequence of SEQ ID NO:1494. In one embodiment, the first antigen binding domain that specifically binds CD8, comprises a VH having an amino acid sequence having at least 95% identity to an amino acid sequence of SEQ ID NO:1493, and a VL having an amino acid sequence having at least 95% identity to an amino acid sequence of SEQ ID NO:1494. In one embodiment, the first antigen binding domain that specifically binds CD8, comprises a heavy chain having an amino acid sequence having at least 95% identity to an amino acid sequence of SEQ ID NO:1495. In one embodiment, the first antigen binding domain that specifically binds CD8, comprises a light chain having an amino acid sequence having at least 95% identity to an amino acid sequence of SEQ ID NO:1496. In one embodiment, the first antigen binding domain that specifically binds CD8, comprises a heavy chain having an amino acid sequence having at least 95% identity to an amino acid sequence of SEQ ID NO:1495, and a light chain having an amino acid sequence having at least 95% identity to an amino acid sequence of SEQ ID NO:1496.

In one embodiment, the first antigen binding domain that specifically binds CD8, comprises: (i) a VH comprising a VH CDR1, a VH CDR2, and a VH CDR3 having an amino acid sequence of SEQ ID NOs:1497, 1498, and 1499, respectively, and (ii) a VL comprising a VL CDR1, VL CDR2, and VL CDR3 having an amino acid sequence of SEQ ID NOs:1500, 1501, and 1502, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1503, 1504, and 1505, respectively, and (ii) a VL comprising a VL CDR1, VL CDR2, and VL CDR3 having an amino acid sequence of SEQ ID NOs:1506, 1507, and 1508, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1509, 1510, and 1511, respectively, and (ii) a VL comprising a VL CDR1, VL CDR2, and VL CDR3 having an amino acid sequence of SEQ ID NOs:1512, 1513, and 1514, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1515, 1516, and 1517, respectively, and (ii) a VL comprising a VL CDR1, VL CDR2, and VL CDR3 having an amino acid sequence of SEQ ID NOs:1518, 1519, and 1520, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1521, 1522, and 1523, respectively, and (ii) a VL comprising a VL CDR1, VL CDR2, and VL CDR3 having an amino acid sequence of SEQ ID NOs:1524, 1525, and 1526, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1527; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1528. In one embodiment, the first antigen binding domain that specifically binds CD8, comprises a VH having an amino acid sequence of SEQ ID NO:1527. In one embodiment, the first antigen binding domain that specifically binds CD8, comprises a VL having an amino acid sequence of SEQ ID NO:1528. In one embodiment, the first antigen binding domain that specifically binds CD8, comprises a VH having an amino acid sequence of SEQ ID NO:1527, and a VL having an amino acid sequence of SEQ ID NO:1528. In one embodiment, the first antigen binding domain that specifically binds CD8, comprises a heavy chain having an amino acid sequence of SEQ ID NO:1529. In one embodiment, the first antigen binding domain that specifically binds CD8, comprises a light chain having an amino acid sequence of SEQ ID NO:1530. In one embodiment, the first antigen binding domain that specifically binds CD8, comprises a heavy chain having an amino acid sequence of SEQ ID NO:1529, and a light chain having an amino acid sequence of SEQ ID NO:1530. In one embodiment, the first antigen binding domain that specifically binds CD8, comprises a VH having an amino acid sequence having at least 95% identity to an amino acid sequence of SEQ ID NO:1527. In one embodiment, the first antigen binding domain that specifically binds CD8, comprises a VL having an amino acid sequence having at least 95% identity to an amino acid sequence of SEQ ID NO:1528. In one embodiment, the first antigen binding domain that specifically binds CD8, comprises a VH having an amino acid sequence having at least 95% identity to an amino acid sequence of SEQ ID NO:1527, and a VL having an amino acid sequence having at least 95% identity to an amino acid sequence of SEQ ID NO:1528. In one embodiment, the first antigen binding domain that specifically binds CD8, comprises a heavy chain having an amino acid sequence having at least 95% identity to an amino acid sequence of SEQ ID NO:1529. In one embodiment, the first antigen binding domain that specifically binds CD8, comprises a light chain having an amino acid sequence having at least 95% identity to an amino acid sequence of SEQ ID NO:1530. In one embodiment, the first antigen binding domain that specifically binds CD8, comprises a heavy chain having an amino acid sequence having at least 95% identity to an amino acid sequence of SEQ ID NO:1529, and a light chain having an amino acid sequence having at least 95% identity to an amino acid sequence of SEQ ID NO:1530.

In one embodiment, the first antigen binding domain that specifically binds CD8, comprises: (i) a VH comprising a VH CDR1, a VH CDR2, and a VH CDR3 having an amino acid sequence of SEQ ID NOs:1531, 1532, and 1533, respectively, and (ii) a VL comprising a VL CDR1, VL CDR2, and VL CDR3 having an amino acid sequence of SEQ ID NOs:1534, 1535, and 1536, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1537, 1538, and 1539, respectively, and (ii) a VL comprising a VL CDR1, VL CDR2, and VL CDR3 having an amino acid sequence of SEQ ID NOs:1540, 1541, and 1542, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1543, 1544, and 1545, respectively, and (ii) a VL comprising a VL CDR1, VL CDR2, and VL CDR3 having an amino acid sequence of SEQ ID NOs:1546, 1547, and 1548, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1549, 1550, and 1551, respectively, and (ii) a VL comprising a VL CDR1, VL CDR2, and VL CDR3 having an amino acid sequence of SEQ ID NOs:1552, 1553, and 1554, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1555, 1556, and 1557, respectively, and (ii) a VL comprising a VL CDR1, VL CDR2, and VL CDR3 having an amino acid sequence of SEQ ID NOs:1558, 1559, and 1560, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1561; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1562. In one embodiment, the first antigen binding domain that specifically binds CD8, comprises a VH having an amino acid sequence of SEQ ID NO:1561. In one embodiment, the first antigen binding domain that specifically binds CD8, comprises a VL having an amino acid sequence of SEQ ID NO:1562. In one embodiment, the first antigen binding domain that specifically binds CD8, comprises a VH having an amino acid sequence of SEQ ID NO:1561, and a VL having an amino acid sequence of SEQ ID NO:1562. In one embodiment, the first antigen binding domain that specifically binds CD8, comprises a heavy chain having an amino acid sequence of SEQ ID NO:1563. In one embodiment, the first antigen binding domain that specifically binds CD8, comprises a light chain having an amino acid sequence of SEQ ID NO:1564. In one embodiment, the first antigen binding domain that specifically binds CD8, comprises a heavy chain having an amino acid sequence of SEQ ID NO:1563, and a light chain having an amino acid sequence of SEQ ID NO:1564. In one embodiment, the first antigen binding domain that specifically binds CD8, comprises a VH having an amino acid sequence having at least 95% identity to an amino acid sequence of SEQ ID NO:1561. In one embodiment, the first antigen binding domain that specifically binds CD8, comprises a VL having an amino acid sequence having at least 95% identity to an amino acid sequence of SEQ ID NO:1562. In one embodiment, the first antigen binding domain that specifically binds CD8, comprises a VH having an amino acid sequence having at least 95% identity to an amino acid sequence of SEQ ID NO:1561, and a VL having an amino acid sequence having at least 95% identity to an amino acid sequence of SEQ ID NO:1562. In one embodiment, the first antigen binding domain that specifically binds CD8, comprises a heavy chain having an amino acid sequence having at least 95% identity to an amino acid sequence of SEQ ID NO:1563. In one embodiment, the first antigen binding domain that specifically binds CD8, comprises a light chain having an amino acid sequence having at least 95% identity to an amino acid sequence of SEQ ID NO:1564. In one embodiment, the first antigen binding domain that specifically binds CD8, comprises a heavy chain having an amino acid sequence having at least 95% identity to an amino acid sequence of SEQ ID NO:1563, and a light chain having an amino acid sequence having at least 95% identity to an amino acid sequence of SEQ ID NO:1564.

In one embodiment, the first antigen binding domain that specifically binds CD8, comprises: (i) a VH comprising a VH CDR1, a VH CDR2, and a VH CDR3 having an amino acid sequence of SEQ ID NOs:1565, 1566, and 1567, respectively, and (ii) a VL comprising a VL CDR1, VL CDR2, and VL CDR3 having an amino acid sequence of SEQ ID NOs:1568, 1569, and 1570, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1571, 1572, and 1573, respectively, and (ii) a VL comprising a VL CDR1, VL CDR2, and VL CDR3 having an amino acid sequence of SEQ ID NOs:1574, 1575, and 1576, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1577, 1578, and 1579, respectively, and (ii) a VL comprising a VL CDR1, VL CDR2, and VL CDR3 having an amino acid sequence of SEQ ID NOs:1580, 1581, and 1582, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1583, 1584, and 1585, respectively, and (ii) a VL comprising a VL CDR1, VL CDR2, and VL CDR3 having an amino acid sequence of SEQ ID NOs:1586, 1587, and 1588, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1589, 1590, and 1591, respectively, and (ii) a VL comprising a VL CDR1, VL CDR2, and VL CDR3 having an amino acid sequence of SEQ ID NOs:1592, 1593, and 1594, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1595; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1596. In one embodiment, the first antigen binding domain that specifically binds CD8, comprises a VH having an amino acid sequence of SEQ ID NO:1595. In one embodiment, the first antigen binding domain that specifically binds CD8, comprises a VL having an amino acid sequence of SEQ ID NO:1596. In one embodiment, the first antigen binding domain that specifically binds CD8, comprises a VH having an amino acid sequence of SEQ ID NO:1595, and a VL having an amino acid sequence of SEQ ID NO:1596. In one embodiment, the first antigen binding domain that specifically binds CD8, comprises a heavy chain having an amino acid sequence of SEQ ID NO:1597. In one embodiment, the first antigen binding domain that specifically binds CD8, comprises a light chain having an amino acid sequence of SEQ ID NO:1598. In one embodiment, the first antigen binding domain that specifically binds CD8, comprises a heavy chain having an amino acid sequence of SEQ ID NO:1597, and a light chain having an amino acid sequence of SEQ ID NO:1598. In one embodiment, the first antigen binding domain that specifically binds CD8, comprises a VH having an amino acid sequence having at least 95% identity to an amino acid sequence of SEQ ID NO:1595. In one embodiment, the first antigen binding domain that specifically binds CD8, comprises a VL having an amino acid sequence having at least 95% identity to an amino acid sequence of SEQ ID NO:1596. In one embodiment, the first antigen binding domain that specifically binds CD8, comprises a VH having an amino acid sequence having at least 95% identity to an amino acid sequence of SEQ ID NO:1595, and a VL having an amino acid sequence having at least 95% identity to an amino acid sequence of SEQ ID NO:1596. In one embodiment, the first antigen binding domain that specifically binds CD8, comprises a heavy chain having an amino acid sequence having at least 95% identity to an amino acid sequence of SEQ ID NO:1597. In one embodiment, the first antigen binding domain that specifically binds CD8, comprises a light chain having an amino acid sequence having at least 95% identity to an amino acid sequence of SEQ ID NO:1598. In one embodiment, the first antigen binding domain that specifically binds CD8, comprises a heavy chain having an amino acid sequence having at least 95% identity to an amino acid sequence of SEQ ID NO:1597, and a light chain having an amino acid sequence having at least 95% identity to an amino acid sequence of SEQ ID NO:1598.

In one embodiment, the first antigen binding domain that specifically binds CD8, comprises: (i) a VH comprising a VH CDR1, a VH CDR2, and a VH CDR3 having an amino acid sequence of SEQ ID NOs:1599, 1600, and 1601, respectively, and (ii) a VL comprising a VL CDR1, VL CDR2, and VL CDR3 having an amino acid sequence of SEQ ID NOs:1602, 1603, and 1604, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1605, 1606, and 1607, respectively, and (ii) a VL comprising a VL CDR1, VL CDR2, and VL CDR3 having an amino acid sequence of SEQ ID NOs:1608, 1609, and 1610, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1611, 1612, and 1613, respectively, and (ii) a VL comprising a VL CDR1, VL CDR2, and VL CDR3 having an amino acid sequence of SEQ ID NOs:1614, 1615, and 1616, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1617, 1618, and 1619, respectively, and (ii) a VL comprising a VL CDR1, VL CDR2, and VL CDR3 having an amino acid sequence of SEQ ID NOs:1620, 1621, and 1622, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1623, 1624, and 1625, respectively, and (ii) a VL comprising a VL CDR1, VL CDR2, and VL CDR3 having an amino acid sequence of SEQ ID NOs:1626, 1627, and 1628, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1629; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1630. In one embodiment, the first antigen binding domain that specifically binds CD8, comprises a VH having an amino acid sequence of SEQ ID NO:1629. In one embodiment, the first antigen binding domain that specifically binds CD8, comprises a VL having an amino acid sequence of SEQ ID NO:1630. In one embodiment, the first antigen binding domain that specifically binds CD8, comprises a VH having an amino acid sequence of SEQ ID NO:1629, and a VL having an amino acid sequence of SEQ ID NO:1630. In one embodiment, the first antigen binding domain that specifically binds CD8, comprises a heavy chain having an amino acid sequence of SEQ ID NO:1631. In one embodiment, the first antigen binding domain that specifically binds CD8, comprises a light chain having an amino acid sequence of SEQ ID NO:1632. In one embodiment, the first antigen binding domain that specifically binds CD8, comprises a heavy chain having an amino acid sequence of SEQ ID NO:1631, and a light chain having an amino acid sequence of SEQ ID NO:1632. In one embodiment, the first antigen binding domain that specifically binds CD8, comprises a VH having an amino acid sequence having at least 95% identity to an amino acid sequence of SEQ ID NO:1629. In one embodiment, the first antigen binding domain that specifically binds CD8, comprises a VL having an amino acid sequence having at least 95% identity to an amino acid sequence of SEQ ID NO:1630. In one embodiment, the first antigen binding domain that specifically binds CD8, comprises a VH having an amino acid sequence having at least 95% identity to an amino acid sequence of SEQ ID NO:1629, and a VL having an amino acid sequence having at least 95% identity to an amino acid sequence of SEQ ID NO:1630. In one embodiment, the first antigen binding domain that specifically binds CD8, comprises a heavy chain having an amino acid sequence having at least 95% identity to an amino acid sequence of SEQ ID NO:1631. In one embodiment, the first antigen binding domain that specifically binds CD8, comprises a light chain having an amino acid sequence having at least 95% identity to an amino acid sequence of SEQ ID NO:1632. In one embodiment, the first antigen binding domain that specifically binds CD8, comprises a heavy chain having an amino acid sequence having at least 95% identity to an amino acid sequence of SEQ ID NO:1631, and a light chain having an amino acid sequence having at least 95% identity to an amino acid sequence of SEQ ID NO:1632.

In one embodiment, the first antigen binding domain that specifically binds CD8, comprises: (i) a VH comprising a VH CDR1, a VH CDR2, and a VH CDR3 having an amino acid sequence of SEQ ID NOs:1633, 1634, and 1635, respectively, and (ii) a VL comprising a VL CDR1, VL CDR2, and VL CDR3 having an amino acid sequence of SEQ ID NOs:1636, 1637, and 1638, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1639, 1640, and 1641, respectively, and (ii) a VL comprising a VL CDR1, VL CDR2, and VL CDR3 having an amino acid sequence of SEQ ID NOs:1642, 1643, and 1644, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1645, 1646, and 1647, respectively, and (ii) a VL comprising a VL CDR1, VL CDR2, and VL CDR3 having an amino acid sequence of SEQ ID NOs:1648, 1649, and 1650, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1651, 1652, and 1653, respectively, and (ii) a VL comprising a VL CDR1, VL CDR2, and VL CDR3 having an amino acid sequence of SEQ ID NOs:1654, 1655, and 1656, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1657, 1658, and 1659, respectively, and (ii) a VL comprising a VL CDR1, VL CDR2, and VL CDR3 having an amino acid sequence of SEQ ID NOs:1660, 1661, and 1662, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1663; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1664. In one embodiment, the first antigen binding domain that specifically binds CD8, comprises a VH having an amino acid sequence of SEQ ID NO:1663. In one embodiment, the first antigen binding domain that specifically binds CD8, comprises a VL having an amino acid sequence of SEQ ID NO:1664. In one embodiment, the first antigen binding domain that specifically binds CD8, comprises a VH having an amino acid sequence of SEQ ID NO:1663, and a VL having an amino acid sequence of SEQ ID NO:1664. In one embodiment, the first antigen binding domain that specifically binds CD8, comprises a heavy chain having an amino acid sequence of SEQ ID NO:1665. In one embodiment, the first antigen binding domain that specifically binds CD8, comprises a light chain having an amino acid sequence of SEQ ID NO:1666. In one embodiment, the first antigen binding domain that specifically binds CD8, comprises a heavy chain having an amino acid sequence of SEQ ID NO:1665, and a light chain having an amino acid sequence of SEQ ID NO:1666. In one embodiment, the first antigen binding domain that specifically binds CD8, comprises a VH having an amino acid sequence having at least 95% identity to an amino acid sequence of SEQ ID NO:1663. In one embodiment, the first antigen binding domain that specifically binds CD8, comprises a VL having an amino acid sequence having at least 95% identity to an amino acid sequence of SEQ ID NO:1664. In one embodiment, the first antigen binding domain that specifically binds CD8, comprises a VH having an amino acid sequence having at least 95% identity to an amino acid sequence of SEQ ID NO:1663, and a VL having an amino acid sequence having at least 95% identity to an amino acid sequence of SEQ ID NO:1664. In one embodiment, the first antigen binding domain that specifically binds CD8, comprises a heavy chain having an amino acid sequence having at least 95% identity to an amino acid sequence of SEQ ID NO:1665. In one embodiment, the first antigen binding domain that specifically binds CD8, comprises a light chain having an amino acid sequence having at least 95% identity to an amino acid sequence of SEQ ID NO:1666. In one embodiment, the first antigen binding domain that specifically binds CD8, comprises a heavy chain having an amino acid sequence having at least 95% identity to an amino acid sequence of SEQ ID NO:1665, and a light chain having an amino acid sequence having at least 95% identity to an amino acid sequence of SEQ ID NO:1666.

In one embodiment, the first antigen binding domain that specifically binds CD8, comprises: (i) a VH comprising a VH CDR1, a VH CDR2, and a VH CDR3 having an amino acid sequence of SEQ ID NOs:1667, 1668, and 1669, respectively, and (ii) a VL comprising a VL CDR1, VL CDR2, and VL CDR3 having an amino acid sequence of SEQ ID NOs:1670, 1671, and 1672, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1673, 1674, and 1675, respectively, and (ii) a VL comprising a VL CDR1, VL CDR2, and VL CDR3 having an amino acid sequence of SEQ ID NOs:1676, 1677, and 1678, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1679, 1680, and 1681, respectively, and (ii) a VL comprising a VL CDR1, VL CDR2, and VL CDR3 having an amino acid sequence of SEQ ID NOs:1682, 1683, and 1684, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1685, 1686, and 1687, respectively, and (ii) a VL comprising a VL CDR1, VL CDR2, and VL CDR3 having an amino acid sequence of SEQ ID NOs:1688, 1689, and 1690, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1691, 1692, and 1693, respectively, and (ii) a VL comprising a VL CDR1, VL CDR2, and VL CDR3 having an amino acid sequence of SEQ ID NOs:1694, 1695, and 1696, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1697; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1698. In one embodiment, the first antigen binding domain that specifically binds CD8, comprises a VH having an amino acid sequence of SEQ ID NO:1697. In one embodiment, the first antigen binding domain that specifically binds CD8, comprises a VL having an amino acid sequence of SEQ ID NO:1698. In one embodiment, the first antigen binding domain that specifically binds CD8, comprises a VH having an amino acid sequence of SEQ ID NO:1697, and a VL having an amino acid sequence of SEQ ID NO:1698. In one embodiment, the first antigen binding domain that specifically binds CD8, comprises a heavy chain having an amino acid sequence of SEQ ID NO:1699. In one embodiment, the first antigen binding domain that specifically binds CD8, comprises a light chain having an amino acid sequence of SEQ ID NO:1700. In one embodiment, the first antigen binding domain that specifically binds CD8, comprises a heavy chain having an amino acid sequence of SEQ ID NO:1699, and a light chain having an amino acid sequence of SEQ ID NO:1700. In one embodiment, the first antigen binding domain that specifically binds CD8, comprises a VH having an amino acid sequence having at least 95% identity to an amino acid sequence of SEQ ID NO:1697. In one embodiment, the first antigen binding domain that specifically binds CD8, comprises a VL having an amino acid sequence having at least 95% identity to an amino acid sequence of SEQ ID NO:1698. In one embodiment, the first antigen binding domain that specifically binds CD8, comprises a VH having an amino acid sequence having at least 95% identity to an amino acid sequence of SEQ ID NO:1697, and a VL having an amino acid sequence having at least 95% identity to an amino acid sequence of SEQ ID NO:1698. In one embodiment, the first antigen binding domain that specifically binds CD8, comprises a heavy chain having an amino acid sequence having at least 95% identity to an amino acid sequence of SEQ ID NO:1699. In one embodiment, the first antigen binding domain that specifically binds CD8, comprises a light chain having an amino acid sequence having at least 95% identity to an amino acid sequence of SEQ ID NO:1700. In one embodiment, the first antigen binding domain that specifically binds CD8, comprises a heavy chain having an amino acid sequence having at least 95% identity to an amino acid sequence of SEQ ID NO:1699, and a light chain having an amino acid sequence having at least 95% identity to an amino acid sequence of SEQ ID NO:1700.

In one embodiment, the first antigen binding domain that specifically binds CD8, comprises: (i) a VH comprising a VH CDR1, a VH CDR2, and a VH CDR3 having an amino acid sequence of SEQ ID NOs:1701, 1702, and 1703, respectively, and (ii) a VL comprising a VL CDR1, VL CDR2, and VL CDR3 having an amino acid sequence of SEQ ID NOs:1704, 1705, and 1706, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1707, 1708, and 1709, respectively, and (ii) a VL comprising a VL CDR1, VL CDR2, and VL CDR3 having an amino acid sequence of SEQ ID NOs:1710, 1711, and 1712, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1713, 1714, and 1715, respectively, and (ii) a VL comprising a VL CDR1, VL CDR2, and VL CDR3 having an amino acid sequence of SEQ ID NOs:1716, 1717, and 1718, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1719, 1720, and 1721, respectively, and (ii) a VL comprising a VL CDR1, VL CDR2, and VL CDR3 having an amino acid sequence of SEQ ID NOs:1722, 1723, and 1724, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1725, 1726, and 1727, respectively, and (ii) a VL comprising a VL CDR1, VL CDR2, and VL CDR3 having an amino acid sequence of SEQ ID NOs:1728, 1729, and 1730, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1731; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1732. In one embodiment, the first antigen binding domain that specifically binds CD8, comprises a VH having an amino acid sequence of SEQ ID NO:1731. In one embodiment, the first antigen binding domain that specifically binds CD8, comprises a VL having an amino acid sequence of SEQ ID NO:1732. In one embodiment, the first antigen binding domain that specifically binds CD8, comprises a VH having an amino acid sequence of SEQ ID NO:1731, and a VL having an amino acid sequence of SEQ ID NO:1732. In one embodiment, the first antigen binding domain that specifically binds CD8, comprises a heavy chain having an amino acid sequence of SEQ ID NO:1733. In one embodiment, the first antigen binding domain that specifically binds CD8, comprises a light chain having an amino acid sequence of SEQ ID NO:1734. In one embodiment, the first antigen binding domain that specifically binds CD8, comprises a heavy chain having an amino acid sequence of SEQ ID NO:1733, and a light chain having an amino acid sequence of SEQ ID NO:1734. In one embodiment, the first antigen binding domain that specifically binds CD8, comprises a VH having an amino acid sequence having at least 95% identity to an amino acid sequence of SEQ ID NO:1731. In one embodiment, the first antigen binding domain that specifically binds CD8, comprises a VL having an amino acid sequence having at least 95% identity to an amino acid sequence of SEQ ID NO:1732. In one embodiment, the first antigen binding domain that specifically binds CD8, comprises a VH having an amino acid sequence having at least 95% identity to an amino acid sequence of SEQ ID NO:1731, and a VL having an amino acid sequence having at least 95% identity to an amino acid sequence of SEQ ID NO:1732. In one embodiment, the first antigen binding domain that specifically binds CD8, comprises a heavy chain having an amino acid sequence having at least 95% identity to an amino acid sequence of SEQ ID NO:1733. In one embodiment, the first antigen binding domain that specifically binds CD8, comprises a light chain having an amino acid sequence having at least 95% identity to an amino acid sequence of SEQ ID NO:1734. In one embodiment, the first antigen binding domain that specifically binds CD8, comprises a heavy chain having an amino acid sequence having at least 95% identity to an amino acid sequence of SEQ ID NO:1733, and a light chain having an amino acid sequence having at least 95% identity to an amino acid sequence of SEQ ID NO:1734.

In one embodiment, the first antigen binding domain that specifically binds CD8, comprises: (i) a VH comprising a VH CDR1, a VH CDR2, and a VH CDR3 having an amino acid sequence of SEQ ID NOs:1735, 1736, and 1737, respectively, and (ii) a VL comprising a VL CDR1, VL CDR2, and VL CDR3 having an amino acid sequence of SEQ ID NOs:1738, 1739, and 1740, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1741, 1742, and 1743, respectively, and (ii) a VL comprising a VL CDR1, VL CDR2, and VL CDR3 having an amino acid sequence of SEQ ID NOs:1744, 1745, and 1746, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1747, 1748, and 1749, respectively, and (ii) a VL comprising a VL CDR1, VL CDR2, and VL CDR3 having an amino acid sequence of SEQ ID NOs:1750, 1751, and 1752, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1753, 1754, and 1755, respectively, and (ii) a VL comprising a VL CDR1, VL CDR2, and VL CDR3 having an amino acid sequence of SEQ ID NOs:1756, 1757, and 1758, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1759, 1760, and 1761, respectively, and (ii) a VL comprising a VL CDR1, VL CDR2, and VL CDR3 having an amino acid sequence of SEQ ID NOs:1762, 1763, and 1764, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1765; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1766. In one embodiment, the first antigen binding domain that specifically binds CD8, comprises a VH having an amino acid sequence of SEQ ID NO:1765. In one embodiment, the first antigen binding domain that specifically binds CD8, comprises a VL having an amino acid sequence of SEQ ID NO:1766. In one embodiment, the first antigen binding domain that specifically binds CD8, comprises a VH having an amino acid sequence of SEQ ID NO:1765, and a VL having an amino acid sequence of SEQ ID NO:1766. In one embodiment, the first antigen binding domain that specifically binds CD8, comprises a heavy chain having an amino acid sequence of SEQ ID NO:1767. In one embodiment, the first antigen binding domain that specifically binds CD8, comprises a light chain having an amino acid sequence of SEQ ID NO:1768. In one embodiment, the first antigen binding domain that specifically binds CD8, comprises a heavy chain having an amino acid sequence of SEQ ID NO:1767, and a light chain having an amino acid sequence of SEQ ID NO:1768. In one embodiment, the first antigen binding domain that specifically binds CD8, comprises a VH having an amino acid sequence having at least 95% identity to an amino acid sequence of SEQ ID NO:1765. In one embodiment, the first antigen binding domain that specifically binds CD8, comprises a VL having an amino acid sequence having at least 95% identity to an amino acid sequence of SEQ ID NO:1766. In one embodiment, the first antigen binding domain that specifically binds CD8, comprises a VH having an amino acid sequence having at least 95% identity to an amino acid sequence of SEQ ID NO:1765, and a VL having an amino acid sequence having at least 95% identity to an amino acid sequence of SEQ ID NO:1766. In one embodiment, the first antigen binding domain that specifically binds CD8, comprises a heavy chain having an amino acid sequence having at least 95% identity to an amino acid sequence of SEQ ID NO:1767. In one embodiment, the first antigen binding domain that specifically binds CD8, comprises a light chain having an amino acid sequence having at least 95% identity to an amino acid sequence of SEQ ID NO:1768. In one embodiment, the first antigen binding domain that specifically binds CD8, comprises a heavy chain having an amino acid sequence having at least 95% identity to an amino acid sequence of SEQ ID NO:1767, and a light chain having an amino acid sequence having at least 95% identity to an amino acid sequence of SEQ ID NO:1768.

In one embodiment, the first antigen binding domain that specifically binds CD8, comprises: (i) a VH comprising a VH CDR1, a VH CDR2, and a VH CDR3 having an amino acid sequence of SEQ ID NOs:1769, 1770, and 1771, respectively, and (ii) a VL comprising a VL CDR1, VL CDR2, and VL CDR3 having an amino acid sequence of SEQ ID NOs:1772, 1773, and 1774, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1775, 1776, and 1777, respectively, and (ii) a VL comprising a VL CDR1, VL CDR2, and VL CDR3 having an amino acid sequence of SEQ ID NOs:1778, 1779, and 1780, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1781, 1782, and 1783, respectively, and (ii) a VL comprising a VL CDR1, VL CDR2, and VL CDR3 having an amino acid sequence of SEQ ID NOs:1784, 1785, and 1786, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1787, 1788, and 1789, respectively, and (ii) a VL comprising a VL CDR1, VL CDR2, and VL CDR3 having an amino acid sequence of SEQ ID NOs:1790, 1791, and 1792, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1793, 1794, and 1795, respectively, and (ii) a VL comprising a VL CDR1, VL CDR2, and VL CDR3 having an amino acid sequence of SEQ ID NOs:1796, 1797, and 1798, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1799; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1800. In one embodiment, the first antigen binding domain that specifically binds CD8, comprises a VH having an amino acid sequence of SEQ ID NO:1799. In one embodiment, the first antigen binding domain that specifically binds CD8, comprises a VL having an amino acid sequence of SEQ ID NO:1800. In one embodiment, the first antigen binding domain that specifically binds CD8, comprises a VH having an amino acid sequence of SEQ ID NO:1799, and a VL having an amino acid sequence of SEQ ID NO:1800. In one embodiment, the first antigen binding domain that specifically binds CD8, comprises a heavy chain having an amino acid sequence of SEQ ID NO:1801. In one embodiment, the first antigen binding domain that specifically binds CD8, comprises a light chain having an amino acid sequence of SEQ ID NO:1802. In one embodiment, the first antigen binding domain that specifically binds CD8, comprises a heavy chain having an amino acid sequence of SEQ ID NO:1801, and a light chain having an amino acid sequence of SEQ ID NO:1802. In one embodiment, the first antigen binding domain that specifically binds CD8, comprises a VH having an amino acid sequence having at least 95% identity to an amino acid sequence of SEQ ID NO:1799. In one embodiment, the first antigen binding domain that specifically binds CD8, comprises a VL having an amino acid sequence having at least 95% identity to an amino acid sequence of SEQ ID NO:1800. In one embodiment, the first antigen binding domain that specifically binds CD8, comprises a VH having an amino acid sequence having at least 95% identity to an amino acid sequence of SEQ ID NO:1799, and a VL having an amino acid sequence having at least 95% identity to an amino acid sequence of SEQ ID NO:1800. In one embodiment, the first antigen binding domain that specifically binds CD8, comprises a heavy chain having an amino acid sequence having at least 95% identity to an amino acid sequence of SEQ ID NO:1801. In one embodiment, the first antigen binding domain that specifically binds CD8, comprises a light chain having an amino acid sequence having at least 95% identity to an amino acid sequence of SEQ ID NO:1802. In one embodiment, the first antigen binding domain that specifically binds CD8, comprises a heavy chain having an amino acid sequence having at least 95% identity to an amino acid sequence of SEQ ID NO:1801, and a light chain having an amino acid sequence having at least 95% identity to an amino acid sequence of SEQ ID NO:1802.

In one embodiment, the first antigen binding domain that specifically binds CD8, comprises: (i) a VH comprising a VH CDR1, a VH CDR2, and a VH CDR3 having an amino acid sequence of SEQ ID NOs:1803, 1804, and 1805, respectively, and (ii) a VL comprising a VL CDR1, VL CDR2, and VL CDR3 having an amino acid sequence of SEQ ID NOs:1806, 1807, and 1808, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1809, 1810, and 1811, respectively, and (ii) a VL comprising a VL CDR1, VL CDR2, and VL CDR3 having an amino acid sequence of SEQ ID NOs:1812, 1813, and 1814, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1815, 1816, and 1817, respectively, and (ii) a VL comprising a VL CDR1, VL CDR2, and VL CDR3 having an amino acid sequence of SEQ ID NOs:1818, 1819, and 1820, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1821, 1822, and 1823, respectively, and (ii) a VL comprising a VL CDR1, VL CDR2, and VL CDR3 having an amino acid sequence of SEQ ID NOs:1824, 1825, and 1826, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1827, 1828, and 1829, respectively, and (ii) a VL comprising a VL CDR1, VL CDR2, and VL CDR3 having an amino acid sequence of SEQ ID NOs:1830, 1831, and 1832, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1833; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1834. In one embodiment, the first antigen binding domain that specifically binds CD8, comprises a VH having an amino acid sequence of SEQ ID NO:1833. In one embodiment, the first antigen binding domain that specifically binds CD8, comprises a VL having an amino acid sequence of SEQ ID NO:1834. In one embodiment, the first antigen binding domain that specifically binds CD8, comprises a VH having an amino acid sequence of SEQ ID NO:1833, and a VL having an amino acid sequence of SEQ ID NO:1834. In one embodiment, the first antigen binding domain that specifically binds CD8, comprises a heavy chain having an amino acid sequence of SEQ ID NO:1835. In one embodiment, the first antigen binding domain that specifically binds CD8, comprises a light chain having an amino acid sequence of SEQ ID NO:1836. In one embodiment, the first antigen binding domain that specifically binds CD8, comprises a heavy chain having an amino acid sequence of SEQ ID NO:1835, and a light chain having an amino acid sequence of SEQ ID NO:1836. In one embodiment, the first antigen binding domain that specifically binds CD8, comprises a VH having an amino acid sequence having at least 95% identity to an amino acid sequence of SEQ ID NO:1833. In one embodiment, the first antigen binding domain that specifically binds CD8, comprises a VL having an amino acid sequence having at least 95% identity to an amino acid sequence of SEQ ID NO:1834. In one embodiment, the first antigen binding domain that specifically binds CD8, comprises a VH having an amino acid sequence having at least 95% identity to an amino acid sequence of SEQ ID NO:1833, and a VL having an amino acid sequence having at least 95% identity to an amino acid sequence of SEQ ID NO:1834. In one embodiment, the first antigen binding domain that specifically binds CD8, comprises a heavy chain having an amino acid sequence having at least 95% identity to an amino acid sequence of SEQ ID NO:1835. In one embodiment, the first antigen binding domain that specifically binds CD8, comprises a light chain having an amino acid sequence having at least 95% identity to an amino acid sequence of SEQ ID NO:1836. In one embodiment, the first antigen binding domain that specifically binds CD8, comprises a heavy chain having an amino acid sequence having at least 95% identity to an amino acid sequence of SEQ ID NO:1835, and a light chain having an amino acid sequence having at least 95% identity to an amino acid sequence of SEQ ID NO:1836.

In one embodiment, the first antigen binding domain that specifically binds CD8, comprises: (i) a VH comprising a VH CDR1, a VH CDR2, and a VH CDR3 having an amino acid sequence of SEQ ID NOs:1837, 1838, and 1839, respectively, and (ii) a VL comprising a VL CDR1, VL CDR2, and VL CDR3 having an amino acid sequence of SEQ ID NOs:1840, 1841, and 1842, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1843, 1844, and 1845, respectively, and (ii) a VL comprising a VL CDR1, VL CDR2, and VL CDR3 having an amino acid sequence of SEQ ID NOs:1846, 1847, and 1848, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1849, 1850, and 1851, respectively, and (ii) a VL comprising a VL CDR1, VL CDR2, and VL CDR3 having an amino acid sequence of SEQ ID NOs:1852, 1853, and 1854, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1855, 1856, and 1857, respectively, and (ii) a VL comprising a VL CDR1, VL CDR2, and VL CDR3 having an amino acid sequence of SEQ ID NOs:1858, 1859, and 1860, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1861, 1862, and 1863, respectively, and (ii) a VL comprising a VL CDR1, VL CDR2, and VL CDR3 having an amino acid sequence of SEQ ID NOs:1864, 1865, and 1866, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1867; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1868. In one embodiment, the first antigen binding domain that specifically binds CD8, comprises a VH having an amino acid sequence of SEQ ID NO:1867. In one embodiment, the first antigen binding domain that specifically binds CD8, comprises a VL having an amino acid sequence of SEQ ID NO:1868. In one embodiment, the first antigen binding domain that specifically binds CD8, comprises a VH having an amino acid sequence of SEQ ID NO:1867, and a VL having an amino acid sequence of SEQ ID NO:1868. In one embodiment, the first antigen binding domain that specifically binds CD8, comprises a heavy chain having an amino acid sequence of SEQ ID NO:1869. In one embodiment, the first antigen binding domain that specifically binds CD8, comprises a light chain having an amino acid sequence of SEQ ID NO:1870. In one embodiment, the first antigen binding domain that specifically binds CD8, comprises a heavy chain having an amino acid sequence of SEQ ID NO:1869, and a light chain having an amino acid sequence of SEQ ID NO:1870. In one embodiment, the first antigen binding domain that specifically binds CD8, comprises a VH having an amino acid sequence having at least 95% identity to an amino acid sequence of SEQ ID NO:1867. In one embodiment, the first antigen binding domain that specifically binds CD8, comprises a VL having an amino acid sequence having at least 95% identity to an amino acid sequence of SEQ ID NO:1868. In one embodiment, the first antigen binding domain that specifically binds CD8, comprises a VH having an amino acid sequence having at least 95% identity to an amino acid sequence of SEQ ID NO:1867, and a VL having an amino acid sequence having at least 95% identity to an amino acid sequence of SEQ ID NO:1868. In one embodiment, the first antigen binding domain that specifically binds CD8, comprises a heavy chain having an amino acid sequence having at least 95% identity to an amino acid sequence of SEQ ID NO:1869. In one embodiment, the first antigen binding domain that specifically binds CD8, comprises a light chain having an amino acid sequence having at least 95% identity to an amino acid sequence of SEQ ID NO:1870. In one embodiment, the first antigen binding domain that specifically binds CD8, comprises a heavy chain having an amino acid sequence having at least 95% identity to an amino acid sequence of SEQ ID NO:1869, and a light chain having an amino acid sequence having at least 95% identity to an amino acid sequence of SEQ ID NO:1870.

In one embodiment, the first antigen binding domain that specifically binds CD8, comprises: (i) a VH comprising a VH CDR1, a VH CDR2, and a VH CDR3 having an amino acid sequence of SEQ ID NOs:1871, 1872, and 1873, respectively, and (ii) a VL comprising a VL CDR1, VL CDR2, and VL CDR3 having an amino acid sequence of SEQ ID NOs:1874, 1875, and 1876, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1877, 1878, and 1879, respectively, and (ii) a VL comprising a VL CDR1, VL CDR2, and VL CDR3 having an amino acid sequence of SEQ ID NOs:1880, 1881, and 1882, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1883, 1884, and 1885, respectively, and (ii) a VL comprising a VL CDR1, VL CDR2, and VL CDR3 having an amino acid sequence of SEQ ID NOs:1886, 1887, and 1888, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1889, 1890, and 1891, respectively, and (ii) a VL comprising a VL CDR1, VL CDR2, and VL CDR3 having an amino acid sequence of SEQ ID NOs:1892, 1893, and 1894, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1895, 1896, and 1897, respectively, and (ii) a VL comprising a VL CDR1, VL CDR2, and VL CDR3 having an amino acid sequence of SEQ ID NOs:1898, 1899, and 1900, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1901; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1902. In one embodiment, the first antigen binding domain that specifically binds CD8, comprises a VH having an amino acid sequence of SEQ ID NO:1901. In one embodiment, the first antigen binding domain that specifically binds CD8, comprises a VL having an amino acid sequence of SEQ ID NO:1902. In one embodiment, the first antigen binding domain that specifically binds CD8, comprises a VH having an amino acid sequence of SEQ ID NO:1901, and a VL having an amino acid sequence of SEQ ID NO:1902. In one embodiment, the first antigen binding domain that specifically binds CD8, comprises a heavy chain having an amino acid sequence of SEQ ID NO:1903. In one embodiment, the first antigen binding domain that specifically binds CD8, comprises a light chain having an amino acid sequence of SEQ ID NO:1904. In one embodiment, the first antigen binding domain that specifically binds CD8, comprises a heavy chain having an amino acid sequence of SEQ ID NO:1903, and a light chain having an amino acid sequence of SEQ ID NO:1904. In one embodiment, the first antigen binding domain that specifically binds CD8, comprises a VH having an amino acid sequence having at least 95% identity to an amino acid sequence of SEQ ID NO:1901. In one embodiment, the first antigen binding domain that specifically binds CD8, comprises a VL having an amino acid sequence having at least 95% identity to an amino acid sequence of SEQ ID NO:1902. In one embodiment, the first antigen binding domain that specifically binds CD8, comprises a VH having an amino acid sequence having at least 95% identity to an amino acid sequence of SEQ ID NO:1901, and a VL having an amino acid sequence having at least 95% identity to an amino acid sequence of SEQ ID NO:1902. In one embodiment, the first antigen binding domain that specifically binds CD8, comprises a heavy chain having an amino acid sequence having at least 95% identity to an amino acid sequence of SEQ ID NO:1903. In one embodiment, the first antigen binding domain that specifically binds CD8, comprises a light chain having an amino acid sequence having at least 95% identity to an amino acid sequence of SEQ ID NO:1904. In one embodiment, the first antigen binding domain that specifically binds CD8, comprises a heavy chain having an amino acid sequence having at least 95% identity to an amino acid sequence of SEQ ID NO:1903, and a light chain having an amino acid sequence having at least 95% identity to an amino acid sequence of SEQ ID NO:1904.

In one embodiment, the first antigen binding domain that specifically binds CD8, comprises: (i) a VH comprising a VH CDR1, a VH CDR2, and a VH CDR3 having an amino acid sequence of SEQ ID NOs:1905, 1906, and 1907, respectively, and (ii) a VL comprising a VL CDR1, VL CDR2, and VL CDR3 having an amino acid sequence of SEQ ID NOs:1908, 1909, and 1910, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1911, 1912, and 1913, respectively, and (ii) a VL comprising a VL CDR1, VL CDR2, and VL CDR3 having an amino acid sequence of SEQ ID NOs:1914, 1915, and 1916, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1917, 1918, and 1919, respectively, and (ii) a VL comprising a VL CDR1, VL CDR2, and VL CDR3 having an amino acid sequence of SEQ ID NOs:1920, 1921, and 1922, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1923, 1924, and 1925, respectively, and (ii) a VL comprising a VL CDR1, VL CDR2, and VL CDR3 having an amino acid sequence of SEQ ID NOs:1926, 1927, and 1928, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1929, 1930, and 1931, respectively, and (ii) a VL comprising a VL CDR1, VL CDR2, and VL CDR3 having an amino acid sequence of SEQ ID NOs:1932, 1933, and 1934, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1935; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1936. In one embodiment, the first antigen binding domain that specifically binds CD8, comprises a VH having an amino acid sequence of SEQ ID NO:1935. In one embodiment, the first antigen binding domain that specifically binds CD8, comprises a VL having an amino acid sequence of SEQ ID NO:1936. In one embodiment, the first antigen binding domain that specifically binds CD8, comprises a VH having an amino acid sequence of SEQ ID NO:1935, and a VL having an amino acid sequence of SEQ ID NO:1936. In one embodiment, the first antigen binding domain that specifically binds CD8, comprises a heavy chain having an amino acid sequence of SEQ ID NO:1937. In one embodiment, the first antigen binding domain that specifically binds CD8, comprises a light chain having an amino acid sequence of SEQ ID NO:1938. In one embodiment, the first antigen binding domain that specifically binds CD8, comprises a heavy chain having an amino acid sequence of SEQ ID NO:1937, and a light chain having an amino acid sequence of SEQ ID NO:1938. In one embodiment, the first antigen binding domain that specifically binds CD8, comprises a VH having an amino acid sequence having at least 95% identity to an amino acid sequence of SEQ ID NO:1935. In one embodiment, the first antigen binding domain that specifically binds CD8, comprises a VL having an amino acid sequence having at least 95% identity to an amino acid sequence of SEQ ID NO:1936. In one embodiment, the first antigen binding domain that specifically binds CD8, comprises a VH having an amino acid sequence having at least 95% identity to an amino acid sequence of SEQ ID NO:1935, and a VL having an amino acid sequence having at least 95% identity to an amino acid sequence of SEQ ID NO:1936. In one embodiment, the first antigen binding domain that specifically binds CD8, comprises a heavy chain having an amino acid sequence having at least 95% identity to an amino acid sequence of SEQ ID NO:1937. In one embodiment, the first antigen binding domain that specifically binds CD8, comprises a light chain having an amino acid sequence having at least 95% identity to an amino acid sequence of SEQ ID NO:1938. In one embodiment, the first antigen binding domain that specifically binds CD8, comprises a heavy chain having an amino acid sequence having at least 95% identity to an amino acid sequence of SEQ ID NO:1937, and a light chain having an amino acid sequence having at least 95% identity to an amino acid sequence of SEQ ID NO:1938.

In one embodiment, the first antigen binding domain that specifically binds CD8, comprises: (i) a VH comprising a VH CDR1, a VH CDR2, and a VH CDR3 having an amino acid sequence of SEQ ID NOs:1939, 1940, and 1941, respectively, and (ii) a VL comprising a VL CDR1, VL CDR2, and VL CDR3 having an amino acid sequence of SEQ ID NOs:1942, 1943, and 1944, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1945, 1946, and 1947, respectively, and (ii) a VL comprising a VL CDR1, VL CDR2, and VL CDR3 having an amino acid sequence of SEQ ID NOs:1948, 1949, and 1950, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1951, 1952, and 1953, respectively, and (ii) a VL comprising a VL CDR1, VL CDR2, and VL CDR3 having an amino acid sequence of SEQ ID NOs:1954, 1955, and 1956, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1957, 1958, and 1959, respectively, and (ii) a VL comprising a VL CDR1, VL CDR2, and VL CDR3 having an amino acid sequence of SEQ ID NOs:1960, 1961, and 1962, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1963, 1964, and 1965, respectively, and (ii) a VL comprising a VL CDR1, VL CDR2, and VL CDR3 having an amino acid sequence of SEQ ID NOs:1966, 1967, and 1968, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:1969; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:1970. In one embodiment, the first antigen binding domain that specifically binds CD8, comprises a VH having an amino acid sequence of SEQ ID NO:1969. In one embodiment, the first antigen binding domain that specifically binds CD8, comprises a VL having an amino acid sequence of SEQ ID NO:1970. In one embodiment, the first antigen binding domain that specifically binds CD8, comprises a VH having an amino acid sequence of SEQ ID NO:1969, and a VL having an amino acid sequence of SEQ ID NO:1970. In one embodiment, the first antigen binding domain that specifically binds CD8, comprises a heavy chain having an amino acid sequence of SEQ ID NO:1971. In one embodiment, the first antigen binding domain that specifically binds CD8, comprises a light chain having an amino acid sequence of SEQ ID NO:1972. In one embodiment, the first antigen binding domain that specifically binds CD8, comprises a heavy chain having an amino acid sequence of SEQ ID NO:1971, and a light chain having an amino acid sequence of SEQ ID NO:1972. In one embodiment, the first antigen binding domain that specifically binds CD8, comprises a VH having an amino acid sequence having at least 95% identity to an amino acid sequence of SEQ ID NO:1969. In one embodiment, the first antigen binding domain that specifically binds CD8, comprises a VL having an amino acid sequence having at least 95% identity to an amino acid sequence of SEQ ID NO:1970. In one embodiment, the first antigen binding domain that specifically binds CD8, comprises a VH having an amino acid sequence having at least 95% identity to an amino acid sequence of SEQ ID NO:1969, and a VL having an amino acid sequence having at least 95% identity to an amino acid sequence of SEQ ID NO:1970. In one embodiment, the first antigen binding domain that specifically binds CD8, comprises a heavy chain having an amino acid sequence having at least 95% identity to an amino acid sequence of SEQ ID NO:1971. In one embodiment, the first antigen binding domain that specifically binds CD8, comprises a light chain having an amino acid sequence having at least 95% identity to an amino acid sequence of SEQ ID NO:1972. In one embodiment, the first antigen binding domain that specifically binds CD8, comprises a heavy chain having an amino acid sequence having at least 95% identity to an amino acid sequence of SEQ ID NO:1971, and a light chain having an amino acid sequence having at least 95% identity to an amino acid sequence of SEQ ID NO:1972.

In one embodiment, the first antigen binding domain that specifically binds CD8, comprises: (i) a VH comprising a VH CDR1, a VH CDR2, and a VH CDR3 having an amino acid sequence of SEQ ID NOs:1973, 1974, and 1975, respectively, and (ii) a VL comprising a VL CDR1, VL CDR2, and VL CDR3 having an amino acid sequence of SEQ ID NOs:1976, 1977, and 1978, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1979, 1980, and 1981, respectively, and (ii) a VL comprising a VL CDR1, VL CDR2, and VL CDR3 having an amino acid sequence of SEQ ID NOs:1982, 1983, and 1984, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1985, 1986, and 1987, respectively, and (ii) a VL comprising a VL CDR1, VL CDR2, and VL CDR3 having an amino acid sequence of SEQ ID NOs:1988, 1989, and 1990, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1991, 1992, and 1993, respectively, and (ii) a VL comprising a VL CDR1, VL CDR2, and VL CDR3 having an amino acid sequence of SEQ ID NOs:1994, 1995, and 1996, respectively. In one embodiment, the first antigen binding domain that specifically binds CD8, comprises: (i) a VH comprising a VH CDR1, a VH CDR2, and a VH CDR3 having an amino acid sequence of SEQ ID NOs:1997, 1998, and 1999, respectively, and (ii) a VL comprising a VL CDR1, VL CDR2, and VL CDR3 having an amino acid sequence of SEQ ID NOs:2000, 2001, and 2002, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2003; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2004. In one embodiment, the first antigen binding domain that specifically binds CD8, comprises a VH having an amino acid sequence of SEQ ID NO:2003. In one embodiment, the first antigen binding domain that specifically binds CD8, comprises a VL having an amino acid sequence of SEQ ID NO:2004. In one embodiment, the first antigen binding domain that specifically binds CD8, comprises a VH having an amino acid sequence of SEQ ID NO:2003, and a VL having an amino acid sequence of SEQ ID NO:2004. In one embodiment, the first antigen binding domain that specifically binds CD8, comprises a heavy chain having an amino acid sequence of SEQ ID NO:2005. In one embodiment, the first antigen binding domain that specifically binds CD8, comprises a light chain having an amino acid sequence of SEQ ID NO:2006. In one embodiment, the first antigen binding domain that specifically binds CD8, comprises a heavy chain having an amino acid sequence of SEQ ID NO:2005, and a light chain having an amino acid sequence of SEQ ID NO:2006. In one embodiment, the first antigen binding domain that specifically binds CD8, comprises a VH having an amino acid sequence having at least 95% identity to an amino acid sequence of SEQ ID NO:2003. In one embodiment, the first antigen binding domain that specifically binds CD8, comprises a VL having an amino acid sequence having at least 95% identity to an amino acid sequence of SEQ ID NO:2004. In one embodiment, the first antigen binding domain that specifically binds CD8, comprises a VH having an amino acid sequence having at least 95% identity to an amino acid sequence of SEQ ID NO:2003, and a VL having an amino acid sequence having at least 95% identity to an amino acid sequence of SEQ ID NO:2004. In one embodiment, the first antigen binding domain that specifically binds CD8, comprises a heavy chain having an amino acid sequence having at least 95% identity to an amino acid sequence of SEQ ID NO:2005. In one embodiment, the first antigen binding domain that specifically binds CD8, comprises a light chain having an amino acid sequence having at least 95% identity to an amino acid sequence of SEQ ID NO:2006. In one embodiment, the first antigen binding domain that specifically binds CD8, comprises a heavy chain having an amino acid sequence having at least 95% identity to an amino acid sequence of SEQ ID NO:2005, and a light chain having an amino acid sequence having at least 95% identity to an amino acid sequence of SEQ ID NO:2006.

In one embodiment, the first antigen binding domain that specifically binds CD8, comprises: (i) a VH comprising a VH CDR1, a VH CDR2, and a VH CDR3 having an amino acid sequence of SEQ ID NOs:2007, 2008, and 2009, respectively, and (ii) a VL comprising a VL CDR1, VL CDR2, and VL CDR3 having an amino acid sequence of SEQ ID NOs:2010, 2011, and 2012, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2013, 2014, and 2015, respectively, and (ii) a VL comprising a VL CDR1, VL CDR2, and VL CDR3 having an amino acid sequence of SEQ ID NOs:2016, 2017, and 2018, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2019, 2020, and 2021, respectively, and (ii) a VL comprising a VL CDR1, VL CDR2, and VL CDR3 having an amino acid sequence of SEQ ID NOs:2022, 2023, and 2024, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2025, 2026, and 2027, respectively, and (ii) a VL comprising a VL CDR1, VL CDR2, and VL CDR3 having an amino acid sequence of SEQ ID NOs:2028, 2029, and 2030, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2031, 2032, and 2033, respectively, and (ii) a VL comprising a VL CDR1, VL CDR2, and VL CDR3 having an amino acid sequence of SEQ ID NOs:2034, 2035, and 2036, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2037; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2038. In one embodiment, the first antigen binding domain that specifically binds CD8, comprises a VH having an amino acid sequence of SEQ ID NO:2037. In one embodiment, the first antigen binding domain that specifically binds CD8, comprises a VL having an amino acid sequence of SEQ ID NO:2038. In one embodiment, the first antigen binding domain that specifically binds CD8, comprises a VH having an amino acid sequence of SEQ ID NO:2037, and a VL having an amino acid sequence of SEQ ID NO:2038. In one embodiment, the first antigen binding domain that specifically binds CD8, comprises a heavy chain having an amino acid sequence of SEQ ID NO:2039. In one embodiment, the first antigen binding domain that specifically binds CD8, comprises a light chain having an amino acid sequence of SEQ ID NO:2040. In one embodiment, the first antigen binding domain that specifically binds CD8, comprises a heavy chain having an amino acid sequence of SEQ ID NO:2039, and a light chain having an amino acid sequence of SEQ ID NO:2040. In one embodiment, the first antigen binding domain that specifically binds CD8, comprises a VH having an amino acid sequence having at least 95% identity to an amino acid sequence of SEQ ID NO:2037. In one embodiment, the first antigen binding domain that specifically binds CD8, comprises a VL having an amino acid sequence having at least 95% identity to an amino acid sequence of SEQ ID NO:2038. In one embodiment, the first antigen binding domain that specifically binds CD8, comprises a VH having an amino acid sequence having at least 95% identity to an amino acid sequence of SEQ ID NO:2037, and a VL having an amino acid sequence having at least 95% identity to an amino acid sequence of SEQ ID NO:2038. In one embodiment, the first antigen binding domain that specifically binds CD8, comprises a heavy chain having an amino acid sequence having at least 95% identity to an amino acid sequence of SEQ ID NO:2039. In one embodiment, the first antigen binding domain that specifically binds CD8, comprises a light chain having an amino acid sequence having at least 95% identity to an amino acid sequence of SEQ ID NO:2040. In one embodiment, the first antigen binding domain that specifically binds CD8, comprises a heavy chain having an amino acid sequence having at least 95% identity to an amino acid sequence of SEQ ID NO:2039, and a light chain having an amino acid sequence having at least 95% identity to an amino acid sequence of SEQ ID NO:2040.

In one embodiment, the first antigen binding domain that specifically binds CD8, comprises: (i) a VH comprising a VH CDR1, a VH CDR2, and a VH CDR3 having an amino acid sequence of SEQ ID NOs:2041, 2042, and 2043, respectively, and (ii) a VL comprising a VL CDR1, VL CDR2, and VL CDR3 having an amino acid sequence of SEQ ID NOs:2044, 2045, and 2046, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2047, 2048, and 2049, respectively, and (ii) a VL comprising a VL CDR1, VL CDR2, and VL CDR3 having an amino acid sequence of SEQ ID NOs:2050, 2051, and 2052, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2053, 2054, and 2055, respectively, and (ii) a VL comprising a VL CDR1, VL CDR2, and VL CDR3 having an amino acid sequence of SEQ ID NOs:2056, 2057, and 2058, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2059, 2060, and 2061, respectively, and (ii) a VL comprising a VL CDR1, VL CDR2, and VL CDR3 having an amino acid sequence of SEQ ID NOs:2062, 2063, and 2064, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2065, 2066, and 2067, respectively, and (ii) a VL comprising a VL CDR1, VL CDR2, and VL CDR3 having an amino acid sequence of SEQ ID NOs:2068, 2069, and 2070, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2071; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2072. In one embodiment, the first antigen binding domain that specifically binds CD8, comprises a VH having an amino acid sequence of SEQ ID NO:2071. In one embodiment, the first antigen binding domain that specifically binds CD8, comprises a VL having an amino acid sequence of SEQ ID NO:2072. In one embodiment, the first antigen binding domain that specifically binds CD8, comprises a VH having an amino acid sequence of SEQ ID NO:2071, and a VL having an amino acid sequence of SEQ ID NO:2072. In one embodiment, the first antigen binding domain that specifically binds CD8, comprises a heavy chain having an amino acid sequence of SEQ ID NO:2073. In one embodiment, the first antigen binding domain that specifically binds CD8, comprises a light chain having an amino acid sequence of SEQ ID NO:2074. In one embodiment, the first antigen binding domain that specifically binds CD8, comprises a heavy chain having an amino acid sequence of SEQ ID NO:2073, and a light chain having an amino acid sequence of SEQ ID NO:2074. In one embodiment, the first antigen binding domain that specifically binds CD8, comprises a VH having an amino acid sequence having at least 95% identity to an amino acid sequence of SEQ ID NO:2071. In one embodiment, the first antigen binding domain that specifically binds CD8, comprises a VL having an amino acid sequence having at least 95% identity to an amino acid sequence of SEQ ID NO:2072. In one embodiment, the first antigen binding domain that specifically binds CD8, comprises a VH having an amino acid sequence having at least 95% identity to an amino acid sequence of SEQ ID NO:2071, and a VL having an amino acid sequence having at least 95% identity to an amino acid sequence of SEQ ID NO:2072. In one embodiment, the first antigen binding domain that specifically binds CD8, comprises a heavy chain having an amino acid sequence having at least 95% identity to an amino acid sequence of SEQ ID NO:2073. In one embodiment, the first antigen binding domain that specifically binds CD8, comprises a light chain having an amino acid sequence having at least 95% identity to an amino acid sequence of SEQ ID NO:2074. In one embodiment, the first antigen binding domain that specifically binds CD8, comprises a heavy chain having an amino acid sequence having at least 95% identity to an amino acid sequence of SEQ ID NO:2073, and a light chain having an amino acid sequence having at least 95% identity to an amino acid sequence of SEQ ID NO:2074.

In one embodiment, the first antigen binding domain that specifically binds CD8, comprises: (i) a VH comprising a VH CDR1, a VH CDR2, and a VH CDR3 having an amino acid sequence of SEQ ID NOs:2075, 2076, and 2077, respectively, and (ii) a VL comprising a VL CDR1, VL CDR2, and VL CDR3 having an amino acid sequence of SEQ ID NOs:2078, 2079, and 2080, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2081, 2082, and 2083, respectively, and (ii) a VL comprising a VL CDR1, VL CDR2, and VL CDR3 having an amino acid sequence of SEQ ID NOs:2084, 2085, and 2086, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2087, 2088, and 2089, respectively, and (ii) a VL comprising a VL CDR1, VL CDR2, and VL CDR3 having an amino acid sequence of SEQ ID NOs:2090, 2091, and 2092, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2093, 2094, and 2095, respectively, and (ii) a VL comprising a VL CDR1, VL CDR2, and VL CDR3 having an amino acid sequence of SEQ ID NOs:2096, 2097, and 2098, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2099, 2100, and 2101, respectively, and (ii) a VL comprising a VL CDR1, VL CDR2, and VL CDR3 having an amino acid sequence of SEQ ID NOs:2102, 2103, and 2104, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2105; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2106. In one embodiment, the first antigen binding domain that specifically binds CD8, comprises a VH having an amino acid sequence of SEQ ID NO:2105. In one embodiment, the first antigen binding domain that specifically binds CD8, comprises a VL having an amino acid sequence of SEQ ID NO:2106. In one embodiment, the first antigen binding domain that specifically binds CD8, comprises a VH having an amino acid sequence of SEQ ID NO:2105, and a VL having an amino acid sequence of SEQ ID NO:2106. In one embodiment, the first antigen binding domain that specifically binds CD8, comprises a heavy chain having an amino acid sequence of SEQ ID NO:2107. In one embodiment, the first antigen binding domain that specifically binds CD8, comprises a light chain having an amino acid sequence of SEQ ID NO:2108. In one embodiment, the first antigen binding domain that specifically binds CD8, comprises a heavy chain having an amino acid sequence of SEQ ID NO:2107, and a light chain having an amino acid sequence of SEQ ID NO:2108. In one embodiment, the first antigen binding domain that specifically binds CD8, comprises a VH having an amino acid sequence having at least 95% identity to an amino acid sequence of SEQ ID NO:2105. In one embodiment, the first antigen binding domain that specifically binds CD8, comprises a VL having an amino acid sequence having at least 95% identity to an amino acid sequence of SEQ ID NO:2106. In one embodiment, the first antigen binding domain that specifically binds CD8, comprises a VH having an amino acid sequence having at least 95% identity to an amino acid sequence of SEQ ID NO:2105, and a VL having an amino acid sequence having at least 95% identity to an amino acid sequence of SEQ ID NO:2106. In one embodiment, the first antigen binding domain that specifically binds CD8, comprises a heavy chain having an amino acid sequence having at least 95% identity to an amino acid sequence of SEQ ID NO:2107. In one embodiment, the first antigen binding domain that specifically binds CD8, comprises a light chain having an amino acid sequence having at least 95% identity to an amino acid sequence of SEQ ID NO:2108. In one embodiment, the first antigen binding domain that specifically binds CD8, comprises a heavy chain having an amino acid sequence having at least 95% identity to an amino acid sequence of SEQ ID NO:2107, and a light chain having an amino acid sequence having at least 95% identity to an amino acid sequence of SEQ ID NO:2108.

In one embodiment, the first antigen binding domain that specifically binds CD8, comprises: (i) a VH comprising a VH CDR1, a VH CDR2, and a VH CDR3 having an amino acid sequence of SEQ ID NOs:2109, 2110, and 2111, respectively, and (ii) a VL comprising a VL CDR1, VL CDR2, and VL CDR3 having an amino acid sequence of SEQ ID NOs:2112, 2113, and 2114, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2115, 2116, and 2117, respectively, and (ii) a VL comprising a VL CDR1, VL CDR2, and VL CDR3 having an amino acid sequence of SEQ ID NOs:2118, 2119, and 2120, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2121, 2122, and 2123, respectively, and (ii) a VL comprising a VL CDR1, VL CDR2, and VL CDR3 having an amino acid sequence of SEQ ID NOs:2124, 2125, and 2126, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2127, 2128, and 2129, respectively, and (ii) a VL comprising a VL CDR1, VL CDR2, and VL CDR3 having an amino acid sequence of SEQ ID NOs:2130, 2131, and 2132, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2133, 2134, and 2135, respectively, and (ii) a VL comprising a VL CDR1, VL CDR2, and VL CDR3 having an amino acid sequence of SEQ ID NOs:2136, 2137, and 2138, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2139; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2140. In one embodiment, the first antigen binding domain that specifically binds CD8, comprises a VH having an amino acid sequence of SEQ ID NO:2139. In one embodiment, the first antigen binding domain that specifically binds CD8, comprises a VL having an amino acid sequence of SEQ ID NO:2140. In one embodiment, the first antigen binding domain that specifically binds CD8, comprises a VH having an amino acid sequence of SEQ ID NO:2139, and a VL having an amino acid sequence of SEQ ID NO:2140. In one embodiment, the first antigen binding domain that specifically binds CD8, comprises a heavy chain having an amino acid sequence of SEQ ID NO:2141. In one embodiment, the first antigen binding domain that specifically binds CD8, comprises a light chain having an amino acid sequence of SEQ ID NO:2142. In one embodiment, the first antigen binding domain that specifically binds CD8, comprises a heavy chain having an amino acid sequence of SEQ ID NO:2141, and a light chain having an amino acid sequence of SEQ ID NO:2142. In one embodiment, the first antigen binding domain that specifically binds CD8, comprises a VH having an amino acid sequence having at least 95% identity to an amino acid sequence of SEQ ID NO:2139. In one embodiment, the first antigen binding domain that specifically binds CD8, comprises a VL having an amino acid sequence having at least 95% identity to an amino acid sequence of SEQ ID NO:2140. In one embodiment, the first antigen binding domain that specifically binds CD8, comprises a VH having an amino acid sequence having at least 95% identity to an amino acid sequence of SEQ ID NO:2139, and a VL having an amino acid sequence having at least 95% identity to an amino acid sequence of SEQ ID NO:2140. In one embodiment, the first antigen binding domain that specifically binds CD8, comprises a heavy chain having an amino acid sequence having at least 95% identity to an amino acid sequence of SEQ ID NO:2141. In one embodiment, the first antigen binding domain that specifically binds CD8, comprises a light chain having an amino acid sequence having at least 95% identity to an amino acid sequence of SEQ ID NO:2142. In one embodiment, the first antigen binding domain that specifically binds CD8, comprises a heavy chain having an amino acid sequence having at least 95% identity to an amino acid sequence of SEQ ID NO:2141, and a light chain having an amino acid sequence having at least 95% identity to an amino acid sequence of SEQ ID NO:2142.

In one embodiment, the first antigen binding domain that specifically binds CD8, comprises: (i) a VH comprising a VH CDR1, a VH CDR2, and a VH CDR3 having an amino acid sequence of SEQ ID NOs:2143, 2144, and 2145, respectively, and (ii) a VL comprising a VL CDR1, VL CDR2, and VL CDR3 having an amino acid sequence of SEQ ID NOs:2146, 2147, and 2148, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2149, 2150, and 2151, respectively, and (ii) a VL comprising a VL CDR1, VL CDR2, and VL CDR3 having an amino acid sequence of SEQ ID NOs:2152, 2153, and 2154, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2155, 2156, and 2157, respectively, and (ii) a VL comprising a VL CDR1, VL CDR2, and VL CDR3 having an amino acid sequence of SEQ ID NOs:2158, 2159, and 2160, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2161, 2162, and 2163, respectively, and (ii) a VL comprising a VL CDR1, VL CDR2, and VL CDR3 having an amino acid sequence of SEQ ID NOs:2164, 2165, and 2166, respectively. In one embodiment, the first antigen binding domain that specifically binds CD8, comprises: (i) a VH comprising a VH CDR1, a VH CDR2, and a VH CDR3 having an amino acid sequence of SEQ ID NOs:2167, 2168, and 2169, respectively, and (ii) a VL comprising a VL CDR1, VL CDR2, and VL CDR3 having an amino acid sequence of SEQ ID NOs:2170, 2171, and 2172, respectively. In one embodiment, the first antigen binding domain that specifically binds CD8, comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2173; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:2174. In one embodiment, the first antigen binding domain that specifically binds CD8, comprises a VH having an amino acid sequence of SEQ ID NO:2173. In one embodiment, the first antigen binding domain that specifically binds CD8, comprises a VL having an amino acid sequence of SEQ ID NO:2174. In one embodiment, the first antigen binding domain that specifically binds CD8, comprises a VH having an amino acid sequence of SEQ ID NO:2173, and a VL having an amino acid sequence of SEQ ID NO:2174. In one embodiment, the first antigen binding domain that specifically binds CD8, comprises a heavy chain having an amino acid sequence of SEQ ID NO:2175. In one embodiment, the first antigen binding domain that specifically binds CD8, comprises a light chain having an amino acid sequence of SEQ ID NO:2176. In one embodiment, the first antigen binding domain that specifically binds CD8, comprises a heavy chain having an amino acid sequence of SEQ ID NO:2175, and a light chain having an amino acid sequence of SEQ ID NO:2176. In one embodiment, the first antigen binding domain that specifically binds CD8, comprises a VH having an amino acid sequence having at least 95% identity to an amino acid sequence of SEQ ID NO:2173. In one embodiment, the first antigen binding domain that specifically binds CD8, comprises a VL having an amino acid sequence having at least 95% identity to an amino acid sequence of SEQ ID NO:2174. In one embodiment, the first antigen binding domain that specifically binds CD8, comprises a VH having an amino acid sequence having at least 95% identity to an amino acid sequence of SEQ ID NO:2173, and a VL having an amino acid sequence having at least 95% identity to an amino acid sequence of SEQ ID NO:2174. In one embodiment, the first antigen binding domain that specifically binds CD8, comprises a heavy chain having an amino acid sequence having at least 95% identity to an amino acid sequence of SEQ ID NO:2175. In one embodiment, the first antigen binding domain that specifically binds CD8, comprises a light chain having an amino acid sequence having at least 95% identity to an amino acid sequence of SEQ ID NO:2176. In one embodiment, the first antigen binding domain that specifically binds CD8, comprises a heavy chain having an amino acid sequence having at least 95% identity to an amino acid sequence of SEQ ID NO:2175, and a light chain having an amino acid sequence having at least 95% identity to an amino acid sequence of SEQ ID NO:2176.

In some embodiments, the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296.

In some embodiments, the second antigen binding domain that specifically binds CD3 comprises the VH of SEQ ID NO: 2297 and the VL of SEQ ID NO: 2298.

The disclosure also provides an isolated molecule, comprising: a first antigen binding domain and a second antigen binding domain, wherein the first antigen binding domain specifically binds CD8 and the second antigen binding domain specifically binds CD3. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds a third antigen. It is contemplated that an isolated molecule provided herein can comprise a first antigen binding domain that specifically binds CD8 provided herein, a second antigen binding domain that specifically binds CD3 provided herein, and a third antigen binding domain that specifically binds a third antigen provided herein.

In some embodiments, the third antigen comprises an antigen expressed by an undesired cell.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8 and is unable to activate or recruit CD8⁺ CTLs in the absence of co-engagement of CD3 and CD8. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3 and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein.

The disclosure also provides an isolated multispecific antibody, comprising: a first antigen binding domain and a second antigen binding domain, wherein the first antigen binding domain specifically binds CD8 and the second antigen binding domain specifically binds CD3. Exemplary first antigen binding domains and second antigen binding domains are provided herein. It is contemplated that an isolated multispecific antibody provided herein can comprise any first antigen binding domain specifically binds CD8 provided herein, and any second antigen binding domain specifically binds CD3 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds a third antigen. Exemplary first antigen binding domains and second antigen binding domains are provided herein. It is contemplated that an isolated multispecific antibody provided herein can comprise a first antigen binding domain that specifically binds CD8 provided herein, a second antigen binding domain that specifically binds CD3 provided herein, and a third antigen binding domain that specifically binds a third antigen provided herein.

In some embodiments, the third antigen comprises an antigen expressed by an undesired cell.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell. Exemplary first antigen binding domains and second antigen binding domains are provided herein. It is contemplated that an isolated multispecific antibody provided herein can comprise a first antigen binding domain that specifically binds CD8 provided herein, a second antigen binding domain that specifically binds CD3 provided herein, and a third antigen binding domain that specifically binds a third antigen provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8. Exemplary first antigen binding domains and second antigen binding domains are provided herein. It is contemplated that an isolated multispecific antibody provided herein can comprise a first antigen binding domain that specifically binds CD8 provided herein, a second antigen binding domain that specifically binds CD3 provided herein, and a third antigen binding domain that specifically binds a third antigen provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8 and wherein the isolated multispecific antibody is unable to activate or recruit CD8⁺ CTLs in the absence of co-engagement of CD3 and CD8. Exemplary first antigen binding domains and second antigen binding domains are provided herein. It is contemplated that an isolated multispecific antibody provided herein can comprise a first antigen binding domain that specifically binds CD8 provided herein, a second antigen binding domain that specifically binds CD3 provided herein, and a third antigen binding domain that specifically binds a third antigen provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8. Exemplary first antigen binding domains and second antigen binding domains are provided herein. It is contemplated that an isolated multispecific antibody provided herein can comprise a first antigen binding domain that specifically binds CD8 provided herein, a second antigen binding domain that specifically binds CD3 provided herein, and a third antigen binding domain that specifically binds a third antigen provided herein.

The disclosure also provides an isolated molecule, comprising: a first antigen binding domain and a second antigen binding domain, wherein the first antigen binding domain specifically binds CD8 and the second antigen binding domain specifically binds CD3, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds a third antigen, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8 and is unable to activate or recruit CD8⁺ CTLs in the absence of co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3 and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296. In certain embodiments, the isolated molecule comprises a first antigen binding domain that specifically binds CD8 provided herein.

The disclosure also provides an isolated multispecific antibody, comprising: a first antigen binding domain and a second antigen binding domain, wherein the first antigen binding domain specifically binds CD8 and the second antigen binding domain specifically binds CD3, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds a third antigen, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8 and wherein the isolated multispecific antibody is unable to activate or recruit CD8⁺ CTLs in the absence of co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296. In certain embodiments, the isolated multispecific antibody comprises a first antigen binding domain that specifically binds CD8 provided herein.

The disclosure also provides an isolated molecule, comprising: a first antigen binding domain and a second antigen binding domain, wherein the first antigen binding domain specifically binds CD8 and the second antigen binding domain specifically binds CD3, wherein the second antigen binding domain that specifically binds CD3 comprises the VH of SEQ ID NO: 2297 and the VL of SEQ ID NO: 2298. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds a third antigen, wherein the second antigen binding domain that specifically binds CD3 comprises the VH of SEQ ID NO: 2297 and the VL of SEQ ID NO: 2298. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the second antigen binding domain that specifically binds CD3 comprises the VH of SEQ ID NO: 2297 and the VL of SEQ ID NO: 2298. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8, wherein the second antigen binding domain that specifically binds CD3 comprises the VH of SEQ ID NO: 2297 and the VL of SEQ ID NO: 2298. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8 and is unable to activate or recruit CD8⁺ CTLs in the absence of co-engagement of CD3 and CD8, wherein the second antigen binding domain that specifically binds CD3 comprises the VH of SEQ ID NO: 2297 and the VL of SEQ ID NO: 2298. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3 and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the second antigen binding domain that specifically binds CD3 comprises the VH of SEQ ID NO: 2297 and the VL of SEQ ID NO: 2298. In certain embodiments, the isolated molecule comprises a first antigen binding domain that specifically binds CD8 provided herein.

The disclosure also provides an isolated multispecific antibody, comprising: a first antigen binding domain and a second antigen binding domain, wherein the first antigen binding domain specifically binds CD8 and the second antigen binding domain specifically binds CD3, wherein the second antigen binding domain that specifically binds CD3 comprises the VH of SEQ ID NO: 2297 and the VL of SEQ ID NO: 2298. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds a third antigen, wherein the second antigen binding domain that specifically binds CD3 comprises the VH of SEQ ID NO: 2297 and the VL of SEQ ID NO: 2298. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the second antigen binding domain that specifically binds CD3 comprises the VH of SEQ ID NO: 2297 and the VL of SEQ ID NO: 2298. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8, wherein the second antigen binding domain that specifically binds CD3 comprises the VH of SEQ ID NO: 2297 and the VL of SEQ ID NO: 2298. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8 and wherein the isolated multispecific antibody is unable to activate or recruit CD8⁺ CTLs in the absence of co-engagement of CD3 and CD8, wherein the second antigen binding domain that specifically binds CD3 comprises the VH of SEQ ID NO: 2297 and the VL of SEQ ID NO: 2298. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the second antigen binding domain that specifically binds CD3 comprises the VH of SEQ ID NO: 2297 and the VL of SEQ ID NO: 2298. In certain embodiments, the isolated multispecific antibody comprises a first antigen binding domain that specifically binds CD8 provided herein.

The disclosure also provides an isolated molecule, comprising: a first antigen binding domain and a second antigen binding domain, wherein the first antigen binding domain specifically binds CD8 and the second antigen binding domain specifically binds CD3, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds a third antigen, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8 and is unable to activate or recruit CD8⁺ CTLs in the absence of co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3 and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

The disclosure also provides an isolated multispecific antibody, comprising: a first antigen binding domain and a second antigen binding domain, wherein the first antigen binding domain specifically binds CD8 and the second antigen binding domain specifically binds CD3, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds a third antigen, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8 and wherein the isolated multispecific antibody is unable to activate or recruit CD8⁺ CTLs in the absence of co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

The disclosure also provides an isolated molecule, comprising: a first antigen binding domain and a second antigen binding domain, wherein the first antigen binding domain specifically binds CD8 and the second antigen binding domain specifically binds CD3, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds a third antigen, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8 and is unable to activate or recruit CD8⁺ CTLs in the absence of co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3 and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296.

The disclosure also provides an isolated multispecific antibody, comprising: a first antigen binding domain and a second antigen binding domain, wherein the first antigen binding domain specifically binds CD8 and the second antigen binding domain specifically binds CD3, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds a third antigen, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8 and wherein the isolated multispecific antibody is unable to activate or recruit CD8⁺ CTLs in the absence of co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296.

The disclosure also provides an isolated molecule, comprising: a first antigen binding domain, a second antigen binding domain and a third antigen binding domain, wherein the first antigen binding domain specifically binds CD8, the second antigen binding domain specifically binds a TCR complex, and the third antigen binding domain specifically binds an antigen expressed by an undesired cell, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds a TCR complex provided herein. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds a TCR complex, and the third antigen binding domain specifically binds an antigen expressed by an undesired cell, wherein the isolated molecule activates or recruits CD8⁺ CTLs upon co-engagement of the TCR complex and CD8, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds a TCR complex provided herein. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds a TCR complex, and the third antigen binding domain specifically binds an antigen expressed by an undesired cell, wherein the isolated molecule activates or recruits CD8⁺ CTLs upon co-engagement of the TCR complex and CD8 and is unable to activate or recruit CD8⁺ CTLs in the absence of co-engagement of the TCR complex and CD8, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds a TCR complex provided herein. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds a TCR complex, and the third antigen binding domain specifically binds an antigen expressed by an undesired cell, wherein the first antigen binding domain specifically binds CD8 and the second antigen binding domain specifically binds the TCR with affinities that result in activation or recruitment of CD8⁺ CTLs only upon co-engagement of the TCR complex and CD8, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds a TCR complex provided herein. In certain embodiments, the isolated molecule comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds a TCR complex, and the third antigen binding domain specifically binds an antigen expressed by an undesired cell, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds a TCR complex provided herein. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds a TCR complex, and the third antigen binding domain specifically binds an antigen expressed by an undesired cell, wherein the isolated multispecific antibody activates or recruits CD8⁺ CTLs upon co-engagement of the TCR complex and CD8, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds a TCR complex provided herein. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds a TCR complex, and the third antigen binding domain specifically binds an antigen expressed by an undesired cell, wherein the isolated multispecific antibody activates or recruits CD8⁺ CTLs upon co-engagement of the TCR complex and CD8 and wherein the isolated multispecific antibody is unable to activate or recruit CD8⁺ CTLs in the absence of co-engagement of the TCR complex and CD8, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds a TCR complex provided herein. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds a TCR complex, and the third antigen binding domain specifically binds an antigen expressed by an undesired cell, wherein the first antigen binding domain specifically binds CD8 and the second antigen binding domain specifically binds the TCR complex with affinities that result in activation or recruitment of CD8⁺ CTLs only upon co-engagement of the TCR complex and CD8, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds a TCR complex provided herein. In certain embodiments, the isolated multispecific antibody comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8 and is unable to activate or recruit CD8⁺ CTLs in the absence of co-engagement of CD3 and CD8, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3 and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated molecule comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8 and wherein the isolated multispecific antibody is unable to activate or recruit CD8⁺ CTLs in the absence of co-engagement of CD3 and CD8, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, and wherein the antigen expressed by the undesired cell is BCMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated multispecific antibody comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8 and is unable to activate or recruit CD8⁺ CTLs in the absence of co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3 and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA. In certain embodiments, the isolated molecule comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8 and wherein the isolated multispecific antibody is unable to activate or recruit CD8⁺ CTLs in the absence of co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA. In certain embodiments, the isolated multispecific antibody comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the second antigen binding domain that specifically binds CD3 comprises the VH of SEQ ID NO: 2297 and the VL of SEQ ID NO: 2298, and wherein the antigen expressed by the undesired cell is BCMA. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8, wherein the second antigen binding domain that specifically binds CD3 comprises the VH of SEQ ID NO: 2297 and the VL of SEQ ID NO: 2298, and wherein the antigen expressed by the undesired cell is BCMA. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8 and is unable to activate or recruit CD8⁺ CTLs in the absence of co-engagement of CD3 and CD8, wherein the second antigen binding domain that specifically binds CD3 comprises the VH of SEQ ID NO: 2297 and the VL of SEQ ID NO: 2298, and wherein the antigen expressed by the undesired cell is BCMA. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3 and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the second antigen binding domain that specifically binds CD3 comprises the VH of SEQ ID NO: 2297 and the VL of SEQ ID NO: 2298, and wherein the antigen expressed by the undesired cell is BCMA. In certain embodiments, the isolated molecule comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the second antigen binding domain that specifically binds CD3 comprises the VH of SEQ ID NO: 2297 and the VL of SEQ ID NO: 2298, and wherein the antigen expressed by the undesired cell is BCMA. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8, wherein the second antigen binding domain that specifically binds CD3 comprises the VH of SEQ ID NO: 2297 and the VL of SEQ ID NO: 2298, and wherein the antigen expressed by the undesired cell is BCMA. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8 and wherein the isolated multispecific antibody is unable to activate or recruit CD8⁺ CTLs in the absence of co-engagement of CD3 and CD8, wherein the second antigen binding domain that specifically binds CD3 comprises the VH of SEQ ID NO: 2297 and the VL of SEQ ID NO: 2298, and wherein the antigen expressed by the undesired cell is BCMA. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the second antigen binding domain that specifically binds CD3 comprises the VH of SEQ ID NO: 2297 and the VL of SEQ ID NO: 2298, and wherein the antigen expressed by the undesired cell is BCMA. In certain embodiments, the isolated multispecific antibody comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is BCMA.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is BCMA.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8 and is unable to activate or recruit CD8⁺ CTLs in the absence of co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is BCMA.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3 and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is BCMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is BCMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is BCMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8 and wherein the isolated multispecific antibody is unable to activate or recruit CD8⁺ CTLs in the absence of co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is BCMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is BCMA.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8 and is unable to activate or recruit CD8⁺ CTLs in the absence of co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3 and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8 and wherein the isolated multispecific antibody is unable to activate or recruit CD8⁺ CTLs in the absence of co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is BCMA.

The disclosure also provides an isolated molecule, comprising: a first antigen binding domain, a second antigen binding domain and a third antigen binding domain, wherein the first antigen binding domain specifically binds CD8, the second antigen binding domain specifically binds a TCR complex, and the third antigen binding domain specifically binds an antigen expressed by an undesired cell, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds aTCR complex provided herein. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds a TCR complex, and the third antigen binding domain specifically binds an antigen expressed by an undesired cell, wherein the isolated molecule activates or recruits CD8⁺ CTLs upon co-engagement of the TCR complex and CD8, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds aTCR complex provided herein. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds a TCR complex, and the third antigen binding domain specifically binds an antigen expressed by an undesired cell, wherein the isolated molecule activates or recruits CD8⁺ CTLs upon co-engagement of the TCR complex and CD8 and is unable to activate or recruit CD8⁺ CTLs in the absence of co-engagement of the TCR complex and CD8, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds aTCR complex provided herein. In certain embodiments, the isolated molecule antibody comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds a TCR complex, and the third antigen binding domain specifically binds an antigen expressed by an undesired cell, wherein the first antigen binding domain specifically binds CD8 and the second antigen binding domain specifically binds the TCR with affinities that result in activation or recruitment of CD8⁺ CTLs only upon co-engagement of the TCR complex and CD8, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds aTCR complex provided herein. In certain embodiments, the isolated molecule comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds a TCR complex, and the third antigen binding domain specifically binds an antigen expressed by an undesired cell, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds aTCR complex provided herein. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds a TCR complex, and the third antigen binding domain specifically binds an antigen expressed by an undesired cell, wherein the isolated multispecific antibody activates or recruits CD8⁺ CTLs upon co-engagement of the TCR complex and CD8, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds aTCR complex provided herein. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds a TCR complex, and the third antigen binding domain specifically binds an antigen expressed by an undesired cell, wherein the isolated multispecific antibody activates or recruits CD8⁺ CTLs upon co-engagement of the TCR complex and CD8 and wherein the isolated multispecific antibody is unable to activate or recruit CD8⁺ CTLs in the absence of co-engagement of the TCR complex and CD8, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds aTCR complex provided herein. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds a TCR complex, and the third antigen binding domain specifically binds an antigen expressed by an undesired cell, wherein the first antigen binding domain specifically binds CD8 and the second antigen binding domain specifically binds the TCR complex with affinities that result in activation or recruitment of CD8⁺ CTLs only upon co-engagement of the TCR complex and CD8, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds aTCR complex provided herein. In certain embodiments, the isolated multispecific antibody comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8 and is unable to activate or recruit CD8⁺ CTLs in the absence of co-engagement of CD3 and CD8, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3 and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated molecule comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8 and wherein the isolated multispecific antibody is unable to activate or recruit CD8⁺ CTLs in the absence of co-engagement of CD3 and CD8, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, and wherein the antigen expressed by the undesired cell is PSMA. Exemplary first antigen binding domains and second antigen binding domains are provided herein. It is contemplated that an isolated molecule provided herein can comprise any first antigen binding domain specifically binds CD8 provided herein, and any second antigen binding domain specifically binds CD3 provided herein. In certain embodiments, the isolated multispecific antibody comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8 and is unable to activate or recruit CD8⁺ CTLs in the absence of co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3 and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA. In certain embodiments, the isolated molecule comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8 and wherein the isolated multispecific antibody is unable to activate or recruit CD8⁺ CTLs in the absence of co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA. In certain embodiments, the isolated multispecific antibody comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the second antigen binding domain that specifically binds CD3 comprises the VH of SEQ ID NO: 2297 and the VL of SEQ ID NO: 2298, and wherein the antigen expressed by the undesired cell is PSMA. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8, wherein the second antigen binding domain that specifically binds CD3 comprises the VH of SEQ ID NO: 2297 and the VL of SEQ ID NO: 2298, and wherein the antigen expressed by the undesired cell is PSMA. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8 and is unable to activate or recruit CD8⁺ CTLs in the absence of co-engagement of CD3 and CD8, wherein the second antigen binding domain that specifically binds CD3 comprises the VH of SEQ ID NO: 2297 and the VL of SEQ ID NO: 2298, and wherein the antigen expressed by the undesired cell is PSMA. In certain embodiments, the isolated molecule comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3 and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the second antigen binding domain that specifically binds CD3 comprises the VH of SEQ ID NO: 2297 and the VL of SEQ ID NO: 2298, and wherein the antigen expressed by the undesired cell is PSMA. In certain embodiments, the isolated molecule comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the second antigen binding domain that specifically binds CD3 comprises the VH of SEQ ID NO: 2297 and the VL of SEQ ID NO: 2298, and wherein the antigen expressed by the undesired cell is PSMA. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8, wherein the second antigen binding domain that specifically binds CD3 comprises the VH of SEQ ID NO: 2297 and the VL of SEQ ID NO: 2298, and wherein the antigen expressed by the undesired cell is PSMA. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8 and wherein the isolated multispecific antibody is unable to activate or recruit CD8⁺ CTLs in the absence of co-engagement of CD3 and CD8, wherein the second antigen binding domain that specifically binds CD3 comprises the VH of SEQ ID NO: 2297 and the VL of SEQ ID NO: 2298, and wherein the antigen expressed by the undesired cell is PSMA. In certain embodiments, the isolated multispecific antibody comprises a first antigen binding domain that specifically binds CD8 provided herein.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the second antigen binding domain that specifically binds CD3 comprises the VH of SEQ ID NO: 2297 and the VL of SEQ ID NO: 2298, and wherein the antigen expressed by the undesired cell is PSMA. In certain embodiments, the isolated multispecific antibody comprises a first antigen binding domain that specifically binds CD8 provided herein.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is PSMA.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is PSMA.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8 and is unable to activate or recruit CD8⁺ CTLs in the absence of co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is PSMA.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3 and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is PSMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is PSMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is PSMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8 and wherein the isolated multispecific antibody is unable to activate or recruit CD8⁺ CTLs in the absence of co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is PSMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and wherein the antigen expressed by the undesired cell is PSMA.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3, and the third antigen binding domain specifically binds an antigen expressed by an undesired cell, wherein the isolated molecule activates or recruits CD8⁺ CTLs upon co-engagement of CD3 and CD8 and is unable to activate or recruit CD8⁺ CTLs in the absence of co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA.

The disclosure also provides an isolated molecule, comprising: a first antigen binding domain, a second antigen binding domain and a third antigen binding domain, wherein the first antigen binding domain specifically binds CD8, the second antigen binding domain specifically binds CD3 and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the isolated multispecific antibody activates or recruits CD8⁺ CTLs upon co-engagement of CD3 and CD8 and wherein the isolated multispecific antibody is unable to activate or recruit CD8⁺ CTLs in the absence of co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA.

The disclosure also provides an isolated multispecific antibody, comprising: a first half molecule and a second half molecule, wherein the first half molecule comprises a first antigen binding domain and a second antigen binding domain and the second half molecule comprises a third antigen binding domain, wherein the first antigen binding domain specifically binds CD8, the second antigen binding domain specifically binds CD3, and the third antigen binding domain specifically binds an antigen expressed by an undesired cell, wherein the first antigen binding domain specifically binds CD8 and the second antigen binding domain specifically binds CD3 with affinities that result in activation or recruitment of CD8⁺ CTLs only upon co-engagement of CD3 and CD8, wherein the first antigen binding domain that specifically binds CD8 comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312, and the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296, and wherein the antigen expressed by the undesired cell is PSMA.

The isolated molecule or the isolated multispecific antibody of the disclosure may be targeted to any undesired cell via the antigen binding domain that specifically binds an antigen expressed by the undesired cell. The isolated molecule or the multispecific antibody of the disclosure may be further engineered to comprise additional antigen binding domains which may, for example, bind a second antigen expressed by the undesired cell. In some embodiments, the undesired cell is a pathogenic cell. In some embodiments, the pathogenic cell is a cancer cell, a virus infected cell, an immune cell, an inflamed cell, a damaged cells, a foreign cell, an apoptotic cell, a dysplastic cell, an immunogenic cell, a metaplastic cell or a mutant cell, or any combination thereof.

In some embodiments, the isolated molecule or the isolated multispecific antibody of the disclosure may bind an antigen that is inert in a system the antibody is used, such as a virus coat protein, such as RSV. The isolated molecule or the isolated multispecific antibody incorporating an inert arm may be used as a research tool as is known and described herein.

In some embodiments, the undesired cell is a cancer cell. In some embodiments, the cancer cell is a malignant cancer cell. In some embodiments, the cancer cell originates from a solid tumor. In some embodiments, the cancer cell originates from a hematological malignancy.

In some embodiments, the cancer cell originates from adenocarcinoma, anal cancer, basal cell carcinoma, biliary tract cancer, bladder cancer, bone cancer, breast cancer, cancer associated with infection, cancer of the adrenal gland, cancer of the endocrine system, cancer of the head or neck, cancer of the parathyroid gland, cancer of the penis, cancer of the thyroid gland, cancer of the urethra, cervical cancer, carcinoma of the breast, carcinoma of the fallopian tubes, carcinoma of the liver, carcinoma of the lung, carcinoma of the prostate, carcinoma of the renal pelvis, carcinoma of the vagina, carcinoma of the vulva, choriocarcinoma, clear cell carcinoma, colon cancer, colon carcinoma, colorectal cancer, connective tissue cancer, cutaneous or intraocular malignant melanoma, environmentally induced cancer, gastric cancer, gastrointestinal cancer, glioma, glioblastoma, endometrial cancer, epithelial cancer, esophageal cancer, eye cancer, larynx cancer, liver cancer, hepatocellular carcinoma, hormone refractory prostate adenocarcinoma, Kaposi's sarcoma, kidney cancer, lung cancer gastro-esophageal cancer, melanoma, mesothelioma, Merkel cell cancer, neuroblastoma, non-small cell lung cancer (NSCLC), osteosarcoma, ovarian cancer, pancreatic cancer, prostate cancer, rectal cancer, renal cell carcinoma, retinoblastoma rhabdomyosarcoma, squamous cell cancer, soft tissue sarcoma, solid tumors of childhood, spinal axis tumor, stomach cancer, testicular cancer, thyroid cancer, uterine cancer, urothelial carcinoma or sarcomas, or any combination thereof.

In some embodiments, the cancer cell originates from B cell malignancies. In some embodiments, the cancer cell originates from T cell malignancies. In some embodiments, the cancer cell originates from NK cell malignancies. In some embodiments, the cancer cell originates from acute lymphoblastic leukemia, acute myeloid leukemia, anaplastic large-cell lymphoma, Burkitt's lymphoma, chronic lymphocytic leukemia, chronic myeloid leukemia, diffuse large B-cell lymphoma, dendritic cell neoplasm, follicular lymphoma, hairy cell leukemia, Hodgkin's lymphoma, leukemia, B cell leukemia, T cell leukemia, light chain amyloidosis, lymphoma, B cell lymphoma, NK cell lymphoma, T cell lymphoma, mantle-cell lymphoma, marginal zone B-cell lymphoma, monoclonal gammopathy of undetermined significance, mucosa-associated lymphatic tissue lymphoma, multiple myeloma, myelodysplastic syndrome, non-Hodgkin's lymphoma, plasma cell leukemia, precursor B-cell lymphoblastic leukemia, smoldering multiple myeloma or Waldenstrom's macroglobulinemia.

In some embodiments, the undesired cell is an infected cell. In some embodiments, the undesired cell is infected with bacteria, virus, fungi, protozoa, parasite or prion. In some embodiments, the undesired cell is a bacterial infected cell. In some embodiments, the undesired cell is a virus infected cell.

In some embodiments, the virus infected cell is infected with adenovirus, arboviral encephalitis virus, coronavirus, coxsackie virus, cytomegalovirus (CMV), dengue virus, echovirus, Epstein Barr virus, flaviviruses, human immunodeficiency virus (HIV), hepatitis A virus, hepatitis B virus, hepatitis C virus, herpes virus, HTLV virus, influenza virus, JC virus, measles virus, molluscum virus, mumps virus, papillomavirus, parvovirus, poliovirus, rabies virus, respiratory syncytial virus, rhinovirus, rotavirus, rubella virus or vaccinia virus.

In some embodiments, the undesired cell is an immune cell. In some embodiments, the undesired cell is an activated immune cell. In some embodiments, the immune cell is a CD4⁺ cell. CD4⁺ expressing cells include Th1, Th2, Th9, Th17, T-follicular helper (Tfh), Treg, central memory (Tcm), effector memory (Tem), tissue resident memory (Trm), T peripheral helper (Tph) and memory stem cells (Tscm). In some embodiments, the immune cell is a Th1 cell. In some embodiments, the immune cell is a Th2 cell. In some embodiments, the immune cell is a Th9 cell. In some embodiments, the immune cell is a Th17 cell. In some embodiments, the immune cells is a Treg cell. In some embodiments, the immune cells is an antigen-presenting cell. In some embodiments, the immune cells is a macrophage. In some embodiments, the immune cells is a M1 macrophage. In some embodiments, the immune cells is a M2 macrophage. In some embodiments, the immune cells is a dendritic cell. In some embodiments, the immune cell is a B cell. In some embodiments, the immune cell is a natural killer (NK) cell. In some embodiments, the immune cells is a B regulatory (Breg) cell. In some embodiments, the immune cell is a myeloid derived suppressor cell (MDSC) cell. In some embodiments, the immune cell is a neutrophil. In some embodiments, the immune cell is a mast cell. In some embodiments, the immune cell is a CD8⁺ T cell that lacks expression of CD3. In some embodiments, the immune cell is an activated T cell. In some embodiments, the immune cell is a granulocyte.

In some embodiments, the undesired cell is a platelet. In some embodiments, the undesired cell is an endothelial cell. In some embodiments, the undesired cell is an epithelial cell.

In some embodiments, the undesired cell is a cell that contributes to pathogenesis of an immune-mediated disease, such as an inflammatory disease, an autoimmune disease or any condition resulting in tissue damage destruction, or any combination thereof.

In some embodiments, the undesired cell is a B cell that contributes to pathogenesis of multiple sclerosis, type 1 diabetes or rheumatoid arthritis.

In some embodiments, the undesired cell is a γδ T cell that contributes to pathogenesis of an autoimmune disease, such as rheumatoid arthritis or systemic lupus erythematosus (SLE).

In some embodiments, the undesired cell is a PD-1⁺CD4⁺ T cell, such as Tfh or Tph cell, that promotes B cell responses and antibody production and contribute to autoimmune diseases driven by autoantibody production, including rheumatoid arthritis, systemic lupus erythematosus, and Sjogren's Syndrome (see e.g., US2019/0298850).

In some embodiments, the antigen expressed by an undesired cell is a tumor-associated antigens (TAAs) or tumor-specific antigens (TSAs). In some embodiments, the antigen expressed by an undesired cell comprises mesothelin, alpha-fetoprotein (ALP), BAGE, BCR-ABL, beta-catenin, beta-HCG, BrE3-antigen, BCA225, BCMA, BTAA, CA125, CA195, CA242, CA-50, CAM43, CAMEL, CAP-1, carbonic anhydrase IX, CA19-9, CA72-4, CAM 17.1, CASP-8, CCCL19, CCCL21, CD1, CD 1a, CD2, CD4, CD5, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD47, CD52, CD54, CD55, CD59, CD64, CD66a-e, CD67, CD68, CD70, CD70L, CD74, CD79a, CD79b, CD80, CD83, CD95, CD123, CD126, CD132, CD133, CD138, CD147, CD154, CDC27, CDK4, CDK4m, CDKN2A, CO-029, CTLA4, CXCR4, CXCR7, CXCL12, HIF-1a, colon-specific antigen-p (CSAp), CEACAM5) CEACAM6, c-Met, DAM, E2A-PRL, EGFR, EGFRvIII, EGP-1, EGP-2, ELF2-M, Ep-CAM, FGF, FGF-5, Flt-1, Flt-3, folate receptor, G250 antigen, Ga733VEpCAM, GAGE, gplOO, GRO-b, H4-RET, HLA-DR, HM1.24, human chorionic gonadotropin (HCG) HER2, HER3, HMGB-1, HIF-1, HSP70-2M, HST-2, HTgp-175, la, IGF-1R, IFN-g, IFN-α, IFN-b, IFN-1, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KLK2, KSA, KS-1-antigen, KS1-4, LAGE-1a, Le-Y, LDR/FUT, M344, MA-50, macrophage migration inhibitory factor (MIF), MAGE, MAGE-1, MAGE-3, MAGE-4, MAGE-5, MAGE-6, MART-1, MART-2, TRAG-3, MCP-1, MIP-1A, MIP-1B, MIF, MG7-Ag, MOV18, MUC1, MUC2, MUC3, MUC4, MUC5ac, MUC13, MUC16, MUM-1/2, MUM-3, MYL-RAR, NB/70K, Nm23H1, NuMA, NCA66, NCA95, NCA90, NY-ESO-1, p15, p16, p185erbB2, p180erbB3, PAM4 antigen, pancreatic cancer mucin, PD-1, PD-L1, PD-L2, PI5, placental growth factor, p53, PLAGL2, Pmel17 prostatic acid phosphatase, PSA, PRAME, PSMA, PlGF, ILGF, ILGF-1R, IL-6, IL-25, RCAS1, RS5, RAGE, RANTES, Ras, T101, SAGE, S100, SLAMF7, survivin, survivin-2B, SDDCAG16, TA-90\Mac2 binding protein, TAAL6, TAC, TAG-72, TLP, tenascin, TMEFF2, TRAIL receptors, TRP-1, TRP-2, TSP-180, VEGFR, ED-B fibronectin, WT-1, 17-1A-antigen, C3, C3a, C3b, C5a, C5, bcl-2, K-ras, tumor neoantigen or a viral antigen associated with cancer.

In some embodiments, the antigen expressed by an undesired cell is a viral antigen or a bacterial antigen. In some embodiments, the tumor antigen is a viral antigen derived from a virus associated with a human chronic disease or cancer (such as cervical cancer). For example, in some embodiments, the viral antigen is derived from Epstein-Barr virus (EBV), HPV antigens E6 and/or E7, hepatitis C virus (HCV), hepatitis B virus (HBV), or cytomegalovirus (CMV).

In some embodiments, the antigen expressed by an undesired cell is an antigen expressed by undesired immune cells. In some embodiments, the antigen expressed by undesired immune cells is CD19, CD20, CD38, BCMA, FcγRIIB, CD4, IL-12β2R, IL-18R, CD25, CTLA-4, CD40L, CD28, CD56, CD38, CD14, CD33, CD11c, CD123, CD66b, CD41, CD61, CD62, CD235a, CD146, CD326, CD23 or CD203c.

Exemplary cancers or tumors and specific tumor antigens associated with such tumors (but not exclusively), include acute lymphoblastic leukemia (etv6, amll, cyclophilin b), B cell lymphoma (Ig-idiotype), glioma (E-cadherin, a-catenin, b-catenin, g-catenin, p120ctn), bladder cancer (p21ras), biliary cancer (p21ras), breast cancer (MUC family, HER2/neu, c-erbB-2), cervical carcinoma (p53, p21ras), colon carcinoma (p21ras, HER2/neu, c-erbB-2, MUC family), colorectal cancer (Colorectal associated antigen (CRC)-CO17-1A/GA733, APC), choriocarcinoma (CEA), epithelial cell cancer (cyclophilin b), gastric cancer (HER2/neu, c-erbB-2, ga733 glycoprotein), hepatocellular cancer (a-fetoprotein), Hodgkins lymphoma (Imp-1, EBNA-1), lung cancer (CEA, MAGE-3, NY-ESO-1), lymphoid cell-derived leukemia (cyclophilin b), melanoma (p5 protein, gp75, oncofetal antigen, GM2 and GD2 gangliosides, Melan-A/MART-1, cdc27, MAGE-3, p21ras, gplOO), myeloma (MUC family, p21ras), non-small cell lung carcinoma (HER2/neu, c-erbB-2), nasopharyngeal cancer (Imp-1, EBNA-1), ovarian cancer (MUC family, HER2/neu, c-erbB-2), prostate cancer (KLK2, Prostate Specific Antigen (PSA) and its antigenic epitopes PSA-1, PSA-2, and PSA-3, PSMA, HER2/neu, c-erbB-2, ga733 glycoprotein, TMEFF2), renal cancer (HER2/neu, c-erbB-2), squamous cell cancers of the cervix and esophagus, testicular cancer (NY-ESO-1), T cell leukemia (HTLV-1 epitopes), and viral products or proteins, multiple myeloma (CD38, BCMA), AML (CD33, flt3), B cell malignancies (CD19, CD20, CD38), light chain amyloidosis (CD38).

Neoantigens presented on various tumor cells in the context of MHC may also be targeted using the isolated molecules or the multispecific antibodies of the disclosure. In these instances, the first antigen binding domain that specifically binds an antigen on undesired cells specifically binds a peptide/MHC complex expressed by the undesired cells. In these instances the isolated molecules or the multispecific antibodies of the disclosure may be used to target undesired cells harboring intracellular mutant, dysfunctional or foreign proteins. Exemplary neoantigens which may be targeted are disclosed for example in U.S. Ser. No. 10/155,031, US20180153975, US20190030147 and WO2017173321.

Exemplary antigens on undesired B cells comprise CD19, CD20, CD38, BCMA and FcγRII.

Exemplary antigens on undesired CD4⁺ T cells comprise CD4, IL-12β2R and IL-18R.

Exemplary antigens on undesired activated T cells comprise CD25, CTLA-4 and CD40L.

Exemplary antigens on undesired T cells comprise CD28.

Exemplary antigens on undesired NK cells comprise CD56 and CD38.

Exemplary antigens on undesired macrophages comprise CD14 and CD33.

Exemplary antigens on undesired monocytes comprise CD14 and CD33.

Exemplary antigens on undesired dendritic cells comprise CD11c and CD123.

Exemplary antigens on undesired granulocytes comprise CD66b.

Exemplary antigens on undesired platelets comprise CD41, CD61 and CD62.

Exemplary antigens on undesired erythrocytes comprise CD235a.

Exemplary antigens on undesired endothelial cells comprise CD146.

Exemplary antigens on undesired epithelial cells comprise CD326.

Exemplary antigens on undesired mast cells comprise FcεR1, CD23 and CD203c.

Exemplary antigens on undesired Tfh or Tph cells comprise PD-1.

### Methods of Making Molecules of the Disclosure

Antigen Binding Domains that Specifically Bind the TCR Complex, CD8 or an Antigen Expressed by an Undesired Cell.

The antigen binding domains that specifically bind the TCR complex, CD8 or the antigen expressed by the undesired cell may be generated using known molecular biology technologies. The various antigen binding domains may be already known domains or they may be selected de novo using known methods.

Antigen binding domains of desired specificity may be selected from a phage, mammalian or E. coli libraries expressing human immunoglobulins or portions thereof such as Fabs, single chain antibodies (scFv), unpaired or paired antibody variable regions, camelid VHH domains or non-antibody scaffolds. The libraries may be screened for binding to the desired antigen and the obtained positive clones may be further characterized, re-engineered into various antigen binding domain formats as described herein and incorporated into the isolated molecules or isolated multispecific antibodies of the disclosure.

The hybridoma method of Kohler and Milstein may be used to identify VH/VL pairs from non-human species having the desired specificity.

Antigen binding domains of desired specificity may also be generated by immunizing non-human animals and subsequently humanized. Exemplary humanization techniques including selection of human acceptor frameworks include CDR grafting (U.S. Pat. No. 5,225,539), SDR grafting (U.S. Pat. No. 6,818,749), Resurfacing (Padlan, (1991) Mol Immunol 28:489-499), Specificity Determining Residues Resurfacing (U.S. Patent Publ. No. 2010/0261620), human framework adaptation (U.S. Pat. No. 8,748,356) or superhumanization (U.S. Pat. No. 7,709,226). In these methods, CDRs or a subset of CDR residues of parental antibodies are transferred onto human frameworks that may be selected based on their overall homology to the parental frameworks, based on similarity in CDR length, or canonical structure identity, or a combination thereof.

Transgenic animals, such as mice, rat or chicken carrying human immunoglobulin (Ig) loci in their genome may be used to generate antigen binding domains of desired specificity and are described in for example U.S. Pat. No. 6,150,584, Int. Patent Publ. No. WO1999/45962, Int. Patent Publ. Nos. WO2002/066630, WO2002/43478, WO2002/043478 and WO1990/04036. The endogenous immunoglobulin loci in such animal may be disrupted or deleted, and at least one complete or partial human immunoglobulin locus may be inserted into the genome of the animal using homologous or non-homologous recombination, using transchromosomes, or using minigenes. Companies such as Regeneron (http://_www_regeneron_com), Harbour Antibodies (http://_www_harbourantibodies_com), Open Monoclonal Technology, Inc. (OMT) (http://_www_omtinc_net), KyMab (http://_www_kymab_com), Trianni (http://_www.trianni_com) and Ablexis (http://_www_ablexis_com) may be engaged to provide human antibodies directed against a selected antigen using technologies as described above.

Humanized antigen biding domains may be further optimized to improve their selectivity or affinity to a desired antigen by incorporating altered framework support residues to preserve binding affinity (backmutations) by techniques such as those described in Int. Patent Publ. Nos. WO1090/007861 and WO1992/22653, or by introducing variation at any of the CDRs for example to improve affinity of the antigen binding domain.

Preparation of antigens (e.g., the TCR complex, CD8 and an antigen expressed by an undesired cell), their expression and production of antigen binding domains of the disclosure may be performed using any suitable technique, such as recombinant protein production. The antigens may be administered to an animal in the form of purified protein, or protein mixtures including whole cells or cell or tissue extracts, or the antigen may be formed de novo in the animal's body from nucleic acids encoding said antigen or a portion thereof.

Antigens presented on MCH, either class I or class II, may be prepared as recombinant antigen/MHC complexes using known methods, such as covalently coupling the antigen (i.e., peptide) to the MHC, optionally using cleavable linkers and expressing the complex as soluble molecules in a format such peptide-β2-α2-α1-β1 chain, peptide-α1-β1-α2-β2 or peptide-α1-α2-α3 as a heterodimer with β2 macroglobulin. Linkers which are at least 15 amino acids long may be used between the antigen and the MCH. Various additional expression formats are disclosed in U.S. Pat. Nos. 5,976,551, 5,734,023, 5,820,866, 7,141,656B2, U.S. Pat. No. 6,270,772B1 and U.S. Pat. No. 7,074,905B2.

### Molecular Formats

The molecules or the multispecific antibodies of the disclosure may be engineered into any multivalent format using any known or de novo identified antigen binding domain as long as molecules or the multispecific antibodies of the disclosure comprise the first antigen binding domain that specifically binds the undesired antigen, the second antigen binding domain that specifically binds the TCR complex and the third antigen binding domain that specifically binds CD8, and through selection of the first antigen binding domain and the second antigen binding domain, activate or recruit CD8⁺ CTLs cells only upon co-engagement of the TCR complex and CD8. Exemplary formats are disclosed herein, and include molecules into which the antigen binding domains are engineered as scFv, Fab, Fv, VHH, dAb, VH, VL, Fab or as non-antibody scaffold as disclosed herein onto one or more Fc domains or fragment thereof, or optionally onto other scaffolds such as half-life extending moieties including albumin, transferrin or PEG. In the multispecific antibodies of the disclosure containing a first half molecule and a second half molecule, the second antigen binding domain that specifically binds the TCR complex and the third antigen binding domain that specifically binds CD8 may be engineered into the second half molecule and the antigen binding domain that specifically binds the antigen un undesired cells may be engineered into the first half molecule to provide spatial closeness of the second antigen binding domain and the third antigen binding domain to facilitate co-engagement. Exemplary formats that may be used (and their binding specificity) are:

Format 1:

1^{st} polypeptide: scFv(TCRcomplex)-VH(CD8)-CH1-hinge-CH2-CH3

2^{nd} polypeptide: VL(CD8)-CL

3^{rd} polypeptide: scFv(antigen on undesired cell)-Fc

Format 2:

1^{st} polypeptide: VH(CD8)-CH1-hinge-CH2-CH3

2^{nd} polypeptide: VL(CD8)-CL-scFv(TCRcomplex)

3^{rd} polypeptide: scFv(antigen on undesired cell)-Fc

Format 3:

1^{st} polypeptide: VH(CD8)-CH1-hinge-CH2-CH3-scFv(TCRcomplex)

2^{nd} polypeptide: VL(CD8)-CL

3^{rd} polypeptide: scFv(antigen on undesired cell)-Fc

Format 4:

1^{st} polypeptide: scFv(TCRcomplex)-VH(CD8)-CH1-hinge-CH2-CH3

2^{nd} polypeptide: VL(CD8)-CL

3^{rd} polypeptide: scFv(inert)-Fc

Format 5:

1^{st} polypeptide: VH(CD8)-CH1-hinge-CH2-CH3

2^{nd} polypeptide: VL(CD8)-CL-scFv(TCRcomplex)

3^{rd} polypeptide: scFv(inert)-Fc

Format 6:

1^{st} polypeptide: VH(CD8)-CH1-hinge-CH2-CH3-scFv(TCRcomplex)

2^{nd} polypeptide: VL(CD8)-CL

3^{rd} polypeptide: scFv(inert)-Fc

Fab used in the isolated molecules or in the multispecific antibodies of the disclosure may also be engineered by exchanging the VL and the VH domains for each other or exchanging the CH1 and LC domains for each other, as described in Int. Pat. Publ. No. WO2009/080251. Correct Fab pairing may also be promoted by introducing one or more amino acid substitutions in the CH1, CL, VH or VL domains of the Fab. The amino acids that are modified are typically part of the VH:VL and CH1:CL interface such that the Fab components preferentially pair with each other rather than with components of other Fabs. The amino acid substitutions may be made at the conserved framework residues of the VH/VL and CH1/CL domains. The modifications introduced in the VH and CH1 and/or VL and CL domains may be complementary to each other and may be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces. Exemplary substitutions are described in WO2014/150973 and WO2014/082179, and include a T192E substitution in the CH1 domain and S114A and N137K substitutions in the CL domain, which introduces a salt-bridge between the CH1 and CL domains (see, Golay et al., 2016, J Immunol 196:3199-211). Alternatively, the Fab domain may comprise a 143Q and 188V substitutions in the CH1 domain and 113T and 176V substitutions in the CL domain, which serves to swap hydrophobic and polar regions of contact between the CH1 and CL domain (see, Golay et al., 2016, J Immunol 196:3199-211).

Fabs may also be engineered into a single chain Fab fragment, which is a polypeptide consisting of VH-CH1-VL-CL and an optional linker between the various domains. Exemplary single chain Fab fragments that may be used in the isolated molecules or in the multispecific antibodies of the disclosure include formats in N- to C-terminal order: VH-CH1-linker-VL-CL, VL-CL-linker-VH-CH1, VH-CL-linker-VL-CH1 or VL-CH1-linker-VH-CL. The linker may be a polypeptide of at least 30 amino acids, such as between about 32 and about 50 amino acids. The single chain Fab domains may be stabilized via the natural disulfide bond between the CL domain and the CH1 domain or alternatively, via an engineered disulfide bond between the VH and the VL between following positions: VH position 44 to VL position 100, VH position n105 to VL position 43, or VH position 101 to VL position 100 (numbering according to the EU index.

scFvs may be incorporated into the isolated molecules or into the multispecific antibodies of the disclosure in either order, e.g., from N- to C-terminus in the order VH-linker-VL or VL-linker-VH. scFvs incorporated into the molecules of the disclosure may be stabilized by engineering interdomain disulfide bonds between the VH and the VL. The disulfide bond may be engineered for example between the VH position H44 and the VL position L100, between the VH position H46 and the VL position L98, between the VH position H101 and the VL position L44, between the VH position H103 and the VL position L42, or between the VH position H103 and the VL position L43 (see. e.g., Zhao et al., Int J Mol Sci 12: 1-11, 2011).

VHH domains from Camelidae family, such as camels, llamas and alpacas, as well as other single domain antibodies may also be incorporated as antigen binding domains into the isolated molecules or in the multispecific antibodies of the disclosure. The VHH domains may be further engineered at hallmark residues, such as residues 11, 37, 44, 45 and 47 (residue numbering according to Kabat) (Muyldermans, Reviews Mol Biotech 74:277-302 (2001), U.S. Pat. No. 9,156,905).

Non-antibody scaffolds may also be used as antigen binding domains and incorporated into the molecules or the multispecific antibodies of the disclosure. Such scaffolds are typically derived from repeat proteins and include ankyrin repeat proteins (DARPins), Avimers (short for avidity multimers; domain A of LDL receptor), Anticalin/Lipocalins, Kunitz domains, Affibodies, Adnexins, Affilins, Affitins (also known as Nanofitins), Knottins, Pronectins, Versabodies, Duocalins, and Fynomers and fibronectin type III (Fn3) repeat based scaffold such as Centyrins. Non-antibody scaffolds that can be used include those described in Mintz and Crea, 2013, Bioprocess International 11(2):40-48).

Additional formats that incorporate the desired multispecificity into the molecules or the multispecific antibodies of the disclosure that may be used include those described in Int. Pat. Publ. WO2019/195535. For example, a Fab, Fv, scFv or non-antibody scaffolds (e.g., non-immunoglobulin based domains) may be attached to one or two Fc domains or fragments thereof or to a light chain or fragment thereof, either N- or C-terminally, to generate trispecific molecules. Antigen binding domains may also be conjugated head-to-tail into one Fc or fragment thereof or into one light chain or fragment thereof. Additional trispecific formats that may be used are formats disclosed in WO2014/145806; WO2017/124002; Liu et al., Front Immunol. 8:38, 2017; Brinkmann & Kontermann, 2017, mAbs 9:2, 182-212; US2016/0355600; Klein et al., 2016, MAbs 8(6):1010-20; and US2017/0145116, or formats further engineered by incorporating one or more additional antigen binding domains into the formats disclosed in any of the references.

The isolated molecules or the multispecific antibodies of the disclosure comprising a first half molecule and a second half molecule, or two Fc domains or fragments thereof, may be engineered to promote preferred association of the first half molecule and the second half molecule or the two Fc domains or fragments thereof by engineering mutations into the CH3 domains which promote heterodimerization of the first half molecule and the second half molecule or the two Fc domains or fragments thereof (instead of homodimerization) Exemplary CH3 mutations that may be used in the first half molecule and in the second half molecule include technologies such as Duobody® mutations (Genmab), Knob-in-Hole mutations (Genentech), electrostatically-matched mutations (Chugai, Amgen, NovoNordisk, Oncomed), the Strand Exchange Engineered Domain body (SEEDbody) (EMD Serono), and other asymmetric mutations (e.g., Zymeworks). Duobody® mutations (Genmab) are disclosed for example in U.S. Pat. No. 9,150,663 and US2014/0303356 and include mutations F405L/K409R, wild-type/F405L_R409K, T350I_K370T_F405L/K409R, K370W/K409R, D399AFGHILMNRSTVWY/K409R, T366ADEFGHILMQVY/K409R, L368ADEGHNRSTVQ/K409AGRH, D399FHKRQ/K409AGRH, F405IKLSTVW/K409AGRH and Y407LWQ/K409AGRH. Knob-in-hole mutations are disclosed for example in WO1996/027011 and include mutations on the interface of CH3 region in which an amino acid with a small side chain (hole) is introduced into the first CH3 region and an amino acid with a large side chain (knob) is introduced into the second CH3 region, resulting in preferential interaction between the first CH3 region and the second CH3 region. Exemplary CH3 region mutations forming a knob and a hole are T366Y/F405A, T366W/F405W, F405W/Y407A, T394W/Y407T, T394S/Y407A, T366W/T394S, F405W/T394S and T366W/T366S_L368A_Y407V. Heterodimer formation may be promoted by using electrostatic interactions by substituting positively charged residues on the first CH3 region and negatively charged residues on the second CH3 region as described in US2010/0015133, US2009/0182127, US2010/028637 or US2011/0123532. Other asymmetric mutations that may be used to promote heavy chain heterodimerization are L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849. SEEDbody mutations involve substituting select IgG residues with IgA residues to promote heterodimerization as described in US20070287170. Other exemplary mutations that may be used are R409D_K370E/D399K_E357K, S354C_T366W/Y349C_T366S_L368A_Y407V, Y349C_T366W/S354C_T366S_L368A_Y407V, T366K/L351D, L351K/Y349E, L351K/Y349D, L351K/L368E, L351Y_Y407A/T366A_K409F, L351Y_Y407A/T366V_K409F, K392D/D399K, K392D/E356K, K253E_D282K_K322D/D239K_E240K_K292D, K392D_K409D/D356K_D399K as described in WO2007/147901, WO 2011/143545, WO2013157954, WO2013096291 and US2018/0118849.

### Linkers

The isolated molecules or the multispecific antibodies of the disclosure may also comprise linkers connecting one or more antigen binding domains to the VH, the VL, the CH1 domain, the CL domain, the CH2 domain, the CH3 domain, the Fc region or fragments thereof, albumin, PEG, transferrin, or to one another. Various linkers may be used, including synthetic sequences or sequences from native immunoglobulin hinge regions or fragments thereof, or modified hinge regions. Hinge regions may be derived from human or any other species, such as mouse, rat, rabbit, camel, llama, shark, goat or dog. Hinge regions may be of different isotype than the HC or Fc region that is used in the particular molecule of the disclosure. The hinge regions or fragments thereof may be modified by one or more substitution, such as substitutions that increase or decrease the number of cysteine residues in the hinge. Modified hinge regions are those disclosed for example in U.S. Pat. No. 5,677,425, WO9915549, WO2005003170, WO2005003169, WO2005003170, WO9825971 and WO2005003171. Exemplary hinge regions or fragments thereof or modified hinge regions are shown in Table 1.

TABLE 1

| | | SEQ |
|---|---|---|
| Hinge region name | Amino acid sequence | ID |
| H1 Human lgA1 | VPSTPPTPSPSTPPTPSPS | 2183 |
| H2 Human lgA2 | VPPPPP | 2184 |
| H3 Human IgD | ESPKAQASSVPTAQPQAEG | 2185 |
| | SLAKATTAPATTRNTGRGG | |
| | EEKKKEKEKEEQEERETKT | |
| | P | |
| H4 Human lgG1 | EPKSCDKTHTCPPCP | 2186 |
| H5 Human lgG2 | ERKCCVECPPCP | 2187 |
| H6 Human lgG3 | ELKTPLGDTTHTCPRCPEP | 2188 |
| | KSCDTPPPCPRCPEPKSCD | |
| | TPPPCPRCPEPKSCDTPPP | |
| | CPRCP | |
| H7 Human lgG4 | ESKYGPPCPSCP | 2189 |
| H8 Human lgG4(P) | ESKYGPPCPPCP | 2190 |
| H9 Engineered hinge v1 | CPPC | 2191 |
| H10 Engineered hinge v2 | CPSC | 2192 |
| H11 Engineered hinge v3 | CPRC | 2193 |
| H12 Engineered hinge v4 | SPPC | 2194 |
| H13 Engineered hinge v5 | CPPS | 2195 |
| H14 Engineered hinge v6 | SPPS | 2196 |
| H15 Engineered hinge v7 | DKTHTCAA | 2197 |
| H16 Engineered hinge v8 | DKTHTCPPCPA | 2198 |
| H17 Engineered hinge v9 | DKTHTCPPCPATCPPCPA | 2199 |
| H18 Engineered hinge | DKTHTCPPCPATCPPCPAT | 2200 |
| | CPPCPA | |
| H19 Engineered hinge | DKTHTCPPCPAGKPTLYNS | 2201 |
| | LVMSDTAGTCY | |
| H20 Engineered hinge | DKTHTCPPCPAGKPTHVNV | 2202 |
| | SVVMAEVDGTCY | |
| H21 Engineered hinge | DKTHTCCVECPPCPA | 2203 |
| H22 Engineered hinge | DKTHTCPRCPEPKSCDTPP | 2204 |
| | PCPRCPA | |
| H23 Engineered hinge | DKTHTCPSCPA | 2205 |

Synthetic linkers that may be used to connect the antigen binding domains to one another or the VH, the VL, the CH1 domain, the CL domain, the CH2 domain, the CH3 domain or the Fc region or fragments thereof include flexible and/or charged peptide linkers of varying length, such as linkers between from about 2 to about 60 amino acids. Synthetic linkers that may be used include those disclosed by Chen et al., 2013, Adv Drug Deliv Rev. 65(10): 1357-1369 and Klein et al., 2014, Protein Engineering, Design & Selection 27(10): 325-330. Exemplary suitable synthetic linkers are shown in Table 2.

TABLE 2

| Linker | | |
|---|---|---|
| name | Linker amino acid sequence | SEQ |
| L1 | ADAAP | 2206 |
| L2 | ADAAPTVSIFP | 2207 |
| L3 | ADAAPTVSIFPP | 2208 |
| L4 | AKTTAP | 2209 |
| L5 | AKTTAPSVYPLAP | 2210 |
| L6 | AKTTPKLEEGEFSEARV | 2211 |
| L7 | AKTTPKLGG | 2212 |
| L8 | AKTTPP | 2213 |
| L9 | AKTTPPSVTPLAP | 2214 |
| L10 | ASTKGP | 2215 |
| L11 | ASTKGPSVFPLAP | 2216 |
| L12 | ASTKGPSVFPLAPASTKGPSVFPLAP | 2217 |
| L13 | EGKSSGSGSESKST | 2218 |
| L14 | GEGESGEGESGEGES | 2219 |
| L15 | GEGESGEGESGEGESGEGES | 2220 |
| L16 | GEGGSGEGGSGEGGS | 2221 |
| L17 | GENKVEYAPALMALS | 2222 |
| L18 | GGEGSGGEGSGGEGS | 2223 |
| L19 | GGGESGGEGSGEGGS | 2224 |
| L20 | GGGESGGGESGGGES | 2225 |
| L21 | GGGGSGGGGS | 2226 |
| L22 | GGGGSGGGGSGGGGS | 2227 |
| L23 | GGGGSGGGGSGGGGSGGGGS | 2228 |
| L24 | GGGKSGGGKSGGGKS | 2229 |
| L25 | GGGKSGGKGSGKGGS | 2230 |
| L26 | GGKGSGGKGSGGKGS | 2231 |
| L27 | GGSGG | 2232 |
| L28 | GGSGGGGSGGGGS | 2233 |
| L29 | GHEAAAVMQVQYPAS | 2234 |
| L30 | GKGGSGKGGSGKGGS | 2235 |
| L31 | GKGKSGKGKSGKGKS | 2236 |
| L32 | GKGKSGKGKSGKGKSGKGKS | 2237 |
| L33 | GKPGSGKPGSGKPGS | 2238 |
| L34 | GKPGSGKPGSGKPGSGKPGS | 2239 |
| L35 | GPAKELTPLKEAKVS | 2240 |
| L36 | GSAGSAAGSGEF | 2241 |
| L37 | IRPRAIGGSKPRVA | 2242 |
| L38 | KESGSVSSEQLAQFRSLD | 2243 |
| L39 | KTTPKLEEGEFSEAR | 2244 |
| L40 | QPKAAP | 2245 |
| L41 | QPKAAPSVTLFPP | 2246 |
| L42 | RADAAAAGGPGS | 2247 |
| L43 | RADAAP | 2248 |
| L44 | RADAAPTVS | 2249 |
| L45 | SAKTTP | 2250 |
| L46 | SAKTTPKLEEGEFSEARV | 2251 |
| L47 | SAKTTPKLGG | 2252 |
| L48 | STAGDTHLGGEDFD | 2253 |
| L49 | TVAAP | 2254 |
| L50 | TVAAPSVFIFPP | 2255 |
| L51 | TVAAPSVFIFPPTVAAPSVFIFPP | 2256 |
| L52 | RADAAAA(G4S)4 | 2257 |
| L53 | GGSEGKSSGSGSESKSTGGS | 2258 |
| L54 | GGGSGGGS | 2259 |
| L55 | GGGSGGGSGGGS | 2260 |
| L56 | GGGSGGGSGGGSGGGS | 2261 |
| L57 | GGGSGGGSGGGSGGGSGGGS | 2262 |
| L58 | GGGGSGGGGSGGGGS | 2263 |
| L59 | GGGGSGGGGSGGGGSGGGGS | 2264 |
| L60 | GGGGSGGGGSGGGGSGGGGSGGGGS | 2265 |
| L61 | GSTSGSGKPGSGEGSTKG | 2266 |
| L62 | IRPRAIGGSKPRVA | 2267 |
| L63 | GKGGSGKGGSGKGGS | 2268 |
| L64 | GGKGSGGKGSGGKGS | 2269 |
| L65 | GGGKSGGGKSGGGKS | 2270 |
| L66 | GKGKSGKGKSGKGKS | 2271 |
| L67 | GGGKSGGKGSGKGGS | 2272 |
| L68 | GKPGSGKPGSGKPGS | 2273 |
| L69 | GKPGSGKPGSGKPGSGKPGS | 2274 |
| L70 | GKGKSGKGKSGKGKSGKGKS | 2275 |
| L71 | STAGDTHLGGEDFD | 2276 |
| L72 | GEGGSGEGGSGEGGS | 2277 |
| L73 | GGEGSGGEGSGGEGS | 2278 |
| L74 | GEGESGEGESGEGES | 2279 |
| L75 | GGGESGGEGSGEGGS | 2280 |
| L76 | GEGESGEGESGEGESGEGES | 2281 |
| L77 | GSTSGSGKPGSGEGSTKG | 2282 |
| L78 | PRGASKSGSASQTGSAPGS | 2283 |
| L79 | GTAAAGAGAAGGAAAGAAG | 2284 |
| L80 | GTSGSSGSGSGGSGSGGGG | 2285 |
| L81 | GKPGSGKPGSGKPGSGKPGS | 2286 |
| L82 | GSGS | 2287 |
| L83 | APAPAPAPAP | 2288 |
| L84 | APAPAPAPAPAPAPAPAPAP | 2289 |
| L85 | AEAAAKEAAAKEAAAAKEAAAAKEAAAAKAAA | 2290 |

### Isotypes, Allotypes and Fc Engineering

The isolated molecules or the isolated multispecific antibodies of the disclosure may be of any isotype or allotype in instances when a portion of a full heavy chain is present in the molecules or in the multispecific antibodies.

It is expected that allotype has no influence on properties of isolated molecules or the isolated multispecific antibodies of the disclosure, such as specific binding to an antigen or Fc-mediated effector functions or half-life. Allotype is related to amino acid sequence variations at specific locations in the constant region sequences of a heavy chain of an immunoglobulin. Table 3 shows select IgG1, IgG2 and IgG4 allotypes.

TABLE 3

| | Amino acid residue at position of diversity | | | | | | | |
|---|---|---|---|---|---|---|---|---|
| | (residue numbering: EU Index) | | | | | | | |
| | IgG2 | | IgG4 | | IgG1 | | | |
| Allotype | 189 | 282 | 309 | 422 | 214 | 356 | 358 | 431 |
| G2m(n) | T | M | | | | | | |
| G2m(n−) | P | V | | | | | | |
| G2m(n)/(n− | T | V | | | | | | |
| nG4m(a) | | | L | R | | | | |
| G1m(17) | | | | | K | E | M | A |
| G1m(17,1) | | | | | K | D | L | A |

When present, C-terminal lysine may be removed from the isolated molecules or the isolated multispecific antibodies of the disclosure by endogenous circulating carboxypeptidases in the blood stream (Cai et al., (2011) Biotechnol Bioeng 108:404-412). During manufacturing, CTL removal may be controlled to less than the maximum level by control of concentration of extracellular Zn²⁺, EDTA or EDTA-Fe³⁺ as described in U.S. Patent Publ. No. US20140273092. C-terminal lysine content of proteins may be measured using known methods. In some embodiments, the isolated molecule or the isolated multispecific antibody of the disclosure has a C-terminal lysine content from about 10% to about 90%. In some embodiments, the C-terminal lysine content is from about 20% to about 80%. In some embodiments, the C-terminal lysine content is from about 40% to about 70%. In some embodiments, the C-terminal lysine content is from about 55% to about 70%. In some embodiments, the C-terminal lysine content is about 60%.

The Fc region (Fc), when present in the isolated molecules or the isolated multispecific antibodies of the disclosure, may comprise at least one substitution in the Fc region which modulates Fc-mediated effector functions CDC, ACC, ADCP by modulating binding to activating or inhibitory FcγR or FcRn, or which modulates protein A binding to facilitate purification. Fc positions that may be substituted to reduce binding of the isolated molecule or the isolated multispecific antibody of the disclosure to the activating FcγR and subsequently to reduce effector function include positions 214, 233, 234, 235, 236, 237, 238, 265, 267, 268, 270, 295, 297, 309, 327, 328, 329, 330, 331 and 365. Exemplary substitutions that may be made singularly or in combination are substitutions K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S in IgG1, IgG2, IgG3 or IgG4.

Exemplary combination substitutions that may be made to reduce ADCC are mutations L234A/L235A on IgG1, L234A/L235A/D265S on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4. Hybrid IgG2/4 Fc domains may also be used, such as Fc with residues 117-260 from IgG2 and residues 261-447 from IgG4.

Exemplary substitution that may be used to reduce CDC is a K322A mutation.

Fc positions that may be substituted to enhance binding of the isolated molecule or the isolated multispecific antibody of the disclosure to the activating FcγR and/or enhance Fc effector functions include positions 236, 239, 243, 256, 290, 292, 298, 300, 305, 312, 326, 330, 332, 333, 334, 345, 360, 339, 378, 396 or 430 (residue numbering according to the EU index). Exemplary mutations that may be made singularly or in combination are G236A, S239D, F243L, T256A, K290A, R292P, S298A, Y300L, V305L, K326A, A330K, 1332E, E333A, K334A, A339T and P396L. Exemplary combination substitutions that may be made to enhance ADCC or ADCP are S239D/I332E, S298A/E333A/K334A, F243L/R292P/Y300L, F243L/R292P/Y300L/P396L, F243L/R292P/Y300L/V3051/P396L or G236A/S239D/I332E. Fc positions that may be substituted to enhance CDC include positions 267, 268, 324, 326, 333, 345 and 430. Exemplary substitutions that may be made singularly or in combination are S267E, F1268F, S324T, K326A, K326W, E333A, E345K, E345Q, E345R, E345Y, E430S, E430F and E430T. Exemplary combination substitutions that may be made to enhance CDC are K326A/E333A, K326W/E333A, H268F/S324T, S267E/H268F, S267E/S324T and S267E/H268F/S324T.

In some embodiments, the FcγR is FcγRI, FcγRIIA, FcγRIIB or FcγRIII, or any combination thereof.

Fc positions that may be substituted to modulate half-life (e.g., binding to FcRn) include positions 250, 252, 253, 254, 256, 257, 307, 376, 380, 428, 434 and 435. Exemplary substitutions that may be made singularly or in combination are mutations T250Q, M252Y, I253A, S254T, T256E, P2571, T307A, D376V, E380A, M428L, H433K, N434S, N434A, N434H, N434F, H435A and H435R. Exemplary singular or combination substitutions that may be made to increase the half-life are substitutions M428L/N434S, M252Y/S254T/T256E, T250Q/M428L, N434A and T307A/E380A/N434A. M252Y/S254T/T256E is particularly useful. Exemplary singular or combination substitutions that may be made to reduce the half-life are mutations H435A, P2571/N434H, D376V/N434H, M252Y/S254T/T256E/H433K/N434F, T308P/N434A and H435R.

The specific substitutions described herein are substitutions when compared to the wild-type IgG1, wild-type IgG2 and wild-type IgG4 amino acid sequences of SEQ ID NOs: 2315, 2316 and 2317, respectively.

Exemplary substitutions that may be used in molecules that comprise two Fc regions are: L235A_L235A_D265S_T350V_L351Y_F405A_Y407V in the first Fc region and L235A_L235A_D265S_T350V_T366L_K392L_T394W in the second Fc region; or L235A_L235A_D265S_T350V_T366L_K392L_T394W in the first Fc region and L235A_L235A_D265S_T350V_L351Y_F405A_Y407V in the second Fc region.

(wild-type IgG1)
SEQ ID NO: 2315
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
KVEPKSCDKTHTCPPCPAPELLGGPSVFLEPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFF
LYSKLTVDKSRWQQGNVFSCSVMHEALHNHYTQKSLSLSPGK
(wild-type IgG2)
SEQ ID NO: 2316
ASTKGPSVFPLAPCSRSTSESTAALGCLVKDYFPEPVTVSWNSGALTS
GVHTFPAVLQSSGLYSLSSVVTVPSSNFGTQTYTCNVDHKPSNTKVDK
TVERKCCVECPPCPAPPVAGPSVFLFPPKPKDTLMISRTPEVTCVVVD
VSHEDPEVQFNWYVDGVEVHNAKTKPREEQFNSTERVVSVLTVVHQDW
LNGKEYKCKVSNKGLPAPIEKTISKTKGQPREPQVYTLPPSREEMTKN
QVSLTCLVKGFYPSDISVEWESNGQPENNYKTTPPMLDSDGSFFLYSK
LTVDKSRWQQGNVFSCSVMHEALHNHYTQKSLSLSPGK
(wild-type IgG4)
SEQ ID NO: 2317
ASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDK
RVESKYGPPCPSCPAPEFLGGPSVFLEPPKPKDTLMISRTPEVTCVVV
DVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYS
RLTVDKSRWQEGNVFSCSVMHEALHNHYTQKSLSLSLGK

Binding of the molecule or the multispecific antibody of the disclosure to FcγR or FcRn may be assessed on cells engineered to express each receptor using flow cytometry. In an exemplary binding assay, 2×10⁵ cells per well are seeded in 96-well plate and blocked in BSA Stain Buffer (BD Biosciences, San Jose, USA) for 30 min at 4° C. Cells are incubated with a test molecule on ice for 1.5 hour at 4° C. After being washed twice with BSA stain buffer, the cells are incubated with R-PE labeled anti-human IgG secondary antibody (Jackson Immunoresearch Laboratories) for 45 min at 4° C. The cells are washed twice in stain buffer and then resuspended in 150 μL of Stain Buffer containing 1:200 diluted DRAQ7 live/dead stain (Cell Signaling Technology, Danvers, USA). PE and DRAQ7 signals of the stained cells are detected by Miltenyi MACSQuant flow cytometer (Miltenyi Biotec, Auburn, USA) using B2 and B4 channel respectively. Live cells are gated on DRAQ7 exclusion and the geometric mean fluorescence signals are determined for at least 10,000 live events collected. FlowJo software (Tree Star) is used for analysis. Data is plotted as the logarithm of antibody concentration versus mean fluorescence signals. Nonlinear regression analysis is performed.

“Antibody-dependent cellular cytotoxicity”, “antibody-dependent cell-mediated cytotoxicity” or (ADCC) is a mechanism for inducing cell death that depends upon the interaction of antibody-coated target cells with effector cells possessing lytic activity, such as natural killer cells (NK), monocytes, macrophages and neutrophils via Fc gamma receptors (FcγR) expressed on effector cells. For example, NK cells express FcγRIIIa, whereas monocytes express FcγRI, FcγRII and FcγRIIIa. ADCC activity of the antibodies may be assessed using an in vitro assay using cells expressing the antigen the molecule or the multispecific antibody of the disclosure specifically binds to and NK cells as effector cells. Cytolysis may be detected by the release of label (e.g., radioactive substrates, fluorescent dyes or natural intracellular proteins) from the lysed cells. In an exemplary assay, target cells are used with a ratio of 1 target cell to 4 effector cells. Target cells are pre-labeled with BATDA and combined with effector cells and the test antibody. The samples are incubated for 2 hours and cell lysis measured by measuring released BATDA into the supernatant. Data is normalized to maximal cytotoxicity with 0.67% Triton X-100 (Sigma Aldrich) and minimal control determined by spontaneous release of BATDA from target cells in the absence of any antibody.

“Antibody-dependent cellular phagocytosis” (ADCP) refers to a mechanism of elimination of antibody-coated target cells by internalization by phagocytic cells, such as macrophages or dendritic cells. ADCP may be evaluated by using monocyte-derived macrophages as effector cells and cells expressing the antigen the molecule or the multispecific antibody of the disclosure specifically binds to as target cells also engineered to express GFP or another labeled molecule. In an exemplary assay, effector:target cell ratio may be for example 4:1. Effector cells may be incubated with target cells for 4 hours with or without the antibody of the invention. After incubation, cells may be detached using accutase. Macrophages may be identified with anti-CD11b and anti-CD14 antibodies coupled to a fluorescent label, and percent phagocytosis may be determined based on % GFP fluorescence in the CD11⁺CD14⁺ macrophages using standard methods.

“Complement-dependent cytotoxicity”, (CDC), refers to a mechanism for inducing cell death in which the Fc effector domain of a target-bound antibody binds and activates complement component C1q which in turn activates the complement cascade leading to target cell death. Activation of complement may also result in deposition of complement components on the target cell surface that facilitate CDC by binding complement receptors (e.g., CR3) on leukocytes. CDC of cells may be measured for example by plating cells expressing the antigen the molecule or the multispecific antibody of the disclosure specifically binds to at 1×10⁵ cells/well (50 μL/well) in RPMI-B (RPMI supplemented with 1% BSA), adding 50 μL of test molecule to the wells at final concentration between 0-100 μg/mL, incubating the reaction for 15 min at room temperature, adding 11 μL of pooled human serum to the wells, and incubation the reaction for 45 min at 37° C. Percentage (%) lysed cells may be detected as % propidium iodide stained cells in FACS assay using standard methods.

The Fc engineered molecules or the multispecific antibodies of the disclosure may be assessed for their functionality using several known assays and those described herein. Soluble forms of the receptors, such as the FcγRI, FcγRII, FcγRIII or FcRn receptors may be used, or alternatively cell-based assays may be used.

Protein A binding may be modulated using substitutions 435R and/or 436F as described in U.S. Pat. No. 9,982,013 or Q311R, Q311K, T307P/L309Q, T307P/V309Q, T307P/L309Q/Q311R or T307P/V309Q/Q311R as described in Int. Pat. Publ. No. WO2018/224951. Typically substations modulating protein A binding are engineered in asymmetric fashion to facilitate purification of the desired end product from intermediate or parental products.

### Half-Life Extension

Various additional approaches in addition to incorporating Fc region and introducing FcRn modulating substitutions into the Fc may be taken to modulate half-life of the molecules of the disclosures. The molecules of the disclosure may be pegylated, conjugated to albumin, albumin binding proteins transferring and fragments or analogues thereof, or XTEN polypeptide sequences (Int Pat. Publ. No. WO2010/091122) using known methods.

Additional half-life extending moieties that may be conjugated to molecules of the disclosure include polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the molecules of the disclosure and may be generated by standard cloning and expression techniques. Alternatively, well known chemical coupling methods may be used to attach the moieties to recombinantly produced antigen binding domains that bind hK2 of the disclosure.

A pegyl moiety may for example be conjugated to the antigen binding domain by incorporating a cysteine residue to the C-terminus of the antigen binding domain, or engineering cysteines into residue positions that face away from the antigen binding site and attaching a pegyl group to the cysteine using well known methods.

### Glycoengineering

The isolated molecules or the isolated multispecific antibodies of the disclosure may be glycoengineered for the purpose of for example to facilitate manufacturing or to provide additional functionality. This can be accomplished for example by deleting or introducing N-glycosylation and/or O-glycosylation sites. Fc region containing molecules or the isolated multispecific antibodies may be converted to aglycosyl variants by N297A or N297Q substitution. Aglycosyl Fc variants may provide improved manufacturability in terms of more homogenous batches and also demonstrated reduced FcγR binding and hence reduced Fc-mediated effector functions.

Further, the isolated molecules or the isolated multispecific antibodies of the disclosure may also be expressed utilizing conditions that result in molecules having reduced amount of fucosyl residues or increased bisecting GlcNac structures. Such altered glycosylation patterns have been demonstrated to potentiate ADCC. These carbohydrate modifications may be accomplished by, for example, expressing the isolated molecules or the isolated multispecific antibodies of the disclosure in a cell with altered glycosylation machinery. Cells with altered glycosylation machinery have been described in the art and can be used as host cells in which to express the molecules of the disclosure to thereby produce molecules with altered glycosylation. For example, EP 1,176,195 describes a cell line with a functionally disrupted FUT8 gene, which encodes a fucosyl transferase, such that molecules expressed in such a cell line exhibit hypofucosylation. PCT Publication WO 03/03583 describes a variant CHO cell line, Lec13 cells, with reduced ability to attach fucose to Asn(297)-linked carbohydrates, also resulting in hypofucosylation of molecules expressed in that host cell (see also Shields et ai, 2002, J. Biol. Chem. 277:26733-26740). PCT Publication WO 99/54342 by Umana et al. describes cell lines engineered to express glycoprotein modifying glycosyl transferases (e.g., beta(1,4)-N acetylglucosaminyltransferase III (GnTIII)) such that molecules expressed in the engineered cell lines exhibit increased bisecting GlcNac structures which results in increased ADCC activity of the molecules (see also Umana et ai, Nat. Biotech. 17:176-180, 1999). Additionally, relatively high defucosylated molecules bearing the biantennary complex-type of Fc oligosaccharides may be generated by controlling culture osmolality (Konno et al., Cytotechnology 64(:249-65, 2012), application of a variant CHO line EB66 as the host cell line (Olivier et al., MAbs; 2(4): 405-415, 2010; PMID:20562582), application of a rat hybridoma cell line YB2/0 as the host cell line (Shinkawa et al., J Biol Chem 278:3466-3473, 2003), introduction of small interfering RNA specifically against the a 1,6-fucosyltrasferase (FUT8) gene (Mori et al., Biotechnol Bioeng 88:901-908, 2004), or co-expression of β-1,4-N-acetylglucosaminyltransferase III and Golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

### Co-Engagement of the TCR Complex and CD8

The isolated molecules or the multispecific antibodies of the disclosure are generated in a manner that results in CD8⁺ CTL activation only upon co-engagement of the TCR complex and CD8. Co-engagement and subsequent CD8⁺ CTL cell activation is controlled by choosing sufficiently low affinity CD8 and TCR complex antigen binding domains to be incorporated into the molecules or the multispecific antibodies. Using the low affinity binding domains, activation of CD8⁺ CTLs does not occur in molecules in which only either the low affinity CD8 binding domain or the low affinity TCR complex binding domain is present. The concept was successfully demonstrated herein as shown in Example 2. Molecules incorporating a low affinity CD3 binding domain without CD8 binding domains were unable to mediate tumor cell death or T cell activation, however incorporation of a CD8 binding domain into these molecules resulted in robust tumor cell death and T cell activation. On the contrary, molecules incorporating high affinity CD3 binding domains were able to mediate tumor cell killing in the absence of CD8 biding domains in the molecules.

The affinities of the antigen binding domains that specifically bind CD8 and the antigen binding domains that specifically bind the TCR complex that can be incorporated into the molecules or the multispecific antibodies of the disclosure may be in the range of about 50 nM or higher for an antigen binding domain that binds the TCR complex and about 0.5 nM or higher for an antigen binding domain that binds CD8. However, higher affinity antigen binding domains may also be used as long as they do not alone activate T cells.

Affinity of the antigen binding domains that bind CD8 or TCR complex or molecules comprising the antigen binding domains that specifically bind CD8 or TCR complex may be measured using known methods. The binding may be measured using Biacore 8K SPR. In an exemplary method, Biacore 8K SPR assay format is to capture the test molecule (e.g., the antigen binding domain or the molecule comprising the antigen binding domain) using a high density anti-human Fc surface, then inject antigen concentration titration using a single cycle kinetics method. Goat anti-human Fc IgG (Jackson Immunoresearch, Cat #109-005-098) is directly immobilized via amine coupling at 30 μg/mL in 10 mM acetate buffer, pH 4.5 on flow cells 1 and 2, on CMS Sensor Chip (GE) with a flow rate of 30 μL/min in HBSP (GE) buffer. The test molecules are captured on the anti-human Fc IgG surface at 0.5 μg/ml (˜200-300 RU) on flow cell 2. The running buffer is then changed to HBSP+100 ug/ml BSA. Antigen at 30 nM concentration in 3-fold dilution series is injected from low to high concentration using single cycle kinetics method. The off-rate is monitored 30 minutes after the last or highest concentration injection and then the surface is regenerated using 0.8% phosphoric acid (Bio-Rad). A buffer blank run, capturing the same test molecule and using the same conditions of sample run is also completed. The raw data is processed by subtracting two sets of reference data from the response data: 1) reference flow cell 1 subtracted from sample flow cell 2 and 2) buffer blank run from experimental run. The processed data at all concentrations for each test molecule is globally fit to a 1:1 simple Langmuir binding model to extract estimates of the kinetic (kon, koff) and affinity (KD) constants.

The affinity of the third antigen binding domain that specifically binds an antigen expressed by an undesired cell may be determined using methods described herein. The affinity of the third antigen binding domain may range substantially and typically may be about 1×10⁻⁸ or less.

The effect of the molecule or the multispecific antibody on T cell activation may be assessed for example evaluating T cell proliferation in an assay in which human Pan T cell are isolated from healthy human donor PBMCs using for example EasySep™ Human T Cell Enrichment Kit, culturing the isolated T cells in a 1:1 Effector:Target ratio (10,000 T cells:10,000 target cells) at varying test molecule concentrations starting from 500 ng/ml, with 3-fold serial dilution. Suitable target cells are for example H929 cells. T cells ware labeled with CellTrace™ Violet (CTV) Cell Proliferation dye Kit (ThermoFisher) prior to co-culture. After 72 hrs, samples are harvested, labeled with anti-CD3 and anti-CD8 antibody and analyzed for CTV dye dilution. Cells are gated for FSC/SSC, live cells and CD3+CD8+ or CD3+CD8-cells. Alternatively, CD25 may be used as surrogate for T cell activation.

Conjugates with Cytotoxic Agents, Drugs, Detectable Labels, and the Like

The isolated molecules or the multispecific molecules of the disclosure may be conjugated to a cytotoxic agent, therapeutic agent, detectable labels and the like. These molecules are referred herein to immunoconjugates. The immunoconjugates comprising the isolated molecules or the multispecific molecules of the disclosure may be used to detect, deliver payload or kill cells the undesired cells the molecules or the multispecific molecules of the disclosure bind to. Alternatively, the immunoconjugates comprising the isolated molecules or the multispecific molecules of the disclosure may be used to detect, deliver payload or kill the CD8⁺ CTLs in instances when the molecules or the multispecific molecules of the disclosure do not comprise the thirds antigen binding domain that binds an antigen expressed by an undesired cell, e.g., bispecific CD3×CD8 molecules.

In some embodiments, the immunoconjugate comprises a detectable label.

In some embodiments, the immunoconjugate comprises a cytotoxic agent.

In some embodiments, the immunoconjugate comprises a therapeutic.

A detectable label includes compositions that can be visualized via spectroscopic, photochemical, biochemical, immunochemical, or chemical means. Detectable labels may also include cytotoxic agents, cytotoxic agents may include detectable labels.

Exemplary detectable labels include radioactive isotopes, magnetic beads, metallic beads, colloidal particles, fluorescent dyes, electron-dense reagents, enzymes (for example, as commonly used in an ELISA), biotin, digoxigenin, haptens, luminescent molecules, chemiluminescent molecules, fluorochromes, fluorophores, fluorescent quenching agents, colored molecules, radioactive isotopes, scintillates, avidin, streptavidin, protein A, protein G, antibodies or fragments thereof, polyhistidine, Ni²⁺, Flag tags, myc tags, heavy metals, enzymes, alkaline phosphatase, peroxidase, luciferase, electron donors/acceptors, acridinium esters, and colorimetric substrates.

A detectable label may emit a signal spontaneously, such as when the detectable label is a radioactive isotope. In other cases, the detectable label emits a signal as a result of being stimulated by an external field.

Exemplary radioactive isotopes may be γ-emitting, Auger-emitting, β-emitting, an alpha-emitting or positron-emitting radioactive isotope. Exemplary radioactive isotopes include ³H, ¹¹C, ¹³C, ¹⁵N, ¹⁸F, ¹⁹F, ⁵⁵Co, ⁵⁷Co, ⁶⁰Co, ⁶¹Cu, ⁶²Cu, ⁶⁴Cu, ⁶⁷Cu, ⁶⁸Ga, ⁷²As, ⁷⁵Br, ⁸⁶Y, ⁸⁹Zr, ⁹⁰Sr, ⁹⁴mTc, ⁹⁹mTc, ¹¹⁵In, ¹²³I, ¹²⁴I, ¹²⁵I, ¹³¹I, ²¹¹At, ²¹²Bi, ²¹³Bi, ²²³Ra, ²²⁶Ra, ²²⁵Ac and ²²⁷Ac.

Exemplary metal atoms are metals with an atomic number greater than 20, such as calcium atoms, scandium atoms, titanium atoms, vanadium atoms, chromium atoms, manganese atoms, iron atoms, cobalt atoms, nickel atoms, copper atoms, zinc atoms, gallium atoms, germanium atoms, arsenic atoms, selenium atoms, bromine atoms, krypton atoms, rubidium atoms, strontium atoms, yttrium atoms, zirconium atoms, niobium atoms, molybdenum atoms, technetium atoms, ruthenium atoms, rhodium atoms, palladium atoms, silver atoms, cadmium atoms, indium atoms, tin atoms, antimony atoms, tellurium atoms, iodine atoms, xenon atoms, cesium atoms, barium atoms, lanthanum atoms, hafnium atoms, tantalum atoms, tungsten atoms, rhenium atoms, osmium atoms, iridium atoms, platinum atoms, gold atoms, mercury atoms, thallium atoms, lead atoms, bismuth atoms, francium atoms, radium atoms, actinium atoms, cerium atoms, praseodymium atoms, neodymium atoms, promethium atoms, samarium atoms, europium atoms, gadolinium atoms, terbium atoms, dysprosium atoms, holmium atoms, erbium atoms, thulium atoms, ytterbium atoms, lutetium atoms, thorium atoms, protactinium atoms, uranium atoms, neptunium atoms, plutonium atoms, americium atoms, curium atoms, berkelium atoms, californium atoms, einsteinium atoms, fermium atoms, mendelevium atoms, nobelium atoms, or lawrencium atoms.

In some embodiments, the metal atoms may be alkaline earth metals with an atomic number greater than twenty. In some embodiments, the metal atoms may be lanthanides. In some embodiments, the metal atoms may be actinides. In some embodiments, the metal atoms may be transition metals. In some embodiments, the metal atoms may be poor metals. In some embodiments, the metal atoms may be gold atoms, bismuth atoms, tantalum atoms, and gadolinium atoms. In some embodiments, the metal atoms may be metals with an atomic number of 53 (i.e., iodine) to 83 (i.e., bismuth).

In some embodiments, the metal atoms may be atoms suitable for magnetic resonance imaging.

The metal atoms may be metal ions in the form of +1, +2, or +3 oxidation states, such as Ba²⁺, Bi³⁺, Cs⁺, Ca²⁺, Cr²⁺, Cr³⁺, Cr⁶⁺, Co²⁺, Co³⁺, Cu⁺, Cu²⁺, Cu³⁺, Ga³⁺, Gd³⁺, Au⁺, Au³⁺, Fe²⁺, Fe³⁺, F³⁺, Pb²⁺, Mn²⁺, Mn³⁺, Mn⁴⁺, Mn⁷⁺, Hg²⁺, Ni²⁺, Ni³⁺, Ag⁺, Sr²⁺, Sn²⁺, Sn⁴⁺, and Zn²⁺. The metal atoms may comprise a metal oxide, such as iron oxide, manganese oxide, or gadolinium oxide.

Suitable dyes include any commercially available dyes such as, for example, 5(6)-carboxyfluorescein, IRDye 680RD maleimide or IRDye 800CW, ruthenium polypyridyl dyes, and the like.

Suitable fluorophores are fluorescein isothiocyanate (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), near infrared (NIR) (700-900 nm) fluorescent dyes, and carbocyanine and aminostyryl dyes.

The immunoconjugates comprising a detectable label may be used as an imaging agent.

In some embodiments, the cytotoxic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

In some embodiments, the cytotoxic agent is daunomycin, doxorubicin, methotrexate, vindesine, bacterial toxins such as diphtheria toxin, ricin, geldanamycin, maytansinoids or calicheamicin. The cytotoxic agent may elicit their cytotoxic and cytostatic effects by mechanisms including tubulin binding, DNA binding, or topoisomerase inhibition.

In some embodiments, the cytotoxic agent is an enzymatically active toxin such as diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some embodiments, the cytotoxic agent is a radionuclide, such as ²¹²Bi, ¹³¹I, ¹³¹In, ⁹⁰Y, and ¹⁸⁶Re.

In some embodiments, the cytotoxic agent is dolastatins or dolostatin peptidic analogs and derivatives, auristatin or monomethyl auristatin phenylalanine. Exemplary molecules are disclosed in U.S. Pat. Nos. 5,635,483 and 5,780,588. Dolastatins and auristatins have been shown to interfere with microtubule dynamics, GTP hydrolysis, and nuclear and cellular division (Woyke et al (2001) Antimicrob Agents and Chemother. 45(12):3580-3584) and have anticancer and antifungal activity. The dolastatin or auristatin drug moiety may be attached to the antibody of the invention through the N (amino) terminus or the C (carboxyl) terminus of the peptidic drug moiety (WO02/088172), or via any cysteine engineered into the antibody.

The immunoconjugates may be made using known methods.

In some embodiments, the detectable label is complexed with a chelating agent.

The detectable label, cytotoxic agent or therapeutic may be linked directly, or indirectly via a linker, to the polypeptides, the heterologous polypeptides or the proteinaceous molecules that bind the polypeptides or the heterologous polypeptides. Suitable linkers are known in the art and include, for example, prosthetic groups, non-phenolic linkers (derivatives of N-succimidyl-benzoates; dodecaborate), chelating moieties of both macrocyclics and acyclic chelators, such as derivatives of 1,4,7,10-tetraazacyclododecane-1,4,7,10,tetraacetic acid (DOTA), derivatives of diethylenetriaminepentaacetic avid (DTPA), derivatives of S-2-(4-Isothiocyanatobenzyl)-1,4,7-triazacyclononane-1,4,7-triacetic acid (NOTA) and derivatives of 1,4,8,11-tetraazacyclodocedan-1,4,8,11-tetraacetic acid (TETA), N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene) and other chelating moieties. Suitable peptide linkers are well known.

### Kits

The disclosure also provides a kit comprising one or more isolated molecules or isolated multispecific antibodies of the disclosure. The kit may be used for therapeutic uses or as diagnostic kits.

In some embodiments, the kit comprises the isolated molecule or the isolated multispecific antibody of the disclosure and reagents for detecting the isolated molecule or the isolated multispecific antibody. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the isolated molecule or the isolated multispecific antibody for administration; pharmaceutically acceptable carriers; and devices or other materials for administration to a subject.

### Pharmaceutical Compositions

The disclosure also provides a pharmaceutical composition comprising the isolated molecule or the isolated multispecific antibody of the disclosure and a pharmaceutically acceptable carrier. For therapeutic use, the isolated molecule or the isolated multispecific antibody of the disclosure may be prepared as pharmaceutical compositions containing an effective amount of the isolated molecule or the isolated multispecific antibody of the disclosure as an active ingredient in a pharmaceutically acceptable carrier. “Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the antibodies of the invention in such pharmaceutical formulation may vary, from less than about 0.5%, usually to at least about 1% to as much as 15 or 20% by weight and may be selected primarily based on required dose, fluid volumes, viscosities, etc., according to the mode of administration selected. Suitable vehicles and formulations, inclusive of other human proteins, e.g., human serum albumin, are described, for example, in e.g., Remington: The Science and Practice of Pharmacy, 21st Edition, Troy, D. B. ed., Lipincott Williams and Wilkins, Philadelphia, Pa. 2006, Part 5, Pharmaceutical Manufacturing pp 691-1092, See especially pp. 958-989.

### Methods and Uses

The isolated molecules and the multispecific antibodies of the disclosure have broad applicability in therapeutic or research setting, as therapeutics, diagnostics, research tools, imaging agents and capture agents. The isolated molecules and the multispecific antibodies of the disclosure provide an improvement to the state of art by providing selective activation or recruitment of CD8⁺ CTLs and are thereby expected to provide more safe and effective treatment with a broader therapeutic index. The isolated molecules and the multispecific antibodies of the disclosure can be used to treat any diseases in which depletion or reduction in a number of undesired cells is desired. The isolated molecules and the multispecific antibodies of the disclosure may have a potential to treat patients without large naïve repertoire, such as elderly patients or any patients whose immune system is compromised.

The disclosure provides a method of targeting CD8⁺ CTLs to an undesired cell in a subject, comprising administering to the subject an isolated molecule comprising a first antigen binding domain, a second antigen binding domain and a third antigen binding domain, wherein the first antigen binding domain specifically binds CD8, the second antigen binding domain specifically binds a TCR complex and the third antigen binding domain specifically binds an antigen expressed by an undesired cell, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of targeting CD8⁺ CTLs to an undesired cell in a subject, comprising administering to the subject an isolated molecule comprising a first polypeptide, a second polypeptide and a third polypeptide, wherein the first polypeptide comprises, from N- to C-terminus, a second antigen binding domain comprising a scFv that specifically binds a TCR complex, a VH that is capable of specifically binding CD8, a CH1 domain, a hinge, a CH2 domain and a CH3 domain; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of targeting CD8⁺ CTLs to an undesired cell in a subject, comprising administering to the subject an isolated molecule comprising a first polypeptide, a second polypeptide and a third polypeptide, wherein the first polypeptide comprises, from N- to C-terminus, a VH that is capable of specifically binding CD8, a CH1 domain, a hinge, a CH2 domain and a CH3 domain; the second polypeptide comprises, from N- to C-terminus, a VL that is capable of specifically binding CD8, a CL domain and a second antigen binding domain comprising a scFv that specifically binds a TCR complex; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of targeting CD8⁺ CTLs to an undesired cell in a subject, comprising administering to the subject an isolated molecule comprising a first polypeptide, a second polypeptide and a third polypeptide, wherein the first polypeptide comprises, from N- to C-terminus, a VH that is capable of specifically CD8, a CH1 domain, a hinge, a CH2 domain, a CH3 domain and a second antigen binding domain comprising a scFv that specifically binds a TCR complex; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of treating a cancer in a subject, comprising: administering to the subject an isolated molecule comprising a first antigen binding domain, a second antigen binding domain and a third antigen binding domain, wherein the first antigen binding domain specifically binds CD8, the second antigen binding domain specifically binds a TCR complex and the third antigen binding domain specifically binds an antigen expressed by an undesired cell, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of treating a cancer in a subject, comprising administering to the subject an isolated molecule comprising a first polypeptide, a second polypeptide and a third polypeptide, wherein the first polypeptide comprises, from N- to C-terminus, a second antigen binding domain comprising a scFv that specifically binds a TCR complex, a VH that is capable of specifically binding CD8, a CH1 domain, a hinge, a CH2 domain and a CH3 domain; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of treating a cancer in a subject, comprising administering to the subject an isolated molecule comprising a first polypeptide, a second polypeptide and a third polypeptide, wherein the first polypeptide comprises, from N- to C-terminus, a VH that is capable of specifically binding CD8, a CH1 domain, a hinge, a CH2 domain and a CH3 domain; the second polypeptide comprises, from N- to C-terminus, a VL that is capable of specifically binding CD8, a CL domain and a second antigen binding domain comprising a scFv that specifically binds a TCR complex; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of treating a cancer in a subject, comprising administering to the subject an isolated molecule comprising a first polypeptide, a second polypeptide and a third polypeptide, wherein the first polypeptide comprises, from N- to C-terminus, a VH that is capable of specifically CD8, a CH1 domain, a hinge, a CH2 domain, a CH3 domain and a second antigen binding domain comprising a scFv that specifically binds a TCR complex; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of enhancing a CD8⁺ CTL response against an undesired cell in a subject, comprising: administering to the subject an isolated molecule comprising a first antigen binding domain, a second antigen binding domain and a third antigen binding domain, wherein the first antigen binding domain specifically binds CD8, the second antigen binding domain specifically binds TCR complex and the third antigen binding domain specifically binds an antigen expressed by an undesired cell, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of enhancing a CD8⁺ CTL response against an undesired cell in a subject, comprising administering to the subject an isolated molecule comprising a first polypeptide, a second polypeptide and a third polypeptide, wherein the first polypeptide comprises, from N- to C-terminus, a second antigen binding domain comprising a scFv that specifically binds a TCR complex, a VH that is capable of specifically binding CD8, a CH1 domain, a hinge, a CH2 domain and a CH3 domain; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of enhancing a CD8⁺ CTL response against an undesired cell in a subject, comprising administering to the subject an isolated molecule comprising a first polypeptide, a second polypeptide and a third polypeptide, wherein the first polypeptide comprises, from N- to C-terminus, a VH that is capable of specifically binding CD8, a CH1 domain, a hinge, a CH2 domain and a CH3 domain; the second polypeptide comprises, from N- to C-terminus, a VL that is capable of specifically binding CD8, a CL domain and a second antigen binding domain comprising a scFv that specifically binds a TCR complex; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of enhancing a CD8⁺ CTL response against an undesired cell in a subject, comprising administering to the subject an isolated molecule comprising a first polypeptide, a second polypeptide and a third polypeptide, wherein the first polypeptide comprises, from N- to C-terminus, a VH that is capable of specifically CD8, a CH1 domain, a hinge, a CH2 domain, a CH3 domain and a second antigen binding domain comprising a scFv that specifically binds a TCR complex; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of enhancing a CD8⁺ CTL response against a cancer in a subject, comprising: administering to the subject an isolated molecule comprising a first antigen binding domain, a second antigen binding domain and a third antigen binding domain, wherein the first antigen binding domain specifically binds CD8, the second antigen binding domain specifically binds a TCR complex and the third antigen binding domain specifically binds an antigen expressed by an undesired cell, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of enhancing a CD8⁺ CTL response against a cancer in a subject, comprising administering to the subject an isolated molecule comprising a first polypeptide, a second polypeptide and a third polypeptide, wherein the first polypeptide comprises, from N- to C-terminus, a second antigen binding domain comprising a scFv that specifically binds a TCR complex, a VH that is capable of specifically binding CD8, a CH1 domain, a hinge, a CH2 domain and a CH3 domain; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of enhancing a CD8⁺ CTL response against a cancer in a subject, comprising administering to the subject an isolated molecule comprising a first polypeptide, a second polypeptide and a third polypeptide, wherein the first polypeptide comprises, from N- to C-terminus, a VH that is capable of specifically binding CD8, a CH1 domain, a hinge, a CH2 domain and a CH3 domain; the second polypeptide comprises, from N- to C-terminus, a VL that is capable of specifically binding CD8, a CL domain and a second antigen binding domain comprising a scFv that specifically binds a TCR complex; and third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of enhancing a CD8⁺ CTL response against a cancer in a subject, comprising administering to the subject an isolated molecule comprising a first polypeptide, a second polypeptide and a third polypeptide, wherein the first polypeptide comprises, from N- to C-terminus, a VH that is capable of specifically CD8, a CH1 domain, a hinge, a CH2 domain, a CH3 domain and a second antigen binding domain comprising a scFv that specifically binds a TCR complex; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of providing an improved T cell redirection therapy to a subject in need thereof, comprising: administering to the subject an isolated molecule comprising a first antigen binding domain, a second antigen binding domain and a third antigen binding domain, wherein the first antigen binding domain specifically binds CD8, the second antigen binding domain specifically binds a TCR complex and the third antigen binding domain specifically binds an antigen expressed by an undesired cell, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of providing an improved T cell redirection therapy to a subject in need thereof, comprising: administering to the subject an isolated molecule comprising a first polypeptide, a second polypeptide and a third polypeptide, wherein the first polypeptide comprises, from N- to C-terminus, a second antigen binding domain comprising a scFv that specifically binds a TCR complex, a VH that is capable of specifically binding CD8, a CH1 domain, a hinge, a CH2 domain and a CH3 domain; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of providing an improved T cell redirection therapy to a subject in need thereof, comprising: administering to the subject an isolated molecule comprising a first polypeptide, a second polypeptide and a third polypeptide, wherein the first polypeptide comprises, from N- to C-terminus, a VH that is capable of specifically binding CD8, a CH1 domain, a hinge, a CH2 domain and a CH3 domain; the second polypeptide comprises, from N- to C-terminus, a VL that is capable of specifically binding CD8, a CL domain and a second antigen binding domain comprising a scFv that specifically binds a TCR complex; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of providing an improved T cell redirection therapy to a subject in need thereof, comprising: administering to the subject an isolated molecule comprising a first polypeptide, a second polypeptide and a third polypeptide, wherein the first polypeptide comprises, from N- to C-terminus, a VH that is capable of specifically CD8, a CH1 domain, a hinge, a CH2 domain, a CH3 domain and a second antigen binding domain comprising a scFv that specifically binds a TCR complex; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of selectively activating or recruiting CD8⁺ CTLs towards an undesired cell, comprising: contacting a population of lymphocytes with an isolated molecule, comprising: a first antigen binding domain, a second antigen binding domain and a third antigen binding domain, wherein the first antigen binding domain specifically binds CD8, the second antigen binding domain specifically binds a TCR complex and the third antigen binding domain specifically binds an antigen expressed by an undesired cell, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of selectively activating or recruiting CD8⁺ CTLs towards an undesired cell, comprising: contacting a population of lymphocytes with an isolated molecule comprising a first polypeptide, a second polypeptide and a third polypeptide, wherein the first polypeptide comprises, from N- to C-terminus, a second antigen binding domain comprising a scFv that specifically binds a TCR complex, a VH that is capable of specifically binding CD8, a CH1 domain, a hinge, a CH2 domain and a CH3 domain; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of selectively activating or recruiting CD8⁺ CTLs towards an undesired cell, comprising: contacting a population of lymphocytes with an isolated molecule comprising a first polypeptide, a second polypeptide and a third polypeptide, wherein the first polypeptide comprises, from N- to C-terminus, a VH that is capable of specifically binding CD8, a CH1 domain, a hinge, a CH2 domain and a CH3 domain; the second polypeptide comprises, from N- to C-terminus, a VL that is capable of specifically binding CD8, a CL domain and a second antigen binding domain comprising a scFv that specifically binds a TCR complex; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

The disclosure also provides a method of selectively activating or recruiting CD8⁺ CTLs towards an undesired cell, comprising: contacting a population of lymphocytes with an isolated molecule comprising a first polypeptide, a second polypeptide and a third polypeptide, wherein the first polypeptide comprises, from N- to C-terminus, a VH that is capable of specifically CD8, a CH1 domain, a hinge, a CH2 domain, a CH3 domain and a second antigen binding domain comprising a scFv that specifically binds a TCR complex; the second polypeptide comprises, from N- to C-terminus, a VL that is capable of specifically binding CD8 and a CL domain; and the third polypeptide comprises, from N- to C-terminus, a third antigen binding domain comprising a scFv that specifically binds an antigen expressed by an undesired cell and a Fc or a fragment of the Fc, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In some embodiments, the selective activation or recruitment of CD8⁺ CTLs comprises in vitro selective activation or recruitment of CD8⁺ CTLs.

In some embodiments, the selective activation or recruitment of CD8⁺ CTLs comprises ex vivo selective activation or recruitment of CD8⁺ CTLs.

In some embodiments, the selective activation or recruitment of CD8⁺ CTLs comprises in vivo selective activation or recruitment of CD8⁺ CTLs.

The disclosure also provides a method of selectively activating or recruiting CD8⁺ CTLs towards an undesired cell in a subject, comprising: administering to the subject an isolated molecule comprising a first antigen binding domain, a second antigen binding domain and a third antigen binding domain, wherein the first antigen binding domain specifically binds CD8, the second antigen binding domain specifically binds a TCR complex and the third antigen binding domain specifically binds an antigen expressed by an undesired cell, wherein the isolated molecule selectively activates or recruits CD8⁺ CTLs upon co-engagement of the TCR complex and CD8 and is unable to activate or recruit CD8⁺ CTLs in the absence of co-engagement of the TCR complex and CD8.

In some embodiments, the first antigen binding domain specifically binds CD8 and the second antigen binding domain specifically binds the TCR complex with affinities that result in activation or recruitment of CD8⁺ CTLs only upon co-engagement of the TCR complex and CD8.

In some embodiments, the first antigen binding domain, the second antigen binding domain or the third antigen binding domain comprises a scFv, a Fab, a Fab′, a F(ab′)₂, a Fd, a Fv, a domain antibody (dAb), a VHH, a VH, a LV, a non-antibody scaffold, or fragments thereof.

In some embodiments, the first antigen binding domain comprises the Fab In some embodiments, the second antigen binding domain comprises the scFv. In some embodiments, the third antigen binding domain comprises the scFv.

In some embodiments, the isolated molecule comprises: a first polypeptide comprising, from N- to C-terminus, the second antigen binding domain comprising the scFv, a VH that is capable of specifically binding CD8, a CH1 domain, a hinge, a CH2 domain and a CH3 domain; a second polypeptide comprising, from N- to C-terminus, a VL that is capable of specifically binding CD8 and a CL domain; and a third polypeptide comprising, from N- to C-terminus, the third antigen binding domain comprising the scFv and a Fc or a fragment of the Fc.

In some embodiments, the isolated molecule comprises: a first polypeptide comprising, from N- to C-terminus, a VH that is capable of specifically binding CD8, a CH1 domain, a hinge, a CH2 domain and a CH3 domain; a second polypeptide comprising, from N- to C-terminus, a VL that is capable of specifically binding CD8, a CL domain and the second antigen binding domain comprising the scFv; and a third polypeptide comprising, from N- to C-terminus, the third antigen binding domain comprising the scFv and a Fc or a fragment of the Fc.

In some embodiments, the isolated molecule comprises: a first polypeptide comprising, from N- to C-terminus, a VH that is capable of specifically binding CD8, a CH1 domain, a hinge, a CH2 domain, a CH3 domain and the second antigen binding domain comprising the scFv; a second polypeptide comprising, from N- to C-terminus, a VL that is capable of specifically binding CD8 and a CL domain; and a third polypeptide comprising, from N- to C-terminus, the third antigen binding domain comprising the scFv and a Fc or a fragment of the Fc.

In some embodiments, the first antigen binding domain comprising the Fab, the second antigen binding domain comprising the scFv or the third antigen binding domain comprising the scFv is conjugated to the Fc or the fragment of the Fc, to the VH that is capable of specifically biding CD8, to the CL domain or to the CH3 domain via a linker.

In some embodiments, the linker comprises a polypeptide of SEQ ID NOs: 2183-2290.

In some embodiments, the fragment of the Fc comprises a CH2 domain and a CH3 domain.

In some embodiments, the CH3 domain comprises one or more substitutions when compared to a wild-type CH3 domain.

In some embodiments, the one or more substitutions comprise T350V, L351Y, F405A, Y407V, T366Y, T366W, F405W, T394W, T394S, Y407T, Y407A, T366S/L368A/Y407V, L351Y/F405A/Y407V, T366I/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V or T350V/T366L/K392L/T394W, wherein residue numbering is according to the EU index.

In some embodiments, the first antigen binding domain comprising the Fab, the second antigen binding domain comprising the scFv or the third antigen binding domain comprising the scFv is conjugated to the Fc or the fragment of the Fc, to the VH that is capable of specifically biding CD8, to the CL domain or to the CH3 domain via a linker.

In some embodiments, the linker comprises a polypeptide of SEQ ID NOs: 2183-2290.

In some embodiments, the first polypeptide comprises a CH3 domain comprising one or more substitutions when compared to a wild-type CH3 domain which promote heterodimerization of the first polypeptide with the third polypeptide; the third polypeptide comprises a CH3 domain comprising one or more substitutions when compared to the wild-type CH3 domain which promote heterodimerization of the third polypeptide with the first polypeptide; or the first polypeptide comprises the CH3 domain comprising one or more substitutions when compared to the wild-type CH3 which promote heterodimerization of the first polypeptide with the third polypeptide and the third polypeptide comprises the CH3 domain comprising one or more substitutions when compared to the wild-type CH3 which promote heterodimerization of the third polypeptide with the first polypeptide.

In some embodiments, the one or more substitutions comprise T350V, L351Y, F405A, Y407V, T366Y, T366W, F405W, T394W, T394S, Y407T, Y407A, T366S/L368A/Y407V, L351Y/F405A/Y407V, T366I/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V or T350V/T366L/K392L/T394W, wherein residue numbering is according to the EU index.

In some embodiments, the Fc, the CH2 domain or the CH3 domain is an IgG1, IgG2, IgG3 or IgG4 isotype.

In some embodiments, the second antigen binding domain specifically binds CD3, TCRα chain, TCRβ chain, TCRγ chain or TCRδ chain, or any combination thereof.

In some embodiments, the TCRβ chain comprises TCRVB17.

In some embodiments, CD3 comprises CD3ε, CD3γ, CD3δ or CD3ζ.

In some embodiments, the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296.

In some embodiments, the second antigen binding domain that specifically binds CD3 comprises the VH of SEQ ID NO: 2297 and the VL of SEQ ID NO: 2298.

In some embodiments, the first antigen binding domain comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

In some embodiments, the first antigen binding domain comprises the VH of SEQ ID NO: 2313 and the VL of SEQ ID NO: 2314.

In some embodiments, the undesired cell is a pathogenic cell.

In some embodiments, the undesired cell is a cancer cell, an infected cell, a virus infected cell, a bacterial infected cell, an immune cell, an inflamed cell, a damaged cells, a foreign cell, an apoptotic cell, a dysplastic cell, an immunogenic cell, a metaplastic cell or a mutant cell, or any combination thereof.

In some embodiments, the subject has a cancer, a viral infection, or an immune-mediated disease.

In some embodiments, the cancer is a hematological malignancy or a solid tumor.

In some embodiments, the hematological malignancy comprises acute lymphoblastic leukemia, acute myeloid leukemia, anaplastic large-cell lymphoma, Burkitt's lymphoma, chronic lymphocytic leukemia, chronic myeloid leukemia, diffuse large B-cell lymphoma, dendritic cell neoplasm, follicular lymphoma, hairy cell leukemia, Hodgkin's lymphoma, leukemia, B cell leukemia, T cell leukemia, light chain amyloidosis, lymphoma, B cell lymphoma, NK cell lymphoma, T cell lymphoma, mantle-cell lymphoma, marginal zone B-cell lymphoma, monoclonal gammopathy of undetermined significance, mucosa-associated lymphatic tissue lymphoma, multiple myeloma, myelodysplastic syndrome, non-Hodgkin's lymphoma, plasma cell leukemia, precursor B-cell lymphoblastic leukemia, smoldering multiple myeloma or Waldenstrom's macroglobulinemia, or any combination thereof. In some embodiments, hematological malignancy comprises B cell malignancies. In some embodiments, hematological malignancy comprises T cell malignancies. In some embodiments, hematological malignancy comprises NK cell malignancies.

Exemplary B-cell non-Hodgkin's lymphomas are a lymphomatoid granulomatosis, a primary effusion lymphoma, an intravascular large B-cell lymphoma, a mediastinal large B-cell lymphoma, heavy chain diseases (including γ, μ, and a disease), lymphomas induced by therapy with immunosuppressive agents, such as cyclosporine-induced lymphoma, and methotrexate-induced lymphoma.

In some embodiments, the solid tumor comprises adenocarcinoma, anal cancer, basal cell carcinoma, biliary tract cancer, bladder cancer, bone cancer, breast cancer, cancer associated with infection, cancer of the adrenal gland, cancer of the endocrine system, cancer of the head or neck, cancer of the parathyroid gland, cancer of the penis, cancer of the thyroid gland, cancer of the urethra, cervical cancer, carcinoma of the breast, carcinoma of the fallopian tubes, carcinoma of the liver, carcinoma of the lung, carcinoma of the prostate, carcinoma of the renal pelvis, carcinoma of the vagina, carcinoma of the vulva, choriocarcinoma, clear cell carcinoma, colon cancer, colon carcinoma, colorectal cancer, connective tissue cancer, cutaneous or intraocular malignant melanoma, environmentally induced cancer, gastric cancer, gastrointestinal cancer, glioma, glioblastoma, endometrial cancer, epithelial cancer, esophageal cancer, eye cancer, larynx cancer, liver cancer, hepatocellular carcinoma, hormone refractory prostate adenocarcinoma, Kaposi's sarcoma, kidney cancer, lung cancer gastro-esophageal cancer, melanoma, mesothelioma, Merkel cell cancer, neuroblastoma, non-small cell lung cancer (NSCLC), osteosarcoma, ovarian cancer, pancreatic cancer, prostate cancer, rectal cancer, renal cell carcinoma, retinoblastoma rhabdomyosarcoma, squamous cell cancer, soft tissue sarcoma, solid tumors of childhood, spinal axis tumor, stomach cancer, testicular cancer, thyroid cancer, uterine cancer, urothelial carcinoma or sarcomas, or any combination thereof.

In some embodiments, the cancer is a relapsed cancer. In some embodiments, the cancer is a refractor cancer. In some embodiments, the subject is treatment naïve.

In some embodiments, the viral infection is infection with adenovirus, arboviral encephalitis virus, coronavirus, coxsackie virus, cytomegalovirus (CMV), dengue virus, echovirus, Epstein Barr virus, flaviviruses, human immunodeficiency virus (HIV), hepatitis A virus, hepatitis B virus, hepatitis C virus, herpes virus, HTLV virus, influenza virus, JC virus, measles virus, molluscum virus, mumps virus, papillomavirus, parvovirus, poliovirus, rabies virus, respiratory syncytial virus, rhinovirus, rotavirus, rubella virus or vaccinia virus, bacteria, virus, fungi, protozoa, parasite or prion, or any combination thereof.

In some embodiments, the immune-mediated disease is an autoimmune disease or an inflammatory disease. In some embodiments, the autoimmune disease comprises systemic lupus erythematosus (SLE), ankylosing spondylitis, Chagas disease, chronic obstructive pulmonary disease, Crohn's Disease, dermatomyositis, diabetes mellitus type 1, endometriosis, Goodpasture's syndrome, Graves' disease, Guillain-Barre syndrome (GBS), Hashimoto's disease, hidradenitis suppurativa, Kawasaki disease, IgA nephropathy, idiopathic thrombocytopenic purpura, interstitial cystitis, mixed connective tissue disease, morphea, multiple sclerosis, myasthenia gravis, narcolepsy, neuromyotonia, pemphigus vulgaris, pernicious anaemia, psoriasis, psoriatic arthritis, polymyositis, primary biliary cirrhosis, relapsing polychondritis, rheumatoid arthritis (RA), sarcoidosis, schizophrenia, scleroderma, Sjogren's syndrome, temporal arteritis, ulcerative colitis, vasculitis, vitiligo, Wegener's granulomatosis, IgG4-related disease, anti-synthetase syndrome, and autoimmunity associated with immunodeficiency including chronic variable immunodeficiency, Wiskott-Aldrich syndrome, Good syndrome, IgA deficiency, Hyper IgM syndrome, and complement disorders. In some embodiments, the subject to has or likely to develop allograft rejection.

In some embodiments, subjects have an autoantibody-associated condition. In some embodiments, the an autoantibody-associated condition comprises seropositive RA, SLE, postmyocardial infarction syndrome, subacute bacterial endocarditis, anti-glomerular basement membrane nephritis, autoimmune hepatitis, primary biliary cirrhosis, alopecia areata, bullous pemphigoid, cicatricial pemphigoid, dermatitis herpetiformis, gestational pemphigoid, pemphigus vulgaris, systemic scleroderma, Addison's disease, autoimmune polyendocrine syndrome type 2, autoimmune pancreatitis, diabetes mellitus type 1, autoimmune thyroiditis, Graves' disease, Sjogren's syndrome, celiac disease, antiphospholipid syndrome, autoimmune thrombocytopenic purpura, cold agglutinin disease, pernicious anemia, thrombocytopenia, adult onset Still's disease, CREST syndrome, drug-induced lupus, enthesitis-related arthritis, juvenile arthritis, mixed connective tissue disease, palindromic rheumatism, Parry Romberg syndrome, rheumatic fever, undifferentiated connective tissue disease, dermatomysitis, myasthenia gravis, neuromyotonia, paraneoplastic cerebellar degeneration, polymyositis, Bickerstaff s encephalitis, chronic inflammatory demyelinating polyneuropathy, Guillain-Barre syndrome, Hashimoto's encephalopathy, Lambert-Eaton myasthenic syndrome, multiple sclerosis, progressive inflammatory neuropathy, Stiff person syndrome, autoimmune uveitis, neuromyelitis optica, symphathetic ophthalmia, Meniere's disease, anti-neutrophil cytoplasmic antibody-associated vasculitis, Churg-Strauss syndrome, Henoch-Schonlein purpura, microscopic polyangiitis, urticarial vasculitis, and vasculitis. Examples of autoantibody-associated autoimmune conditions include gastritis and POEMS syndrome. Examples of autoantibody-associated (non-autoimmune) diseases include agammaglobulinemia, amyotrophic lateral sclerosis, Castleman's disease, cutaneous leukocytoclastic angiitis, eczema, eosinophilic gastroenteritis, erythroblastosis fetalis, fibrodysplasia ossificans progressive, hypogammaglobulinemia, idiopathic pulmonary fibrosis, IgA nephropathy, Majeed syndrome, narcolepsy, Rasmussen's encephalitis, spondyloarthropathy or Sweet's syndrome.

In some embodiments, the antigen expressed by the undesired cell comprises mesothelin, alpha-fetoprotein (ALP), BAGE, BCR-ABL, beta-catenin, beta-HCG, BrE3-antigen, BCA225, BCMA, BTAA, CA125, CA195, CA242, CA-50, CAM43, CAMEL, CAP-1, carbonic anhydrase IX, CA19-9, CA72-4, CAM 17.1, CASP-8, CCCL19, CCCL21, CD1, CD 1a, CD2, CD4, CD5, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD47, CD52, CD54, CD55, CD59, CD64, CD66a-e, CD67, CD68, CD70, CD70L, CD74, CD79a, CD79b, CD80, CD83, CD95, CD123, CD126, CD132, CD133, CD138, CD147, CD154, CDC27, CDK4, CDK4m, CDKN2A, CO-029, CTLA4, CXCR4, CXCR7, CXCL12, HIF-1a, colon-specific antigen-p (CSAp), CEACAM5) CEACAM6, c-Met, DAM, E2A-PRL, EGFR, EGFRvIII, EGP-1, EGP-2, ELF2-M, Ep-CAM, FGF, FGF-5, Flt-1, Flt-3, folate receptor, G250 antigen, Ga733VEpCAM, GAGE, gplOO, GRO-b, H4-RET, HLA-DR, HM1.24, human chorionic gonadotropin (HCG) HER2, HER3, HMGB-1, HIF-1, HSP70-2M, HST-2, HTgp-175, la, IGF-1R, IFN-g, IFN-α, IFN-b, IFN-1, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KLK2, KSA, KS-1-antigen, KS1-4, LAGE-1a, Le-Y, LDR/FUT, M344, MA-50, macrophage migration inhibitory factor (MIF), MAGE, MAGE-1, MAGE-3, MAGE-4, MAGE-5, MAGE-6, MART-1, MART-2, TRAG-3, MCP-1, MIP-1A, MIP-1B, MIF, MG7-Ag, MOV18, MUC1, MUC2, MUC3, MUC4, MUC5ac, MUC13, MUC16, MUM-1/2, MUM-3, MYL-RAR, NB/70K, Nm23H1, NuMA, NCA66, NCA95, NCA90, NY-ESO-1, p15, p16, p185erbB2, p180erbB3, PAM4 antigen, pancreatic cancer mucin, PD-1, PD-L1, PD-L2, PI5, placental growth factor, p53, PLAGL2, Pmel17 prostatic acid phosphatase, PSA, PRAME, PSMA, PlGF, ILGF, ILGF-1R, IL-6, IL-25, RCAS1, RS5, RAGE, RANTES, Ras, T101, SAGE, 5100, SLAMF7, survivin, survivin-2B, SDDCAG16, TA-90\Mac2 binding protein, TAAL6, TAC, TAG-72, TLP, tenascin, TMEFF2, TRAIL receptors, TRP-1, TRP-2, TSP-180, VEGFR, ED-B fibronectin, WT-1, 17-1A-antigen, C3, C3a, C3b, C5a, C5, bcl-2, K-ras, tumor neoantigen, a viral antigen associated with cancer, FcγRIIB, IL-12β2R, CD28, CD56, CD11c, CD66b, CD41, CD61, CD62, CD235a, CD146, CD326, or CD203c, or any combination thereof.

In some embodiments, the antigen expressed by the undesired cell is BCMA. In some embodiments, the antigen expressed by the undesired cell is PSMA.

In some embodiments, the isolated molecule is an antibody or a non-antibody molecule.

In some embodiments, the antibody comprises a first half molecule and a second half molecule, wherein the first half molecule comprises the first antigen binding domain and the second antigen binding domain and the second half molecule comprises the third antigen binding domain.

The isolated molecules and multispecific molecules comprising an antigen binding domain that specifically binds BCMA disclosed herein may be used in the treatment of multiple myeloma (MM).

In some embodiments, the multiple myeloma is a newly diagnosed multiple myeloma.

In some embodiments, the multiple myeloma is a relapsed or a refractory multiple myeloma.

In some embodiments, the multiple myeloma is a high-risk multiple myeloma.

Subjects with high-risk multiple myeloma are known to relapse early and have poor prognosis and outcome. Subjects can be classified as having high-risk multiple myeloma is they have one or more of the following cytogenetic abnormalities: t(4;14)(p16;q32), t(14;16)(q32;q23), del17p, 1qAmp, t(4;14)(p16;q32) and t(14;16)(q32;q23), t(4;14)(p16;q32) and del17p, t(14;16)(q32;q23) and del17p, or t(4;14)(p16;q32), t(14;16)(q32;q23) and del17p.

In some embodiments, the subject having the high-risk multiple myeloma has one or more chromosomal abnormalities comprising: t(4;14)(p16;q32), t(14;16)(q32;q23), del1′7p, 1qAmp, t(4;14)(p16;q32) and t(14;16)(q32;q23), t(4;14)(p16;q32) and del17p, t(14;16)(q32;q23) and del17p; or t(4;14)(p16;q32), t(14;16)(q32;q23) and del17p, or any combination thereof.

Various qualitative and/or quantitative methods may be used to determine relapse or refractory nature of the disease. Symptoms that may be associated are for example a decline or plateau of the well-being of the patient or re-establishment or worsening of various symptoms associated with solid tumors, and/or the spread of cancerous cells in the body from one location to other organs, tissues or cells.

The cytogenetic abnormalities can be detected for example by fluorescent in situ hybridization (FISH). In chromosomal translocations, an oncogene is translocated to the IgH region on chromosome 14q32, resulting in dysregulation of these genes. t(4;14)(p16;q32) involves translocation of fibroblast growth factor receptor 3 (FGFR3) and multiple myeloma SET domain containing protein (MMSET) (also called WHSC1/NSD2), and t(14;16)(q32;q23) involves translocation of the MAF transcription factor C-MAF. Deletion of 17p (del17p) involves loss of the p53 gene locus.

In some embodiments, the multiple myeloma is relapsed or refractory to treatment with the anti-CD38 antibody, lenalinomide, bortezomib, pomalidomide, carfilzomib, elotozumab, ixazomib, melphalan or thalidomide, or any combination thereof.

In some embodiments, the multiple myeloma is relapsed or refractory to treatment with the anti-CD38 antibody. In some embodiments, the multiple myeloma is relapsed or refractory to treatment with lenalinomide. In some embodiments, the multiple myeloma is relapsed or refractory to treatment with bortezomib. In some embodiments, the multiple myeloma is relapsed or refractory to treatment with pomalidomide. In some embodiments, the multiple myeloma is relapsed or refractory to treatment with carfilzomib. In some embodiments, the multiple myeloma is relapsed or refractory to treatment with elotozumab. In some embodiments, the multiple myeloma is relapsed or refractory to treatment with ixazomib. In some embodiments, the multiple myeloma is relapsed or refractory to treatment with melphalan. In some embodiments, the multiple myeloma is relapsed or refractory to treatment with or thalidomide.

The isolated molecules and multispecific molecules comprising an antigen binding domain that specifically binds PSMA disclosed herein may be used in the treatment of prostate cancer.

“Prostate cancer” is meant to include all types of cancerous growths within prostate or oncogenic processes, metastatic tissues or malignantly transformed cells, tissues, or organs, irrespective of histopathology type or stage of invasiveness.

In some embodiments, the prostate cancer is an adenocarcinoma.

In some embodiments, the prostate cancer is a metastatic prostate cancer. In some embodiments, the prostate cancer has metastasized to rectum, lymph node or bone, or any combination thereof.

In some embodiments, the prostate cancer is a relapsed or a refractory prostate cancer.

In some embodiments, the prostate cancer is a castration resistant prostate cancer.

In some embodiments, the prostate cancer is sensitive to an androgen deprivation therapy.

In some embodiments, the prostate cancer is insensitive to the androgen deprivation therapy.

In some embodiments, the subject is treatment naïve.

In some embodiments, the subject has received androgen deprivation therapy.

In some embodiments, the subject has an elevated level of prostate specific antigen (PSA). PSA is elevated in a subject when the level is typically about ≥4.0 ng/mL. In some instances, elevated PSA may refer to level off ≥3.0 ng/mL. PSA levels may also be compared to post-androgen deprivation therapy levels.

Androgen deprivation therapies include abiraterone, ketoconazole, enzalutamide, galeterone, ARN-509 and orteronel (TAK-700), or prostatectomy.

### Enrichment and Detection Methods

The isolated molecules or the isolated multispecific antibodies of the disclosure can be used to selectively enrich, isolate, separate, purify, sort, select, capture or detect CD8⁺ CTLs. The isolated molecules or the isolated multispecific antibodies of the disclosure may be utilized in a bispecific format, e.g., containing a first antigen binding domain that specifically binds CD8 and a second antigen binding domain that specifically binds the TCR complex, or they may be utilized in a format that incorporates the third antigen binding domain that specifically binds a third antigen. In some embodiments, the third antigen is an inert antigen.

The disclosure provides a method of enriching, isolating, separating, purifying, sorting, selecting, capturing or detecting a CD8+ CTL comprising:

providing a sample comprising the CD8⁺ CTL;
contacting the sample with an isolated molecule comprising a first antigen binding domain and a second antigen binding domain, wherein the first antigen binding domain specifically binds CD8 and the second antigen binding domain specifically binds a TCR complex; and
enriching, isolating, separating, purifying, sorting, selecting, capturing or detecting the CD8⁺ CTL bound to the isolated molecule.

The disclosure provides a method of enriching a CD8+ CTL comprising:

providing a sample comprising the CD8⁺ CTL;
contacting the sample with an isolated molecule comprising a first antigen binding domain and a second antigen binding domain, wherein the first antigen binding domain specifically binds CD8 and the second antigen binding domain specifically binds a TCR complex; and enriching the CD8⁺ CTL bound to the isolated molecule.

The disclosure provides a method of isolating a CD8+ CTL comprising:

providing a sample comprising the CD8⁺ CTL;
contacting the sample with an isolated molecule comprising a first antigen binding domain and a second antigen binding domain, wherein the first antigen binding domain specifically binds CD8 and the second antigen binding domain specifically binds a TCR complex; and isolating the CD8⁺ CTL bound to the isolated molecule.

The disclosure provides a method of separating a CD8+ CTL comprising:

providing a sample comprising the CD8⁺ CTL;
contacting the sample with an isolated molecule comprising a first antigen binding domain and a second antigen binding domain, wherein the first antigen binding domain specifically binds CD8 and the second antigen binding domain specifically binds a TCR complex; and
separating the CD8⁺ CTL bound to the isolated molecule.

The disclosure provides a method of purifying a CD8+ CTL comprising:

providing a sample comprising the CD8⁺ CTL;
contacting the sample with an isolated molecule comprising a first antigen binding domain and a second antigen binding domain, wherein the first antigen binding domain specifically binds CD8 and the second antigen binding domain specifically binds a TCR complex; and
purifying the CD8⁺ CTL bound to the isolated molecule.

The disclosure provides a method of sorting a CD8+ CTL comprising:

providing a sample comprising the CD8⁺ CTL;
contacting the sample with an isolated molecule comprising a first antigen binding domain and a second antigen binding domain, wherein the first antigen binding domain specifically binds CD8 and the second antigen binding domain specifically binds a TCR complex; and
sorting the CD8⁺ CTL bound to the isolated molecule.

The disclosure provides a method of selecting a CD8+ CTL comprising:

providing a sample comprising the CD8⁺ CTL;
contacting the sample with an isolated molecule comprising a first antigen binding domain and a second antigen binding domain, wherein the first antigen binding domain specifically binds CD8 and the second antigen binding domain specifically binds a TCR complex; and
selecting the CD8⁺ CTL bound to the isolated molecule.

The disclosure provides a method of capturing a CD8+ CTL comprising:

providing a sample comprising the CD8⁺ CTL;
contacting the sample with an isolated molecule comprising a first antigen binding domain and a second antigen binding domain, wherein the first antigen binding domain specifically binds CD8 and the second antigen binding domain specifically binds a TCR complex; and
capturing the CD8⁺ CTL bound to the isolated molecule.

The disclosure provides a method of detecting a CD8+ CTL comprising:

providing a sample comprising the CD8⁺ CTL;
contacting the sample with an isolated molecule comprising a first antigen binding domain and a second antigen binding domain, wherein the first antigen binding domain specifically binds CD8 and the second antigen binding domain specifically binds a TCR complex; and
detecting the CD8⁺ CTL bound to the isolated molecule.

The disclosure also provides a method of enriching, isolating, separating, purifying, sorting, selecting, capturing or detecting a CD8⁺ CTL, comprising:

contacting the CD8⁺ CTL with an isolated molecule comprising a first antigen binding domain and a second antigen binding domain, wherein the first antigen binding domain specifically binds CD8 and the second antigen binding domain specifically binds a TCR complex; and
enriching, isolating, separating, purifying, sorting, selecting, capturing or detecting the CD8⁺ CTL based on binding of the CD8⁺ CTL to the isolated molecule.

The disclosure also provides a method of enriching a CD8⁺ CTL, comprising:

contacting the CD8⁺ CTL with an isolated molecule comprising a first antigen binding domain and a second antigen binding domain, wherein the first antigen binding domain specifically binds CD8 and the second antigen binding domain specifically binds a TCR complex; and
enriching the CD8⁺ CTL based on binding of the CD8⁺ CTL to the isolated molecule.

The disclosure also provides a method of isolating a CD8⁺ CTL, comprising:

contacting the CD8⁺ CTL with an isolated molecule comprising a first antigen binding domain and a second antigen binding domain, wherein the first antigen binding domain specifically binds CD8 and the second antigen binding domain specifically binds a TCR complex; and
isolating the CD8⁺ CTL based on binding of the CD8⁺ CTL to the isolated molecule.

The disclosure also provides a method of separating a CD8⁺ CTL, comprising: contacting the CD8⁺ CTL with an isolated molecule comprising a first antigen binding domain and a second antigen binding domain, wherein the first antigen binding domain specifically binds CD8 and the second antigen binding domain specifically binds a TCR complex; and separating the CD8⁺ CTL based on binding of the CD8⁺ CTL to the isolated molecule.

The disclosure also provides a method of purifying or detecting a CD8⁺ CTL, comprising:
contacting the CD8⁺ CTL with an isolated molecule comprising a first antigen binding domain and a second antigen binding domain, wherein the first antigen binding domain specifically binds CD8 and the second antigen binding domain specifically binds a TCR complex; and
   purifying the CD8⁺ CTL based on binding of the CD8⁺ CTL to the isolated molecule.

The disclosure also provides a method of a CD8⁺ CTL, comprising:

contacting the CD8⁺ CTL with an isolated molecule comprising a first antigen binding domain and a second antigen binding domain, wherein the first antigen binding domain specifically binds CD8 and the second antigen binding domain specifically binds a TCR complex; and
sorting the CD8⁺ CTL based on binding of the CD8⁺ CTL to the isolated molecule.

The disclosure also provides a method of selecting a CD8⁺ CTL, comprising:

contacting the CD8⁺ CTL with an isolated molecule comprising a first antigen binding domain and a second antigen binding domain, wherein the first antigen binding domain specifically binds CD8 and the second antigen binding domain specifically binds a TCR complex; and
selecting the CD8⁺ CTL based on binding of the CD8⁺ CTL to the isolated molecule.

The disclosure also provides a method of capturing a CD8⁺ CTL, comprising:

contacting the CD8⁺ CTL with an isolated molecule comprising a first antigen binding domain and a second antigen binding domain, wherein the first antigen binding domain specifically binds CD8 and the second antigen binding domain specifically binds a TCR complex; and
capturing the CD8⁺ CTL based on binding of the CD8⁺ CTL to the isolated molecule.

The disclosure also provides a method of detecting a CD8⁺ CTL, comprising:

contacting the CD8⁺ CTL with an isolated molecule comprising a first antigen binding domain and a second antigen binding domain, wherein the first antigen binding domain specifically binds CD8 and the second antigen binding domain specifically binds a TCR complex; and
detecting the CD8⁺ CTL based on binding of the CD8⁺ CTL to the isolated molecule.

In some embodiments, the sample is a blood sample or a tissue sample.

In some embodiments, the method is conducted in suspension or on a solid support.

In some embodiments, the method is conducted using beads, microfluidics, fluorescent cell sorting, chips, columns or surfaces.

In some embodiments, the isolated molecule further comprises a third antigen binding domain that specifically binds a third antigen.

In some embodiments, the first antigen binding domain, the second antigen binding domain or the third antigen binding domain comprises a scFv, a Fab, a Fab′, a F(ab′)₂, a Fd, a Fv, a dAb, a VHH, a VH, a VL, a non-antibody scaffold, or fragments thereof.

In some embodiments, the isolated molecule comprises: a first polypeptide comprising, from N- to C-terminus, the second antigen binding domain comprising the scFv, a VH that is capable of specifically binding CD8, a CH1 domain, a hinge, a CH2 domain and a CH3 domain; a second polypeptide comprising, from N- to C-terminus, a VL that is capable of specifically binding CD8 and a CL domain; and a third polypeptide comprising, from N- to C-terminus, the third antigen binding domain comprising the scFv and a Fc or a fragment of the Fc.

In some embodiments, the isolated molecule comprises: a first polypeptide comprising, from N- to C-terminus, a VH that is capable of specifically binding CD8, a CH1 domain, a hinge, a CH2 domain and a CH3 domain; a second polypeptide comprising, from N- to C-terminus, a VL that is capable of specifically binding CD8, a CL domain and the second antigen binding domain comprising the scFv; and a third polypeptide comprising, from N- to C-terminus, the third antigen binding domain comprising the scFv and a Fc or a fragment of the Fc.

In some embodiments, the isolated molecule comprises: a first polypeptide comprising, from N- to C-terminus, a VH that is capable of specifically binding CD8, a CH1 domain, a hinge, a CH2 domain, a CH3 domain and the second antigen binding domain comprising the scFv; a second polypeptide comprising, from N- to C-terminus, a VL that is capable of specifically binding CD8 and a CL domain; and a third polypeptide comprising, from N- to C-terminus, the third antigen binding domain comprising the scFv and a Fc or a fragment of the Fc.

In some embodiments, the first antigen binding domain comprising the Fab, the second antigen binding domain comprising the scFv or the third antigen binding domain comprising the scFv is conjugated to the Fc or the fragment of the Fc, to the VH that is capable of specifically biding CD8, to the CL domain or to the CH3 domain via a linker.

In some embodiments, the linker comprises a polypeptide of SEQ ID NOs: 2183-2290.

In some embodiments, the fragment of the Fc comprises a CH2 domain and a CH3 domain.

In some embodiments, the Fc, the CH2 domain or the CH3 domain is an IgG1, IgG2, IgG3 or IgG4 isotype.

In some embodiments, the second antigen binding domain specifically binds CD3, TCRα chain, TCRβ chain, TCRγ chain or TCRδ chain, or any combination thereof.

In some embodiments, the TCRβ chain comprises TCRVB17.

In some embodiments, CD3 comprises CD3ε, CD3γ, CD3δ or CD3ζ.

In some embodiments, the second antigen binding domain that specifically binds CD3 comprises the HCDR1 of SEQ ID NO: 2291, the HCDR2 of SEQ ID NO: 2292, the HCDR3 of SEQ ID NO: 2293, the LCDR1 of SEQ ID NO: 2294, the LCDR2 of SEQ ID NO: 2295 and the LCDR3 of SEQ ID NO: 2296.

In some embodiments, the second antigen binding domain that specifically binds CD3 comprises the VH of SEQ ID NO: 2297 and the VL of SEQ ID NO: 2298.

In some embodiments, the first antigen binding domain comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

In some embodiments, the first antigen binding domain comprises the VH of SEQ ID NO: 2313 and the VL of SEQ ID NO: 2314.

In some embodiments, the isolated molecule is an antibody or a non-antibody molecule.

In some embodiments, the antibody comprises a first half molecule and a second half molecule, wherein the first half molecule comprises the first antigen binding domain and the second antigen binding domain and the second half molecule comprises the third antigen binding domain.

Enrichment, isolation, separation, purification, sorting, selecting, capturing or detecting, or any combination thereof can be done using known technologies such as bead, microfluidics, solid support, columns etc. In general the isolated molecule of the disclosure, when bound to the CD8⁺ CTL may be separated or visualized using known methods.

The following examples are provided to further describe some of the embodiments disclosed herein. The examples are intended to illustrate, not to limit, the disclosed embodiments.

### EXAMPLES

### Example 1: Design and Generation of Trispecific Molecules Specifically Engaging Cd8⁺ CTLS

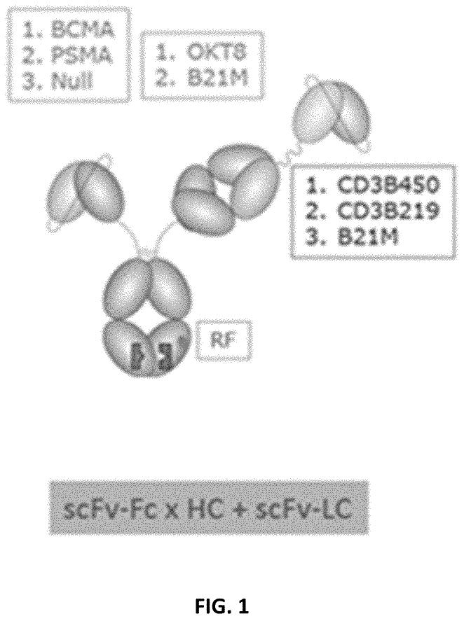

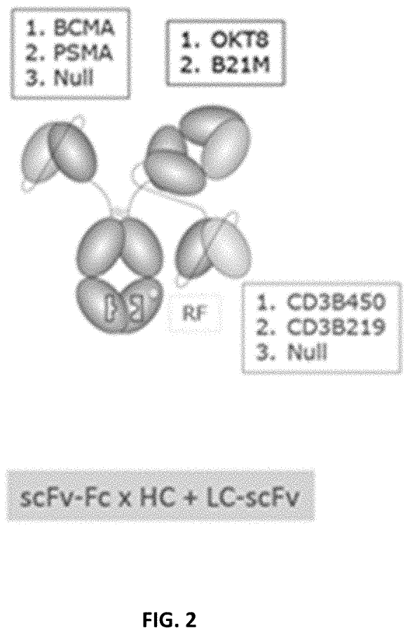

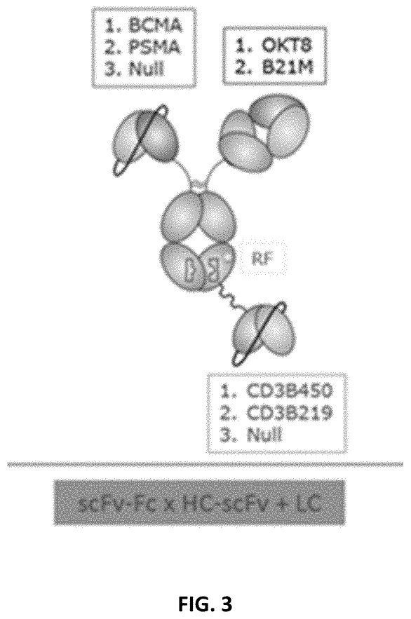

The approach to specifically engage CD8⁺ CTLs was to design and test multispecific molecules having a CD3 binding domain of various affinities, an agonistic CD8+ binding domain and a tumor associated antigen (TAA) binding domain and tailor the binding affinities within the range that would result in CD8⁺ T cell activation and tumor cell killing only in instances when co-engagement of CD3 and CD8 occurred. Towards that end, CD3 binding domains CD2B219 and CD3B450 were incorporated into a trispecific antibody together with OKT8, an agonistic CD8 binding antibody and a domain that binds the TAA. BCMA and PSMA binding domains were used to target the trispecific molecules to tumors. FIG. 1, FIG. 2 and FIG. 3 show the designed protein formats used in the study. In the Protein Format 1 (FIG. 1), the TAA binding arm was incorporated as a scFv coupled to a Fc (HC1_scFv), the CD8 binding arm was incorporated as a HC/LC chain (HC2 N-term and LC2 2^{nd} N-term), and the CD3 binding arm was incorporated as a scFv attached to the N-terminus of the CD8 binding HC (LC2 1^{st} N-term). In the Protein Format 2 (FIG. 2), the TAA binding arm was incorporated as a scFv coupled to the Fc (HC1_scFv), the CD8 binding arm was incorporated as a HC/LC chain (HC2 N-term and LC2 1^{st} N-term), and the CD3 binding arm was incorporated as a scFv attached to the C-terminus of the CD8 binding LC (LC2 C-term). In the Protein Format 3 (FIG. 3), the TAA binding arm was incorporated as a scFv coupled to the Fc (HC1_scFv), the CD8 binding arm was incorporated as a HC/LC chain (HC2 N-term and LC1 1^{st} N-term), and the CD3 binding arm was incorporated as a scFv attached to the C-terminus of the CD8 binding HC (HC2 C-term). To evaluate differences resulting from engagement of either CD3 or CD8 alone or co-engagement of CD3 and CD8, corresponding constructs were generated in which either the CD3 or the CD8 binding domain was replaced by the inert arm (RSV binding domain B21M) or not included at all (null). In some constructs, the TAA binding domain was excluded from the design.

The CD3 binding domain used were the VH/VL domains of CD3B219 or CD3B450 and the CD8 binding domain used were the VH/VL domain of OKT8. The amino acid sequences of the various domains are shown in Table 4. CD3B219 is considered a high affinity (low K_{D}) binder and CD3B450 is considered a low affinity (high K_{D}) binder. The K_{D} of CD3B219 was about 8 mM and the K_{D} of CD3B450 was about 80 nM for binding to CD3. The CD8 binding domain used were the VL/VL domains of OKT8. The amino acid sequences of OKT8 CDRs and VH/VL domains are shown in Table 5.

The trispecific molecules were cloned, expressed and purified using standard methods. To promote HC/HC heterodimerization, knob-in-hole mutations were introduced in the heavy chains.

TABLE 4

| CD3 | | | SEQ |
|---|---|---|---|
| binding | | | ID |
| domain | Region | Amino acid sequence | NO: |
| CD3B450 | HCDR1 | NNNAAWS | 2291 |
| | HCDR2 | RTYYRSKWLYDYAVSVKS | 2292 |
| | HCDR3 | GYSSSFDY | 2293 |
| | LCDR1 | TGTSSNIGTYKFVS | 2294 |
| | LCDR2 | EVSKRPS | 2295 |
| | LCDR3 | VSYAGSGTLL | 2296 |
| | VH | QVQLQQSGPGLVKPSQTLSLTCAI | 2297 |
| | | SGDSVFNNNAAWSWIRQSPSRGLE | |
| | | WLGRTYYRSKWLYDYAVSVKSRIT | |
| | | INPDTSKNQFSLQLNSVTPEDTAV | |
| | | YYCARGYSSSFDYWGQGTLVTVSS | |
| | VL | QSALTQPASVSGSPGQSITISCTG | 2298 |
| | | TSSNIGTYKFVSWYQQHPGKAPKV | |
| | | MIYEVSKRPSGVSNRFSGSKSGNT | |
| | | ASLTISGLQAEDEADYYCVSYAGS | |
| | | GTLLFGGGTKLTVL | |
| CD3B219 | HCDR1 | TYAMN | 2299 |
| | HCDR2 | RIRSKYNNYATYYAASVKG | 2300 |
| | HCDR3 | HGNFGNSYVSWFAY | 2301 |
| | LCDR1 | RSSTGAVTTSNYAN | 2302 |
| | LCDR2 | GTNKRAP | 2303 |
| | LCDR3 | ALWYSNLWV | 2304 |
| | VH | EVQLVESGGGLVQPGGSLRLSCAA | 2305 |
| | | SGFTFNTYAMNWVRQAPGKGLEWV | |
| | | ARIRSKYNNYATYYAASVKGRFTI | |
| | | SRDDSKNSLYLQMNSLKTEDTAVY | |
| | | YCARHGNFGNSYVSWFAYWGQGTL | |
| | | VTVSS | |
| | VL | QTVVTQEPSLTVSPGGTVTLTCRS | 2306 |
| | | STGAVTTSNYANWVQQKPGQAPRG | |
| | | LIGGTNKRAPGTPARFSGSLLGGK | |
| | | AALTLSGVQPEDEAEYYCALWYSN | |
| | | LWVFGGGTKLTVL | |

TABLE 5

| CD8 | | | SEQ |
|---|---|---|---|
| binding | | | ID |
| domain | Region | Amino acid sequence | NO: |
| OKT8 | HCDR1 | DTYIH | 2307 |
| | HCDR2 | RIDPANDNTLYASKFQG | 2308 |
| | HCDR3 | GYGYYVFDH | 2309 |
| | LCDR1 | RTSRSISQYLA | 2310 |
| | LCDR2 | SGSGS | 2311 |
| | LCDR3 | QQHNENPLT | 2312 |
| | VH | EVQLQQSGAELVKPGASVKLSCTAS | 2313 |
| | | GFNIKDTYIHFVRQRPEQGLEWIGR | |
| | | IDPANDNTLYASKFQGKATITADTS | |
| | | SNTAYMHLCSLTSGDTAVYYCGRGY | |
| | | GYYVFDHWGQGTTLTVSS | |
| | VL | DVQINQSPSFLAASPGETITINCRT | 2314 |
| | | SRSISQYLAWYQEKPGKTNKLLIYS | |
| | | GSTLQSGIPSRFSGSGSGTDFTLTI | |
| | | SGLEPEDFAMYYCQQHNENPLTFGA | |
| | | GTKLELR | |

The specific constructs generated incorporating CD3, CD8 and BCMA binding domains are shown in Table 6. Table 6 constructs 1-12 were engineered as Protein Format 1, constructs 13-17 and 31-36 were engineered as Protein Format 2, and constructs 19-30 were engineered as Protein Format 3. The specific constructs generated incorporating CD3, CD8 and PSMA binding domains are shown in Table 7. Table 7 constructs P3-P5, P15-P17, P21-P23 and P33-P35 were engineered as Protein Format 1, constructs P6-P8, P12-P14, P24-P26 and P30-P32 were engineered as Protein Format 2, and constructs P1, P2, P9-P11, P18-P20, P270P29 and P36 were engineered as Protein Format 3.

TABLE 6

| Construct | | HC2 | HC2 | LC2 | LC2 | LC2 |
|---|---|---|---|---|---|---|
| number | HC1_scFv | (N-term) | (C-term) | (1st N-term) | (2nd N-term) | (C-term) |
| 1 | BCMA-scFv | OKT8-Fab- | n/a | CD3B450-LH- | OKT8-LC | n/a |
| | | RF | | scFv | | |
| 2 | BCMA-scFv | OKT8-Fab- | n/a | CD3B219-LH- | OKT8-LC | n/a |
| | | RF | | scFv | | |
| 3 | BCMA-scFv | OKT8-Fab- | n/a | null-scFv | OKT8-LC | n/a |
| | | RF | | | | |
| 4 | BCMA-scFv | B21M-Fab- | n/a | CD3B450-LH- | B21M-LC | n/a |
| | | RF | | scFv | | |
| 5 | BCMA-scFv | B21M-Fab- | n/a | CD3B219-LH- | B21M-LC | n/a |
| | | RF | | scFv | | |
| 6 | BCMA-scFv | B21M-Fab- | n/a | null-scFv | B21M-LC | n/a |
| | | RF | | | | |
| 7 | null-scFv | OKT8-Fab- | n/a | CD3B450-LH- | OKT8-LC | n/a |
| | | RF | | scFv | | |
| 8 | null-scFv | OKT8-Fab- | n/a | CD3B219-LH- | OKT8-LC | n/a |
| | | RF | | scFv | | |
| 9 | null-scFv | OKT8-Fab- | n/a | null-scFv | OKT8-LC | n/a |
| | | RF | | | | |
| 10 | null-scFv | B21M-Fab- | n/a | CD3B450-LH- | B21M-LC | n/a |
| | | RF | | scFv | | |
| 11 | null-scFv | B21M-Fab- | n/a | CD3B219-LH- | B21M-LC | n/a |
| | | RF | | scFv | | |
| 12 | null-scFv | B21M-Fab- | n/a | null-scFv | B21M-LC | n/a |
| | | RF | | | | |
| 13 | BCMA-scFv | OKT8-Fab- | n/a | OKT8-LC | n/a | CD3B450-LH- |
| | | RF | | | | scFv |
| 14 | BCMA-scFv | OKT8-Fab- | n/a | OKT8-LC | n/a | CD3B219-LH- |
| | | RF | | | | scFv |
| 15 | BCMA-scFv | OKT8-Fab- | n/a | OKT8-LC | n/a | null-scFv |
| | | RF | | | | |
| 16 | BCMA-scFv | B21M-Fab- | n/a | B21M-LC | n/a | CD3B450-LH- |
| | | RF | | | | scFv |
| 17 | BCMA-scFv | B21M-Fab- | n/a | B21M-LC | n/a | CD3B219-LH- |
| | | RF | | | | scFv |
| 18 | BCMA-scFv | B21M-Fab- | n/a | B21M-LC | n/a | null-scFv |
| | | RF | | | | |
| 34 | null-scFv | OKT8-Fab- | n/a | OKT8-LC | n/a | CD3B450-LH- |
| | | RF | | | | scFv |
| 35 | null-scFv | OKT8-Fab- | n/a | OKT8-LC | n/a | CD3B219-LH- |
| | | RF | | | | scFv |
| 36 | null-scFv | OKT8-Fab- | n/a | OKT8-LC | n/a | null-scFv |
| | | RF | | | | |
| 31 | null-scFv | B21M-Fab- | n/a | B21M-LC | n/a | CD3B450-LH- |
| | | RF | | | | scFv |
| 32 | null-scFv | B21M-Fab- | n/a | B21M-LC | n/a | CD3B219-LH- |
| | | RF | | | | scFv |
| 33 | null-scFv | B21M-Fab- | n/a | B21M-LC | n/a | null-scFv |
| | | RF | | | | |
| 19 | BCMA-scFv | OKT8-Fab- | CD3B450-LH- | OKT8-LC | n/a | n/a |
| | | RF | scFv | | | |
| 20 | BCMA-scFv | OKT8-Fab- | CD3B219-LH- | OKT8-LC | n/a | n/a |
| | | RF | scFv | | | |
| 21 | BCMA-scFv | OKT8-Fab- | null-scFv | OKT8-LC | n/a | n/a |
| | | RF | | | | |
| 22 | BCMA-scFv | B21M-Fab- | CD3B450-LH- | B21M-LC | n/a | n/a |
| | | RF | scFv | | | |
| 23 | BCMA-scFv | B21M-Fab- | CD3B219-LH- | B21M-LC | n/a | n/a |
| | | RF | scFv | | | |
| 24 | BCMA-scFv | B21M-Fab- | null-scFv | B21M-LC | n/a | n/a |
| | | RF | | | | |
| 25 | null-scFv | OKT8-Fab- | CD3B450-LH- | OKT8-LC | n/a | n/a |
| | | RF | scFv | | | |
| 26 | null-scFv | OKT8-Fab- | CD3B219-LH- | OKT8-LC | n/a | n/a |
| | | RF | scFv | | | |
| 27 | null-scFv | OKT8-Fab- | null-scFv | OKT8-LC | n/a | n/a |
| | | RF | | | | |
| 28 | null-scFv | B21M-Fab- | CD3B450-LH- | B21M-LC | n/a | n/a |
| | | RF | scFv | | | |
| 29 | null-scFv | B21M-Fab- | CD3B219-LH- | B21M-LC | n/a | n/a |
| | | RF | scFv | | | |
| 30 | null-scFv | B21M-Fab- | null-scFv | B21M-LC | n/a | n/a |
| | | RF | | | | |

TABLE 7

| Construct | | HC2 | HC2 | LC2 | LC2 | LC2 |
|---|---|---|---|---|---|---|
| number | HC1_scFv | (N-term) | (C-term) | (1st N-term) | (2nd N-term) | (C-term) |
| P4 | PSMA-scFv | OKT8-Fab- | n/a | CD3B450-LH- | OKT8-LC | n/a |
| | | RF | | scFv | | |
| P3 | PSMA-scFv | OKT8-Fab- | n/a | CD3B219-LH- | OKT8-LC | n/a |
| | | RF | | scFv | | |
| P5 | PSMA-scFv | OKT8-Fab- | n/a | null-scFv | OKT8-LC | n/a |
| | | RF | | | | |
| P16 | PSMA-scFv | B21M-Fab- | n/a | CD3B450-LH- | B21M-LC | n/a |
| | | RF | | scFv | | |
| P15 | PSMA-scFv | B21M-Fab- | n/a | CD3B219-LH- | B21M-LC | n/a |
| | | RF | | scFv | | |
| P17 | PSMA-scFv | B21M-Fab- | n/a | null-scFv | B21M-LC | n/a |
| | | RF | | | | |
| P22 | null-scFv | OKT8-Fab- | n/a | CD3B450-LH- | OKT8-LC | n/a |
| | | RF | | scFv | | |
| P21 | null-scFv | OKT8-Fab- | n/a | CD3B219-LH- | OKT8-LC | n/a |
| | | RF | | scFv | | |
| P23 | null-scFv | OKT8-Fab- | n/a | null-scFv | OKT8-LC | n/a |
| | | RF | | | | |
| P34 | null-scFv | B21M-Fab- | n/a | CD3B450-LH- | B21M-LC | n/a |
| | | RF | | scFv | | |
| P33 | null-scFv | B21M-Fab- | n/a | CD3B219-LH- | B21M-LC | n/a |
| | | RF | | scFv | | |
| P35 | null-scFv | B21M-Fab- | n/a | null-scFv | B21M-LC | n/a |
| | | RF | | | | |
| P7 | PSMA-scFv | OKT8-Fab- | n/a | OKT8-LC | n/a | CD3B450-LH- |
| | | RF | | | | scFv |
| P6 | PSMA-scFv | OKT8-Fab- | n/a | OKT8-LC | n/a | CD3B219-LH- |
| | | RF | | | | scFv |
| P8 | PSMA-scFv | OKT8-Fab- | n/a | OKT8-LC | n/a | null-scFv |
| | | RF | | | | |
| P12 | PSMA-scFv | B21M-Fab- | n/a | B21M-LC | n/a | CD3B450-LH- |
| | | RF | | | | scFv |
| P13 | PSMA-scFv | B21M-Fab- | n/a | B21M-LC | n/a | CD3B219-LH- |
| | | RF | | | | scFv |
| P14 | PSMA-scFv | B21M-Fab- | n/a | B21M-LC | n/a | null-scFv |
| | | RF | | | | |
| P25 | null-scFv | OKT8-Fab- | n/a | OKT8-LC | n/a | CD3B450-LH- |
| | | RF | | | | scFv |
| P24 | null-scFv | OKT8-Fab- | n/a | OKT8-LC | n/a | CD3B219-LH- |
| | | RF | | | | scFv |
| P26 | null-scFv | OKT8-Fab- | n/a | OKT8-LC | n/a | null-scFv |
| | | RF | | | | |
| P30 | null-scFv | B21M-Fab- | n/a | B21M-LC | n/a | CD3B450-LH- |
| | | RF | | | | scFv |
| P31 | null-scFv | B21M-Fab- | n/a | B21M-LC | n/a | CD3B219-LH- |
| | | RF | | | | scFv |
| P32 | null-scFv | B21M-Fab- | n/a | B21M-LC | n/a | null-scFv |
| | | RF | | | | |
| P2 | PSMA-scFv | OKT8-Fab- | CD3B450-LH- | OKT8-LC | n/a | n/a |
| | | RF | scFv | | | |
| P1 | PSMA-scFv | OKT8-Fab- | CD3B219-LH- | OKT8-LC | n/a | n/a |
| | | RF | scFv | | | |
| P9 | PSMA-scFv | OKT8-Fab- | null-scFv | OKT8-LC | n/a | n/a |
| | | RF | | | | |
| P11 | PSMA-scFv | B21M-Fab- | CD3B450-LH- | B21M-LC | n/a | n/a |
| | | RF | scFv | | | |
| P10 | PSMA-scFv | B21M-Fab- | CD3B219-LH- | B21M-LC | n/a | n/a |
| | | RF | scFv | | | |
| P18 | PSMA-scFv | B21M-Fab- | null-scFv | B21M-LC | n/a | n/a |
| | | RF | | | | |
| P20 | null-scFv | OKT8-Fab- | CD3B450-LH- | OKT8-LC | n/a | n/a |
| | | RF | scFv | | | |
| P19 | null-scFv | OKT8-Fab- | CD3B219-LH- | OKT8-LC | n/a | n/a |
| | | RF | scFv | | | |
| P27 | null-scFv | OKT8-Fab- | null-scFv | OKT8-LC | n/a | n/a |
| | | RF | | | | |
| P29 | null-scFv | B21M-Fab- | CD3B450-LH- | B21M-LC | n/a | n/a |
| | | RF | scFv | | | |
| P28 | null-scFv | B21M-Fab- | CD3B219-LH- | B21M-LC | n/a | n/a |
| | | RF | scFv | | | |
| P36 | null-scFv | B21M-Fab- | null-scFv | B21M-LC | n/a | n/a |
| | | RF | | | | |

### Example 2: Co-Engagement of CD3 and CD8 Results in Tumor Cell Death and Activation of T Cells

All constructs were tested for their ability to mediate tumor cell death and to activate T cells using known methods.

Table 8 shows the results of % tumor cell death and % T cell activation (as assessed by % CD25⁺ live T cells) of trispecific BCMA×CD3×CD8 antibodies and controls. Table 9 shows the results of % tumor cell death and % T cell activation of trispecific PSMA×CD3×CD8 antibodies and controls. As is shown in Table 8, constructs with low affinity CD3 binding domain mediated tumor cell death and T cell activation only via co-engagement with CD8 in the context of multispecific CD3×CD8×BCMA antibodies (construct number 1, 13, 19). Constructs with high affinity CD3 binding domain mediated tumor cell death and T cell activation without co-engagement with CD8 (constructs 15, 17, 23). Further, constructs with high affinity CD3 binding domain and CD8 binding domain without TAA binding domain were able to mediate tumor cell killing and to activate T cells (Table 8, construct 8, 35 and Table 9, constructs P21 and P24). Similarly, as is shown in Table 9, trispecific antibodies binding PSMA with high affinity CD3 domains were able to mediate tumor cell killing and T cell activation only in the presence of CD8 co-engagement. Table 10 and Table 11 shows cytokine production by T cells contacted with BCMA×CD3×CD8 trispecific antibodies or controls and Table 12 and Table 13 show cytokine production by T cells contacted with PSMA×CD3×CD8 trispecifc antibodies or controls as shown in the Tables. In general, cytokine release, tumor killing and T-cell activation by T cells appeared comparable. Overall data indicated that the trispecific constructs with CD8 antibody plus high affinity CD3 binding domain CD3B450 appeared to be weaker in releasing IFNγ than the constructs with CD8 antibody and the high affinity CD3 binding domain CD3B219. The null controls with no TAA but with CD8 and CD3 domains appeared to show some very weak cytokine activity. Overall IFNγ, IL-10 and TNFα levels appeared to be released at higher levels than the rest of the cytokines from the panel.

Cytotoxicity was measured in a real-time cell analyzer xCELLigence (Roche) using adherent tumor cell lines as target cells. All experiments were performed using the respective target cell culturing media. Fifty microliters of medium was added to E-Plates 96 (Roche, Grenzach-Wyhlen, Germany) for measurement of background values. Target cells used in the experiments include C4-2B, LnCap MM1R, H929 tumor cell lines. Target cells were seeded in an additional 100 μl medium at a density of around 10,000 cells per well. Suitable cell densities were determined by previous titration experiments. Cell attachment was monitored using the RTCA SP (Roche) instrument and the RTCA software Version 1.1 (Roche) until the plateau phase was reached. T cells were added at variant dosages of trispecific antibodies. Upon addition of effector cells, impedance measurements were performed every 15 min for up to 81 h. All experiments were performed in triplicates. Changes in electrical impedance were expressed as a dimensionless cell index (CI) value, which derives from relative impedance changes corresponding to cellular coverage of the electrode sensors, normalized to baseline impedance values with medium only. To analyze the acquired data, CI values were exported, and percentage of lysis was calculated in relation to the control cells lacking any effector T cells. The percentage of cytolysis is readily calculated using a simple formula: Percentage of cytolysis=((Cell Index no effector−Cell Index effector)/Cell Index no effector)×100. Cytotoxicity of the T cells was also tested by using the IncuCyte zoom living cell imaging system. Co-culture was set up the same as the above in xCELLigence assay. images were taken every 30 min and the number of dead cells was quantified.

The Intellicyt human T cell activation and cytokine profiling kit was applied for T cell activation and cytokine profile. Briefly, T cells were cocultured with prostate tumor cells at an effector to target cells ratio (E:T ratio) of 1 to 1 in 96-well round bottom plate in 200 ul RPMI complete media. The trispecific antibodies were co-cultured and 24 hr later, T cell activation was assessed by the TCA kit from a 30 ul cell/supernatant mixture sample following the protocol. Samples were acquired on the Intellicyt iQue Screener PLUS. Standard curves to quantitate the levels of secreted cytokines. Data were analyzed with ForeCyt software.

TABLE 8

| | | | | | % Tumor cell death | | % CD25 +ve Live T-cells | |
|---|---|---|---|---|---|---|---|---|
| Construct | Protein | Domains present | | | nM | Max. | nM | Max. |
| number | Format | TAA | CD3 | CD8 | EC₅₀ | Activity | EC₅₀ | Activity |
| Control | | | | | 0.08 | 57.63 | 0.18 | 71.83 |
| 1 | 1 | BCMA | LA | P | 0.44 | 73.94 | 0.8 | 72.85 |
| 13 | 2 | BCMA | LA | P | 0.4 | 70.64 | 1.54 | 71.09 |
| 19 | 3 | BCMA | LA | P | 0.08 | 69.59 | 0.4 | 72.25 |
| 4 | 1 | BCMA | LA | A | >10.00 | 50.38 | 6.99 | 54.74 |
| 16 | 2 | BCMA | LA | A | >10.00 | 0.89 | >10.00 | 3.88 |
| 22 | 3 | BCMA | LA | A | 7.42 | 52.49 | 5.02 | 56.05 |
| 3 | 1 | BCMA | A | P | >10.00 | −0.31 | >10.00 | 4.18 |
| 15 | 2 | BCMA | A | P | >10.00 | 1.18 | >10.00 | 4.33 |
| 21 | 3 | BCMA | A | P | >10.00 | 10.21 | >10.00 | 17.4 |
| 6 | 1 | BCMA | A | A | >10.00 | −1.25 | >10.00 | 4.39 |
| 18 | 2 | BCMA | A | A | >10.00 | −0.25 | >10.00 | 3.78 |
| 24 | 3 | BCMA | A | A | >10.00 | 0.96 | >10.00 | 3.74 |
| 7 | 1 | none | LA | P | >10.00 | 19.52 | 3.92 | 33.77 |
| 34 | 2 | none | LA | P | >10.00 | 15.57 | >10.00 | 21.75 |
| 25 | 3 | none | LA | P | >10.00 | 7.24 | >10.00 | 13.07 |
| 10 | 1 | none | LA | A | >10.00 | −0.04 | >10.00 | 4.35 |
| 31 | 2 | none | LA | A | >10.00 | 3.96 | >10.00 | 3.25 |
| 28 | 3 | none | LA | A | >10.00 | 1.09 | >10.00 | 4.48 |
| 2 | 1 | BCMA | HA | P | 0.04 | 72.26 | 0.09 | 80.34 |
| 14 | 2 | BCMA | HA | P | 0.02 | 74.38 | 0.19 | 84.94 |
| 20 | 3 | BCMA | HA | P | 0.02 | 71.62 | 0.11 | 81.04 |
| 5 | 1 | BCMA | HA | A | 0.58 | 68.37 | 0.64 | 66.49 |
| 17 | 2 | BCMA | HA | A | 0.84 | 59.12 | 1.16 | 68.07 |
| 23 | 3 | BCMA | HA | A | 0.89 | 65.04 | 1.03 | 64.55 |
| 8 | 1 | none | HA | P | 3.22 | 22.71 | 0.17 | 44.81 |
| 35 | 2 | none | HA | P | 5.76 | 29.18 | 0.77 | 48.62 |
| 26 | 3 | none | HA | P | >10.00 | 6.45 | >10.00 | 22.41 |
| 11 | 1 | none | HA | A | >10.00 | 8.93 | >10.00 | 16.37 |
| 32 | 2 | none | HA | A | >10.00 | 1.47 | >10.00 | 4.51 |
| 29 | 3 | none | HA | A | >10.00 | 0.24 | >10.00 | 4.07 |
| 9 | 1 | none | A | P | >10.00 | −0.54 | >10.00 | 4.38 |
| 36 | 2 | none | A | P | >10.00 | 14.79 | >10.00 | 13.6 |
| 27 | 3 | none | A | P | >10.00 | 0.84 | >10.00 | 4.03 |
| 12 | 1 | none | A | A | >10.00 | 12.1 | >10.00 | 16.4 |
| 33 | 2 | none | A | A | >10.00 | −0.55 | >10.00 | 3.02 |
| 30 | 3 | none | A | A | >10.00 | 0.92 | >10.00 | 4.76 |
| Positive | | | | | 0.08 | 57.6 | 0.18 | 71.8 |
| control | | | | | | | | |
| Negative | | | | | >10.00 | 6.8 | >10.00 | 4.47 |
| control | | | | | | | | |
| (HC3B1.007) | | | | | | | | |

LA: low affinity (high K_{D});
HA: high affinity (low K_{D}),
A: absent;
P: present

TABLE 9

| | | | | | % Tumor cell death | | % CD25 +ve Live T-cells | |
|---|---|---|---|---|---|---|---|---|
| Construct | Protein | Domains present | | | nM | Max. | nM | Max. |
| number | format | TAA | CD3 | CD8 | EC50 | Activity | EC50 | Activity |
| P4 | 1 | PSMA | LA | P | 1.9 | 67.7 | 9.2 | 70.6 |
| P7 | 2 | PSMA | LA | P | 0.7 | 80.1 | 2.5 | 68.2 |
| P2 | 3 | PSMA | LA | P | 0.9 | 73.8 | 2.9 | 26.8 |
| P16 | 1 | PSMA | LA | A | >10.00 | 6.7 | >10.00 | 3.3 |
| P12 | 2 | PSMA | LA | A | >10.00 | 3.8 | >10.00 | 2.8 |
| P11 | 3 | PSMA | LA | A | >10.00 | 9.8 | >10.00 | 3.2 |
| P5 | 1 | PSMA | A | P | >10.00 | 4.7 | >10.00 | 3.9 |
| P8 | 2 | PSMA | A | P | >10.00 | 9.8 | >10.00 | 4.8 |
| P9 | 3 | PSMA | A | P | >10.00 | 13.7 | >10.00 | 3.3 |
| P17 | 1 | PSMA | A | A | >10.00 | 7.5 | >10.00 | 5.1 |
| P14 | 2 | PSMA | A | A | >10.00 | 4.7 | >10.00 | 3.8 |
| P18 | 3 | PSMA | A | A | >10.00 | 7.9 | >10.00 | 3.5 |
| P22 | 1 | none | LA | P | >10.00 | 8.9 | >10.00 | 25.4 |
| P25 | 2 | none | LA | P | >10.00 | 67.9 | >10.00 | 45.7 |
| P20 | 3 | none | LA | P | >10.00 | 9.9 | >10.00 | 3.8 |
| P34 | 1 | none | LA | A | >10.00 | 9.4 | >10.00 | 3.7 |
| P30 | 2 | none | LA | A | >10.00 | 9.5 | >10.00 | 6.3 |
| P29 | 3 | none | LA | A | >10.00 | 7.9 | >10.00 | 3.3 |
| P3 | 1 | PSMA | HA | P | 0.2 | 72.4 | 0.3 | 82.1 |
| P6 | 2 | PSMA | HA | P | 0.03 | 83.1 | 0.3 | 76.7 |
| P1 | 3 | PSMA | HA | P | 0.6 | 84.6 | >10.00 | 47.5 |
| P15 | 1 | PSMA | HA | A | >10.00 | 14.5 | 6.6 | 15.2 |
| P13 | 2 | PSMA | HA | A | >10.00 | 79.1 | >10.00 | 19.0 |
| P10 | 3 | PSMA | HA | A | >10.00 | 14.2 | >10.00 | 5.3 |
| P21 | 1 | none | HA | P | 0.2 | 67.5 | 0.2 | 60.2 |
| P24 | 2 | none | HA | P | 1.5 | 59.7 | 1.9 | 64.7 |
| P19 | 3 | none | HA | P | >10.00 | 7.6 | >10.00 | 4.7 |
| P33 | 1 | none | HA | A | >10.00 | 8.6 | >10.00 | 3.1 |
| P31 | 2 | none | HA | A | >10.00 | 13.5 | >10.00 | 7.9 |
| P28 | 3 | none | HA | A | >10.00 | 5.2 | >10.00 | 2.8 |
| P23 | 1 | none | A | P | >10.00 | 5.4 | >10.00 | 3.1 |
| P26 | 2 | none | A | P | >10.00 | 14.3 | >10.00 | 3.7 |
| P27 | 3 | none | A | P | >10.00 | 7.0 | >10.00 | 3.2 |
| P35 | 1 | none | A | A | >10.00 | 2.8 | >10.00 | 4.7 |
| P32 | 2 | none | A | A | >10.00 | 6.1 | >10.00 | 2.9 |
| P36 | 3 | none | A | A | >10.00 | 7.7 | 0.4 | 7.7 |
| Positive | | | | | 0.6 | 80.2 | 1.2 | 75.1 |
| control | | | | | | | | |
| Negative | | | | | >10.00 | 14.5 | >10.00 | 3.5 |
| control | | | | | | | | |

LA: low affinity (high K_{D});
HA: high affinity (low K_{D}),
A: absent;
P: present

TABLE 10

| Construct | Protein | Domains present | | | | | | |
|---|---|---|---|---|---|---|---|---|
| number | Format | TAA | CD3 | CD8 | IFNγ | IL-1b | IL-2 | IL-4 |
| 1 | 1 | BCMA | LA | P | 1.049 | 0.986 | 10.000 | 1.056 |
| 13 | 2 | BCMA | LA | P | 0.834 | 10.000 | 1.244 | 10.000 |
| 19 | 3 | BCMA | LA | P | 0.195 | 10.000 | 10.000 | 0.354 |
| 4 | 1 | BCMA | LA | A | 10.000 | 10.000 | 10.000 | 10.000 |
| 16 | 2 | BCMA | LA | A | 10.000 | 10.000 | 10.000 | 10.000 |
| 22 | 3 | BCMA | LA | A | 10.000 | 10.000 | 10.000 | 3.158 |
| 3 | 1 | BCMA | A | P | 10.000 | 10.000 | 10.000 | 10.000 |
| 15 | 2 | BCMA | A | P | 10.000 | 10.000 | 10.000 | 3.333 |
| 21 | 3 | BCMA | A | P | 10.000 | 10.000 | 10.000 | 10.000 |
| 6 | 1 | BCMA | A | A | 10.000 | 10.000 | 10.000 | 10.000 |
| 18 | 2 | BCMA | A | A | 10.000 | 10.000 | 10.000 | 10.000 |
| 24 | 3 | BCMA | A | A | 10.000 | 10.000 | 10.000 | 10.000 |
| 7 | 1 | none | LA | P | 10.000 | 10.000 | 10.000 | 10.000 |
| 34 | 2 | none | LA | P | 10.000 | 10.000 | 10.000 | 10.000 |
| 25 | 3 | none | LA | P | 10.000 | 0.001 | 10.000 | 10.000 |
| 10 | 1 | none | LA | A | 10.000 | 10.000 | 10.000 | 10.000 |
| 31 | 2 | none | LA | A | 10.000 | 0.004 | 10.000 | 1.111 |
| 28 | 3 | none | LA | A | 10.000 | 10.000 | 10.000 | 10.000 |
| 2 | 1 | BCMA | HA | P | 0.324 | 0.158 | 6.757 | 0.043 |
| 14 | 2 | BCMA | HA | P | 0.042 | 0.037 | 10.000 | 10.000 |
| 20 | 3 | BCMA | HA | P | 0.060 | 10.000 | 10.000 | 0.000 |
| 5 | 1 | BCMA | HA | A | 0.958 | 4.737 | 2.491 | 0.973 |
| 17 | 2 | BCMA | HA | A | 1.108 | 10.000 | 2.842 | 9.057 |
| 23 | 3 | BCMA | HA | A | 1.697 | 10.000 | 2.659 | 1.114 |
| 8 | 1 | none | HA | P | 10.000 | 10.000 | 10.000 | 0.551 |
| 35 | 2 | none | HA | P | 0.992 | 0.400 | 10.000 | 10.000 |
| 26 | 3 | none | HA | P | 10.000 | 10.000 | 10.000 | 10.000 |
| 11 | 1 | none | HA | A | 10.000 | 10.000 | 10.000 | 10.000 |
| 32 | 2 | none | HA | A | 10.000 | | 10.000 | 10.000 |
| 29 | 3 | none | HA | A | 10.000 | 10.000 | 10.000 | 10.000 |
| 9 | 1 | none | A | P | 10.000 | 10.000 | 10.000 | 10.000 |
| 36 | 2 | none | A | P | 10.000 | 10.000 | 10.000 | 10.000 |
| 27 | 3 | none | A | P | 10.000 | | 10.000 | 10.000 |
| 12 | 1 | none | A | A | 10.000 | 10.000 | 10.000 | 10.000 |
| 33 | 2 | none | A | A | 10.000 | 10.000 | 10.000 | 10.000 |
| 30 | 3 | none | A | A | 10.000 | 10.000 | 10.000 | 10.000 |
| Positive | | | | | 0.248 | 0.002 | 0.374 | 0.129 |
| Control | | | | | | | | |
| HC3B1.007 | | | | | 10.000 | 10.000 | 10.000 | 10.000 |

LA: low affinity (high K_{D});
HA: high affinity (low K_{D}),
A: absent;
P: present

TABLE 11

| Construct | Protein | Domains present | | | | | | | |
|---|---|---|---|---|---|---|---|---|---|
| number | Format | TAA | CD3 | CD8 | IL-6 | IL-8 | IL-10 | IL-13 | TNFα |
| 1 | 1 | BCMA | LA | P | 0.864 | 10.000 | 2.594 | 10.000 | 1.450 |
| 13 | 2 | BCMA | LA | P | 0.649 | 0.416 | 7.071 | 10.000 | 1.585 |
| 19 | 3 | BCMA | LA | P | 0.057 | 0.000 | 1.498 | 10.000 | 1.380 |
| 4 | 1 | BCMA | LA | A | 10.000 | 2.987 | 10.000 | 10.000 | 10.000 |
| 16 | 2 | BCMA | LA | A | 10.000 | 9.776 | 10.000 | 10.000 | 10.000 |
| 22 | 3 | BCMA | LA | A | 10.000 | 4.399 | 10.000 | 10.000 | 10.000 |
| 3 | 1 | BCMA | A | P | 10.000 | 10.000 | 10.000 | 10.000 | 10.000 |
| 15 | 2 | BCMA | A | P | 10.000 | 10.000 | 10.000 | 10.000 | 10.000 |
| 21 | 3 | BCMA | A | P | 10.000 | 10.000 | 10.000 | 10.000 | 10.000 |
| 6 | 1 | BCMA | A | A | 10.000 | 10.000 | 10.000 | 10.000 | 10.000 |
| 18 | 2 | BCMA | A | A | 10.000 | 10.000 | 10.000 | 10.000 | 10.000 |
| 24 | 3 | BCMA | A | A | 10.000 | 10.000 | 10.000 | 10.000 | 10.000 |
| 7 | 1 | none | LA | P | 10.000 | 10.000 | 10.000 | 10.000 | 10.000 |
| 34 | 2 | none | LA | P | 10.000 | 10.000 | 10.000 | 10.000 | 10.000 |
| 25 | 3 | none | LA | P | 10.000 | 10.000 | 10.000 | 10.000 | 10.000 |
| 10 | 1 | none | LA | A | 10.000 | 10.000 | 10.000 | 10.000 | 10.000 |
| 31 | 2 | none | LA | A | 10.000 | 10.000 | 10.000 | 10.000 | 10.000 |
| 28 | 3 | none | LA | A | 10.000 | 10.000 | 10.000 | 10.000 | 10.000 |
| 2 | 1 | BCMA | HA | P | 0.115 | 0.065 | 0.474 | 0.000 | 0.807 |
| 14 | 2 | BCMA | HA | P | 0.041 | 0.739 | 10.000 | 10.000 | 10.000 |
| 20 | 3 | BCMA | HA | P | 0.056 | 0.000 | 1.104 | 0.095 | 0.695 |
| 5 | 1 | BCMA | HA | A | 0.643 | 10.000 | 0.443 | 10.000 | 1.113 |
| 17 | 2 | BCMA | HA | A | 0.773 | 0.672 | 1.089 | 10.000 | 10.000 |
| 23 | 3 | BCMA | HA | A | 1.271 | 0.000 | 1.122 | 10.000 | 1.219 |
| 8 | 1 | none | HA | P | 5.135 | 0.561 | 1.404 | 10.000 | 0.994 |
| 35 | 2 | none | HA | P | 10.000 | 1.070 | 2.992 | 10.000 | 6.925 |
| 26 | 3 | none | HA | P | 10.000 | 10.000 | 10.000 | 10.000 | 10.000 |
| 11 | 1 | none | HA | A | 10.000 | 10.000 | 10.000 | 10.000 | 10.000 |
| 32 | 2 | none | HA | A | 10.000 | 10.000 | 10.000 | 10.000 | 10.000 |
| 29 | 3 | none | HA | A | 10.000 | 10.000 | 10.000 | 10.000 | 10.000 |
| 9 | 1 | none | A | P | 10.000 | 10.000 | 10.000 | 10.000 | 10.000 |
| 36 | 2 | none | A | P | 10.000 | 10.000 | 10.000 | 10.000 | 10.000 |
| 27 | 3 | none | A | P | 10.000 | 10.000 | 10.000 | 10.000 | 10.000 |
| 12 | 1 | none | A | A | 10.000 | 10.000 | 10.000 | 0.008 | 10.000 |
| 33 | 2 | none | A | A | 10.000 | 10.000 | 10.000 | | |
| 30 | 3 | none | A | A | 10.000 | 10.000 | 10.000 | 10.000 | 10.000 |
| Positive | | | | | 0.074 | 0.002 | 0.123 | 0.116 | 0.327 |
| Control | | | | | | | | | |
| Negative | | | | | 10.000 | 10.000 | 10.000 | 10.000 | 10.000 |
| control | | | | | | | | | |
| (HC3B1.007) | | | | | | | | | |

LA: low affinity (high K_{D});
HA: high affinity (low K_{D}),
A: absent;
P: present

TABLE 12

| Construct | Protein | | | | | | | |
|---|---|---|---|---|---|---|---|---|
| number | format | TAA | CD3 | CD8 | IFNγ | IL-1B | IL2 | IL4 |
| P4 | 1 | PSMA | LA | P | 5.672 | 5.350 | 10.000 | 10.000 |
| P7 | 2 | PSMA | LA | P | 4.622 | 1.670 | 10.000 | 10.000 |
| P2 | 3 | PSMA | LA | P | 10.000 | 1.537 | 10.000 | 10.000 |
| P16 | 1 | PSMA | LA | A | 10.000 | 10.000 | 10.000 | 10.000 |
| P12 | 2 | PSMA | LA | A | 10.000 | 10.000 | 10.000 | 0.041 |
| P11 | 3 | PSMA | LA | A | 10.000 | 10.000 | 10.000 | 10.000 |
| P5 | 1 | PSMA | A | P | 10.000 | 10.000 | 10.000 | 10.000 |
| P8 | 2 | PSMA | A | P | 10.000 | 10.000 | 10.000 | 10.000 |
| P9 | 3 | PSMA | A | P | 10.000 | 10.000 | 10.000 | 10.000 |
| P17 | 1 | PSMA | A | A | 10.000 | 10.000 | 10.000 | 0.370 |
| P14 | 2 | PSMA | A | A | 10.000 | 10.000 | 10.000 | 10.000 |
| P18 | 3 | PSMA | A | A | 10.000 | 10.000 | 10.000 | 3.333 |
| P22 | 1 | none | LA | P | 9.984 | 10.000 | 10.000 | 10.000 |
| P25 | 2 | none | LA | P | 8.333 | 9.076 | 10.000 | 10.000 |
| P20 | 3 | none | LA | P | 10.000 | 10.000 | 10.000 | 3.333 |
| P34 | 1 | none | LA | A | 10.000 | 10.000 | 10.000 | 10.000 |
| P30 | 2 | none | LA | A | 10.000 | 10.000 | 10.000 | 0.370 |
| P29 | 3 | none | LA | A | 10.000 | 10.000 | 9.299 | 0.370 |
| P3 | 1 | PSMA | HA | P | 0.489 | 0.267 | 10.000 | 10.000 |
| P6 | 2 | PSMA | HA | P | 0.867 | 0.085 | 10.000 | 10.000 |
| P1 | 3 | PSMA | HA | P | 9.596 | 0.263 | 10.000 | 10.000 |
| P15 | 1 | PSMA | HA | A | 10.000 | 10.000 | 10.000 | 0.370 |
| P13 | 2 | PSMA | HA | A | 10.000 | 10.000 | 10.000 | 10.000 |
| P10 | 3 | PSMA | HA | A | 10.000 | 10.000 | 10.000 | 10.000 |
| P21 | 1 | none | HA | P | 0.316 | 0.285 | 10.000 | 10.000 |
| P24 | 2 | none | HA | P | 8.126 | 5.372 | 10.000 | 10.000 |
| P19 | 3 | none | HA | P | 10.000 | 10.000 | 10.000 | 3.333 |
| P33 | 1 | none | HA | A | 10.000 | 10.000 | 10.000 | 10.000 |
| P31 | 2 | none | HA | A | 1.111 | 10.000 | 0.000 | 0.005 |
| P28 | 3 | none | HA | A | 1.111 | 10.000 | 10.000 | 0.370 |
| P23 | 1 | none | A | P | 10.000 | 10.000 | 10.000 | 10.000 |
| P26 | 2 | none | A | P | 1.111 | 0.001 | 10.000 | 10.000 |
| P27 | 3 | none | A | P | 10.000 | 10.000 | 10.000 | 1.111 |
| P35 | 1 | none | A | A | 10.000 | 10.000 | 10.000 | 10.000 |
| P32 | 2 | none | A | A | 3.333 | 10.000 | 10.000 | 0.370 |
| P36 | 3 | none | A | A | 10.000 | 10.000 | 10.000 | 0.123 |
| Negative | | | | | 10.000 | 10.000 | 10.000 | 10.000 |
| control | | | | | | | | |
| Positive | | | | | 10.000 | 1.104 | 10.000 | 10.000 |
| control | | | | | | | | |

LA: low affinity (high K_{D});
HA: high affinity (low K_{D}),
A: absent;
P: present

TABLE 13

| Construct | Protein | | | | | | | | |
|---|---|---|---|---|---|---|---|---|---|
| number | format | TAA | CD3 | CD8 | IL6 | IL8 | IL10 | IL13 | TNFα |
| P4 | 1 | PSMA | LA | P | 10.000 | 5.061 | 8.295 | 3.861 | 8.710 |
| P7 | 2 | PSMA | LA | P | 0.996 | 0.441 | 5.187 | 1.230 | 8.490 |
| P2 | 3 | PSMA | LA | P | 10.000 | 1.145 | 10.000 | 0.960 | 10.000 |
| P16 | 1 | PSMA | LA | A | 10.000 | 10.000 | 10.000 | 10.000 | 10.000 |
| P12 | 2 | PSMA | LA | A | 10.000 | 10.000 | 10.000 | 10.000 | 10.000 |
| P11 | 3 | PSMA | LA | A | 10.000 | 10.000 | 10.000 | 10.000 | 0.123 |
| P5 | 1 | PSMA | A | P | 10.000 | 10.000 | 10.000 | 10.000 | 10.000 |
| P8 | 2 | PSMA | A | P | 0.002 | 10.000 | 10.000 | 0.002 | 10.000 |
| P9 | 3 | PSMA | A | P | 10.000 | 10.000 | 10.000 | 10.000 | 0.000 |
| P17 | 1 | PSMA | A | A | 3.333 | 10.000 | 10.000 | 10.000 | 3.333 |
| P14 | 2 | PSMA | A | A | 10.000 | 10.000 | 10.000 | 10.000 | 0.123 |
| P18 | 3 | PSMA | A | A | 3.333 | 10.000 | 3.333 | 1.111 | 0.000 |
| P22 | 1 | none | LA | P | 10.000 | 8.820 | 10.000 | 10.000 | 10.000 |
| P25 | 2 | none | LA | P | 10.000 | 3.333 | 8.925 | 10.000 | 10.000 |
| P20 | 3 | none | LA | P | 10.000 | 0.000 | 10.000 | 1.111 | 0.000 |
| P34 | 1 | none | LA | A | 10.000 | 10.000 | 10.000 | 10.000 | 10.000 |
| P30 | 2 | none | LA | A | 10.000 | 10.000 | 10.000 | 0.466 | 10.000 |
| P29 | 3 | none | LA | A | 10.000 | 10.000 | 10.000 | 10.000 | 10.000 |
| P3 | 1 | PSMA | HA | P | 1.151 | 0.000 | 0.502 | 0.000 | 0.700 |
| P6 | 2 | PSMA | HA | P | 0.888 | 0.000 | 2.192 | 0.050 | 1.453 |
| P1 | 3 | PSMA | HA | P | 10.000 | 0.000 | 10.000 | 0.177 | 10.000 |
| P15 | 1 | PSMA | HA | A | 10.000 | 7.143 | 10.000 | 10.000 | 10.000 |
| P13 | 2 | PSMA | HA | A | 10.000 | 3.498 | 10.000 | 10.000 | 10.000 |
| P10 | 3 | PSMA | HA | A | 10.000 | 10.000 | 10.000 | 10.000 | 10.000 |
| P21 | 1 | none | HA | P | 0.373 | 0.030 | 0.385 | 0.260 | 1.395 |
| P24 | 2 | none | HA | P | 10.000 | 1.093 | 10.000 | 1.633 | 10.000 |
| P19 | 3 | none | HA | P | 10.000 | 10.000 | 10.000 | 10.000 | 10.000 |
| P33 | 1 | none | HA | A | 10.000 | 10.000 | 10.000 | 0.000 | 10.000 |
| P31 | 2 | none | HA | A | 3.333 | 0.000 | 3.333 | 10.000 | 10.000 |
| P28 | 3 | none | HA | A | 10.000 | 10.000 | 10.000 | 10.000 | 0.370 |
| P23 | 1 | none | A | P | 10.000 | 10.000 | 10.000 | 10.000 | 10.000 |
| P26 | 2 | none | A | P | 10.000 | 10.000 | 10.000 | 10.000 | 1.111 |
| P27 | 3 | none | A | P | 10.000 | 10.000 | 3.333 | 3.333 | 0.000 |
| P35 | 1 | none | A | A | 10.000 | 10.000 | 10.000 | 10.000 | 10.000 |
| P32 | 2 | none | A | A | 3.333 | 10.000 | 0.370 | 10.000 | 10.000 |
| P36 | 3 | none | A | A | 10.000 | 10.000 | 10.000 | 10.000 | 0.370 |
| Negative | | | | | 10.000 | 3.333 | 10.000 | 4.617 | 10.000 |
| control | | | | | | | | | |
| Positive | | | | | 1.793 | 0.370 | 3.440 | 0.528 | 2.967 |
| control | | | | | | | | | |

LA: low affinity (high K_{D});
HA: high affinity (low K_{D}),
A: absent;
P: present

### Example 3: Low Affinity CD3 Multispecifics Paired with CD8 Binders Show Selective Activation of CD8 T Cells and Reduced Anti-Inflammatory Cytokine Release

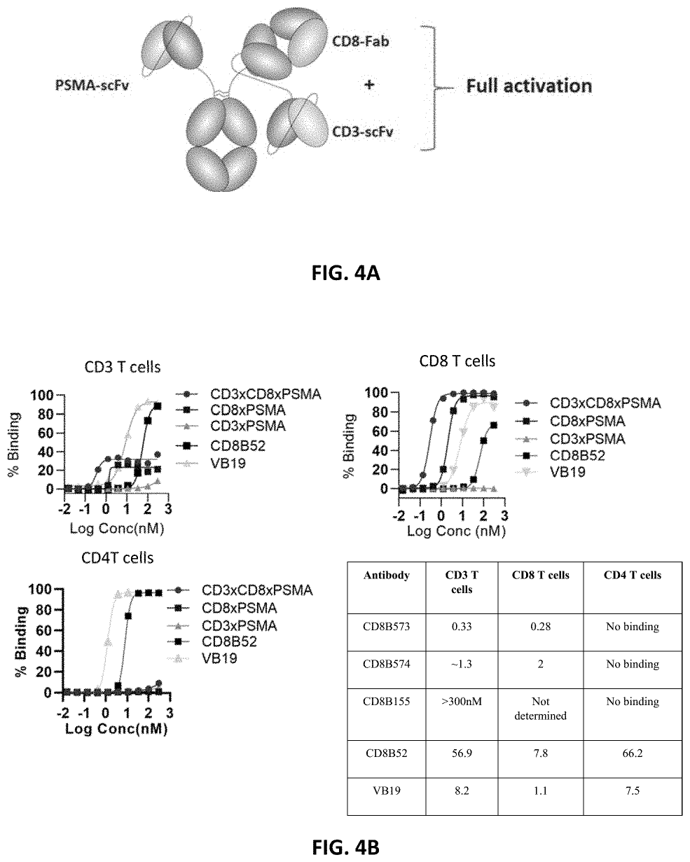

Trispecific PSMA×CD3×CD8 antibodies were constructed as shown in FIG. 4A. Pan T cells were isolated from the peripheral blood mononuclear cells (PBMCs) of healthy volunteers and stained with the test multispecifics at room temperature for 30 min followed by detection using an anti-human IgG antibody and staining with anti-human CD3, CD4 and CD8 antibodies. Binding affinity was determined using the secondary antibody-stained samples as negative controls. As shown in FIG. 4B and Table 14, low affinity CD3 multispecifics paired with CD8 binders show higher selective binding to CD8 T cells compared to the controls.

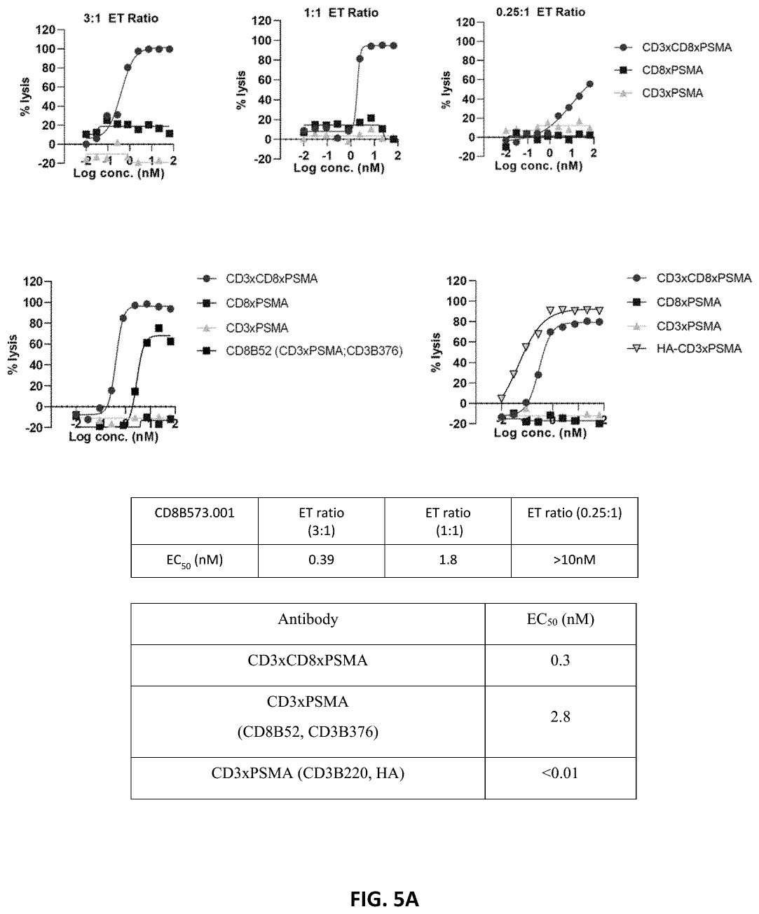

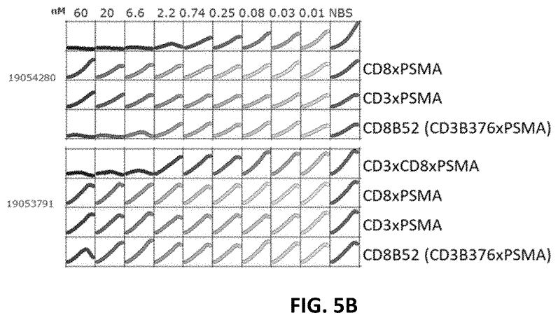

Low affinity CD3 multispecifics paired with CD8 binders demonstrated superior effects in cytotoxicity assays on C4-2B cells (target) and PBMCs (effector) (see FIG. 5A and FIG. 5B).

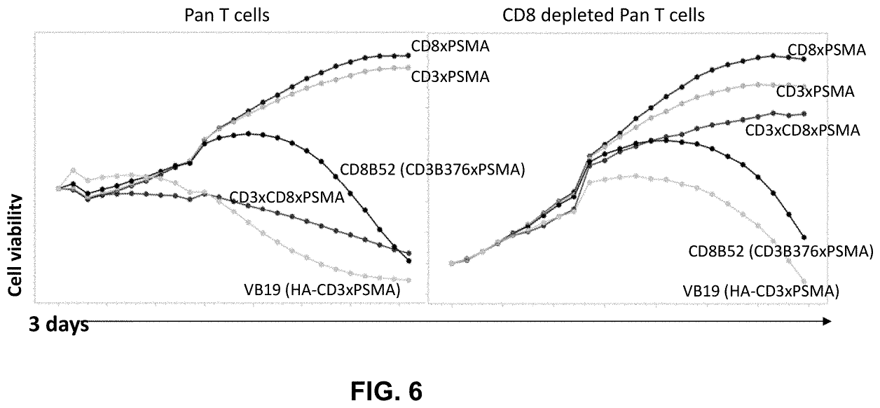

Low affinity CD3 multispecifics paired with CD8 binders were tested for potent cytotoxicity against target cell lines in a CD8 T cell dependent manner. PBMCs of healthy volunteers were either depleted of CD8 T cells or used as such. CD8 depleted and non depleted PBMCs were cocultured with C4-2B target cells as a 1:1 effector to target ratio (CD3 to target cells) for 72 hrs in the presence of the test multispecifics. Cytotoxicity was monitored using the Incucyte automated live cell analysis system and EC50 values were calculated after normalizing to no multispecific containing wells. As shown in FIG. 6, C4-2B target cells liability is high in the CD8 T cells depletion group indicating that low affinity CD3 multispecifics paired with CD8 binders show potent cytotoxicity against target cell lines in a CD8 T cell dependent manner.

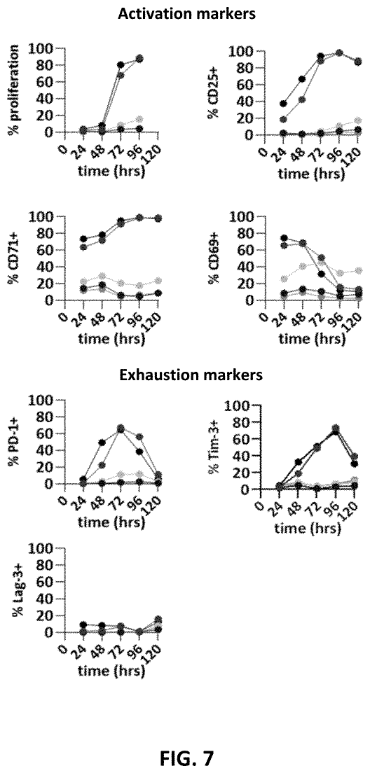

PBMCs were cocultured with C4-2B target cells as a 1:1 effector to target ratio (CD3 to target cells) for the indicated time points in the presence of the test multispecifics. At each time point, cells were harvested and CD3, CD4 and CD8 T cells were analyzed for the presence of the indicated activation and exhaustion markers. As shown in FIG. 7, results indicate low affinity CD3 multispecifics paired with CD8 binders specifically and potently activate only CD8 T cells.

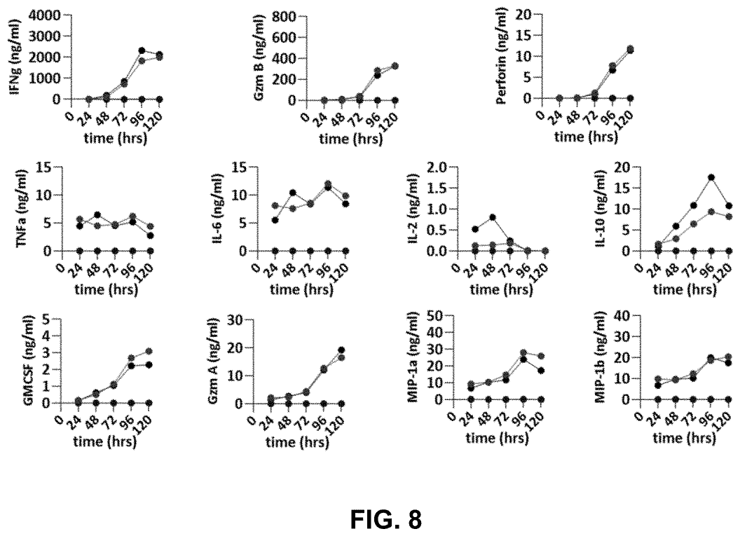

PBMCs were cocultured with C4-2B target cells as a 1:1 effector to target ratio (CD3 to target cells) for the indicated time points in the presence of the test multispecifics. At each time point, supernatants were harvested and analyzed for the indicated cytokines using a multiplex Luminex analysis system. The results indicate that low affinity CD3 multispecifics paired with CD8 binders show reduced anti-inflammatory cytokine release (see FIG. 8).

TABLE 14

| Antibody | combination | CD3 arm | Affinity |
|---|---|---|---|
| CD8B573.001 | CD3 × CD8 × PSMA | CD3B450 | Ultra low |
| CD8B574.001 | CD8 × PSMA | NA | |
| CD8B155.003 | CD3 × PSMA | CD3B450 | Ultra low |
| CD8B52 | PSMB410scFv × | CD3B376 | Medium |
| | CD3B376-Fab | | (40-60 nM) |
| VB19 | CD3B220 × PSMB365 | CD3B220 | high |

### Example 4: Production of Antibodies that Bind CD8

4.1: Generation CD8α Antibodies, CD8β Antibodies, and CD8αβ Antibodies

Immunogen. Recombinant human CD8alpha/beta heterodimer protein (cat #9358-CD) was obtained from R&D Systems, Inc. The amino acid sequence of the heterodimeric protein is listed in Table 15.

TABLE 15
Amino acid sequence of recombinant
human CD8α/β heterodimer protein

| | Protein | | SEQ |
|---|---|---|---|
| Name | ID | Sequence | ID NO |
| Recombinant | rhCD8α | SQFRVSPLDRTWNLGETVELK | 2177 |
| human | (Ser22- | CQVLLSNPTSGCSWLFQPRGA | 2322 |
| CD8α/β | Asp182) | AASPTFLLYLSQNKPKAAEGL | |
| heterodimer | Accession | DTQRFSGKRLGDTFVLTLSDF | |
| protein | #P01732 | RRENEGYYFCSALSNSIMYFS | |
| (cat #: | | HFVPVFLPAKPTTTPAPRPPT | |
| 9358-CD) | | PAPTIASQPLSLRPEACRPAA | |
| | | GGAVHTRGLDFACD- | |
| | | [proprietary R&D | |
| | | System acidic tails]- | |
| | | HHHHHH | |
| | rhCD8β | NSVLQQTPAYIKVQTNKMVML | 2178 |
| | (Asn19- | SCEAKISLSNMRIYWLRQRQA | 2323 |
| | Pro170) | PSSDSHHEFLALWDSAKGTIH | |
| | Accession | GEEVEQEKIAVFRDASRFILN | |
| | #P10966 | LTSVKPEDSGIYFCMIVGSPE | |
| | | LTFGKGTQLSVVDFLPTTAQP | |
| | | TKKSTLKKRVCRLPRPETQKG | |
| | | PLCSP-[proprietary | |
| | | R&D System basic | |
| | | tails]-DYKDDDDK | |

Immunization in wild-type mouse and screening of anti-CD8α antibodies, anti-CD8β antibodies, and anti-CD8αβ antibodies. Wild-type (WT) mice with 6 different MHC combinations was immunized using rapid immunization protocol. Eight mice were selected for cell fusion based on serum titer. Hybridoma supernatants were screening by LUMINEX using the immunogen and human pan-T cells. Hits were V-region recovered and formatted into monoclonal IgG1 antibodies.

All the monoclonal antibodies were produced as full-length antibodies as human IgG1. Nucleic acid sequences encoding variable regions were subcloned into a custom mammalian expression vectors containing constant region of IgG1 Fc expression cassettes using standard PCR restriction enzyme based cloning techniques. The mAbs were expressed by transient transfection in Chinese hamster ovary cell line. The antibodies were initially purified by MAB SELECT SURE Protein A column (GE healthcare, Piscataway, N.J.) (Brown, Bottomley et al. 1998). The column was equilibrated with Phosphate Buffer Saline (PBS), pH 7.2 and loaded with fermentation supernatant at a flow rate of 2 mL/min. After loading, the column was washed with PBS (4 CV) followed by elution in 30 mM sodium acetate, pH 3.5. Fractions containing protein peaks as monitored by Absorbance at 280 nm in AKTA Explorer (GE healthcare) were pooled together and were neutralized to pH 5.0 by adding 1% of 3 M sodium acetate, pH 9.0. As a polishing step, the antibodies were purified on a preparative size exclusion chromatography (SEC) using a SUPERDEX 200 column (GE healthcare). The integrity of the sample was assessed by endotoxin measurement and SDS polyacrylamide gel electrophoresis under reducing and non-reducing conditions. The intact mass was confirmed by mass spectrometry.

The VH and VL sequences of certain CD8 antibodies are provided in Table 16. The CDRs sequences of certain CD8 antibodies are provided in Table 17 (Kabat), Table 18 (Chothia), Table 19 (AbM), Table 20 (Contact), and Table 21 (IMGT).

TABLE 16
VH and VL Amino Acid Sequences

| | Protein | HC | LC | VH AA | VL AA | | Light Chain AA |
|---|---|---|---|---|---|---|---|
| # | Name | Isotype | Isotype | sequence | sequence | Heavy Chain AA sequence | sequence |
| 1 | CD8B191 | IgG1 | Kappa | QIQLVQSGPE | DIVLTQSP | QIQLVQSGPELVKPGTSMKMSCKASGYTFTDYYMNW | DIVLTQSPATLSVTPG |
| | | | | LVKPGTSMKM | ATLSVTPG | VKQSHGKSLEWIGRVIPSNGGTIYNLKFKGKATLTV | DRVSLSCRASQSISDF |
| | | | | SCKASGYTFT | DRVSLSCR | DKSLSTAYMQLNSLTSEDSAVYFCAREDYNNQGFFL | LHWYQQKSHESPRLLI |
| | | | | DYYMNWVKQS | ASQSISDF | DAMDYWGQGTSVTVSSASTKGPSVFPLAPSSKSTSG | KYASQSISGIPSRFSG |
| | | | | HGKSLEWIGR | LHWYQQKS | GTAALGCLVKDYFPEPVTVSWNSGALTSGVHTFPAV | SGSGSDFTLTINSVEP |
| | | | | VIPSNGGTIY | HESPRLLI | LQSSGLYSLSSVVTVPSSSLGTQTYICNVNHKPSNT | EDVGVYYCQNGHSFPY |
| | | | | NLKFKGKATL | KYASQSIS | KVDKKVEPKSCDKTHTCPPCPAPELLGGPSVFLFPP | TFGSGTKLEIKRTVAA |
| | | | | TVDKSLSTAY | GIPSRFSG | KPKDTLMISRTPEVTCVVVDVSHEDPEVKFNWYVDG | PSVFIFPPSDEQLKSG |
| | | | | MQLNSLTSED | SGSGSDFT | VEVHNAKTKPREEQYNSTYRVVSVLTVLHQDWLNGK | TASVVCLLNNFYPREA |
| | | | | SAVYFCARED | LTINSVEP | EYKCKVSNKALPAPIEKTISKAKGQPREPQVYTLPP | KVQWKVDNALQSGNSQ |
| | | | | YNNQGFFLDA | EDVGVYYC | SREEMTKNQVSLTCLVKGFYPSDIAVEWESNGQPEN | ESVTEQDSKDSTYSLS |
| | | | | MDYWGQGTSV | QNGHSFPY | NYKTTPPVLDSDGSFFLYSKLTVDKSRWQQGNVFSC | STLTLSKADYEKHKVY |
| | | | | TVSS | TFGSGTKL | SVMHEALHNHYTQKSLSLSPGK | ACEVTHQGLSSPVTKS |
| | | | | | EIK | | FNRGEC |
| | | | | 31 | 32 | 33 | 34 |
| 2 | CD8B226 | IgG1 | Kappa | EFQLQQSGPE | DIVMTQSP | EFQLQQSGPELVKPGASVKMSCKASGYTFTDYYMNW | DIVMTQSPATLSVTPG |
| | | | | LVKPGASVKM | ATLSVTPG | VKQSHGKSLQWIGRIIPSNGATIYNQKFKGKATLTV | DRVSLSCRASQSISHY |
| | | | | SCKASGYTFT | DRVSLSCR | DKSLSTAYMHLNSLTSEDSAVYYCAREDYSNQGFFL | LHWYQQKLHESPRLLI |
| | | | | DYYMNWVKQS | ASQSISHY | DAMDYWGQGTTVTVSSASTKGPSVFPLAPSSKSTSG | KYASQSISGIPSRFSG |
| | | | | HGKSLQWIGR | LHWYQQKL | GTAALGCLVKDYFPEPVTVSWNSGALTSGVHTFPAV | SGSGSDFTLSINSVEP |
| | | | | IIPSNGATIY | HESPRLLI | LQSSGLYSLSSVVTVPSSSLGTQTYICNVNHKPSNT | EDVGVYYCQNGHSFPY |
| | | | | NQKFKGKATL | KYASQSIS | KVDKKVEPKSCDKTHTCPPCPAPELLGGPSVFLFPP | TFGGGTKLEIKRTVAA |
| | | | | TVDKSLSTAY | GIPSRFSG | KPKDTLMISRTPEVTCVVVDVSHEDPEVKFNWYVDG | PSVFIFPPSDEQLKSG |
| | | | | MHLNSLTSED | SGSGSDFT | VEVHNAKTKPREEQYNSTYRVVSVLTVLHQDWLNGK | TASVVCLLNNFYPREA |
| | | | | SAVYYCARED | LSINSVEP | EYKCKVSNKALPAPIEKTISKAKGQPREPQVYTLPP | KVQWKVDNALQSGNSQ |
| | | | | YSNQGFFLDA | EDVGVYYC | SREEMTKNQVSLTCLVKGFYPSDIAVEWESNGQPEN | ESVTEQDSKDSTYSLS |
| | | | | MDYWGQGTTV | QNGHSFPY | NYKTTPPVLDSDGSFFLYSKLTVDKSRWQQGNVFSC | STLTLSKADYEKHKVY |
| | | | | TVSS | TFGGGTKL | SVMHEALHNHYTQKSLSLSPGK | ACEVTHQGLSSPVTKS |
| | | | | | EIK | | FNRGEC |
| | | | | 65 | 66 | 67 | 68 |
| 3 | CD8B259 | IgG1 | Kappa | EVQLQQSGPE | DIVMTQSP | EVQLQQSGPELVKPGASVKMSCKASGYTFTDYYMNW | DIVMTQSPATLSVTPG |
| | | | | LVKPGASVKM | ATLSVTPG | VKQSHGKSLEWIGRVIPSNGGTIYNQKFRGKATLTV | DRVSLSCRASQSISHF |
| | | | | SCKASGYTFT | DRVSLSCR | DKSLSTAYMQLNSLTSEDSAVYYCAREDYGNQGFFL | LHWYQQKSHESPRLLI |
| | | | | DYYMNWVKQS | ASQSISHF | DAMDYWGQGTTVTVSSASTKGPSVFPLAPSSKSTSG | KYASQSISGSPSKFSG |
| | | | | HGKSLEWIGR | LHWYQQKS | GTAALGCLVKDYFPEPVTVSWNSGALTSGVHTFPAV | SGSGSDFTLTINSVEP |
| | | | | VIPSNGGTIY | HESPRLLI | LQSSGLYSLSSVVTVPSSSLGTQTYICNVNHKPSNT | EDVGVYYCQSGHSFPY |
| | | | | NQKFRGKATL | KYASQSIS | KVDKKVEPKSCDKTHTCPPCPAPELLGGPSVFLFPP | TFGSGTKLEIKRTVAA |
| | | | | TVDKSLSTAY | GSPSKFSG | KPKDTLMISRTPEVTCVVVDVSHEDPEVKFNWYVDG | PSVFIFPPSDEQLKSG |
| | | | | MQLNSLTSED | SGSGSDFT | VEVHNAKTKPREEQYNSTYRVVSVLTVLHQDWLNGK | TASVVCLLNNFYPREA |
| | | | | SAVYYCARED | LTINSVEP | EYKCKVSNKALPAPIEKTISKAKGQPREPQVYTLPP | KVQWKVDNALQSGNSQ |
| | | | | YGNQGFFLDA | EDVGVYYC | SREEMTKNQVSLTCLVKGFYPSDIAVEWESNGQPEN | ESVTEQDSKDSTYSLS |
| | | | | MDYWGQGTTV | QSGHSFPY | NYKTTPPVLDSDGSFFLYSKLTVDKSRWQQGNVFSC | STLTLSKADYEKHKVY |
| | | | | TVSS | TFGSGTKL | SVMHEALHNHYTQKSLSLSPGK | ACEVTHQGLSSPVTKS |
| | | | | | EIK | | FNRGEC |
| | | | | 99 | 100 | 101 | 102 |
| 4 | CD8B298 | IgG1 | Kappa | QVQLQQSGPE | DIVMTQSP | QVQLQQSGPELVKPGASVKMSCKASGYTFTDYYMNW | DIVMTQSPATLSVTPG |
| | | | | LVKPGASVKM | ATLSVTPG | VKQSHGKSLEWIGRVIPNNGGTRYNQKFKGKATLTV | DRVSLSCRASQTISDY |
| | | | | SCKASGYTFT | DRVSLSCR | DKSLSTAYMQLNSLTSEDSAVYYCAREDFSNQGFFL | LHWYQQKSHESPRLLI |
| | | | | DYYMNWVKQS | ASQTISDY | DAMDYWGQGTSVTVSSASTKGPSVFPLAPSSKSTSG | KYASQSISGIPSRFSG |
| | | | | HGKSLEWIGR | LHWYQQKS | GTAALGCLVKDYFPEPVTVSWNSGALTSGVHTFPAV | SGSGSDFTLSINSVEP |
| | | | | VIPNNGGTRY | HESPRLLI | LQSSGLYSLSSVVTVPSSSLGTQTYICNVNHKPSNT | EDVGVYYCQNGHSFPY |
| | | | | NQKFKGKATL | KYASQSIS | KVDKKVEPKSCDKTHTCPPCPAPELLGGPSVFLFPP | TFGAGTKLELKRTVAA |
| | | | | TVDKSLSTAY | GIPSRFSG | KPKDTLMISRTPEVTCVVVDVSHEDPEVKFNWYVDG | PSVFIFPPSDEQLKSG |
| | | | | MQLNSLTSED | SGSGSDFT | VEVHNAKTKPREEQYNSTYRVVSVLTVLHQDWLNGK | TASVVCLLNNFYPREA |
| | | | | SAVYYCARED | LSINSVEP | EYKCKVSNKALPAPIEKTISKAKGQPREPQVYTLPP | KVQWKVDNALQSGNSQ |
| | | | | FSNQGFFLDA | EDVGVYYC | SREEMTKNQVSLTCLVKGFYPSDIAVEWESNGQPEN | ESVTEQDSKDSTYSLS |
| | | | | MDYWGQGTSV | QNGHSFPY | NYKTTPPVLDSDGSFFLYSKLTVDKSRWQQGNVFSC | STLTLSKADYEKHKVY |
| | | | | TVSS | TFGAGTKL | SVMHEALHNHYTQKSLSLSPGK | ACEVTHQGLSSPVTKS |
| | | | | | ELK | | FNRGEC |
| | | | | 133 | 134 | 135 | 136 |
| 5 | CD8B342 | IgG1 | Kappa | EFQLQQSGPE | DIVMTQTP | EFQLQQSGPELVKPGASVKVSCKASGYTFTDYYVNW | DIVMTQTPATLSVTPG |
| | | | | LVKPGASVKV | ATLSVTPG | VQQSHGKSLEWIGRVIPNNGNVIYNQNFKGKATLTV | DRVSLSCRASQTISNY |
| | | | | SCKASGYTFT | DRVSLSCR | DKSLSSAYLQLNSLTSEDSAVYYCTREDYSNQGFFL | LHWYQQKSHESPRLLI |
| | | | | DYYVNWVQQS | ASQTISNY | DAMDYWGQGTSVTVSSASTKGPSVFPLAPSSKSTSG | KYASQSISGIPSRFSG |
| | | | | HGKSLEWIGR | LHWYQQKS | GTAALGCLVKDYFPEPVTVSWNSGALTSGVHTFPAV | SGSGSDFTLSINSVEP |
| | | | | VIPNNGNVIY | HESPRLLI | LQSSGLYSLSSVVTVPSSSLGTQTYICNVNHKPSNT | EDVGVYYCQNGHSFPY |
| | | | | NQNFKGKATL | KYASQSIS | KVDKKVEPKSCDKTHTCPPCPAPELLGGPSVFLFPP | TFGGGTKLEIKRTVAA |
| | | | | TVDKSLSSAY | GIPSRFSG | KPKDTLMISRTPEVTCVVVDVSHEDPEVKFNWYVDG | PSVFIFPPSDEQLKSG |
| | | | | LQLNSLTSED | SGSGSDFT | VEVHNAKTKPREEQYNSTYRVVSVLTVLHQDWLNGK | TASVVCLLNNFYPREA |
| | | | | SAVYYCTRED | LSINSVEP | EYKCKVSNKALPAPIEKTISKAKGQPREPQVYTLPP | KVQWKVDNALQSGNSQ |
| | | | | YSNQGFFLDA | EDVGVYYC | SREEMTKNQVSLTCLVKGFYPSDIAVEWESNGQPEN | ESVTEQDSKDSTYSLS |
| | | | | MDYWGQGTSV | QNGHSFPY | NYKTTPPVLDSDGSFFLYSKLTVDKSRWQQGNVFSC | STLTLSKADYEKHKVY |
| | | | | TVSS | TFGGGTKL | SVMHEALHNHYTQKSLSLSPGK | ACEVTHQGLSSPVTKS |
| | | | | | EIK | | FNRGEC |
| | | | | 167 | 168 | 169 | 170 |
| 6 | CD8B364 | IgG1 | Kappa | QVQLQQPGAE | DIVLTQSP | QVQLQQPGAELVKPGASVKLSCKASGYTFTSYWMHW | DIVLTQSPASLSVATG |
| | | | | LVKPGASVKL | ASLSVATG | VNRRPGQGLEWIGEINPSNGDSYYNEKFKRKATLTV | EKVTIRCITSTDIDDD |
| | | | | SCKASGYTFT | EKVTIRCI | DISSSTAYMQLSSLTSEDSAVYYCTRSMYYDGRAGA | MNWYQQKPGEPPKLLI |
| | | | | SYWMHWVNRR | TSTDIDDD | YWGQGTTVTVSSASTKGPSVFPLAPSSKSTSGGTAA | SEGNTLRPGVPSRFSS |
| | | | | PGQGLEWIGE | MNWYQQKP | LGCLVKDYFPEPVTVSWNSGALTSGVHTFPAVLQSS | SGYGTDFVFTIENTLS |
| | | | | INPSNGDSYY | GEPPKLLI | GLYSLSSVVTVPSSSLGTQTYICNVNHKPSNTKVDK | EDVADYYCLQSDNMPL |
| | | | | NEKFKRKATL | SEGNTLRP | KVEPKSCDKTHTCPPCPAPELLGGPSVFLFPPKPKD | TFGAGTKLELKRTVAA |
| | | | | TVDISSSTAY | GVPSRFSS | TLMISRTPEVTCVVVDVSHEDPEVKFNWYVDGVEVH | PSVFIFPPSDEQLKSG |
| | | | | MQLSSLTSED | SGYGTDFV | NAKTKPREEQYNSTYRVVSVLTVLHQDWLNGKEYKC | TASVVCLLNNFYPREA |
| | | | | SAVYYCTRSM | FTIENTLS | KVSNKALPAPIEKTISKAKGQPREPQVYTLPPSREE | KVQWKVDNALQSGNSQ |
| | | | | YYDGRAGAYW | EDVADYYC | MTKNQVSLTCLVKGFYPSDIAVEWESNGQPENNYKT | ESVTEQDSKDSTYSLS |
| | | | | GQGTTVTVSS | LQSDNMPL | TPPVLDSDGSFFLYSKLTVDKSRWQQGNVFSCSVMH | STLTLSKADYEKHKVY |
| | | | | | TFGAGTKL | EALHNHYTQKSLSLSPGK | ACEVTHQGLSSPVTKS |
| | | | | | ELK | | FNRGEC |
| | | | | 201 | 202 | 203 | 204 |
| 7 | CD8B200 | IgG1 | Kappa | EVQLQQSGAE | DIQMTQTT | EVQLQQSGAELVKPGASVKLSCKASGYTFTNYWIHW | DIQMTQTTSSLSASLG |
| | | | | LVKPGASVKL | SSLSASLG | VKQRPGQGLEWIGNIDPSDSETHYNQKFKDKATLTV | DRVTITCRASQDISPY |
| | | | | SCKASGYTFT | DRVTITCR | DKSSSTAYMQLISLTSEDSAVYYCASGLTGTGYYWG | LNWYQQKPEGTIKLLI |
| | | | | NYWIHWVKQR | ASQDISPY | QGTTLTVSSASTKGPSVFPLAPSSKSTSGGTAALGC | YYTSKLHSGVPSRFSG |
| | | | | PGQGLEWIGN | LNWYQQKP | LVKDYFPEPVTVSWNSGALTSGVHTFPAVLQSSGLY | SGSGTDYSLTISNLEQ |
| | | | | IDPSDSETHY | EGTIKLLI | SLSSVVTVPSSSLGTQTYICNVNHKPSNTKVDKKVE | EDIATYFCQQDNTLPY |
| | | | | NQKFKDKATL | YYTSKLHS | PKSCDKTHTCPPCPAPELLGGPSVFLFPPKPKDTLM | TFGSGTKLELKRTVAA |
| | | | | TVDKSSSTAY | GVPSRFSG | ISRTPEVTCVVVDVSHEDPEVKFNWYVDGVEVHNAK | PSVFIFPPSDEQLKSG |
| | | | | MQLISLTSED | SGSGTDYS | TKPREEQYNSTYRVVSVLTVLHQDWLNGKEYKCKVS | TASVVCLLNNFYPREA |
| | | | | SAVYYCASGL | LTISNLEQ | NKALPAPIEKTISKAKGQPREPQVYTLPPSREEMTK | KVQWKVDNALQSGNSQ |
| | | | | TGTGYYWGQG | EDIATYFC | NQVSLTCLVKGFYPSDIAVEWESNGQPENNYKTTPP | ESVTEQDSKDSTYSLS |
| | | | | TTLTVSS | QQDNTLPY | VLDSDGSFFLYSKLTVDKSRWQQGNVFSCSVMHEAL | STLTLSKADYEKHKVY |
| | | | | | TFGSGTKL | HNHYTQKSLSLSPGK | ACEVTHQGLSSPVTKS |
| | | | | | ELK | | FNRGEC |
| | | | | 235 | 236 | 237 | 238 |
| 8 | CD8B247 | IgG1 | Kappa | EVQLQQSGPE | DIVMTQSP | EVQLQQSGPELVKPGASVKMSCKASGYTFTDYYMNW | DIVMTQSPATLSVTPG |
| | | | | LVKPGASVKM | ATLSVTPG | VKQSHGKSLEWIGRVIPNNGGTIYNQKFKDKATLTV | ERVSLSCRASQTISHF |
| | | | | SCKASGYTFT | ERVSLSCR | DKSLSTAYMQLNSLTSEDSAVYYCAREDYSNQGFFL | LHWYQQKSHESPRLLI |
| | | | | DYYMNWVKQS | ASQTISHF | DAMDYWGQGTSVTVSSASTKGPSVFPLAPSSKSTSG | KYASQSISGIPSRFSG |
| | | | | HGKSLEWIGR | LHWYQQKS | GTAALGCLVKDYFPEPVTVSWNSGALTSGVHTFPAV | GGSGSDFILTINSVEP |
| | | | | VIPNNGGTIY | HESPRLLI | LQSSGLYSLSSVVTVPSSSLGTQTYICNVNHKPSNT | EDVGMYYCQSGHSFPY |
| | | | | NQKFKDKATL | KYASQSIS | KVDKKVEPKSCDKTHTCPPCPAPELLGGPSVFLFPP | TFGSGTKLEIKRTVAA |
| | | | | TVDKSLSTAY | GIPSRFSG | KPKDTLMISRTPEVTCVVVDVSHEDPEVKFNWYVDG | PSVFIFPPSDEQLKSG |
| | | | | MQLNSLTSED | GGSGSDFI | VEVHNAKTKPREEQYNSTYRVVSVLTVLHQDWLNGK | TASVVCLLNNFYPREA |
| | | | | SAVYYCARED | LTINSVEP | EYKCKVSNKALPAPIEKTISKAKGQPREPQVYTLPP | KVQWKVDNALQSGNSQ |
| | | | | YSNQGFFLDA | EDVGMYYC | SREEMTKNQVSLTCLVKGFYPSDIAVEWESNGQPEN | ESVTEQDSKDSTYSLS |
| | | | | MDYWGQGTSV | QSGHSFPY | NYKTTPPVLDSDGSFFLYSKLTVDKSRWQQGNVFSC | STLTLSKADYEKHKVY |
| | | | | TVSS | TFGSGTKL | SVMHEALHNHYTQKSLSLSPGK | ACEVTHQGLSSPVTKS |
| | | | | | EIK | | FNRGEC |
| | | | | 269 | 270 | 271 | 272 |
| 9 | CD8B265 | IgG1 | Kappa | QVQLQQSGPE | DIVMTQSP | QVQLQQSGPELVKPGASVKMSCKASGYSFTDYYMNW | DIVMTQSPATLSVTPG |
| | | | | LVKPGASVKM | ATLSVTPG | VKQSHGQSLEWIGRVIPRNGATTYNQNFRGKATLTV | DRVSLSCRASQSISHY |
| | | | | SCKASGYSFT | DRVSLSCR | DISLRTAYMHLNSLTSDDSAVYYCAREDFSNQGFFL | LHWYQQKSHESPRLLI |
| | | | | DYYMNWVKQS | ASQSISHY | DAMDYWGQGTSVTVSSASTKGPSVFPLAPSSKSTSG | KYASQSISGIPSRFSG |
| | | | | HGQSLEWIGR | LHWYQQKS | GTAALGCLVKDYFPEPVTVSWNSGALTSGVHTFPAV | SGSGSDFTLSINSVEP |
| | | | | VIPRNGATTY | HESPRLLI | LQSSGLYSLSSVVTVPSSSLGTQTYICNVNHKPSNT | EDVGVYYCQNGHSFPY |
| | | | | NQNFRGKATL | KYASQSIS | KVDKKVEPKSCDKTHTCPPCPAPELLGGPSVFLFPP | TFGSGTKLEMKRTVAA |
| | | | | TVDISLRTAY | GIPSRFSG | KPKDTLMISRTPEVTCVVVDVSHEDPEVKFNWYVDG | PSVFIFPPSDEQLKSG |
| | | | | MHLNSLTSDD | SGSGSDFT | VEVHNAKTKPREEQYNSTYRVVSVLTVLHQDWLNGK | TASVVCLLNNFYPREA |
| | | | | SAVYYCARED | LSINSVEP | EYKCKVSNKALPAPIEKTISKAKGQPREPQVYTLPP | KVQWKVDNALQSGNSQ |
| | | | | FSNQGFFLDA | EDVGVYYC | SREEMTKNQVSLTCLVKGFYPSDIAVEWESNGQPEN | ESVTEQDSKDSTYSLS |
| | | | | MDYWGQGTSV | QNGHSFPY | NYKTTPPVLDSDGSFFLYSKLTVDKSRWQQGNVFSC | STLTLSKADYEKHKVY |
| | | | | TVSS | TFGSGTKL | SVMHEALHNHYTQKSLSLSPGK | ACEVTHQGLSSPVTKS |
| | | | | | EMK | | FNRGEC |
| | | | | 303 | 304 | 305 | 306 |
| 10 | CD8B270 | IgG1 | Kappa | QVQLQQPGAE | DIQMTQTT | QVQLQQPGAELVKPGASVMLSCKASGYTFTNYWMHW | DIQMTQTTSSLSASLG |
| | | | | LVKPGASVML | SSLSASLG | VKQRPGQGLEWIGNIDPSDSETHYNQKFKDKATLTV | DRVTITCRASQDIRPY |
| | | | | SCKASGYTFT | DRVTITCR | DKSSSTAYMQLSSLTSEDSAVYYCASGLTGTGYYWG | LNWYQQKPEGTIKLLI |
| | | | | NYWMHWVKQR | ASQDIRPY | QGTTLTVSSASTKGPSVFPLAPSSKSTSGGTAALGC | YFTSKLHSGVPSRFSG |
| | | | | PGQGLEWIGN | LNWYQQKP | LVKDYFPEPVTVSWNSGALTSGVHTFPAVLQSSGLY | SGSGTDYSLTISNLEQ |
| | | | | IDPSDSETHY | EGTIKLLI | SLSSVVTVPSSSLGTQTYICNVNHKPSNTKVDKKVE | EDIATYFCQQDNTLPY |
| | | | | NQKFKDKATL | YFTSKLHS | PKSCDKTHTCPPCPAPELLGGPSVFLFPPKPKDTLM | TFGSGTKLELKRTVAA |
| | | | | TVDKSSSTAY | GVPSRFSG | ISRTPEVTCVVVDVSHEDPEVKFNWYVDGVEVHNAK | PSVFIFPPSDEQLKSG |
| | | | | MQLSSLTSED | SGSGTDYS | TKPREEQYNSTYRVVSVLTVLHQDWLNGKEYKCKVS | TASVVCLLNNFYPREA |
| | | | | SAVYYCASGL | LTISNLEQ | NKALPAPIEKTISKAKGQPREPQVYTLPPSREEMTK | KVQWKVDNALQSGNSQ |
| | | | | TGTGYYWGQG | EDIATYFC | NQVSLTCLVKGFYPSDIAVEWESNGQPENNYKTTPP | ESVTEQDSKDSTYSLS |
| | | | | TTLTVSS | QQDNTLPY | VLDSDGSFFLYSKLTVDKSRWQQGNVFSCSVMHEAL | STLTLSKADYEKHKVY |
| | | | | | TFGSGTKL | HNHYTQKSLSLSPGK | ACEVTHQGLSSPVTKS |
| | | | | | ELK | | FNRGEC |
| | | | | 337 | 338 | 339 | 340 |
| 11 | CD8B213 | IgG1 | Kappa | EVQLQQSGPE | DIVLTQSQ | EVQLQQSGPELVKPGDSMKMSCKASGYIFTDYYMDW | DIVLTQSQKFMSTSVG |
| | | | | LVKPGDSMKM | KFMSTSVG | VKQSHGKSLEWIGYIYPNNGITSYNQKFKGRATLTI | DRVSVTCKASQNVDKY |
| | | | | SCKASGYIFT | DRVSVTCK | DKSSSTAYMELHSLTSEDSAVYYCARSIYYDHGGGF | VAWYQQKPGQSPKALI |
| | | | | DYYMDWVKQS | ASQNVDKY | PYWGQGTSVTVSSASTKGPSVFPLAPSSKSTSGGTA | YSASYRYSGVPDRFTG |
| | | | | HGKSLEWIGY | VAWYQQKP | ALGCLVKDYFPEPVTVSWNSGALTSGVHTFPAVLQS | SGSGTDFTLTISNVQS |
| | | | | IYPNNGITSY | GQSPKALI | SGLYSLSSVVTVPSSSLGTQTYICNVNHKPSNTKVD | EDLAEYFCQQYNTYPS |
| | | | | NQKFKGRATL | YSASYRYS | KKVEPKSCDKTHTCPPCPAPELLGGPSVFLFPPKPK | FGSGTKLEMKRTVAAP |
| | | | | TIDKSSSTAY | GVPDRFTG | DTLMISRTPEVTCVVVDVSHEDPEVKFNWYVDGVEV | SVFIFPPSDEQLKSGT |
| | | | | MELHSLTSED | SGSGTDFT | HNAKTKPREEQYNSTYRVVSVLTVLHQDWLNGKEYK | ASVVCLLNNFYPREAK |
| | | | | SAVYYCARSI | LTISNVQS | CKVSNKALPAPIEKTISKAKGQPREPQVYTLPPSRE | VQWKVDNALQSGNSQE |
| | | | | YYDHGGGFPY | EDLAEYFC | EMTKNQVSLTCLVKGFYPSDIAVEWESNGQPENNYK | SVTEQDSKDSTYSLSS |
| | | | | WGQGTSVTVS | QQYNTYPS | TTPPVLDSDGSFFLYSKLTVDKSRWQQGNVFSCSVM | TLTLSKADYEKHKVYA |
| | | | | S | FGSGTKLE | HEALHNHYTQKSLSLSPGK | CEVTHQGLSSPVTKSF |
| | | | | | MK | | NRGEC |
| | | | | 371 | 372 | 373 | 374 |
| 12 | CD8B240 | IgG1 | Kappa | QVQLQQSGPE | DIVMTQSP | QVQLQQSGPELVKPGTSVKMSCKASGYTFTDYYMNW | DIVMTQSPATLSVTPG |
| | | | | LVKPGTSVKM | ATLSVTPG | VKQSHGKSLEWIGRVIPSNGGTIYNLKFKGKATLTV | DRVSLSCRASQSISDF |
| | | | | SCKASGYTFT | DRVSLSCR | DKSLSTAYMQLNSLTSEDSAVYFCAREDYNNQGFFL | LHWYQQKSHESPRLLI |
| | | | | DYYMNWVKQS | ASQSISDF | DAMDYWGQGTLVTVSAASTKGPSVFPLAPSSKSTSG | KYASQSISGIPSRFSG |
| | | | | HGKSLEWIGR | LHWYQQKS | GTAALGCLVKDYFPEPVTVSWNSGALTSGVHTFPAV | SGSGSDFTLTINSVEP |
| | | | | VIPSNGGTIY | HESPRLLI | LQSSGLYSLSSVVTVPSSSLGTQTYICNVNHKPSNT | EDVGVYYCQNGHSFPY |
| | | | | NLKFKGKATL | KYASQSIS | KVDKKVEPKSCDKTHTCPPCPAPELLGGPSVFLFPP | TFGSGTKLEIKRTVAA |
| | | | | TVDKSLSTAY | GIPSRFSG | KPKDTLMISRTPEVTCVVVDVSHEDPEVKFNWYVDG | PSVFIFPPSDEQLKSG |
| | | | | MQLNSLTSED | SGSGSDFT | VEVHNAKTKPREEQYNSTYRVVSVLTVLHQDWLNGK | TASVVCLLNNFYPREA |
| | | | | SAVYFCARED | LTINSVEP | EYKCKVSNKALPAPIEKTISKAKGQPREPQVYTLPP | KVQWKVDNALQSGNSQ |
| | | | | YNNQGFFLDA | EDVGVYYC | SREEMTKNQVSLTCLVKGFYPSDIAVEWESNGQPEN | ESVTEQDSKDSTYSLS |
| | | | | MDYWGQGTLV | QNGHSFPY | NYKTTPPVLDSDGSFFLYSKLTVDKSRWQQGNVFSC | STLTLSKADYEKHKVY |
| | | | | TVSA | TFGSGTKL | SVMHEALHNHYTQKSLSLSPGK | ACEVTHQGLSSPVTKS |
| | | | | | EIK | | FNRGEC |
| | | | | 405 | 406 | 407 | 408 |
| 13 | CD8B361 | IgG1 | Kappa | EVQLQQSGPE | DIVMTQSQ | EVQLQQSGPELVKPGNSVKMSCKASGYTFTDYYMDW | DIVMTQSQKFMSTSVG |
| | | | | LVKPGNSVKM | KFMSTSVG | VKQSHGTSLEWIGYIYPNNGDTRYNQKFKDKATLTV | DRVSVTCKASQNVGTY |
| | | | | SCKASGYTFT | DRVSVTCK | DKSSSTAYMELHSLTSEDSAVFYCARSIYYDHGGGF | VAWYQQKPGQSPKALI |
| | | | | DYYMDWVKQS | ASQNVGTY | PYWGQGTLVTVSAASTKGPSVFPLAPSSKSTSGGTA | YSASYRYSGVPDRFTG |
| | | | | HGTSLEWIGY | VAWYQQKP | ALGCLVKDYFPEPVTVSWNSGALTSGVHTFPAVLQS | SGSGTDFTLTINNVQS |
| | | | | IYPNNGDTRY | GQSPKALI | SGLYSLSSVVTVPSSSLGTQTYICNVNHKPSNTKVD | EDLAEYLCQQYNSYPT |
| | | | | NQKFKDKATL | YSASYRYS | KKVEPKSCDKTHTCPPCPAPELLGGPSVFLFPPKPK | FGGGTRLEIKRTVAAP |
| | | | | TVDKSSSTAY | GVPDRFTG | DTLMISRTPEVTCVVVDVSHEDPEVKFNWYVDGVEV | SVFIFPPSDEQLKSGT |
| | | | | MELHSLTSED | SGSGTDFT | HNAKTKPREEQYNSTYRVVSVLTVLHQDWLNGKEYK | ASVVCLLNNFYPREAK |
| | | | | SAVFYCARSI | LTINNVQS | CKVSNKALPAPIEKTISKAKGQPREPQVYTLPPSRE | VQWKVDNALQSGNSQE |
| | | | | YYDHGGGFPY | EDLAEYLC | EMTKNQVSLTCLVKGFYPSDIAVEWESNGQPENNYK | SVTEQDSKDSTYSLSS |
| | | | | WGQGTLVTVS | QQYNSYPT | TTPPVLDSDGSFFLYSKLTVDKSRWQQGNVFSCSVM | TLTLSKADYEKHKVYA |
| | | | | A | FGGGTRLE | HEALHNHYTQKSLSLSPGK | CEVTHQGLSSPVTKSF |
| | | | | | IK | | NRGEC |
| | | | | 439 | 440 | 441 | 442 |
| 14 | CD8B246 | IgG1 | Kappa | QVQLKESGPG | DIQMTQTT | QVQLKESGPGILKPSQTLSLTCSFSGFSLSTSGMNV | DIQMTQTTSSLSASLG |
| | | | | ILKPSQTLSL | SSLSASLG | GWIRQPSGKGLEWLAHIWWDDDKYYNPSLKSQLTIS | DRVTISCRASQDIRNY |
| | | | | TCSFSGFSLS | DRVTISCR | KDTSRNQVFLKITSVDTADTATYYCARRGNYGNYEF | LNWYQQKPDGTVKLLI |
| | | | | TSGMNVGWIR | ASQDIRNY | AYWGQGTTLTVSSASTKGPSVFPLAPSSKSTSGGTA | YHTSRLHSGVPSRFSG |
| | | | | QPSGKGLEWL | LNWYQQKP | ALGCLVKDYFPEPVTVSWNSGALTSGVHTFPAVLQS | SGSGTDYSLTISNLEQ |
| | | | | AHIWWDDDKY | DGTVKLLI | SGLYSLSSVVTVPSSSLGTQTYICNVNHKPSNTKVD | EDIATYFCQQGNTLPW |
| | | | | YNPSLKSQLT | YHTSRLHS | KKVEPKSCDKTHTCPPCPAPELLGGPSVFLFPPKPK | TFGAGTKLELKRTVAA |
| | | | | ISKDTSRNQV | GVPSRFSG | DTLMISRTPEVTCVVVDVSHEDPEVKFNWYVDGVEV | PSVFIFPPSDEQLKSG |
| | | | | FLKITSVDTA | SGSGTDYS | HNAKTKPREEQYNSTYRVVSVLTVLHQDWLNGKEYK | TASVVCLLNNFYPREA |
| | | | | DTATYYCARR | LTISNLEQ | CKVSNKALPAPIEKTISKAKGQPREPQVYTLPPSRE | KVQWKVDNALQSGNSQ |
| | | | | GNYGNYEFAY | EDIATYFC | EMTKNQVSLTCLVKGFYPSDIAVEWESNGQPENNYK | ESVTEQDSKDSTYSLS |
| | | | | WGQGTTLTVS | QQGNTLPW | TTPPVLDSDGSFFLYSKLTVDKSRWQQGNVFSCSVM | STLTLSKADYEKHKVY |
| | | | | S | TFGAGTKL | HEALHNHYTQKSLSLSPGK | ACEVTHQGLSSPVTKS |
| | | | | | ELK | | FNRGEC |
| | | | | 473 | 474 | 475 | 476 |
| 15 | CD8B268 | IgG1 | Kappa | QVQLQQSGAE | DIQMTQSP | QVQLQQSGAELVKPGASVKLSCKASGYTFTVYTIHW | DIQMTQSPASLSASVG |
| | | | | LVKPGASVKL | ASLSASVG | VKQRSGQGLEWIGWFYPGSGNIKYNEKFKDKATLTA | QTVTITCRASGNIHNY |
| | | | | SCKASGYTFT | QTVTITCR | DKSSHTVYMELSRLTSEDSAVYFCARHEDNHYYDGN | LAWFQQKQGKSPQLLV |
| | | | | VYTIHWVKQR | ASGNIHNY | SWFAYWGQGTLVTVSAASTKGPSVFPLAPSSKSTSG | YNAKTLADGVPSRFSG |
| | | | | SGQGLEWIGW | LAWFQQKQ | GTAALGCLVKDYFPEPVTVSWNSGALTSGVHTFPAV | SGSGTQYSLKINSLQT |
| | | | | FYPGSGNIKY | GKSPQLLV | LQSSGLYSLSSVVTVPSSSLGTQTYICNVNHKPSNT | EDFGNYYCQHFWNTPY |
| | | | | NEKFKDKATL | YNAKTLAD | KVDKKVEPKSCDKTHTCPPCPAPELLGGPSVFLFPP | TFGGGTKLEIKRTVAA |
| | | | | TADKSSHTVY | GVPSRFSG | KPKDTLMISRTPEVTCVVVDVSHEDPEVKFNWYVDG | PSVFIFPPSDEQLKSG |
| | | | | MELSRLTSED | SGSGTQYS | VEVHNAKTKPREEQYNSTYRVVSVLTVLHQDWLNGK | TASVVCLLNNFYPREA |
| | | | | SAVYFCARHE | LKINSLQT | EYKCKVSNKALPAPIEKTISKAKGQPREPQVYTLPP | KVQWKVDNALQSGNSQ |
| | | | | DNHYYDGNSW | EDFGNYYC | SREEMTKNQVSLTCLVKGFYPSDIAVEWESNGQPEN | ESVTEQDSKDSTYSLS |
| | | | | FAYWGQGTLV | QHFWNTPY | NYKTTPPVLDSDGSFFLYSKLTVDKSRWQQGNVFSC | STLTLSKADYEKHKVY |
| | | | | TVSA | TFGGGTKL | SVMHEALHNHYTQKSLSLSPGK | ACEVTHQGLSSPVTKS |
| | | | | | EIK | | FNRGEC |
| | | | | 507 | 508 | 509 | 510 |
| 16 | CD8B271 | IgG1 | Kappa | DVQLQESGPG | DIQMTQTT | DVQLQESGPGLVAPSQSLSITCTVSGFSLSIYSIHW | DIQMTQTTSSLSASLG |
| | | | | LVAPSQSLSI | SSLSASLG | VRQPPGKGLEWLGMIWGGGDTDYNSALKSRLSISKD | DRVTISCSASQGISNY |
| | | | | TCTVSGFSLS | DRVTISCS | NSESQVFLKMNSLQTDDTAMYYCARNPHYYGGTYEY | LNWYQQKPDGTVKLLI |
| | | | | IYSIHWVRQP | ASQGISNY | FDVWGTGTTVTVSSASTKGPSVFPLAPSSKSTSGGT | YDTSILYSGVPSRFSG |
| | | | | PGKGLEWLGM | LNWYQQKP | AALGCLVKDYFPEPVTVSWNSGALTSGVHTFPAVLQ | SGSGTDYSLTISNLEP |
| | | | | IWGGGDTDYN | DGTVKLLI | SSGLYSLSSVVTVPSSSLGTQTYICNVNHKPSNTKV | EDVATYYCQQYSNLPY |
| | | | | SALKSRLSIS | YDTSILYS | DKKVEPKSCDKTHTCPPCPAPELLGGPSVFLFPPKP | TFGSGTKLEIKRTVAA |
| | | | | KDNSESQVFL | GVPSRFSG | KDTLMISRTPEVTCVVVDVSHEDPEVKFNWYVDGVE | PSVFIFPPSDEQLKSG |
| | | | | KMNSLQTDDT | SGSGTDYS | VHNAKTKPREEQYNSTYRVVSVLTVLHQDWLNGKEY | TASVVCLLNNFYPREA |
| | | | | AMYYCARNPH | LTISNLEP | KCKVSNKALPAPIEKTISKAKGQPREPQVYTLPPSR | KVQWKVDNALQSGNSQ |
| | | | | YYGGTYEYFD | EDVATYYC | EEMTKNQVSLTCLVKGFYPSDIAVEWESNGQPENNY | ESVTEQDSKDSTYSLS |
| | | | | VWGTGTTVTV | QQYSNLPY | KTTPPVLDSDGSFFLYSKLTVDKSRWQQGNVFSCSV | STLTLSKADYEKHKVY |
| | | | | SS | TFGSGTKL | MHEALHNHYTQKSLSLSPGK | ACEVTHQGLSSPVTKS |
| | | | | | EIK | | FNRGEC |
| | | | | 541 | 542 | 543 | 544 |
| 17 | CD8B273 | IgG1 | Kappa | QVQLQQSGAE | DIQMTQSP | QVQLQQSGAELVKPGASVKLSCKASGYTFTEYTIHW | DIQMTQSPASLSASVG |
| | | | | LVKPGASVKL | ASLSASVG | VKQRSGQGLEWIGWFYPGTGSIKYNEKFKDKATLTA | ETVTITCRASGNIHNY |
| | | | | SCKASGYTFT | ETVTITCR | DKSSHTVYMELSKLTSEDSAVYFCARHEDNHYYDGN | LAWFQQKQGKSPQLLV |
| | | | | EYTIHWVKQR | ASGNIHNY | SWFAYWGQGTLVTVSAASTKGPSVFPLAPSSKSTSG | YNAKTLADGVPSRFSG |
| | | | | SGQGLEWIGW | LAWFQQKQ | GTAALGCLVKDYFPEPVTVSWNSGALTSGVHTFPAV | SGSGTQYSLKINSLQA |
| | | | | FYPGTGSIKY | GKSPQLLV | LQSSGLYSLSSVVTVPSSSLGTQTYICNVNHKPSNT | EDFGSYYCQHFWSTPY |
| | | | | NEKFKDKATL | YNAKTLAD | KVDKKVEPKSCDKTHTCPPCPAPELLGGPSVFLFPP | TFGSGTKLEIKRTVAA |
| | | | | TADKSSHTVY | GVPSRFSG | KPKDTLMISRTPEVTCVVVDVSHEDPEVKFNWYVDG | PSVFIFPPSDEQLKSG |
| | | | | MELSKLTSED | SGSGTQYS | VEVHNAKTKPREEQYNSTYRVVSVLTVLHQDWLNGK | TASVVCLLNNFYPREA |
| | | | | SAVYFCARHE | LKINSLQA | EYKCKVSNKALPAPIEKTISKAKGQPREPQVYTLPP | KVQWKVDNALQSGNSQ |
| | | | | DNHYYDGNSW | EDFGSYYC | SREEMTKNQVSLTCLVKGFYPSDIAVEWESNGQPEN | ESVTEQDSKDSTYSLS |
| | | | | FAYWGQGTLV | QHFWSTPY | NYKTTPPVLDSDGSFFLYSKLTVDKSRWQQGNVFSC | STLTLSKADYEKHKVY |
| | | | | TVSA | TFGSGTKL | SVMHEALHNHYTQKSLSLSPGK | ACEVTHQGLSSPVTKS |
| | | | | | EIK | | FNRGEC |
| | | | | 575 | 576 | 577 | 578 |
| 18 | CD8B288 | IgG1 | Kappa | QVQLQQSGAE | DIQMTQSP | QVQLQQSGAELVKPGASVKLSCKASGYTFTEYTIHW | DIQMTQSPASLSASVG |
| | | | | LVKPGASVKL | ASLSASVG | VKQKSGQGLEWIGWFYPGNGNMRYNEKFKDKATLTA | DTVTITCRASGNIHNY |
| | | | | SCKASGYTFT | DTVTITCR | DRSSHTVYMELSRLTSEDSAVYFCARYEDNHYYDGA | LAWFQQKQGKSPQLLV |
| | | | | EYTIHWVKQK | ASGNIHNY | SWFAYWGQGTSVTVSSASTKGPSVFPLAPSSKSTSG | YNAKTLADGVPSRFSG |
| | | | | SGQGLEWIGW | LAWFQQKQ | GTAALGCLVKDYFPEPVTVSWNSGALTSGVHTFPAV | SGSGTQFSLKINSLQP |
| | | | | FYPGNGNMRY | GKSPQLLV | LQSSGLYSLSSVVTVPSSSLGTQTYICNVNHKPSNT | EDFGTYYCQHFWSTPF |
| | | | | NEKFKDKATL | YNAKTLAD | KVDKKVEPKSCDKTHTCPPCPAPELLGGPSVFLFPP | TFGSGTKLEMKRTVAA |
| | | | | TADRSSHTVY | GVPSRFSG | KPKDTLMISRTPEVTCVVVDVSHEDPEVKFNWYVDG | PSVFIFPPSDEQLKSG |
| | | | | MELSRLTSED | SGSGTQFS | VEVHNAKTKPREEQYNSTYRVVSVLTVLHQDWLNGK | TASVVCLLNNFYPREA |
| | | | | SAVYFCARYE | LKINSLQP | EYKCKVSNKALPAPIEKTISKAKGQPREPQVYTLPP | KVQWKVDNALQSGNSQ |
| | | | | DNHYYDGASW | EDFGTYYC | SREEMTKNQVSLTCLVKGFYPSDIAVEWESNGQPEN | ESVTEQDSKDSTYSLS |
| | | | | FAYWGQGTSV | QHFWSTPF | NYKTTPPVLDSDGSFFLYSKLTVDKSRWQQGNVFSC | STLTLSKADYEKHKVY |
| | | | | TVSS | TFGSGTKL | SVMHEALHNHYTQKSLSLSPGK | ACEVTHQGLSSPVTKS |
| | | | | | EMK | | FNRGEC |
| | | | | 609 | 610 | 611 | 612 |
| 19 | CD8B292 | IgG1 | Kappa | QVQLQQPGAE | QIVLTQSP | QVQLQQPGAELVKPGASVKLSCTGSGFNFKDDYIYW | QIVLTQSPAIMSASLG |
| | | | | LVKPGASVKL | AIMSASLG | VKQRPEQGLEWIGWIDPENGATEYASKFQGKATITA | ERVTLTCTASSSVSSS |
| | | | | SCTGSGFNFK | ERVTLTCT | DTSSNIAYLQLSSLTSEDTAVYYCSLHDYGYAMDYW | YLHWYQQKPGSSPKLW |
| | | | | DDYIYWVKQR | ASSSVSSS | GQGTSVTVSSASTKGPSVFPLAPSSKSTSGGTAALG | IYSTSNLASGVPARFS |
| | | | | PEQGLEWIGW | YLHWYQQK | CLVKDYFPEPVTVSWNSGALTSGVHTFPAVLQSSGL | GSGSGTSYSLTISNME |
| | | | | IDPENGATEY | PGSSPKLW | YSLSSVVTVPSSSLGTQTYICNVNHKPSNTKVDKKV | AEDAATYYCHQYHRSP |
| | | | | ASKFQGKATI | IYSTSNLA | EPKSCDKTHTCPPCPAPELLGGPSVFLFPPKPKDTL | LTFGGGTKLEIKRTVA |
| | | | | TADTSSNIAY | SGVPARFS | MISRTPEVTCVVVDVSHEDPEVKFNWYVDGVEVHNA | APSVFIFPPSDEQLKS |
| | | | | LQLSSLTSED | GSGSGTSY | KTKPREEQYNSTYRVVSVLTVLHQDWLNGKEYKCKV | GTASVVCLLNNFYPRE |
| | | | | TAVYYCSLHD | SLTISNME | SNKALPAPIEKTISKAKGQPREPQVYTLPPSREEMT | AKVQWKVDNALQSGNS |
| | | | | YGYAMDYWGQ | AEDAATYY | KNQVSLTCLVKGFYPSDIAVEWESNGQPENNYKTTP | QESVTEQDSKDSTYSL |
| | | | | GTSVTVSS | CHQYHRSP | PVLDSDGSFFLYSKLTVDKSRWQQGNVFSCSVMHEA | SSTLTLSKADYEKHKV |
| | | | | | LTFGGGTK | LHNHYTQKSLSLSPGK | YACEVTHQGLSSPVTK |
| | | | | | LEIK | | SFNRGEC |
| | | | | 643 | 644 | 645 | 646 |
| 20 | CD8B303 | IgG1 | Kappa | QVQLKESGPG | DVQMIQSP | QVQLKESGPGLVAPSQSLSITCTVSGFSLSIYSIHW | DVQMIQSPSSLSASLG |
| | | | | LVAPSQSLSI | SSLSASLG | VRQPPGKGLEWLGMIWGGGSTDYNSTLNSRLSIIKD | GTVTITCKASQDIKKY |
| | | | | TCTVSGFSLS | GTVTITCK | NSKSQVFLKMNSLQTDDTAMYYCARNPHHYGGSTGA | MAWYQHKPGKGPRLLI |
| | | | | IYSIHWVRQP | ASQDIKKY | MDYWGQGTTVTVSSASTKGPSVFPLAPSSKSTSGGT | HYTSSLQPGIPSRFSG |
| | | | | PGKGLEWLGM | MAWYQHKP | AALGCLVKDYFPEPVTVSWNSGALTSGVHTFPAVLQ | SGSGRDYYFSISNLEP |
| | | | | IWGGGSTDYN | GKGPRLLI | SSGLYSLSSVVTVPSSSLGTQTYICNVNHKPSNTKV | EDIATYFCLQYDNLFT |
| | | | | STLNSRLSII | HYTSSLQP | DKKVEPKSCDKTHTCPPCPAPELLGGPSVFLFPPKP | FGSGTKLELKRTVAAP |
| | | | | KDNSKSQVFL | GIPSRFSG | KDTLMISRTPEVTCVVVDVSHEDPEVKFNWYVDGVE | SVFIFPPSDEQLKSGT |
| | | | | KMNSLQTDDT | SGSGRDYY | VHNAKTKPREEQYNSTYRVVSVLTVLHQDWLNGKEY | ASVVCLLNNFYPREAK |
| | | | | AMYYCARNPH | FSISNLEP | KCKVSNKALPAPIEKTISKAKGQPREPQVYTLPPSR | VQWKVDNALQSGNSQE |
| | | | | HYGGSTGAMD | EDIATYFC | EEMTKNQVSLTCLVKGFYPSDIAVEWESNGQPENNY | SVTEQDSKDSTYSLSS |
| | | | | YWGQGTTVTV | LQYDNLFT | KTTPPVLDSDGSFFLYSKLTVDKSRWQQGNVFSCSV | TLTLSKADYEKHKVYA |
| | | | | SS | FGSGTKLE | MHEALHNHYTQKSLSLSPGK | CEVTHQGLSSPVTKSF |
| | | | | | LK | | NRGEC |
| | | | | 677 | 678 | 679 | 680 |
| 21 | CD8B304 | IgG1 | Kappa | QVTLKESGPG | DIQMTQTT | QVTLKESGPGILKPSQTLSLTCSFSGFSLSTSGMNV | DIQMTQTTSSLSASLG |
| | | | | ILKPSQTLSL | SSLSASLG | GWIRQPSGKGLEWLAHIWWDDDKYYNPSLKSQLTIS | DRVTISCRASQDIRNY |
| | | | | TCSFSGFSLS | DRVTISCR | KDTSRNQVFLKITSVDTADTATYYCARRGNYGNYEF | LNWYQQKPDGTVKLLI |
| | | | | TSGMNVGWIR | ASQDIRNY | AYWGQGTTVTVSSASTKGPSVFPLAPSSKSTSGGTA | YHTSRLHSGVPSRFSG |
| | | | | QPSGKGLEWL | LNWYQQKP | ALGCLVKDYFPEPVTVSWNSGALTSGVHTFPAVLQS | SGSGTDYSLTISNLDQ |
| | | | | AHIWWDDDKY | DGTVKLLI | SGLYSLSSVVTVPSSSLGTQTYICNVNHKPSNTKVD | EDIATYFCQQGNTLPW |
| | | | | YNPSLKSQLT | YHTSRLHS | KKVEPKSCDKTHTCPPCPAPELLGGPSVFLFPPKPK | TFGAGTKLELKRTVAA |
| | | | | ISKDTSRNQV | GVPSRFSG | DTLMISRTPEVTCVVVDVSHEDPEVKFNWYVDGVEV | PSVFIFPPSDEQLKSG |
| | | | | FLKITSVDTA | SGSGTDYS | HNAKTKPREEQYNSTYRVVSVLTVLHQDWLNGKEYK | TASVVCLLNNFYPREA |
| | | | | DTATYYCARR | LTISNLDQ | CKVSNKALPAPIEKTISKAKGQPREPQVYTLPPSRE | KVQWKVDNALQSGNSQ |
| | | | | GNYGNYEFAY | EDIATYFC | EMTKNQVSLTCLVKGFYPSDIAVEWESNGQPENNYK | ESVTEQDSKDSTYSLS |
| | | | | WGQGTTVTVS | QQGNTLPW | TTPPVLDSDGSFFLYSKLTVDKSRWQQGNVFSCSVM | STLTLSKADYEKHKVY |
| | | | | S | TFGAGTKL | HEALHNHYTQKSLSLSPGK | ACEVTHQGLSSPVTKS |
| | | | | | ELK | | FNRGEC |
| | | | | 711 | 712 | 713 | 714 |
| 22 | CD8B312 | IgG1 | Kappa | QVQLQQPGAD | DIVLTQSP | QVQLQQPGADLVKPGASVKLSCKASGYTFTSFWMHW | DIVLTQSPATLSVTPG |
| | | | | LVKPGASVKL | ATLSVTPG | VKQRPGQGLEWIGNVDPSDSQTHYNQKFKDKATLTV | DSVSLSCRASQSINNN |
| | | | | SCKASGYTFT | DSVSLSCR | DKSSNTAYMQLSSLTSEDSAVYYCARSTYYRYDGPF | LHWYQQKSHESPRLLI |
| | | | | SFWMHWVKQR | ASQSINNN | TYWGQGTTVTVSSASTKGPSVFPLAPSSKSTSGGTA | KYTSQSISGIPSRFSG |
| | | | | PGQGLEWIGN | LHWYQQKS | ALGCLVKDYFPEPVTVSWNSGALTSGVHTFPAVLQS | SGSGPDFTLSINSVET |
| | | | | VDPSDSQTHY | HESPRLLI | SGLYSLSSVVTVPSSSLGTQTYICNVNHKPSNTKVD | EDFGMYFCQQSNSWPL |
| | | | | NQKFKDKATL | KYTSQSIS | KKVEPKSCDKTHTCPPCPAPELLGGPSVFLFPPKPK | TFGGGTKLEIKRTVAA |
| | | | | TVDKSSNTAY | GIPSRFSG | DTLMISRTPEVTCVVVDVSHEDPEVKFNWYVDGVEV | PSVFIFPPSDEQLKSG |
| | | | | MQLSSLTSED | SGSGPDFT | HNAKTKPREEQYNSTYRVVSVLTVLHQDWLNGKEYK | TASVVCLLNNFYPREA |
| | | | | SAVYYCARST | LSINSVET | CKVSNKALPAPIEKTISKAKGQPREPQVYTLPPSRE | KVQWKVDNALQSGNSQ |
| | | | | YYRYDGPFTY | EDFGMYFC | EMTKNQVSLTCLVKGFYPSDIAVEWESNGQPENNYK | ESVTEQDSKDSTYSLS |
| | | | | WGQGTTVTVS | QQSNSWPL | TTPPVLDSDGSFFLYSKLTVDKSRWQQGNVFSCSVM | STLTLSKADYEKHKVY |
| | | | | S | TFGGGTKL | HEALHNHYTQKSLSLSPGK | ACEVTHQGLSSPVTKS |
| | | | | | EIK | | FNRGEC |
| | | | | 745 | 746 | 747 | 748 |
| 23 | CD8B347 | IgG1 | Kappa | QVQLQQPGAE | DIQMTQSP | QVQLQQPGAELAKPGTSVKMSCKASGYTFTSYWMNW | DIQMTQSPASLSASVG |
| | | | | LAKPGTSVKM | ASLSASVG | IKQRPGQGLEWIGAVNPSNSYTEYAQKFKDKAILTA | ETVTITCRASGNIHNY |
| | | | | SCKASGYTFT | ETVTITCR | DKSSSTAYMSLSGLTSEASAVYYCARSGLYNTNHLA | LAWYQQKQGKSPQLLV |
| | | | | SYWMNWIKQR | ASGNIHNY | WFAYWGQGTLVTVSAASTKGPSVFPLAPSSKSTSGG | FNAETLADGVPSRFSG |
| | | | | PGQGLEWIGA | LAWYQQKQ | TAALGCLVKDYFPEPVTVSWNSGALTSGVHTFPAVL | SGSGTQFSLKINSLQP |
| | | | | VNPSNSYTEY | GKSPQLLV | QSSGLYSLSSVVTVPSSSLGTQTYICNVNHKPSNTK | EDFGTYYCQHFWNNPL |
| | | | | AQKFKDKAIL | FNAETLAD | VDKKVEPKSCDKTHTCPPCPAPELLGGPSVFLFPPK | TLGAGTKLELKRTVAA |
| | | | | TADKSSSTAY | GVPSRFSG | PKDTLMISRTPEVTCVVVDVSHEDPEVKFNWYVDGV | PSVFIFPPSDEQLKSG |
| | | | | MSLSGLTSEA | SGSGTQFS | EVHNAKTKPREEQYNSTYRVVSVLTVLHQDWLNGKE | TASVVCLLNNFYPREA |
| | | | | SAVYYCARSG | LKINSLQP | YKCKVSNKALPAPIEKTISKAKGQPREPQVYTLPPS | KVQWKVDNALQSGNSQ |
| | | | | LYNTNHLAWF | EDFGTYYC | REEMTKNQVSLTCLVKGFYPSDIAVEWESNGQPENN | ESVTEQDSKDSTYSLS |
| | | | | AYWGQGTLVT | QHFWNNPL | YKTTPPVLDSDGSFFLYSKLTVDKSRWQQGNVFSCS | STLTLSKADYEKHKVY |
| | | | | VSA | TLGAGTKL | VMHEALHNHYTQKSLSLSPGK | ACEVTHQGLSSPVTKS |
| | | | | | ELK | | FNRGEC |
| | | | | 779 | 780 | 781 | 782 |
| 24 | CD8B350 | IgG1 | Kappa | EVQLQQSGAE | DIVMTQSP | EVQLQQSGAELAKPGTSVKMSCKASGYTFAAYWINW | DIVMTQSPASLSASVG |
| | | | | LAKPGTSVKM | ASLSASVG | LKQRPGQGLEWIGSINPSNGYTEYSQKFKDKAILTA | ETVTITCRASGNIHNY |
| | | | | SCKASGYTFA | ETVTITCR | DKSSSTAYMQLSSLTSEDSAVYYCSRSGLYYTNHLA | LAWYQQKQGKSPQVLV |
| | | | | AYWINWLKQR | ASGNIHNY | WCPYWGQGTTVTVSSASTKGPSVFPLAPSSKSTSGG | YNAETLADSVPSRFSG |
| | | | | PGQGLEWIGS | LAWYQQKQ | TAALGCLVKDYFPEPVTVSWNSGALTSGVHTFPAVL | SGSGTQFSLKINSLQP |
| | | | | INPSNGYTEY | GKSPQVLV | QSSGLYSLSSVVTVPSSSLGTQTYICNVNHKPSNTK | EDFGNYYCQHFWNSPL |
| | | | | SQKFKDKAIL | YNAETLAD | VDKKVEPKSCDKTHTCPPCPAPELLGGPSVFLFPPK | TFGGGTKLEIKRTVAA |
| | | | | TADKSSSTAY | SVPSRFSG | PKDTLMISRTPEVTCVVVDVSHEDPEVKFNWYVDGV | PSVFIFPPSDEQLKSG |
| | | | | MQLSSLTSED | SGSGTQFS | EVHNAKTKPREEQYNSTYRVVSVLTVLHQDWLNGKE | TASVVCLLNNFYPREA |
| | | | | SAVYYCSRSG | LKINSLQP | YKCKVSNKALPAPIEKTISKAKGQPREPQVYTLPPS | KVQWKVDNALQSGNSQ |
| | | | | LYYTNHLAWC | EDFGNYYC | REEMTKNQVSLTCLVKGFYPSDIAVEWESNGQPENN | ESVTEQDSKDSTYSLS |
| | | | | PYWGQGTTVT | QHFWNSPL | YKTTPPVLDSDGSFFLYSKLTVDKSRWQQGNVFSCS | STLTLSKADYEKHKVY |
| | | | | VSS | TFGGGTKL | VMHEALHNHYTQKSLSLSPGK | ACEVTHQGLSSPVTKS |
| | | | | | EIK | | FNRGEC |
| | | | | 813 | 814 | 815 | 816 |
| 25 | CD8B356 | IgG1 | Kappa | DVQLQESGPG | DIVLTQSQ | DVQLQESGPGLVKPSQSLSLTCSVTGYSITSGYYWN | DIVLTQSQKFMSTTVG |
| | | | | LVKPSQSLSL | KFMSTTVG | WIRQFPGNKLEWMGYISYDGSNNYNPSLKNRISITR | DRVSITCKASQNVGTA |
| | | | | TCSVTGYSIT | DRVSITCK | DTSKNQFFLKLNSVTTEDTATYYCVRNHGDAMDYWG | VAWYQQKPGQSPKLLI |
| | | | | SGYYWNWIRQ | ASQNVGTA | QGTSVTVSSASTKGPSVFPLAPSSKSTSGGTAALGC | YSASYRYTGVPDRFTG |
| | | | | FPGNKLEWMG | VAWYQQKP | LVKDYFPEPVTVSWNSGALTSGVHTFPAVLQSSGLY | SGSGTHFTLTISNMQS |
| | | | | YISYDGSNNY | GQSPKLLI | SLSSVVTVPSSSLGTQTYICNVNHKPSNTKVDKKVE | EDLADYFCQQYSSYLT |
| | | | | NPSLKNRISI | YSASYRYT | PKSCDKTHTCPPCPAPELLGGPSVFLFPPKPKDTLM | FGSGTKLEIKRTVAAP |
| | | | | TRDTSKNQFF | GVPDRFTG | ISRTPEVTCVVVDVSHEDPEVKFNWYVDGVEVHNAK | SVFIFPPSDEQLKSGT |
| | | | | LKLNSVTTED | SGSGTHFT | TKPREEQYNSTYRVVSVLTVLHQDWLNGKEYKCKVS | ASVVCLLNNFYPREAK |
| | | | | TATYYCVRNH | LTISNMQS | NKALPAPIEKTISKAKGQPREPQVYTLPPSREEMTK | VQWKVDNALQSGNSQE |
| | | | | GDAMDYWGQG | EDLADYFC | NQVSLTCLVKGFYPSDIAVEWESNGQPENNYKTTPP | SVTEQDSKDSTYSLSS |
| | | | | TSVTVSS | QQYSSYLT | VLDSDGSFFLYSKLTVDKSRWQQGNVFSCSVMHEAL | TLTLSKADYEKHKVYA |
| | | | | | FGSGTKLE | HNHYTQKSLSLSPGK | CEVTHQGLSSPVTKSF |
| | | | | | IK | | NRGEC |
| | | | | 847 | 848 | 849 | 850 |
| 26 | CD8B369 | IgG1 | Kappa | QVQLQQSGAE | DIVMTQSP | QVQLQQSGAELVKPGASVKLSCKTSGFTFTNTYISW | DIVMTQSPASLSASVG |
| | | | | LVKPGASVKL | ASLSASVG | LKQKPRQSLEWIAWIYTGTGGTWYNQKFTDKAQLTV | ETVTITCRASENIYSY |
| | | | | SCKTSGFTFT | ETVTITCR | DTSSSTAYMQVSSLTSEDSAIYYCARTNWDWYFDVW | LAWYQQKQGKSPQLLV |
| | | | | NTYISWLKQK | ASENIYSY | GAGTSVTVSSASTKGPSVFPLAPSSKSTSGGTAALG | YYAKTLTDGVPSRFSG |
| | | | | PRQSLEWIAW | LAWYQQKQ | CLVKDYFPEPVTVSWNSGALTSGVHTFPAVLQSSGL | SGSGTQFSLKINSLQP |
| | | | | IYTGTGGTWY | GKSPQLLV | YSLSSVVTVPSSSLGTQTYICNVNHKPSNTKVDKKV | EDFGSYYCQHHYGRPY |
| | | | | NQKFTDKAQL | YYAKTLTD | EPKSCDKTHTCPPCPAPELLGGPSVFLFPPKPKDTL | TFGSGTKLEIKRTVAA |
| | | | | TVDTSSSTAY | GVPSRFSG | MISRTPEVTCVVVDVSHEDPEVKFNWYVDGVEVHNA | PSVFIFPPSDEQLKSG |
| | | | | MQVSSLTSED | SGSGTQFS | KTKPREEQYNSTYRVVSVLTVLHQDWLNGKEYKCKV | TASVVCLLNNFYPREA |
| | | | | SAIYYCARTN | LKINSLQP | SNKALPAPIEKTISKAKGQPREPQVYTLPPSREEMT | KVQWKVDNALQSGNSQ |
| | | | | WDWYFDVWGA | EDFGSYYC | KNQVSLTCLVKGFYPSDIAVEWESNGQPENNYKTTP | ESVTEQDSKDSTYSLS |
| | | | | GTSVTVSS | QHHYGRPY | PVLDSDGSFFLYSKLTVDKSRWQQGNVFSCSVMHEA | STLTLSKADYEKHKVY |
| | | | | | TFGSGTKL | LHNHYTQKSLSLSPGK | ACEVTHQGLSSPVTKS |
| | | | | | EIK | | FNRGEC |
| | | | | 881 | 882 | 883 | 884 |
| 27 | CD8B371 | IgG1 | Kappa | EVKLVESGGG | NTQMNQTP | EVKLVESGGGLVQPGSSMKLSCTASGFTFSDYYMAW | NTQMNQTPSSLSASLG |
| | | | | LVQPGSSMKL | SSLSASLG | VRQVPEKGLEWVAHINYDGSITYYLDSLKSRFIISR | DTITITCHASQNINVW |
| | | | | SCTASGFTFS | DTITITCH | DNAKNILYLQMSSLKSEDTATYYCAREDYSNYGFAY | LSWYQQKPGNIPKLLI |
| | | | | DYYMAWVRQV | ASQNINVW | WGQGTLVTVSAASTKGPSVFPLAPSSKSTSGGTAAL | YKASNLHTGVPSRFSG |
| | | | | PEKGLEWVAH | LSWYQQKP | GCLVKDYFPEPVTVSWNSGALTSGVHTFPAVLQSSG | SGSGTGFTLTISSLQP |
| | | | | INYDGSITYY | GNIPKLLI | LYSLSSVVTVPSSSLGTQTYICNVNHKPSNTKVDKK | EDIATYYCQQGQSYPL |
| | | | | LDSLKSRFII | YKASNLHT | VEPKSCDKTHTCPPCPAPELLGGPSVFLFPPKPKDT | TFGSGTKLEMKRTVAA |
| | | | | SRDNAKNILY | GVPSRFSG | LMISRTPEVTCVVVDVSHEDPEVKFNWYVDGVEVHN | PSVFIFPPSDEQLKSG |
| | | | | LQMSSLKSED | SGSGTGFT | AKTKPREEQYNSTYRVVSVLTVLHQDWLNGKEYKCK | TASVVCLLNNFYPREA |
| | | | | TATYYCARED | LTISSLQP | VSNKALPAPIEKTISKAKGQPREPQVYTLPPSREEM | KVQWKVDNALQSGNSQ |
| | | | | YSNYGFAYWG | EDIATYYC | TKNQVSLTCLVKGFYPSDIAVEWESNGQPENNYKTT | ESVTEQDSKDSTYSLS |
| | | | | QGTLVTVSA | QQGQSYPL | PPVLDSDGSFFLYSKLTVDKSRWQQGNVFSCSVMHE | STLTLSKADYEKHKVY |
| | | | | | TFGSGTKL | ALHNHYTQKSLSLSPGK | ACEVTHQGLSSPVTKS |
| | | | | | EMK | | FNRGEC |
| | | | | 915 | 916 | 917 | 918 |
| 28 | CD8B182 | IgG1 | Kappa | EVQLQQSGAA | DIKMTQSP | EVQLQQSGAALAKPGTSVKMSCKASGYTFTSYWMNW | DIKMTQSPASLSASVG |
| | | | | LAKPGTSVKM | ASLSASVG | VRQRPGQGLEWIGAVNPTNYYTEYIQKFKDKAILTA | ETVTITCRASENIHNY |
| | | | | SCKASGYTFT | ETVTITCR | DKSSSTAYMHLSGLTSEDSAVYYCARSGLYNTNHLA | LAWYQQIQGKSPQLLV |
| | | | | SYWMNWVRQR | ASENIHNY | WFAYWGQGTTVTVSSASTKGPSVFPLAPSSKSTSGG | YNAKTLANGVPSRFSG |
| | | | | PGQGLEWIGA | LAWYQQIQ | TAALGCLVKDYFPEPVTVSWNSGALTSGVHTFPAVL | SASGTQFSLTINSLQP |
| | | | | VNPTNYYTEY | GKSPQLLV | QSSGLYSLSSVVTVPSSSLGTQTYICNVNHKPSNTK | EDFGSYYCQHFWTTPL |
| | | | | IQKFKDKAIL | YNAKTLAN | VDKKVEPKSCDKTHTCPPCPAPELLGGPSVFLFPPK | TFGAGTKLELKRTVAA |
| | | | | TADKSSSTAY | GVPSRFSG | PKDTLMISRTPEVTCVVVDVSHEDPEVKFNWYVDGV | PSVFIFPPSDEQLKSG |
| | | | | MHLSGLTSED | SASGTQFS | EVHNAKTKPREEQYNSTYRVVSVLTVLHQDWLNGKE | TASVVCLLNNFYPREA |
| | | | | SAVYYCARSG | LTINSLQP | YKCKVSNKALPAPIEKTISKAKGQPREPQVYTLPPS | KVQWKVDNALQSGNSQ |
| | | | | LYNTNHLAWF | EDFGSYYC | REEMTKNQVSLTCLVKGFYPSDIAVEWESNGQPENN | ESVTEQDSKDSTYSLS |
| | | | | AYWGQGTTVT | QHFWTTPL | YKTTPPVLDSDGSFFLYSKLTVDKSRWQQGNVFSCS | STLTLSKADYEKHKVY |
| | | | | VSS | TFGAGTKL | VMHEALHNHYTQKSLSLSPGK | ACEVTHQGLSSPVTKS |
| | | | | | ELK | | FNRGEC |
| | | | | 949 | 950 | 951 | 952 |
| 29 | CD8B205 | IgG1 | Kappa | QVQLQQPGAE | DIQMTQSP | QVQLQQPGAELVKPGASVKLSCKASGYSFNSYWMHW | DIQMTQSPASLSASVG |
| | | | | LVKPGASVKL | ASLSASVG | VKQRPGQGLEWIGNIDPSDSETHYNQKFKDKATLTV | ETVTITCRASENIYSY |
| | | | | SCKASGYSFN | ETVTITCR | DKSSSTAYMQLSSLTSEDSAVYYCARVYYSYYSYDA | LAWYQQKQGKSPQLLV |
| | | | | SYWMHWVKQR | ASENIYSY | TYFDYWGQGTTLTVSSASTKGPSVFPLAPSSKSTSG | YNAKTLAEGVPSRFSG |
| | | | | PGQGLEWIGN | LAWYQQKQ | GTAALGCLVKDYFPEPVTVSWNSGALTSGVHTFPAV | SGSGTQFSLKINSLQP |
| | | | | IDPSDSETHY | GKSPQLLV | LQSSGLYSLSSVVTVPSSSLGTQTYICNVNHKPSNT | EDFGSYYCQHHYTTPL |
| | | | | NQKFKDKATL | YNAKTLAE | KVDKKVEPKSCDKTHTCPPCPAPELLGGPSVFLFPP | TFGGGTKLEIKRTVAA |
| | | | | TVDKSSSTAY | GVPSRFSG | KPKDTLMISRTPEVTCVVVDVSHEDPEVKFNWYVDG | PSVFIFPPSDEQLKSG |
| | | | | MQLSSLTSED | SGSGTQFS | VEVHNAKTKPREEQYNSTYRVVSVLTVLHQDWLNGK | TASVVCLLNNFYPREA |
| | | | | SAVYYCARVY | LKINSLQP | EYKCKVSNKALPAPIEKTISKAKGQPREPQVYTLPP | KVQWKVDNALQSGNSQ |
| | | | | YSYYSYDATY | EDFGSYYC | SREEMTKNQVSLTCLVKGFYPSDIAVEWESNGQPEN | ESVTEQDSKDSTYSLS |
| | | | | FDYWGQGTTL | QHHYTTPL | NYKTTPPVLDSDGSFFLYSKLTVDKSRWQQGNVFSC | STLTLSKADYEKHKVY |
| | | | | TVSS | TFGGGTKL | SVMHEALHNHYTQKSLSLSPGK | ACEVTHQGLSSPVTKS |
| | | | | | EIK | | FNRGEC |
| | | | | 983 | 984 | 985 | 986 |
| 30 | CD8B223 | IgG1 | Kappa | DVQLQESGPI | DIVMTQSQ | DVQLQESGPILVAPSQSLSITCTVSGFSLTSYSVHW | DIVMTQSQKFMSTSVG |
| | | | | LVAPSQSLSI | KFMSTSVG | VRQPPGKGLEWLGVIWAGGSTNYNSAFMSRLTISKD | DRVRVTCKASQNVNTD |
| | | | | TCTVSGFSLT | DRVRVTCK | NSESQVFLKMISLQTDDTAMYYCAKHSYYSFDAFDY | VAWYQQKPGQSPKALI |
| | | | | SYSVHWVRQP | ASQNVNTD | WGQGTTLTVSSASTKGPSVFPLAPSSKSTSGGTAAL | YSASYRYSGVPDRFTG |
| | | | | PGKGLEWLGV | VAWYQQKP | GCLVKDYFPEPVTVSWNSGALTSGVHTFPAVLQSSG | SGSGTDFTLTISNVQS |
| | | | | IWAGGSTNYN | GQSPKALI | LYSLSSVVTVPSSSLGTQTYICNVNHKPSNTKVDKK | EDLAEYFCQQCNSYPL |
| | | | | SAFMSRLTIS | YSASYRYS | VEPKSCDKTHTCPPCPAPELLGGPSVFLFPPKPKDT | TFGAGTKLELKRTVAA |
| | | | | KDNSESQVFL | GVPDRFTG | LMISRTPEVTCVVVDVSHEDPEVKFNWYVDGVEVHN | PSVFIFPPSDEQLKSG |
| | | | | KMISLQTDDT | SGSGTDFT | AKTKPREEQYNSTYRVVSVLTVLHQDWLNGKEYKCK | TASVVCLLNNFYPREA |
| | | | | AMYYCAKHSY | LTISNVQS | VSNKALPAPIEKTISKAKGQPREPQVYTLPPSREEM | KVQWKVDNALQSGNSQ |
| | | | | YSFDAFDYWG | EDLAEYFC | TKNQVSLTCLVKGFYPSDIAVEWESNGQPENNYKTT | ESVTEQDSKDSTYSLS |
| | | | | QGTTLTVSS | QQCNSYPL | PPVLDSDGSFFLYSKLTVDKSRWQQGNVFSCSVMHE | STLTLSKADYEKHKVY |
| | | | | | TFGAGTKL | ALHNHYTQKSLSLSPGK | ACEVTHQGLSSPVTKS |
| | | | | | ELK | | FNRGEC |
| | | | | 1017 | 1018 | 1019 | 1020 |
| 31 | CD8B234 | IgG1 | Kappa | QVQLKESGPG | DIQMTQSS | QVQLKESGPGLVKPSQSLSLTCSVTGYSITSGYYWN | DIQMTQSSSSFSVSLG |
| | | | | LVKPSQSLSL | SSFSVSLG | WIRQFPGNKLEWMGYINYDGRNNYNPSLKNRISITR | DRVTITCKASEDIYNR |
| | | | | TCSVTGYSIT | DRVTITCK | DTSKNHFFLKLNSVTTEDTATYYCSRDQGYSKFYFD | LAWYQQRPGNAPRLLI |
| | | | | SGYYWNWIRQ | ASEDIYNR | YWGQGTTLTVSSASTKGPSVFPLAPSSKSTSGGTAA | SGATSLETGVPSRFSG |
| | | | | FPGNKLEWMG | LAWYQQRP | LGCLVKDYFPEPVTVSWNSGALTSGVHTFPAVLQSS | GGSGKDYTLSITSLQT |
| | | | | YINYDGRNNY | GNAPRLLI | GLYSLSSVVTVPSSSLGTQTYICNVNHKPSNTKVDK | EDVANYYCQQYWSFPR |
| | | | | NPSLKNRISI | SGATSLET | KVEPKSCDKTHTCPPCPAPELLGGPSVFLFPPKPKD | TFGGGTKLEIKRTVAA |
| | | | | TRDTSKNHFF | GVPSRFSG | TLMISRTPEVTCVVVDVSHEDPEVKFNWYVDGVEVH | PSVFIFPPSDEQLKSG |
| | | | | LKLNSVTTED | GGSGKDYT | NAKTKPREEQYNSTYRVVSVLTVLHQDWLNGKEYKC | TASVVCLLNNFYPREA |
| | | | | TATYYCSRDQ | LSITSLQT | KVSNKALPAPIEKTISKAKGQPREPQVYTLPPSREE | KVQWKVDNALQSGNSQ |
| | | | | GYSKFYFDYW | EDVANYYC | MTKNQVSLTCLVKGFYPSDIAVEWESNGQPENNYKT | ESVTEQDSKDSTYSLS |
| | | | | GQGTTLTVSS | QQYWSFPR | TPPVLDSDGSFFLYSKLTVDKSRWQQGNVFSCSVMH | STLTLSKADYEKHKVY |
| | | | | | TFGGGTKL | EALHNHYTQKSLSLSPGK | ACEVTHQGLSSPVTKS |
| | | | | | EIK | | FNRGEC |
| | | | | 1051 | 1052 | 1053 | 1054 |
| 32 | CD8B251 | IgG1 | Kappa | QVQLKGSGPG | DIKMTQSQ | QVQLKGSGPGLVQPSQSLSITCTVSGFSLTTYAVHW | DIKMTQSQKFMSTTVG |
| | | | | LVQPSQSLSI | KFMSTTVG | VRQSPGKGLEWLGVIWSGGSTDYNAAFISRLSISKD | DRVSITCKASQNVGTA |
| | | | | TCTVSGFSLT | DRVSITCK | NSKSQVFFKMNSLQADDTAIYYCARHSYYHYNAMDN | VAWYQQKPGQSPKLLI |
| | | | | TYAVHWVRQS | ASQNVGTA | WGQGTSVTVSSASTKGPSVFPLAPSSKSTSGGTAAL | YSASNRYTGVPDRFTG |
| | | | | PGKGLEWLGV | VAWYQQKP | GCLVKDYFPEPVTVSWNSGALTSGVHTFPAVLQSSG | SGSGTDFTLTISNMQS |
| | | | | IWSGGSTDYN | GQSPKLLI | LYSLSSVVTVPSSSLGTQTYICNVNHKPSNTKVDKK | EDLADYFCQQYSSYPF |
| | | | | AAFISRLSIS | YSASNRYT | VEPKSCDKTHTCPPCPAPELLGGPSVFLFPPKPKDT | TFGSGTKLEIKRTVAA |
| | | | | KDNSKSQVFF | GVPDRFTG | LMISRTPEVTCVVVDVSHEDPEVKFNWYVDGVEVHN | PSVFIFPPSDEQLKSG |
| | | | | KMNSLQADDT | SGSGTDFT | AKTKPREEQYNSTYRVVSVLTVLHQDWLNGKEYKCK | TASVVCLLNNFYPREA |
| | | | | AIYYCARHSY | LTISNMQS | VSNKALPAPIEKTISKAKGQPREPQVYTLPPSREEM | KVQWKVDNALQSGNSQ |
| | | | | YHYNAMDNWG | EDLADYFC | TKNQVSLTCLVKGFYPSDIAVEWESNGQPENNYKTT | ESVTEQDSKDSTYSLS |
| | | | | QGTSVTVSS | QQYSSYPF | PPVLDSDGSFFLYSKLTVDKSRWQQGNVFSCSVMHE | STLTLSKADYEKHKVY |
| | | | | | TFGSGTKL | ALHNHYTQKSLSLSPGK | ACEVTHQGLSSPVTKS |
| | | | | | EIK | | FNRGEC |
| | | | | 1085 | 1086 | 1087 | 1088 |
| 33 | CD8B269 | IgG1 | Kappa | DVQLQESGPG | DIVMTQSQ | DVQLQESGPGLVKPSQSLSLTCSVTGYSITSGYYWN | DIVMTQSQKFMSTSVG |
| | | | | LVKPSQSLSL | KFMSTSVG | WIRQFPGNKLEWMGYISYDGSNNYNPSLKNRISITR | DRVRVTCKASQNVGTD |
| | | | | TCSVTGYSIT | DRVRVTCK | DTSKNQFFLKLNSVTTEDTATYYCVRNHGDAMDHWG | VAWYQQKPGQSPKALI |
| | | | | SGYYWNWIRQ | ASQNVGTD | QGTTLTVSSASTKGPSVFPLAPSSKSTSGGTAALGC | YSASYRYSGVPDRFTG |
| | | | | FPGNKLEWMG | VAWYQQKP | LVKDYFPEPVTVSWNSGALTSGVHTFPAVLQSSGLY | SGSGTDFTLTISDVQS |
| | | | | YISYDGSNNY | GQSPKALI | SLSSVVTVPSSSLGTQTYICNVNHKPSNTKVDKKVE | EDLAEYFCQQYKSYPL |
| | | | | NPSLKNRISI | YSASYRYS | PKSCDKTHTCPPCPAPELLGGPSVFLFPPKPKDTLM | TFGAGTKLELKRTVAA |
| | | | | TRDTSKNQFF | GVPDRFTG | ISRTPEVTCVVVDVSHEDPEVKFNWYVDGVEVHNAK | PSVFIFPPSDEQLKSG |
| | | | | LKLNSVTTED | SGSGTDFT | TKPREEQYNSTYRVVSVLTVLHQDWLNGKEYKCKVS | TASVVCLLNNFYPREA |
| | | | | TATYYCVRNH | LTISDVQS | NKALPAPIEKTISKAKGQPREPQVYTLPPSREEMTK | KVQWKVDNALQSGNSQ |
| | | | | GDAMDHWGQG | EDLAEYFC | NQVSLTCLVKGFYPSDIAVEWESNGQPENNYKTTPP | ESVTEQDSKDSTYSLS |
| | | | | TTLTVSS | QQYKSYPL | VLDSDGSFFLYSKLTVDKSRWQQGNVFSCSVMHEAL | STLTLSKADYEKHKVY |
| | | | | | TFGAGTKL | HNHYTQKSLSLSPGK | ACEVTHQGLSSPVTKS |
| | | | | | ELK | | FNRGEC |
| | | | | 1119 | 1120 | 1121 | 1122 |
| 34 | CD8B290 | IgG1 | Kappa | QVQLKESGPG | DIVMTQSH | QVQLKESGPGLVAPSQSLSITCTVSGFSLSRYSVHW | DIVMTQSHKFMSTSVG |
| | | | | LVAPSQSLSI | KFMSTSVG | VRQPPGKGLVWLGMIWGGGSTDYNSALKSRLSISKD | DRVSITCKASQDVGTV |
| | | | | TCTVSGFSLS | DRVSITCK | NSKSQVFLKMNSLQTDDTAMYYCARIYFDNYVGFAY | VAWYQQKPGQSPKLLI |
| | | | | RYSVHWVRQP | ASQDVGTV | WGQGTTLTVSSASTKGPSVFPLAPSSKSTSGGTAAL | FWTSTRHTGVPDRFTG |
| | | | | PGKGLVWLGM | VAWYQQKP | GCLVKDYFPEPVTVSWNSGALTSGVHTFPAVLQSSG | SGSGTDFTLTISNVQS |
| | | | | IWGGGSTDYN | GQSPKLLI | LYSLSSVVTVPSSSLGTQTYICNVNHKPSNTKVDKK | EDLADYFCQQYSSYPY |
| | | | | SALKSRLSIS | FWTSTRHT | VEPKSCDKTHTCPPCPAPELLGGPSVFLFPPKPKDT | TFGSGTKLELKRTVAA |
| | | | | KDNSKSQVFL | GVPDRFTG | LMISRTPEVTCVVVDVSHEDPEVKFNWYVDGVEVHN | PSVFIFPPSDEQLKSG |
| | | | | KMNSLQTDDT | SGSGTDFT | AKTKPREEQYNSTYRVVSVLTVLHQDWLNGKEYKCK | TASVVCLLNNFYPREA |
| | | | | AMYYCARIYF | LTISNVQS | VSNKALPAPIEKTISKAKGQPREPQVYTLPPSREEM | KVQWKVDNALQSGNSQ |
| | | | | DNYVGFAYWG | EDLADYFC | TKNQVSLTCLVKGFYPSDIAVEWESNGQPENNYKTT | ESVTEQDSKDSTYSLS |
| | | | | QGTTLTVSS | QQYSSYPY | PPVLDSDGSFFLYSKLTVDKSRWQQGNVFSCSVMHE | STLTLSKADYEKHKVY |
| | | | | | TFGSGTKL | ALHNHYTQKSLSLSPGK | ACEVTHQGLSSPVTKS |
| | | | | | ELK | | FNRGEC |
| | | | | 1153 | 1154 | 1155 | 1156 |
| 35 | CD8B310 | IgG1 | Kappa | QVQLKESGPG | DVLMTQTP | QVQLKESGPGLVAPSQSLSITCTVSGFSLTNYAVHW | DVLMTQTPLSLPVSLG |
| | | | | LVAPSQSLSI | LSLPVSLG | VRQSPGKGLEWLGVIWTDGSTDYNAGFISRLSISKD | DQASISCRSSQTIVHS |
| | | | | TCTVSGFSLT | DQASISCR | NSKSQVFFKMNSLQADDTAIYYCARNNGYFPAFFAY | NGNTYLEWYLQKPGQS |
| | | | | NYAVHWVRQS | SSQTIVHS | WGQGTTVTVSSASTKGPSVFPLAPSSKSTSGGTAAL | PKLLMYKVSNRFSGVP |
| | | | | PGKGLEWLGV | NGNTYLEW | GCLVKDYFPEPVTVSWNSGALTSGVHTFPAVLQSSG | DRFGGSGSGTDFTLKI |
| | | | | IWTDGSTDYN | YLQKPGQS | LYSLSSVVTVPSSSLGTQTYICNVNHKPSNTKVDKK | SRVEAEDLGVYYCFQG |
| | | | | AGFISRLSIS | PKLLMYKV | VEPKSCDKTHTCPPCPAPELLGGPSVFLFPPKPKDT | SHAPFTFGSGTKLEIK |
| | | | | KDNSKSQVFF | SNRFSGVP | LMISRTPEVTCVVVDVSHEDPEVKFNWYVDGVEVHN | RTVAAPSVFIFPPSDE |
| | | | | KMNSLQADDT | DRFGGSGS | AKTKPREEQYNSTYRVVSVLTVLHQDWLNGKEYKCK | QLKSGTASVVCLLNNF |
| | | | | AIYYCARNNG | GTDFTLKI | VSNKALPAPIEKTISKAKGQPREPQVYTLPPSREEM | YPREAKVQWKVDNALQ |
| | | | | YFPAFFAYWG | SRVEAEDL | TKNQVSLTCLVKGFYPSDIAVEWESNGQPENNYKTT | SGNSQESVTEQDSKDS |
| | | | | QGTTVTVSS | GVYYCFQG | PPVLDSDGSFFLYSKLTVDKSRWQQGNVFSCSVMHE | TYSLSSTLTLSKADYE |
| | | | | | SHAPFTFG | ALHNHYTQKSLSLSPGK | KHKVYACEVTHQGLSS |
| | | | | | SGTKLEIK | | PVTKSFNRGEC |
| | | | | 1187 | 1188 | 1189 | 1190 |
| 36 | CD8B352 | IgG1 | Kappa | QVQLKESGPG | DIQMTQSS | QVQLKESGPGLVKPSQSLSLTCSVTGYSITSGYYWN | DIQMTQSSSSFSVSLG |
| | | | | LVKPSQSLSL | SSFSVSLG | WIRQFPGNKLEWMGYINYDGRNNYNPSLRNRISITR | DRVTITCKASEDIYNR |
| | | | | TCSVTGYSIT | DRVTITCK | DTSKNHFFLKLNSVTTEDTATYYCARDQGYSKFYFD | LAWYQQRPGNAPRLLI |
| | | | | SGYYWNWIRQ | ASEDIYNR | YWGQGTTLTVSSASTKGPSVFPLAPSSKSTSGGTAA | SGATSLETGVPSRFSG |
| | | | | FPGNKLEWMG | LAWYQQRP | LGCLVKDYFPEPVTVSWNSGALTSGVHTFPAVLQSS | SGSGKDYTLSITSLQT |
| | | | | YINYDGRNNY | GNAPRLLI | GLYSLSSVVTVPSSSLGTQTYICNVNHKPSNTKVDK | EDVANYYCQQYWSFPR |
| | | | | NPSLRNRISI | SGATSLET | KVEPKSCDKTHTCPPCPAPELLGGPSVFLFPPKPKD | TFGGGTKLEIKRTVAA |
| | | | | TRDTSKNHFF | GVPSRFSG | TLMISRTPEVTCVVVDVSHEDPEVKFNWYVDGVEVH | PSVFIFPPSDEQLKSG |
| | | | | LKLNSVTTED | SGSGKDYT | NAKTKPREEQYNSTYRVVSVLTVLHQDWLNGKEYKC | TASVVCLLNNFYPREA |
| | | | | TATYYCARDQ | LSITSLQT | KVSNKALPAPIEKTISKAKGQPREPQVYTLPPSREE | KVQWKVDNALQSGNSQ |
| | | | | GYSKFYFDYW | EDVANYYC | MTKNQVSLTCLVKGFYPSDIAVEWESNGQPENNYKT | ESVTEQDSKDSTYSLS |
| | | | | GQGTTLTVSS | QQYWSFPR | TPPVLDSDGSFFLYSKLTVDKSRWQQGNVFSCSVMH | STLTLSKADYEKHKVY |
| | | | | | TFGGGTKL | EALHNHYTQKSLSLSPGK | ACEVTHQGLSSPVTKS |
| | | | | | EIK | | FNRGEC |
| | | | | 1221 | 1222 | 1223 | 1224 |
| 37 | CD8B319 | IgG1 | Kappa | QVQLKESGPE | DIVMTQSQ | QVQLKESGPELKKPGETVKISCKASGYSFTAYYMHW | DIVMTQSQKFMSTTVG |
| | | | | LKKPGETVKI | KFMSTTVG | VKQSPEKSLEWIGEINPSAGGTTYNQKFKAKATLTV | DRVSITCKASQNVGTA |
| | | | | SCKASGYSFT | DRVSITCK | DKSSSTAFIQLKSLTSEDSAVYYCARWTNPFDYWGQ | VAWYQQKPGQSPKLLI |
| | | | | AYYMHWVKQS | ASQNVGTA | GTTLTVSSASTKGPSVFPLAPSSKSTSGGTAALGCL | YSASYRYTGVPDRFTG |
| | | | | PEKSLEWIGE | VAWYQQKP | VKDYFPEPVTVSWNSGALTSGVHTFPAVLQSSGLYS | SGSGTHFTLTISNIQS |
| | | | | INPSAGGTTY | GQSPKLLI | LSSVVTVPSSSLGTQTYICNVNHKPSNTKVDKKVEP | EDLADYFCQQYNNYLT |
| | | | | NQKFKAKATL | YSASYRYT | KSCDKTHTCPPCPAPELLGGPSVFLFPPKPKDTLMI | FGSGTKLEIKRTVAAP |
| | | | | TVDKSSSTAF | GVPDRFTG | SRTPEVTCVVVDVSHEDPEVKFNWYVDGVEVHNAKT | SVFIFPPSDEQLKSGT |
| | | | | IQLKSLTSED | SGSGTHFT | KPREEQYNSTYRVVSVLTVLHQDWLNGKEYKCKVSN | ASVVCLLNNFYPREAK |
| | | | | SAVYYCARWT | LTISNIQS | KALPAPIEKTISKAKGQPREPQVYTLPPSREEMTKN | VQWKVDNALQSGNSQE |
| | | | | NPFDYWGQGT | EDLADYFC | QVSLTCLVKGFYPSDIAVEWESNGQPENNYKTTPPV | SVTEQDSKDSTYSLSS |
| | | | | TLTVSS | QQYNNYLT | LDSDGSFFLYSKLTVDKSRWQQGNVFSCSVMHEALH | TLTLSKADYEKHKVYA |
| | | | | | FGSGTKLE | NHYTQKSLSLSPGK | CEVTHQGLSSPVTKSF |
| | | | | | IK | | NRGEC |
| | | | | 1255 | 1256 | 1257 | 1258 |
| 38 | CD8B194 | IgG1 | Kappa | QVQLQQPGAE | DIVMTQSQ | QVQLQQPGAELVKPGASVKLSCKASGYTFTSYWINW | DIVMTQSQKFMSTTVG |
| | | | | LVKPGASVKL | KFMSTTVG | VKQRPGQGLEWIGNIYPGSSSTNYNEKFKSKATLTV | DRVSITCKASQNVGTA |
| | | | | SCKASGYTFT | DRVSITCK | DTSSSAAYMQLSSLTSGDSAVYYCARELGPYYRYSA | VAWYQQKPGQSPKLLI |
| | | | | SYWINWVKQR | ASQNVGTA | MVYWGQGTTVTVSSASTKGPSVFPLAPSSKSTSGGT | YSASNRYTGVPDRFTG |
| | | | | PGQGLEWIGN | VAWYQQKP | AALGCLVKDYFPEPVTVSWNSGALTSGVHTFPAVLQ | SGSGTDFTLTISNMQS |
| | | | | IYPGSSSTNY | GQSPKLLI | SSGLYSLSSVVTVPSSSLGTQTYICNVNHKPSNTKV | EDLADYFCQQYSSYPF |
| | | | | NEKFKSKATL | YSASNRYT | DKKVEPKSCDKTHTCPPCPAPELLGGPSVFLFPPKP | TFGSGTKLEIKRTVAA |
| | | | | TVDTSSSAAY | GVPDRFTG | KDTLMISRTPEVTCVVVDVSHEDPEVKFNWYVDGVE | PSVFIFPPSDEQLKSG |
| | | | | MQLSSLTSGD | SGSGTDFT | VHNAKTKPREEQYNSTYRVVSVLTVLHQDWLNGKEY | TASVVCLLNNFYPREA |
| | | | | SAVYYCAREL | LTISNMQS | KCKVSNKALPAPIEKTISKAKGQPREPQVYTLPPSR | KVQWKVDNALQSGNSQ |
| | | | | GPYYRYSAMV | EDLADYFC | EEMTKNQVSLTCLVKGFYPSDIAVEWESNGQPENNY | ESVTEQDSKDSTYSLS |
| | | | | YWGQGTTVTV | QQYSSYPF | KTTPPVLDSDGSFFLYSKLTVDKSRWQQGNVFSCSV | STLTLSKADYEKHKVY |
| | | | | SS | TFGSGTKL | MHEALHNHYTQKSLSLSPGK | ACEVTHQGLSSPVTKS |
| | | | | | EIK | | FNRGEC |
| | | | | 1289 | 1290 | 1291 | 1292 |
| 39 | CD8B231 | IgG1 | Kappa | EVKLVESGAE | DIQMTQTT | EVKLVESGAELVKPGASVKLSCKASGYTFTNYWMHW | DIQMTQTTSSLSASLG |
| | | | | LVKPGASVKL | SSLSASLG | VKQRPGQGLEWIGNIDPSDSETHYNQKFKDKATLTV | DRVTITCRASQDINIY |
| | | | | SCKASGYTFT | DRVTITCR | DKSSSTAYMQLSSLTSEDSAVYYCASGLTGTGHYWG | LNWYQQKPEGSIKCLI |
| | | | | NYWMHWVKQR | ASQDINIY | QGTTLTVSSASTKGPSVFPLAPSSKSTSGGTAALGC | YHTSRLHSGVPSRFSG |
| | | | | PGQGLEWIGN | LNWYQQKP | LVKDYFPEPVTVSWNSGALTSGVHTFPAVLQSSGLY | SGSGTDYSLTISNLEQ |
| | | | | IDPSDSETHY | EGSIKCLI | SLSSVVTVPSSSLGTQTYICNVNHKPSNTKVDKKVE | EDIATYFCQQDNTLPY |
| | | | | NQKFKDKATL | YHTSRLHS | PKSCDKTHTCPPCPAPELLGGPSVFLFPPKPKDTLM | TFGSGTKLEIKRTVAA |
| | | | | TVDKSSSTAY | GVPSRFSG | ISRTPEVTCVVVDVSHEDPEVKFNWYVDGVEVHNAK | PSVFIFPPSDEQLKSG |
| | | | | MQLSSLTSED | SGSGTDYS | TKPREEQYNSTYRVVSVLTVLHQDWLNGKEYKCKVS | TASVVCLLNNFYPREA |
| | | | | SAVYYCASGL | LTISNLEQ | NKALPAPIEKTISKAKGQPREPQVYTLPPSREEMTK | KVQWKVDNALQSGNSQ |
| | | | | TGTGHYWGQG | EDIATYFC | NQVSLTCLVKGFYPSDIAVEWESNGQPENNYKTTPP | ESVTEQDSKDSTYSLS |
| | | | | TTLTVSS | QQDNTLPY | VLDSDGSFFLYSKLTVDKSRWQQGNVFSCSVMHEAL | STLTLSKADYEKHKVY |
| | | | | | TFGSGTKL | HNHYTQKSLSLSPGK | ACEVTHQGLSSPVTKS |
| | | | | | EIK | | FNRGEC |
| | | | | 1323 | 1324 | 1325 | 1326 |
| 40 | CD8B238 | IgG1 | Kappa | EFQLQQSGPE | DIKMTQSP | EFQLQQSGPELVKPGASLKISCKASGYTFTDYSMDW | DIKMTQSPSSMCPSLG |
| | | | | LVKPGASLKI | SSMCPSLG | VKQSHGKTLEWIGYIYTYSGGAGYNRKFKSKATLTV | ERVTITCKASQDIKSY |
| | | | | SCKASGYTFT | ERVTITCK | DKSSSTAYLELHSLTSDDSAVYYCARDSSDYEFAYW | LSWFQQKPGKSPKTLI |
| | | | | DYSMDWVKQS | ASQDIKSY | GQGTLVTVSAASTKGPSVFPLAPSSKSTSGGTAALG | YRANRLVDGVPSRFSG |
| | | | | HGKTLEWIGY | LSWFQQKP | CLVKDYFPEPVTVSWNSGALTSGVHTFPAVLQSSGL | SGSGQDYSLTISSLEY |
| | | | | IYTYSGGAGY | GKSPKTLI | YSLSSVVTVPSSSLGTQTYICNVNHKPSNTKVDKKV | EDMGIYYCLQYDEFRT |
| | | | | NRKFKSKATL | YRANRLVD | EPKSCDKTHTCPPCPAPELLGGPSVFLFPPKPKDTL | FGGGTKLEIKRTVAAP |
| | | | | TVDKSSSTAY | GVPSRFSG | MISRTPEVTCVVVDVSHEDPEVKFNWYVDGVEVHNA | SVFIFPPSDEQLKSGT |
| | | | | LELHSLTSDD | SGSGQDYS | KTKPREEQYNSTYRVVSVLTVLHQDWLNGKEYKCKV | ASVVCLLNNFYPREAK |
| | | | | SAVYYCARDS | LTISSLEY | SNKALPAPIEKTISKAKGQPREPQVYTLPPSREEMT | VQWKVDNALQSGNSQE |
| | | | | SDYEFAYWGQ | EDMGIYYC | KNQVSLTCLVKGFYPSDIAVEWESNGQPENNYKTTP | SVTEQDSKDSTYSLSS |
| | | | | GTLVTVSA | LQYDEFRT | PVLDSDGSFFLYSKLTVDKSRWQQGNVFSCSVMHEA | TLTLSKADYEKHKVYA |
| | | | | | FGGGTKLE | LHNHYTQKSLSLSPGK | CEVTHQGLSSPVTKSF |
| | | | | | IK | | NRGEC |
| | | | | 1357 | 1358 | 1359 | 1360 |
| 41 | CD8B255 | IgG1 | Kappa | QVTLKESGPG | DIQMTQSP | QVTLKESGPGILQPSQTLSLTCSFSGFSLNTSGMGV | DIQMTQSPASLSVSVG |
| | | | | ILQPSQTLSL | ASLSVSVG | SWIRKPSGKGLEWLAHIFWDDDKRYNPSLKSRLTIS | ETVTITCRASENIYSD |
| | | | | TCSFSGFSLN | ETVTITCR | KDTSSNQVFLMITSVDTADTATYYCARRDGYGDYAY | LAWYQQKQGKSPQLLV |
| | | | | TSGMGVSWIR | ASENIYSD | FDVWGAGTLVTVSAASTKGPSVFPLAPSSKSTSGGT | YAATILTDGVPSRFSG |
| | | | | KPSGKGLEWL | LAWYQQKQ | AALGCLVKDYFPEPVTVSWNSGALTSGVHTFPAVLQ | SGSGTQYSLKINSLQS |
| | | | | AHIFWDDDKR | GKSPQLLV | SSGLYSLSSVVTVPSSSLGTQTYICNVNHKPSNTKV | EDFGNYYCQHFWGTPW |
| | | | | YNPSLKSRLT | YAATILTD | DKKVEPKSCDKTHTCPPCPAPELLGGPSVFLFPPKP | TFGDGTRLEIKRTVAA |
| | | | | ISKDTSSNQV | GVPSRFSG | KDTLMISRTPEVTCVVVDVSHEDPEVKFNWYVDGVE | PSVFIFPPSDEQLKSG |
| | | | | FLMITSVDTA | SGSGTQYS | VHNAKTKPREEQYNSTYRVVSVLTVLHQDWLNGKEY | TASVVCLLNNFYPREA |
| | | | | DTATYYCARR | LKINSLQS | KCKVSNKALPAPIEKTISKAKGQPREPQVYTLPPSR | KVQWKVDNALQSGNSQ |
| | | | | DGYGDYAYFD | EDFGNYYC | EEMTKNQVSLTCLVKGFYPSDIAVEWESNGQPENNY | ESVTEQDSKDSTYSLS |
| | | | | VWGAGTLVTV | QHFWGTPW | KTTPPVLDSDGSFFLYSKLTVDKSRWQQGNVFSCSV | STLTLSKADYEKHKVY |
| | | | | SA | TFGDGTRL | MHEALHNHYTQKSLSLSPGK | ACEVTHQGLSSPVTKS |
| | | | | | EIK | | FNRGEC |
| | | | | 1391 | 1392 | 1393 | 1394 |
| 42 | CD8B324 | IgG1 | Kappa | QVQLQQPGAD | DIVMTQSQ | QVQLQQPGADLVKPGASVKLSCKASGYTSTSHWIHW | DIVMTQSQKFMPTTVG |
| | | | | LVKPGASVKL | KFMPTTVG | VKQRPGQGLEWIGNIYPGSSSTNYNEKFKRMATLTV | DRVSITCKASQNVGTA |
| | | | | SCKASGYTST | DRVSITCK | DTSSSTVYMVLSSLTSDDSAVYYCARHSPGHRDYAM | VAWYQQKPGQSPKLLI |
| | | | | SHWIHWVKQR | ASQNVGTA | DYWGLGTSVTVSSASTKGPSVFPLAPSSKSTSGGTA | ASASNRYTGVPDRFTG |
| | | | | PGQGLEWIGN | VAWYQQKP | ALGCLVKDYFPEPVTVSWNSGALTSGVHTFPAVLQS | SGSGTDFTLTISTMQS |
| | | | | IYPGSSSTNY | GQSPKLLI | SGLYSLSSVVTVPSSSLGTQTYICNVNHKPSNTKVD | EDLADYFCQQYSTYPL |
| | | | | NEKFKRMATL | ASASNRYT | KKVEPKSCDKTHTCPPCPAPELLGGPSVFLFPPKPK | TFGAGTKLEMKRTVAA |
| | | | | TVDTSSSTVY | GVPDRFTG | DTLMISRTPEVTCVVVDVSHEDPEVKFNWYVDGVEV | PSVFIFPPSDEQLKSG |
| | | | | MVLSSLTSDD | SGSGTDFT | HNAKTKPREEQYNSTYRVVSVLTVLHQDWLNGKEYK | TASVVCLLNNFYPREA |
| | | | | SAVYYCARHS | LTISTMQS | CKVSNKALPAPIEKTISKAKGQPREPQVYTLPPSRE | KVQWKVDNALQSGNSQ |
| | | | | PGHRDYAMDY | EDLADYFC | EMTKNQVSLTCLVKGFYPSDIAVEWESNGQPENNYK | ESVTEQDSKDSTYSLS |
| | | | | WGLGTSVTVS | QQYSTYPL | TTPPVLDSDGSFFLYSKLTVDKSRWQQGNVFSCSVM | STLTLSKADYEKHKVY |
| | | | | S | TFGAGTKL | HEALHNHYTQKSLSLSPGK | ACEVTHQGLSSPVTKS |
| | | | | | EMK | | FNRGEC |
| | | | | 1425 | 1426 | 1427 | 1428 |
| 43 | CD8B337 | IgG1 | Kappa | QVTLKESGPG | DIQMTQYP | QVTLKESGPGKVQPSQTLSLTCSFSGFSLSTSGMGV | DIQMTQYPASLSVSVG |
| | | | | KVQPSQTLSL | ASLSVSVG | SWIRKPSGKGLEWLAHIFWDDDRRYKSSLKSRLTIS | ETVTITCRASENIYSD |
| | | | | TCSFSGFSLS | ETVTITCR | KDTSSNQVFLMITSVDTADSATYYCARRVGYGDYAY | LAWYQQKQGKSPQLLV |
| | | | | TSGMGVSWIR | ASENIYSD | FDVWGAGTTVTVSSASTKGPSVFPLAPSSKSTSGGT | YAATNLADGVPSRFSG |
| | | | | KPSGKGLEWL | LAWYQQKQ | AALGCLVKDYFPEPVTVSWNSGALTSGVHTFPAVLQ | SGSGTQYSLKINSLQS |
| | | | | AHIFWDDDRR | GKSPQLLV | SSGLYSLSSVVTVPSSSLGTQTYICNVNHKPSNTKV | EDFGNYYCQHFWGTPW |
| | | | | YKSSLKSRLT | YAATNLAD | DKKVEPKSCDKTHTCPPCPAPELLGGPSVFLFPPKP | TFGGGTKLEIKRTVAA |
| | | | | ISKDTSSNQV | GVPSRFSG | KDTLMISRTPEVTCVVVDVSHEDPEVKFNWYVDGVE | PSVFIFPPSDEQLKSG |
| | | | | FLMITSVDTA | SGSGTQYS | VHNAKTKPREEQYNSTYRVVSVLTVLHQDWLNGKEY | TASVVCLLNNFYPREA |
| | | | | DSATYYCARR | LKINSLQS | KCKVSNKALPAPIEKTISKAKGQPREPQVYTLPPSR | KVQWKVDNALQSGNSQ |
| | | | | VGYGDYAYFD | EDFGNYYC | EEMTKNQVSLTCLVKGFYPSDIAVEWESNGQPENNY | ESVTEQDSKDSTYSLS |
| | | | | VWGAGTTVTV | QHFWGTPW | KTTPPVLDSDGSFFLYSKLTVDKSRWQQGNVFSCSV | STLTLSKADYEKHKVY |
| | | | | SS | TFGGGTKL | MHEALHNHYTQKSLSLSPGK | ACEVTHQGLSSPVTKS |
| | | | | | EIK | | FNRGEC |
| | | | | 1459 | 1460 | 1461 | 1462 |
| 44 | CD8B344 | IgG1 | Kappa | QVQLQQSGAE | DIKMTQSQ | QVQLQQSGAELVKPGASVKLSCKASGYSFTNYWINW | DIKMTQSQKFMSTTVG |
| | | | | LVKPGASVKL | KFMSTTVG | MKQRPGQGLEWIGNIYPGSDSSNYNEKFKTKATLTV | DRVSITCKASQNVGTA |
| | | | | SCKASGYSFT | DRVSITCK | DTSSSTAYMQLSSLTSDDSAVYYCAREEADYRYTWF | VAWYQQKPGQSPKLLI |
| | | | | NYWINWMKQR | ASQNVGTA | VYWGQGTLVTVSAASTKGPSVFPLAPSSKSTSGGTA | YSASNRYTGVPDRFTG |
| | | | | PGQGLEWIGN | VAWYQQKP | ALGCLVKDYFPEPVTVSWNSGALTSGVHTFPAVLQS | SGSGTDFTLTFSNMQS |
| | | | | IYPGSDSSNY | GQSPKLLI | SGLYSLSSVVTVPSSSLGTQTYICNVNHKPSNTKVD | EDLADYFCQQYSSYPL |
| | | | | NEKFKTKATL | YSASNRYT | KKVEPKSCDKTHTCPPCPAPELLGGPSVFLFPPKPK | TFGAGTKLEMKRTVAA |
| | | | | TVDTSSSTAY | GVPDRFTG | DTLMISRTPEVTCVVVDVSHEDPEVKFNWYVDGVEV | PSVFIFPPSDEQLKSG |
| | | | | MQLSSLTSDD | SGSGTDFT | HNAKTKPREEQYNSTYRVVSVLTVLHQDWLNGKEYK | TASVVCLLNNFYPREA |
| | | | | SAVYYCAREE | LTFSNMQS | CKVSNKALPAPIEKTISKAKGQPREPQVYTLPPSRE | KVQWKVDNALQSGNSQ |
| | | | | ADYRYTWFVY | EDLADYFC | EMTKNQVSLTCLVKGFYPSDIAVEWESNGQPENNYK | ESVTEQDSKDSTYSLS |
| | | | | WGQGTLVTVS | QQYSSYPL | TTPPVLDSDGSFFLYSKLTVDKSRWQQGNVFSCSVM | STLTLSKADYEKHKVY |
| | | | | A | TFGAGTKL | HEALHNHYTQKSLSLSPGK | ACEVTHQGLSSPVTKS |
| | | | | | EMK | | FNRGEC |
| | | | | 1493 | 1494 | 1495 | 1496 |
| 45 | CD8B264 | IgG1 | Kappa | EVQLQQSGTE | DIVMTQSQ | EVQLQQSGTELVKPGASVKLSCKASGYSFTSYWINW | DIVMTQSQKFMSTTVG |
| | | | | LVKPGASVKL | KFMSTTVG | VKQRPGQGPEWIGNIYPGSSSTNYNEKFKNKATLTV | DRVSITCKASQNVGTA |
| | | | | SCKASGYSFT | DRVSITCK | DTSSSTAYMQLSSLTSDDSAVYYCAREEYSYKSSWF | VAWYQQKPGQSPKLLI |
| | | | | SYWINWVKQR | ASQNVGTA | AYWGQGTLVTVSAASTKGPSVFPLAPSSKSTSGGTA | YSASNRYNGVPDRFTG |
| | | | | PGQGPEWIGN | VAWYQQKP | ALGCLVKDYFPEPVTVSWNSGALTSGVHTFPAVLQS | SGSGTDFTLTISNMQS |
| | | | | IYPGSSSTNY | GQSPKLLI | SGLYSLSSVVTVPSSSLGTQTYICNVNHKPSNTKVD | EDLADYFCQQYSTYPY |
| | | | | NEKFKNKATL | YSASNRYN | KKVEPKSCDKTHTCPPCPAPELLGGPSVFLFPPKPK | TFGSGTKLEIKRTVAA |
| | | | | TVDTSSSTAY | GVPDRFTG | DTLMISRTPEVTCVVVDVSHEDPEVKFNWYVDGVEV | PSVFIFPPSDEQLKSG |
| | | | | MQLSSLTSDD | SGSGTDFT | HNAKTKPREEQYNSTYRVVSVLTVLHQDWLNGKEYK | TASVVCLLNNFYPREA |
| | | | | SAVYYCAREE | LTISNMQS | CKVSNKALPAPIEKTISKAKGQPREPQVYTLPPSRE | KVQWKVDNALQSGNSQ |
| | | | | YSYKSSWFAY | EDLADYFC | EMTKNQVSLTCLVKGFYPSDIAVEWESNGQPENNYK | ESVTEQDSKDSTYSLS |
| | | | | WGQGTLVTVS | QQYSTYPY | TTPPVLDSDGSFFLYSKLTVDKSRWQQGNVFSCSVM | STLTLSKADYEKHKVY |
| | | | | A | TFGSGTKL | HEALHNHYTQKSLSLSPGK | ACEVTHQGLSSPVTKS |
| | | | | | EIK | | FNRGEC |
| | | | | 1527 | 1528 | 1529 | 1530 |
| 46 | CD8B318 | IgG1 | Kappa | EVQLQQSGAE | DIVMTQSQ | EVQLQQSGAELVKPGASVKLSCKASGYTFTSYWISW | DIVMTQSQKFMSTTIG |
| | | | | LVKPGASVKL | KFMSTTIG | VKQRPGQGLEWIGNIYPGSSSSNYNENFKSKATLTV | DRVSITCKASQNVGTA |
| | | | | SCKASGYTFT | DRVSITCK | DTSSSTAHMQLSSLTSDDSAVFYCAREEYSYFPSWF | VAWFQQKPGQSPKLLI |
| | | | | SYWISWVKQR | ASQNVGTA | AYWGQGTSVTVSSASTKGPSVFPLAPSSKSTSGGTA | YSASNRYTGVPDRFTG |
| | | | | PGQGLEWIGN | VAWFQQKP | ALGCLVKDYFPEPVTVSWNSGALTSGVHTFPAVLQS | SGSGTDFTLTISNMQS |
| | | | | IYPGSSSSNY | GQSPKLLI | SGLYSLSSVVTVPSSSLGTQTYICNVNHKPSNTKVD | EDLANYFCQQYSTYPF |
| | | | | NENFKSKATL | YSASNRYT | KKVEPKSCDKTHTCPPCPAPELLGGPSVFLFPPKPK | TFGGGTKLEIKRTVAA |
| | | | | TVDTSSSTAH | GVPDRFTG | DTLMISRTPEVTCVVVDVSHEDPEVKFNWYVDGVEV | PSVFIFPPSDEQLKSG |
| | | | | MQLSSLTSDD | SGSGTDFT | HNAKTKPREEQYNSTYRVVSVLTVLHQDWLNGKEYK | TASVVCLLNNFYPREA |
| | | | | SAVFYCAREE | LTISNMQS | CKVSNKALPAPIEKTISKAKGQPREPQVYTLPPSRE | KVQWKVDNALQSGNSQ |
| | | | | YSYFPSWFAY | EDLANYFC | EMTKNQVSLTCLVKGFYPSDIAVEWESNGQPENNYK | ESVTEQDSKDSTYSLS |
| | | | | WGQGTSVTVS | QQYSTYPF | TTPPVLDSDGSFFLYSKLTVDKSRWQQGNVFSCSVM | STLTLSKADYEKHKVY |
| | | | | S | TFGGGTKL | HEALHNHYTQKSLSLSPGK | ACEVTHQGLSSPVTKS |
| | | | | | EIK | | FNRGEC |
| | | | | 1561 | 1562 | 1563 | 1564 |
| 47 | CD8B333 | IgG1 | Kappa | QVQLQQPGTE | DIVMTQSQ | QVQLQQPGTELVKPGASVKLSCKASGYSFASFWINW | DIVMTQSQKFMSTTVG |
| | | | | LVKPGASVKL | KFMSTTVG | VKQRPGQGPEWIGNIYPGSSSTNYSEKFKNKATLTV | DRVSITCKASQNVGTA |
| | | | | SCKASGYSFA | DRVSITCK | DKSSSTAYMQLSSLTSDDSAVYYCAREEYSYKSSWF | VAWYQQKPGQSPKLLI |
| | | | | SFWINWVKQR | ASQNVGTA | AYWGQGTTVTVSSASTKGPSVFPLAPSSKSTSGGTA | YSASNRYNGVPDRFTG |
| | | | | PGQGPEWIGN | VAWYQQKP | ALGCLVKDYFPEPVTVSWNSGALTSGVHTFPAVLQS | SGSGTDFTLTISNMQS |
| | | | | IYPGSSSTNY | GQSPKLLI | SGLYSLSSVVTVPSSSLGTQTYICNVNHKPSNTKVD | EDLADYFCQQYSTYPY |
| | | | | SEKFKNKATL | YSASNRYN | KKVEPKSCDKTHTCPPCPAPELLGGPSVFLFPPKPK | TFGSGTKLELKRTVAA |
| | | | | TVDKSSSTAY | GVPDRFTG | DTLMISRTPEVTCVVVDVSHEDPEVKFNWYVDGVEV | PSVFIFPPSDEQLKSG |
| | | | | MQLSSLTSDD | SGSGTDFT | HNAKTKPREEQYNSTYRVVSVLTVLHQDWLNGKEYK | TASVVCLLNNFYPREA |
| | | | | SAVYYCAREE | LTISNMQS | CKVSNKALPAPIEKTISKAKGQPREPQVYTLPPSRE | KVQWKVDNALQSGNSQ |
| | | | | YSYKSSWFAY | EDLADYFC | EMTKNQVSLTCLVKGFYPSDIAVEWESNGQPENNYK | ESVTEQDSKDSTYSLS |
| | | | | WGQGTTVTVS | QQYSTYPY | TTPPVLDSDGSFFLYSKLTVDKSRWQQGNVFSCSVM | STLTLSKADYEKHKVY |
| | | | | S | TFGSGTKL | HEALHNHYTQKSLSLSPGK | ACEVTHQGLSSPVTKS |
| | | | | | ELK | | FNRGEC |
| | | | | 1595 | 1596 | 1597 | 1598 |
| 48 | CD8B366 | IgG1 | Kappa | EVQLQQSGPE | DIKMTQSP | EVQLQQSGPELVRPGASVKLSCTASGFNIKDDYIHW | DIKMTQSPSYLAASPG |
| | | | | LVRPGASVKL | SYLAASPG | VKQRPEQGLEWIGRIDPANGNPRYAPKFQDKATLTA | ETITINCRASKSISKY |
| | | | | SCTASGFNIK | ETITINCR | DTSSNTAYLQLSSLTSEDTAVYYCARDDEGYYYFDV | LAWYQEKPGKTNKVLI |
| | | | | DDYIHWVKQR | ASKSISKY | WGAGTSVTVSSASTKGPSVFPLAPSSKSTSGGTAAL | YSGSTLQSGIPSRFSG |
| | | | | PEQGLEWIGR | LAWYQEKP | GCLVKDYFPEPVTVSWNSGALTSGVHTFPAVLQSSG | SGSGTDFTLTISSLEP |
| | | | | IDPANGNPRY | GKTNKVLI | LYSLSSVVTVPSSSLGTQTYICNVNHKPSNTKVDKK | EDFAIYYCQQHNEYPL |
| | | | | APKFQDKATL | YSGSTLQS | VEPKSCDKTHTCPPCPAPELLGGPSVFLFPPKPKDT | TFGDGTRLEIKRTVAA |
| | | | | TADTSSNTAY | GIPSRFSG | LMISRTPEVTCVVVDVSHEDPEVKFNWYVDGVEVHN | PSVFIFPPSDEQLKSG |
| | | | | LQLSSLTSED | SGSGTDFT | AKTKPREEQYNSTYRVVSVLTVLHQDWLNGKEYKCK | TASVVCLLNNFYPREA |
| | | | | TAVYYCARDD | LTISSLEP | VSNKALPAPIEKTISKAKGQPREPQVYTLPPSREEM | KVQWKVDNALQSGNSQ |
| | | | | EGYYYFDVWG | EDFAIYYC | TKNQVSLTCLVKGFYPSDIAVEWESNGQPENNYKTT | ESVTEQDSKDSTYSLS |
| | | | | AGTSVTVSS | QQHNEYPL | PPVLDSDGSFFLYSKLTVDKSRWQQGNVFSCSVMHE | STLTLSKADYEKHKVY |
| | | | | | TFGDGTRL | ALHNHYTQKSLSLSPGK | ACEVTHQGLSSPVTKS |
| | | | | | EIK | | FNRGEC |
| | | | | 1629 | 1630 | 1631 | 1632 |
| 49 | CD8B368 | IgG1 | Kappa | QVQLQQPGTE | DIVMTQSQ | QVQLQQPGTELVKPGASVKLSCKASGYTFTSYWINW | DIVMTQSQKFMSTTVG |
| | | | | LVKPGASVKL | KFMSTTVG | MKQRPGQGLEWIGNIYPFSSSTNYNEKFKKKATLTV | DRVSITCKASQNVGIA |
| | | | | SCKASGYTFT | DRVSITCK | DASSSTASMQLSSLTSDDSAVYFCAREEFSHYPSWF | VAWFQQKPGQSPKLLI |
| | | | | SYWINWMKQR | ASQNVGIA | AYWGQGTTLTVSSASTKGPSVFPLAPSSKSTSGGTA | YSASNRYTGVPDRFTG |
| | | | | PGQGLEWIGN | VAWFQQKP | ALGCLVKDYFPEPVTVSWNSGALTSGVHTFPAVLQS | SGSGTDFTLTIGNMQS |
| | | | | IYPFSSSTNY | GQSPKLLI | SGLYSLSSVVTVPSSSLGTQTYICNVNHKPSNTKVD | EDLADYFCQQYSTDPY |
| | | | | NEKFKKKATL | YSASNRYT | KKVEPKSCDKTHTCPPCPAPELLGGPSVFLFPPKPK | TFGSGTKLEIKRTVAA |
| | | | | TVDASSSTAS | GVPDRFTG | DTLMISRTPEVTCVVVDVSHEDPEVKFNWYVDGVEV | PSVFIFPPSDEQLKSG |
| | | | | MQLSSLTSDD | SGSGTDFT | HNAKTKPREEQYNSTYRVVSVLTVLHQDWLNGKEYK | TASVVCLLNNFYPREA |
| | | | | SAVYFCAREE | LTIGNMQS | CKVSNKALPAPIEKTISKAKGQPREPQVYTLPPSRE | KVQWKVDNALQSGNSQ |
| | | | | FSHYPSWFAY | EDLADYFC | EMTKNQVSLTCLVKGFYPSDIAVEWESNGQPENNYK | ESVTEQDSKDSTYSLS |
| | | | | WGQGTTLTVS | QQYSTDPY | TTPPVLDSDGSFFLYSKLTVDKSRWQQGNVFSCSVM | STLTLSKADYEKHKVY |
| | | | | S | TFGSGTKL | HEALHNHYTQKSLSLSPGK | ACEVTHQGLSSPVTKS |
| | | | | | EIK | | FNRGEC |
| | | | | 1663 | 1664 | 1665 | 1666 |
| 50 | CD8B370 | IgG1 | Kappa | EVQLQQSGAE | DIVLTQSQ | EVQLQQSGAELVKPGASVKLSCKASGYTFTSYWINW | DIVLTQSQKIMSTTVG |
| | | | | LVKPGASVKL | KIMSTTVG | VKQRPGQGLEWIGNIYPGSSSTNYNEKFKNKATLTV | DRVSITCKASQNVGTA |
| | | | | SCKASGYTFT | DRVSITCK | DTSSSTVYMQLSSLTSDDSAVYYCTRELGAYYHYSA | VAWYQQKPGQSPKLLI |
| | | | | SYWINWVKQR | ASQNVGTA | MDYWGQGTSVTVSSASTKGPSVFPLAPSSKSTSGGT | YSASNRYTGVPDRFTG |
| | | | | PGQGLEWIGN | VAWYQQKP | AALGCLVKDYFPEPVTVSWNSGALTSGVHTFPAVLQ | SGSGTDFTLTISNMQS |
| | | | | IYPGSSSTNY | GQSPKLLI | SSGLYSLSSVVTVPSSSLGTQTYICNVNHKPSNTKV | EDLADYFCQQYSIYPF |
| | | | | NEKFKNKATL | YSASNRYT | DKKVEPKSCDKTHTCPPCPAPELLGGPSVFLFPPKP | TFGSGTKLEIKRTVAA |
| | | | | TVDTSSSTVY | GVPDRFTG | KDTLMISRTPEVTCVVVDVSHEDPEVKFNWYVDGVE | PSVFIFPPSDEQLKSG |
| | | | | MQLSSLTSDD | SGSGTDFT | VHNAKTKPREEQYNSTYRVVSVLTVLHQDWLNGKEY | TASVVCLLNNFYPREA |
| | | | | SAVYYCTREL | LTISNMQS | KCKVSNKALPAPIEKTISKAKGQPREPQVYTLPPSR | KVQWKVDNALQSGNSQ |
| | | | | GAYYHYSAMD | EDLADYFC | EEMTKNQVSLTCLVKGFYPSDIAVEWESNGQPENNY | ESVTEQDSKDSTYSLS |
| | | | | YWGQGTSVTV | QQYSIYPF | KTTPPVLDSDGSFFLYSKLTVDKSRWQQGNVFSCSV | STLTLSKADYEKHKVY |
| | | | | SS | TFGSGTKL | MHEALHNHYTQKSLSLSPGK | ACEVTHQGLSSPVTKS |
| | | | | | EIK | | FNRGEC |
| | | | | 1697 | 1698 | 1699 | 1700 |
| 51 | CD8B186 | IgG1 | Kappa | QVQLQQSGAE | DVQMIQSP | QVQLQQSGAELAKPGASVKMSCKASGYIFTSYWMHW | DVQMIQSPASLSASVG |
| | | | | LAKPGASVKM | ASLSASVG | VKQRPGQGLEWIGNINPSSGYAVYNQKFKDKATLTA | ETVTITCRASGNIHNY |
| | | | | SCKASGYIFT | ETVTITCR | DQSSSTAYIQLNSLTSEDSAVYYCARRVFYGDSWFA | LAWYQQKQGKSPQLLV |
| | | | | SYWMHWVKQR | ASGNIHNY | YWGQGTSVTVSSASTKGPSVFPLAPSSKSTSGGTAA | YNAKTLADGVPSRFSG |
| | | | | PGQGLEWIGN | LAWYQQKQ | LGCLVKDYFPEPVTVSWNSGALTSGVHTFPAVLQSS | SGSGTQYSLKINSLQP |
| | | | | INPSSGYAVY | GKSPQLLV | GLYSLSSVVTVPSSSLGTQTYICNVNHKPSNTKVDK | EDFGSYYCQHFWSTTW |
| | | | | NQKFKDKATL | YNAKTLAD | KVEPKSCDKTHTCPPCPAPELLGGPSVFLFPPKPKD | TFGGGTKLEIKRTVAA |
| | | | | TADQSSSTAY | GVPSRFSG | TLMISRTPEVTCVVVDVSHEDPEVKFNWYVDGVEVH | PSVFIFPPSDEQLKSG |
| | | | | IQLNSLTSED | SGSGTQYS | NAKTKPREEQYNSTYRVVSVLTVLHQDWLNGKEYKC | TASVVCLLNNFYPREA |
| | | | | SAVYYCARRV | LKINSLQP | KVSNKALPAPIEKTISKAKGQPREPQVYTLPPSREE | KVQWKVDNALQSGNSQ |
| | | | | FYGDSWFAYW | EDFGSYYC | MTKNQVSLTCLVKGFYPSDIAVEWESNGQPENNYKT | ESVTEQDSKDSTYSLS |
| | | | | GQGTSVTVSS | QHFWSTTW | TPPVLDSDGSFFLYSKLTVDKSRWQQGNVFSCSVMH | STLTLSKADYEKHKVY |
| | | | | | TFGGGTKL | EALHNHYTQKSLSLSPGK | ACEVTHQGLSSPVTKS |
| | | | | | EIK | | FNRGEC |
| | | | | 1731 | 1732 | 1733 | 1734 |
| 52 | CD8B190 | IgG1 | Kappa | EFQLQQSGPE | NTQMNQTP | EFQLQQSGPELMKPGASVKISCKASGYSFTSYYMHW | NTQMNQTPSSLSASLG |
| | | | | LMKPGASVKI | SSLSASLG | MKQSHGKSLEWIGYIDPFNGNTNYKQKFKGKATLTV | DTVTITCHASQNINVW |
| | | | | SCKASGYSFT | DTVTITCH | DKSSSTAYMHLSSLTSEDSAVYYCASPNSNYVGTWF | LSWYQQKPGNIPKLLI |
| | | | | SYYMHWMKQS | ASQNINVW | AYWGQGTTVTVSSASTKGPSVFPLAPSSKSTSGGTA | YKASNLHTGVPSRFSG |
| | | | | HGKSLEWIGY | LSWYQQKP | ALGCLVKDYFPEPVTVSWNSGALTSGVHTFPAVLQS | SGSGTGFTLTISSLQP |
| | | | | IDPFNGNTNY | GNIPKLLI | SGLYSLSSVVTVPSSSLGTQTYICNVNHKPSNTKVD | DDIATYYCQQGQSFPF |
| | | | | KQKFKGKATL | YKASNLHT | KKVEPKSCDKTHTCPPCPAPELLGGPSVFLFPPKPK | TFGSGTKLEIKRTVAA |
| | | | | TVDKSSSTAY | GVPSRFSG | DTLMISRTPEVTCVVVDVSHEDPEVKFNWYVDGVEV | PSVFIFPPSDEQLKSG |
| | | | | MHLSSLTSED | SGSGTGFT | HNAKTKPREEQYNSTYRVVSVLTVLHQDWLNGKEYK | TASVVCLLNNFYPREA |
| | | | | SAVYYCASPN | LTISSLQP | CKVSNKALPAPIEKTISKAKGQPREPQVYTLPPSRE | KVQWKVDNALQSGNSQ |
| | | | | SNYVGTWFAY | DDIATYYC | EMTKNQVSLTCLVKGFYPSDIAVEWESNGQPENNYK | ESVTEQDSKDSTYSLS |
| | | | | WGQGTTVTVS | QQGQSFPF | TTPPVLDSDGSFFLYSKLTVDKSRWQQGNVFSCSVM | STLTLSKADYEKHKVY |
| | | | | S | TFGSGTKL | HEALHNHYTQKSLSLSPGK | ACEVTHQGLSSPVTKS |
| | | | | | EIK | | FNRGEC |
| | | | | 1765 | 1766 | 1767 | 1768 |
| 53 | CD8B192 | IgG1 | Kappa | QVQLQQSGPV | DIQMTQSP | QVQLQQSGPVLVKPGASVKMSCKASGYTFTDYYMNW | DIQMTQSPASLSASVG |
| | | | | LVKPGASVKM | ASLSASVG | VMQSHGKSLEWIGVINPYNGGTTYNQRFTGKATLTV | ETVTITCRASGNIHNY |
| | | | | SCKASGYTFT | ETVTITCR | DKSSSTAYMELNSLTSEDSAVYYCARNYGAMDSWGQ | LAWYQQKQGKSPQLLV |
| | | | | DYYMNWVMQS | ASGNIHNY | GTSVTVSSASTKGPSVFPLAPSSKSTSGGTAALGCL | SNAKTLADGVPSRFGG |
| | | | | HGKSLEWIGV | LAWYQQKQ | VKDYFPEPVTVSWNSGALTSGVHTFPAVLQSSGLYS | SGSGTQYSLKINSLQP |
| | | | | INPYNGGTTY | GKSPQLLV | LSSVVTVPSSSLGTQTYICNVNHKPSNTKVDKKVEP | EDFGSYYCQHFWITPP |
| | | | | NQRFTGKATL | SNAKTLAD | KSCDKTHTCPPCPAPELLGGPSVFLFPPKPKDTLMI | TFGAGTRLEIKRTVAA |
| | | | | TVDKSSSTAY | GVPSRFGG | SRTPEVTCVVVDVSHEDPEVKFNWYVDGVEVHNAKT | PSVFIFPPSDEQLKSG |
| | | | | MELNSLTSED | SGSGTQYS | KPREEQYNSTYRVVSVLTVLHQDWLNGKEYKCKVSN | TASVVCLLNNFYPREA |
| | | | | SAVYYCARNY | LKINSLQP | KALPAPIEKTISKAKGQPREPQVYTLPPSREEMTKN | KVQWKVDNALQSGNSQ |
| | | | | GAMDSWGQGT | EDFGSYYC | QVSLTCLVKGFYPSDIAVEWESNGQPENNYKTTPPV | ESVTEQDSKDSTYSLS |
| | | | | SVTVSS | QHFWITPP | LDSDGSFFLYSKLTVDKSRWQQGNVFSCSVMHEALH | STLTLSKADYEKHKVY |
| | | | | | TFGAGTRL | NHYTQKSLSLSPGK | ACEVTHQGLSSPVTKS |
| | | | | | EIK | | FNRGEC |
| | | | | 1799 | 1800 | 1801 | 1802 |
| 54 | CD8B193 | IgG1 | Kappa | DVQLQESGPE | DIVMTQSQ | DVQLQESGPELVKPGASVKIACKTSGYKFTDYYMNW | DIVMTQSQKFMSTTVG |
| | | | | LVKPGASVKI | KFMSTTVG | VKQSLGKSLDWIGDINPNGGGTSDNPKFKGKATLTV | DRVSITCKASQNVGTA |
| | | | | ACKTSGYKFT | DRVSITCK | DKSSSTAYMELRSLTSEDSGVYYCARTSGTDWYFDV | VAWYQQKPGQSPKLLI |
| | | | | DYYMNWVKQS | ASQNVGTA | WGTGTTVTVSSASTKGPSVFPLAPSSKSTSGGTAAL | YSASNRYTGVPDRFTG |
| | | | | LGKSLDWIGD | VAWYQQKP | GCLVKDYFPEPVTVSWNSGALTSGVHTFPAVLQSSG | SGSGTDFTLTISNMQS |
| | | | | INPNGGGTSD | GQSPKLLI | LYSLSSVVTVPSSSLGTQTYICNVNHKPSNTKVDKK | EDLADYFCQQYSSYPF |
| | | | | NPKFKGKATL | YSASNRYT | VEPKSCDKTHTCPPCPAPELLGGPSVFLFPPKPKDT | TFGSGTKLEMKRTVAA |
| | | | | TVDKSSSTAY | GVPDRFTG | LMISRTPEVTCVVVDVSHEDPEVKFNWYVDGVEVHN | PSVFIFPPSDEQLKSG |
| | | | | MELRSLTSED | SGSGTDFT | AKTKPREEQYNSTYRVVSVLTVLHQDWLNGKEYKCK | TASVVCLLNNFYPREA |
| | | | | SGVYYCARTS | LTISNMQS | VSNKALPAPIEKTISKAKGQPREPQVYTLPPSREEM | KVQWKVDNALQSGNSQ |
| | | | | GTDWYFDVWG | EDLADYFC | TKNQVSLTCLVKGFYPSDIAVEWESNGQPENNYKTT | ESVTEQDSKDSTYSLS |
| | | | | TGTTVTVSS | QQYSSYPF | PPVLDSDGSFFLYSKLTVDKSRWQQGNVFSCSVMHE | STLTLSKADYEKHKVY |
| | | | | | TFGSGTKL | ALHNHYTQKSLSLSPGK | ACEVTHQGLSSPVTKS |
| | | | | | EMK | | FNRGEC |
| | | | | 1833 | 1834 | 1835 | 1836 |
| 55 | CD8B214 | IgG1 | Kappa | QVQLQQSGPE | DIQMTQTT | QVQLQQSGPELKKPGETVKISCKASGYTFTTAGIQW | DIQMTQTTSSLSASLG |
| | | | | LKKPGETVKI | SSLSASLG | VQKMPGKGFKWIGWINTHAGESKYADDFKGRFAVSL | DRVTITCRASQDIRPY |
| | | | | SCKASGYTFT | DRVTITCR | ETSASTAYLQISNLKNEDTATYFCARSGDYDGSHPF | LNWYQQKPEGTIKLLI |
| | | | | TAGIQWVQKM | ASQDIRPY | AYWGQGTSVTVSSASTKGPSVFPLAPSSKSTSGGTA | YYTSRLHSGVPSRFSG |
| | | | | PGKGFKWIGW | LNWYQQKP | ALGCLVKDYFPEPVTVSWNSGALTSGVHTFPAVLQS | SGSGTDYSLTISNLEQ |
| | | | | INTHAGESKY | EGTIKLLI | SGLYSLSSVVTVPSSSLGTQTYICNVNHKPSNTKVD | EDIATYFCQQDNTLPY |
| | | | | ADDFKGRFAV | YYTSRLHS | KKVEPKSCDKTHTCPPCPAPELLGGPSVFLFPPKPK | TFGSGTKLEIKRTVAA |
| | | | | SLETSASTAY | GVPSRFSG | DTLMISRTPEVTCVVVDVSHEDPEVKFNWYVDGVEV | PSVFIFPPSDEQLKSG |
| | | | | LQISNLKNED | SGSGTDYS | HNAKTKPREEQYNSTYRVVSVLTVLHQDWLNGKEYK | TASVVCLLNNFYPREA |
| | | | | TATYFCARSG | LTISNLEQ | CKVSNKALPAPIEKTISKAKGQPREPQVYTLPPSRE | KVQWKVDNALQSGNSQ |
| | | | | DYDGSHPFAY | EDIATYFC | EMTKNQVSLTCLVKGFYPSDIAVEWESNGQPENNYK | ESVTEQDSKDSTYSLS |
| | | | | WGQGTSVTVS | QQDNTLPY | TTPPVLDSDGSFFLYSKLTVDKSRWQQGNVFSCSVM | STLTLSKADYEKHKVY |
| | | | | S | TFGSGTKL | HEALHNHYTQKSLSLSPGK | ACEVTHQGLSSPVTKS |
| | | | | | EIK | | FNRGEC |
| | | | | 1867 | 1868 | 1869 | 1870 |
| 56 | CD8B230 | IgG1 | Kappa | QIQLVQSGPE | DIVMTQSQ | QIQLVQSGPELVKPGASVKISCKASGYTFTDYYMNW | DIVMTQSQKFMSTTVG |
| | | | | LVKPGASVKI | KFMSTTVG | VKQSHGKSLDWIGDINPNGGGTSDNPKFKGKATLTV | DRVSITCKASQNVGTA |
| | | | | SCKASGYTFT | DRVSITCK | DKSSNTAYMELRSLTSEDSAVYYCARTSGTDWYFDV | VAWYQQKPGQSPKLLI |
| | | | | DYYMNWVKQS | ASQNVGTA | WGTGTLVTVSAASTKGPSVFPLAPSSKSTSGGTAAL | YSTSNRYTGVPDRFTG |
| | | | | HGKSLDWIGD | VAWYQQKP | GCLVKDYFPEPVTVSWNSGALTSGVHTFPAVLQSSG | SGSGTDFTLTISNMQS |
| | | | | INPNGGGTSD | GQSPKLLI | LYSLSSVVTVPSSSLGTQTYICNVNHKPSNTKVDKK | EDLADYFCQQYSIYPF |
| | | | | NPKFKGKATL | YSTSNRYT | VEPKSCDKTHTCPPCPAPELLGGPSVFLFPPKPKDT | TFGSGTKLEMKRTVAA |
| | | | | TVDKSSNTAY | GVPDRFTG | LMISRTPEVTCVVVDVSHEDPEVKFNWYVDGVEVHN | PSVFIFPPSDEQLKSG |
| | | | | MELRSLTSED | SGSGTDFT | AKTKPREEQYNSTYRVVSVLTVLHQDWLNGKEYKCK | TASVVCLLNNFYPREA |
| | | | | SAVYYCARTS | LTISNMQS | VSNKALPAPIEKTISKAKGQPREPQVYTLPPSREEM | KVQWKVDNALQSGNSQ |
| | | | | GTDWYFDVWG | EDLADYFC | TKNQVSLTCLVKGFYPSDIAVEWESNGQPENNYKTT | ESVTEQDSKDSTYSLS |
| | | | | TGTLVTVSA | QQYSIYPF | PPVLDSDGSFFLYSKLTVDKSRWQQGNVFSCSVMHE | STLTLSKADYEKHKVY |
| | | | | | TFGSGTKL | ALHNHYTQKSLSLSPGK | ACEVTHQGLSSPVTKS |
| | | | | | EMK | | FNRGEC |
| | | | | 1901 | 1902 | 1903 | 1904 |
| 57 | CD8B245 | IgG1 | Kappa | EFQLQQSGGG | DIQMTQSP | EFQLQQSGGGLVQPGGSLSLSCAAPGFTFTDYYMSW | DIQMTQSPASLSASVG |
| | | | | LVQPGGSLSL | ASLSASVG | VRQSPGKALEWLALSRNKGNGYTTEYSASVKGRFTI | ETVTITCRASENIYSY |
| | | | | SCAAPGFTFT | ETVTITCR | SRDNSQSILYLQMNVLRAEDSATYYCARTVTGTLFY | LAWYQQKQGKSPQFLV |
| | | | | DYYMSWVRQS | ASENIYSY | YALDYWGQGTTVTVSSASTKGPSVFPLAPSSKSTSG | YNAKTLAAGVPSRFSG |
| | | | | PGKALEWLAL | LAWYQQKQ | GTAALGCLVKDYFPEPVTVSWNSGALTSGVHTFPAV | SGSGTQFSLKINRLQP |
| | | | | SRNKGNGYTT | GKSPQFLV | LQSSGLYSLSSVVTVPSSSLGTQTYICNVNHKPSNT | EDFGTYYCQHHYGTPL |
| | | | | EYSASVKGRF | YNAKTLAA | KVDKKVEPKSCDKTHTCPPCPAPELLGGPSVFLFPP | TFGDGTRLEIKRTVAA |
| | | | | TISRDNSQSI | GVPSRFSG | KPKDTLMISRTPEVTCVVVDVSHEDPEVKFNWYVDG | PSVFIFPPSDEQLKSG |
| | | | | LYLQMNVLRA | SGSGTQFS | VEVHNAKTKPREEQYNSTYRVVSVLTVLHQDWLNGK | TASVVCLLNNFYPREA |
| | | | | EDSATYYCAR | LKINRLQP | EYKCKVSNKALPAPIEKTISKAKGQPREPQVYTLPP | KVQWKVDNALQSGNSQ |
| | | | | TVTGTLFYYA | EDFGTYYC | SREEMTKNQVSLTCLVKGFYPSDIAVEWESNGQPEN | ESVTEQDSKDSTYSLS |
| | | | | LDYWGQGTTV | QHHYGTPL | NYKTTPPVLDSDGSFFLYSKLTVDKSRWQQGNVFSC | STLTLSKADYEKHKVY |
| | | | | TVSS | TFGDGTRL | SVMHEALHNHYTQKSLSLSPGK | ACEVTHQGLSSPVTKS |
| | | | | | EIK | | FNRGEC |
| | | | | 1935 | 1936 | 1937 | 1938 |
| 58 | CD8B248 | IgG1 | Kappa | EVQLQQSGAE | DVVMTQTP | EVQLQQSGAELARPGASVKMSCKASGYTFTTYTMHW | DVVMTQTPLSLPVSLG |
| | | | | LARPGASVKM | LSLPVSLG | VKQRPGQGLEWIGYINPSSGYTKYNQKFTDKATLTA | DQASISCRSSQSLVHS |
| | | | | SCKASGYTFT | DQASISCR | DKSSSTAYMQLSSLTSEDSAVYYCARLWAYWGQGTL | SGNTYLHWYLQKPGQS |
| | | | | TYTMHWVKQR | SSQSLVHS | VTVSAASTKGPSVFPLAPSSKSTSGGTAALGCLVKD | PKLLIYKGSNRFSGVS |
| | | | | PGQGLEWIGY | SGNTYLHW | YFPEPVTVSWNSGALTSGVHTFPAVLQSSGLYSLSS | DRFSGSGSGTDFTLKI |
| | | | | INPSSGYTKY | YLQKPGQS | VVTVPSSSLGTQTYICNVNHKPSNTKVDKKVEPKSC | SRVEAEDLGVYFCSQS |
| | | | | NQKFTDKATL | PKLLIYKG | DKTHTCPPCPAPELLGGPSVFLFPPKPKDTLMISRT | THVPFTFGSGTKLEMK |
| | | | | TADKSSSTAY | SNRFSGVS | PEVTCVVVDVSHEDPEVKFNWYVDGVEVHNAKTKPR | RTVAAPSVFIFPPSDE |
| | | | | MQLSSLTSED | DRFSGSGS | EEQYNSTYRVVSVLTVLHQDWLNGKEYKCKVSNKAL | QLKSGTASVVCLLNNF |
| | | | | SAVYYCARLW | GTDFTLKI | PAPIEKTISKAKGQPREPQVYTLPPSREEMTKNQVS | YPREAKVQWKVDNALQ |
| | | | | AYWGQGTLVT | SRVEAEDL | LTCLVKGFYPSDIAVEWESNGQPENNYKTTPPVLDS | SGNSQESVTEQDSKDS |
| | | | | VSA | GVYFCSQS | DGSFFLYSKLTVDKSRWQQGNVFSCSVMHEALHNHY | TYSLSSTLTLSKADYE |
| | | | | | THVPFTFG | TQKSLSLSPGK | KHKVYACEVTHQGLSS |
| | | | | | SGTKLEMK | | PVTKSFNRGEC |
| | | | | 1969 | 1970 | 1971 | 1972 |
| 59 | CD8B250 | IgG1 | Kappa | QVQLKESGPG | DIVMTQSQ | QVQLKESGPGLVAPSQSLSITCTVSGFSLSNYVVHW | DIVMTQSQKFMSTSVG |
| | | | | LVAPSQSLSI | KFMSTSVG | VRQSPGKGLEWLGVIWTDGSTDYNAAFISRLSISKD | DRVSVTCKASQNVDTD |
| | | | | TCTVSGFSLS | DRVSVTCK | NSKSQVFFKMNSLQADDTAIYYCARNNGYFPAFFAY | ITWYQQKPGQSPKALI |
| | | | | NYVVHWVRQS | ASQNVDTD | WGQGTLVTVSAASTKGPSVFPLAPSSKSTSGGTAAL | YSASYRYSGVPDRFTG |
| | | | | PGKGLEWLGV | ITWYQQKP | GCLVKDYFPEPVTVSWNSGALTSGVHTFPAVLQSSG | SGSGTDFTLTITNVQS |
| | | | | IWTDGSTDYN | GQSPKALI | LYSLSSVVTVPSSSLGTQTYICNVNHKPSNTKVDKK | EDLAEYFCQQYNSYPL |
| | | | | AAFISRLSIS | YSASYRYS | VEPKSCDKTHTCPPCPAPELLGGPSVFLFPPKPKDT | TFGSGTKLEMKRTVAA |
| | | | | KDNSKSQVFF | GVPDRFTG | LMISRTPEVTCVVVDVSHEDPEVKFNWYVDGVEVHN | PSVFIFPPSDEQLKSG |
| | | | | KMNSLQADDT | SGSGTDFT | AKTKPREEQYNSTYRVVSVLTVLHQDWLNGKEYKCK | TASVVCLLNNFYPREA |
| | | | | AIYYCARNNG | LTITNVQS | VSNKALPAPIEKTISKAKGQPREPQVYTLPPSREEM | KVQWKVDNALQSGNSQ |
| | | | | YFPAFFAYWG | EDLAEYFC | TKNQVSLTCLVKGFYPSDIAVEWESNGQPENNYKTT | ESVTEQDSKDSTYSLS |
| | | | | QGTLVTVSA | QQYNSYPL | PPVLDSDGSFFLYSKLTVDKSRWQQGNVFSCSVMHE | STLTLSKADYEKHKVY |
| | | | | | TFGSGTKL | ALHNHYTQKSLSLSPGK | ACEVTHQGLSSPVTKS |
| | | | | | EMK | | FNRGEC |
| | | | | 2003 | 2004 | 2005 | 2006 |
| 60 | CD8B254 | IgG1 | Kappa | EVQLQQSGAE | DVVMTQTP | EVQLQQSGAELVKPGASVKMSCKTSGYTFSSYWITW | DVVMTQTPLSLPVSLG |
| | | | | LVKPGASVKM | LSLPVSLG | VKQRPGQGLEWVGDIYPGSGSTNYNEKFKSKAALTV | DQASISCRSSQSLVHS |
| | | | | SCKTSGYTFS | DQASISCR | DTSSSTAFMQLNSLTSEDSAVYYCARESITTRITPF | SGNTYLHWYLQKPGQS |
| | | | | SYWITWVKQR | SSQSLVHS | DHWGQGTTLTVSSASTKGPSVFPLAPSSKSTSGGTA | PKLLIYKGSNRFSGVS |
| | | | | PGQGLEWVGD | SGNTYLHW | ALGCLVKDYFPEPVTVSWNSGALTSGVHTFPAVLQS | DRFSGSGSGTDFTLKI |
| | | | | IYPGSGSTNY | YLQKPGQS | SGLYSLSSVVTVPSSSLGTQTYICNVNHKPSNTKVD | SRVEAEDLGVYFCSQS |
| | | | | NEKFKSKAAL | PKLLIYKG | KKVEPKSCDKTHTCPPCPAPELLGGPSVFLFPPKPK | THVPFTFGSGTKLEIK |
| | | | | TVDTSSSTAF | SNRFSGVS | DTLMISRTPEVTCVVVDVSHEDPEVKFNWYVDGVEV | RTVAAPSVFIFPPSDE |
| | | | | MQLNSLTSED | DRFSGSGS | HNAKTKPREEQYNSTYRVVSVLTVLHQDWLNGKEYK | QLKSGTASVVCLLNNF |
| | | | | SAVYYCARES | GTDFTLKI | CKVSNKALPAPIEKTISKAKGQPREPQVYTLPPSRE | YPREAKVQWKVDNALQ |
| | | | | ITTRITPFDH | SRVEAEDL | EMTKNQVSLTCLVKGFYPSDIAVEWESNGQPENNYK | SGNSQESVTEQDSKDS |
| | | | | WGQGTTLTVS | GVYFCSQS | TTPPVLDSDGSFFLYSKLTVDKSRWQQGNVFSCSVM | TYSLSSTLTLSKADYE |
| | | | | S | THVPFTFG | HEALHNHYTQKSLSLSPGK | KHKVYACEVTHQGLSS |
| | | | | | SGTKLEIK | | PVTKSFNRGEC |
| | | | | 2037 | 2038 | 2039 | 2040 |
| 61 | CD8B261 | IgG1 | Kappa | QVQLQQPGAE | DIVLTQSP | QVQLQQPGAELVKPGASVKLSCKASGYTFNSYWINW | DIVLTQSPSSMYASLG |
| | | | | LVKPGASVKL | SSMYASLG | MKQRPGQGLEWIGNIYPGSSSTNYNEKFKSKATLTV | ERVTITCKASQDINRY |
| | | | | SCKASGYTFN | ERVTITCK | DTSSSTAYMQLSSLTSDDSAVYYCARELGGYYRYNA | LSWFQQKPGKSPKTLI |
| | | | | SYWINWMKQR | ASQDINRY | MDYWGQGTSVTVSSASTKGPSVFPLAPSSKSTSGGT | YRANTLVDGVPSRFSG |
| | | | | PGQGLEWIGN | LSWFQQKP | AALGCLVKDYFPEPVTVSWNSGALTSGVHTFPAVLQ | SGSGQDYSLTISSLEY |
| | | | | IYPGSSSTNY | GKSPKTLI | SSGLYSLSSVVTVPSSSLGTQTYICNVNHKPSNTKV | EDMGIYYCLQYDEFPY |
| | | | | NEKFKSKATL | YRANTLVD | DKKVEPKSCDKTHTCPPCPAPELLGGPSVFLFPPKP | TFGSGTKLEMKRTVAA |
| | | | | TVDTSSSTAY | GVPSRFSG | KDTLMISRTPEVTCVVVDVSHEDPEVKFNWYVDGVE | PSVFIFPPSDEQLKSG |
| | | | | MQLSSLTSDD | SGSGQDYS | VHNAKTKPREEQYNSTYRVVSVLTVLHQDWLNGKEY | TASVVCLLNNFYPREA |
| | | | | SAVYYCAREL | LTISSLEY | KCKVSNKALPAPIEKTISKAKGQPREPQVYTLPPSR | KVQWKVDNALQSGNSQ |
| | | | | GGYYRYNAMD | EDMGIYYC | EEMTKNQVSLTCLVKGFYPSDIAVEWESNGQPENNY | ESVTEQDSKDSTYSLS |
| | | | | YWGQGTSVTV | LQYDEFPY | KTTPPVLDSDGSFFLYSKLTVDKSRWQQGNVFSCSV | STLTLSKADYEKHKVY |
| | | | | SS | TFGSGTKL | MHEALHNHYTQKSLSLSPGK | ACEVTHQGLSSPVTKS |
| | | | | | EMK | | FNRGEC |
| | | | | 2071 | 2072 | 2073 | 2074 |
| 62 | CD8B311 | IgG1 | Kappa | QVQLKESGPE | DIQMTQTT | QVQLKESGPELVKPGASVKLSCKASGYTFTSYWMHW | DIQMTQTTSSLSASLG |
| | | | | LVKPGASVKL | SSLSASLG | VKQRPGQGLEWIGMIHPNSGSTNYNEKFKSKATLTV | DRVTISCSASQGISNC |
| | | | | SCKASGYTFT | DRVTISCS | DKSSSTAYMQLSSLTSEDSAVYYCARCGYDGAWFAY | LNWYQQKPDGTVKLLI |
| | | | | SYWMHWVKQR | ASQGISNC | WGQGTSVTVSSASTKGPSVFPLAPSSKSTSGGTAAL | HYTSSLHSGVPSRFSG |
| | | | | PGQGLEWIGM | LNWYQQKP | GCLVKDYFPEPVTVSWNSGALTSGVHTFPAVLQSSG | GGSGTHYSLTISNLEP |
| | | | | IHPNSGSTNY | DGTVKLLI | LYSLSSVVTVPSSSLGTQTYICNVNHKPSNTKVDKK | EDIATYYCQQYSKVPY |
| | | | | NEKFKSKATL | HYTSSLHS | VEPKSCDKTHTCPPCPAPELLGGPSVFLFPPKPKDT | TFGSGTKLEIKRTVAA |
| | | | | TVDKSSSTAY | GVPSRFSG | LMISRTPEVTCVVVDVSHEDPEVKFNWYVDGVEVHN | PSVFIFPPSDEQLKSG |
| | | | | MQLSSLTSED | GGSGTHYS | AKTKPREEQYNSTYRVVSVLTVLHQDWLNGKEYKCK | TASVVCLLNNFYPREA |
| | | | | SAVYYCARCG | LTISNLEP | VSNKALPAPIEKTISKAKGQPREPQVYTLPPSREEM | KVQWKVDNALQSGNSQ |
| | | | | YDGAWFAYWG | EDIATYYC | TKNQVSLTCLVKGFYPSDIAVEWESNGQPENNYKTT | ESVTEQDSKDSTYSLS |
| | | | | QGTSVTVSS | QQYSKVPY | PPVLDSDGSFFLYSKLTVDKSRWQQGNVFSCSVMHE | STLTLSKADYEKHKVY |
| | | | | | TFGSGTKL | ALHNHYTQKSLSLSPGK | ACEVTHQGLSSPVTKS |
| | | | | | EIK | | FNRGEC |
| | | | | 2105 | 2106 | 2107 | 2108 |
| 63 | CD8B340 | IgG1 | Kappa | QVQLQQPGAE | DIVMTQTP | QVQLQQPGAELVKPGASVRLSCKASGYTFTNYWMQW | DIVMTQTPLTLSVTIG |
| | | | | LVKPGASVRL | LTLSVTIG | VQQRPGQGLEWIGEIDPSDTFTNYNQNFKDKATLTV | QPASISCKSSQSLLYS |
| | | | | SCKASGYTFT | QPASISCK | DTSSSTAYLQLSSLTSEDSAVYYCARGDWDRDWYFD | DGKTYLNWLLQRPGES |
| | | | | NYWMQWVQQR | SSQSLLYS | VWGTGTLVTVSAASTKGPSVFPLAPSSKSTSGGTAA | PKLLIYLVSKLDSGVP |
| | | | | PGQGLEWIGE | DGKTYLNW | LGCLVKDYFPEPVTVSWNSGALTSGVHTFPAVLQSS | DRFTGSGSGTDFTLKI |
| | | | | IDPSDTFTNY | LLQRPGES | GLYSLSSVVTVPSSSLGTQTYICNVNHKPSNTKVDK | SRVETEDLGIYYCLQA |
| | | | | NQNFKDKATL | PKLLIYLV | KVEPKSCDKTHTCPPCPAPELLGGPSVFLFPPKPKD | THFPHTFGAGTKLELK |
| | | | | TVDTSSSTAY | SKLDSGVP | TLMISRTPEVTCVVVDVSHEDPEVKFNWYVDGVEVH | RTVAAPSVFIFPPSDE |
| | | | | LQLSSLTSED | DRFTGSGS | NAKTKPREEQYNSTYRVVSVLTVLHQDWLNGKEYKC | QLKSGTASVVCLLNNF |
| | | | | SAVYYCARGD | GTDFTLKI | KVSNKALPAPIEKTISKAKGQPREPQVYTLPPSREE | YPREAKVQWKVDNALQ |
| | | | | WDRDWYFDVW | SRVETEDL | MTKNQVSLTCLVKGFYPSDIAVEWESNGQPENNYKT | SGNSQESVTEQDSKDS |
| | | | | GTGTLVTVSA | GIYYCLQA | TPPVLDSDGSFFLYSKLTVDKSRWQQGNVFSCSVMH | TYSLSSTLTLSKADYE |
| | | | | | THFPHTFG | EALHNHYTQKSLSLSPGK | KHKVYACEVTHQGLSS |
| | | | | | AGTKLELK | | PVTKSFNRGEC |
| | | | | 2139 | 2140 | 2141 | 2142 |
| 64 | CD8B362 | IgG1 | Kappa | EVKLVESGAE | DIQMTQSP | EVKLVESGAELVKPGASVKLSCTASGFNIKDTYMHW | DIQMTQSPSSLSASLG |
| | | | | LVKPGASVKL | SSLSASLG | VKQRPEQGLEWIGRIDPANGHTKFDPKFQGKATITA | DRVSLTCRASHEISGY |
| | | | | SCTASGFNIK | DRVSLTCR | DTSSNTAYLQLSSLTSEDTAVYYCAIRFAYWGQGTL | LSWLQQKPDGTFKRLI |
| | | | | DTYMHWVKQR | ASHEISGY | VTVSAASTKGPSVFPLAPSSKSTSGGTAALGCLVKD | YAASTLDSGVPKRFSG |
| | | | | PEQGLEWIGR | LSWLQQKP | YFPEPVTVSWNSGALTSGVHTFPAVLQSSGLYSLSS | SRSGSDYSLSISSLES |
| | | | | IDPANGHTKF | DGTFKRLI | VVTVPSSSLGTQTYICNVNHKPSNTKVDKKVEPKSC | EDFADYYCLQYSSYPY |
| | | | | DPKFQGKATI | YAASTLDS | DKTHTCPPCPAPELLGGPSVFLFPPKPKDTLMISRT | TFGSGTKLEMKRTVAA |
| | | | | TADTSSNTAY | GVPKRFSG | PEVTCVVVDVSHEDPEVKFNWYVDGVEVHNAKTKPR | PSVFIFPPSDEQLKSG |
| | | | | LQLSSLTSED | SRSGSDYS | EEQYNSTYRVVSVLTVLHQDWLNGKEYKCKVSNKAL | TASVVCLLNNFYPREA |
| | | | | TAVYYCAIRF | LSISSLES | PAPIEKTISKAKGQPREPQVYTLPPSREEMTKNQVS | KVQWKVDNALQSGNSQ |
| | | | | AYWGQGTLVT | EDFADYYC | LTCLVKGFYPSDIAVEWESNGQPENNYKTTPPVLDS | ESVTEQDSKDSTYSLS |
| | | | | VSA | LQYSSYPY | DGSFFLYSKLTVDKSRWQQGNVFSCSVMHEALHNHY | STLTLSKADYEKHKVY |
| | | | | | TFGSGTKL | TQKSLSLSPGK | ACEVTHQGLSSPVTKS |
| | | | | | EMK | | FNRGEC |
| | | | | 2173 | 2174 | 2175 | 2176 |

TABLE 17
Kabat CDR Amino Acid Sequences

| | Protein | HC Kabat | | | | LC Kabat | LC Kabat |
|---|---|---|---|---|---|---|---|
| # | Name | CDR1 | HC Kabat CDR2 | HC Kabat CDR3 | LC Kabat CDR1 | CDR2 | CDR3 |
| 1 | CD8B191 | DYYMN | RVIPSNGGTIYNLKFKG | EDYNNQGFFLDAMDY | RASQSISDFLH | YASQSIS | QNGHSFPYT |
| | | 1 | 2 | 3 | 4 | 5 | 6 |
| 2 | CD8B226 | DYYMN | RIIPSNGATIYNQKFKG | EDYSNQGFFLDAMDY | RASQSISHYLH | YASQSIS | QNGHSFPYT |
| | | 35 | 36 | 37 | 38 | 39 | 40 |
| 3 | CD8B259 | DYYMN | RVIPSNGGTIYNQKFRG | EDYGNQGFFLDAMDY | RASQSISHFLH | YASQSIS | QSGHSFPYT |
| | | 69 | 70 | 71 | 72 | 73 | 74 |
| 4 | CD8B298 | DYYMN | RVIPNNGGTRYNQKFKG | EDFSNQGFFLDAMDY | RASQTISDYLH | YASQSIS | QNGHSFPYT |
| | | 103 | 104 | 105 | 106 | 107 | 108 |
| 5 | CD8B342 | DYYVN | RVIPNNGNVIYNQNFKG | EDYSNQGFFLDAMDY | RASQTISNYLH | YASQSIS | QNGHSFPYT |
| | | 137 | 138 | 139 | 140 | 141 | 142 |
| 6 | CD8B364 | SYWMH | EINPSNGDSYYNEKFKR | SMYYDGRAGAY | ITSTDIDDDMN | EGNTLRP | LQSDNMPLT |
| | | 171 | 172 | 173 | 174 | 175 | 176 |
| 7 | CD8B200 | NYWIH | NIDPSDSETHYNQKFKD | GLTGTGYY | RASQDISPYLN | YTSKLHS | QQDNTLPYT |
| | | 205 | 206 | 207 | 208 | 209 | 210 |
| 8 | CD8B247 | DYYMN | RVIPNNGGTIYNQKFKD | EDYSNQGFFLDAMDY | RASQTISHFLH | YASQSIS | QSGHSFPYT |
| | | 239 | 240 | 241 | 242 | 243 | 244 |
| 9 | CD8B265 | DYYMN | RVIPRNGATTYNQNFRG | EDFSNQGFFLDAMDY | RASQSISHYLH | YASQSIS | QNGHSFPYT |
| | | 273 | 274 | 275 | 276 | 277 | 278 |
| 10 | CD8B270 | NYWMH | NIDPSDSETHYNQKFKD | GLTGTGYY | RASQDIRPYLN | FTSKLHS | QQDNTLPYT |
| | | 307 | 308 | 309 | 310 | 311 | 312 |
| 11 | CD8B213 | DYYMD | YIYPNNGITSYNQKFKG | SIYYDHGGGFPY | KASQNVDKYVA | SASYRYS | QQYNTYPS |
| | | 341 | 342 | 343 | 344 | 345 | 346 |
| 12 | CD8B240 | DYYMN | RVIPSNGGTIYNLKFKG | EDYNNQGFFLDAMDY | RASQSISDFLH | YASQSIS | QNGHSFPYT |
| | | 375 | 376 | 377 | 378 | 379 | 380 |
| 13 | CD8B361 | DYYMD | YIYPNNGDTRYNQKFKD | SIYYDHGGGFPY | KASQNVGTYVA | SASYRYS | QQYNSYPT |
| | | 409 | 410 | 411 | 412 | 413 | 414 |
| 14 | CD8B246 | TSGMNVG | HIWWDDDKYYNPSLKS | RGNYGNYEFAY | RASQDIRNYLN | HTSRLHS | QQGNTLPWT |
| | | 443 | 444 | 445 | 446 | 447 | 448 |
| 15 | CD8B268 | VYTIH | WFYPGSGNIKYNEKFKD | HEDNHYYDGNSWFAY | RASGNIHNYLA | NAKTLAD | QHFWNTPYT |
| | | 477 | 478 | 479 | 480 | 481 | 482 |
| 16 | CD8B271 | IYSIH | MIWGGGDTDYNSALKS | NPHYYGGTYEYFDV | SASQGISNYLN | DTSILYS | QQYSNLPYT |
| | | 511 | 512 | 513 | 514 | 515 | 516 |
| 17 | CD8B273 | EYTIH | WFYPGTGSIKYNEKFKD | HEDNHYYDGNSWFAY | RASGNIHNYLA | NAKTLAD | QHFWSTPYT |
| | | 545 | 546 | 547 | 548 | 549 | 550 |
| 18 | CD8B288 | EYTIH | WFYPGNGNMRYNEKFKD | YEDNHYYDGASWFAY | RASGNIHNYLA | NAKTLAD | QHFWSTPFT |
| | | 579 | 580 | 581 | 582 | 583 | 584 |
| 19 | CD8B292 | DDYIY | WIDPENGATEYASKFQG | HDYGYAMDY | TASSSVSSSYLH | STSNLAS | HQYHRSPLT |
| | | 613 | 614 | 615 | 616 | 617 | 618 |
| 20 | CD8B303 | IYSIH | MIWGGGSTDYNSTLNS | NPHHYGGSTGAMDY | KASQDIKKYMA | YTSSLQP | LQYDNLFT |
| | | 647 | 648 | 649 | 650 | 651 | 652 |
| 21 | CD8B304 | TSGMNVG | HIWWDDDKYYNPSLKS | RGNYGNYEFAY | RASQDIRNYLN | HTSRLHS | QQGNTLPWT |
| | | 681 | 682 | 683 | 684 | 685 | 686 |
| 22 | CD8B312 | SFWMH | NVDPSDSQTHYNQKFKD | STYYRYDGPFTY | RASQSINNNLH | YTSQSIS | QQSNSWPLT |
| | | 715 | 716 | 717 | 718 | 719 | 720 |
| 23 | CD8B347 | SYWMN | AVNPSNSYTEYAQKFKD | SGLYNTNHLAWFAY | RASGNIHNYLA | NAETLAD | QHFWNNPLT |
| | | 749 | 750 | 751 | 752 | 753 | 754 |
| 24 | CD8B350 | AYWIN | SINPSNGYTEYSQKFKD | SGLYYTNHLAWCPY | RASGNIHNYLA | NAETLAD | QHFWNSPLT |
| | | 783 | 784 | 785 | 786 | 787 | 788 |
| 25 | CD8B356 | SGYYWN | YISYDGSNNYNPSLKN | NHGDAMDY | KASQNVGTAVA | SASYRYT | QQYSSYLT |
| | | 817 | 818 | 819 | 820 | 821 | 822 |
| 26 | CD8B369 | NTYIS | WIYTGTGGTWYNQKFTD | TNWDWYFDV | RASENIYSYLA | YAKTLTD | QHHYGRPYT |
| | | 851 | 852 | 853 | 854 | 855 | 856 |
| 27 | CD8B371 | DYYMA | HINYDGSITYYLDSLKS | EDYSNYGFAY | HASQNINVWLS | KASNLHT | QQGQSYPLT |
| | | 885 | 886 | 887 | 888 | 889 | 890 |
| 28 | CD8B182 | SYWMN | AVNPTNYYTEYIQKFKD | SGLYNTNHLAWFAY | RASENIHNYLA | NAKTLAN | QHFWTTPLT |
| | | 919 | 920 | 921 | 922 | 923 | 924 |
| 29 | CD8B205 | SYWMH | NIDPSDSETHYNQKFKD | VYYSYYSYDATYFDY | RASENIYSYLA | NAKTLAE | QHHYTTPLT |
| | | 953 | 954 | 955 | 956 | 957 | 958 |
| 30 | CD8B223 | SYSVH | VIWAGGSTNYNSAFMS | HSYYSFDAFDY | KASQNVNTDVA | SASYRYS | QQCNSYPLT |
| | | 987 | 988 | 989 | 990 | 991 | 992 |
| 31 | CD8B234 | SGYYWN | YINYDGRNNYNPSLKN | DQGYSKFYFDY | KASEDIYNRLA | GATSLET | QQYWSFPRT |
| | | 1021 | 1022 | 1023 | 1024 | 1025 | 1026 |
| 32 | CD8B251 | TYAVH | VIWSGGSTDYNAAFIS | HSYYHYNAMDN | KASQNVGTAVA | SASNRYT | QQYSSYPFT |
| | | 1055 | 1056 | 1057 | 1058 | 1059 | 1060 |
| 33 | CD8B269 | SGYYWN | YISYDGSNNYNPSLKN | NHGDAMDH | KASQNVGTDVA | SASYRYS | QQYKSYPLT |
| | | 1089 | 1090 | 1091 | 1092 | 1093 | 1094 |
| 34 | CD8B290 | RYSVH | MIWGGGSTDYNSALKS | IYFDNYVGFAY | KASQDVGTVVA | WTSTRHT | QQYSSYPYT |
| | | 1123 | 1124 | 1125 | 1126 | 1127 | 1128 |
| 35 | CD8B310 | NYAVH | VIWTDGSTDYNAGFIS | NNGYFPAFFAY | RSSQTIVHSNGNTYLE | KVSNRFS | FQGSHAPFT |
| | | 1157 | 1158 | 1159 | 1160 | 1161 | 1162 |
| 36 | CD8B352 | SGYYWN | YINYDGRNNYNPSLRN | DQGYSKFYFDY | KASEDIYNRLA | GATSLET | QQYWSFPRT |
| | | 1191 | 1192 | 1193 | 1194 | 1195 | 1196 |
| 37 | CD8B319 | AYYMH | EINPSAGGTTYNQKFKA | WTNPFDY | KASQNVGTAVA | SASYRYT | QQYNNYLT |
| | | 1225 | 1226 | 1227 | 1228 | 1229 | 1230 |
| 38 | CD8B194 | SYWIN | NIYPGSSSTNYNEKFKS | ELGPYYRYSAMVY | KASQNVGTAVA | SASNRYT | QQYSSYPFT |
| | | 1259 | 1260 | 1261 | 1262 | 1263 | 1264 |
| 39 | CD8B231 | NYWMH | NIDPSDSETHYNQKFKD | GLTGTGHY | RASQDINIYLN | HTSRLHS | QQDNTLPYT |
| | | 1293 | 1294 | 1295 | 1296 | 1297 | 1298 |
| 40 | CD8B238 | DYSMD | YIYTYSGGAGYNRKFKS | DSSDYEFAY | KASQDIKSYLS | RANRLVD | LQYDEFRT |
| | | 1327 | 1328 | 1329 | 1330 | 1331 | 1332 |
| 41 | CD8B255 | TSGMGVS | HIFWDDDKRYNPSLKS | RDGYGDYAYFDV | RASENIYSDLA | AATILTD | QHFWGTPWT |
| | | 1361 | 1362 | 1363 | 1364 | 1365 | 1366 |
| 42 | CD8B324 | SHWIH | NIYPGSSSTNYNEKFKR | HSPGHRDYAMDY | KASQNVGTAVA | SASNRYT | QQYSTYPLT |
| | | 1395 | 1396 | 1397 | 1398 | 1399 | 1400 |
| 43 | CD8B337 | TSGMGVS | HIFWDDDRRYKSSLKS | RVGYGDYAYFDV | RASENIYSDLA | AATNLAD | QHFWGTPWT |
| | | 1429 | 1430 | 1431 | 1432 | 1433 | 1434 |
| 44 | CD8B344 | NYWIN | NIYPGSDSSNYNEKFKT | EEADYRYTWFVY | KASQNVGTAVA | SASNRYT | QQYSSYPLT |
| | | 1463 | 1464 | 1465 | 1466 | 1467 | 1468 |
| 45 | CD8B264 | SYWIN | NIYPGSSSTNYNEKFKN | EEYSYKSSWFAY | KASQNVGTAVA | SASNRYN | QQYSTYPYT |
| | | 1497 | 1498 | 1499 | 1500 | 1501 | 1502 |
| 46 | CD8B318 | SYWIS | NIYPGSSSSNYNENFKS | EEYSYFPSWFAY | KASQNVGTAVA | SASNRYT | QQYSTYPFT |
| | | 1531 | 1532 | 1533 | 1534 | 1535 | 1536 |
| 47 | CD8B333 | SFWIN | NIYPGSSSTNYSEKFKN | EEYSYKSSWFAY | KASQNVGTAVA | SASNRYN | QQYSTYPYT |
| | | 1565 | 1566 | 1567 | 1568 | 1569 | 1570 |
| 48 | CD8B366 | DDYIH | RIDPANGNPRYAPKFQD | DDEGYYYFDV | RASKSISKYLA | SGSTLQS | QQHNEYPLT |
| | | 1599 | 1600 | 1601 | 1602 | 1603 | 1604 |
| 49 | CD8B368 | SYWIN | NIYPFSSSTNYNEKFKK | EEFSHYPSWFAY | KASQNVGIAVA | SASNRYT | QQYSTDPYT |
| | | 1633 | 1634 | 1635 | 1636 | 1637 | 1638 |
| 50 | CD8B370 | SYWIN | NIYPGSSSTNYNEKFKN | ELGAYYHYSAMDY | KASQNVGTAVA | SASNRYT | QQYSIYPFT |
| | | 1667 | 1668 | 1669 | 1670 | 1671 | 1672 |
| 51 | CD8B186 | SYWMH | NINPSSGYAVYNQKFKD | RVFYGDSWFAY | RASGNIHNYLA | NAKTLAD | QHFWSTTWT |
| | | 1701 | 1702 | 1703 | 1704 | 1705 | 1706 |
| 52 | CD8B190 | SYYMH | YIDPFNGNTNYKQKFKG | PNSNYVGTWFAY | HASQNINVWLS | KASNLHT | QQGQSFPFT |
| | | 1735 | 1736 | 1737 | 1738 | 1739 | 1740 |
| 53 | CD8B192 | DYYMN | VINPYNGGTTYNQRFTG | NYGAMDS | RASGNIHNYLA | NAKTLAD | QHFWITPPT |
| | | 1769 | 1770 | 1771 | 1772 | 1773 | 1774 |
| 54 | CD8B193 | DYYMN | DINPNGGGTSDNPKFKG | TSGTDWYFDV | KASQNVGTAVA | SASNRYT | QQYSSYPFT |
| | | 1803 | 1804 | 1805 | 1806 | 1807 | 1808 |
| 55 | CD8B214 | TAGIQ | WINTHAGESKYADDFKG | SGDYDGSHPFAY | RASQDIRPYLN | YTSRLHS | QQDNTLPYT |
| | | 1837 | 1838 | 1839 | 1840 | 1841 | 1842 |
| 56 | CD8B230 | DYYMN | DINPNGGGTSDNPKFKG | TSGTDWYFDV | KASQNVGTAVA | STSNRYT | QQYSIYPFT |
| | | 1871 | 1872 | 1873 | 1874 | 1875 | 1876 |
| 57 | CD8B245 | DYYMS | LSRNKGNGYTTEYSASVKG | TVTGTLFYYALDY | RASENIYSYLA | NAKTLAA | QHHYGTPLT |
| | | 1905 | 1906 | 1907 | 1908 | 1909 | 1910 |
| 58 | CD8B248 | TYTMH | YINPSSGYTKYNQKFTD | LWAY | RSSQSLVHSSGNTYLH | KGSNRFS | SQSTHVPFT |
| | | 1939 | 1940 | 1941 | 1942 | 1943 | 1944 |
| 59 | CD8B250 | NYVVH | VIWTDGSTDYNAAFIS | NNGYFPAFFAY | KASQNVDTDIT | SASYRYS | QQYNSYPLT |
| | | 1973 | 1974 | 1975 | 1976 | 1977 | 1978 |
| 60 | CD8B254 | SYWIT | DIYPGSGSTNYNEKFKS | ESITTRITPFDH | RSSQSLVHSSGNTYLH | KGSNRFS | SQSTHVPFT |
| | | 2007 | 2008 | 2009 | 2010 | 2011 | 2012 |
| 61 | CD8B261 | SYWIN | NIYPGSSSTNYNEKFKS | ELGGYYRYNAMDY | KASQDINRYLS | RANTLVD | LQYDEFPYT |
| | | 2041 | 2042 | 2043 | 2044 | 2045 | 2046 |
| 62 | CD8B311 | SYWMH | MIHPNSGSTNYNEKFKS | CGYDGAWFAY | SASQGISNCLN | YTSSLHS | QQYSKVPYT |
| | | 2075 | 2076 | 2077 | 2078 | 2079 | 2080 |
| 63 | CD8B340 | NYWMQ | EIDPSDTFTNYNQNFKD | GDWDRDWYFDV | KSSQSLLYSDGKTYLN | LVSKLDS | LQATHFPHT |
| | | 2109 | 2110 | 2111 | 2112 | 2113 | 2114 |
| 64 | CD8B362 | DTYMH | RIDPANGHTKFDPKFQG | RFAY | RASHEISGYLS | AASTLDS | LQYSSYPYT |
| | | 2143 | 2144 | 2145 | 2146 | 2147 | 2148 |

TABLE 18
Chothia CDR Amino Acid Sequences

| | Protein | HC Chothia | HC Chothia | | | LC Chothia | LC Chothia |
|---|---|---|---|---|---|---|---|
| # | Name | CDR1 | CDR2 | HC Chothia CDR3 | LC Chothia CDR1 | CDR2 | CDR3 |
| 1 | CD8B191 | GYTFTDY | IPSNGG | EDYNNQGFFLDAMD | SQSISDF | YAS | GHSFPY |
| | | 7 | 8 | 9 | 10 | 11 | 12 |
| 2 | CD8B226 | GYTFTDY | IPSNGA | EDYSNQGFFLDAMD | SQSISHY | YAS | GHSFPY |
| | | 41 | 42 | 43 | 44 | 45 | 46 |
| 3 | CD8B259 | GYTFTDY | IPSNGG | EDYGNQGFFLDAMD | SQSISHF | YAS | GHSFPY |
| | | 75 | 76 | 77 | 78 | 79 | 80 |
| 4 | CD8B298 | GYTFTDY | IPNNGG | EDFSNQGFFLDAMD | SQTISDY | YAS | GHSFPY |
| | | 109 | 110 | 111 | 112 | 113 | 114 |
| 5 | CD8B342 | GYTFTDY | IPNNGN | EDYSNQGFFLDAMD | SQTISNY | YAS | GHSFPY |
| | | 143 | 144 | 145 | 146 | 147 | 148 |
| 6 | CD8B364 | GYTFTSY | NPSNGD | SMYYDGRAGA | STDIDDD | EGN | SDNMPL |
| | | 177 | 178 | 179 | 180 | 181 | 182 |
| 7 | CD8B200 | GYTFTNY | DPSDSE | GLTGTGY | SQDISPY | YTS | DNTLPY |
| | | 211 | 212 | 213 | 214 | 215 | 216 |
| 8 | CD8B247 | GYTFTDY | IPNNGG | EDYSNQGFFLDAMD | SQTISHF | YAS | GHSFPY |
| | | 245 | 246 | 247 | 248 | 249 | 250 |
| 9 | CD8B265 | GYSFTDY | IPRNGA | EDFSNQGFFLDAMD | SQSISHY | YAS | GHSFPY |
| | | 279 | 280 | 281 | 282 | 283 | 284 |
| 10 | CD8B270 | GYTFTNY | DPSDSE | GLTGTGY | SQDIRPY | FTS | DNTLPY |
| | | 313 | 314 | 315 | 316 | 317 | 318 |
| 11 | CD8B213 | GYIFTDY | YPNNGI | SIYYDHGGGFP | SQNVDKY | SAS | YNTYP |
| | | 347 | 348 | 349 | 350 | 351 | 352 |
| 12 | CD8B240 | GYTFTDY | IPSNGG | EDYNNQGFFLDAMD | SQSISDF | YAS | GHSFPY |
| | | 381 | 382 | 383 | 384 | 385 | 386 |
| 13 | CD8B361 | GYTFTDY | YPNNGD | SIYYDHGGGFP | SQNVGTY | SAS | YNSYP |
| | | 415 | 416 | 417 | 418 | 419 | 420 |
| 14 | CD8B246 | GFSLSTSGM | WWDDD | RGNYGNYEFA | SQDIRNY | HTS | GNTLPW |
| | | 449 | 450 | 451 | 452 | 453 | 454 |
| 15 | CD8B268 | GYTFTVY | YPGSGN | HEDNHYYDGNSWFA | SGNIHNY | NAK | FWNTPY |
| | | 483 | 484 | 485 | 486 | 487 | 488 |
| 16 | CD8B271 | GFSLSIY | WGGGD | NPHYYGGTYEYFD | SQGISNY | DTS | YSNLPY |
| | | 517 | 518 | 519 | 520 | 521 | 522 |
| 17 | CD8B273 | GYTFTEY | YPGTGS | HEDNHYYDGNSWFA | SGNIHNY | NAK | FWSTPY |
| | | 551 | 552 | 553 | 554 | 555 | 556 |
| 18 | CD8B288 | GYTFTEY | YPGNGN | YEDNHYYDGASWFA | SGNIHNY | NAK | FWSTPF |
| | | 585 | 586 | 587 | 588 | 589 | 590 |
| 19 | CD8B292 | GFNFKDD | DPENGA | HDYGYAMD | SSSVSSSY | STS | YHRSPL |
| | | 619 | 620 | 621 | 622 | 623 | 624 |
| 20 | CD8B303 | GFSLSIY | WGGGS | NPHHYGGSTGAMD | SQDIKKY | YTS | YDNLF |
| | | 653 | 654 | 655 | 656 | 657 | 658 |
| 21 | CD8B304 | GFSLSTSGM | WWDDD | RGNYGNYEFA | SQDIRNY | HTS | GNTLPW |
| | | 687 | 688 | 689 | 690 | 691 | 692 |
| 22 | CD8B312 | GYTFTSF | DPSDSQ | STYYRYDGPFT | SQSINNN | YTS | SNSWPL |
| | | 721 | 722 | 723 | 724 | 725 | 726 |
| 23 | CD8B347 | GYTFTSY | NPSNSY | SGLYNTNHLAWFA | SGNIHNY | NAE | FWNNPL |
| | | 755 | 756 | 757 | 758 | 759 | 760 |
| 24 | CD8B350 | GYTFAAY | NPSNGY | SGLYYTNHLAWCP | SGNIHNY | NAE | FWNSPL |
| | | 789 | 790 | 791 | 792 | 793 | 794 |
| 25 | CD8B356 | GYSITSGY | SYDGS | NHGDAMD | SQNVGTA | SAS | YSSYL |
| | | 823 | 824 | 825 | 826 | 827 | 828 |
| 26 | CD8B369 | GFTFTNT | YTGTGG | TNWDWYFD | SENIYSY | YAK | HYGRPY |
| | | 857 | 858 | 859 | 860 | 861 | 862 |
| 27 | CD8B371 | GFTFSDY | NYDGSI | EDYSNYGFA | SQNINVW | KAS | GQSYPL |
| | | 891 | 892 | 893 | 894 | 895 | 896 |
| 28 | CD8B182 | GYTFTSY | NPTNYY | SGLYNTNHLAWFA | SENIHNY | NAK | FWTTPL |
| | | 925 | 926 | 927 | 928 | 929 | 930 |
| 29 | CD8B205 | GYSFNSY | DPSDSE | VYYSYYSYDATYFD | SENIYSY | NAK | HYTTPL |
| | | 959 | 960 | 961 | 962 | 963 | 964 |
| 30 | CD8B223 | GFSLTSY | WAGGS | HSYYSFDAFD | SQNVNTD | SAS | CNSYPL |
| | | 993 | 994 | 995 | 996 | 997 | 998 |
| 31 | CD8B234 | GYSITSGY | NYDGR | DQGYSKFYFD | SEDIYNR | GAT | YWSFPR |
| | | 1027 | 1028 | 1029 | 1030 | 1031 | 1032 |
| 32 | CD8B251 | GFSLTTY | WSGGS | HSYYHYNAMD | SQNVGTA | SAS | YSSYPF |
| | | 1061 | 1062 | 1063 | 1064 | 1065 | 1066 |
| 33 | CD8B269 | GYSITSGY | SYDGS | NHGDAMD | SQNVGTD | SAS | YKSYPL |
| | | 1095 | 1096 | 1097 | 1098 | 1099 | 1100 |
| 34 | CD8B290 | GFSLSRY | WGGGS | IYFDNYVGFA | SQDVGTV | WTS | YSSYPY |
| | | 1129 | 1130 | 1131 | 1132 | 1133 | 1134 |
| 35 | CD8B310 | GFSLTNY | WTDGS | NNGYFPAFFA | SQTIVHSNGNTY | KVS | GSHAPF |
| | | 1163 | 1164 | 1165 | 1166 | 1167 | 1168 |
| 36 | CD8B352 | GYSITSGY | NYDGR | DQGYSKFYFD | SEDIYNR | GAT | YWSFPR |
| | | 1197 | 1198 | 1199 | 1200 | 1201 | 1202 |
| 37 | CD8B319 | GYSFTAY | NPSAGG | WTNPFD | SQNVGTA | SAS | YNNYL |
| | | 1231 | 1232 | 1233 | 1234 | 1235 | 1236 |
| 38 | CD8B194 | GYTFTSY | YPGSSS | ELGPYYRYSAMV | SQNVGTA | SAS | YSSYPF |
| | | 1265 | 1266 | 1267 | 1268 | 1269 | 1270 |
| 39 | CD8B231 | GYTFTNY | DPSDSE | GLTGTGH | SQDINIY | HTS | DNTLPY |
| | | 1299 | 1300 | 1301 | 1302 | 1303 | 1304 |
| 40 | CD8B238 | GYTFTDY | YTYSGG | DSSDYEFA | SQDIKSY | RAN | YDEFR |
| | | 1333 | 1334 | 1335 | 1336 | 1337 | 1338 |
| 41 | CD8B255 | GFSLNTSGM | FWDDD | RDGYGDYAYFD | SENIYSD | AAT | FWGTPW |
| | | 1367 | 1368 | 1369 | 1370 | 1371 | 1372 |
| 42 | CD8B324 | GYTSTSH | YPGSSS | HSPGHRDYAMD | SQNVGTA | SAS | YSTYPL |
| | | 1401 | 1402 | 1403 | 1404 | 1405 | 1406 |
| 43 | CD8B337 | GFSLSTSGM | FWDDD | RVGYGDYAYFD | SENIYSD | AAT | FWGTPW |
| | | 1435 | 1436 | 1437 | 1438 | 1439 | 1440 |
| 44 | CD8B344 | GYSFTNY | YPGSDS | EEADYRYTWFV | SQNVGTA | SAS | YSSYPL |
| | | 1469 | 1470 | 1471 | 1472 | 1473 | 1474 |
| 45 | CD8B264 | GYSFTSY | YPGSSS | EEYSYKSSWFA | SQNVGTA | SAS | YSTYPY |
| | | 1503 | 1504 | 1505 | 1506 | 1507 | 1508 |
| 46 | CD8B318 | GYTFTSY | YPGSSS | EEYSYFPSWFA | SQNVGTA | SAS | YSTYPF |
| | | 1537 | 1538 | 1539 | 1540 | 1541 | 1542 |
| 47 | CD8B333 | GYSFASF | YPGSSS | EEYSYKSSWFA | SQNVGTA | SAS | YSTYPY |
| | | 1571 | 1572 | 1573 | 1574 | 1575 | 1576 |
| 48 | CD8B366 | GFNIKDD | DPANGN | DDEGYYYFD | SKSISKY | SGS | HNEYPL |
| | | 1605 | 1606 | 1607 | 1608 | 1609 | 1610 |
| 49 | CD8B368 | GYTFTSY | YPFSSS | EEFSHYPSWFA | SQNVGIA | SAS | YSTDPY |
| | | 1639 | 1640 | 1641 | 1642 | 1643 | 1644 |
| 50 | CD8B370 | GYTFTSY | YPGSSS | ELGAYYHYSAMD | SQNVGTA | SAS | YSIYPF |
| | | 1673 | 1674 | 1675 | 1676 | 1677 | 1678 |
| 51 | CD8B186 | GYIFTSY | NPSSGY | RVFYGDSWFA | SGNIHNY | NAK | FWSTTW |
| | | 1707 | 1708 | 1709 | 1710 | 1711 | 1712 |
| 52 | CD8B190 | GYSFTSY | DPFNGN | PNSNYVGTWFA | SQNINVW | KAS | GQSFPF |
| | | 1741 | 1742 | 1743 | 1744 | 1745 | 1746 |
| 53 | CD8B192 | GYTFTDY | NPYNGG | NYGAMD | SGNIHNY | NAK | FWITPP |
| | | 1775 | 1776 | 1777 | 1778 | 1779 | 1780 |
| 54 | CD8B193 | GYKFTDY | NPNGGG | TSGTDWYFD | SQNVGTA | SAS | YSSYPF |
| | | 1809 | 1810 | 1811 | 1812 | 1813 | 1814 |
| 55 | CD8B214 | GYTFTTA | NTHAGE | SGDYDGSHPFA | SQDIRPY | YTS | DNTLPY |
| | | 1843 | 1844 | 1845 | 1846 | 1847 | 1848 |
| 56 | CD8B230 | GYTFTDY | NPNGGG | TSGTDWYFD | SQNVGTA | STS | YSIYPF |
| | | 1877 | 1878 | 1879 | 1880 | 1881 | 1882 |
| 57 | CD8B245 | GFTFTDY | RNKGNGYT | TVTGTLFYYALD | SENIYSY | NAK | HYGTPL |
| | | 1911 | 1912 | 1913 | 1914 | 1915 | 1916 |
| 58 | CD8B248 | GYTFTTY | NPSSGY | LWA | SQSLVHSSGNTY | KGS | STHVPF |
| | | 1945 | 1946 | 1947 | 1948 | 1949 | 1950 |
| 59 | CD8B250 | GFSLSNY | WTDGS | NNGYFPAFFA | SQNVDTD | SAS | YNSYPL |
| | | 1979 | 1980 | 1981 | 1982 | 1983 | 1984 |
| 60 | CD8B254 | GYTFSSY | YPGSGS | ESITTRITPFD | SQSLVHSSGNTY | KGS | STHVPF |
| | | 2013 | 2014 | 2015 | 2016 | 2017 | 2018 |
| 61 | CD8B261 | GYTFNSY | YPGSSS | ELGGYYRYNAMD | SQDINRY | RAN | YDEFPY |
| | | 2047 | 2048 | 2049 | 2050 | 2051 | 2052 |
| 62 | CD8B311 | GYTFTSY | HPNSGS | CGYDGAWFA | SQGISNC | YTS | YSKVPY |
| | | 2081 | 2082 | 2083 | 2084 | 2085 | 2086 |
| 63 | CD8B340 | GYTFTNY | DPSDTF | GDWDRDWYFD | SQSLLYSDGKTY | LVS | ATHFPH |
| | | 2115 | 2116 | 2117 | 2118 | 2119 | 2120 |
| 64 | CD8B362 | GFNIKDT | DPANGH | RFA | SHEISGY | AAS | YSSYPY |
| | | 2149 | 2150 | 2151 | 2152 | 2153 | 2154 |

TABLE 19
AbM CDR Amino Acid Sequences

| | Protein | | | | | LC AbM | |
|---|---|---|---|---|---|---|---|
| # | Name | HC AbM CDR1 | HC AbM CDR2 | HC AbM CDR3 | LC AbM CDR1 | CDR2 | LC AbM CDR3 |
| 1 | CD8B191 | GYTFTDYYMN | RVIPSNGGTI | EDYNNQGFFLDAMDY | RASQSISDFLH | YASQSIS | QNGHSFPYT |
| | | 13 | 14 | 15 | 16 | 17 | 18 |
| 2 | CD8B226 | GYTFTDYYMN | RIIPSNGATI | EDYSNQGFFLDAMDY | RASQSISHYLH | YASQSIS | QNGHSFPYT |
| | | 47 | 48 | 49 | 50 | 51 | 52 |
| 3 | CD8B259 | GYTFTDYYMN | RVIPSNGGTI | EDYGNQGFFLDAMDY | RASQSISHFLH | YASQSIS | QSGHSFPYT |
| | | 81 | 82 | 83 | 84 | 85 | 86 |
| 4 | CD8B298 | GYTFTDYYMN | RVIPNNGGTR | EDFSNQGFFLDAMDY | RASQTISDYLH | YASQSIS | QNGHSFPYT |
| | | 115 | 116 | 117 | 118 | 119 | 120 |
| 5 | CD8B342 | GYTFTDYYVN | RVIPNNGNVI | EDYSNQGFFLDAMDY | RASQTISNYLH | YASQSIS | QNGHSFPYT |
| | | 149 | 150 | 151 | 152 | 153 | 154 |
| 6 | CD8B364 | GYTFTSYWMH | EINPSNGDSY | SMYYDGRAGAY | ITSTDIDDDMN | EGNTLRP | LQSDNMPLT |
| | | 183 | 184 | 185 | 186 | 187 | 188 |
| 7 | CD8B200 | GYTFTNYWIH | NIDPSDSETH | GLTGTGYY | RASQDISPYLN | YTSKLHS | QQDNTLPYT |
| | | 217 | 218 | 219 | 220 | 221 | 222 |
| 8 | CD8B247 | GYTFTDYYMN | RVIPNNGGTI | EDYSNQGFFLDAMDY | RASQTISHFLH | YASQSIS | QSGHSFPYT |
| | | 251 | 252 | 253 | 254 | 255 | 256 |
| 9 | CD8B265 | GYSFTDYYMN | RVIPRNGATT | EDFSNQGFFLDAMDY | RASQSISHYLH | YASQSIS | QNGHSFPYT |
| | | 285 | 286 | 287 | 288 | 289 | 290 |
| 10 | CD8B270 | GYTFTNYWMH | NIDPSDSETH | GLTGTGYY | RASQDIRPYLN | FTSKLHS | QQDNTLPYT |
| | | 319 | 320 | 321 | 322 | 323 | 324 |
| 11 | CD8B213 | GYIFTDYYMD | YIYPNNGITS | SIYYDHGGGFPY | KASQNVDKYVA | SASYRYS | QQYNTYPS |
| | | 353 | 354 | 355 | 356 | 357 | 358 |
| 12 | CD8B240 | GYTFTDYYMN | RVIPSNGGTI | EDYNNQGFFLDAMDY | RASQSISDFLH | YASQSIS | QNGHSFPYT |
| | | 387 | 388 | 389 | 390 | 391 | 392 |
| 13 | CD8B361 | GYTFTDYYMD | YIYPNNGDTR | SIYYDHGGGFPY | KASQNVGTYVA | SASYRYS | QQYNSYPT |
| | | 421 | 422 | 423 | 424 | 425 | 426 |
| 14 | CD8B246 | GFSLSTSGMNVG | HIWWDDDKY | RGNYGNYEFAY | RASQDIRNYLN | HTSRLHS | QQGNTLPWT |
| | | 455 | 456 | 457 | 458 | 459 | 460 |
| 15 | CD8B268 | GYTFTVYTIH | WFYPGSGNIK | HEDNHYYDGNSWFAY | RASGNIHNYLA | NAKTLAD | QHFWNTPYT |
| | | 489 | 490 | 491 | 492 | 493 | 494 |
| 16 | CD8B271 | GFSLSIYSIH | MIWGGGDTD | NPHYYGGTYEYFDV | SASQGISNYLN | DTSILYS | QQYSNLPYT |
| | | 523 | 524 | 525 | 526 | 527 | 528 |
| 17 | CD8B273 | GYTFTEYTIH | WFYPGTGSIK | HEDNHYYDGNSWFAY | RASGNIHNYLA | NAKTLAD | QHFWSTPYT |
| | | 557 | 558 | 559 | 560 | 561 | 562 |
| 18 | CD8B288 | GYTFTEYTIH | WFYPGNGNMR | YEDNHYYDGASWFAY | RASGNIHNYLA | NAKTLAD | QHFWSTPFT |
| | | 591 | 592 | 593 | 594 | 595 | 596 |
| 19 | CD8B292 | GFNFKDDYIY | WIDPENGATE | HDYGYAMDY | TASSSVSSSYLH | STSNLAS | HQYHRSPLT |
| | | 625 | 626 | 627 | 628 | 629 | 630 |
| 20 | CD8B303 | GFSLSTYSTH | MIWGGGSTD | NPHHYGGSTGAMDY | KASQDIKKYMA | YTSSLQP | LQYDNLFT |
| | | 659 | 660 | 661 | 662 | 663 | 664 |
| 21 | CD8B304 | GFSLSTSGMNVG | HIWWDDDKY | RGNYGNYEFAY | RASQDIRNYLN | HTSRLHS | QQGNTLPWT |
| | | 693 | 694 | 695 | 696 | 697 | 698 |
| 22 | CD8B312 | GYTFTSFWMH | NVDPSDSQTH | STYYRYDGPFTY | RASQSINNNLH | YTSQSIS | QQSNSWPLT |
| | | 727 | 728 | 729 | 730 | 731 | 732 |
| 23 | CD8B347 | GYTFTSYWMN | AVNPSNSYTE | SGLYNTNHLAWFAY | RASGNIHNYLA | NAETLAD | QHFWNNPLT |
| | | 761 | 762 | 763 | 764 | 765 | 766 |
| 24 | CD8B350 | GYTFAAYWIN | SINPSNGYTE | SGLYYTNHLAWCPY | RASGNIHNYLA | NAETLAD | QHFWNSPLT |
| | | 795 | 796 | 797 | 798 | 799 | 800 |
| 25 | CD8B356 | GYSITSGYYWNYI | SYDGSNN | NHGDAMDY | KASQNVGTAVA | SASYRYT | QQYSSYLT |
| | | 829 | 830 | 831 | 832 | 833 | 834 |
| 26 | CD8B369 | GFTFTNTYIS | WIYTGTGGTW | TNWDWYFDV | RASENIYSYLA | YAKTLTD | QHHYGRPYT |
| | | 863 | 864 | 865 | 866 | 867 | 868 |
| 27 | CD8B371 | GFTFSDYYMA | HINYDGSITY | EDYSNYGFAY | HASQNINVWLS | KASNLHT | QQGQSYPLT |
| | | 897 | 898 | 899 | 900 | 901 | 902 |
| 28 | CD8B182 | GYTFTSYWMN | AVNPTNYYTE | SGLYNTNHLAWFAY | RASENIHNYLA | NAKTLAN | QHFWTTPLT |
| | | 931 | 932 | 933 | 934 | 935 | 936 |
| 29 | CD8B205 | GYSFNSYWMH | NIDPSDSETH | VYYSYYSYDATYFDY | RASENIYSYLA | NAKTLAE | QHHYTTPLT |
| | | 965 | 966 | 967 | 968 | 969 | 970 |
| 30 | CD8B223 | GFSLTSYSVH | VIWAGGSTN | HSYYSFDAFDY | KASQNVNTDVA | SASYRYS | QQCNSYPLT |
| | | 999 | 1000 | 1001 | 1002 | 1003 | 1004 |
| 31 | CD8B234 | GYSITSGYYWN | YINYDGRNN | DQGYSKFYFDY | KASEDIYNRLA | GATSLET | QQYWSFPRT |
| | | 1033 | 1034 | 1035 | 1036 | 1037 | 1038 |
| 32 | CD8B251 | GFSLTTYAVH | VIWSGGSTD | HSYYHYNAMDN | KASQNVGTAVA | SASNRYT | QQYSSYPFT |
| | | 1067 | 1068 | 1069 | 1070 | 1071 | 1072 |
| 33 | CD8B269 | GYSITSGYYWN | YISYDGSNN | NHGDAMDH | KASQNVGTDVA | SASYRYS | QQYKSYPLT |
| | | 1101 | 1102 | 1103 | 1104 | 1105 | 1106 |
| 34 | CD8B290 | GFSLSRYSVH | MIWGGGSTD | IYFDNYVGFAY | KASQDVGTVVA | WTSTRHT | QQYSSYPYT |
| | | 1135 | 1136 | 1137 | 1138 | 1139 | 1140 |
| 35 | CD8B310 | GFSLTNYAVH | VIWTDGSTD | NNGYFPAFFAY | RSSQTIVHSNGNTYLE | KVSNRFS | FQGSHAPFT |
| | | 1169 | 1170 | 1171 | 1172 | 1173 | 1174 |
| 36 | CD8B352 | GYSITSGYYWN | YINYDGRNN | DQGYSKFYFDY | KASEDIYNRLA | GATSLET | QQYWSFPRT |
| | | 1203 | 1204 | 1205 | 1206 | 1207 | 1208 |
| 37 | CD8B319 | GYSFTAYYMH | EINPSAGGTT | WTNPFDY | KASQNVGTAVA | SASYRYT | QQYNNYLT |
| | | 1237 | 1238 | 1239 | 1240 | 1241 | 1242 |
| 38 | CD8B194 | GYTFTSYWIN | NIYPGSSSTN | ELGPYYRYSAMVY | KASQNVGTAVA | SASNRYT | QQYSSYPFT |
| | | 1271 | 1272 | 1273 | 1274 | 1275 | 1276 |
| 39 | CD8B231 | GYTFTNYWMH | NIDPSDSETH | GLTGTGHY | RASQDINIYLN | HTSRLHS | QQDNTLPYT |
| | | 1305 | 1306 | 1307 | 1308 | 1309 | 1310 |
| 40 | CD8B238 | GYTFTDYSMD | YIYTYSGGAG | DSSDYEFAY | KASQDIKSYLS | RANRLVD | LQYDEFRT |
| | | 1339 | 1340 | 1341 | 1342 | 1343 | 1344 |
| 41 | CD8B255 | GFSLNTSGMGVS | HIFWDDDKR | RDGYGDYAYFDV | RASENIYSDLA | AATILTD | QHFWGTPWT |
| | | 1373 | 1374 | 1375 | 1376 | 1377 | 1378 |
| 42 | CD8B324 | GYTSTSHWIH | NIYPGSSSTN | HSPGHRDYAMDY | KASQNVGTAVA | SASNRYT | QQYSTYPLT |
| | | 1407 | 1408 | 1409 | 1410 | 1411 | 1412 |
| 43 | CD8B337 | GFSLSTSGMGVS | HIFWDDDRR | RVGYGDYAYFDV | RASENIYSDLA | AATNLAD | QHFWGTPWT |
| | | 1441 | 1442 | 1443 | 1444 | 1445 | 1446 |
| 44 | CD8B344 | GYSFTNYWIN | NIYPGSDSSN | EEADYRYTWFVY | KASQNVGTAVA | SASNRYT | QQYSSYPLT |
| | | 1475 | 1476 | 1477 | 1478 | 1479 | 1480 |
| 45 | CD8B264 | GYSFTSYWIN | NIYPGSSSTN | EEYSYKSSWFAY | KASQNVGTAVA | SASNRYN | QQYSTYPYT |
| | | 1509 | 1510 | 1511 | 1512 | 1513 | 1514 |
| 46 | CD8B318 | GYTFTSYWIS | NIYPGSSSSN | EEYSYFPSWFAY | KASQNVGTAVA | SASNRYT | QQYSTYPFT |
| | | 1543 | 1544 | 1545 | 1546 | 1547 | 1548 |
| 47 | CD8B333 | GYSFASFWIN | NIYPGSSSTN | EEYSYKSSWFAY | KASQNVGTAVA | SASNRYN | QQYSTYPYT |
| | | 1577 | 1578 | 1579 | 1580 | 1581 | 1582 |
| 48 | CD8B366 | GFNIKDDYIH | RIDPANGNPR | DDEGYYYFDV | RASKSISKYLA | SGSTLQS | QQHNEYPLT |
| | | 1611 | 1612 | 1613 | 1614 | 1615 | 1616 |
| 49 | CD8B368 | GYTFTSYWIN | NIYPFSSSTN | EEFSHYPSWFAY | KASQNVGIAVA | SASNRYT | QQYSTDPYT |
| | | 1645 | 1646 | 1647 | 1648 | 1649 | 1650 |
| 50 | CD8B370 | GYTFTSYWIN | NIYPGSSSTN | ELGAYYHYSAMDY | KASQNVGTAVA | SASNRYT | QQYSIYPFT |
| | | 1679 | 1680 | 1681 | 1682 | 1683 | 1684 |
| 51 | CD8B186 | GYIFTSYWMH | NINPSSGYAV | RVFYGDSWFAY | RASGNIHNYLA | NAKTLAD | QHFWSTTWT |
| | | 1713 | 1714 | 1715 | 1716 | 1717 | 1718 |
| 52 | CD8B190 | GYSFTSYYMH | YIDPFNGNTN | PNSNYVGTWFAY | HASQNINVWLS | KASNLHT | QQGQSFPFT |
| | | 1747 | 1748 | 1749 | 1750 | 1751 | 1752 |
| 53 | CD8B192 | GYTFTDYYMN | VINPYNGGTT | NYGAMDS | RASGNIHNYLA | NAKTLAD | QHFWITPPT |
| | | 1781 | 1782 | 1783 | 1784 | 1785 | 1786 |
| 54 | CD8B193 | GYKFTDYYMN | DINPNGGGTS | TSGTDWYFDV | KASQNVGTAVA | SASNRYT | QQYSSYPFT |
| | | 1815 | 1816 | 1817 | 1818 | 1819 | 1820 |
| 55 | CD8B214 | GYTFTTAGIQ | WINTHAGESK | SGDYDGSHPFAY | RASQDIRPYLN | YTSRLHS | QQDNTLPYT |
| | | 1849 | 1850 | 1851 | 1852 | 1853 | 1854 |
| 56 | CD8B230 | GYTFTDYYMN | DINPNGGGTS | TSGTDWYFDV | KASQNVGTAVA | STSNRYT | QQYSIYPFT |
| | | 1883 | 1884 | 1885 | 1886 | 1887 | 1888 |
| 57 | CD8B245 | GFTFTDYYMS | LSRNKGNGYTTE | TVTGTLFYYALDY | RASENIYSYLA | NAKTLAA | QHHYGTPLT |
| | | 1917 | 1918 | 1919 | 1920 | 1921 | 1922 |
| 58 | CD8B248 | GYTFTTYTMH | YINPSSGYTK | LWAY | RSSQSLVHSSGNTYLH | KGSNRFS | SQSTHVPFT |
| | | 1951 | 1952 | 1953 | 1954 | 1955 | 1956 |
| 59 | CD8B250 | GFSLSNYVVH | VIWTDGSTD | NNGYFPAFFAY | KASQNVDTDIT | SASYRYS | QQYNSYPLT |
| | | 1985 | 1986 | 1987 | 1988 | 1989 | 1990 |
| 60 | CD8B254 | GYTFSSYWIT | DIYPGSGSTN | ESITTRITPFDH | RSSQSLVHSSGNTYLH | KGSNRFS | SQSTHVPFT |
| | | 2019 | 2020 | 2021 | 2022 | 2023 | 2024 |
| 61 | CD8B261 | GYTFNSYWIN | NIYPGSSSTN | ELGGYYRYNAMDY | KASQDINRYLS | RANTLVD | LQYDEFPYT |
| | | 2053 | 2054 | 2055 | 2056 | 2057 | 2058 |
| 62 | CD8B311 | GYTFTSYWMH | MIHPNSGSTN | CGYDGAWFAY | SASQGISNCLN | YTSSLHS | QQYSKVPYT |
| | | 2087 | 2088 | 2089 | 2090 | 2091 | 2092 |
| 63 | CD8B340 | GYTFTNYWMQ | EIDPSDTFTN | GDWDRDWYFDV | KSSQSLLYSDGKTYLN | LVSKLDS | LQATHFPHT |
| | | 2121 | 2122 | 2123 | 2124 | 2125 | 2126 |
| 64 | CD8B362 | GFNIKDTYMH | RIDPANGHTK | RFAY | RASHEISGYLS | AASTLDS | LQYSSYPYT |
| | | 2155 | 2156 | 2157 | 2158 | 2159 | 2160 |

TABLE 20
Contact CDR Amino Acid Sequences

| | Protein | HC Contact | | | LC Contact | | |
|---|---|---|---|---|---|---|---|
| # | Name | CDR1 | HC Contact CDR2 | HC Contact CDR3 | CDR1 | LC Contact CDR2 | LC Contact CDR3 |
| 1 | CD8B191 | TDYYMN | WIGRVIPSNGGTI | AREDYNNQGFFLDAMD | SDFLHWY | LLIKYASQSI | QNGHSFPY |
| | | 19 | 20 | 21 | 22 | 23 | 24 |
| 2 | CD8B226 | TDYYMN | WIGRIIPSNGATI | AREDYSNQGFFLDAMD | SHYLHWY | LLIKYASQSI | QNGHSFPY |
| | | 53 | 54 | 55 | 56 | 57 | 58 |
| 3 | CD8B259 | TDYYMN | WIGRVIPSNGGTI | AREDYGNQGFFLDAMD | SHFLHWY | LLIKYASQSI | QSGHSFPY |
| | | 87 | 88 | 89 | 90 | 91 | 92 |
| 4 | CD8B298 | TDYYMN | WIGRVIPNNGGTR | AREDFSNQGFFLDAMD | SDYLHWY | LLIKYASQSI | QNGHSFPY |
| | | 121 | 122 | 123 | 124 | 125 | 126 |
| 5 | CD8B342 | TDYYVN | WIGRVIPNNGNVI | TREDYSNQGFFLDAMD | SNYLHWY | LLIKYASQSI | QNGHSFPY |
| | | 155 | 156 | 157 | 158 | 159 | 160 |
| 6 | CD8B364 | TSYWMH | WIGEINPSNGDSY | TRSMYYDGRAGA | DDDMNWY | LLISEGNTLR | LQSDNMPL |
| | | 189 | 190 | 191 | 192 | 193 | 194 |
| 7 | CD8B200 | TNYWIH | WIGNIDPSDSETH | ASGLTGTGY | SPYLNWY | LLIYYTSKLH | QQDNTLPY |
| | | 223 | 224 | 225 | 226 | 227 | 228 |
| 8 | CD8B247 | TDYYMN | WIGRVIPNNGGTI | AREDYSNQGFFLDAMD | SHFLHWY | LLIKYASQSI | QSGHSFPY |
| | | 257 | 258 | 259 | 260 | 261 | 262 |
| 9 | CD8B265 | TDYYMN | WIGRVIPRNGATT | AREDFSNQGFFLDAMD | SHYLHWY | LLIKYASQSI | QNGHSFPY |
| | | 291 | 292 | 293 | 294 | 295 | 296 |
| 10 | CD8B270 | TNYWMH | WIGNIDPSDSETH | ASGLTGTGY | RPYLNWY | LLIYFTSKLH | QQDNTLPY |
| | | 325 | 326 | 327 | 328 | 329 | 330 |
| 11 | CD8B213 | TDYYMD | WIGYIYPNNGITS | ARSIYYDHGGGFP | DKYVAWY | ALIYSASYRY | QQYNTYP |
| | | 359 | 360 | 361 | 362 | 363 | 364 |
| 12 | CD8B240 | TDYYMN | WIGRVIPSNGGTI | AREDYNNQGFFLDAMD | SDFLHWY | LLIKYASQSI | QNGHSFPY |
| | | 393 | 394 | 395 | 396 | 397 | 398 |
| 13 | CD8B361 | TDYYMD | WIGYIYPNNGDTR | ARSIYYDHGGGFP | GTYVAWY | ALIYSASYRY | QQYNSYP |
| | | 427 | 428 | 429 | 430 | 431 | 432 |
| 14 | CD8B246 | STSGMNVG | WLAHIWWDDDKY | ARRGNYGNYEFA | RNYLNWY | LLIYHTSRLH | QQGNTLPW |
| | | 461 | 462 | 463 | 464 | 465 | 466 |
| 15 | CD8B268 | TVYTIH | WIGWFYPGSGNIK | ARHEDNHYYDGNSWFA | HNYLAWF | LLVYNAKTLA | QHFWNTPY |
| | | 495 | 496 | 497 | 498 | 499 | 500 |
| 16 | CD8B271 | SIYSIH | WLGMIWGGGDTD | ARNPHYYGGTYEYFD | SNYLNWY | LLIYDTSILY | QQYSNLPY |
| | | 529 | 530 | 531 | 532 | 533 | 534 |
| 17 | CD8B273 | TEYTIH | WIGWFYPGTGSIK | ARHEDNHYYDGNSWFA | HNYLAWF | LLVYNAKTLA | QHFWSTPY |
| | | 563 | 564 | 565 | 566 | 567 | 568 |
| 18 | CD8B288 | TEYTIH | WIGWFYPGNGNMR | ARYEDNHYYDGASWFA | HNYLAWF | LLVYNAKTLA | QHFWSTPF |
| | | 597 | 598 | 599 | 600 | 601 | 602 |
| 19 | CD8B292 | KDDYIY | WIGWIDPENGATE | SLHDYGYAMD | SSSYLHWY | LWIYSTSNLA | HQYHRSPL |
| | | 631 | 632 | 633 | 634 | 635 | 636 |
| 20 | CD8B303 | STYSTH | WLGMIWGGGSTD | ARNPHHYGGSTGAMD | KKYMAWY | LLIHYTSSLQ | LQYDNLF |
| | | 665 | 666 | 667 | 668 | 669 | 670 |
| 21 | CD8B304 | STSGMNVG | WLAHIWWDDDKY | ARRGNYGNYEFA | RNYLNWY | LLIYHTSRLH | QQGNTLPW |
| | | 699 | 700 | 701 | 702 | 703 | 704 |
| 22 | CD8B312 | TSFWMH | WIGNVDPSDSQTH | ARSTYYRYDGPFT | NNNLHWY | LLIKYTSQSI | QQSNSWPL |
| | | 733 | 734 | 735 | 736 | 737 | 738 |
| 23 | CD8B347 | TSYWMN | WIGAVNPSNSYTE | ARSGLYNTNHLAWFA | HNYLAWY | LLVFNAETLA | QHFWNNPL |
| | | 767 | 768 | 769 | 770 | 771 | 772 |
| 24 | CD8B350 | AAYWIN | WIGSINPSNGYTE | SRSGLYYTNHLAWCP | HNYLAWY | VLVYNAETLA | QHFWNSPL |
| | | 801 | 802 | 803 | 804 | 805 | 806 |
| 25 | CD8B356 | TSGYYWN | WMGYISYDGSNN | VRNHGDAMD | GTAVAWY | LLIYSASYRY | QQYSSYL |
| | | 835 | 836 | 837 | 838 | 839 | 840 |
| 26 | CD8B369 | TNTYIS | WIAWIYTGTGGTW | ARTNWDWYFD | YSYLAWY | LLVYYAKTLT | QHHYGRPY |
| | | 869 | 870 | 871 | 872 | 873 | 874 |
| 27 | CD8B371 | SDYYMA | WVAHINYDGSITY | AREDYSNYGFA | NVWLSWY | LLIYKASNLH | QQGQSYPL |
| | | 903 | 904 | 905 | 906 | 907 | 908 |
| 28 | CD8B182 | TSYWMN | WIGAVNPTNYYTE | ARSGLYNTNHLAWFA | HNYLAWY | LLVYNAKTLA | QHFWTTPL |
| | | 937 | 938 | 939 | 940 | 941 | 942 |
| 29 | CD8B205 | NSYWMH | WIGNIDPSDSETH | ARVYYSYYSYDATYFD | YSYLAWY | LLVYNAKTLA | QHHYTTPL |
| | | 971 | 972 | 973 | 974 | 975 | 976 |
| 30 | CD8B223 | TSYSVH | WLGVIWAGGSTN | AKHSYYSFDAFD | NTDVAWY | ALIYSASYRY | QQCNSYPL |
| | | 1005 | 1006 | 1007 | 1008 | 1009 | 1010 |
| 31 | CD8B234 | TSGYYWN | WMGYINYDGRNN | SRDQGYSKFYFD | YNRLAWY | LLISGATSLE | QQYWSFPR |
| | | 1039 | 1040 | 1041 | 1042 | 1043 | 1044 |
| 32 | CD8B251 | TTYAVH | WLGVIWSGGSTD | ARHSYYHYNAMD | GTAVAWY | LLIYSASNRY | QQYSSYPF |
| | | 1073 | 1074 | 1075 | 1076 | 1077 | 1078 |
| 33 | CD8B269 | TSGYYWN | WMGYISYDGSNN | VRNHGDAMD | GTDVAWY | ALIYSASYRY | QQYKSYPL |
| | | 1107 | 1108 | 1109 | 1110 | 1111 | 1112 |
| 34 | CD8B290 | SRYSVH | WLGMIWGGGSTD | ARIYFDNYVGFA | GTVVAWY | LLIFWTSTRH | QQYSSYPY |
| | | 1141 | 1142 | 1143 | 1144 | 1145 | 1146 |
| 35 | CD8B310 | TNYAVH | WLGVIWTDGSTD | ARNNGYFPAFFA | VHSNGNTYLEWY | LLMYKVSNRF | FQGSHAPF |
| | | 1175 | 1176 | 1177 | 1178 | 1179 | 1180 |
| 36 | CD8B352 | TSGYYWN | WMGYINYDGRNN | ARDQGYSKFYFD | YNRLAWY | LLISGATSLE | QQYWSFPR |
| | | 1209 | 1210 | 1211 | 1212 | 1213 | 1214 |
| 37 | CD8B319 | TAYYMH | WIGEINPSAGGTT | ARWTNPFD | GTAVAWY | LLIYSASYRY | QQYNNYL |
| | | 1243 | 1244 | 1245 | 1246 | 1247 | 1248 |
| 38 | CD8B194 | TSYWIN | WIGNIYPGSSSTN | ARELGPYYRYSAMV | GTAVAWY | LLIYSASNRY | QQYSSYPF |
| | | 1277 | 1278 | 1279 | 1280 | 1281 | 1282 |
| 39 | CD8B231 | TNYWMH | WIGNIDPSDSETH | ASGLTGTGH | NIYLNWY | CLIYHTSRLH | QQDNTLPY |
| | | 1311 | 1312 | 1313 | 1314 | 1315 | 1316 |
| 40 | CD8B238 | TDYSMD | WIGYIYTYSGGAG | ARDSSDYEFA | KSYLSWF | TLIYRANRLV | LQYDEFR |
| | | 1345 | 1346 | 1347 | 1348 | 1349 | 1350 |
| 41 | CD8B255 | NTSGMGVS | WLAHIFWDDDKR | ARRDGYGDYAYFD | YSDLAWY | LLVYAATILT | QHFWGTPW |
| | | 1379 | 1380 | 1381 | 1382 | 1383 | 1384 |
| 42 | CD8B324 | TSHWIH | WIGNIYPGSSSTN | ARHSPGHRDYAMD | GTAVAWY | LLIASASNRY | QQYSTYPL |
| | | 1413 | 1414 | 1415 | 1416 | 1417 | 1418 |
| 43 | CD8B337 | STSGMGVS | WLAHIFWDDDRR | ARRVGYGDYAYFD | YSDLAWY | LLVYAATNLA | QHFWGTPW |
| | | 1447 | 1448 | 1449 | 1450 | 1451 | 1452 |
| 44 | CD8B344 | TNYWIN | WIGNIYPGSDSSN | AREEADYRYTWFV | GTAVAWY | LLIYSASNRY | QQYSSYPL |
| | | 1481 | 1482 | 1483 | 1484 | 1485 | 1486 |
| 45 | CD8B264 | TSYWIN | WIGNIYPGSSSTN | AREEYSYKSSWFA | GTAVAWY | LLIYSASNRY | QQYSTYPY |
| | | 1515 | 1516 | 1517 | 1518 | 1519 | 1520 |
| 46 | CD8B318 | TSYWIS | WIGNIYPGSSSSN | AREEYSYFPSWFA | GTAVAWF | LLIYSASNRY | QQYSTYPF |
| | | 1549 | 1550 | 1551 | 1552 | 1553 | 1554 |
| 47 | CD8B333 | ASFWIN | WIGNIYPGSSSTN | AREEYSYKSSWFA | GTAVAWY | LLIYSASNRY | QQYSTYPY |
| | | 1583 | 1584 | 1585 | 1586 | 1587 | 1588 |
| 48 | CD8B366 | KDDYIH | WIGRIDPANGNPR | ARDDEGYYYFD | SKYLAWY | VLIYSGSTLQ | QQHNEYPL |
| | | 1617 | 1618 | 1619 | 1620 | 1621 | 1622 |
| 49 | CD8B368 | TSYWIN | WIGNIYPFSSSTN | AREEFSHYPSWFA | GIAVAWF | LLIYSASNRY | QQYSTDPY |
| | | 1651 | 1652 | 1653 | 1654 | 1655 | 1656 |
| 50 | CD8B370 | TSYWIN | WIGNIYPGSSSTN | TRELGAYYHYSAMD | GTAVAWY | LLIYSASNRY | QQYSIYPF |
| | | 1685 | 1686 | 1687 | 1688 | 1689 | 1690 |
| 51 | CD8B186 | TSYWMH | WIGNINPSSGYAV | ARRVFYGDSWFA | HNYLAWY | LLVYNAKTLA | QHFWSTTW |
| | | 1719 | 1720 | 1721 | 1722 | 1723 | 1724 |
| 52 | CD8B190 | TSYYMH | WIGYIDPFNGNTN | ASPNSNYVGTWFA | NVWLSWY | LLIYKASNLH | QQGQSFPF |
| | | 1753 | 1754 | 1755 | 1756 | 1757 | 1758 |
| 53 | CD8B192 | TDYYMN | WIGVINPYNGGTT | ARNYGAMD | HNYLAWY | LLVSNAKTLA | QHFWITPP |
| | | 1787 | 1788 | 1789 | 1790 | 1791 | 1792 |
| 54 | CD8B193 | TDYYMN | WIGDINPNGGGTS | ARTSGTDWYFD | GTAVAWY | LLIYSASNRY | QQYSSYPF |
| | | 1821 | 1822 | 1823 | 1824 | 1825 | 1826 |
| 55 | CD8B214 | TTAGIQ | WIGWINTHAGESK | ARSGDYDGSHPFA | RPYLNWY | LLIYYTSRLH | QQDNTLPY |
| | | 1855 | 1856 | 1857 | 1858 | 1859 | 1860 |
| 56 | CD8B230 | TDYYMN | WIGDINPNGGGTS | ARTSGTDWYFD | GTAVAWY | LLIYSTSNRY | QQYSIYPF |
| | | 1889 | 1890 | 1891 | 1892 | 1893 | 1894 |
| 57 | CD8B245 | TDYYMS | WLALSRNKGNGYTTE | ARTVTGTLFYYALD | YSYLAWY | FLVYNAKTLA | QHHYGTPL |
| | | 1923 | 1924 | 1925 | 1926 | 1927 | 1928 |
| 58 | CD8B248 | TTYTMH | WIGYINPSSGYTK | ARLWA | VHSSGNTYLHWY | LLIYKGSNRF | SQSTHVPF |
| | | 1957 | 1958 | 1959 | 1960 | 1961 | 1962 |
| 59 | CD8B250 | SNYVVH | WLGVIWTDGSTD | ARNNGYFPAFFA | DTDITWY | ALIYSASYRY | QQYNSYPL |
| | | 1991 | 1992 | 1993 | 1994 | 1995 | 1996 |
| 60 | CD8B254 | SSYWIT | WVGDIYPGSGSTN | ARESITTRITPFD | VHSSGNTYLHWY | LLIYKGSNRF | SQSTHVPF |
| | | 2025 | 2026 | 2027 | 2028 | 2029 | 2030 |
| 61 | CD8B261 | NSYWIN | WIGNIYPGSSSTN | ARELGGYYRYNAMD | NRYLSWF | TLIYRANTLV | LQYDEFPY |
| | | 2059 | 2060 | 2061 | 2062 | 2063 | 2064 |
| 62 | CD8B311 | TSYWMH | WIGMIHPNSGSTN | ARCGYDGAWFA | SNCLNWY | LLIHYTSSLH | QQYSKVPY |
| | | 2093 | 2094 | 2095 | 2096 | 2097 | 2098 |
| 63 | CD8B340 | TNYWMQ | WIGEIDPSDTFTN | ARGDWDRDWYFD | LYSDGKTYLNWL | LLIYLVSKLD | LQATHFPH |
| | | 2127 | 2128 | 2129 | 2130 | 2131 | 2132 |
| 64 | CD8B362 | KDTYMH | WIGRIDPANGHTK | AIRFA | SGYLSWL | RLIYAASTLD | LQYSSYPY |
| | | 2161 | 2162 | 2163 | 2164 | 2165 | 2166 |

TABLE 21
IMGT CDR Amino Acid Sequences

| | Protein | | HC IMGT | | | LC IMGT | |
|---|---|---|---|---|---|---|---|
| # | Name | HC IMGT CDR1 | CDR2 | HC IMGT CDR3 | LC IMGT CDR1 | CDR2 | LC IMGT CDR3 |
| 1 | CD8B191 | GYTFTDYY | VIPSNGGT | AREDYNNQGFFLDAMDY | QSISDF | YAS | QNGHSFPYT |
| | | 25 | 26 | 27 | 28 | 29 | 30 |
| 2 | CD8B226 | GYTFTDYY | IIPSNGAT | AREDYSNQGFFLDAMDY | QSISHY | YAS | QNGHSFPYT |
| | | 59 | 60 | 61 | 62 | 63 | 64 |
| 3 | CD8B259 | GYTFTDYY | VIPSNGGT | AREDYGNQGFFLDAMDY | QSISHF | YAS | QSGHSFPYT |
| | | 93 | 94 | 95 | 96 | 97 | 98 |
| 4 | CD8B298 | GYTFTDYY | VIPNNGGT | AREDFSNQGFFLDAMDY | QTISDY | YAS | QNGHSFPYT |
| | | 127 | 128 | 129 | 130 | 131 | 132 |
| 5 | CD8B342 | GYTFTDYY | VIPNNGNV | TREDYSNQGFFLDAMDY | QTISNY | YAS | QNGHSFPYT |
| | | 161 | 162 | 163 | 164 | 165 | 166 |
| 6 | CD8B364 | GYTFTSYW | INPSNGDS | TRSMYYDGRAGAY | TDIDDD | EGN | LQSDNMPLT |
| | | 195 | 196 | 197 | 198 | 199 | 200 |
| 7 | CD8B200 | GYTFTNYW | IDPSDSET | ASGLTGTGYY | QDISPY | YTS | QQDNTLPYT |
| | | 229 | 230 | 231 | 232 | 233 | 234 |
| 8 | CD8B247 | GYTFTDYY | VIPNNGGT | AREDYSNQGFFLDAMDY | QTISHF | YAS | QSGHSFPYT |
| | | 263 | 264 | 265 | 266 | 267 | 268 |
| 9 | CD8B265 | GYSFTDYY | VIPRNGAT | AREDFSNQGFFLDAMDY | QSISHY | YAS | QNGHSFPYT |
| | | 297 | 298 | 299 | 300 | 301 | 302 |
| 10 | CD8B270 | GYTFTNYW | IDPSDSET | ASGLTGTGYY | QDIRPY | FTS | QQDNTLPYT |
| | | 331 | 332 | 333 | 334 | 335 | 336 |
| 11 | CD8B213 | GYIFTDYY | IYPNNGIT | ARSIYYDHGGGFPY | QNVDKY | SAS | QQYNTYPS |
| | | 365 | 366 | 367 | 368 | 369 | 370 |
| 12 | CD8B240 | GYTFTDYY | VIPSNGGT | AREDYNNQGFFLDAMDY | QSISDF | YAS | QNGHSFPYT |
| | | 399 | 400 | 401 | 402 | 403 | 404 |
| 13 | CD8B361 | GYTFTDYY | IYPNNGDT | ARSIYYDHGGGFPY | QNVGTY | SAS | QQYNSYPT |
| | | 433 | 434 | 435 | 436 | 437 | 438 |
| 14 | CD8B246 | GFSLSTSGMN | IWWDDDK | ARRGNYGNYEFAY | QDIRNY | HTS | QQGNTLPWT |
| | | 467 | 468 | 469 | 470 | 471 | 472 |
| 15 | CD8B268 | GYTFTVYT | FYPGSGNI | ARHEDNHYYDGNSWFAY | GNIHNY | NAK | QHFWNTPYT |
| | | 501 | 502 | 503 | 504 | 505 | 506 |
| 16 | CD8B271 | GFSLSIYS | IWGGGDT | ARNPHYYGGTYEYFDV | QGISNY | DTS | QQYSNLPYT |
| | | 535 | 536 | 537 | 538 | 539 | 540 |
| 17 | CD8B273 | GYTFTEYT | FYPGTGSI | ARHEDNHYYDGNSWFAY | GNIHNY | NAK | QHFWSTPYT |
| | | 569 | 570 | 571 | 572 | 573 | 574 |
| 18 | CD8B288 | GYTFTEYT | FYPGNGNM | ARYEDNHYYDGASWFAY | GNIHNY | NAK | QHFWSTPFT |
| | | 603 | 604 | 605 | 606 | 607 | 608 |
| 19 | CD8B292 | GFNFKDDY | IDPENGAT | SLHDYGYAMDY | SSVSSSY | STS | HQYHRSPLT |
| | | 637 | 638 | 639 | 640 | 641 | 642 |
| 20 | CD8B303 | GFSLSIYS | IWGGGST | ARNPHHYGGSTGAMDY | QDIKKY | YTS | LQYDNLFT |
| | | 671 | 672 | 673 | 674 | 675 | 676 |
| 21 | CD8B304 | GFSLSTSGMN | IWWDDDK | ARRGNYGNYEFAY | QDIRNY | HTS | QQGNTLPWT |
| | | 705 | 706 | 707 | 708 | 709 | 710 |
| 22 | CD8B312 | GYTFTSFW | VDPSDSQT | ARSTYYRYDGPFTY | QSINNN | YTS | QQSNSWPLT |
| | | 739 | 740 | 741 | 742 | 743 | 744 |
| 23 | CD8B347 | GYTFTSYW | VNPSNSYT | ARSGLYNTNHLAWFAY | GNIHNY | NAE | QHFWNNPLT |
| | | 773 | 774 | 775 | 776 | 777 | 778 |
| 24 | CD8B350 | GYTFAAYW | INPSNGYT | SRSGLYYTNHLAWCPY | GNIHNY | NAE | QHFWNSPLT |
| | | 807 | 808 | 809 | 810 | 811 | 812 |
| 25 | CD8B356 | GYSITSGYY | ISYDGSN | VRNHGDAMDY | QNVGTA | SAS | QQYSSYLT |
| | | 841 | 842 | 843 | 844 | 845 | 846 |
| 26 | CD8B369 | GFTFTNTY | IYTGTGGT | ARTNWDWYFDV | ENIYSY | YAK | QHHYGRPYT |
| | | 875 | 876 | 877 | 878 | 879 | 880 |
| 27 | CD8B371 | GFTFSDYY | INYDGSIT | AREDYSNYGFAY | QNINVW | KAS | QQGQSYPLT |
| | | 909 | 910 | 911 | 912 | 913 | 914 |
| 28 | CD8B182 | GYTFTSYW | VNPTNYYT | ARSGLYNTNHLAWFAY | ENIHNY | NAK | QHFWTTPLT |
| | | 943 | 944 | 945 | 946 | 947 | 948 |
| 29 | CD8B205 | GYSFNSYW | IDPSDSET | ARVYYSYYSYDATYFDY | ENIYSY | NAK | QHHYTTPLT |
| | | 977 | 978 | 979 | 980 | 981 | 982 |
| 30 | CD8B223 | GFSLTSYS | IWAGGST | AKHSYYS FDAFDY | QNVNTD | SAS | QQCNSYPLT |
| | | 1011 | 1012 | 1013 | 1014 | 1015 | 1016 |
| 31 | CD8B234 | GYSITSGYY | INYDGRN | SRDQGYSKFYFDY | EDIYNR | GAT | QQYWSFPRT |
| | | 1045 | 1046 | 1047 | 1048 | 1049 | 1050 |
| 32 | CD8B251 | GFSLTTYA | IWSGGST | ARHSYYHYNAMDN | QNVGTA | SAS | QQYSSYPFT |
| | | 1079 | 1080 | 1081 | 1082 | 1083 | 1084 |
| 33 | CD8B269 | GYSITSGYY | ISYDGSN | VRNHGDAMDH | QNVGTD | SAS | QQYKSYPLT |
| | | 1113 | 1114 | 1115 | 1116 | 1117 | 1118 |
| 34 | CD8B290 | GFSLSRYS | IWGGGST | ARIYFDNYVGFAY | QDVGTV | WTS | QQYSSYPYT |
| | | 1147 | 1148 | 1149 | 1150 | 1151 | 1152 |
| 35 | CD8B310 | GFSLTNYA | IWTDGST | ARNNGYFPAFFAY | QTIVHSNGNTY | KVS | FQGSHAPFT |
| | | 1181 | 1182 | 1183 | 1184 | 1185 | 1186 |
| 36 | CD8B352 | GYSITSGYY | INYDGRN | ARDQGYSKFYFDY | EDIYNR | GAT | QQYWSFPRT |
| | | 1215 | 1216 | 1217 | 1218 | 1219 | 1220 |
| 37 | CD8B319 | GYSFTAYY | INPSAGGT | ARWTNPFDY | QNVGTA | SAS | QQYNNYLT |
| | | 1249 | 1250 | 1251 | 1252 | 1253 | 1254 |
| 38 | CD8B194 | GYTFTSYW | IYPGSSST | ARELGPYYRYSAMVY | QNVGTA | SAS | QQYSSYPFT |
| | | 1283 | 1284 | 1285 | 1286 | 1287 | 1288 |
| 39 | CD8B231 | GYTFTNYW | IDPSDSET | ASGLTGTGHY | QDINIY | HTS | QQDNTLPYT |
| | | 1317 | 1318 | 1319 | 1320 | 1321 | 1322 |
| 40 | CD8B238 | GYTFTDYS | IYTYSGGA | ARDSSDYEFAY | QDIKSY | RAN | LQYDEFRT |
| | | 1351 | 1352 | 1353 | 1354 | 1355 | 1356 |
| 41 | CD8B255 | GFSLNTSGMG | IFWDDDK | ARRDGYGDYAYFDV | ENIYSD | AAT | QHFWGTPWT |
| | | 1385 | 1386 | 1387 | 1388 | 1389 | 1390 |
| 42 | CD8B324 | GYTSTSHW | IYPGSSST | ARHSPGHRDYAMDY | QNVGTA | SAS | QQYSTYPLT |
| | | 1419 | 1420 | 1421 | 1422 | 1423 | 1424 |
| 43 | CD8B337 | GFSLSTSGMG | IFWDDDR | ARRVGYGDYAYFDV | ENIYSD | AAT | QHFWGTPWT |
| | | 1453 | 1454 | 1455 | 1456 | 1457 | 1458 |
| 44 | CD8B344 | GYSFTNYW | IYPGSDSS | AREEADYRYTWFVY | QNVGTA | SAS | QQYSSYPLT |
| | | 1487 | 1488 | 1489 | 1490 | 1491 | 1492 |
| 45 | CD8B264 | GYSFTSYW | IYPGSSST | AREEYSYKSSWFAY | QNVGTA | SAS | QQYSTYPYT |
| | | 1521 | 1522 | 1523 | 1524 | 1525 | 1526 |
| 46 | CD8B318 | GYTFTSYW | IYPGSSSS | AREEYSYFPSWFAY | QNVGTA | SAS | QQYSTYPFT |
| | | 1555 | 1556 | 1557 | 1558 | 1559 | 1560 |
| 47 | CD8B333 | GYSFASFW | IYPGSSST | AREEYSYKSSWFAY | QNVGTA | SAS | QQYSTYPYT |
| | | 1589 | 1590 | 1591 | 1592 | 1593 | 1594 |
| 48 | CD8B366 | GFNIKDDY | IDPANGNP | ARDDEGYYYFDV | KSISKY | SGS | QQHNEYPLT |
| | | 1623 | 1624 | 1625 | 1626 | 1627 | 1628 |
| 49 | CD8B368 | GYTFTSYW | IYPFSSST | AREEFSHYPSWFAY | QNVGIA | SAS | QQYSTDPYT |
| | | 1657 | 1658 | 1659 | 1660 | 1661 | 1662 |
| 50 | CD8B370 | GYTFTSYW | IYPGSSST | TRELGAYYHYSAMDY | QNVGTA | SAS | QQYSIYPFT |
| | | 1691 | 1692 | 1693 | 1694 | 1695 | 1696 |
| 51 | CD8B186 | GYIFTSYW | INPSSGYA | ARRVFYGDSWFAY | GNIHNY | NAK | QHFWSTTWT |
| | | 1725 | 1726 | 1727 | 1728 | 1729 | 1730 |
| 52 | CD8B190 | GYSFTSYY | IDPFNGNT | ASPNSNYVGTWFAY | QNINVW | KAS | QQGQSFPFT |
| | | 1759 | 1760 | 1761 | 1762 | 1763 | 1764 |
| 53 | CD8B192 | GYTFTDYY | INPYNGGT | ARNYGAMDS | GNIHNY | NAK | QHFWITPPT |
| | | 1793 | 1794 | 1795 | 1796 | 1797 | 1798 |
| 54 | CD8B193 | GYKFTDYY | INPNGGGT | ARTSGTDWYFDV | QNVGTA | SAS | QQYSSYPFT |
| | | 1827 | 1828 | 1829 | 1830 | 1831 | 1832 |
| 55 | CD8B214 | GYTFTTAG | INTHAGES | ARSGDYDGSHPFAY | QDIRPY | YTS | QQDNTLPYT |
| | | 1861 | 1862 | 1863 | 1864 | 1865 | 1866 |
| 56 | CD8B230 | GYTFTDYY | INPNGGGT | ARTSGTDWYFDV | QNVGTA | STS | QQYSIYPFT |
| | | 1895 | 1896 | 1897 | 1898 | 1899 | 1900 |
| 57 | CD8B245 | GFTFTDYY | SRNKGNGYTT | ARTVTGTLFYYALDY | ENIYSY | NAK | QHHYGTPLT |
| | | 1929 | 1930 | 1931 | 1932 | 1933 | 1934 |
| 58 | CD8B248 | GYTFTTYT | INPSSGYT | ARLWAY | QSLVHSSGNTY | KGS | SQSTHVPFT |
| | | 1963 | 1964 | 1965 | 1966 | 1967 | 1968 |
| 59 | CD8B250 | GFSLSNYV | IWTDGST | ARNNGYFPAFFAY | QNVDTD | SAS | QQYNSYPLT |
| | | 1997 | 1998 | 1999 | 2000 | 2001 | 2002 |
| 60 | CD8B254 | GYTFSSYW | IYPGSGST | ARESITTRITPFDH | QSLVHSSGNTY | KGS | SQSTHVPFT |
| | | 2031 | 2032 | 2033 | 2034 | 2035 | 2036 |
| 61 | CD8B261 | GYTFNSYW | IYPGSSST | ARELGGYYRYNAMDY | QDINRY | RAN | LQYDEFPYT |
| | | 2065 | 2066 | 2067 | 2068 | 2069 | 2070 |
| 62 | CD8B311 | GYTFTSYW | IHPNSGST | ARCGYDGAWFAY | QGISNC | YTS | QQYSKVPYT |
| | | 2099 | 2100 | 2101 | 2102 | 2103 | 2104 |
| 63 | CD8B340 | GYTFTNYW | IDPSDTFT | ARGDWDRDWYFDV | QSLLYSDGKTY | LVS | LQATHFPHT |
| | | 2133 | 2134 | 2135 | 2136 | 2137 | 2138 |
| 64 | CD8B362 | GFNIKDTY | IDPANGHT | AIRFAY | HEISGY | AAS | LQYSSYPYT |
| | | 2167 | 2168 | 2169 | 2170 | 2171 | 2172 |

4.2: Evaluation of Binding to Human CD8+ T Cells and Biophysical Characterization of CD8 Antibodies

Cell binding: Twenty nM antibody was incubated with human pan T cell in assay media (RPMI 1640+10% HI FBS+ Pen/strep) for 1 hour at 37° C. Secondary antibodies were A647-conjugated goat anti human IgG Fc antibody at 2 μg/mL, and A488-conjugated mouse anti-human CD4 at 1 μg/mL in staining buffer. Live cells were also gated based on OKT8 control mAb binding. Percent CD8 positive population was calculated by percentage of CD8-positive cell count/live cell count. Results are shown in Table 22 and are reported as Geomean ratios from CD4-negative population (% CD8-positive population).

Cross-interaction chromatography (CIC): CIC was conducted as previously described (Jacobs et al. (2010) Pharm. Res. 27(1):65-71). Results are shown in Table 22.

Thermal unfolding and aggregation (Tm/Tagg): Thermal unfolding and aggregation was measured 20° C.-95° C. in 1 C/min ramp using Nanodsf Nanotemper's PROMETHIUSNT.48 instrument. Samples of 20 μL (0.2 mg/mL) in PBS buffer were transferred to 384-well plate in duplicate. Data was analyzed using PR.THERMCONTROL software. Results are shown in Table 22.

TABLE 22
Antibody Stability and Binding to Human Pan T Cells

| | | Cell Binding | | | |
|---|---|---|---|---|---|
| | | to Human PanT | CIC | | |
| | | Signal/Background | Peak | Protein Stability | |
| | Protein | (of CD4 negative | Retention | Tm1 | Tagg |
| # | Name | population) | Time | (° C.) | (° C.) |
| 1 | CD8B191 | 2440 | 4.32 | 70.3 | 76.6 |
| 2 | CD8B226 | 1752 | 4.34 | 70.2 | 78.0 |
| 3 | CD8B259 | 1934 | 4.41 | 70.5 | 76.8 |
| 4 | CD8B298 | 306 | 4.29 | 70.6 | 76.2 |
| 5 | CD8B342 | 1324 | 4.27 | 67.5 | 68.7 |
| 6 | CD8B364 | 1562 | 4.24 | 65.3 | 70.7 |
| 7 | CD8B200 | 1990 | 4.23 | 69.3 | 82.3 |
| 8 | CD8B247 | 1646 | 4.31 | 70.1 | 77.4 |
| 9 | CD8B265 | 2076 | 4.39 | 70.3 | 79.0 |
| 10 | CD8B270 | 2497 | 4.32 | 70.1 | 79.7 |
| 11 | CD8B213 | 827 | 4.51 | 67.8 | 69.9 |
| 12 | CD8B240 | 1312 | 4.30 | 70.0 | 81.5 |
| 13 | CD8B361 | 1051 | 4.65 | 71.1 | 74.4 |
| 14 | CD8B246 | 1112 | 4.47 | 60.9 | 63.1 |
| 15 | CD8B268 | 1173 | 4.44 | 69.6 | 72.4 |
| 16 | CD8B271 | 911 | 4.34 | 69.1 | 80.4 |
| 17 | CD8B273 | 938 | 4.27 | 73.0 | 76.9 |
| 18 | CD8B288 | 934 | 4.32 | 71.0 | 73.5 |
| 19 | CD8B292 | 910 | 4.23 | 68.1 | 69.2 |
| 20 | CD8B303 | 1182 | 4.37 | 70.2 | 79.9 |
| 21 | CD8B304 | 923 | 4.43 | 64.4 | 66.4 |
| 22 | CD8B312 | 1087 | 4.29 | 71.3 | 78.0 |
| 23 | CD8B347 | 1201 | 4.30 | 71.1 | 73.1 |
| 24 | CD8B350 | 537 | 4.61 | | 81.3 |
| 25 | CD8B356 | 777 | 4.46 | 73.9 | 76.7 |
| 26 | CD8B369 | 685 | 5.83 | 67.4 | 76.2 |
| 27 | CD8B371 | 64 | 4.29 | 69.1 | 75.0 |
| 28 | CD8B182 | 1490 | 4.58 | 70.7 | 77.8 |
| 29 | CD8B205 | 655 | 4.77 | 68.9 | 72.2 |
| 30 | CD8B223 | 489 | 4.46 | 68.3 | 74.3 |
| 31 | CD8B234 | 856 | 5.16 | 67.7 | 69.0 |
| 32 | CD8B251 | 37 | 5.30 | 69.4 | 73.0 |
| 33 | CD8B269 | 26 | 4.28 | 69.8 | 81.4 |
| 34 | CD8B290 | 1155 | 4.48 | 60.5 | 72.0 |
| 35 | CD8B310 | 29 | 4.32 | 70.7 | 78.7 |
| 36 | CD8B352 | 827 | 5.56 | 72.1 | 72.6 |
| 37 | CD8B319 | 16 | 4.54 | 64.8 | 75.6 |
| 38 | CD8B194 | 1972 | 4.81 | 69.8 | 87.2 |
| 39 | CD8B231 | 1785 | 4.19 | 61.7 | 77.5 |
| 40 | CD8B238 | 1 | 4.38 | 69.9 | 78.3 |
| 41 | CD8B255 | 1317 | 4.25 | 69.5 | 78.4 |
| 42 | CD8B324 | 1611 | 4.44 | 66.9 | 68.9 |
| 43 | CD8B337 | 1983 | 4.42 | 68.8 | 73.2 |
| 44 | CD8B344 | 1758 | 4.26 | 72.4 | 75.4 |
| 45 | CD8B264 | 122 | 4.34 | 70.0 | 87.2 |
| 46 | CD8B318 | 1613 | 4.78 | | 78.0 |
| 47 | CD8B333 | 1843 | 4.24 | 70.4 | 85.0 |
| 48 | CD8B366 | 318 | 4.26 | 71.8 | 74.9 |
| 49 | CD8B368 | 2007 | 4.46 | 70.5 | 74.7 |
| 50 | CD8B370 | 1932 | 4.69 | 70.1 | 86.9 |
| 51 | CD8B186 | 36 | 4.94 | 65.1 | 66.4 |
| 52 | CD8B190 | 44 | 4.34 | 67.9 | 77.0 |
| 53 | CD8B192 | 22 | 4.84 | 70.2 | 79.9 |
| 54 | CD8B193 | 641 | 5.48 | 70.3 | 79.6 |
| 55 | CD8B214 | 232 | 4.16 | 68.1 | 73.9 |
| 56 | CD8B230 | 63 | 4.88 | 69.6 | 82.5 |
| 57 | CD8B245 | 44 | 4.36 | 66.7 | 68.3 |
| 58 | CD8B248 | 20 | 4.57 | 68.4 | 73.8 |
| 59 | CD8B250 | 61 | 4.42 | 69.9 | 79.3 |
| 60 | CD8B254 | 23 | 4.22 | 65.8 | 69.8 |
| 61 | CD8B261 | 34 | 4.52 | 70.5 | 79.0 |
| 62 | CD8B311 | 1 | 4.28 | 69.8 | 78.0 |
| 63 | CD8B340 | 8 | 4.21 | 64.8 | 78.0 |
| 64 | CD8B362 | 4 | 4.37 | 69.6 | 76.0 |

Protein binding kinetics by surface plasmon resonance (SPR). All 64 mAbs were captured at 1 μg/ml, with a final capture level ranging from 100 to 400 Rus. Binding to human CD8αβ heterodimer (R&D cat #9358-CD) and hCD8αα homodimer (Table 23) at 11.1 nM, 33.3 nM and 100 nM was measured using a single cycle kinetics method with an association and dissociation of 3 and 10 minutes, respectively, using a flow rate of 50 μL/mL. Biacore 8k was utilized for these assays, and data was analyzed by modeling to a 1:1 binding equation. Results are shown in Table 24.

TABLE 23
CD8αα screening reagents

| | | | SEQ |
|---|---|---|---|
| | Protein | | ID |
| Name | ID | Sequence | NO |
| Human | hCDaa | MAWVWTLLFLMAAAQSIQASQFRVSPLDR | 2179 |
| CD8αα | | TWNLGETVELKCQVLLSNPTSGCSWLFQP | |
| fused to | | RGAAASPTFLLYLSQNKPKAAEGLDTQRF | |
| human Fc | | SGKRLGDTFVLTLSDFRRENEGYYFCSAL | |
| | | SNSIMYFSHFVPVFLPAKPTTTPAPRPPT | |
| | | PAPTIASQPLSLRPEACRPAAGGAVHTRG | |
| | | LDFACDEPKSCDKTHTCPPCPAPELLGGP | |
| | | SVFLFPPKPKDTLMISRTPEVICVVVDVS | |
| | | HEDPEVKFNWYVDGVEVHNAKTKPREEQY | |
| | | NSTYRVVSVLTVLHQDWLNGKEYKCKVSN | |
| | | KALPAPIEKTISKAKGQPREPQVYTLPPS | |
| | | RDELTKNQVSLTCLVKGFYPSDIAVEWES | |
| | | NGQPENNYKTTPPVLDSDGSFFLYSKLTV | |
| | | DKSRWQQGNVFSCSVMHEALHNHYTQKSL | |
| | | SLSPGK | |

TABLE 24
Antibody Binding by SPR

| | | | | | | Protein binding by SPR | | | | |
|---|---|---|---|---|---|---|---|---|---|---|
| | | Protein binding by SPR | | | | to human CD8 αβ heterodimer | | | | Based on |
| | | to human CD8αα homodimer | | | | hCD8αβ | hCD8α | hCD8αβ | | SPR Data |
| | Protein | ka | kd | KD | | ka | kd | KD | hCD8αβ | Predicted |
| # | Name | (1/Ms) | (1/s) | (M) | Comment | (1/Ms) | (1/s) | (M) | Comment | Epitope |
| 1 | CD8B191 | 1.23E+05 | 1.19E−04 | 9.68E−10 | | 1.23E+05 | 1.19E−04 | 9.68E−10 | | CD8 α |
| 2 | CD8B226 | 1.55E+05 | 3.42E−04 | 2.21E−09 | | 1.55E+05 | 3.42E−04 | 2.21E−09 | | CD8 α |
| 3 | CD8B259 | 2.09E+05 | 2.52E−04 | 1.20E−09 | | 2.09E+05 | 2.52E−04 | 1.20E−09 | | CD8 α |
| 4 | CD8B298 | 1.32E+05 | 2.11E−04 | 1.60E−09 | | 1.32E+05 | 2.11E−04 | 1.60E−09 | | CD8 α |
| 5 | CD8B342 | 1.48E+05 | 3.84E−04 | 2.59E−09 | | 1.48E+05 | 3.84E−04 | 2.59E−09 | | CD8 α |
| 6 | CD8B364 | 1.43E+06 | 3.12E−02 | 2.19E−08 | | 1.43E+06 | 3.12E−02 | 2.19E−08 | | CD8 α |
| 7 | CD8B200 | 3.32E+06 | 1.26E−04 | 3.80E−11 | | 3.32E+06 | 1.26E−04 | 3.80E−11 | | CD8 α |
| 8 | CD8B247 | 2.73E+05 | 2.81E−04 | 1.03E−09 | | 2.73E+05 | 2.81E−04 | 1.03E−09 | | CD8 α |
| 9 | CD8B265 | 1.68E+05 | 1.33E−04 | 7.91E−10 | | 1.68E+05 | 1.33E−04 | 7.91E−10 | | CD8 α |
| 10 | CD8B270 | 2.41E+06 | 9.47E−05 | 3.93E−11 | | 2.41E+06 | 9.47E−05 | 3.93E−11 | | CD8 α |
| 11 | CD8B213 | — | — | — | Poor Fit, ~5 | — | — | — | Poor Fit, ~5 | CD8 α |
| | | | | | nM | | | | nM | |
| 12 | CD8B240 | — | — | — | Poor Fit, ~1 | — | — | — | Poor Fit, ~1 | CD8 α |
| | | | | | nM | | | | nM | |
| 13 | CD8B361 | — | — | — | Poor Fit, ~1 | — | — | — | Poor Fit, ~1 | CD8 α |
| | | | | | nM | | | | nM | |
| 14 | CD8B246 | — | — | — | Low/No | — | — | — | Low/No | CD8 β |
| | | | | | Binding | | | | Binding | |
| 15 | CD8B268 | — | — | — | Low/No | — | — | — | Low/No | CD8 β |
| | | | | | Binding | | | | Binding | |
| 16 | CD8B271 | — | — | — | Low/No | — | — | — | Low/No | CD8 β |
| | | | | | Binding | | | | Binding | |
| 17 | CD8B273 | — | — | — | Low/No | — | — | — | Low/No | CD8 β |
| | | | | | Binding | | | | Binding | |
| 18 | CD8B288 | — | — | — | Low/No | — | — | — | Low/No | CD8 β |
| | | | | | Binding | | | | Binding | |
| 19 | CD8B292 | — | — | — | Low/No | — | — | — | Low/No | CD8 β |
| | | | | | Binding | | | | Binding | |
| 20 | CD8B303 | — | — | — | Low/No | — | — | — | Low/No | CD8 β |
| | | | | | Binding | | | | Binding | |
| 21 | CD8B304 | — | — | — | Low/No | — | — | — | Low/No | CD8 β |
| | | | | | Binding | | | | Binding | |
| 22 | CD8B312 | — | — | — | Low/No | — | — | — | Low/No | CD8 β |
| | | | | | Binding | | | | Binding | |
| 23 | CD8B347 | — | — | — | Low/No | — | — | — | Low/No | CD8 β |
| | | | | | Binding | | | | Binding | |
| 24 | CD8B350 | — | — | — | Low/No | — | — | — | Low/No | CD8 β |
| | | | | | Binding | | | | Binding | |
| 25 | CD8B356 | — | — | — | Low/No | — | — | — | Low/No | CD8 β |
| | | | | | Binding | | | | Binding | |
| 26 | CD8B369 | — | — | — | Low/No | — | — | — | Low/No | CD8 β |
| | | | | | Binding | | | | Binding | |
| 27 | CD8B371 | — | — | — | Low/No | — | — | — | Low/No | CD8 β |
| | | | | | Binding | | | | Binding | |
| 28 | CD8B182 | — | — | — | Low/No | — | — | — | Low/No | CD8 β |
| | | | | | Binding | | | | Binding | |
| 29 | CD8B205 | — | — | — | Low/No | — | — | — | Low/No | CD8 β |
| | | | | | Binding | | | | Binding | |
| 30 | CD8B223 | — | — | — | Low/No | — | — | — | Low/No | CD8 β |
| | | | | | Binding | | | | Binding | |
| 31 | CD8B234 | — | — | — | Low/No | — | — | — | Low/No | CD8 β |
| | | | | | Binding | | | | Binding | |
| 32 | CD8B251 | — | — | — | Low/No | — | — | — | Low/No | CD8 β |
| | | | | | Binding | | | | Binding | |
| 33 | CD8B269 | — | — | — | Low/No | — | — | — | Low/No | CD8 β |
| | | | | | Binding | | | | Binding | |
| 34 | CD8B290 | — | — | — | Low/No | — | — | — | Low/No | CD8 β |
| | | | | | Binding | | | | Binding | |
| 35 | CD8B310 | — | — | — | Low/No | — | — | — | Low/No | CD8 β |
| | | | | | Binding | | | | Binding | |
| 36 | CD8B352 | — | — | — | Low/No | — | — | — | Low/No | CD8 β |
| | | | | | Binding | | | | Binding | |
| 37 | CD8B319 | — | — | — | Low/No | — | — | — | Low/No | CD8 β |
| | | | | | Binding | | | | Binding | |
| 38 | CD8B194 | — | — | — | Poor Fit, ~1 | — | — | — | Poor Fit, ~1 | CD8 α/β |
| | | | | | nM | | | | nM | interface |
| 39 | CD8B231 | — | — | — | Poor Fit, ~0.5 | — | — | — | Poor Fit, ~0.5 | CD8 α/β |
| | | | | | nM | | | | nM | interface |
| 40 | CD8B238 | — | — | — | Poor Fit, ~200 | — | — | — | Poor Fit, ~200 | CD8 α/β |
| | | | | | pM | | | | pM | interface |
| 41 | CD8B255 | — | — | — | Poor Fit, ~1 | — | — | — | Poor Fit, ~1 | CD8 α/β |
| | | | | | nM | | | | nM | interface |
| 42 | CD8B324 | — | — | — | Poor Fit, ~1 | — | — | — | Poor Fit, ~1 | CD8 α/β |
| | | | | | nM | | | | nM | interface |
| 43 | CD8B337 | — | — | — | Poor Fit, ~1 | — | — | — | Poor Fit, ~1 | CD8 α/β |
| | | | | | nM | | | | nM | interface |
| 44 | CD8B344 | — | — | — | Poor Fit, ~5 | — | — | — | Poor Fit, ~5 | CD8 α/β |
| | | | | | nM | | | | nM | interface |
| 45 | CD8B264 | — | — | — | Poor Fit, ~0.5 | — | — | — | Poor Fit, ~0.5 | CD8 α/β |
| | | | | | nM | | | | nM | interface |
| 46 | CD8B318 | — | — | — | Poor Fit, ~1 | — | — | — | Poor Fit, ~1 | CD8 α/β |
| | | | | | nM | | | | nM | interface |
| 47 | CD8B333 | — | — | — | Poor Fit, ~1 | — | — | — | Poor Fit, ~1 | CD8 α/β |
| | | | | | nM | | | | nM | interface |
| 48 | CD8B366 | — | — | — | Poor Fit, ~20 | — | — | — | Poor Fit, ~20 | CD8 α/β |
| | | | | | nM | | | | nM | interface |
| 49 | CD8B368 | — | — | — | Poor Fit, ~0.5 | — | — | — | Poor Fit, ~0.5 | CD8 α/β |
| | | | | | nM | | | | nM | interface |
| 50 | CD8B370 | — | — | — | Poor Fit, ~5 | — | — | — | Poor Fit, ~5 | CD8 α/β |
| | | | | | nM | | | | nM | interface |
| 51 | CD8B186 | — | — | — | Low/No | — | — | — | Low/No | — |
| | | | | | Binding | | | | Binding | |
| 52 | CD8B190 | — | — | — | Low/No | — | — | — | Low/No | — |
| | | | | | Binding | | | | Binding | |
| 53 | CD8B192 | — | — | — | Low/No | — | — | — | Low/No | — |
| | | | | | Binding | | | | Binding | |
| 54 | CD8B193 | — | — | — | Low/No | — | — | — | Low/No | — |
| | | | | | Binding | | | | Binding | |
| 55 | CD8B214 | — | — | — | Low/No | — | — | — | Low/No | — |
| | | | | | Binding | | | | Binding | |
| 56 | CD8B230 | — | — | — | Low/No | — | — | — | Low/No | — |
| | | | | | Binding | | | | Binding | |
| 57 | CD8B245 | — | — | — | Low/No | — | — | — | Low/No | — |
| | | | | | Binding | | | | Binding | |
| 58 | CD8B248 | — | — | — | Low/No | — | — | — | Low/No | — |
| | | | | | Binding | | | | Binding | |
| 59 | CD8B250 | — | — | — | Low/No | — | — | — | Low/No | — |
| | | | | | Binding | | | | Binding | |
| 60 | CD8B254 | — | — | — | Low/No | — | — | — | Low/No | — |
| | | | | | Binding | | | | Binding | |
| 61 | CD8B261 | — | — | — | Low/No | — | — | — | Low/No | — |
| | | | | | Binding | | | | Binding | |
| 62 | CD8B311 | — | — | — | Low/No | — | — | — | Low/No | — |
| | | | | | Binding | | | | Binding | |
| 63 | CD8B340 | — | — | — | Low/No | — | — | — | Low/No | — |
| | | | | | Binding | | | | Binding | |
| 64 | CD8B362 | — | — | — | Low/No | — | — | — | Low/No | — |
| | | | | | Binding | | | | Binding | |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An isolated molecule, comprising: a first antigen binding domain and a second antigen binding domain, wherein the first antigen binding domain specifically binds CD8 and the second antigen binding domain specifically binds a T cell receptor (TCR) complex.

2. The isolated molecule of claim 1, further comprising a third antigen binding domain that specifically binds a third antigen, wherein optionally the third antigen comprises an antigen expressed by an undesired cells.

3. (canceled)

4. The isolated molecule of claim 1, wherein the isolated molecule activates or recruits CD8⁺ cytotoxic T lymphocytes (CTLs) upon co-engagement of the TCR complex and CD8, wherein optionally the isolated molecule is unable to activate or recruit CD8⁺ CTLs in the absence of co-engagement of the TCR complex and CD8.

5. (canceled)

6. The isolated molecule of claim 1, wherein the first antigen binding domain specifically binds CD8 and the second antigen binding domain specifically binds the TCR complex with affinities that result in activation or recruitment of CD8⁺ CTLs only upon co-engagement of the TCR complex and CD8.

7. The isolated molecule of claim 1, wherein the first antigen binding domain, the second antigen binding domain or the third antigen binding domain comprises a scFv, a Fab, a Fab′, a F(ab′)₂, a Fd, a Fv, a domain antibody (dAb), a VHH, a heavy chain variable domain (VH), a light chain variable domain (VL), a non-antibody scaffold, or fragments thereof, wherein optionally the first antigen binding domain comprises the Fab, wherein optionally the second antigen binding domain comprises the scFv, and wherein optionally the third antigen binding domain comprises the scFv.

8.-10. (canceled)

11. The isolated molecule of claim 1, comprising:
(I) a) a first polypeptide comprising, from N- to C-terminus, the second antigen binding domain comprising the scFv, a VH that is capable of specifically binding CD8, a CH1 domain, a hinge, a CH2 domain and a CH3 domain;
b) a second polypeptide comprising, from N- to C-terminus, a VL that is capable of specifically binding CD8 and a CL domain; and
c) a third polypeptide comprising, from N- to C-terminus, the third antigen binding domain comprising the scFv and a Fc or a fragment of the Fc; or
(II) a) a first polypeptide comprising, from N- to C-terminus, a VH that is capable of specifically binding CD8, a CH1 domain, a hinge, a CH2 domain and a CH3 domain;
b) a second polypeptide comprising, from N- to C-terminus, a VL that is capable of specifically binding CD8, a CL domain and the second antigen binding domain comprising the scFv; and
c) a third polypeptide comprising, from N- to C-terminus, the third antigen binding domain comprising the scFv and a Fc or a fragment of the Fc; or
(III) a) a first polypeptide comprising, from N- to C-terminus, a VH that is capable of specifically binding CD8, a CH1 domain, a hinge, a CH2 domain, a CH3 domain and the second antigen binding domain comprising the scFv;
b) a second polypeptide comprising, from N- to C-terminus, a VL that is capable of specifically binding CD8 and a CL domain; and
c) a third polypeptide comprising, from N- to C-terminus, the third antigen binding domain comprising the scFv and a Fc or a fragment of the Fc.

12.-13. (canceled)

14. The isolated molecule of claim 11, wherein the first antigen binding domain comprising the Fab, the second antigen binding domain comprising the scFv or the third antigen binding domain comprising the scFv is conjugated to the Fc or the fragment of the Fc, to the VH that is capable of specifically biding CD8, to the CL domain or to the CH3 domain via a linker, wherein optionally the linker comprises a polypeptide of SEQ ID NOs: 2183-2290.

15. (canceled)

16. The isolated molecule of claim 11, wherein the fragment of the Fc comprises a CH2 domain and a CH3 domain, wherein optionally the CH2 domain or the CH3 domain is an IgG1, IgG2, IgG3 or IgG4 isotype.

17. The isolated molecule of claim 16, wherein the CH3 domain comprises one or more substitutions when compared to a wild-type CH3 domain, wherein optionally
a) the first polypeptide comprises a CH3 domain comprising one or more substitutions when compared to a wild-type CH3 domain which promote heterodimerization of the first polypeptide with the third polypeptide,
b) the third polypeptide comprises a CH3 domain comprising one or more substitutions when compared to the wild-type CH3 domain which promote heterodimerization of the third polypeptide with the first polypeptide, or
c) the first polypeptide comprises the CH3 domain comprising one or more substitutions when compared to the wild-type CH3 which promote heterodimerization of the first polypeptide with the third polypeptide and the third polypeptide comprises the CH3 domain comprising one or more substitutions when compared to the wild-type CH3 which promote heterodimerization of the third polypeptide with the first polypeptide,
wherein, optionally, the one or more substitutions comprise T350V, L351Y, F405A, Y407V, T366Y, T366W, F405W, T394W, T394S, Y407T, Y407A, T366S/L368A/Y407V, L351Y/F405A/Y407V, T366I/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V or T350V/T366L/K392L/T394W, wherein residue numbering is according to the EU index.

18. (canceled)

19. The isolated molecule of claim 1, comprising: a first polypeptide, a second polypeptide and a third polypeptide, wherein:
(I) a) the first polypeptide comprises, from N- to C-terminus, a second antigen binding domain comprising a scFv that specifically binds a TCR complex, a VH that is capable of specifically binding CD8, a CH1 domain, a hinge, a CH2 domain and a CH3 domain;
b) the second polypeptide comprises, from N- to C-terminus, a VL that is capable of specifically binding CD8 and a CL domain; and
c) the third polypeptide comprises, from N- to C-terminus, a third antigen binding domain comprising a scFv that specifically binds an antigen expressed by an undesired cell and a Fc or a fragment of the Fc; or
(II) a) the first polypeptide comprises, from N- to C-terminus, a VH that is capable of specifically binding CD8, a CH1 domain, a hinge, a CH2 domain and a CH3 domain;
b) the second polypeptide comprises, from N- to C-terminus, a VL that is capable of specifically binding CD8, a CL domain and a second antigen binding domain comprising a scFv that specifically binds a TCR complex; and
c) the third polypeptide comprises, from N- to C-terminus, a third antigen binding domain comprising a scFv that specifically binds an antigen expressed by an undesired cell and a Fc or a fragment of the Fc; or
(III) a) the first polypeptide comprises, from N- to C-terminus, a VH that is capable of specifically CD8, a CH1 domain, a hinge, a CH2 domain, a CH3 domain and a second antigen binding domain comprising a scFv that specifically binds a TCR complex;
b) the second polypeptide comprises, from N- to C-terminus, a VL that is capable of specifically binding CD8 and a CL domain; and
c) the third polypeptide comprises, from N- to C-terminus, a third antigen binding domain comprising a scFv that specifically binds an antigen expressed by an undesired cell and a Fc or a fragment of the Fc.

20.-21. (canceled)

22. The isolated molecule of claim 19, wherein the first antigen binding domain comprising the Fab, the second antigen binding domain comprising the scFv or the third antigen binding domain comprising the scFv is conjugated to the Fc or the fragment of the Fc, to the VH that is capable of specifically biding CD8, to the CL domain or to the CH3 domain via a linker, wherein optionally the linker comprises a polypeptide of SEQ ID NOs: 2183-2290.

23. (canceled)

24. The isolated molecule of claim 19, wherein
a) the first polypeptide comprises a CH3 domain comprising one or more substitutions when compared to a wild-type CH3 domain which promote heterodimerization of the first polypeptide with the third polypeptide;
b) the third polypeptide comprises a CH3 domain comprising one or more substitutions when compared to the wild-type CH3 domain which promote heterodimerization of the third polypeptide with the first polypeptide; or
c) the first polypeptide comprises the CH3 domain comprising one or more substitutions when compared to the wild-type CH3 which promote heterodimerization of the first polypeptide with the third polypeptide and the third polypeptide comprises the CH3 domain comprising one or more substitutions when compared to the wild-type CH3 which promote heterodimerization of the third polypeptide with the first polypeptide,
wherein, optionally, the one or more substitutions comprise T350V, L351Y, F405A, Y407V, T366Y, T366W, F405W, T394W, T394S, Y407T, Y407A, T366S/L368A/Y407V, L351Y/F405A/Y407V, T366I/K392M/T394W, F405A/Y407V, T366L/K392M/T394W, L351Y/Y407A, T366A/K409F, L351Y/Y407A, T366V/K409F, T366A/K409F, T350V/L351Y/F405A/Y407V or T350V/T366L/K392L/T394W, wherein residue numbering is according to the EU index.

25. (canceled)

26. The isolated molecule of claim 19, wherein the Fc, the CH2 domain or the CH3 domain is an IgG1, IgG2, IgG3 or IgG4 isotype.

27. The isolated molecule of claim 1, wherein the second antigen binding domain specifically binds CD3, TCRα chain, TCRβ chain, TCRγ chain or TCR chain, or any combination thereof.

28. The isolated molecule of claim 27, wherein the TCRβ chain comprises TCRVB17.

29. The isolated molecule of claim 27, wherein CD3 comprises CD3ε, CD3γ, CD3δ or CD3.

30. The isolated molecule of claim 29, wherein the second antigen binding domain that specifically binds CD3 comprises a heavy chain complementarity determining region 1 (HCDR1 of SEQ ID NO: 2291, a HCDR2 of SEQ ID NO: 2292, a HCDR3 of SEQ ID NO: 2293, a LCDR1 of SEQ ID NO: 2294, a LCDR2 of SEQ ID NO: 2295 and a LCDR3 of SEQ ID NO: 2296.

31. The isolated molecule of claim 30, wherein the second antigen binding domain that specifically binds CD3 comprises the VH of SEQ ID NO: 2297 and the VL of SEQ ID NO: 2298.

32. The isolated molecule of claim 1, wherein the first antigen binding domain comprises the HCDR1 of SEQ ID NO: 2307, the HCDR2 of SEQ ID NO: 2308, the HCDR3 of SEQ ID NO: 2309, the LCDR1 of SEQ ID NO: 2310, the LCDR2 of SEQ ID NO: 2311 and the LCDR3 of SEQ ID NO: 2312.

33. The isolated molecule of claim 1, wherein the first antigen binding domain comprises the VH of SEQ ID NO: 2313 and the VL of SEQ ID NO: 2314.

34. The isolated molecule of claim 1, wherein the undesired cell is a pathogenic cell, wherein optionally the undesired cell is a cancer cell, an infected cell, a virus infected cell, a bacterial infected cell, an immune cell, an inflamed cell, a damaged cells, a foreign cell, an apoptotic cell, a dysplastic cell, an immunogenic cell, a metaplastic cell or a mutant cell, or any combination thereof.

35. (canceled)

36. The isolated molecule of claim 1, wherein the isolated molecule is an antibody or a non-antibody molecule, wherein optionally the antibody comprises a first half molecule and a second half molecule, wherein optionally the first half molecule comprises the first antigen binding domain and the second antigen binding domain and the second half molecule comprises the third antigen binding domain.

37. (canceled)

38. The isolated molecule of claim 1, wherein the antigen expressed by the undesired cell comprises mesothelin, alpha-fetoprotein (ALP), BAGE, BCR-ABL, beta-catenin, beta-HCG, BrE3-antigen, BCA225, BCMA, BTAA, CA125, CA195, CA242, CA-50, CAM43, CAMEL, CAP-1, carbonic anhydrase IX, CA19-9, CA72-4, CAM 17.1, CASP-8, CCCL19, CCCL21, CD1, CD 1a, CD2, CD4, CD5, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD47, CD52, CD54, CD55, CD59, CD64, CD66a-e, CD67, CD68, CD70, CD70L, CD74, CD79a, CD79b, CD80, CD83, CD95, CD123, CD126, CD132, CD133, CD138, CD147, CD154, CDC27, CDK4, CDK4m, CDKN2A, CO-029, CTLA4, CXCR4, CXCR7, CXCL12, HIF-1a, colon-specific antigen-p (CSAp), CEACAM5) CEACAM6, c-Met, DAM, E2A-PRL, EGFR, EGFRvIII, EGP-1, EGP-2, ELF2-M, Ep-CAM, FGF, FGF-5, Flt-1, Flt-3, folate receptor, G250 antigen, Ga733VEpCAM, GAGE, gplOO, GRO-b, H4-RET, HLA-DR, HM1.24, human chorionic gonadotropin (HCG) HER2, HER3, HMGB-1, HIF-1, HSP70-2M, HST-2, HTgp-175, la, IGF-1R, IFN-g, IFN-α, IFN-b, IFN-1, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KLK2, KSA, KS-1-antigen, KS1-4, LAGE-1a, Le-Y, LDR/FUT, M344, MA-50, macrophage migration inhibitory factor (MIF), MAGE, MAGE-1, MAGE-3, MAGE-4, MAGE-5, MAGE-6, MART-1, MART-2, TRAG-3, MCP-1, MIP-1A, MIP-1B, MIF, MG7-Ag, MOV18, MUC1, MUC2, MUC3, MUC4, MUC5ac, MUC13, MUC16, MUM-1/2, MUM-3, MYL-RAR, NB/70K, Nm23H1, NuMA, NCA66, NCA95, NCA90, NY-ESO-1, p15, p16, p185erbB2, p180erbB3, PAM4 antigen, pancreatic cancer mucin, PD-1, PD-L1, PD-L2, PI5, placental growth factor, p53, PLAGL2, Pmel17 prostatic acid phosphatase, PSA, PRAME, PSMA, PlGF, ILGF, ILGF-1R, IL-6, IL-25, RCAS1, RS5, RAGE, RANTES, Ras, T101, SAGE, S100, SLAMF7, survivin, survivin-2B, SDDCAG16, TA-90\Mac2 binding protein, TAAL6, TAC, TAG-72, TLP, tenascin, TMEFF2, TRAIL receptors, TRP-1, TRP-2, TSP-180, VEGFR, ED-B fibronectin, WT-1, 17-1A-antigen, C3, C3a, C3b, C5a, C5, bcl-2, K-ras, tumor neoantigen, a viral antigen associated with cancer, FcγRIIB, IL-12β2R, CD28, CD56, CD11c, CD66b, CD41, CD61, CD62, CD235a, CD146, CD326, or CD203c.

39. A kit, comprising the isolated molecule of claim 1.

40. The kit of claim 39, further comprising means for diluting or administering the isolated molecule of claim 1.

41. A pharmaceutical composition, comprising the isolated molecule of claim 1 and a pharmaceutically acceptable excipient.

42. A method of selectively activating or recruiting CD8⁺ CTLs towards an undesired cell, comprising: contacting a population of lymphocytes with an isolated molecule of claim 1, wherein optionally the selective activation or recruitment of CD8⁺ CTLs comprises in vitro selective activation or recruitment of CD8⁺ CTLs; wherein optionally the selective activation or recruitment of CD8⁺ CTLs comprises ex vivo selective activation or recruitment of CD8⁺ CTLs; wherein optionally the selective activation or recruitment of CD8⁺ CTLs comprises in vivo selective activation or recruitment of CD8⁺ CTLs.

43.-45. (canceled)

46. A method of selectively activating or recruiting CD8⁺ CTLs towards an undesired cell in a subject, or providing an improved T cell redirection therapy for a subject in need thereof, or targeting CD8+ CTLs to an undesired cell in a subject, or treating a cancer in a subject, or enhancing a CD8⁺ CTL response against an undesired cell in a subject, comprising administering to the subject an isolated molecule of claim 1.

47.-50. (canceled)

51. The method of claim 46, wherein the subject has a cancer, an infection, or an immune-mediated disease,
wherein optionally the cancer is a hematological malignancy or a solid tumor, wherein optionally the hematological malignancy comprises acute lymphoblastic leukemia, acute myeloid leukemia, anaplastic large-cell lymphoma, Burkitt's lymphoma, chronic lymphocytic leukemia, chronic myeloid leukemia, diffuse large B-cell lymphoma, dendritic cell neoplasm, follicular lymphoma, hairy cell leukemia, Hodgkin's lymphoma, leukemia, B cell leukemia, T cell leukemia, light chain amyloidosis, lymphoma, B cell lymphoma, NK cell lymphoma, T cell lymphoma, mantle-cell lymphoma, marginal zone B-cell lymphoma, monoclonal gammopathy of undetermined significance, mucosa-associated lymphatic tissue lymphoma, multiple myeloma, myelodysplastic syndrome, non-Hodgkin's lymphoma, plasma cell leukemia, precursor B-cell lymphoblastic leukemia, smoldering multiple myeloma, Waldenstrom's macroglobulinemia, B cell malignancy, T cell malignancy, NK cell malignancy, or any combination thereof;
wherein optionally the cancer is a solid tumor cancer, wherein optionally the solid tumor cancer comprises adenocarcinoma, anal cancer, basal cell carcinoma, biliary tract cancer, bladder cancer, bone cancer, breast cancer, cancer associated with infection, cancer of the adrenal gland, cancer of the endocrine system, cancer of the head or neck, cancer of the parathyroid gland, cancer of the penis, cancer of the thyroid gland, cancer of the urethra, cervical cancer, carcinoma of the breast, carcinoma of the fallopian tubes, carcinoma of the liver, carcinoma of the lung, carcinoma of the prostate, carcinoma of the renal pelvis, carcinoma of the vagina, carcinoma of the vulva, choriocarcinoma, clear cell carcinoma, colon cancer, colon carcinoma, colorectal cancer, connective tissue cancer, cutaneous or intraocular malignant melanoma, environmentally induced cancer, gastric cancer, gastrointestinal cancer, glioma, glioblastoma, endometrial cancer, epithelial cancer, esophageal cancer, eye cancer, larynx cancer, liver cancer, hepatocellular carcinoma, hormone refractory prostate adenocarcinoma, Kaposi's sarcoma, kidney cancer, lung cancer gastro-esophageal cancer, melanoma, mesothelioma, Merkel cell cancer, neuroblastoma, non-small cell lung cancer (NSCLC), osteosarcoma, ovarian cancer, pancreatic cancer, prostate cancer, rectal cancer, renal cell carcinoma, retinoblastoma rhabdomyosarcoma, squamous cell cancer, soft tissue sarcoma, solid tumors of childhood, spinal axis tumor, stomach cancer, testicular cancer, thyroid cancer, uterine cancer, urothelial carcinoma or sarcomas, or any combination thereof;
wherein optionally the infection comprises infection with adenovirus, arboviral encephalitis virus, coronavirus, coxsackie virus, cytomegalovirus (CMV), dengue virus, echovirus, Epstein Barr virus, flaviviruses, human immunodeficiency virus (HIV), hepatitis A virus, hepatitis B virus, hepatitis C virus, herpes virus, HTLV virus, influenza virus, JC virus, measles virus, molluscum virus, mumps virus, papillomavirus, parvovirus, poliovirus, rabies virus, respiratory syncytial virus, rhinovirus, rotavirus, rubella virus or vaccinia virus, bacteria, virus, fungi, protozoa, parasite or prion, or any combination thereof; and
wherein optionally the immune-mediated disease comprises systemic lupus erythematosus (SLE), ankylosing spondylitis, Chagas disease, chronic obstructive pulmonary disease, Crohn's Disease, dermatomyositis, diabetes mellitus type 1, endometriosis, Goodpasture's syndrome, Graves' disease, Guillain-Barre syndrome (GBS), Hashimoto's disease, hidradenitis suppurativa, Kawasaki disease, IgA nephropathy, idiopathic thrombocytopenic purpura, interstitial cystitis, mixed connective tissue disease, morphea, multiple sclerosis, myasthenia gravis, narcolepsy, neuromyotonia, pemphigus vulgaris, pernicious anaemia, psoriasis, psoriatic arthritis, polymyositis, primary biliary cirrhosis, relapsing polychondritis, rheumatoid arthritis (RA), sarcoidosis, schizophrenia, scleroderma, Sjogren's syndrome, temporal arteritis, ulcerative colitis, vasculitis, vitiligo, Wegener's granulomatosis, IgG4-related disease, anti-synthetase syndrome, and autoimmunity associated with immunodeficiency including chronic variable immunodeficiency, Wiskott-Aldrich syndrome, Good syndrome, IgA deficiency, Hyper IgM syndrome, complement disorders, seropositive RA, SLE, postmyocardial infarction syndrome, subacute bacterial endocarditis, anti-glomerular basement membrane nephritis, autoimmune hepatitis, primary biliary cirrhosis, alopecia areata, bullous pemphigoid, cicatricial pemphigoid, dermatitis herpetiformis, gestational pemphigoid, pemphigus vulgaris, systemic scleroderma, Addison's disease, autoimmune polyendocrine syndrome type 2, autoimmune pancreatitis, diabetes mellitus type 1, autoimmune thyroiditis, Graves' disease, Sjogren's syndrome, celiac disease, antiphospholipid syndrome, autoimmune thrombocytopenic purpura, cold agglutinin disease, pernicious anemia, thrombocytopenia, adult onset Still's disease, CREST syndrome, drug-induced lupus, enthesitis-related arthritis, juvenile arthritis, mixed connective tissue disease, palindromic rheumatism, Parry Romberg syndrome, rheumatic fever, undifferentiated connective tissue disease, dermatomysitis, myasthenia gravis, neuromyotonia, paraneoplastic cerebellar degeneration, polymyositis, Bickerstaff s encephalitis, chronic inflammatory demyelinating polyneuropathy, Guillain-Barre syndrome, Hashimoto's encephalopathy, Lambert-Eaton myasthenic syndrome, multiple sclerosis, progressive inflammatory neuropathy, Stiff person syndrome, autoimmune uveitis, neuromyelitis optica, symphathetic ophthalmia, Meniere's disease, anti-neutrophil cytoplasmic antibody-associated vasculitis, Churg-Strauss syndrome, Henoch-Schonlein purpura, microscopic polyangiitis, urticarial vasculitis, and vasculitis. Examples of autoantibody-associated autoimmune conditions include gastritis and POEMS syndrome. Examples of autoantibody-associated (non-autoimmune) diseases include agammaglobulinemia, amyotrophic lateral sclerosis, Castleman's disease, cutaneous leukocytoclastic angiitis, eczema, eosinophilic gastroenteritis, erythroblastosis fetalis, fibrodysplasia ossificans progressive, hypogammaglobulinemia, idiopathic pulmonary fibrosis, IgA nephropathy, Majeed syndrome, narcolepsy, Rasmussen's encephalitis, spondyloarthropathy or Sweet's syndrome, or any combination thereof.

52.-56. (canceled)

57. A system comprising a means for selective activation or recruitment of CD8⁺ CTLs.

58. A composition comprising an antibody comprising a first antigen binding domain and a second antigen binding domain, and means of claim 57.

59. A composition for enhancing an immune response against an antigen expressed by an undesired cell, comprising means of claim 57.

60. A composition for treating a cancer in subject, comprising means of claim 57.

61. A system comprising a means for providing an improved T cell redirecting therapeutic treatment to a subject.

62. The system of claim 61, wherein the T cell redirecting therapeutic treatment comprises administration of an isolated molecule of claim 1.

63. The system of claim 61, wherein the T cell redirecting therapeutic comprising a means for improving safety of the T cell redirecting therapeutic.

64. A process for generating an improved T cell redirecting therapeutic, comprising:
a) a step for performing a function of designing the T cell redirecting therapeutic comprising the means of claim 61; and
b) a step for performing a function of producing the T cell redirecting therapeutic comprising the means of claim 61.

65. A method of isolating, separating, purifying, sorting, selecting or capturing a CD8+ CTL comprising:
a) providing a sample comprising the CD8+ CTL;
b) contacting the sample with an isolated molecule of claim 1; and
c) isolating, separating, purifying, sorting, selecting or capturing the CD8⁺ CTL bound to the isolated molecule;
wherein optionally, the sample is a blood sample or a tissue sample.

66. (canceled)

67. The method of claim 65, wherein the method is conducted in suspension or on a solid support.

68. The method of claim 65, wherein the method is conducted using particles, microfluidics, fluorescent cell sorting, chips, columns or surfaces.
